SEQUENCE LISTING (1466 sequences)

SEQ ID NO: 1 - DNA, 27 bp, synthetic construct
  misc_feature 1..27: note = Description of Artificial Sequence: Synthetic oligonucleotide
  source 1..27: mol_type = other DNA
gatcttattctactcctgtgaaaggat

SEQ ID NO: 2 - DNA, 27 bp, synthetic construct
  misc_feature 1..27: note = Description of Artificial Sequence: Synthetic oligonucleotide
  source 1..27: mol_type = other DNA
aacgttgttctgagtagctttcaggat

SEQ ID NO: 3 - DNA, 27 bp, synthetic construct
  misc_feature 1..27: note = Description of Artificial Sequence: Synthetic oligonucleotide
  source 1..27: mol_type = other DNA
gatcattcttgtggcagtaaggaggat

SEQ ID NO: 4 - DNA, 27 bp, synthetic construct
  misc_feature 1..27: note = Description of Artificial Sequence: Synthetic oligonucleotide
  source 1..27: mol_type = other DNA
tcaaaagctaccggttacctgaagggt

SEQ ID NO: 5 - DNA, 23 bp, synthetic construct
  misc_feature 1..23: note = Description of Artificial Sequence: Synthetic oligonucleotide
  source 1..23: mol_type = other DNA
gagatattcacaattacaactgg

SEQ ID NO: 6 - DNA, 23 bp, synthetic construct
  misc_feature 1..23: note = Description of Artificial Sequence: Synthetic oligonucleotide
  source 1..23: mol_type = other DNA
gagatactcacaattacaactgg

SEQ ID NO: 7 - DNA, 27 bp, Homo sapiens
  source 1..27: mol_type = other DNA
agaaatagatgtagattgaggtagaat

SEQ ID NO: 8 - DNA, 27 bp, Homo sapiens
  source 1..27: mol_type = other DNA
ataaggaaatacaaaaactggatgggt

SEQ ID NO: 9 - DNA, 27 bp, Homo sapiens
  source 1..27: mol_type = other DNA
gataataaggaaatacaaaaactggat

SEQ ID NO: 10 - DNA, 27 bp, Homo sapiens
  source 1..27: mol_type = other DNA
tcagatttcatgtgtgaagaatggaat

SEQ ID NO: 11 - DNA, 27 bp, Homo sapiens
  source 1..27: mol_type = other DNA
gaaaatcagatttcatgtgtgaagaat

SEQ ID NO: 12 - DNA, 27 bp, Homo sapiens
  source 1..27: mol_type = other DNA
cttttgacagtttttaaggcggggagt

SEQ ID NO: 13 - DNA, 27 bp, Homo sapiens
  source 1..27: mol_type = other DNA
tcaaaagctaccggttacctgaagggt

SEQ ID NO: 14 - DNA, 27 bp, Homo sapiens
  source 1..27: mol_type = other DNA
ttttaaggcggggagtcacatgggagt

SEQ ID NO: 15 - DNA, 27 bp, Homo sapiens
  source 1..27: mol_type = other DNA
cggggagtcacatgggagtcacagggt

SEQ ID NO: 16 - DNA, 27 bp, Homo sapiens
  source 1..27: mol_type = other DNA
agtcacatgggagtcacagggtaggat

SEQ ID NO: 17 - DNA, 27 bp, Homo sapiens
  source 1..27: mol_type = other DNA
tcacagggtaggattcatgtttagaat

SEQ ID NO: 18 - DNA, 27 bp, Homo sapiens
  source 1..27: mol_type = other DNA
gatcattcttgtggcagtaaggaggat

SEQ ID NO: 19 - DNA, 27 bp, Homo sapiens
  source 1..27: mol_type = other DNA
acaagaatgatcattctaaacatgaat

SEQ ID NO: 20 - DNA, 27 bp, Homo sapiens
  source 1..27: mol_type = other DNA
tgtaagactggagatagagacaggaat

SEQ ID NO: 21 - DNA, 27 bp, Homo sapiens
  source 1..27: mol_type = other DNA
ttacatcctccttactgccacaagaat

SEQ ID NO: 22 - DNA, 27 bp, Homo sapiens
  source 1..27: mol_type = other DNA
taaaactaagacactgccaatagggat

SEQ ID NO: 23 - DNA, 27 bp, Homo sapiens
  source 1..27: mol_type = other DNA
tttaacgttatcattttcccaaagagt

SEQ ID NO: 24 - DNA, 27 bp, Homo sapiens
  source 1..27: mol_type = other DNA
catcttgtggataatgtatcaatgagt

SEQ ID NO: 25 - DNA, 27 bp, Homo sapiens
  source 1..27: mol_type = other DNA
ctaggactttctaatgctggagaggat

SEQ ID NO: 26 - DNA, 27 bp, Homo sapiens
  source 1..27: mol_type = other DNA
gtcctaggcaagagacatcttgtggat

SEQ ID NO: 27 - DNA, 27 bp, Homo sapiens
  source 1..27: mol_type = other DNA
gaggataggacagaggacatggagaat

SEQ ID NO: 28 - DNA, 27 bp, Homo sapiens
  source 1..27: mol_type = other DNA
aaacatgactcataatttagtaggaat

SEQ ID NO: 29 - DNA, 27 bp, Homo sapiens
  source 1..27: mol_type = other DNA
tttcaggattcctactaaattatgagt

SEQ ID NO: 30 - DNA, 27 bp, Homo sapiens
  source 1..27: mol_type = other DNA
tcagaacaacgttttcatttactgaat

SEQ ID NO: 31 - DNA, 27 bp, Homo sapiens
  source 1..27: mol_type = other DNA
aacgttgttctgagtagctttcaggat

SEQ ID NO: 32 - DNA, 27 bp, Homo sapiens
  source 1..27: mol_type = other DNA
tcagtaaatgaaaacgttgttctgagt

SEQ ID NO: 33 - DNA, 27 bp, Homo sapiens
  source 1..27: mol_type = other DNA
cagcaaaagcttttgagctaatagaat

SEQ ID NO: 34 - DNA, 27 bp, Homo sapiens
  source 1..27: mol_type = other DNA
gatcttattctactcctgtgaaaggat

SEQ ID NO: 35 - DNA, 27 bp, Homo sapiens
  source 1..27: mol_type = other DNA
tcctgtgaaaggatcttagataagaat

SEQ ID NO: 36 - DNA, 27 bp, Homo sapiens
  source 1..27: mol_type = other DNA
tctaagatcctttcacaggagtagaat

SEQ ID NO: 37 - DNA, 27 bp, Homo sapiens
  source 1..27: mol_type = other DNA
tcttatctaagatcctttcacaggagt

SEQ ID NO: 38 - DNA, 27 bp, Homo sapiens
  source 1..27: mol_type = other DNA
agaaatagaggcttatggattttggat

SEQ ID NO: 39 - DNA, 27 bp, Homo sapiens
  source 1..27: mol_type = other DNA
cctcatcagaaatagaggcttatggat

SEQ ID NO: 40 - DNA, 27 bp, Homo sapiens
  source 1..27: mol_type = other DNA
ttctgtcctcagtaaaaggtatagagt

SEQ ID NO: 41 - DNA, 23 bp, Homo sapiens
  source 1..23: mol_type = other DNA
attgaggtagaatcaagaagagg

SEQ ID NO: 42 - DNA, 23 bp, Homo sapiens
  source 1..23: mol_type = other DNA
aaagaaatagatgtagattgagg

SEQ ID NO: 43 - DNA, 23 bp, Homo sapiens
  source 1..23: mol_type = other DNA
aggaaatacaaaaactggatggg

SEQ ID NO: 44 - DNA, 23 bp, Homo sapiens
  source 1..23: mol_type = other DNA
aaggaaatacaaaaactggatgg

SEQ ID NO: 45 - DNA, 23 bp, Homo sapiens
  source 1..23: mol_type = other DNA
taataaggaaatacaaaaactgg

SEQ ID NO: 46 - DNA, 23 bp, Homo sapiens
  source 1..23: mol_type = other DNA
gaagaatggaatagataataagg

SEQ ID NO: 47 - DNA, 23 bp, Homo sapiens
  source 1..23: mol_type = other DNA
cagatttcatgtgtgaagaatgg

SEQ ID NO: 48 - DNA, 23 bp, Homo sapiens
  source 1..23: mol_type = other DNA
gtaaaggttcatgagactagagg

SEQ ID NO: 49 - DNA, 23 bp, Homo sapiens
  source 1..23: mol_type = other DNA
tgttgctttttgagaggtaaagg

SEQ ID NO: 50 - DNA, 23 bp, Homo sapiens
  source 1..23: mol_type = other DNA
aattattgttgctttttgagagg

SEQ ID NO: 51 - DNA, 23 bp, Homo sapiens
  source 1..23: mol_type = other DNA
atttgacaccacatgcactgtgg

SEQ ID NO: 52 - DNA, 23 bp, Homo sapiens
  source 1..23: mol_type = other DNA
tggtgtcaaatatggtgcttagg

SEQ ID NO: 53 - DNA, 23 bp, Homo sapiens
  source 1..23: mol_type = other DNA
agtgcatgtggtgtcaaatatgg

SEQ ID NO: 54 - DNA, 23 bp, Homo sapiens
  source 1..23: mol_type = other DNA
gtggagagccacagtgcatgtgg

SEQ ID NO: 55 - DNA, 23 bp, Homo sapiens
  source 1..23: mol_type = other DNA
gtagcttttgacagtttttaagg

SEQ ID NO: 56 - DNA, 23 bp, Homo sapiens
  source 1..23: mol_type = other DNA
gcttttgacagtttttaaggcgg

SEQ ID NO: 57 - DNA, 23 bp, Homo sapiens
  source 1..23: mol_type = other DNA
cttttgacagtttttaaggcggg

SEQ ID NO: 58 - DNA, 23 bp, Homo sapiens
  source 1..23: mol_type = other DNA
ttttgacagtttttaaggcgggg

SEQ ID NO: 59 - DNA, 23 bp, Homo sapiens
  source 1..23: mol_type = other DNA
aaagctaccggttacctgaaggg

SEQ ID NO: 60 - DNA, 23 bp, Homo sapiens
  source 1..23: mol_type = other DNA
aaaagctaccggttacctgaagg

SEQ ID NO: 61 - DNA, 23 bp, Homo sapiens
  source 1..23: mol_type = other DNA
ttttaaggcggggagtcacatgg

SEQ ID NO: 62 - DNA, 23 bp, Homo sapiens
  source 1..23: mol_type = other DNA
tttaaggcggggagtcacatggg

SEQ ID NO: 63 - DNA, 23 bp, Homo sapiens
  source 1..23: mol_type = other DNA
taaaaactgtcaaaagctaccgg

SEQ ID NO: 64 - DNA, 23 bp, Homo sapiens
  source 1..23: mol_type = other DNA
gggagtcacatgggagtcacagg

SEQ ID NO: 65 - DNA, 23 bp, Homo sapiens
  source 1..23: mol_type = other DNA
ggagtcacatgggagtcacaggg

SEQ ID NO: 66 - DNA, 23 bp, Homo sapiens
  source 1..23: mol_type = other DNA
tcacatgggagtcacagggtagg

SEQ ID NO: 67 - DNA, 23 bp, Homo sapiens
  source 1..23: mol_type = other DNA
gtttagaatgatcattcttgtgg

SEQ ID NO: 68 - DNA, 23 bp, Homo sapiens
  source 1..23: mol_type = other DNA
tgatcattcttgtggcagtaagg

SEQ ID NO: 69 - DNA, 23 bp, Homo sapiens
  source 1..23: mol_type = other DNA
tcattcttgtggcagtaaggagg

SEQ ID NO: 70 - DNA, 23 bp, Homo sapiens
  source 1..23: mol_type = other DNA
cagtaaggaggatgtaagactgg

SEQ ID NO: 71 - DNA, 23 bp, Homo sapiens
  source 1..23: mol_type = other DNA
gtaagactggagatagagacagg

SEQ ID NO: 72 - DNA, 23 bp, Homo sapiens
  source 1..23: mol_type = other DNA
ggagatagagacaggaataatgg

SEQ ID NO: 73 - DNA, 23 bp, Homo sapiens
  source 1..23: mol_type = other DNA
attttttattatctttattgtgg

SEQ ID NO: 74 - DNA, 23 bp, Homo sapiens
  source 1..23: mol_type = other DNA
aaaactaagacactgccaatagg

SEQ ID NO: 75 - DNA, 23 bp, Homo sapiens
  source 1..23: mol_type = other DNA
aaactaagacactgccaataggg

SEQ ID NO: 76 - DNA, 23 bp, Homo sapiens
  source 1..23: mol_type = other DNA
aagacactgccaatagggatagg

SEQ ID NO: 77 - DNA, 23 bp, Homo sapiens
  source 1..23: mol_type = other DNA
gagatattcacaattacaactgg

SEQ ID NO: 78 - DNA, 23 bp, Homo sapiens
  source 1..23: mol_type = other DNA
tatctcatacctatccctattgg

SEQ ID NO: 79 - DNA, 23 bp, Homo sapiens
  source 1..23: mol_type = other DNA
gaaagatgaaaaatactctttgg

SEQ ID NO: 80 - DNA, 23 bp, Homo sapiens
  source 1..23: mol_type = other DNA
aaagatgaaaaatactctttggg

SEQ ID NO: 81 - DNA, 23 bp, Homo sapiens
  source 1..23: mol_type = other DNA
ccacaagatgtctcttgcctagg

SEQ ID NO: 82 - DNA, 23 bp, Homo sapiens
  source 1..23: mol_type = other DNA
tgcctaggactttctaatgctgg

SEQ ID NO: 83 - DNA, 23 bp, Homo sapiens
  source 1..23: mol_type = other DNA
aggactttctaatgctggagagg

SEQ ID NO: 84 - DNA, 23 bp, Homo sapiens
  source 1..23: mol_type = other DNA
cctaggcaagagacatcttgtgg

SEQ ID NO: 85 - DNA, 23 bp, Homo sapiens
  source 1..23: mol_type = other DNA
tttctaatgctggagaggatagg

SEQ ID NO: 86 - DNA, 23 bp, Homo sapiens
  source 1..23: mol_type = other DNA
tgctggagaggataggacagagg

SEQ ID NO: 87 - DNA, 23 bp, Homo sapiens
  source 1..23: mol_type = other DNA
agaggataggacagaggacatgg

SEQ ID NO: 88 - DNA, 23 bp, Homo sapiens
  source 1..23: mol_type = other DNA
ctccagcattagaaagtcctagg

SEQ ID NO: 89 - DNA, 23 bp, Homo sapiens
  source 1..23: mol_type = other DNA
aggacagaggacatggagaatgg

SEQ ID NO: 90 - DNA, 23 bp, Homo sapiens
  source 1..23: mol_type = other DNA
aaattatcaccacactaaatagg

SEQ ID NO: 91 - DNA, 23 bp, Homo sapiens
  source 1..23: mol_type = other DNA
aaaattatgcctatttagtgtgg

SEQ ID NO: 92 - DNA, 23 bp, Homo sapiens
  source 1..23: mol_type = other DNA
gtaacataatcacctctctttgg

SEQ ID NO: 93 - DNA, 23 bp, Homo sapiens
  source 1..23: mol_type = other DNA
ctgctgcttttgccaaagagagg

SEQ ID NO: 94 - DNA, 23 bp, Homo sapiens
  source 1..23: mol_type = other DNA
aacatgactcataatttagtagg

SEQ ID NO: 95 - DNA, 23 bp, Homo sapiens
  source 1..23: mol_type = other DNA
cgttgttctgagtagctttcagg

SEQ ID NO: 96 - DNA, 23 bp, Homo sapiens
  source 1..23: mol_type = other DNA
attagctcaaaagcttttgctgg

SEQ ID NO: 97 - DNA, 23 bp, Homo sapiens
  source 1..23: mol_type = other DNA
tcttattctactcctgtgaaagg

SEQ ID NO: 98 - DNA, 23 bp, Homo sapiens
  source 1..23: mol_type = other DNA
cttatctaagatcctttcacagg

SEQ ID NO: 99 - DNA, 23 bp, Homo sapiens
  source 1..23: mol_type = other DNA
ataagcctctatttctgatgagg

SEQ ID NO: 100 - DNA, 23 bp, Homo sapiens
  source 1..23: mol_type = other DNA
aaatagaggcttatggattttgg

SEQ ID NO: 101 - DNA, 23 bp, Homo sapiens
  source 1..23: mol_type = other DNA
atgaggaagatgaacaaatcagg

SEQ ID NO: 102 - DNA, 23 bp, Homo sapiens
  source 1..23: mol_type = other DNA
tcatcagaaatagaggcttatgg

SEQ ID NO: 103 - DNA, 23 bp, Homo sapiens
  source 1..23: mol_type = other DNA
atcttcctcatcagaaatagagg

SEQ ID NO: 104 - DNA, 23 bp, Homo sapiens
  source 1..23: mol_type = other DNA
cttgttctgtcctcagtaaaagg

SEQ ID NO: 105 - DNA, 23 bp, Homo sapiens
  source 1..23: mol_type = other DNA
taaaaggtatagagttcaagtgg

SEQ ID NO: 106 - DNA, 23 bp, Homo sapiens
  source 1..23: mol_type = other DNA
gaactctataccttttactgagg

SEQ ID NO: 107 - DNA, 23 bp, Homo sapiens
  source 1..23: mol_type = other DNA
tggctgtaagataactacaaagg

SEQ ID NO: 108 - DNA, 23 bp, Homo sapiens
  source 1..23: mol_type = other DNA
ggctgtaagataactacaaaggg

SEQ ID NO: 109 - DNA, 23 bp, Homo sapiens
  source 1..23: mol_type = other DNA
taagataactacaaagggtctgg

SEQ ID NO: 110 - DNA, 4271 bp, synthetic construct
  misc_feature 1..4271: note = Description of Artificial Sequence: Synthetic polynucleotide
  source 1..4271: mol_type = other DNA
ccatgggcgcatgcaaattacgcgctgtgctttgtgggaaatcaccctaaacgaaaaatttattcctctttcgagccttatagtggcggccggtctacatcctgaagatcattcttgtggcagtaaggttttagtactctggaaacagaatctactaaaacaaggcaaaatgccgtgtttatctcgtcaacttgttggcgagattttttgttaacgcgcatgcaaattacgcgctgtgctttgtgggaaatcaccctaaacgaaaaatttattcctctttcgagccttatagtggcggccggtctacatcctgaatcaaaagctaccggttacctggttttagtactctggaaacagaatctactaaaacaaggcaaaatgccgtgtttatctcgtcaacttgttggcgagatttttttctagacacacaaaaaaccaacacacagatgtaatgaaaataaagatattttattaggctgatcagcgagctctaggaattcttaggatccctttttcttttttgcctggccggcctttttcgtggccgccggccttttgccctttttgatgatctgagggtgcttcttagatttcacttcatacaggttgcccagaatgtctgtgctgtacttcttaatgctctgggtcttggaggcgattgtcttaatgatcctggggggcctcttgtcgttcatgttttccaggtactcgcggtaggtgatgtcgatcatgttcacttcgatccggttcagcaggtcgttgttcacgccgatcactctatacagctcgccgttgatcttgatcagatcgttgttgtagaaggaggcgataaactcggcctggttgctgatcttcttcagcttcttagcttcctcatagcacttgctattcacttcgtagtagttttcttttttgatcacatccagattcttcacggtcacgaacttgtacacgccattgtccaggtacacgtcgaatctgtagggcttcagggacagcttcacgaccttgtttctgctgttggggtagtcgtcggtgatgtccagatgggcgttcagtttgttgccgtaatacttaatcttcttgatcacggggccgttgtcctttttggagtacttggtcaggtagttcccggtttcctcgtagtacttgtacaggggattcttctcgtcgccgtactgttccataatcagcttcagtttctggtaggtctgggggtcgtggtggtacatcagcagcttttcggggctcttgttgatcagctttttcagcttgtcattgtccttgtcgtacaggccgttcagattgttcacgatcagggtgttgcccttgtcgtccttccgggtggagtacagggtgtcgttaatcagctctctattaggcttcttgtccacccggtggctgtacttgtagtccttgaagtccttaatgtgcttgatctggtggggggtgatgaagatctctttgtactcctgctcggtttcgatctcgggcatgctctcggcctgcttttcctcgaacatctggttttccatcacttttttggccttgtccagtttcttccactctttgaagatgaaatcggcgttggcaatgatcagggcgtcctcggcgtggtgcttgtaccccttgttccgctctttcttaaacttccacttccgccgcagaaagctggtgaagccgccattgatggacttcactttcacgtccaggttgttcactctgaagtagctccgcagcaggttcatcaggcctctggtggcgtatctggtatccaccaggttccggttgatgaagtctttctgcacggagaacctgttgatgtcccgttcttccagcagatactctttcttggtcttgctgattctgcccttgcccttggccagattcaggatgtgcttcttgaaggtttcgtagctgatcttgctgtcgctgctgctcaggtactggaatggggtccggttgcccttcttgctgttttcttcctgcttcacgagcaccttgttgttgaagctgttgtcgaaggacacgcttctggggatgatgtggtccacctcatagttgaaggggttgttcagcagatcttccagagggatggcttccaggctgtacaggcacttgccttcctgcatgtcgtgcagcttgatcttctcgatcaggtacttggcgttctctttgccggtggtccggatgatttcctcgatccgctcgttggtctgccggttccgcttctgcatctcgttgatcattttctgggcgtccttggagttcttctcgcgggccagctcgataatgatgtcgttgggcaggccgtacttcttgatgatggcgttgatcactttgatgctctggatgaagcttctcttcacgacggggctcaggatgaagtcgtccaccagggtggtggggatctctttctgctgggacaggtccaccttcttgggcaccagcttcagccggttgaagatagcgatctggttgtcgttggtgtgccacagctcgtccaggatcaggttgatggccttcaggctcaggttgtgggtgccggtatagcccttcagattagagatctgctcgatctcttcctgggtcagctcggagttcagattggtcagttcttcctggatgtcctcgctgctctggtagatggtcaggatcttggcaatctgatccagcagctcggcgttctcaataatctctttccgggcggtaatgtccttgatgtcgtggtacaccttcaggttggtgaactcgggcttgccggtgctggtcactctgtagcccttaatatcctcttcgttcacgaggatttctttggcgatctgcttcagggtgggcttcttcttctgcttgaacacgttctcgatgatctggaacttctcgtaatattccagcttctcgttctcgtccctggtgatcacgagattgttcaggtcgttcagggcgttgtacaggtcggcgttgtaggcgtacttcacgctccgcagttcctcggggaagtaggtgcagtggcccatcagcatctcgtaccattctttgatgtccttccagccgaaggggctgccctcgccaggtccctcatagtaggtccgccgggtttccagcaggtcgatgtaggtgtcgatgaagctctggtccagctggtggtaggccttctgcaccttcagcagctgtttggcttctttcacgtagtcgctggtcttgaatctgttgatgctgccccgcacttcgccgtctttcttcagccgttccagctgcagttcggccacgtatttctcttccagggccttgctgttccggctgatctgctctttggtggacagctcgttgccggtgtcctcttccacctcgttcacgttgtgcacgcctcttctcttggccaggtgcagcagggcggcagagaactcttcctcgctcagcttctggctcaggcccttcactctggcctcgtaggggttgatgccgctcagctcgctgtggtcggtcagcaggttgtagtcgaacagcagcttcttcactctctggattctatgccgcctccgccgcttcagccttctggcgcctctcttgctccgcctgccctcgttgttttccacgttggcctctttgaacagccgcacgccggcatcgatcacgtcccgtgtctcgtagtcgatgatgccgtagcccacgctggtgatgccgatgtccaggcccaggatgtagttccgcttggctgctgggactccgtggataccgaccttccgcttcttctttggggccatgtggcggctcttgaaggacgacgtcatcatcccttgcccggatgcgcgggcttcttgtctagcacaggagcctggggtagagcgcatgcaaattacgcgctgtgctttgtgggaaatcaccctaaacgaaaaatttattcctctttcgagccttatagtggcggccggtctacatcctgaagatcttattctactcctgtgagttttagtactctggaaacagaatctactaaaacaaggcaaaatgccgtgtttatctcgtcaacttgttggcgagattttttgatatcgcgcatgcaaattacgcgctgtgctttgtgggaaatcaccctaaacgaaaaatttattcctctttcgagccttatagtggcggccggtctacatcctgaaaacgttgttctgagtagctttgttttagtactctggaaacagaatctactaaaacaaggcaaaatgccgtgtttatctcgtcaacttgttggcgagattttttctcgaggcggccgc

SEQ ID NO: 111 - DNA, 23 bp, Homo sapiens
  source 1..23: mol_type = other DNA
cctggccatcgagcggtacgtgg

SEQ ID NO: 112 - DNA, 23 bp, Homo sapiens
  source 1..23: mol_type = other DNA
ggccatcgagcggtacgtggtgg

SEQ ID NO: 113 - DNA, 23 bp, Homo sapiens
  source 1..23: mol_type = other DNA
ccacgtaccgctcgatggccagg

SEQ ID NO: 114 - DNA, 23 bp, Homo sapiens
  source 1..23: mol_type = other DNA
caccaccacgtaccgctcgatgg

SEQ ID NO: 115 - DNA, 23 bp, Homo sapiens
  source 1..23: mol_type = other DNA
cctggccatcgaggggtacgtgg

SEQ ID NO: 116 - DNA, 23 bp, Homo sapiens
  source 1..23: mol_type = other DNA
ggccatcgaggggtacgtggtgg

SEQ ID NO: 117 - DNA, 23 bp, Homo sapiens
  source 1..23: mol_type = other DNA
ccacgtacccctcgatggccagg

SEQ ID NO: 118 - DNA, 23 bp, Homo sapiens
  source 1..23: mol_type = other DNA
caccaccacgtacccctcgatgg

SEQ ID NO: 119 - DNA, 23 bp, Homo sapiens
  source 1..23: mol_type = other DNA
cctggccatcgagtggtacgtgg

SEQ ID NO: 120 - DNA, 23 bp, Homo sapiens
  source 1..23: mol_type = other DNA
ggccatcgagtggtacgtggtgg

SEQ ID NO: 121 - DNA, 23 bp, Homo sapiens
  source 1..23: mol_type = other DNA
ccacgtaccactcgatggccagg

SEQ ID NO: 122 - DNA, 23 bp, Homo sapiens
  source 1..23: mol_type = other DNA
caccaccacgtaccactcgatgg

SEQ ID NO: 123 - DNA, 23 bp, Homo sapiens
  source 1..23: mol_type = other DNA
cctggccatcgagctgtacgtgg

SEQ ID NO: 124 - DNA, 23 bp, Homo sapiens
  source 1..23: mol_type = other DNA
ggccatcgagctgtacgtggtgg

SEQ ID NO: 125 - DNA, 23 bp, Homo sapiens
  source 1..23: mol_type = other DNA
ccacgtacagctcgatggccagg

SEQ ID NO: 126 - DNA, 23 bp, Homo sapiens
  source 1..23: mol_type = other DNA
caccaccacgtacagctcgatgg

SEQ ID NO: 127 - DNA, 100 bp, synthetic construct
  misc_feature 1..100: note = Description of Artificial Sequence: Synthetic polynucleotide
  source 1..100: mol_type = other DNA
cctggccatcgagcggtacggttttagagctagaaatagcaagttaaaataaggctagtccgttatcaacttgaaaaagtggcaccgagtcggtgctttt

SEQ ID NO: 128 - DNA, 100 bp, synthetic construct
  misc_feature 1..100: note = Description of Artificial Sequence: Synthetic polynucleotide
  source 1..100: mol_type = other DNA
ggccatcgagcggtacgtgggttttagagctagaaatagcaagttaaaataaggctagtccgttatcaacttgaaaaagtggcaccgagtcggtgctttt

SEQ ID NO: 129 - DNA, 100 bp, synthetic construct
  misc_feature 1..100: note = Description of Artificial Sequence: Synthetic polynucleotide
  source 1..100: mol_type = other DNA
ccacgtaccgctcgatggccgttttagagctagaaatagcaagttaaaataaggctagtccgttatcaacttgaaaaagtggcaccgagtcggtgctttt

SEQ ID NO: 130 - DNA, 100 bp, synthetic construct
  misc_feature 1..100: note = Description of Artificial Sequence: Synthetic polynucleotide
  source 1..100: mol_type = other DNA
caccaccacgtaccgctcgagttttagagctagaaatagcaagttaaaataaggctagtccgttatcaacttgaaaaagtggcaccgagtcggtgctttt

SEQ ID NO: 131 - DNA, 100 bp, synthetic construct
  misc_feature 1..100: note = Description of Artificial Sequence: Synthetic polynucleotide
  source 1..100: mol_type = other DNA
cctggccatcgaggggtacggttttagagctagaaatagcaagttaaaataaggctagtccgttatcaacttgaaaaagtggcaccgagtcggtgctttt

SEQ ID NO: 132 - DNA, 100 bp, synthetic construct
  misc_feature 1..100: note = Description of Artificial Sequence: Synthetic polynucleotide
  source 1..100: mol_type = other DNA
ggccatcgaggggtacgtgggttttagagctagaaatagcaagttaaaataaggctagtccgttatcaacttgaaaaagtggcaccgagtcggtgctttt

SEQ ID NO: 133 - DNA, 100 bp, synthetic construct
  misc_feature 1..100: note = Description of Artificial Sequence: Synthetic polynucleotide
  source 1..100: mol_type = other DNA
ccacgtacccctcgatggccgttttagagctagaaatagcaagttaaaataaggctagtccgttatcaacttgaaaaagtggcaccgagtcggtgctttt

SEQ ID NO: 134 - DNA, 100 bp, synthetic construct
  misc_feature 1..100: note = Description of Artificial Sequence: Synthetic polynucleotide
  source 1..100: mol_type = other DNA
caccaccacgtacccctcgagttttagagctagaaatagcaagttaaaataaggctagtccgttatcaacttgaaaaagtggcaccgagtcggtgctttt

SEQ ID NO: 135 - DNA, 100 bp, synthetic construct
  misc_feature 1..100: note = Description of Artificial Sequence: Synthetic polynucleotide
  source 1..100: mol_type = other DNA
cctggccatcgagtggtacggttttagagctagaaatagcaagttaaaataaggctagtccgttatcaacttgaaaaagtggcaccgagtcggtgctttt

SEQ ID NO: 136 - DNA, 100 bp, synthetic construct
  misc_feature 1..100: note = Description of Artificial Sequence: Synthetic polynucleotide
  source 1..100: mol_type = other DNA
ggccatcgagtggtacgtgggttttagagctagaaatagcaagttaaaataaggctagtccgttatcaacttgaaaaagtggcaccgagtcggtgctttt

SEQ ID NO: 137 - DNA, 100 bp, synthetic construct
  misc_feature 1..100: note = Description of Artificial Sequence: Synthetic polynucleotide
  source 1..100: mol_type = other DNA
ccacgtaccactcgatggccgttttagagctagaaatagcaagttaaaataaggctagtccgttatcaacttgaaaaagtggcaccgagtcggtgctttt

SEQ ID NO: 138 - DNA, 100 bp, synthetic construct
  misc_feature 1..100: note = Description of Artificial Sequence: Synthetic polynucleotide
  source 1..100: mol_type = other DNA
caccaccacgtaccactcgagttttagagctagaaatagcaagttaaaataaggctagtccgttatcaacttgaaaaagtggcaccgagtcggtgctttt

SEQ ID NO: 139 - DNA, 100 bp, synthetic construct
  misc_feature 1..100: note = Description of Artificial Sequence: Synthetic polynucleotide
  source 1..100: mol_type = other DNA
cctggccatcgagctgtacggttttagagctagaaatagcaagttaaaataaggctagtccgttatcaacttgaaaaagtggcaccgagtcggtgctttt

SEQ ID NO: 140 - DNA, 100 bp, synthetic construct
  misc_feature 1..100: note = Description of Artificial Sequence: Synthetic polynucleotide
  source 1..100: mol_type = other DNA
ggccatcgagctgtacgtgggttttagagctagaaatagcaagttaaaataaggctagtccgttatcaacttgaaaaagtggcaccgagtcggtgctttt

SEQ ID NO: 141 - DNA, 100 bp, synthetic construct
  misc_feature 1..100: note = Description of Artificial Sequence: Synthetic polynucleotide
  source 1..100: mol_type = other DNA
ccacgtacagctcgatggccgttttagagctagaaatagcaagttaaaataaggctagtccgttatcaacttgaaaaagtggcaccgagtcggtgctttt

SEQ ID NO: 142 - DNA, 100 bp, synthetic construct
  misc_feature 1..100: note = Description of Artificial Sequence: Synthetic polynucleotide
  source 1..100: mol_type = other DNA
caccaccacgtacagctcgagttttagagctagaaatagcaagttaaaataaggctagtccgttatcaacttgaaaaagtggcaccgagtcggtgctttt

SEQ ID NO: 143 - DNA, 23 bp, Mus musculus
  source 1..23: mol_type = other DNA
aggtccaggatttcctcaaaggg

SEQ ID NO: 144 - DNA, 23 bp, Mus musculus
  source 1..23: mol_type = other DNA
atctagagttcgagctgatgagg

SEQ ID NO: 145 - DNA, 23 bp, Mus musculus
  source 1..23: mol_type = other DNA
gtgggcgtcaggtctttctcggg

SEQ ID NO: 146 - DNA, 23 bp, Mus musculus
  source 1..23: mol_type = other DNA
acttgaaagtgatgatgttgggg

SEQ ID NO: 147 - DNA, 23 bp, Mus musculus
  source 1..23: mol_type = other DNA
gaagaaggttcgagatctcaagg

SEQ ID NO: 148 - DNA, 23 bp, Mus musculus
  source 1..23: mol_type = other DNA
ggaggaggtagctgtgaagaagg

SEQ ID NO: 149 - DNA, 23 bp, Mus musculus
  source 1..23: mol_type = other DNA
gggacgcttccacggggaggagg

SEQ ID NO: 150 - DNA, 23 bp, Mus musculus
  source 1..23: mol_type = other DNA
gttttcctgggacgcttccacgg

SEQ ID NO: 151 - DNA, 23 bp, Mus musculus
  source 1..23: mol_type = other DNA
aggtccaggatttcctcaaaggg

SEQ ID NO: 152 - DNA, 23 bp, Mus musculus
  source 1..23: mol_type = other DNA
acgaaatgagcttgcagctatgg

SEQ ID NO: 153 - DNA, 23 bp, Mus musculus
  source 1..23: mol_type = other DNA
gtggatggagagaacacgaacgg

SEQ ID NO: 154 - DNA, 23 bp, Mus musculus
  source 1..23: mol_type = other DNA
ggcagcggcgggtttctcgaagg

SEQ ID NO: 155 - DNA, 23 bp, Mus musculus
  source 1..23: mol_type = other DNA
gggtttctcgaaggactgtttgg

SEQ ID NO: 156 - DNA, 23 bp, Mus musculus
  source 1..23: mol_type = other DNA
aggaaatctcagagctgcaatgg

SEQ ID NO: 157 - DNA, 23 bp, Mus musculus
  source 1..23: mol_type = other DNA
gcaagtgccgacctacttgaagg

SEQ ID NO: 158 - DNA, 23 bp, Mus musculus
  source 1..23: mol_type = other DNA
gacctcgtacagctgtccgtggg

SEQ ID NO: 159 - DNA, 23 bp, Mus musculus
  source 1..23: mol_type = other DNA
gcagccggggcgtgatcttccgg

SEQ ID NO: 160 - DNA, 23 bp, Mus musculus
  source 1..23: mol_type = other DNA
ggatggcaccagaggtgatccgg

SEQ ID NO: 161 - DNA, 23 bp, Mus musculus
  source 1..23: mol_type = other DNA
gatccggaatgagcctgtctcgg

SEQ ID NO: 162 - DNA, 23 bp, Mus musculus
  source 1..23: mol_type = other DNA
ggctcattccggatcacctctgg

SEQ ID NO: 163 - skipped

SEQ ID NO: 164 - DNA, 23 bp, Homo sapiens
  source 1..23: mol_type = other DNA
acaatgtcattttgtggtattgg

SEQ ID NO: 165 - DNA, 23 bp, Homo sapiens
  source 1..23: mol_type = other DNA
gttaagacaatgtcattttgtgg

SEQ ID NO: 166 - DNA, 23 bp, Homo sapiens
  source 1..23: mol_type = other DNA
aaactggattgtgagttttaagg

SEQ ID NO: 167 - DNA, 23 bp, Homo sapiens
  source 1..23: mol_type = other DNA
attctaattttgtaaaaaactgg

SEQ ID NO: 168 - DNA, 23 bp, Homo sapiens
  source 1..23: mol_type = other DNA
atcctgtgttatccaatatacgg

SEQ ID NO: 169 - DNA, 23 bp, Homo sapiens
  source 1..23: mol_type = other DNA
atattggataacacaggatttgg

SEQ ID NO: 170 - DNA, 23 bp, Homo sapiens
  source 1..23: mol_type = other DNA
ggccgtatattggataacacagg

SEQ ID NO: 171 - DNA, 23 bp, Homo sapiens
  source 1..23: mol_type = other DNA
cagaaattgaggccgtatattgg

SEQ ID NO: 172 - DNA, 23 bp, Homo sapiens
  source 1..23: mol_type = other DNA
aagctaatactcagaaattgagg

SEQ ID NO: 173 - DNA, 23 bp, Homo sapiens
  source 1..23: mol_type = other DNA
ctaaagctgtacattcattttgg

SEQ ID NO: 174 - DNA, 23 bp, Homo sapiens
  source 1..23: mol_type = other DNA
aaatgaatgtacagctttagtgg

SEQ ID NO: 175 - DNA, 23 bp, Homo sapiens
  source 1..23: mol_type = other DNA
cttgagtattctaagagttttgg

SEQ ID NO: 176 - DNA, 23 bp, Homo sapiens
  source 1..23: mol_type = other DNA
aagtaagcactagtttgtgaagg

SEQ ID NO: 177 - DNA, 23 bp, Homo sapiens
  source 1..23: mol_type = other DNA
ctcagcaactgtgtgcatgaagg

SEQ ID NO: 178 - DNA, 23 bp, Homo sapiens
  source 1..23: mol_type = other DNA
tctgtcactgatggcacactagg

SEQ ID NO: 179 - DNA, 23 bp, Homo sapiens
  source 1..23: mol_type = other DNA
acatcatcatctgtcactgatgg

SEQ ID NO: 180 - DNA, 23 bp, Homo sapiens
  source 1..23: mol_type = other DNA
tgaaaagaaatataaaattctgg

SEQ ID NO: 181 - DNA, 23 bp, Homo sapiens
  source 1..23: mol_type = other DNA
ctgaatgggcagaactctgaagg

SEQ ID NO: 182 - DNA, 23 bp, Homo sapiens
  source 1..23: mol_type = other DNA
attcagtgcctatgtgtgtgtgg

SEQ ID NO: 183 - DNA, 23 bp, Homo sapiens
  source 1..23: mol_type = other DNA
ttcagtgcctatgtgtgtgtggg

SEQ ID NO: 184 - DNA, 23 bp, Homo sapiens
  source 1..23: mol_type = other DNA
agtgcctatgtgtgtgtgggtgg

SEQ ID NO: 185 - DNA, 23 bp, Homo sapiens
  source 1..23: mol_type = other DNA
gtgcctatgtgtgtgtgggtggg

SEQ ID NO: 186 - DNA, 23 bp, Homo sapiens
  source 1..23: mol_type = other DNA
cctatgtgtgtgtgggtgggtgg

SEQ ID NO: 187 - DNA, 23 bp, Homo sapiens
  source 1..23: mol_type = other DNA
cacacacataggcactgaatggg

SEQ ID NO: 188 - DNA, 23 bp, Homo sapiens
  source 1..23: mol_type = other DNA
acacacacataggcactgaatgg

SEQ ID NO: 189 - DNA, 23 bp, Homo sapiens
  source 1..23: mol_type = other DNA
tgtgtgtgtgggtgggtggcagg

SEQ ID NO: 190 - DNA, 23 bp, Homo sapiens
  source 1..23: mol_type = other DNA
ccacccacccacacacacatagg

SEQ ID NO: 191 - DNA, 23 bp, Homo sapiens
  source 1..23: mol_type = other DNA
ggcaggatttcgccagagtgtgg

SEQ ID NO: 192 - DNA, 23 bp, Homo sapiens
  source 1..23: mol_type = other DNA
ggatttcgccagagtgtggaagg

SEQ ID NO: 193 - DNA, 23 bp, Homo sapiens
  source 1..23: mol_type = other DNA
atctgctgccttccacactctgg

SEQ ID NO: 194 - DNA, 23 bp, Homo sapiens
  source 1..23: mol_type = other DNA
tgagccagtgaataatagaaagg

SEQ ID NO: 195 - DNA, 23 bp, Homo sapiens
  source 1..23: mol_type = other DNA
tgttcctttctattattcactgg

SEQ ID NO: 196 - DNA, 23 bp, Homo sapiens
  source 1..23: mol_type = other DNA
aaaggaacatttagatagagcgg

SEQ ID NO: 197 - DNA, 23 bp, Homo sapiens
  source 1..23: mol_type = other DNA
catttagatagagcggttgaagg

SEQ ID NO: 198 - DNA, 23 bp, Homo sapiens
  source 1..23: mol_type = other DNA
cttaaatatcaattatttggtgg

SEQ ID NO: 199 - DNA, 23 bp, Homo sapiens
  source 1..23: mol_type = other DNA
gatatttaagctgaactgtgagg

SEQ ID NO: 200 - DNA, 23 bp, Homo sapiens
  source 1..23: mol_type = other DNA
cagcttaaatatcaattatttgg

SEQ ID NO: 201 - DNA, 23 bp, Homo sapiens
  source 1..23: mol_type = other DNA
agtaaacccagatgctccaaagg

SEQ ID NO: 202 - DNA, 23 bp, Homo sapiens
  source 1..23: mol_type = other DNA
ctccaaaggaagaaagcatttgg

SEQ ID NO: 203 - DNA, 23 bp, Homo sapiens
  source 1..23: mol_type = other DNA
tttcttcctttggagcatctggg

SEQ ID NO: 204 - DNA, 23 bp, Homo sapiens
  source 1..23: mol_type = other DNA
ctttcttcctttggagcatctgg

SEQ ID NO: 205 - DNA, 23 bp, Homo sapiens
  source 1..23: mol_type = other DNA
ttccaaatgctttcttcctttgg

SEQ ID NO: 206 - DNA, 23 bp, Homo sapiens
  source 1..23: mol_type = other DNA
ggaatactataagatctgagagg

SEQ ID NO: 207 - DNA, 23 bp, Homo sapiens
  source 1..23: mol_type = other DNA
gaaaaaatgattgaaatgattgg

SEQ ID NO: 208 - DNA, 23 bp, Homo sapiens
  source 1..23: mol_type = other DNA
atgattgaaatgattggagcagg

SEQ ID NO: 209 - DNA, 23 bp, Homo sapiens
  source 1..23: mol_type = other DNA
gattggagcaggaaatgatttgg

SEQ ID NO: 210 - DNA, 23 bp, Homo sapiens
  source 1..23: mol_type = other DNA
tggagcaggaaatgatttggagg

SEQ ID NO: 211 - DNA, 23 bp, Homo sapiens
  source 1..23: mol_type = other DNA
caggaaatgatttggaggactgg

SEQ ID NO: 212 - DNA, 23 bp, Homo sapiens
  source 1..23: mol_type = other DNA
tttatatactatgaatacacagg

SEQ ID NO: 213 - DNA, 23 bp, Homo sapiens
  source 1..23: mol_type = other DNA
atatactatgaatacacaggagg

SEQ ID NO: 214 - DNA, 23 bp, Homo sapiens
  source 1..23: mol_type = other DNA
tatactatgaatacacaggaggg

SEQ ID NO: 215 - DNA, 23 bp, Homo sapiens
  source 1..23: mol_type = other DNA
gaatacacaggagggaaaacagg

SEQ ID NO: 216 - DNA, 23 bp, Homo sapiens
  source 1..23: mol_type = other DNA
tacacaggagggaaaacaggagg

SEQ ID NO: 217 - DNA, 23 bp, Homo sapiens
  source 1..23: mol_type = other DNA
ggagggaaaacaggaggctgagg

SEQ ID NO: 218 - DNA, 23 bp, Homo sapiens
  source 1..23: mol_type = other DNA
gagggaaaacaggaggctgaggg

SEQ ID NO: 219 - DNA, 23 bp, Homo sapiens
  source 1..23: mol_type = other DNA
ttcctttcatcctttttcacagg

SEQ ID NO: 220 - DNA, 23 bp, Homo sapiens
  source 1..23: mol_type = other DNA
ggatgaaaggaagaaaaaaatgg

SEQ ID NO: 221 - DNA, 23 bp, Homo sapiens
  source 1..23: mol_type = other DNA
atcctgtgaaaaaggatgaaagg

SEQ ID NO: 222 - DNA, 23 bp, Homo sapiens
  source 1..23: mol_type = other DNA
ataggattatcctgtgaaaaagg

SEQ ID NO: 223 - DNA, 23 bp, Homo sapiens
  source 1..23: mol_type = other DNA
agagatttgtaacaaacaatagg

SEQ ID NO: 224 - DNA, 23 bp, Homo sapiens
  source 1..23: mol_type = other DNA
agcaaaatatcagggtaggtggg

SEQ ID NO: 225 - DNA, 23 bp, Homo sapiens
  source 1..23: mol_type = other DNA
tagcaaaatatcagggtaggtgg

SEQ ID NO: 226 - DNA, 23 bp, Homo sapiens
  source 1..23: mol_type = other DNA
aaatagcaaaatatcagggtagg

SEQ ID NO: 227 - DNA, 23 bp, Homo sapiens
  source 1..23: mol_type = other DNA
agggaaatagcaaaatatcaggg

SEQ ID NO: 228 - DNA, 23 bp, Homo sapiens
  source 1..23: mol_type = other DNA
aagggaaatagcaaaatatcagg

SEQ ID NO: 229 - DNA, 23 bp, Homo sapiens
  source 1..23: mol_type = other DNA
agagatgaaagtggtaataaggg

SEQ ID NO: 230 - DNA, 23 bp, Homo sapiens
  source 1..23: mol_type = other DNA
aagagatgaaagtggtaataagg

SEQ ID NO: 231 - DNA, 23 bp, Homo sapiens
  source 1..23: mol_type = other DNA
atgaagataagagatgaaagtgg

SEQ ID NO: 232 - DNA, 23 bp, Homo sapiens
  source 1..23: mol_type = other DNA
aagaaaatttagagttactaggg

SEQ ID NO: 233 - DNA, 23 bp, Homo sapiens
  source 1..23: mol_type = other DNA
aaagaaaatttagagttactagg

SEQ ID NO: 234 - DNA, 23 bp, Homo sapiens
  source 1..23: mol_type = other DNA
ccctcctcctgaaacttcactgg

SEQ ID NO: 235 - DNA, 23 bp, Homo sapiens
  source 1..23: mol_type = other DNA
ccagtgaagtttcaggaggaggg

SEQ ID NO: 236 - DNA, 23 bp, Homo sapiens
  source 1..23: mol_type = other DNA
accagtgaagtttcaggaggagg

SEQ ID NO: 237 - DNA, 23 bp, Homo sapiens
  source 1..23: mol_type = other DNA
gagaccagtgaagtttcaggagg

SEQ ID NO: 238 - DNA, 23 bp, Homo sapiens
  source 1..23: mol_type = other DNA
aaggagaccagtgaagtttcagg

SEQ ID NO: 239 - DNA, 23 bp, Homo sapiens
  source 1..23: mol_type = other DNA
attgaggtagaatcaagaagagg

SEQ ID NO: 240 - DNA, 23 bp, Homo sapiens
  source 1..23: mol_type = other DNA
aaagaaatagatgtagattgagg

SEQ ID NO: 241 - DNA, 23 bp, Homo sapiens
  source 1..23: mol_type = other DNA
aggaaatacaaaaactggatggg

SEQ ID NO: 242 - DNA, 23 bp, Homo sapiens
  source 1..23: mol_type = other DNA
aaggaaatacaaaaactggatgg

SEQ ID NO: 243 - DNA, 23 bp, Homo sapiens
  source 1..23: mol_type = other DNA
taataaggaaatacaaaaactgg

SEQ ID NO: 244 - DNA, 23 bp, Homo sapiens
  source 1..23: mol_type = other DNA
gaagaatggaatagataataagg

SEQ ID NO: 245 - DNA, 23 bp, Homo sapiens
  source 1..23: mol_type = other DNA
cagatttcatgtgtgaagaatgg

SEQ ID NO: 246 - DNA, 23 bp, Homo sapiens
  source 1..23: mol_type = other DNA
gtaaaggttcatgagactagagg

SEQ ID NO: 247 - DNA, 23 bp, Homo sapiens
  source 1..23: mol_type = other DNA
tgttgctttttgagaggtaaagg

SEQ ID NO: 248 - DNA, 23 bp, Homo sapiens
  source 1..23: mol_type = other DNA
aattattgttgctttttgagagg

SEQ ID NO: 249 - DNA, 23 bp, Homo sapiens
  source 1..23: mol_type = other DNA
atttgacaccacatgcactgtgg

SEQ ID NO: 250 - DNA, 23 bp, Homo sapiens
  source 1..23: mol_type = other DNA
tggtgtcaaatatggtgcttagg

SEQ ID NO: 251 - DNA, 23 bp, Homo sapiens
  source 1..23: mol_type = other DNA
agtgcatgtggtgtcaaatatgg

SEQ ID NO: 252 - DNA, 23 bp, Homo sapiens
  source 1..23: mol_type = other DNA
gtggagagccacagtgcatgtgg

SEQ ID NO: 253 - DNA, 23 bp, Homo sapiens
  source 1..23: mol_type = other DNA
aaagctaccggttacctgaaggg

SEQ ID NO: 254 - DNA, 23 bp, Homo sapiens
  source 1..23: mol_type = other DNA
aaaagctaccggttacctgaagg

SEQ ID NO: 255 - DNA, 23 bp, Homo sapiens
  source 1..23: mol_type = other DNA
gtagcttttgacagtttttaagg

SEQ ID NO: 256 - DNA, 23 bp, Homo sapiens
  source 1..23: mol_type = other DNA
gcttttgacagtttttaaggcgg

SEQ ID NO: 257 - DNA, 23 bp, Homo sapiens
  source 1..23: mol_type = other DNA
cttttgacagtttttaaggcggg

SEQ ID NO: 258 - DNA, 23 bp, Homo sapiens
  source 1..23: mol_type = other DNA
ttttgacagtttttaaggcgggg

SEQ ID NO: 259 - DNA, 23 bp, Homo sapiens
  source 1..23: mol_type = other DNA
taaaaactgtcaaaagctaccgg

SEQ ID NO: 260 - DNA, 23 bp, Homo sapiens
  source 1..23: mol_type = other DNA
ttttaaggcggggagtcacatgg

SEQ ID NO: 261 - DNA, 23 bp, Homo sapiens
  source 1..23: mol_type = other DNA
tttaaggcggggagtcacatggg

SEQ ID NO: 262 - DNA, 23 bp, Homo sapiens
  source 1..23: mol_type = other DNA
gggagtcacatgggagtcacagg

SEQ ID NO: 263 - DNA, 23 bp, Homo sapiens
  source 1..23: mol_type = other DNA
ggagtcacatgggagtcacaggg

SEQ ID NO: 264 - DNA, 23 bp, Homo sapiens
  source 1..23: mol_type = other DNA
tcacatgggagtcacagggtagg

SEQ ID NO: 265 - DNA, 23 bp, Homo sapiens
  source 1..23: mol_type = other DNA
gtttagaatgatcattcttgtgg

SEQ ID NO: 266 - DNA, 23 bp, Homo sapiens
  source 1..23: mol_type = other DNA
tgatcattcttgtggcagtaagg

SEQ ID NO: 267 - DNA, 23 bp, Homo sapiens
  source 1..23: mol_type = other DNA
tcattcttgtggcagtaaggagg

SEQ ID NO: 268 - DNA, 23 bp, Homo sapiens
  source 1..23: mol_type = other DNA
cagtaaggaggatgtaagactgg

SEQ ID NO: 269 - DNA, 23 bp, Homo sapiens
  source 1..23: mol_type = other DNA
gtaagactggagatagagacagg

SEQ ID NO: 270 - DNA, 23 bp, Homo sapiens
  source 1..23: mol_type = other DNA
ggagatagagacaggaataatgg

SEQ ID NO: 271 - DNA, 23 bp, Homo sapiens
  source 1..23: mol_type = other DNA
attttttattatctttattgtgg

SEQ ID NO: 272 - DNA, 23 bp, Homo sapiens
  source 1..23: mol_type = other DNA
aaaactaagacactgccaatagg

SEQ ID NO: 273 - DNA, 23 bp, Homo sapiens
  source 1..23: mol_type = other DNA
aaactaagacactgccaataggg

SEQ ID NO: 274 - DNA, 23 bp, Homo sapiens
  source 1..23: mol_type = other DNA
aagacactgccaatagggatagg

SEQ ID NO: 275 - DNA, 23 bp, Homo sapiens
  source 1..23: mol_type = other DNA
tatctcatacctatccctattgg

SEQ ID NO: 276 - DNA, 23 bp, Homo sapiens
  source 1..23: mol_type = other DNA
gagatattcacaattacaactgg

SEQ ID NO: 277 - DNA, 23 bp, Homo sapiens
  source 1..23: mol_type = other DNA
gaaagatgaaaaatactctttgg

SEQ ID NO: 278 - DNA, 23 bp, Homo sapiens
  source 1..23: mol_type = other DNA
aaagatgaaaaatactctttggg

SEQ ID NO: 279 - DNA, 23 bp, Homo sapiens
  source 1..23: mol_type = other DNA
ccacaagatgtctcttgcctagg

SEQ ID NO: 280 - DNA, 23 bp, Homo sapiens
  source 1..23: mol_type = other DNA
cctaggcaagagacatcttgtgg

SEQ ID NO: 281 - DNA, 23 bp, Homo sapiens
  source 1..23: mol_type = other DNA
tgcctaggactttctaatgctgg

SEQ ID NO: 282 - DNA, 23 bp, Homo sapiens
  source 1..23: mol_type = other DNA
aggactttctaatgctggagagg

SEQ ID NO: 283 - DNA, 23 bp, Homo sapiens
  source 1..23: mol_type = other DNA
tttctaatgctggagaggatagg

SEQ ID NO: 284 - DNA, 23 bp, Homo sapiens
  source 1..23: mol_type = other DNA
ctccagcattagaaagtcctagg

SEQ ID NO: 285 - DNA, 23 bp, Homo sapiens
  source 1..23: mol_type = other DNA
tgctggagaggataggacagagg

SEQ ID NO: 286 - DNA, 23 bp, Homo sapiens
  source 1..23: mol_type = other DNA
agaggataggacagaggacatgg

SEQ ID NO: 287 - DNA, 23 bp, Homo sapiens
  source 1..23: mol_type = other DNA
aggacagaggacatggagaatgg

SEQ ID NO: 288 - DNA, 23 bp, Homo sapiens
  source 1..23: mol_type = other DNA
aaattatcaccacactaaatagg

SEQ ID NO: 289 - DNA, 23 bp, Homo sapiens
  source 1..23: mol_type = other DNA
aaaattatgcctatttagtgtgg

SEQ ID NO: 290 - DNA, 23 bp, Homo sapiens
  source 1..23: mol_type = other DNA
gtaacataatcacctctctttgg

SEQ ID NO: 291 - DNA, 23 bp, Homo sapiens
  source 1..23: mol_type = other DNA
ctgctgcttttgccaaagagagg

SEQ ID NO: 292 - DNA, 23 bp, Homo sapiens
  source 1..23: mol_type = other DNA
aacatgactcataatttagtagg

SEQ ID NO: 293 - DNA, 23 bp, Homo sapiens
  source 1..23: mol_type = other DNA
cgttgttctgagtagctttcagg

SEQ ID NO: 294 - DNA, 23 bp, Homo sapiens
  source 1..23: mol_type = other DNA
attagctcaaaagcttttgctgg

SEQ ID NO: 295 - DNA, 23 bp, Homo sapiens
  source 1..23: mol_type = other DNA
tcttattctactcctgtgaaagg

SEQ ID NO: 296 - DNA, 23 bp, Homo sapiens
  source 1..23: mol_type = other DNA
cttatctaagatcctttcacagg

SEQ ID NO: 297 - DNA, 23 bp, Homo sapiens
  source 1..23: mol_type = other DNA
ataagcctctatttctgatgagg

SEQ ID NO: 298 - DNA, 23 bp, Homo sapiens
  source 1..23: mol_type = other DNA
aaatagaggcttatggattttgg

SEQ ID NO: 299 - DNA, 23 bp, Homo sapiens
  source 1..23: mol_type = other DNA
tcatcagaaatagaggcttatgg

SEQ ID NO: 300 - DNA, 23 bp, Homo sapiens
  source 1..23: mol_type = other DNA
atgaggaagatgaacaaatcagg

SEQ ID NO: 301 - DNA, 23 bp, Homo sapiens
  source 1..23: mol_type = other DNA
atcttcctcatcagaaatagagg

SEQ ID NO: 302 - DNA, 23 bp, Homo sapiens
  source 1..23: mol_type = other DNA
cttgttctgtcctcagtaaaagg

SEQ ID NO: 303 - DNA, 23 bp, Homo sapiens
  source 1..23: mol_type = other DNA
taaaaggtatagagttcaagtgg

SEQ ID NO: 304 - DNA, 23 bp, Homo sapiens
  source 1..23: mol_type = other DNA
gaactctataccttttactgagg

SEQ ID NO: 305 - DNA, 23 bp, Homo sapiens
  source 1..23: mol_type = other DNA
tggctgtaagataactacaaagg

SEQ ID NO: 306 - DNA, 23 bp, Homo sapiens
  source 1..23: mol_type = other DNA
ggctgtaagataactacaaaggg

SEQ ID NO: 307 - DNA, 23 bp, Homo sapiens
  source 1..23: mol_type = other DNA
taagataactacaaagggtctgg

SEQ ID NO: 308 - DNA, 23 bp, Homo sapiens
  source 1..23: mol_type = other DNA
taagttacaatctgtgaatatgg

SEQ ID NO: 309 - DNA, 23 bp, Homo sapiens
  source 1..23: mol_type = other DNA
gaatttacagagtgcatccatgg

SEQ ID NO: 310 - DNA, 23 bp, Homo sapiens
  source 1..23: mol_type = other DNA
acagagtgcatccatggtccagg

SEQ ID NO: 311 - DNA, 23 bp, Homo sapiens
  source 1..23: mol_type = other DNA
taagggctcttcctggaccatgg

SEQ ID NO: 312 - DNA, 23 bp, Homo sapiens
  source 1..23: mol_type = other DNA
cttaatatttcagctactgttgg

SEQ ID NO: 313 - DNA, 23 bp, Homo sapiens
  source 1..23: mol_type = other DNA
gaaatattaagggctcttcctgg

SEQ ID NO: 314 - DNA, 23 bp, Homo sapiens
  source 1..23: mol_type = other DNA
aacagtagctgaaatattaaggg

SEQ ID NO: 315 - DNA, 23 bp, Homo sapiens
  source 1..23: mol_type = other DNA
caacagtagctgaaatattaagg

SEQ ID NO: 316 - DNA, 23 bp, Homo sapiens
  source 1..23: mol_type = other DNA
tgttggctacatccattccaagg

SEQ ID NO: 317 - DNA, 23 bp, Homo sapiens
  source 1..23: mol_type = other DNA
cattccaaggaacaaaagccagg

SEQ ID NO: 318 - DNA, 23 bp, Homo sapiens
  source 1..23: mol_type = other DNA
attccaaggaacaaaagccaggg

SEQ ID NO: 319 - DNA, 23 bp, Homo sapiens
  source 1..23: mol_type = other DNA
ggaacaaaagccagggaccatgg

SEQ ID NO: 320 - DNA, 23 bp, Homo sapiens
  source 1..23: mol_type = other DNA
gaacaaaagccagggaccatggg

SEQ ID NO: 321 - DNA, 23 bp, Homo sapiens
  source 1..23: mol_type = other DNA
ctggcttttgttccttggaatgg

SEQ ID NO: 322 - DNA, 23 bp, Homo sapiens
  source 1..23: mol_type = other DNA
ggtccctggcttttgttccttgg

SEQ ID NO: 323 - DNA, 23 bp, Homo sapiens
  source 1..23: mol_type = other DNA
catgggagaatagtttgttctgg

SEQ ID NO: 324 - DNA, 23 bp, Homo sapiens
  source 1..23: mol_type = other DNA
atgggagaatagtttgttctggg

SEQ ID NO: 325 - DNA, 23 bp, Homo sapiens
  source 1..23: mol_type = other DNA
aactattctcccatggtccctgg

SEQ ID NO: 326 - DNA, 23 bp, Homo sapiens
  source 1..23: mol_type = other DNA
gaatagtttgttctgggtacagg

SEQ ID NO: 327 - DNA, 23 bp, Homo sapiens
  source 1..23: mol_type = other DNA
aatagtttgttctgggtacaggg

SEQ ID NO: 328 - DNA, 23 bp, Homo sapiens
  source 1..23: mol_type = other DNA
atagtttgttctgggtacagggg

SEQ ID NO: 329 - DNA, 23 bp, Homo sapiens
  source 1..23: mol_type = other DNA
cagaacaaactattctcccatgg

SEQ ID NO: 330 - DNA, 23 bp, Homo sapiens
  source 1..23: mol_type = other DNA
tgggtacaggggtaagagaaagg

SEQ ID NO: 331 - DNA, 23 bp, Homo sapiens
  source 1..23: mol_type = other DNA
gggtacaggggtaagagaaaggg

SEQ ID NO: 332 - DNA, 23 bp, Homo sapiens
  source 1..23: mol_type = other DNA
acaggggtaagagaaagggatgg

SEQ ID NO: 333 - DNA, 23 bp, Homo sapiens
  source 1..23: mol_type = other DNA
caggggtaagagaaagggatggg

SEQ ID NO: 334 - DNA, 23 bp, Homo sapiens
  source 1..23: mol_type = other DNA
agaatattgtaatcaaaggaggg

SEQ ID NO: 335 - DNA, 23 bp, Homo sapiens
  source 1..23: mol_type = other DNA
tagaatattgtaatcaaaggagg

SEQ ID NO: 336 - DNA, 23 bp, Homo sapiens
  source 1..23: mol_type = other DNA
aggtagaatattgtaatcaaagg

SEQ ID NO: 337 - DNA, 23 bp, Homo sapiens
  source 1..23: mol_type = other DNA
tcctttttctcttagatgtctgg

SEQ ID NO: 338 - DNA, 23 bp, Homo sapiens
  source 1..23: mol_type = other DNA
gagaaaaaggagcatgaaacagg

SEQ ID NO: 339 - DNA, 23 bp, Homo sapiens
  source 1..23: mol_type = other DNA
cttagatgtctggttaaaagagg

SEQ ID NO: 340 - DNA, 23 bp, Homo sapiens
  source 1..23: mol_type = other DNA
accagacatctaagagaaaaagg

SEQ ID NO: 341 - DNA, 23 bp, Homo sapiens
  source 1..23: mol_type = other DNA
taaatcatgcaagtgacctaagg

SEQ ID NO: 342 - DNA, 23 bp, Homo sapiens
  source 1..23: mol_type = other DNA
ttatctagatcattctccttagg

SEQ ID NO: 343 - DNA, 23 bp, Homo sapiens
  source 1..23: mol_type = other DNA
aatatattatctatttatagtgg

SEQ ID NO: 344 - DNA, 23 bp, Homo sapiens
  source 1..23: mol_type = other DNA
gtgtgtgtgtgtgtgttatgtgg

SEQ ID NO: 345 - DNA, 23 bp, Homo sapiens
  source 1..23: mol_type = other DNA
tgtgtgtgtgtgtgttatgtggg

SEQ ID NO: 346 - DNA, 23 bp, Homo sapiens
  source 1..23: mol_type = other DNA
actaaactgaatgaataatatgg

SEQ ID NO: 347 - DNA, 23 bp, Homo sapiens
  source 1..23: mol_type = other DNA
tgttaaatttatataaatgcagg

SEQ ID NO: 348 - DNA, 23 bp, Homo sapiens
  source 1..23: mol_type = other DNA
tttttgtgtttggggggatttgg

SEQ ID NO: 349 - DNA, 23 bp, Homo sapiens
  source 1..23: mol_type = other DNA
acaaaaatattgaagaaaaatgg

SEQ ID NO: 350 - DNA, 23 bp, Homo sapiens
  source 1..23: mol_type = other DNA
tcaatatttttgtgtttgggggg

SEQ ID NO: 351 - DNA, 23 bp, Homo sapiens
  source 1..23: mol_type = other DNA
ttcaatatttttgtgtttggggg

SEQ ID NO: 352 - DNA, 23 bp, Homo sapiens
  source 1..23: mol_type = other DNA
cttcaatatttttgtgtttgggg

SEQ ID NO: 353 - DNA, 23 bp, Homo sapiens
  source 1..23: mol_type = other DNA
tcttcaatatttttgtgtttggg

SEQ ID NO: 354 - DNA, 23 bp, Homo sapiens
  source 1..23: mol_type = other DNA
ttcttcaatatttttgtgtttgg

SEQ ID NO: 355 - DNA, 23 bp, Homo sapiens
  source 1..23: mol_type = other DNA
gaaaaatggctaatttcacaagg

SEQ ID NO: 356 - DNA, 23 bp, Homo sapiens
  source 1..23: mol_type = other DNA
aatcacacaaacttacctccagg

SEQ ID NO: 357 - DNA, 27 bp, Homo sapiens
  source 1..27: mol_type = other DNA
atgtcattttgtggtattggtctgaat

SEQ ID NO: 358 - DNA, 27 bp, Homo sapiens
  source 1..27: mol_type = other DNA
caatccagttttttacaaaattagaat

SEQ ID NO: 359 - DNA, 27 bp, Homo sapiens
  source 1..27: mol_type = other DNA
aattttgtaaaaaactggattgtgagt

SEQ ID NO: 360 - DNA, 27 bp, Homo sapiens
  source 1..27: mol_type = other DNA
aaattctaattttgtaaaaaactggat

SEQ ID NO: 361 - DNA, 27 bp, Homo sapiens
  source 1..27: mol_type = other DNA
tccaatatacggcctcaatttctgagt

SEQ ID NO: 362 - DNA, 27 bp, Homo sapiens
  source 1..27: mol_type = other DNA
gtatattggataacacaggatttggat

SEQ ID NO: 363 - DNA, 27 bp, Homo sapiens
  source 1..27: mol_type = other DNA
gaggccgtatattggataacacaggat

SEQ ID NO: 364 - DNA, 27 bp, Homo sapiens
  source 1..27: mol_type = other DNA
ctcagaaattgaggccgtatattggat

SEQ ID NO: 365 - DNA, 27 bp, Homo sapiens
  source 1..27: mol_type = other DNA
attcattttggccaaaactcttagaat

SEQ ID NO: 366 - DNA, 27 bp, Homo sapiens
  source 1..27: mol_type = other DNA
gccaaaactcttagaatactcaagagt

SEQ ID NO: 367 - DNA, 27 bp, Homo sapiens
  source 1..27: mol_type = other DNA
attctaagagttttggccaaaatgaat

SEQ ID NO: 368 - DNA, 27 bp, Homo sapiens
  source 1..27: mol_type = other DNA
tgcaaaactcttgagtattctaagagt

SEQ ID NO: 369 - DNA, 27 bp, Homo sapiens
  source 1..27: mol_type = other DNA
tttgtgaaggctgcaaaactcttgagt

SEQ ID NO: 370 - DNA, 27 bp, Homo sapiens
  source 1..27: mol_type = other DNA
catctgtcactgatggcacactaggat

SEQ ID NO: 371 - DNA, 27 bp, Homo sapiens
  source 1..27: mol_type = other DNA
gaaatataaaattctggccttcagagt

SEQ ID NO: 372 - DNA, 27 bp, Homo sapiens
  source 1..27: mol_type = other DNA
ccattcagtgcctatgtgtgtgtgggt

SEQ ID NO: 373 - DNA, 27 bp, Homo sapiens
  source 1..27: mol_type = other DNA
aatgggcagaactctgaaggccagaat

SEQ ID NO: 374 - DNA, 27 bp, Homo sapiens
  source 1..27: mol_type = other DNA
tcagtgcctatgtgtgtgtgggtgggt

SEQ ID NO: 375 - DNA, 27 bp, Homo sapiens
  source 1..27: mol_type = other DNA
tatgtgtgtgtgggtgggtggcaggat

SEQ ID NO: 376 - DNA, 27 bp, Homo sapiens
  source 1..27: mol_type = other DNA
gggtgggtggcaggatttcgccagagt

SEQ ID NO: 377 - DNA, 27 bp, Homo sapiens
  source 1..27: mol_type = other DNA
ccacccacacacacataggcactgaat

SEQ ID NO: 378 - DNA, 27 bp, Homo sapiens
  source 1..27: mol_type = other DNA
catttagaaagaaaataaaaacagagt

SEQ ID NO: 379 - DNA, 27 bp, Homo sapiens
  source 1..27: mol_type = other DNA
taaaaacagagtactgagccagtgaat

SEQ ID NO: 380 - DNA, 27 bp, Homo sapiens
  source 1..27: mol_type = other DNA
aacatttagatagagcggttgaaggat

SEQ ID NO: 381 - DNA, 27 bp, Homo sapiens
  source 1..27: mol_type = other DNA
gctccaaaggaagaaagcatttggaat

SEQ ID NO: 382 - DNA, 27 bp, Homo sapiens
  source 1..27: mol_type = other DNA
tgctttcttcctttggagcatctgggt

SEQ ID NO: 383 - DNA, 27 bp, Homo sapiens
  source 1..27: mol_type = other DNA
ataattatagttttatatactatgaat

SEQ ID NO: 384 - DNA, 27 bp, Homo sapiens
  source 1..27: mol_type = other DNA
caggagggaaaacaggaggctgagggt

SEQ ID NO: 385 - DNA, 27 bp, Homo sapiens
  source 1..27: mol_type = other DNA
tcttcctttcatcctttttcacaggat

SEQ ID NO: 386 - DNA, 27 bp, Homo sapiens
  source 1..27: mol_type = other DNA
aaggatgaaaggaagaaaaaaatggat

SEQ ID NO: 387 - DNA, 27 bp, Homo sapiens
  source 1..27: mol_type = other DNA
caataggattatcctgtgaaaaaggat

SEQ ID NO: 388 - DNA, 27 bp, Homo sapiens
  source 1..27: mol_type = other DNA
aaagagatttgtaacaaacaataggat

SEQ ID NO: 389 - DNA, 27 bp, Homo sapiens
  source 1..27: mol_type = other DNA
ataagggaaatagcaaaatatcagggt

SEQ ID NO: 390 - DNA, 27 bp, Homo sapiens
  source 1..27: mol_type = other DNA
aaaatttagagttactagggagagagt

SEQ ID NO: 391 - DNA, 27 bp, Homo sapiens
  source 1..27: mol_type = other DNA
caggaggagggaaagaaaatttagagt

SEQ ID NO: 392 - DNA, 27 bp, Homo sapiens
  source 1..27: mol_type = other DNA
agaaatagatgtagattgaggtagaat

SEQ ID NO: 393 - DNA, 27 bp, Homo sapiens
  source 1..27: mol_type = other DNA
ataaggaaatacaaaaactggatgggt

SEQ ID NO: 394 - DNA, 27 bp, Homo sapiens
  source 1..27: mol_type = other DNA
gataataaggaaatacaaaaactggat

SEQ ID NO: 395 - DNA, 27 bp, Homo sapiens
  source 1..27: mol_type = other DNA
tcagatttcatgtgtgaagaatggaat

SEQ ID NO: 396 - DNA, 27 bp, Homo sapiens
  source 1..27: mol_type = other DNA
gaaaatcagatttcatgtgtgaagaat

SEQ ID NO: 397 - DNA, 27 bp, Homo sapiens
  source 1..27: mol_type = other DNA
cttttgacagtttttaaggcggggagt

SEQ ID NO: 398 - DNA, 27 bp, Homo sapiens
  source 1..27: mol_type = other DNA
tcaaaagctaccggttacctgaagggt

SEQ ID NO: 399 - DNA, 27 bp, Homo sapiens
  source 1..27: mol_type = other DNA
ttttaaggcggggagtcacatgggagt

SEQ ID NO: 400 - DNA, 27 bp, Homo sapiens
  source 1..27: mol_type = other DNA
cggggagtcacatgggagtcacagggt

SEQ ID NO: 401 - DNA, 27 bp, Homo sapiens
  source 1..27: mol_type = other DNA
agtcacatgggagtcacagggtaggat

SEQ ID NO: 402 - DNA, 27 bp, Homo sapiens
  source 1..27: mol_type = other DNA
tcacagggtaggattcatgtttagaat

SEQ ID NO: 403 - DNA, 27 bp, Homo sapiens
  source 1..27: mol_type = other DNA
gatcattcttgtggcagtaaggaggat

SEQ ID NO: 404 - DNA, 27 bp, Homo sapiens
  source 1..27: mol_type = other DNA
acaagaatgatcattctaaacatgaat

SEQ ID NO: 405 - DNA, 27 bp, Homo sapiens
  source 1..27: mol_type = other DNA
tgtaagactggagatagagacaggaat

SEQ ID NO: 406 - DNA, 27 bp, Homo sapiens
  source 1..27: mol_type = other DNA
ttacatcctccttactgccacaagaat

SEQ ID NO: 407 - DNA, 27 bp, Homo sapiens
  source 1..27: mol_type = other DNA
taaaactaagacactgccaatagggat

SEQ ID NO: 408 - DNA, 27 bp, Homo sapiens
  source 1..27: mol_type = other DNA
tttaacgttatcattttcccaaagagt

SEQ ID NO: 409 - DNA, 27 bp, Homo sapiens
  source 1..27: mol_type = other DNA
catcttgtggataatgtatcaatgagt

SEQ ID NO: 410 - DNA, 27 bp, Homo sapiens
  source 1..27: mol_type = other DNA
ctaggactttctaatgctggagaggat

SEQ ID NO: 411 - DNA, 27 bp, Homo sapiens
  source 1..27: mol_type = other DNA
gtcctaggcaagagacatcttgtggat

SEQ ID NO: 412 - DNA, 27 bp, Homo sapiens
  source 1..27: mol_type = other DNA
gaggataggacagaggacatggagaat

SEQ ID NO: 413 - DNA, 27 bp, Homo sapiens
  source 1..27: mol_type = other DNA
aaacatgactcataatttagtaggaat

SEQ ID NO: 414 - DNA, 27 bp, Homo sapiens
  source 1..27: mol_type = other DNA
tttcaggattcctactaaattatgagt

SEQ ID NO: 415 - DNA, 27 bp, Homo sapiens
  source 1..27: mol_type = other DNA
tcagaacaacgttttcatttactgaat

SEQ ID NO: 416 - DNA, 27 bp, Homo sapiens
  source 1..27: mol_type = other DNA
aacgttgttctgagtagctttcaggat

SEQ ID NO: 417 - DNA, 27 bp, Homo sapiens
  source 1..27: mol_type = other DNA
tcagtaaatgaaaacgttgttctgagt

SEQ ID NO: 418 - DNA, 27 bp, Homo sapiens
  source 1..27: mol_type = other DNA
cagcaaaagcttttgagctaatagaat

SEQ ID NO: 419 - DNA, 27 bp, Homo sapiens
  source 1..27: mol_type = other DNA
gatcttattctactcctgtgaaaggat

SEQ ID NO: 420 - DNA, 27 bp, Homo sapiens
  source 1..27: mol_type = other DNA
tcctgtgaaaggatcttagataagaat

SEQ ID NO: 421 - DNA, 27 bp, Homo sapiens
  source 1..27: mol_type = other DNA
tctaagatcctttcacaggagtagaat

SEQ ID NO: 422 - DNA, 27 bp, Homo sapiens
  source 1..27: mol_type = other DNA
tcttatctaagatcctttcacaggagt

SEQ ID NO: 423 - DNA, 27 bp, Homo sapiens
  source 1..27: mol_type = other DNA
agaaatagaggcttatggattttggat

SEQ ID NO: 424 - DNA, 27 bp, Homo sapiens
  source 1..27: mol_type = other DNA
cctcatcagaaatagaggcttatggat

SEQ ID NO: 425 - DNA, 27 bp, Homo sapiens
  source 1..27: mol_type = other DNA
ttctgtcctcagtaaaaggtatagagt

SEQ ID NO: 426 - DNA, 27 bp, Homo sapiens
  source 1..27: mol_type = other DNA
agtggctgtaagataactacaaagggt

SEQ ID NO: 427 - DNA, 27 bp, Homo sapiens
  source 1..27: mol_type = other DNA
gaatatcataagttacaatctgtgaat

SEQ ID NO: 428 - DNA, 27 bp, Homo sapiens
  source 1..27: mol_type = other DNA
tattctacaataaaaaatgatgagaat

SEQ ID NO: 429 - DNA, 27 bp, Homo sapiens
  source 1..27: mol_type = other DNA
ataaaaaatgatgagaatttacagagt

SEQ ID NO: 430 - DNA, 27 bp, Homo sapiens
  source 1..27: mol_type = other DNA
ttttttattgtagaataaatgtagaat

SEQ ID NO: 431 - DNA, 27 bp, Homo sapiens
  source 1..27: mol_type = other DNA
aattctcatcattttttattgtagaat

SEQ ID NO: 432 - DNA, 27 bp, Homo sapiens
  source 1..27: mol_type = other DNA
attaagggctcttcctggaccatggat

SEQ ID NO: 433 - DNA, 27 bp, Homo sapiens
  source 1..27: mol_type = other DNA
aacaaaagccagggaccatgggagaat

SEQ ID NO: 434 - DNA, 27 bp, Homo sapiens
  source 1..27: mol_type = other DNA
ccctggcttttgttccttggaatggat

SEQ ID NO: 435 - DNA, 27 bp, Homo sapiens
  source 1..27: mol_type = other DNA
accatgggagaatagtttgttctgggt

SEQ ID NO: 436 - DNA, 27 bp, Homo sapiens
  source 1..27: mol_type = other DNA
tggtccctggcttttgttccttggaat

SEQ ID NO: 437 - DNA, 27 bp, Homo sapiens
  source 1..27: mol_type = other DNA
agaatagtttgttctgggtacaggggt

SEQ ID NO: 438 - DNA, 27 bp, Homo sapiens
  source 1..27: mol_type = other DNA
ctgggtacaggggtaagagaaagggat

SEQ ID NO: 439 - DNA, 27 bp, Homo sapiens
  source 1..27: mol_type = other DNA
agagaaagggatgggcacttaatgagt

SEQ ID NO: 440 - DNA, 27 bp, Homo sapiens
  source 1..27: mol_type = other DNA
gaaaaaggagcatgaaacaggtagaat

SEQ ID NO: 441 - DNA, 27 bp, Homo sapiens
  source 1..27: mol_type = other DNA
aaatcatgcaagtgacctaaggagaat

SEQ ID NO: 442 - DNA, 27 bp, Homo sapiens
  source 1..27: mol_type = other DNA
taggtcacttgcatgatttagctgaat

SEQ ID NO: 443 - DNA, 27 bp, Homo sapiens
  source 1..27: mol_type = other DNA
tttatagtggctgaatgacttctgaat

SEQ ID NO: 444 - DNA, 27 bp, Homo sapiens
  source 1..27: mol_type = other DNA
tatattatctatttatagtggctgaat

SEQ ID NO: 445 - DNA, 27 bp, Homo sapiens
  source 1..27: mol_type = other DNA
aacaaaatatttataactaaactgaat

SEQ ID NO: 446 - DNA, 27 bp, Homo sapiens
  source 1..27: mol_type = other DNA
aaatatttataactaaactgaatgaat

SEQ ID NO: 447 - DNA, 27 bp, Homo sapiens
  source 1..27: mol_type = other DNA
taactaaactgaatgaataatatggat

SEQ ID NO: 448 - DNA, 27 bp, Homo sapiens
  source 1..27: mol_type = other DNA
cttcaatatttttgtgtttggggggat

SEQ ID NO: 449 - DNA, 27 bp, Homo sapiens
  source 1..27: mol_type = other DNA
atggctaatttcacaaggttcaagaat

SEQ ID NO: 450 - DNA, 25 bp, Homo sapiens
  source 1..25: mol_type = other DNA
atccagttttttacaaaattagaat

SEQ ID NO: 451 - DNA, 25 bp, Homo sapiens
  source 1..25: mol_type = other DNA
agtggcagaagctaatactcagaaa

SEQ ID NO: 452 - DNA, 25 bp, Homo sapiens
  source 1..25: mol_type = other DNA
tcattttggccaaaactcttagaat

SEQ ID NO: 453 - DNA, 25 bp, Homo sapiens
  source 1..25: mol_type = other DNA
tgatgatgtttacagtgaaaagaaa

SEQ ID NO: 454 - DNA, 25 bp, Homo sapiens
  source 1..25: mol_type = other DNA
tgggcagaactctgaaggccagaat

SEQ ID NO: 455 - DNA, 25 bp, Homo sapiens
  source 1..25: mol_type = other DNA
gtactgagccagtgaataatagaaa

SEQ ID NO: 456 - DNA, 25 bp, Homo sapiens
  source 1..25: mol_type = other DNA
aacccagatgctccaaaggaagaaa

SEQ ID NO: 457 - DNA, 25 bp, Homo sapiens
  source 1..25: mol_type = other DNA
aaaggaagaaaaaaatggatagaaa

SEQ ID NO: 458 - DNA, 25 bp, Homo sapiens
  source 1..25: mol_type = other DNA
tgtgaaaaaggatgaaaggaagaaa

SEQ ID NO: 459 - DNA, 25 bp, Homo sapiens
  source 1..25: mol_type = other DNA
aaaatatcagggtaggtgggagaaa

SEQ ID NO: 460 - DNA, 25 bp, Homo sapiens
  source 1..25: mol_type = other DNA
gaagtttcaggaggagggaaagaaa

SEQ ID NO: 461 - DNA, 25 bp, Homo sapiens
  source 1..25: mol_type = other DNA
aaatagatgtagattgaggtagaat

SEQ ID NO: 462 - DNA, 25 bp, Homo sapiens
  source 1..25: mol_type = other DNA
atttgatagtagaagaaaaaagaaa

SEQ ID NO: 463 - DNA, 25 bp, Homo sapiens
  source 1..25: mol_type = other DNA
acttgaaatttgatagtagaagaaa

SEQ ID NO: 464 - DNA, 25 bp, Homo sapiens
  source 1..25: mol_type = other DNA
tggatgggtaataaagcaaaagaaa

SEQ ID NO: 465 - DNA, 25 bp, Homo sapiens
  source 1..25: mol_type = other DNA
aaatcagatttcatgtgtgaagaat

SEQ ID NO: 466 - DNA, 25 bp, Homo sapiens
  source 1..25: mol_type = other DNA
acagggtaggattcatgtttagaat

SEQ ID NO: 467 - DNA, 25 bp, Homo sapiens
  source 1..25: mol_type = other DNA
acatcctccttactgccacaagaat

SEQ ID NO: 468 - DNA, 25 bp, Homo sapiens
  source 1..25: mol_type = other DNA
ggataggacagaggacatggagaat

SEQ ID NO: 469 - DNA, 25 bp, Homo sapiens
  source 1..25: mol_type = other DNA
gtcctatcctctccagcattagaaa

SEQ ID NO: 470 - DNA, 25 bp, Homo sapiens
  source 1..25: mol_type = other DNA
gtgattatgttactttttatagaaa

SEQ ID NO: 471 - DNA, 25 bp, Homo sapiens
  source 1..25: mol_type = other DNA
ctctctttggcaaaagcagcagaaa

SEQ ID NO: 472 - DNA, 25 bp, Homo sapiens
  source 1..25: mol_type = other DNA
gcaaaagcttttgagctaatagaat

SEQ ID NO: 473 - DNA, 25 bp, Homo sapiens
  source 1..25: mol_type = other DNA
ctgtgaaaggatcttagataagaat

SEQ ID NO: 474 - DNA, 25 bp, Homo sapiens
  source 1..25: mol_type = other DNA
taagatcctttcacaggagtagaat

SEQ ID NO: 475 - DNA, 25 bp, Homo sapiens
  source 1..25: mol_type = other DNA
atttgttcatcttcctcatcagaaa

SEQ ID NO: 476 - DNA, 25 bp, Homo sapiens
  source 1..25: mol_type = other DNA
ttctacaataaaaaatgatgagaat

SEQ ID NO: 477 - DNA, 25 bp, Homo sapiens
  source 1..25: mol_type = other DNA
ttttattgtagaataaatgtagaat

SEQ ID NO: 478 - DNA, 25 bp, Homo sapiens
  source 1..25: mol_type = other DNA
ttctcatcattttttattgtagaat

SEQ ID NO: 479 - DNA, 25 bp, Homo sapiens
  source 1..25: mol_type = other DNA
caaaagccagggaccatgggagaat

SEQ ID NO: 480 - DNA, 25 bp, Homo sapiens
  source 1..25: mol_type = other DNA
gttctgggtacaggggtaagagaaa

SEQ ID NO: 481 - DNA, 25 bp, Homo sapiens
  source 1..25: mol_type = other DNA
aaaaggagcatgaaacaggtagaat

SEQ ID NO: 482 - DNA, 25 bp, Homo sapiens
  source 1..25: mol_type = other DNA
cttttaaccagacatctaagagaaa

SEQ ID NO: 483 - DNA, 25 bp, Homo sapiens
  source 1..25: mol_type = other DNA
atcatgcaagtgacctaaggagaat

SEQ ID NO: 484 - DNA, 25 bp, Homo sapiens
  source 1..25: mol_type = other DNA
cccaaacacaaaaatattgaagaaa

SEQ ID NO: 485 - DNA, 25 bp, Homo sapiens
  source 1..25: mol_type = other DNA
ggctaatttcacaaggttcaagaat

SEQ ID NO: 486 - DNA, 32 bp, Homo sapiens
  source 1..32: mol_type = other DNA
gaggaaattctaattttgtaaaaaactggatt

SEQ ID NO: 487 - DNA, 32 bp, Homo sapiens
  source 1..32: mol_type = other DNA
aattgaggccgtatattggataacacaggatt

SEQ ID NO: 488 - DNA, 32 bp, Homo sapiens
  source 1..32: mol_type = other DNA
tgcctatgtgtgtgtgggtgggtggcaggatt

SEQ ID NO: 489 - DNA, 32 bp, Homo sapiens
  source 1..32: mol_type = other DNA
aatactataagatctgagaggaaaaaatgatt

SEQ ID NO: 490 - DNA, 32 bp, Homo sapiens
  source 1..32: mol_type = other DNA
agatctgagaggaaaaaatgattgaaatgatt

SEQ ID NO: 491 - DNA, 32 bp, Homo sapiens
  source 1..32: mol_type = other DNA
aatgattgaaatgattggagcaggaaatgatt

SEQ ID NO: 492 - DNA, 32 bp, Homo sapiens
  source 1..32: mol_type = other DNA
agaaaaagagatttgtaacaaacaataggatt

SEQ ID NO: 493 - DNA, 32 bp, Homo sapiens
  source 1..32: mol_type = other DNA
aaatatcagggtaggtgggagaaaaagagatt

SEQ ID NO: 494 - DNA, 32 bp, Homo sapiens
  source 1..32: mol_type = other DNA
atagtagaagaaaaaagaaatagatgtagatt

SEQ ID NO: 495 - DNA, 32 bp, Homo sapiens
  source 1..32: mol_type = other DNA
tctattccattcttcacacatgaaatctgatt

SEQ ID NO: 496 - DNA, 32 bp, Homo sapiens
  source 1..32: mol_type = other DNA
ttcatgagactagaggtcacgaaaatcagatt

SEQ ID NO: 497 - DNA, 32 bp, Homo sapiens
  source 1..32: mol_type = other DNA
ggggagtcacatgggagtcacagggtaggatt

SEQ ID NO: 498 - DNA, 32 bp, Homo sapiens
  source 1..32: mol_type = other DNA
ctttggcaaaagcagcagaaagcaaactgatt

SEQ ID NO: 499 - DNA, 32 bp, Homo sapiens
  source 1..32: mol_type = other DNA
caaaagcagcagaaagcaaactgattatgatt

SEQ ID NO: 500 - DNA, 32 bp, Homo sapiens
  source 1..32: mol_type = other DNA
ctttctgctgcttttgccaaagagaggtgatt

SEQ ID NO: 501 - DNA, 32 bp, Homo sapiens
  source 1..32: mol_type = other DNA
tgaaaacgttgttctgagtagctttcaggatt

SEQ ID NO: 502 - DNA, 32 bp, Homo sapiens
  source 1..32: mol_type = other DNA
catcagaaatagaggcttatggattttggatt

SEQ ID NO: 503 - DNA, 32 bp, Homo sapiens
  source 1..32: mol_type = other DNA
tcttcctcatcagaaatagaggcttatggatt

SEQ ID NO: 504 - DNA, 32 bp, Homo sapiens
  source 1..32: mol_type = other DNA
ccttttactgaggacagaacaagctcctgatt

SEQ ID NO: 505 - DNA, 32 bp, Homo sapiens
  source 1..32: mol_type = other DNA
actacaaagggtctggtccatattcacagatt

SEQ ID NO: 506 - DNA, 32 bp, Homo sapiens
  source 1..32: mol_type = other DNA
gggcacttaatgagtgcttccctcctttgatt

SEQ ID NO: 507 - DNA, 32 bp, Homo sapiens
  source 1..32: mol_type = other DNA
ctcttagatgtctggttaaaagaggtttgatt

SEQ ID NO: 508 - DNA, 32 bp, Homo sapiens
  source 1..32: mol_type = other DNA
tagatcattctccttaggtcacttgcatgatt

SEQ ID NO: 509 - DNA, 32 bp, Homo sapiens
  source 1..32: mol_type = other DNA
atttatagtggctgaatgacttctgaatgatt

SEQ ID NO: 510 - DNA, 32 bp, Homo sapiens
  source 1..32: mol_type = other DNA
atatttttgtgtttggggggatttggcagatt

SEQ ID NO: 511 - DNA, 32 bp, Homo sapiens
  source 1..32: mol_type = other DNA
ttttcttcaatatttttgtgtttggggggatt

SEQ ID NO: 512 - DNA, 24 bp, Homo sapiens
  source 1..24: mol_type = other DNA
tctgctgccttccacactctggcg

SEQ ID NO: 513 - DNA, 24 bp, Homo sapiens
  source 1..24: mol_type = other DNA
agaaaggaacatttagatagagcg

SEQ ID NO: 514 - DNA, 24 bp, Homo sapiens
  source 1..24: mol_type = other DNA
tagcttttgacagtttttaaggcg

SEQ ID NO: 515 - skipped

SEQ ID NO: 516 - DNA, 27 bp, Homo sapiens
  source 1..27: mol_type = other DNA
tttcttagtgatatttttcctttattc

SEQ ID NO: 517 - DNA, 27 bp, Homo sapiens
  source 1..27: mol_type = other DNA
tttgtggtattggtctgaattacaatg

SEQ ID NO: 518 - DNA, 27 bp, Homo sapiens
  source 1..27: mol_type = other DNA
ttttgtggtattggtctgaattacaat

SEQ ID NO: 519 - DNA, 27 bp, Homo sapiens
  source 1..27: mol_type = other DNA
tttatataacatctccttaaaactcac

SEQ ID NO: 520 - DNA, 27 bp, Homo sapiens
  source 1..27: mol_type = other DNA
tttaaggagatgttatataaagttaag

SEQ ID NO: 521 - DNA, 27 bp, Homo sapiens
  source 1..27: mol_type = other DNA
ttttaaggagatgttatataaagttaa

SEQ ID NO: 522 - DNA, 27 bp, Homo sapiens
  source 1..27: mol_type = other DNA
tttgtaaaaaactggattgtgagtttt

SEQ ID NO: 523 - DNA, 27 bp, Homo sapiens
  source 1..27: mol_type = other DNA
ttttgtaaaaaactggattgtgagttt

SEQ ID NO: 524 - DNA, 27 bp, Homo sapiens
  source 1..27: mol_type = other DNA
tttctgagtattagcttctgccactaa

SEQ ID NO: 525 - DNA, 27 bp, Homo sapiens
  source 1..27: mol_type = other DNA
tttagtggcagaagctaatactcagaa

SEQ ID NO: 526 - DNA, 27 bp, Homo sapiens
  source 1..27: mol_type = other DNA
ttttggccaaaactcttagaatactca

SEQ ID NO: 527 - DNA, 27 bp, Homo sapiens
  source 1..27: mol_type = other DNA
tttggccaaaactcttagaatactcaa

SEQ ID NO: 528 - DNA, 27 bp, Homo sapiens
  source 1..27: mol_type = other DNA
tttggccaaaatgaatgtacagcttta

SEQ ID NO: 529 - DNA, 27 bp, Homo sapiens
  source 1..27: mol_type = other DNA
ttttggccaaaatgaatgtacagcttt

SEQ ID NO: 530 - DNA, 27 bp, Homo sapiens
  source 1..27: mol_type = other DNA
ttttgcagccttcacaaactagtgctt

SEQ ID NO: 531 - DNA, 27 bp, Homo sapiens
  source 1..27: mol_type = other DNA
tttgcagccttcacaaactagtgctta

SEQ ID NO: 532 - DNA, 27 bp, Homo sapiens
  source 1..27: mol_type = other DNA
tttgtgaaggctgcaaaactcttgagt

SEQ ID NO: 533 - DNA, 27 bp, Homo sapiens
  source 1..27: mol_type = other DNA
tttacagtgaaaagaaatataaaattc

SEQ ID NO: 534 - DNA, 27 bp, Homo sapiens
  source 1..27: mol_type = other DNA
tttcactgtaaacatcatcatctgtca

SEQ ID NO: 535 - DNA, 27 bp, Homo sapiens
  source 1..27: mol_type = other DNA
ttttcactgtaaacatcatcatctgtc

SEQ ID NO: 536 - DNA, 27 bp, Homo sapiens
  source 1..27: mol_type = other DNA
tttcttttcactgtaaacatcatcatc

SEQ ID NO: 537 - DNA, 27 bp, Homo sapiens
  source 1..27: mol_type = other DNA
tttatatttcttttcactgtaaacatc

SEQ ID NO: 538 - DNA, 27 bp, Homo sapiens
  source 1..27: mol_type = other DNA
ttttatatttcttttcactgtaaacat

SEQ ID NO: 539 - DNA, 27 bp, Homo sapiens
  source 1..27: mol_type = other DNA
tttcgccagagtgtggaaggcagcaga

SEQ ID NO: 540 - DNA, 27 bp, Homo sapiens
  source 1..27: mol_type = other DNA
tttacttgcttattatctgctgccttc

SEQ ID NO: 541 - DNA, 27 bp, Homo sapiens
  source 1..27: mol_type = other DNA
tttgtttacttgcttattatctgctgc

SEQ ID NO: 542 - DNA, 27 bp, Homo sapiens
  source 1..27: mol_type = other DNA
tttgtttgtttacttgcttattatctg

SEQ ID NO: 543 - DNA, 27 bp, Homo sapiens
  source 1..27: mol_type = other DNA
ttttgtttgtttacttgcttattatct

SEQ ID NO: 544 - DNA, 27 bp, Homo sapiens
  source 1..27: mol_type = other DNA
tttttgtttgtttacttgcttattatc

SEQ ID NO: 545 - DNA, 27 bp, Homo sapiens
  source 1..27: mol_type = other DNA
tttagtagactttttgtttgtttactt

SEQ ID NO: 546 - DNA, 27 bp, Homo sapiens
  source 1..27: mol_type = other DNA
ttttagtagactttttgtttgtttact

SEQ ID NO: 547 - DNA, 27 bp, Homo sapiens
  source 1..27: mol_type = other DNA
tttttagtagactttttgtttgtttac

SEQ ID NO: 548 - DNA, 27 bp, Homo sapiens
  source 1..27: mol_type = other DNA
tttagaaagaaaataaaaacagagtac

SEQ ID NO: 549 - DNA, 27 bp, Homo sapiens
  source 1..27: mol_type = other DNA
tttctattattcactggctcagtactc

SEQ ID NO: 550 - DNA, 27 bp, Homo sapiens
  source 1..27: mol_type = other DNA
tttagatagagcggttgaaggatgtct

SEQ ID NO: 551 - DNA, 27 bp, Homo sapiens
  source 1..27: mol_type = other DNA
tttggtggttaagacatccttcaaccg

SEQ ID NO: 552 - DNA, 27 bp, Homo sapiens
  source 1..27: mol_type = other DNA
tttaagctgaactgtgaggtacgaagt

SEQ ID NO: 553 - DNA, 27 bp, Homo sapiens
  source 1..27: mol_type = other DNA
tttattagtaaacccagatgctccaaa

SEQ ID NO: 554 - DNA, 27 bp, Homo sapiens
  source 1..27: mol_type = other DNA
tttactaataaatacttcgtacctcac

SEQ ID NO: 555 - DNA, 27 bp, Homo sapiens
  source 1..27: mol_type = other DNA
tttggagcatctgggtttactaataaa

SEQ ID NO: 556 - DNA, 27 bp, Homo sapiens
  source 1..27: mol_type = other DNA
tttcttcctttggagcatctgggttta

SEQ ID NO: 557 - DNA, 27 bp, Homo sapiens
  source 1..27: mol_type = other DNA
tttggaatactataagatctgagagga

SEQ ID NO: 558 - DNA, 27 bp, Homo sapiens
  source 1..27: mol_type = other DNA
tttcctctcagatcttatagtattcca

SEQ ID NO: 559 - DNA, 27 bp, Homo sapiens
  source 1..27: mol_type = other DNA
ttttcctctcagatcttatagtattcc

SEQ ID NO: 560 - DNA, 27 bp, Homo sapiens
  source 1..27: mol_type = other DNA
tttttcctctcagatcttatagtattc

SEQ ID NO: 561 - DNA, 27 bp, Homo sapiens
  source 1..27: mol_type = other DNA
ttttttcctctcagatcttatagtatt

SEQ ID NO: 562 - DNA, 27 bp, Homo sapiens
  source 1..27: mol_type = other DNA
tttcaatcattttttcctctcagatct

SEQ ID NO: 563 - DNA, 27 bp, Homo sapiens
  source 1..27: mol_type = other DNA
tttcctgctccaatcatttcaatcatt

SEQ ID NO: 564 - DNA, 27 bp, Homo sapiens
  source 1..27: mol_type = other DNA
ttttttaagtaaagtacataattatag

SEQ ID NO: 565 - DNA, 27 bp, Homo sapiens
  source 1..27: mol_type = other DNA
tttttaagtaaagtacataattatagt

SEQ ID NO: 566 - DNA, 27 bp, Homo sapiens
  source 1..27: mol_type = other DNA
ttttaagtaaagtacataattatagtt

SEQ ID NO: 567 - DNA, 27 bp, Homo sapiens
  source 1..27: mol_type = other DNA
tttaagtaaagtacataattatagttt

SEQ ID NO: 568 - DNA, 27 bp, Homo sapiens
  source 1..27: mol_type = other DNA
ttttatatactatgaatacacaggagg

SEQ ID NO: 569 - DNA, 27 bp, Homo sapiens
  source 1..27: mol_type = other DNA
tttatatactatgaatacacaggaggg

SEQ ID NO: 570 - DNA, 27 bp, Homo sapiens
  source 1..27: mol_type = other DNA
tttccctcctgtgtattcatagtatat

SEQ ID NO: 571 - DNA, 27 bp, Homo sapiens
  source 1..27: mol_type = other DNA
ttttccctcctgtgtattcatagtata

SEQ ID NO: 572 - DNA, 27 bp, Homo sapiens
  source 1..27: mol_type = other DNA
ttttaaatatctttctatccatttttt

SEQ ID NO: 573 - DNA, 27 bp, Homo sapiens
  source 1..27: mol_type = other DNA
tttaaatatctttctatccattttttt

SEQ ID NO: 574 - DNA, 27 bp, Homo sapiens
  source 1..27: mol_type = other DNA
tttaaaacaccctcagcctcctgtttt

SEQ ID NO: 575 - DNA, 27 bp, Homo sapiens
  source 1..27: mol_type = other DNA
tttctatccatttttttcttcctttca

SEQ ID NO: 576 - DNA, 27 bp, Homo sapiens
  source 1..27: mol_type = other DNA
tttttttcttcctttcatcctttttca

SEQ ID NO: 577 - DNA, 27 bp, Homo sapiens
  source 1..27: mol_type = other DNA
ttttttcttcctttcatcctttttcac

SEQ ID NO: 578 - DNA, 27 bp, Homo sapiens
  source 1..27: mol_type = other DNA
tttttcttcctttcatcctttttcaca

SEQ ID NO: 579 - DNA, 27 bp, Homo sapiens
  source 1..27: mol_type = other DNA
ttttcttcctttcatcctttttcacag

SEQ ID NO: 580 - DNA, 27 bp, Homo sapiens
  source 1..27: mol_type = other DNA
tttcttcctttcatcctttttcacagg

SEQ ID NO: 581 - DNA, 27 bp, Homo sapiens
  source 1..27: mol_type = other DNA
tttcatcctttttcacaggataatcct

SEQ ID NO: 582 - DNA, 27 bp, Homo sapiens
  source 1..27: mol_type = other DNA
tttttcacaggataatcctattgtttg

SEQ ID NO: 583 - DNA, 27 bp, Homo sapiens
  source 1..27: mol_type = other DNA
ttttcacaggataatcctattgtttgt

SEQ ID NO: 584 - DNA, 27 bp, Homo sapiens
  source 1..27: mol_type = other DNA
tttcacaggataatcctattgtttgtt

SEQ ID NO: 585 - DNA, 27 bp, Homo sapiens
  source 1..27: mol_type = other DNA
tttgttacaaatctctttttctcccac

SEQ ID NO: 586 - DNA, 27 bp, Homo sapiens
  source 1..27: mol_type = other DNA
tttgtaacaaacaataggattatcctg

SEQ ID NO: 587 - DNA, 27 bp, Homo sapiens
  source 1..27: mol_type = other DNA
tttttctcccacctaccctgatatttt

SEQ ID NO: 588 - DNA, 27 bp, Homo sapiens
  source 1..27: mol_type = other DNA
ttttctcccacctaccctgatattttg

SEQ ID NO: 589 - DNA, 27 bp, Homo sapiens
  source 1..27: mol_type = other DNA
tttctcccacctaccctgatattttgc

SEQ ID NO: 590 - DNA, 27 bp, Homo sapiens
  source 1..27: mol_type = other DNA
ttttgctatttcccttattaccacttt

SEQ ID NO: 591 - DNA, 27 bp, Homo sapiens
  source 1..27: mol_type = other DNA
tttgctatttcccttattaccactttc

SEQ ID NO: 592 - DNA, 27 bp, Homo sapiens
  source 1..27: mol_type = other DNA
tttcccttattaccactttcatctctt

SEQ ID NO: 593 - DNA, 27 bp, Homo sapiens
  source 1..27: mol_type = other DNA
tttcatctcttatcttcatcactctct

SEQ ID NO: 594 - DNA, 27 bp, Homo sapiens
  source 1..27: mol_type = other DNA
tttagagttactagggagagagtgatg

SEQ ID NO: 595 - DNA, 27 bp, Homo sapiens
  source 1..27: mol_type = other DNA
ttttctttccctcctcctgaaacttca

SEQ ID NO: 596 - DNA, 27 bp, Homo sapiens
  source 1..27: mol_type = other DNA
tttctttccctcctcctgaaacttcac

SEQ ID NO: 597 - DNA, 27 bp, Homo sapiens
  source 1..27: mol_type = other DNA
tttccctcctcctgaaacttcactggt

SEQ ID NO: 598 - DNA, 27 bp, Homo sapiens
  source 1..27: mol_type = other DNA
tttcaggaggagggaaagaaaatttag

SEQ ID NO: 599 - DNA, 27 bp, Homo sapiens
  source 1..27: mol_type = other DNA
tttaaaggagaccagtgaagtttcagg

SEQ ID NO: 600 - DNA, 27 bp, Homo sapiens
  source 1..27: mol_type = other DNA
ttttaaaggagaccagtgaagtttcag

SEQ ID NO: 601 - DNA, 27 bp, Homo sapiens
  source 1..27: mol_type = other DNA
tttttaaaggagaccagtgaagtttca

SEQ ID NO: 602 - DNA, 27 bp, Homo sapiens
  source 1..27: mol_type = other DNA
ttttttaaaggagaccagtgaagtttc

SEQ ID NO: 603 - DNA, 27 bp, Homo sapiens
  source 1..27: mol_type = other DNA
tttttttaaaggagaccagtgaagttt

SEQ ID NO: 604 - DNA, 27 bp, Homo sapiens
  source 1..27: mol_type = other DNA
ttttttttaaaggagaccagtgaagtt

SEQ ID NO: 605 - DNA, 27 bp, Homo sapiens
  source 1..27: mol_type = other DNA
tttttttttaaaggagaccagtgaagt

SEQ ID NO: 606 - DNA, 27 bp, Homo sapiens
  source 1..27: mol_type = other DNA
ttttttttttaaaggagaccagtgaag

SEQ ID NO: 607 - DNA, 27 bp, Homo sapiens
  source 1..27: mol_type = other DNA
tttttttttttaaaggagaccagtgaa

SEQ ID NO: 608 - DNA, 27 bp, Homo sapiens
  source 1..27: mol_type = other DNA
ttttttttttttaaaggagaccagtga

SEQ ID NO: 609 - DNA, 27 bp, Homo sapiens
  source 1..27: mol_type = other DNA
tttttttttttttaaaggagaccagtg

SEQ ID NO: 610 - DNA, 27 bp, Homo sapiens
  source 1..27: mol_type = other DNA
tttcttttttcttctactatcaaattt

SEQ ID NO: 611 - DNA, 27 bp, Homo sapiens
  source 1..27: mol_type = other DNA
ttttttcttctactatcaaatttcaag

SEQ ID NO: 612 - DNA, 27 bp, Homo sapiens
  source 1..27: mol_type = other DNA
tttttcttctactatcaaatttcaagt

SEQ ID NO: 613 - DNA, 27 bp, Homo sapiens
  source 1..27: mol_type = other DNA
ttttcttctactatcaaatttcaagtt

SEQ ID NO: 614 - DNA, 27 bp, Homo sapiens
  source 1..27: mol_type = other DNA
tttcttctactatcaaatttcaagttt

SEQ ID NO: 615 - DNA, 27 bp, Homo sapiens
  source 1..27: mol_type = other DNA
tttgatagtagaagaaaaaagaaatag

SEQ ID NO: 616 - DNA, 27 bp, Homo sapiens
  source 1..27: mol_type = other DNA
tttcaagtttttcttttgctttattac

SEQ ID NO: 617 - DNA, 27 bp, Homo sapiens
  source 1..27: mol_type = other DNA
tttttcttttgctttattacccatcca

SEQ ID NO: 618 - DNA, 27 bp, Homo sapiens
  source 1..27: mol_type = other DNA
ttttcttttgctttattacccatccag

SEQ ID NO: 619 - DNA, 27 bp, Homo sapiens
  source 1..27: mol_type = other DNA
tttcttttgctttattacccatccagt

SEQ ID NO: 620 - DNA, 27 bp, Homo sapiens
  source 1..27: mol_type = other DNA
ttttgctttattacccatccagttttt

SEQ ID NO: 621 - DNA, 27 bp, Homo sapiens
  source 1..27: mol_type = other DNA
tttgctttattacccatccagtttttg

SEQ ID NO: 622 - DNA, 27 bp, Homo sapiens
  source 1..27: mol_type = other DNA
tttattacccatccagtttttgtattt

SEQ ID NO: 623 - DNA, 27 bp, Homo sapiens
  source 1..27: mol_type = other DNA
tttttgtatttccttattatctattcc

SEQ ID NO: 624 - DNA, 27 bp, Homo sapiens
  source 1..27: mol_type = other DNA
ttttgtatttccttattatctattcca

SEQ ID NO: 625 - DNA, 27 bp, Homo sapiens
  source 1..27: mol_type = other DNA
tttgtatttccttattatctattccat

SEQ ID NO: 626 - DNA, 27 bp, Homo sapiens
  source 1..27: mol_type = other DNA
tttccttattatctattccattcttca

SEQ ID NO: 627 - DNA, 27 bp, Homo sapiens
  source 1..27: mol_type = other DNA
tttcatgtgtgaagaatggaatagata

SEQ ID NO: 628 - DNA, 27 bp, Homo sapiens
  source 1..27: mol_type = other DNA
ttttcgtgacctctagtctcatgaacc

SEQ ID NO: 629 - DNA, 27 bp, Homo sapiens
  source 1..27: mol_type = other DNA
tttcgtgacctctagtctcatgaacct

SEQ ID NO: 630 - DNA, 27 bp, Homo sapiens
  source 1..27: mol_type = other DNA
tttacctctcaaaaagcaacaataatt

SEQ ID NO: 631 - DNA, 27 bp, Homo sapiens
  source 1..27: mol_type = other DNA
tttgagaggtaaaggttcatgagacta

SEQ ID NO: 632 - DNA, 27 bp, Homo sapiens
  source 1..27: mol_type = other DNA
ttttgagaggtaaaggttcatgagact

SEQ ID NO: 633 - DNA, 27 bp, Homo sapiens
  source 1..27: mol_type = other DNA
tttttgagaggtaaaggttcatgagac

SEQ ID NO: 634 - DNA, 27 bp, Homo sapiens
  source 1..27: mol_type = other DNA
tttcctaagcaccatatttgacaccac

SEQ ID NO: 635 - DNA, 27 bp, Homo sapiens
  source 1..27: mol_type = other DNA
tttgacaccacatgcactgtggctctc

SEQ ID NO: 636 - DNA, 27 bp, Homo sapiens
  source 1..27: mol_type = other DNA
ttttacccttcaggtaaccggtagctt

SEQ ID NO: 637 - DNA, 27 bp, Homo sapiens
  source 1..27: mol_type = other DNA
tttacccttcaggtaaccggtagcttt

SEQ ID NO: 638 - DNA, 27 bp, Homo sapiens
  source 1..27: mol_type = other DNA
ttttgacagtttttaaggcggggagtc

SEQ ID NO: 639 - DNA, 27 bp, Homo sapiens
  source 1..27: mol_type = other DNA
tttgacagtttttaaggcggggagtca

SEQ ID NO: 640 - DNA, 27 bp, Homo sapiens
  source 1..27: mol_type = other DNA
tttttaaggcggggagtcacatgggag

SEQ ID NO: 641 - DNA, 27 bp, Homo sapiens
  source 1..27: mol_type = other DNA
ttttaaggcggggagtcacatgggagt

SEQ ID NO: 642 - DNA, 27 bp, Homo sapiens
  source 1..27: mol_type = other DNA
tttaaggcggggagtcacatgggagtc

SEQ ID NO: 643 - DNA, 27 bp, Homo sapiens
  source 1..27: mol_type = other DNA
tttagaatgatcattcttgtggcagta

SEQ ID NO: 644 - DNA, 27 bp, Homo sapiens
  source 1..27: mol_type = other DNA
tttattgtggcagccattattcctgtc

SEQ ID NO: 645 - DNA, 27 bp, Homo sapiens
  source 1..27: mol_type = other DNA
tttattatctttattgtggcagccatt

SEQ ID NO: 646 - DNA, 27 bp, Homo sapiens
  source 1..27: mol_type = other DNA
ttttattatctttattgtggcagccat

SEQ ID NO: 647 - DNA, 27 bp, Homo sapiens
  source 1..27: mol_type = other DNA
tttttattatctttattgtggcagcca

SEQ ID NO: 648 - DNA, 27 bp, Homo sapiens
  source 1..27: mol_type = other DNA
ttttttattatctttattgtggcagcc

SEQ ID NO: 649 - DNA, 27 bp, Homo sapiens
  source 1..27: mol_type = other DNA
tttattttttattatctttattgtggc

SEQ ID NO: 650 - DNA, 27 bp, Homo sapiens
  source 1..27: mol_type = other DNA
ttttattttttattatctttattgtgg

SEQ ID NO: 651 - DNA, 27 bp, Homo sapiens
  source 1..27: mol_type = other DNA
tttaggaaagatgaaaaatactctttg

SEQ ID NO: 652 - DNA, 27 bp, Homo sapiens
  source 1..27: mol_type = other DNA
tttgggaaaatgataacgttaaacatc

SEQ ID NO: 653 - DNA, 27 bp, Homo sapiens
  source 1..27: mol_type = other DNA
tttcccaaagagtatttttcatctttc

SEQ ID NO: 654 - DNA, 27 bp, Homo sapiens
  source 1..27: mol_type = other DNA
ttttcccaaagagtatttttcatcttt

SEQ ID NO: 655 - DNA, 27 bp, Homo sapiens
  source 1..27: mol_type = other DNA
tttaacgttatcattttcccaaagagt

SEQ ID NO: 656 - DNA, 27 bp, Homo sapiens
  source 1..27: mol_type = other DNA
tttctaatgctggagaggataggacag

SEQ ID NO: 657 - DNA, 27 bp, Homo sapiens
  source 1..27: mol_type = other DNA
tttccattctccatgtcctctgtccta

SEQ ID NO: 658 - DNA, 27 bp, Homo sapiens
  source 1..27: mol_type = other DNA
ttttccattctccatgtcctctgtcct

SEQ ID NO: 659 - DNA, 27 bp, Homo sapiens
  source 1..27: mol_type = other DNA
tttttccattctccatgtcctctgtcc

SEQ ID NO: 660 - DNA, 27 bp, Homo sapiens
  source 1..27: mol_type = other DNA
ttttttccattctccatgtcctctgtc

SEQ ID NO: 661 - DNA, 27 bp, Homo sapiens
  source 1..27: mol_type = other DNA
tttttttccattctccatgtcctctgt

SEQ ID NO: 662 - DNA, 27 bp, Homo sapiens
  source 1..27: mol_type = other DNA
tttatttttttccattctccatgtcct

SEQ ID NO: 663 - DNA, 27 bp, Homo sapiens
  source 1..27: mol_type = other DNA
tttagtgtggtgataatttattttttt

SEQ ID NO: 664 - DNA, 27 bp, Homo sapiens
  source 1..27: mol_type = other DNA
ttttctataaaaagtaacataatcacc

SEQ ID NO: 665 - DNA, 27 bp, Homo sapiens
  source 1..27: mol_type = other DNA
tttctataaaaagtaacataatcacct

SEQ ID NO: 666 - DNA, 27 bp, Homo sapiens
  source 1..27: mol_type = other DNA
tttatagaaaattatgcctatttagtg

SEQ ID NO: 667 - DNA, 27 bp, Homo sapiens
  source 1..27: mol_type = other DNA
ttttatagaaaattatgcctatttagt

SEQ ID NO: 668 - DNA, 27 bp, Homo sapiens
  source 1..27: mol_type = other DNA
tttttatagaaaattatgcctatttag

SEQ ID NO: 669 - DNA, 27 bp, Homo sapiens
  source 1..27: mol_type = other DNA
tttggcaaaagcagcagaaagcaaact

SEQ ID NO: 670 - DNA, 27 bp, Homo sapiens
  source 1..27: mol_type = other DNA
tttgccaaagagaggtgattatgttac

SEQ ID NO: 671 - DNA, 27 bp, Homo sapiens
  source 1..27: mol_type = other DNA
ttttgccaaagagaggtgattatgtta

SEQ ID NO: 672 - DNA, 27 bp, Homo sapiens
  source 1..27: mol_type = other DNA
tttctgctgcttttgccaaagagaggt

SEQ ID NO: 673 - DNA, 27 bp, Homo sapiens
  source 1..27: mol_type = other DNA
tttgctttctgctgcttttgccaaaga

SEQ ID NO: 674 - DNA, 27 bp, Homo sapiens
  source 1..27: mol_type = other DNA
tttagaagtaatcataatcagtttgct

SEQ ID NO: 675 - DNA, 27 bp, Homo sapiens
  source 1..27: mol_type = other DNA
tttatcaatattatttagaagtaatca

SEQ ID NO: 676 - DNA, 27 bp, Homo sapiens
  source 1..27: mol_type = other DNA
tttagtaggaatcctgaaagctactca

SEQ ID NO: 677 - DNA, 27 bp, Homo sapiens
  source 1..27: mol_type = other DNA
tttcaggattcctactaaattatgagt

SEQ ID NO: 678 - DNA, 27 bp, Homo sapiens
  source 1..27: mol_type = other DNA
ttttcatttactgaatgtgtctctatg

SEQ ID NO: 679 - DNA, 27 bp, Homo sapiens
  source 1..27: mol_type = other DNA
tttcatttactgaatgtgtctctatga

SEQ ID NO: 680 - DNA, 27 bp, Homo sapiens
  source 1..27: mol_type = other DNA
tttactgaatgtgtctctatgagccag

SEQ ID NO: 681 - DNA, 27 bp, Homo sapiens
  source 1..27: mol_type = other DNA
tttgctggctcatagagacacattcag

SEQ ID NO: 682 - DNA, 27 bp, Homo sapiens
  source 1..27: mol_type = other DNA
ttttgctggctcatagagacacattca

SEQ ID NO: 683 - DNA, 27 bp, Homo sapiens
  source 1..27: mol_type = other DNA
ttttgagctaatagaattagatcttat

SEQ ID NO: 684 - DNA, 27 bp, Homo sapiens
  source 1..27: mol_type = other DNA
tttgagctaatagaattagatcttatt

SEQ ID NO: 685 - DNA, 27 bp, Homo sapiens
  source 1..27: mol_type = other DNA
tttcacaggagtagaataagatctaat

SEQ ID NO: 686 - DNA, 27 bp, Homo sapiens
  source 1..27: mol_type = other DNA
tttggattattcttatctaagatcctt

SEQ ID NO: 687 - DNA, 27 bp, Homo sapiens
  source 1..27: mol_type = other DNA
ttttggattattcttatctaagatcct

SEQ ID NO: 688 - DNA, 27 bp, Homo sapiens
  source 1..27: mol_type = other DNA
tttctgatgaggaagatgaacaaatca

SEQ ID NO: 689 - DNA, 27 bp, Homo sapiens
  source 1..27: mol_type = other DNA
tttgttcatcttcctcatcagaaatag

SEQ ID NO: 690 - DNA, 27 bp, Homo sapiens
  source 1..27: mol_type = other DNA
tttactgaggacagaacaagctcctga

SEQ ID NO: 691 - DNA, 27 bp, Homo sapiens
  source 1..27: mol_type = other DNA
ttttactgaggacagaacaagctcctg

SEQ ID NO: 692 - DNA, 27 bp, Homo sapiens
  source 1..27: mol_type = other DNA
tttgtagttatcttacagccacttgaa

SEQ ID NO: 693 - DNA, 27 bp, Homo sapiens
  source 1..27: mol_type = other DNA
tttattctacaataaaaaatgatgaga

SEQ ID NO: 694 - DNA, 27 bp, Homo sapiens
  source 1..27: mol_type = other DNA
tttattgtagaataaatgtagaatttt

SEQ ID NO: 695 - DNA, 27 bp, Homo sapiens
  source 1..27: mol_type = other DNA
ttttattgtagaataaatgtagaattt

SEQ ID NO: 696 - DNA, 27 bp, Homo sapiens
  source 1..27: mol_type = other DNA
tttttattgtagaataaatgtagaatt

SEQ ID NO: 697 - DNA, 27 bp, Homo sapiens
  source 1..27: mol_type = other DNA
ttttttattgtagaataaatgtagaat

SEQ ID NO: 698 - DNA, 27 bp, Homo sapiens
  source 1..27: mol_type = other DNA
tttacagagtgcatccatggtccagga

SEQ ID NO: 699 - DNA, 27 bp, Homo sapiens
  source 1..27: mol_type = other DNA
tttcagctactgttggctacatccatt

SEQ ID NO: 700 - DNA, 27 bp, Homo sapiens
  source 1..27: mol_type = other DNA
tttgttccttggaatggatgtagccaa

SEQ ID NO: 701 - DNA, 27 bp, Homo sapiens
  source 1..27: mol_type = other DNA
ttttgttccttggaatggatgtagcca

SEQ ID NO: 702 - DNA, 27 bp, Homo sapiens
  source 1..27: mol_type = other DNA
tttgttctgggtacaggggtaagagaa

SEQ ID NO: 703 - DNA, 27 bp, Homo sapiens
  source 1..27: mol_type = other DNA
tttctcttacccctgtacccagaacaa

SEQ ID NO: 704 - DNA, 27 bp, Homo sapiens
  source 1..27: mol_type = other DNA
tttgattacaatattctacctgtttca

SEQ ID NO: 705 - DNA, 27 bp, Homo sapiens
  source 1..27: mol_type = other DNA
tttcatgctcctttttctcttagatgt

SEQ ID NO: 706 - DNA, 27 bp, Homo sapiens
  source 1..27: mol_type = other DNA
tttttctcttagatgtctggttaaaag

SEQ ID NO: 707 - DNA, 27 bp, Homo sapiens
  source 1..27: mol_type = other DNA
ttttctcttagatgtctggttaaaaga

SEQ ID NO: 708 - DNA, 27 bp, Homo sapiens
  source 1..27: mol_type = other DNA
tttctcttagatgtctggttaaaagag

SEQ ID NO: 709 - DNA, 27 bp, Homo sapiens
  source 1..27: mol_type = other DNA
tttaaccagacatctaagagaaaaagg

SEQ ID NO: 710 - DNA, 27 bp, Homo sapiens
  source 1..27: mol_type = other DNA
ttttaaccagacatctaagagaaaaag

SEQ ID NO: 711 - DNA, 27 bp, Homo sapiens
  source 1..27: mol_type = other DNA
tttgattcagctaaatcatgcaagtga

SEQ ID NO: 712 - DNA, 27 bp, Homo sapiens
  source 1..27: mol_type = other DNA
tttagctgaatcaaacctcttttaacc

SEQ ID NO: 713 - DNA, 27 bp, Homo sapiens
  source 1..27: mol_type = other DNA
tttatagtggctgaatgacttctgaat

SEQ ID NO: 714 - DNA, 27 bp, Homo sapiens
  source 1..27: mol_type = other DNA
tttgttcccacataacacacacacaca

SEQ ID NO: 715 - DNA, 27 bp, Homo sapiens
  source 1..27: mol_type = other DNA
ttttgttcccacataacacacacacac

SEQ ID NO: 716 - DNA, 27 bp, Homo sapiens
  source 1..27: mol_type = other DNA
tttataactaaactgaatgaataatat

SEQ ID NO: 717 - DNA, 27 bp, Homo sapiens
  source 1..27: mol_type = other DNA
tttagttataaatattttgttcccaca

SEQ ID NO: 718 - DNA, 27 bp, Homo sapiens
  source 1..27: mol_type = other DNA
tttaacagagatataattatccatatt

SEQ ID NO: 719 - DNA, 27 bp, Homo sapiens
  source 1..27: mol_type = other DNA
tttatataaatgcaggcaaactttaat

SEQ ID NO: 720 - DNA, 27 bp, Homo sapiens
  source 1..27: mol_type = other DNA
tttatataaatttaacagagatataat

SEQ ID NO: 721 - DNA, 27 bp, Homo sapiens
  source 1..27: mol_type = other DNA
tttgcctgcatttatataaatttaaca

SEQ ID NO: 722 - DNA, 27 bp, Homo sapiens
  source 1..27: mol_type = other DNA
tttaattaaaatctgccaaatcccccc

SEQ ID NO: 723 - DNA, 27 bp, Homo sapiens
  source 1..27: mol_type = other DNA
tttaattaaagtttgcctgcatttata

SEQ ID NO: 724 - DNA, 27 bp, Homo sapiens
  source 1..27: mol_type = other DNA
ttttaattaaagtttgcctgcatttat

SEQ ID NO: 725 - DNA, 27 bp, Homo sapiens
  source 1..27: mol_type = other DNA
tttggcagattttaattaaagtttgcc

SEQ ID NO: 726 - DNA, 27 bp, Homo sapiens
  source 1..27: mol_type = other DNA
tttggggggatttggcagattttaatt

SEQ ID NO: 727 - DNA, 27 bp, Homo sapiens
  source 1..27: mol_type = other DNA
tttgtgtttggggggatttggcagatt

SEQ ID NO: 728 - DNA, 27 bp, Homo sapiens
  source 1..27: mol_type = other DNA
ttttgtgtttggggggatttggcagat

SEQ ID NO: 729 - DNA, 27 bp, Homo sapiens
  source 1..27: mol_type = other DNA
tttttgtgtttggggggatttggcaga

SEQ ID NO: 730 - DNA, 27 bp, Homo sapiens
  source 1..27: mol_type = other DNA
tttcttcaatatttttgtgtttggggg

SEQ ID NO: 731 - DNA, 27 bp, Homo sapiens
  source 1..27: mol_type = other DNA
ttttcttcaatatttttgtgtttgggg

SEQ ID NO: 732 - DNA, 27 bp, Homo sapiens
  source 1..27: mol_type = other DNA
tttttcttcaatatttttgtgtttggg

SEQ ID NO: 733 - DNA, 27 bp, Homo sapiens
  source 1..27: mol_type = other DNA
tttcacaaggttcaagaatcacacaaa

SEQ ID NO: 734 - DNA, 27 bp, Homo sapiens
  source 1..27: mol_type = other DNA
tttgtgtgattcttgaaccttgtgaaa

SEQ ID NO: 735 - DNA, 27 bp, Homo sapiens
  source 1..27: mol_type = other DNA
cacctggccccagttgtaattgtgagt

SEQ ID NO: 736 - DNA, 23 bp, Homo sapiens
  source 1..23: mol_type = other DNA
cctggccccagttgtaattgtga

SEQ ID NO: 737 - DNA, 24 bp, Homo sapiens
  source 1..24: mol_type = other DNA
cctggccccagttgtaattgtgag

SEQ ID NO: 738 - DNA, 23 bp, Homo sapiens
  source 1..23: mol_type = other DNA
gagatactcacaattacaactgg

SEQ ID NO: 739 - DNA, 230 bp, Homo sapiens
  source 1..230: mol_type = other DNA
ggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactttttccc

SEQ ID NO: 740 - DNA, 233 bp, unidentified
  misc_feature 1..233: note = Description of Unknown: SRPRPS29 bidirectional promotoer sequence
  source 1..233: mol_type = other DNA
cttgctctcagcagtgcaacgaggtaaaaggaagaagctggcccacgcatgcgctcttcaaatttttgagacagtttacccagaatgcagtgctcaaaggaaacgcgtgcgcagtgtggtcaggttgtttcgctgggtgagtaaaatgaaatcttagaggcgttgtgggctggcccagttgatgacgtcaccataccacagcttctagtgctattctgcgccggtatccgacc

SEQ ID NO: 741 - DNA, 239 bp, unidentified
  misc_feature 1..239: note = Description of Unknown: 7sk1GSTA4 bidirectional promotoer sequence
  source 1..239: mol_type = other DNA
agtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagacttccttcaggtttatatagcttgtgcgccgcttgggtacctc

SEQ ID NO: 742 - DNA, 308 bp, unidentified
  misc_feature 1..308: note = Description of Unknown: SNARG1 CGB1 bidirectional promotoer sequence
  source 1..308: mol_type = other DNA
gtctctctcttagcgggatatcttccgcaagcactgggaatgtggacatggaaagtaaattgagtctccgtgggggagtgagacagggagtgaggggtgttggacgcggcacgggaacctggccagagtcagcggacccaattggctgctctctctcagatgcagttccccttcctccctccagggggcgccacggaacgcagggccctcactggccctggggactgggtgacgtcagggatgagcctcttgtgattggctccatcaccctgcgtaagatcaaagggaagaaaggatgggcccgacaa

SEQ ID NO: 743 - DNA, 308 bp, unidentified
  misc_feature 1..308: note = Description of Unknown: SNAR CGB2 bidirectional promotoer sequence
  source 1..308: mol_type = other DNA
gtctctctcttagcgggatatcttccgcaagcactggggatgtggacatggaaagtaaattgagtctccgtgggggagtgagacagggagtgaggggtgttggacgcggcacgggaacccggccggagtcagcggacccaattggctgctctctctcagatacagttccccttcctccctccagggggcgccacggaacgcagggccctcactggccctggggactgggtgacgtcaggggtgagcctctcctgattggctccatcaccctgcgtaaggtcaaaaggaagaaaggagatccccgacac

SEQ ID NO: 744 - DNA, 361 bp, unidentified
  misc_feature 1..361: note = Description of Unknown: RMRPCCDC107 bidirectional promotoer sequence
  source 1..361: mol_type = other DNA
tgccggcccacgggtggagggatcgggcgggcggtgccgaagcggtccggcattggccggccgccccaacgcgcacgcgcacgcgagcaggccggccggctccggggaggccacgcccactccccgtagggcggggccagaccatatttgcataagatagtgtcattctagctttcctgtatttgttcatttcgtgtctattagctattctgctagccacaatgcctctgaaagcctatagtcttagaaagttatgcccgaaaacggtttttttaatctcacgccaccaactttctcaccctaatcataaaacacaatttctttagggctataaaatactactctgtgaagctgaggacgt

SEQ ID NO: 745 - DNA, 376 bp, unidentified
  misc_feature 1..376: note = Description of Unknown: tRNA(Lys)ALOXE3 bidirectional promotoer sequence
  source 1..376: mol_type = other DNA
tctttccgctccaggaccgccctgggcctgcaggatcctgggcgggagcccaggtgtccgggatctgggccactagggactggggaggaacctctcagagaagcccatagcccgcagcggccccgcgcggccggttccggcgccgcactgttccagcctctactatggtacagtccctgcgtcgcagcctcggcgggggctctaagaacgggaggcagaaaaagctcaatcagcagcaggcgagcttcacccgctgcttccaaatctgtgccaaaatattctatgctgcacagataaaatcctctgtcggttctacaagcctggcttttcctatagagaaccctcttataagcaaaaagtaaagctctcgtgaaga

SEQ ID NO: 746 - DNA, 412 bp, unidentified
  misc_feature 1..412: note = Description of Unknown: RNU69MED16 bidirectional promotoer sequence
  source 1..412: mol_type = other DNA
gagggcagtcaccagctcctggcccgtgcgccaagctcagcgggcgtccgcggtgcgatcttccctagcgcctcgggtctggcgccgccatcttcctcggtaacaaccagtcgcctgaggcgtggggccgcctcccaaagacttctgggagggcggtgcggctcaggctctgccccgcctccggggctatttgcatacgaccatttccagtaattcccagcagccaccgtagctatatttggtagaacaacgagcactttctcaactccagtcaataactacgttagttgcattacacattgggctaatataaatagaggttaaatctctaggtcatttaagagaagtcggcctatgtgtacagacatttgttccaggggctttaaatagctggtggtggaactcaatattc

SEQ ID NO: 747 - DNA, 484 bp, unidentified
  misc_feature 1..484: note = Description of Unknown: tRNA (Gly)DPP9 bidirectional promotoer sequence
  source 1..484: mol_type = other DNA
taaccgctcagctgacctcaggagggcaggggtgccttctaaagggtccagagagcctccattccagctgcaggcgtgggacacagaccgggacgtggggcggcggccggactgggcaggtcgtcccgggtccagcggcgcctcacggtcgcggctccatgcccgggactgcgaccccggaagtggcgggagcgggggacgacagccgcggcggacacaggggacccgccggctcaggcacctttgacccggaagttgagcgacccaggcggcggcctgggattggacaccaccaggcacgtaccaaggcgtccgcggcgcttgggggggagcccgcggcgcggcggcctaaggtgcgtaacgccccatgaacgacatcttccggtgggttagggagagacacccccctgtgacttggtatcactcagtcaaacccatgatcccccactattaaggatatccggagaggatgctacctatcagg

SEQ ID NO: 748 - DNA, 861 bp, unidentified
  misc_feature 1..861: note = Description of Unknown: SNORD13 C8orf41 bidirectional promotoer sequence
  source 1..861: mol_type = other DNA
tcctgactgcagcaccagaaggctggtctctcccacagaacgaggatggaggcggggagggatccgttgaagagggaaggagcgatcacccaaagagaactaaaatcaaataaaataaaacagagagatgtcttggaggagggggcgagtctgaccgggataagaataaagagaaagggtgaacccgggaggcggagtttgcagtgagccgagatcgcgccactgcactccagcctgggcgacagagtgagactccgtctcagtaaaaaaaaaaaaaaaaaaaagaataaagaggaaaggacgcaagaaagggaaaggggactctcagggagtaaaagagtcttacacttttaacagtgacgttaaaagactactgttgcctttctgaagactaaaaagaaaaaaaacttaaaaatttaaagaaataaacttctgagccatgtcaccaacttaaccacccccaggtacctgcaacggctcgcgcccgccggtgtctaacaggatccggacctagctcatattgctgccgcaaaacgcaaggctagcttccgccagtactgccgcaacaccttcttatttcacgacgtatggtcgtaaagcaataaagatccaggctcgggaaaatgacggagaggtggaactatagagaataaatttgcatatataataatccgctcgctaattgtgtttctgttttcctttgctaaggtagaaacaaaagaataatcacagaatctcagtgggactttgaaaatatccaggattttatacgtgaagaatggatgtatcgcattacggtagtcaccctatgtgtaaattagtggcacatacttggcactccttaatgtcaactataagatg

SEQ ID NO: 749 - DNA, 525 bp, unidentified
  misc_feature 1..525: note = Description of Unknown: RNU62THEM259 bidirectional promotoer sequence
  source 1..525: mol_type = other DNA
gcctcccagcgtcgcgccctaacgacccgcaagtgtccgagggcgcctcccggccgccatcggccgccctcgcagccgccgctctcctcacggcctcccggccgccgccgccatcttccgctttctcgtccggctgcggcgctgctgacgctagcgagtcgccacgccgggcaagagcggcccccctgcgcccgcagagaacgctgggatgccagcggcgcccgcggaggcctcaccccctacctcggccgctccagggggcgggcctgcatctgggccacctcttttgcatattggcacccacaatccaccgcggctatgaggccagtataaggcggtaaaattacgataagatatgggattttacgtgatcgaagacatcaaagtaagcgtaagcacgaaagttgttctgcaacataccactgtaggaaattatgctaaatatgaaaccgaccataagttatcctaaccaaaagatgatttgattgaagggcttaaaataggtgtgacagtaacccttgagtc

SEQ ID NO: 750 - DNA, 172 bp, Mus musculus
  source 1..172: mol_type = other DNA
cgctcttgaaggacgacgtcatcatcccttgcccggatgcgcgggcttcttgtctagcacaggagcctggggtagagcgcatgcaaattacgcgctgtgctttgtgggaaatcaccctaaacgaaaaatttattcctctttcgagccttatagtggcggccggtctacatcc

SEQ ID NO: 751 - DNA, 201 bp, Rattus norvegicus
  source 1..201: mol_type = other DNA
ggctgatgagcttcccccgcccactaggagtgtgaagacctgccgccataataagactccaaaagacagtgaatttaacacttacggtgacttcccacaaagcacagcgtgtaatttgcatgcgctctagcccaggctccagctccggaccagaagcccgcgcatcccggcaaagggtgatgacgtcgtccttcaagcgct

SEQ ID NO: 752 - DNA, 176 bp, Mus musculus
  source 1..176: mol_type = other DNA
ttcaggatgtagaccggccgccactataaggctcgaaagaggaataaatttttcgtttagggtgatttcccacaaagcacagcgcgtaatttgcatgcgctctaccccaggctcctgtgctagacaagaagcccgcgcatccgggcaagggatgatgacgtcgtccttcaagagcg

SEQ ID NO: 753 - DNA, 176 bp, Rattus norvegicus
  source 1..176: mol_type = other DNA
aggagtgtgaagacctgccgccataataagactccaaaagacagtgaatttaacacttacggtgacttcccacaaagcacagcgtgtaatttgcatgcgctctagcccaggctccagctccggaccagaagcccgcgcatcccggcaaagggtgatgacgtcgtccttcaagcgct

SEQ ID NO: 754 - DNA, 225 bp, Dipodomys ordii
  source 1..225: mol_type = other DNA
aggaaagacttcgctgaggcagactttataaggctcccgcgcagaaagaaactttatagttatggtgatttcccacaagccactgcgtcatgcaaataaagcagggtacggcttccatgtaccttaaggtttttttctaggccgcgtacgctctgcgtattcagccacgtgaccctgagccagtggttgttgggagcacgttgtggacctctgcgtttggattcc

SEQ ID NO: 755 - DNA, 222 bp, Ictidomys tridecemlineatus
  source 1..222: mol_type = other DNA
gaaagggactccgcacaagcagagtttataaggctcccatctgtacagccatttctcggtcatggtaactacccacaacacacagcgatatgcaaatatagcagagcgtgtcttcccgcgcgcgcctggtcgtctcggcgccggcgcgctgcgtggggcggaactgtgacagagaccctgcgattcctgggagctggctgatgacatcagtgtctaacctcc

SEQ ID NO: 756 - DNA, 225 bp, Cavia porcellus
  source 1..225: mol_type = other DNA
gagaaagaaaggctcaaacctagccttataaggctcccaaatgtcggtatattttttggttatggtgacttcccacaatgcatagcgatatgtagatattgccaggagtacctcccacttctggtcctgtcagctcttttctaggacgcgcgcgctgcaggtttccagcctgtgattgggccagcaattccgggaatgaattgatgacgtcagcgtttgaattcc

SEQ ID NO: 757 - DNA, 211 bp, Ochotona princeps
  source 1..211: mol_type = other DNA
gggggaagctgggctcgatcagcctttataaagctccaaaaactcaagacatttttctgttacggtggcttcccacagtacacagcgacatgcaaatagcttgccaatgaattcgcggaccgcttcccgccccggcgcaggcgcgcggacgctgtctcccctggacgcgcgctcgcggttcccgggagctggctgatgacgttcggtctcc

SEQ ID NO: 758 - DNA, 211 bp, Oryctolagus cuniculus
  source 1..211: mol_type = other DNA
ggggagaggtggatccgaacagactttataaagctccgaaagcccaaggcatctttcccttacggtagcttcccacaagacatagcgacatgcaaatttcttgaagtatgcttcagacgcgcttctcgccacagcgcaagcgcgctgtgtgctgacgcgggaacgggccagggcgcggttcccgggagcgggttgatgacgttagatctcc

SEQ ID NO: 759 - DNA, 229 bp, Callithrix jacchus
  source 1..229: mol_type = other DNA
gaggaaaagtagtcccacagacaacttataagattcccataccctaagacatttcacgattatggtgacttcccagaagacacagcgacatgcaaatattgcaggtcgtgtttcgcctgtccctcacagtcgtcttcctgccagggcgcacgcgcgctgggtttcccgccaactgacgctgggctcgcgattccttggagcgggttgatgacgtcagcgtttgaattcc

SEQ ID NO: 760 - DNA, 230 bp, Chlorocebus sabaeus
  source 1..230: mol_type = other DNA
ggggaagggtggtcccttacagaacttataagattcccaaactcaaagacatttcacgtttatggtgacttcccagaagacatagcgacatgcaaatattgcagggcgtcacacccctctccctcacagtcatcttcctgccagggcgcacgcgcgctgggtgttctcgcgtagtgacactgggcccgcgattccttggagcgggttgatgacgtcagcgttcgaattcc

SEQ ID NO: 761 - DNA, 230 bp, Macaca mulatta
  source 1..230: mol_type = other DNA
ggggaagggtggtcccacacagaacttataagattcccatactcaaagacatttctcgtttatggtgacttcccagaagacacagcgacatgcaaatattgtagggcgtcacacccctgtccctcacagtcatcttcctgccagggcgcacgcgcgctgggtgttcccgcgtagtgacactgggcccgcgattccttggagcgggttgatgacgtcagcgttcgaattcc

SEQ ID NO: 762 - DNA, 231 bp, Papio anubis
  source 1..231: mol_type = other DNA
ggggaaaggtggtaccatacagaacttataagattcccatactcaaagacatttcacgattatggtgacttcccagaagacacagcgacatgcaaatattgtagggcgtcacaccccctgtccctcacagtcatcttcctgccagggcgcacgcgcgctgggtgttcccgcgtagtgacactgggcccgcgattccttggagcgggttgatgacgtcagcgttcgaattcc

SEQ ID NO: 763 - DNA, 230 bp, Gorilla gorilla
  source 1..230: mol_type = other DNA
gggaaagggtggtcccacacagaacttataagactcccatatccaaagacatttcacggttatggtgatttcccagaacacatagcgacatgtaaatattgcagggcgccactccccagtccctcacagccatcttcctgccagggcgcacgcgcgctgggtgttcccgcctagtgacactgggcccgcgattccttggagcgggttgatgacgtcagcgttcgaattcc

SEQ ID NO: 764 - DNA, 230 bp, Homo sapiens
  source 1..230: mol_type = other DNA
gggaaaaagtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcgaattcc

SEQ ID NO: 765 - DNA, 230 bp, Pan troglodytes
  source 1..230: mol_type = other DNA
gggaaagggtggtgccacacagaacttataagattcccatatgcaaagacatttcacgtttatggtgatttcccagaacacatagcgacatgcaaatattgcagggcgccactcccctgtccctcactgccatcttcctgccagggcgcacgcgcgctgggtgttcccgcctagtgacactgggcccgcgattccttggagcgggttgatgacgtcagcgttcgaattcc

SEQ ID NO: 766 - DNA, 229 bp, Pongo abelii
  source 1..229: mol_type = other DNA
gagaaagggtggtcccgtccagaacttataagattcccatacccaaagacatttcacgtttatggtgacttcccagaatgcatagcgacatgcaaatattgcagggcgtcactcccctgtccctcacagccatcttcctgccagggcgcccgcgcgctggtgttcccgcctagtgacactgggcccacgattccttggagcgggttgatgacgtcagcgctcgtattcc

SEQ ID NO: 767 - DNA, 223 bp, Nomascus leucogenys
  source 1..223: mol_type = other DNA
ggggaaaagtagtagaccttataagattcccaaacccaaagacatttctcgtttatggtgacttcccagaagacatagcgacatgcaaatattgcagggcgccactcccctgtccctcacagccatcttcctgccagggcgcacgcgcgctgggtgttcccgcctagtgacactcggcccgcgattccttggagcgggttgatgacgtcagcgttcgaattcc

SEQ ID NO: 768 - DNA, 227 bp, Tarsius sp.
  source 1..227: mol_type = other DNA; note = Tarsius syrichta
gcgagagggtgggtccacacagagcttataaggttcacaagtaaagatatttcacggtgacggtgacttcccacaatacactgcgacatgcaaatatagccgggcgtgcctccccgatcccggaagagcgactcctagccagtgcgcacgcgcgctgcgtgttcgcgtcctaggtcgctgggcccgcggttcctgggagcgggtggtgacgtcagcggcccagcttc

SEQ ID NO: 769 - DNA, 230 bp, Otolemur garnettii
  source 1..230: mol_type = other DNA
gcctaaaagggcgcttgcacagaatttataaggttcccaaacagagacacatttcattattatggtgacttcccacaatgcacagcgccatgcaaatatgctaggacgcctccccccgctaccttaaggtcgtcaactaaccagtgcgcgcgcgcactgcgcgtttcccgccggtgactcaatgcccgcgtttggtgggagctagttggtgacctcagttctggaggctc

SEQ ID NO: 770 - DNA, 226 bp, Ailuropoda melanoleuca
  source 1..226: mol_type = other DNA
agggaaagccgcgcctggggcggatttataaggcttccatatctaaaggcatttcacagtcatggtgacttcccacaatacatagcaacatgcaaatatcgcggggagaacctcccctgtcccttgtacgcggcttctaaagacgcacgcgcgctctgtgttcccgccctgtgactctaggcgggcaattcctgggacagtgttctgacgggaacgttcaggctcc

SEQ ID NO: 771 - DNA, 227 bp, Mustela putorius
  source 1..227: mol_type = other DNA
gggaaagggtggacccaccgagcatttataaggctcccgcatctaaagacattttacagttatggtgacttcccacaacgcgtagcaacatgcaaatatcgtggagagtaccgcccctgtcccatgcacgcgtcttctcagcagcacgcacgcgcgctgtgttcccgccctgtgactccaggcgggtatttccaggggcgggttgctgacaggaacgttcaggcttc

SEQ ID NO: 772 - DNA, 229 bp, Canis sp.
  source 1..229: mol_type = other DNA; note = Canis familiaris
gcagcgcagccctctcgccgcttataaagtgccgcccgcacggcccttctcgctcacggcgacttcccataacacacagcagcatgcaaataccgcggggagccccgccccgccccggcccccgcaccgcctcgggacgcatgcgccggctctccgttcccgccttgggccggcggcgggcgggcgggcgagcgggcgggagcggctccggcggggacgagcgggcgcc

SEQ ID NO: 773 - DNA, 228 bp, Felis catus
  source 1..228: mol_type = other DNA
gggaaagggtggccccgccgagcatttataagactcccatacctaaagacatttctcagttatggtgatttcccacaacacacagcaacatgcaaatatcgaggggtgtaccgcccctgtcctttgtagacgtcttctctccaggacgcacgcgcgctgtattcccgccttgtgactctaggcgggcgattcctgggagagggttgatgacgtccaagttctggcttc

SEQ ID NO: 774 - DNA, 227 bp, Equus caballus
  source 1..227: mol_type = other DNA
gggggaaaacagcccatggctgcatttataagactcacagatctaaagccatttcacgaatagggtgacttcccacaatacacagcgacatgcaaacatagcggggcgtgcctttcctgtaccctgtgggcatctctcctggacgcacgcgcgccgggtgttcccgcgctgtgactctaggcaagcgcttcctgggagagagttgatgacggcagcattcgggctcc

SEQ ID NO: 775 - DNA, 225 bp, Myotis lucifugus
  source 1..225: mol_type = other DNA
gggagaaggaggcgtagaggatatataaggcccccttatgtgtagtccttttacggttagggtgacttcccacaacgcatagcgacatgcaaatttgacgggcgtgcctcctctgtccctgcgggcaacttctctcctggacgcgcgcgcgctgcgtgttcccgccttttgactccagccgagcgaatcctgggagagggcaggtgacgtcaacagtcaggctcg

SEQ ID NO: 776 - DNA, 227 bp, Pteropus vampyrus
  source 1..227: mol_type = other DNA
gcgagaaaaattcttcacgcagaatatataaggatcccatatctgaagacattttacgattacggcgatttcccacaacacatagcgacatgtaaatgtagtggggcatgcctcccctgtcccttgtgggcagcttctcgccagaacgcacgcgcggtgcgtgttcccgccttgtgactaagttggcgagtcagggaggagattgatgacgtcagctcacccgctcc

SEQ ID NO: 777 - DNA, 218 bp, Bos taurus
  source 1..218: mol_type = other DNA
ggcaaacaccgcacgcaaatagcacttataatgtgctcatacctagagccacttttcggttacggtgacttctcaaaaagacagtggaacatgcaaatattacagtgcgtcccgcccctggtaggtctacgctaggacgcacgcgcactacggttcccgcctatagactgcgctggcgattcctgggagcggactgatgacgtcagcgttcgggatcc

SEQ ID NO: 778 - DNA, 216 bp, Ovis aries
  source 1..216: mol_type = other DNA
ggcgaacaatgcgcgcaaacagcatttataatgagctcatacctaaagccactttacggttacggtgacttcccacaagacattgcggcatgcaaatattttagtgcgtcccgcccctggtagttccacgctaggacgcacacgcactacggttcccgcctttagactgcgctggcgattccaggagcggactgatgacgtcagcgttggggctcc

SEQ ID NO: 779 - DNA, 218 bp, Tursiops truncatus
  source 1..218: mol_type = other DNA
gccgaaaaccaggctcaagccacatttataaggctcccaaatctaagtacatttgtcggttatggtgacttcccgcaccacattgcgacatgcaaatactgcggagcgtccctcccctggcaactcctcgctgggacgcacgcgcgctacgtgctcccgccttttgactgcgccggcgatacttgggagagggttgatgacgtcagcgttctggctcc

SEQ ID NO: 780 - DNA, 226 bp, Vicugna pacos
  source 1..226: mol_type = other DNA
gggaaagggtgggctcacgcagcctttataagactcccaaacttaaagacatttctcggttatggcgacttcccacaagacatagcgacatgcaaatactgcagggcgccgacccggtcctgtgcagccatctttcggctgggacgcacgcgcgctgcgtgttcccgccctgtgactgcgccggcgattactgggagaggattgatgacgtcaacgttcgggttcc

SEQ ID NO: 781 - DNA, 228 bp, Sus scrofa
  source 1..228: mol_type = other DNA
gtaggaaaactgcttctgtgagcacttataaaactcccataagtagagagatttcatagttatggtgatttcccataagacattgcgacatgcaaatattgtggcgcgttcgtccccgtccggtgcaggcagcttcgctccaggacgcacgcgcaatacatgttcccgccttgagactgcgccggcagattcctaggaagtggttgatgacgtcgatgttagggatcc

SEQ ID NO: 782 - DNA, 210 bp, Erinaceus europaeus
  source 1..210: mol_type = other DNA
gcctaaaccggctctttcgacagacttataaggacctcttatcttaggacatttttttgttagggtaacttcccacgatgcatagcgatatgtaaatatggcgccgcgagtctctcctaggcgtctccccaggacgcaggcgcactgcttgttcccgcgttaacattgctgattctgggagactgctgatgacgtcagcgtccagtctac

SEQ ID NO: 783 - DNA, 211 bp, Choloepus hoffmanni
  source 1..211: mol_type = other DNA
agaaaaaaatagtttatgctggatttataagattcccaaatctaaagccatttcacagttacggtgattccccactacacacggcgatatgcaaatatagcggaagtgttcctgaggcgtggtaaagcgcgcgcgcgctgagagttcccgccctgtggtgctgggctggagatgcctgagaactggctgatgacggcaacgttcgggctcc

SEQ ID NO: 784 - DNA, 208 bp, Dasypus novemcinctus
  source 1..208: mol_type = other DNA
aaagcgatagttttttaaactggacttataaggcacccatatctacgtatatttcatggttagggtgatttcccacaacacatagcgaaatgcaaatattggagggcgctgaggcgtggtcgggcgcaagcgcgctgcgacttcccgcctttcggccctaggccccagattcctgggagctggatgatgacgttgacgttcggatacc

SEQ ID NO: 785 - DNA, 227 bp, Loxodonta africana
  source 1..227: mol_type = other DNA
gggaaggaacaaattcgtcaggatttataagactctcagagctgtagacatttcacagttagggcgatgtcccacaatacatagcaacatgcaaatattctaggaggccagcctccccgtccgcgtggtcatcttctcgctagggcgcacgcccgctgcgtgttcccgctctgtgaccaggcaggcgattcctgagaaccgcttggtgacgtcagtgttctggctcc

SEQ ID NO: 786 - DNA, 225 bp, Procavia capensis
  source 1..225: mol_type = other DNA
agggtaaatcggcgctgctcagcatttaaaagaatcccaaatgtgtcgccattttacgcttagggtgatatcccacaagacacagcgacatgcaaatatcgtgagtctctgtttccctgtccacgagggcgtcctctcgctggggcgcacgcgcggtgtgtgtgcccccgttgtgtgttcccgcgattccaaagaactggttgataacgttagacttccggctgc

SEQ ID NO: 787 - DNA, 230 bp, unidentified; identical to SEQ ID NO: 739
  misc_feature 1..230: note = Description of Unknown: H1 WT promoter sequence
  source 1..230: mol_type = other DNA

SEQ ID NO: 788 - DNA, 23 bp, Homo sapiens
  source 1..23: mol_type = other DNA
cggccaggctctcacctcggggg

SEQ ID NO: 789 - DNA, 23 bp, Homo sapiens
  source 1..23: mol_type = other DNA
ctctcacctcgggggctccgcgg

SEQ ID NO: 790 - DNA, 23 bp, Homo sapiens
  source 1..23: mol_type = other DNA
gctgccctccaccggacccgggg

SEQ ID NO: 791 - DNA, 23 bp, Homo sapiens
  source 1..23: mol_type = other DNA
cagcgccgccttttgtgctgcgg

SEQ ID NO: 792 - DNA, 23 bp, Homo sapiens
  source 1..23: mol_type = other DNA
agcctggccgagttgtggggggg

SEQ ID NO: 793 - DNA, 23 bp, Homo sapiens
  source 1..23: mol_type = other DNA
gagcctggccgagttgtgggggg

SEQ ID NO: 794 - DNA, 23 bp, Homo sapiens
  source 1..23: mol_type = other DNA
catcgcctgtgacagcgccccgg

SEQ ID NO: 795 - DNA, 23 bp, Homo sapiens
  source 1..23: mol_type = other DNA
tcgtgcggtgtccggatgcacgg

SEQ ID NO: 796 - DNA, 23 bp, Homo sapiens
  source 1..23: mol_type = other DNA
cgcggccgcagcacaaaaggcgg

SEQ ID NO: 797 - DNA, 23 bp, Homo sapiens
  source 1..23: mol_type = other DNA
ctgcggccgcggagcccccgagg

SEQ ID NO: 798 - DNA, 23 bp, Homo sapiens
  source 1..23: mol_type = other DNA
gcagggacggggctaggggtcgg

SEQ ID NO: 799 - DNA, 23 bp, Homo sapiens
  source 1..23: mol_type = other DNA
gcgctgctgcagctccgctccgg

SEQ ID NO: 800 - skipped

SEQ ID NO: 801 - DNA, 23 bp, Homo sapiens
  source 1..23: mol_type = other DNA
cgccttttgtgctgcggccgcgg

SEQ ID NO: 802 - DNA, 23 bp, Homo sapiens
  source 1..23: mol_type = other DNA
agggtgttcgggtctcatggagg

SEQ ID NO: 803 - DNA, 23 bp, Homo sapiens
  source 1..23: mol_type = other DNA
tctcgaagtacacactgggtagg

SEQ ID NO: 804 - DNA, 23 bp, Homo sapiens
  source 1..23: mol_type = other DNA
accatcataccaggggccagagg

SEQ ID NO: 805 - DNA, 23 bp, Homo sapiens
  source 1..23: mol_type = other DNA
actgttggcaaaaggctcagggg

SEQ ID NO: 806 - DNA, 23 bp, Homo sapiens
  source 1..23: mol_type = other DNA
ctacatgagcaggatgcaggggg

SEQ ID NO: 807 - DNA, 23 bp, Homo sapiens
  source 1..23: mol_type = other DNA
ctttgtgtctaccctaagtgagg

SEQ ID NO: 808 - DNA, 23 bp, Homo sapiens
  source 1..23: mol_type = other DNA
ccacatctcgaagtacacactgg

SEQ ID NO: 809 - DNA, 23 bp, Homo sapiens
  source 1..23: mol_type = other DNA
cacatctcgaagtacacactggg

SEQ ID NO: 810 - DNA, 23 bp, Homo sapiens
  source 1..23: mol_type = other DNA
tgccaatcatggtccagacaggg

SEQ ID NO: 811 - DNA, 23 bp, Homo sapiens
  source 1..23: mol_type = other DNA
cttcgagatgtggtaccccttgg

SEQ ID NO: 812 - DNA, 23 bp, Homo sapiens
  source 1..23: mol_type = other DNA
atccagagttcgagctgacgagg

SEQ ID NO: 813 - DNA, 23 bp, Homo sapiens
  source 1..23: mol_type = other DNA
ctgttggcaaaaggctcaggggg

SEQ ID NO: 814 - DNA, 23 bp, Homo sapiens
  source 1..23: mol_type = other DNA
tcatggtccagacagggcgcagg

SEQ ID NO: 815 - DNA, 23 bp, Homo sapiens
  source 1..23: mol_type = other DNA
cccccaccctgcccaccaagggg

SEQ ID NO: 816 - DNA, 23 bp, Homo sapiens
  source 1..23: mol_type = other DNA
gagtgcagtcagaagtgtctggg

SEQ ID NO: 817 - DNA, 23 bp, Homo sapiens
  source 1..23: mol_type = other DNA
gtctgggtccagcttgcgcatgg

SEQ ID NO: 818 - DNA, 23 bp, Homo sapiens
  source 1..23: mol_type = other DNA
gtaggcgtcaggtccttctcggg

SEQ ID NO: 819 - DNA, 23 bp, Homo sapiens
  source 1..23: mol_type = other DNA
gctacatgagcaggatgcagggg

SEQ ID NO: 820 - DNA, 23 bp, Homo sapiens
  source 1..23: mol_type = other DNA
ctgctgctgcttgtgctcagtgg

SEQ ID NO: 821 - DNA, 23 bp, Homo sapiens
  source 1..23: mol_type = other DNA
cctggtatgatggtgaacactgg

SEQ ID NO: 822 - DNA, 23 bp, Homo sapiens
  source 1..23: mol_type = other DNA
gggcccagcccctccccaggtgg

SEQ ID NO: 823 - DNA, 23 bp, Homo sapiens
  source 1..23: mol_type = other DNA
acaccctctccttcctttggggg

SEQ ID NO: 824 - DNA, 23 bp, Homo sapiens
  source 1..23: mol_type = other DNA
agctacatgagcaggatgcaggg

SEQ ID NO: 825 - DNA, 23 bp, Homo sapiens
  source 1..23: mol_type = other DNA
ttggcaaaaggctcagggggcgg

SEQ ID NO: 826 - DNA, 23 bp, Homo sapiens
  source 1..23: mol_type = other DNA
acaagcagcagcaggaaggtagg

SEQ ID NO: 827 - DNA, 23 bp, Homo sapiens
  source 1..23: mol_type = other DNA
ttgccaatcatggtccagacagg

SEQ ID NO: 828 - DNA, 23 bp, Homo sapiens
  source 1..23: mol_type = other DNA
gcgcatggatgcctcacttaggg

SEQ ID NO: 829 - DNA, 23 bp, Homo sapiens
  source 1..23: mol_type = other DNA
ctgcagtgccagagtggcagtgg

SEQ ID NO: 830 - DNA, 23 bp, Homo sapiens
  source 1..23: mol_type = other DNA
tcccccaccctgcccaccaaggg

SEQ ID NO: 831 - DNA, 23 bp, Homo sapiens
  source 1..23: mol_type = other DNA
agtggcagtggcttccttgaggg

SEQ ID NO: 832 - DNA, 23 bp, Homo sapiens
  source 1..23: mol_type = other DNA
cacctcgtcagctcgaactctgg

SEQ ID NO: 833 - DNA, 23 bp, Homo sapiens
  source 1..23: mol_type = other DNA
gcggctctccacctggggagggg

SEQ ID NO: 834 - DNA, 23 bp, Homo sapiens
  source 1..23: mol_type = other DNA
ggtccaggatttcctcaaagggg

SEQ ID NO: 835 - DNA, 23 bp, Homo sapiens
  source 1..23: mol_type = other DNA
tcactttcaagtgaggtccaggg

SEQ ID NO: 836 - DNA, 23 bp, Homo sapiens
  source 1..23: mol_type = other DNA
ggaaatcctggacctgcagtggg

SEQ ID NO: 837 - DNA, 23 bp, Homo sapiens
  source 1..23: mol_type = other DNA
tcacagccacctcctccccgtgg

SEQ ID NO: 838 - DNA, 23 bp, Homo sapiens
  source 1..23: mol_type = other DNA
aacatcatcactttcaagtgagg

SEQ ID NO: 839 - DNA, 23 bp, Homo sapiens
  source 1..23: mol_type = other DNA
cctcaaaggggacctcccaaagg

SEQ ID NO: 840 - DNA, 23 bp, Homo sapiens
  source 1..23: mol_type = other DNA
cttcctggggcgcttccacgggg

SEQ ID NO: 841 - DNA, 23 bp, Homo sapiens
  source 1..23: mol_type = other DNA
cctggggcgcttccacggggagg

SEQ ID NO: 842 - DNA, 23 bp, Homo sapiens
  source 1..23: mol_type = other DNA
ctttcgcaagtgcttgatgtcgg

SEQ ID NO: 843 - DNA, 23 bp, Homo sapiens
  source 1..23: mol_type = other DNA
aggaaatcctggacctgcagtgg

SEQ ID NO: 844 - DNA, 23 bp, Homo sapiens
  source 1..23: mol_type = other DNA
tcaagtgaggtccagggcagggg

SEQ ID NO: 845 - DNA, 23 bp, Homo sapiens
  source 1..23: mol_type = other DNA
gcttgatgtcggtttctttgagg

SEQ ID NO: 846 - DNA, 23 bp, Homo sapiens
  source 1..23: mol_type = other DNA
gcgcttccacggggaggaggtgg

SEQ ID NO: 847 - DNA, 23 bp, Homo sapiens
  source 1..23: mol_type = other DNA
tgggtgacttgagatccctgtgg

SEQ ID NO: 848 - DNA, 23 bp, Homo sapiens
  source 1..23: mol_type = other DNA
cctgggcgcagaactccatgagg

SEQ ID NO: 849 - DNA, 23 bp, Homo sapiens
  source 1..23: mol_type = other DNA
cctgcacaagattatccacaggg

SEQ ID NO: 850 - DNA, 23 bp, Homo sapiens
  source 1..23: mol_type = other DNA
gtaaggagggggtgacagggcgg

SEQ ID NO: 851 - DNA, 23 bp, Homo sapiens
  source 1..23: mol_type = other DNA
accagtcaaccagtaaggagggg

SEQ ID NO: 852 - DNA, 23 bp, Homo sapiens
  source 1..23: mol_type = other DNA
ccagtcaaccagtaaggaggggg

SEQ ID NO: 853 - DNA, 23 bp, Homo sapiens
  source 1..23: mol_type = other DNA
ggaccagtcaaccagtaaggagg

SEQ ID NO: 854 - DNA, 23 bp, Homo sapiens
  source 1..23: mol_type = other DNA
tccatgaggatgcagtagcaggg

SEQ ID NO: 855 - DNA, 23 bp, Homo sapiens
  source 1..23: mol_type = other DNA
acctgcacaagattatccacagg

SEQ ID NO: 856 - DNA, 23 bp, Homo sapiens
  source 1..23: mol_type = other DNA
gaccagtcaaccagtaaggaggg

SEQ ID NO: 857 - DNA, 23 bp, Homo sapiens
  source 1..23: mol_type = other DNA
ggataatcttgtgcaggtgcagg

SEQ ID NO: 858 - DNA, 23 bp, Homo sapiens
  source 1..23: mol_type = other DNA
tccctgctactgcatcctcatgg

SEQ ID NO: 859 - DNA, 23 bp, Homo sapiens
  source 1..23: mol_type = other DNA
gccagctgtatgaggtactgcgg

SEQ ID NO: 860 - DNA, 23 bp, Homo sapiens
  source 1..23: mol_type = other DNA
cccaagtgagtaagcaagcaggg

SEQ ID NO: 861 - DNA, 23 bp, Homo sapiens
  source 1..23: mol_type = other DNA
ccagctgtatgaggtactgcggg

SEQ ID NO: 862 - DNA, 23 bp, Homo sapiens
  source 1..23: mol_type = other DNA
gatgcagtagcagggagcctggg

SEQ ID NO: 863 - DNA, 23 bp, Homo sapiens
  source 1..23: mol_type = other DNA
tacctcatacagctggccctggg

SEQ ID NO: 864 - DNA, 23 bp, Homo sapiens
  source 1..23: mol_type = other DNA
ttactggttgactggtccatggg

SEQ ID NO: 865 - DNA, 23 bp, Homo sapiens
  source 1..23: mol_type = other DNA
gtacctcatacagctggccctgg

SEQ ID NO: 866 - DNA, 23 bp, Homo sapiens
  source 1..23: mol_type = other DNA
gcctgggtgcacacacccctggg

SEQ ID NO: 867 - DNA, 23 bp, Homo sapiens
  source 1..23: mol_type = other DNA
ttcattgcggatcacctcagggg

SEQ ID NO: 868 - DNA, 23 bp, Homo sapiens
  source 1..23: mol_type = other DNA
tctcagacacaggttcattgcgg

SEQ ID NO: 869 - DNA, 23 bp, Homo sapiens
  source 1..23: mol_type = other DNA
agtagcctggatggcccctgagg

SEQ ID NO: 870 - DNA, 23 bp, Homo sapiens
  source 1..23: mol_type = other DNA
gttcattgcggatcacctcaggg

SEQ ID NO: 871 - DNA, 23 bp, Homo sapiens
  source 1..23: mol_type = other DNA
atgtcgaccttctcagacacagg

SEQ ID NO: 872 - DNA, 23 bp, Homo sapiens
  source 1..23: mol_type = other DNA
gtcgtaggtgattagcatgctgg

SEQ ID NO: 873 - DNA, 23 bp, Homo sapiens
  source 1..23: mol_type = other DNA
tgtctgagaaggtcgacatctgg

SEQ ID NO: 874 - DNA, 23 bp, Homo sapiens
  source 1..23: mol_type = other DNA
cctttgcagggacagtagcctgg

SEQ ID NO: 875 - DNA, 23 bp, Homo sapiens
  source 1..23: mol_type = other DNA
gctcttgtcactcagctccttgg

SEQ ID NO: 876 - DNA, 23 bp, Homo sapiens
  source 1..23: mol_type = other DNA
aggaatctacgtctttgtagggg

SEQ ID NO: 877 - DNA, 23 bp, Homo sapiens
  source 1..23: mol_type = other DNA
gcaccacgccaaaggacctaggg

SEQ ID NO: 878 - DNA, 23 bp, Homo sapiens
  source 1..23: mol_type = other DNA
gaaaccatctgggcaactggagg

SEQ ID NO: 879 - DNA, 23 bp, Homo sapiens
  source 1..23: mol_type = other DNA
tctgggcaactggagggcacggg

SEQ ID NO: 880 - DNA, 23 bp, Homo sapiens
  source 1..23: mol_type = other DNA
tgtggagagtacatcagctgagg

SEQ ID NO: 881 - DNA, 23 bp, Homo sapiens
  source 1..23: mol_type = other DNA
gacttaaagtaagtctcctgggg

SEQ ID NO: 882 - DNA, 23 bp, Homo sapiens
  source 1..23: mol_type = other DNA
ccagatgcagcaggatctgtcgg

SEQ ID NO: 883 - DNA, 23 bp, Homo sapiens
  source 1..23: mol_type = other DNA
aggcaatgtccagatgcagcagg

SEQ ID NO: 884 - DNA, 23 bp, Homo sapiens
  source 1..23: mol_type = other DNA
ttaagtcccaggtgtgctgtggg

SEQ ID NO: 885 - DNA, 23 bp, Homo sapiens
  source 1..23: mol_type = other DNA
tgatgtactctccacaccccagg

SEQ ID NO: 886 - DNA, 23 bp, Homo sapiens
  source 1..23: mol_type = other DNA
gcaggatctgtcggaatgatggg

SEQ ID NO: 887 - DNA, 23 bp, Homo sapiens
  source 1..23: mol_type = other DNA
tcaccagttcctcttctaggcgg

SEQ ID NO: 888 - DNA, 23 bp, Homo sapiens
  source 1..23: mol_type = other DNA
ctgtctgcaccgcctagaagagg

SEQ ID NO: 889 - DNA, 23 bp, Homo sapiens
  source 1..23: mol_type = other DNA
ttgaaaagattaagtcagaaggg

SEQ ID NO: 890 - DNA, 23 bp, Homo sapiens
  source 1..23: mol_type = other DNA
gtgagttcccgtgcttggggagg

SEQ ID NO: 891 - DNA, 23 bp, Homo sapiens
  source 1..23: mol_type = other DNA
gcaccgcctagaagaggaactgg

SEQ ID NO: 892 - DNA, 23 bp, Homo sapiens
  source 1..23: mol_type = other DNA
gagttccagctgcaacatgaggg

SEQ ID NO: 893 - DNA, 23 bp, Homo sapiens
  source 1..23: mol_type = other DNA
ggagcactatgaaaggaagctgg

SEQ ID NO: 894 - DNA, 23 bp, Homo sapiens
  source 1..23: mol_type = other DNA
aaggtgggacatgagtactgtgg

SEQ ID NO: 895 - DNA, 23 bp, Homo sapiens
  source 1..23: mol_type = other DNA
cctcctgcatggaaacacaatgg

SEQ ID NO: 896 - DNA, 23 bp, Homo sapiens
  source 1..23: mol_type = other DNA
tgtcaccccatagcaaaaggtgg

SEQ ID NO: 897 - DNA, 23 bp, Homo sapiens
  source 1..23: mol_type = other DNA
gtcaccccatagcaaaaggtggg

SEQ ID NO: 898 - DNA, 23 bp, Homo sapiens
  source 1..23: mol_type = other DNA
ctgaagccacacccttcccgggg

SEQ ID NO: 899 - DNA, 23 bp, Homo sapiens
  source 1..23: mol_type = other DNA
tggagaagcttatcaagaagagg

SEQ ID NO: 900 - DNA, 23 bp, Homo sapiens
  source 1..23: mol_type = other DNA
atgtcccaccttttgctatgggg

SEQ ID NO: 901 - DNA, 23 bp, Homo sapiens
  source 1..23: mol_type = other DNA
ctatggggtgacagcttctgtgg

SEQ ID NO: 902 - DNA, 23 bp, Homo sapiens
  source 1..23: mol_type = other DNA
agctgtcaccccatagcaaaagg

SEQ ID NO: 903 - DNA, 23 bp, Homo sapiens
  source 1..23: mol_type = other DNA
gtttccatgcaggaggccccggg

SEQ ID NO: 904 - DNA, 23 bp, Homo sapiens
  source 1..23: mol_type = other DNA
ccattgtgtttccatgcaggagg

SEQ ID NO: 905 - DNA, 23 bp, Homo sapiens
  source 1..23: mol_type = other DNA
catgcaggaggccccgggaaggg

SEQ ID NO: 906 - DNA, 23 bp, Homo sapiens
  source 1..23: mol_type = other DNA
gctgaagccacacccttcccggg

SEQ ID NO: 907 - DNA, 23 bp, Homo sapiens
  source 1..23: mol_type = other DNA
catgtcccaccttttgctatggg

SEQ ID NO: 908 - DNA, 23 bp, Homo sapiens
  source 1..23: mol_type = other DNA
gtgccaccccatgaacctggagg

SEQ ID NO: 909 - DNA, 23 bp, Homo sapiens
  source 1..23: mol_type = other DNA
ggtcatgatgaagcccacggagg

SEQ ID NO: 910 - DNA, 23 bp, Homo sapiens
  source 1..23: mol_type = other DNA
ccttcaccaggccctactggagg

SEQ ID NO: 911 - DNA, 23 bp, Homo sapiens
  source 1..23: mol_type = other DNA
accagcccagattcacctggggg

SEQ ID NO: 912 - DNA, 23 bp, Homo sapiens
  source 1..23: mol_type = other DNA
tggaggaccaggaagcccagggg

SEQ ID NO: 913 - DNA, 23 bp, Homo sapiens
  source 1..23: mol_type = other DNA
ggaggtcatgatgaagcccacgg

SEQ ID NO: 914 - DNA, 23 bp, Homo sapiens
  source 1..23: mol_type = other DNA
tcaccaggacggagtcgccgtgg

SEQ ID NO: 915 - DNA, 23 bp, Homo sapiens
  source 1..23: mol_type = other DNA
actagatgcagccctgagtgggg

SEQ ID NO: 916 - DNA, 23 bp, Homo sapiens
  source 1..23: mol_type = other DNA
aagtcaggtaaagtatgggaagg

SEQ ID NO: 917 - DNA, 23 bp, Homo sapiens
  source 1..23: mol_type = other DNA
tcagcagcactagggtcccgggg

SEQ ID NO: 918 - DNA, 23 bp, Homo sapiens
  source 1..23: mol_type = other DNA
agggccccccctcaccaggacgg

SEQ ID NO: 919 - DNA, 23 bp, Homo sapiens
  source 1..23: mol_type = other DNA
ggaggaccaggaagcccaggggg

SEQ ID NO: 920 - DNA, 23 bp, Homo sapiens
  source 1..23: mol_type = other DNA
aagacattttgcggagcaggagg

SEQ ID NO: 921 - DNA, 23 bp, Homo sapiens
  source 1..23: mol_type = other DNA
gggaaccacctcctccagtaggg

SEQ ID NO: 922 - DNA, 23 bp, Homo sapiens
  source 1..23: mol_type = other DNA
acctgctgtcagcagcactaggg

SEQ ID NO: 923 - DNA, 23 bp, Homo sapiens
  source 1..23: mol_type = other DNA
cccgaagtcaggtaaagtatggg

SEQ ID NO: 924 - DNA, 23 bp, Homo sapiens
  source 1..23: mol_type = other DNA
ttctgggaactggaagggaaggg

SEQ ID NO: 925 - DNA, 23 bp, Homo sapiens
  source 1..23: mol_type = other DNA
tggcaagacccgtcaccgcaagg

SEQ ID NO: 926 - DNA, 23 bp, Homo sapiens
  source 1..23: mol_type = other DNA
gtcatgatgaagcccacggaggg

SEQ ID NO: 927 - DNA, 23 bp, Homo sapiens
  source 1..23: mol_type = other DNA
ctttacctgacttcgggtgacgg

SEQ ID NO: 928 - DNA, 23 bp, Homo sapiens
  source 1..23: mol_type = other DNA
ccaccagcccagattcacctggg

SEQ ID NO: 929 - DNA, 23 bp, Homo sapiens
  source 1..23: mol_type = other DNA
gactccgtcctggtgaggggggg

SEQ ID NO: 930 - DNA, 23 bp, Homo sapiens
  source 1..23: mol_type = other DNA
aggaagccccaccccactcaggg

SEQ ID NO: 931 - DNA, 23 bp, Homo sapiens
  source 1..23: mol_type = other DNA
ggtcctccaggttcatggggtgg

SEQ ID NO: 932 - DNA, 23 bp, Homo sapiens
  source 1..23: mol_type = other DNA
ggctcaccacctccggcccgggg

SEQ ID NO: 933 - DNA, 23 bp, Homo sapiens
  source 1..23: mol_type = other DNA
cctccagtagggcctggtgaagg

SEQ ID NO: 934 - DNA, 23 bp, Homo sapiens
  source 1..23: mol_type = other DNA
tcaccgcaaggccagcgccaagg

SEQ ID NO: 935 - DNA, 23 bp, Homo sapiens
  source 1..23: mol_type = other DNA
gctgtgccaccccatgaacctgg

SEQ ID NO: 936 - DNA, 23 bp, Homo sapiens
  source 1..23: mol_type = other DNA
gggacgatgaagacattttgcgg

SEQ ID NO: 937 - DNA, 23 bp, Homo sapiens
  source 1..23: mol_type = other DNA
tgcggtgacgggtcttgccacgg

SEQ ID NO: 938 - DNA, 23 bp, Homo sapiens
  source 1..23: mol_type = other DNA
ggcacagctgtacgaagcccagg

SEQ ID NO: 939 - DNA, 23 bp, Homo sapiens
  source 1..23: mol_type = other DNA
cccagctgcactgctgtacaggg

SEQ ID NO: 940 - DNA, 23 bp, Homo sapiens
  source 1..23: mol_type = other DNA
attcacctgggggagccaaaggg

SEQ ID NO: 941 - DNA, 23 bp, Homo sapiens
  source 1..23: mol_type = other DNA
ccaggccctactggaggaggtgg

SEQ ID NO: 942 - DNA, 23 bp, Homo sapiens
  source 1..23: mol_type = other DNA
tcaagtgctgggacccagcccgg

SEQ ID NO: 943 - DNA, 23 bp, Homo sapiens
  source 1..23: mol_type = other DNA
gattcacctgggggagccaaagg

SEQ ID NO: 944 - DNA, 23 bp, Homo sapiens
  source 1..23: mol_type = other DNA
actagggtcccggggccgggggg

SEQ ID NO: 945 - DNA, 23 bp, Homo sapiens
  source 1..23: mol_type = other DNA
gggcttcctgggtgtcctaaggg

SEQ ID NO: 946 - DNA, 23 bp, Homo sapiens
  source 1..23: mol_type = other DNA
ggttcccctttggctcccccagg

SEQ ID NO: 947 - DNA, 23 bp, Homo sapiens
  source 1..23: mol_type = other DNA
tcccatactttacctgacttcgg

SEQ ID NO: 948 - DNA, 23 bp, Homo sapiens
  source 1..23: mol_type = other DNA
gtccccctcctaggccccctggg

SEQ ID NO: 949 - DNA, 23 bp, Homo sapiens
  source 1..23: mol_type = other DNA
cggcgactccgtcctggtgaggg

SEQ ID NO: 950 - DNA, 23 bp, Homo sapiens
  source 1..23: mol_type = other DNA
tcagggctgcatctagtttaggg

SEQ ID NO: 951 - DNA, 23 bp, Homo sapiens
  source 1..23: mol_type = other DNA
gtagggcctggtgaaggtgtggg

SEQ ID NO: 952 - DNA, 23 bp, Homo sapiens
  source 1..23: mol_type = other DNA
tagagctgacatcaagccagagg

SEQ ID NO: 953 - DNA, 23 bp, Homo sapiens
  source 1..23: mol_type = other DNA
atctcatcggaagaggaggaagg

SEQ ID NO: 954 - DNA, 23 bp, Homo sapiens
  source 1..23: mol_type = other DNA
gtaatgccatcacttacctctgg

SEQ ID NO: 955 - DNA, 23 bp, Homo sapiens
  source 1..23: mol_type = other DNA
gatggcattacaagaattaatgg

SEQ ID NO: 956 - DNA, 23 bp, Homo sapiens
  source 1..23: mol_type = other DNA
gctagtgaaccttcccccagtgg

SEQ ID NO: 957 - DNA, 23 bp, Homo sapiens
  source 1..23: mol_type = other DNA
gcagcaccaacactgatgagcgg

SEQ ID NO: 958 - DNA, 23 bp, Homo sapiens
  source 1..23: mol_type = other DNA
ctgatgagcggccagatgagcgg

SEQ ID NO: 959 - DNA, 23 bp, Homo sapiens
  source 1..23: mol_type = other DNA
agagaatccatcagatggggagg

SEQ ID NO: 960 - DNA, 23 bp, Homo sapiens
  source 1..23: mol_type = other DNA
ggatgacctctgaaggaggtggg

SEQ ID NO: 961 - DNA, 23 bp, Homo sapiens
  source 1..23: mol_type = other DNA
accaggaacttctcagagagcgg

SEQ ID NO: 962 - DNA, 23 bp, Homo sapiens
  source 1..23: mol_type = other DNA
ttcctggtgtggaatgggcaggg

SEQ ID NO: 963 - DNA, 23 bp, Homo sapiens
  source 1..23: mol_type = other DNA
ccaggaacttctcagagagcggg

SEQ ID NO: 964 - DNA, 23 bp, Homo sapiens
  source 1..23: mol_type = other DNA
ccagggatgacctctgaaggagg

SEQ ID NO: 965 - DNA, 23 bp, Homo sapiens
  source 1..23: mol_type = other DNA
ccagggctcagaaatcccactgg

SEQ ID NO: 966 - DNA, 23 bp, Homo sapiens
  source 1..23: mol_type = other DNA
tctgaaggaggtgggtccagtgg

SEQ ID NO: 967 - DNA, 23 bp, Homo sapiens
  source 1..23: mol_type = other DNA
cttctcagagagcgggtgagtgg

SEQ ID NO: 968 - DNA, 23 bp, Homo sapiens
  source 1..23: mol_type = other DNA
actggacccacctccttcagagg

SEQ ID NO: 969 - DNA, 23 bp, Homo sapiens
  source 1..23: mol_type = other DNA
gggatgacctctgaaggaggtgg

SEQ ID NO: 970 - DNA, 23 bp, Homo sapiens
  source 1..23: mol_type = other DNA
ctgaaggaggtgggtccagtggg

SEQ ID NO: 971 - DNA, 23 bp, Homo sapiens
  source 1..23: mol_type = other DNA
tctgatgacatgtgctcccaggg

SEQ ID NO: 972 - DNA, 23 bp, Homo sapiens
  source 1..23: mol_type = other DNA
atctggccgctcatcagtgttgg

SEQ ID NO: 973 - DNA, 23 bp, Homo sapiens
  source 1..23: mol_type = other DNA
gagaagttcctggtgtggaatgg

SEQ ID NO: 974 - DNA, 23 bp, Homo sapiens
  source 1..23: mol_type = other DNA
cccagtggcacacctgaagttgg

SEQ ID NO: 975 - DNA, 23 bp, Homo sapiens
  source 1..23: mol_type = other DNA
gtgccttcctccccatctgatgg

SEQ ID NO: 976 - DNA, 23 bp, Homo sapiens
  source 1..23: mol_type = other DNA
ccaacttcaggtgtgccactggg

SEQ ID NO: 977 - DNA, 23 bp, Homo sapiens
  source 1..23: mol_type = other DNA
cagggacttaaacagcaccccgg

SEQ ID NO: 978 - DNA, 23 bp, Homo sapiens
  source 1..23: mol_type = other DNA
acataatcaacagaaagatgggg

SEQ ID NO: 979 - DNA, 23 bp, Homo sapiens
  source 1..23: mol_type = other DNA
agccctgtcttaaggcccagggg

SEQ ID NO: 980 - DNA, 23 bp, Homo sapiens
  source 1..23: mol_type = other DNA
tggctcaggctgaagaaccgggg

SEQ ID NO: 981 - DNA, 23 bp, Homo sapiens
  source 1..23: mol_type = other DNA
ctatgtacaaggaatacgagtgg

SEQ ID NO: 982 - DNA, 23 bp, Homo sapiens
  source 1..23: mol_type = other DNA
caggcaccaggataaaagcaggg

SEQ ID NO: 983 - DNA, 23 bp, Homo sapiens
  source 1..23: mol_type = other DNA
ttatcaggtgaattggtcagggg

SEQ ID NO: 984 - DNA, 23 bp, Homo sapiens
  source 1..23: mol_type = other DNA
tatttcaaggtttttcacagggg

SEQ ID NO: 985 - DNA, 23 bp, Homo sapiens
  source 1..23: mol_type = other DNA
tctgagtcctcagaattgggagg

SEQ ID NO: 986 - DNA, 23 bp, Homo sapiens
  source 1..23: mol_type = other DNA
acaaggcaatagaaaagccaggg

SEQ ID NO: 987 - DNA, 23 bp, Homo sapiens
  source 1..23: mol_type = other DNA
ggcagctgtggaaatgaatgagg

SEQ ID NO: 988 - DNA, 23 bp, Homo sapiens
  source 1..23: mol_type = other DNA
gccctgtcttaaggcccaggggg

SEQ ID NO: 989 - DNA, 23 bp, Homo sapiens
  source 1..23: mol_type = other DNA
ccaacagcgttgatgccttgcgg

SEQ ID NO: 990 - DNA, 23 bp, Homo sapiens
  source 1..23: mol_type = other DNA
aatacgagtggctttcatggagg

SEQ ID NO: 991 - DNA, 23 bp, Homo sapiens
  source 1..23: mol_type = other DNA
gagtggctttcatggagggaggg

SEQ ID NO: 992 - DNA, 23 bp, Homo sapiens
  source 1..23: mol_type = other DNA
ctttatccccaaataataggggg

SEQ ID NO: 993 - DNA, 23 bp, Homo sapiens
  source 1..23: mol_type = other DNA
gctgaatataaggaatggggtgg

SEQ ID NO: 994 - DNA, 23 bp, Homo sapiens
  source 1..23: mol_type = other DNA
cttatattcagcaagcaacaagg

SEQ ID NO: 995 - DNA, 23 bp, Homo sapiens
  source 1..23: mol_type = other DNA
ctgagtcctcagaattgggaggg

SEQ ID NO: 996 - DNA, 23 bp, Homo sapiens
  source 1..23: mol_type = other DNA
aattcacctgataaactctaggg

SEQ ID NO: 997 - DNA, 23 bp, Homo sapiens
  source 1..23: mol_type = other DNA
acagaaaagtgcagtctaagtgg

SEQ ID NO: 998 - DNA, 23 bp, Homo sapiens
  source 1..23: mol_type = other DNA
atatttcaaggtttttcacaggg

SEQ ID NO: 999 - DNA, 23 bp, Homo sapiens
  source 1..23: mol_type = other DNA
tgaaatataacactccatgcagg

SEQ ID NO: 1000 - DNA, 23 bp, Homo sapiens
  source 1..23: mol_type = other DNA
cagccgtaagtcaggctcgaggg

SEQ ID NO: 1001 - DNA, 23 bp, Homo sapiens
  source 1..23: mol_type = other DNA
tctttatccccaaataatagggg

SEQ ID NO: 1002 - DNA, 23 bp, Homo sapiens
  source 1..23: mol_type = other DNA
ttacacattttgcacttgggagg

SEQ ID NO: 1003 - DNA, 23 bp, Homo sapiens
  source 1..23: mol_type = other DNA
atacgagtggctttcatggaggg

SEQ ID NO: 1004 - DNA, 23 bp, Homo sapiens
  source 1..23: mol_type = other DNA
tttttcacaggggttacagtagg

SEQ ID NO: 1005 - DNA, 23 bp, Homo sapiens
  source 1..23: mol_type = other DNA
ctgaatataaggaatggggtggg

SEQ ID NO: 1006 - DNA, 23 bp, Homo sapiens
  source 1..23: mol_type = other DNA
tctccctcgagcctgacttacgg

SEQ ID NO: 1007 - DNA, 23 bp, Homo sapiens
  source 1..23: mol_type = other DNA
aagccctgtcttaaggcccaggg

SEQ ID NO: 1008 - DNA, 23 bp, Homo sapiens
  source 1..23: mol_type = other DNA
tatccccaaataatagggggtgg

SEQ ID NO: 1009 - DNA, 23 bp, Homo sapiens
  source 1..23: mol_type = other DNA
cagtctcctaacctccccctggg

SEQ ID NO: 1010 - DNA, 23 bp, Homo sapiens
  source 1..23: mol_type = other DNA
tcaggcaccaggataaaagcagg

SEQ ID NO: 1011 - DNA, 23 bp, Homo sapiens
  source 1..23: mol_type = other DNA
tgaatataaggaatggggtgggg

SEQ ID NO: 1012 - DNA, 23 bp, Homo sapiens
  source 1..23: mol_type = other DNA
cccctctgaggtttctcaggtgg

SEQ ID NO: 1013 - DNA, 23 bp, Homo sapiens
  source 1..23: mol_type = other DNA
ctcgagcctgacttacggctggg

SEQ ID NO: 1014 - DNA, 23 bp, Homo sapiens
  source 1..23: mol_type = other DNA
gagggaagctgggggccgcaagg

SEQ ID NO: 1015 - DNA, 23 bp, Homo sapiens
  source 1..23: mol_type = other DNA
gcttgctgaatataaggaatggg

SEQ ID NO: 1016 - DNA, 23 bp, Homo sapiens
  source 1..23: mol_type = other DNA
tgcccttagccacagctctacgg

SEQ ID NO: 1017 - DNA, 23 bp, Homo sapiens
  source 1..23: mol_type = other DNA
tcagtctcctaacctccccctgg

SEQ ID NO: 1018 - DNA, 23 bp, Homo sapiens
  source 1..23: mol_type = other DNA
catgcaggcccagctgttgaggg

SEQ ID NO: 1019 - DNA, 23 bp, Homo sapiens
  source 1..23: mol_type = other DNA
tgggccttaagacagggcttggg

SEQ ID NO: 1020 - DNA, 23 bp, Homo sapiens
  source 1..23: mol_type = other DNA
gcatggagtgttatatttcaagg

SEQ ID NO: 1021 - DNA, 23 bp, Homo sapiens
  source 1..23: mol_type = other DNA
cctcgagcctgacttacggctgg

SEQ ID NO: 1022 - DNA, 23 bp, Homo sapiens
  source 1..23: mol_type = other DNA
gcttgggcagagaagataaatgg

SEQ ID NO: 1023 - DNA, 23 bp, Homo sapiens
  source 1..23: mol_type = other DNA
ttctttatccccaaataataggg

SEQ ID NO: 1024 - DNA, 23 bp, Homo sapiens
  source 1..23: mol_type = other DNA
gggagcctaggatccccgacagg

SEQ ID NO: 1025 - DNA, 23 bp, Homo sapiens
  source 1..23: mol_type = other DNA
aggaagttacgcacgggaggcgg

SEQ ID NO: 1026 - DNA, 23 bp, Homo sapiens
  source 1..23: mol_type = other DNA
aggcggtacctggttgtggaggg

SEQ ID NO: 1027 - DNA, 23 bp, Homo sapiens
  source 1..23: mol_type = other DNA
cagacgctccgccccacccatgg

SEQ ID NO: 1028 - DNA, 23 bp, Homo sapiens
  source 1..23: mol_type = other DNA
gaggcggtacctggttgtggagg

SEQ ID NO: 1029 - DNA, 23 bp, Homo sapiens
  source 1..23: mol_type = other DNA
ggttgtggagggtgacgccatgg

SEQ ID NO: 1030 - DNA, 23 bp, Homo sapiens
  source 1..23: mol_type = other DNA
cggagcgtctgggatgcgctggg

SEQ ID NO: 1031 - DNA, 23 bp, Homo sapiens
  source 1..23: mol_type = other DNA
gggtgacgccatgggtggggcgg

SEQ ID NO: 1032 - DNA, 23 bp, Homo sapiens
  source 1..23: mol_type = other DNA
ggagggtgacgccatgggtgggg

SEQ ID NO: 1033 - DNA, 23 bp, Homo sapiens
  source 1..23: mol_type = other DNA
acttcatttaaagaaacccacgg

SEQ ID NO: 1034 - DNA, 23 bp, Homo sapiens
  source 1..23: mol_type = other DNA
aaatcctagcactctgggagagg

SEQ ID NO: 1035 - DNA, 23 bp, Homo sapiens
  source 1..23: mol_type = other DNA
acatgagtcaatatgtattgtgg

SEQ ID NO: 1036 - DNA, 23 bp, Homo sapiens
  source 1..23: mol_type = other DNA
tcaggggaacagcaacacggtgg

SEQ ID NO: 1037 - DNA, 23 bp, Homo sapiens
  source 1..23: mol_type = other DNA
acacgatgaatcagagacggcgg

SEQ ID NO: 1038 - DNA, 23 bp, Homo sapiens
  source 1..23: mol_type = other DNA
aagatgagctcacagctatgggg

SEQ ID NO: 1039 - DNA, 23 bp, Homo sapiens
  source 1..23: mol_type = other DNA
aaaacctgagaaaaacaggaggg

SEQ ID NO: 1040 - DNA, 23 bp, Homo sapiens
  source 1..23: mol_type = other DNA
ggaacacgatgaatcagagacgg

SEQ ID NO: 1041 - DNA, 23 bp, Homo sapiens
  source 1..23: mol_type = other DNA
caaaacctgagaaaaacaggagg

SEQ ID NO: 1042 - DNA, 23 bp, Homo sapiens
  source 1..23: mol_type = other DNA
gtctcaggggaacagcaacacgg

SEQ ID NO: 1043 - DNA, 23 bp, Homo sapiens
  source 1..23: mol_type = other DNA
cgagcagcttgggagaagggtgg

SEQ ID NO: 1044 - DNA, 23 bp, Homo sapiens
  source 1..23: mol_type = other DNA
cagagttctgacttaagagaggg

SEQ ID NO: 1045 - DNA, 23 bp, Homo sapiens
  source 1..23: mol_type = other DNA
tcgaggaccagcaggaaaagggg

SEQ ID NO: 1046 - DNA, 23 bp, Homo sapiens
  source 1..23: mol_type = other DNA
gattacaaattgcagcagcaagg

SEQ ID NO: 1047 - DNA, 23 bp, Homo sapiens
  source 1..23: mol_type = other DNA
tggttatactcatggcacgatgg

SEQ ID NO: 1048 - DNA, 23 bp, Homo sapiens
  source 1..23: mol_type = other DNA
acagagttctgacttaagagagg

SEQ ID NO: 1049 - DNA, 23 bp, Homo sapiens
  source 1..23: mol_type = other DNA
tcaacactgttgtggtgacgggg

SEQ ID NO: 1050 - DNA, 23 bp, Homo sapiens
  source 1..23: mol_type = other DNA
agtgttgacgagcgtaagtgagg

SEQ ID NO: 1051 - DNA, 23 bp, Homo sapiens
  source 1..23: mol_type = other DNA
gtggacggagagagcacaagcgg

SEQ ID NO: 1052 - DNA, 23 bp, Homo sapiens
  source 1..23: mol_type = other DNA
tacgctcgtcaacactgttgtgg

SEQ ID NO: 1053 - DNA, 23 bp, Homo sapiens
  source 1..23: mol_type = other DNA
gagcgtaagtgaggattccaggg

SEQ ID NO: 1054 - DNA, 23 bp, Homo sapiens
  source 1..23: mol_type = other DNA
gaacagcgttcttcagctaaggg

SEQ ID NO: 1055 - DNA, 23 bp, Homo sapiens
  source 1..23: mol_type = other DNA
ctttcactgaagggtaagtgtgg

SEQ ID NO: 1056 - DNA, 23 bp, Homo sapiens
  source 1..23: mol_type = other DNA
gggaacagcaacacggtggacgg

SEQ ID NO: 1057 - DNA, 23 bp, Homo sapiens
  source 1..23: mol_type = other DNA
cagttcagcaggtcaggcagtgg

SEQ ID NO: 1058 - DNA, 23 bp, Homo sapiens
  source 1..23: mol_type = other DNA
gctcaggtgctcctgaaagttgg

SEQ ID NO: 1059 - DNA, 23 bp, Homo sapiens
  source 1..23: mol_type = other DNA
caagctgctcgaggaccagcagg

SEQ ID NO: 1060 - DNA, 23 bp, Homo sapiens
  source 1..23: mol_type = other DNA
gctgttctcaaactggtccctgg

SEQ ID NO: 1061 - DNA, 23 bp, Homo sapiens
  source 1..23: mol_type = other DNA
ggcctgtatggaatatcattggg

SEQ ID NO: 1062 - DNA, 23 bp, Homo sapiens
  source 1..23: mol_type = other DNA
agtgaaagcaaaaccttcaatgg

SEQ ID NO: 1063 - DNA, 23 bp, Homo sapiens
  source 1..23: mol_type = other DNA
gctcgaggaccagcaggaaaagg

SEQ ID NO: 1064 - DNA, 23 bp, Homo sapiens
  source 1..23: mol_type = other DNA
aggcctgtatggaatatcattgg

SEQ ID NO: 1065 - DNA, 23 bp, Homo sapiens
  source 1..23: mol_type = other DNA
gtaccatccccttttcctgctgg

SEQ ID NO: 1066 - DNA, 23 bp, Homo sapiens
  source 1..23: mol_type = other DNA
ggcagtggtgggtttcttgaagg

SEQ ID NO: 1067 - DNA, 23 bp, Homo sapiens
  source 1..23: mol_type = other DNA
cttcttgggcaagttccgggcgg

SEQ ID NO: 1068 - DNA, 23 bp, Homo sapiens
  source 1..23: mol_type = other DNA
gggtagtggagcccaaggagcgg

SEQ ID NO: 1069 - DNA, 23 bp, Homo sapiens
  source 1..23: mol_type = other DNA
agtgagagaacagaatgagacgg

SEQ ID NO: 1070 - DNA, 23 bp, Homo sapiens
  source 1..23: mol_type = other DNA
taacatcatcgcattcaagtagg

SEQ ID NO: 1071 - DNA, 23 bp, Homo sapiens
  source 1..23: mol_type = other DNA
gcttgtgtgcctagatacttggg

SEQ ID NO: 1072 - DNA, 23 bp, Homo sapiens
  source 1..23: mol_type = other DNA
tgatggccacctcttccgcccgg

SEQ ID NO: 1073 - DNA, 23 bp, Homo sapiens
  source 1..23: mol_type = other DNA
gagctgcagtggctgggtagtgg

SEQ ID NO: 1074 - DNA, 23 bp, Homo sapiens
  source 1..23: mol_type = other DNA
ctgttctctcactttcttgatgg

SEQ ID NO: 1075 - DNA, 23 bp, Homo sapiens
  source 1..23: mol_type = other DNA
tgcccatggacaactctacgagg

SEQ ID NO: 1076 - DNA, 23 bp, Homo sapiens
  source 1..23: mol_type = other DNA
catgataatacaataacatgggg

SEQ ID NO: 1077 - DNA, 23 bp, Homo sapiens
  source 1..23: mol_type = other DNA
gacctcgtagagttgtccatggg

SEQ ID NO: 1078 - DNA, 23 bp, Homo sapiens
  source 1..23: mol_type = other DNA
tggtccacaggaattgcaagtgg

SEQ ID NO: 1079 - DNA, 23 bp, Homo sapiens
  source 1..23: mol_type = other DNA
ccatgataatacaataacatggg

SEQ ID NO: 1080 - DNA, 23 bp, Homo sapiens
  source 1..23: mol_type = other DNA
tccatgataatacaataacatgg

SEQ ID NO: 1081 - DNA, 23 bp, Homo sapiens
  source 1..23: mol_type = other DNA
ttgctagtagactggtccacagg

SEQ ID NO: 1082 - DNA, 23 bp, Homo sapiens
  source 1..23: mol_type = other DNA
cattccacttgcaattcctgtgg

SEQ ID NO: 1083 - DNA, 23 bp, Homo sapiens
  source 1..23: mol_type = other DNA
acgaggtcttacgagctggcagg

SEQ ID NO: 1084 - DNA, 23 bp, Homo sapiens
  source 1..23: mol_type = other DNA
gatcacgatgaataattttatgg

SEQ ID NO: 1085 - DNA, 23 bp, Homo sapiens
  source 1..23: mol_type = other DNA
tttagtgacccacacagatgcgg

SEQ ID NO: 1086 - DNA, 23 bp, Homo sapiens
  source 1..23: mol_type = other DNA
aaatttttaccgcatctgtgtgg

SEQ ID NO: 1087 - DNA, 23 bp, Homo sapiens
  source 1..23: mol_type = other DNA
ggtcgcatggatggcgccagagg

SEQ ID NO: 1088 - DNA, 23 bp, Homo sapiens
  source 1..23: mol_type = other DNA
catttgctggcacggtcgcatgg

SEQ ID NO: 1089 - DNA, 23 bp, Homo sapiens
  source 1..23: mol_type = other DNA
ggatggcgccagaggtgatacgg

SEQ ID NO: 1090 - DNA, 23 bp, Homo sapiens
  source 1..23: mol_type = other DNA
ggttcattccgtatcacctctgg

SEQ ID NO: 1091 - DNA, 23 bp, Homo sapiens
  source 1..23: mol_type = other DNA
aaatccttatgaaacagacgtgg

SEQ ID NO: 1092 - DNA, 23 bp, Homo sapiens
  source 1..23: mol_type = other DNA
aagtggaaggaactggaaggtgg

SEQ ID NO: 1093 - DNA, 23 bp, Homo sapiens
  source 1..23: mol_type = other DNA
aagaatctacatctttgtaaggg

SEQ ID NO: 1094 - DNA, 23 bp, Homo sapiens
  source 1..23: mol_type = other DNA
aatccatcagggcaagtggaagg

SEQ ID NO: 1095 - DNA, 23 bp, Homo sapiens
  source 1..23: mol_type = other DNA
aaaagacctacagtagcgccggg

SEQ ID NO: 1096 - DNA, 23 bp, Homo sapiens
  source 1..23: mol_type = other DNA
gattcttcagccattatctgggg

SEQ ID NO: 1097 - DNA, 23 bp, Homo sapiens
  source 1..23: mol_type = other DNA
ctactgtaggtcttttggagtgg

SEQ ID NO: 1098 - DNA, 23 bp, Homo sapiens
  source 1..23: mol_type = other DNA
caaaagacctacagtagcgccgg

SEQ ID NO: 1099 - DNA, 23 bp, Homo sapiens
  source 1..23: mol_type = other DNA
tcagggcaagtggaaggaactgg

SEQ ID NO: 1100 - DNA, 23 bp, Homo sapiens
  source 1..23: mol_type = other DNA
ataaggattttgaatccatcagg

SEQ ID NO: 1101 - DNA, 23 bp, Homo sapiens
  source 1..23: mol_type = other DNA
gttccttccacttgccctgatgg

SEQ ID NO: 1102 - DNA, 23 bp, Homo sapiens
  source 1..23: mol_type = other DNA
gcttccaacaccccagataatgg

SEQ ID NO: 1103 - DNA, 23 bp, Homo sapiens
  source 1..23: mol_type = other DNA
gtgtctgccgaaaagaaggtcgg

SEQ ID NO: 1104 - DNA, 23 bp, Homo sapiens
  source 1..23: mol_type = other DNA
ggtggcaagtacatctgcagagg

SEQ ID NO: 1105 - DNA, 23 bp, Homo sapiens
  source 1..23: mol_type = other DNA
cttgaagtaagtttcttgtgggg

SEQ ID NO: 1106 - DNA, 23 bp, Homo sapiens
  source 1..23: mol_type = other DNA
gaagtaagtttcttgtggggtgg

SEQ ID NO: 1107 - DNA, 23 bp, Homo sapiens
  source 1..23: mol_type = other DNA
atgagtgtctgccgaaaagaagg

SEQ ID NO: 1108 - DNA, 23 bp, Homo sapiens
  source 1..23: mol_type = other DNA
agaaacttacttcaagtctcagg

SEQ ID NO: 1109 - DNA, 23 bp, Homo sapiens
  source 1..23: mol_type = other DNA
cgaaaagaaggtcggtttcgagg

SEQ ID NO: 1110 - DNA, 23 bp, Homo sapiens
  source 1..23: mol_type = other DNA
ctcgaaaccgaccttcttttcgg

SEQ ID NO: 1111 - DNA, 23 bp, Homo sapiens
  source 1..23: mol_type = other DNA
gtgaaggaacttgtatacaccgg

SEQ ID NO: 1112 - DNA, 23 bp, Homo sapiens
  source 1..23: mol_type = other DNA
tttgagaagatcaaaagtgaagg

SEQ ID NO: 1113 - DNA, 23 bp, Homo sapiens
  source 1..23: mol_type = other DNA
gaaggcgcagagaagagctcagg

SEQ ID NO: 1114 - DNA, 23 bp, Homo sapiens
  source 1..23: mol_type = other DNA
gaatcagttcttcatctaaccgg

SEQ ID NO: 1115 - DNA, 23 bp, Homo sapiens
  source 1..23: mol_type = other DNA
ttgtccttacccaggctgaatgg

SEQ ID NO: 1116 - DNA, 23 bp, Homo sapiens
  source 1..23: mol_type = other DNA
cttcttctctccattcagcctgg

SEQ ID NO: 1117 - DNA, 23 bp, Homo sapiens
  source 1..23: mol_type = other DNA
tgcctgtcagagaaaacacaggg

SEQ ID NO: 1118 - DNA, 23 bp, Homo sapiens
  source 1..23: mol_type = other DNA
atattcgtgaacactatgagcgg

SEQ ID NO: 1119 - DNA, 23 bp, Homo sapiens
  source 1..23: mol_type = other DNA
actatgagcggaagcttgagcgg

SEQ ID NO: 1120 - DNA, 23 bp, Homo sapiens
  source 1..23: mol_type = other DNA
tcatgctgcagctagaaatgcgg

SEQ ID NO: 1121 - DNA, 23 bp, Homo sapiens
  source 1..23: mol_type = other DNA
gcgggcgaataatttatacatgg

SEQ ID NO: 1122 - DNA, 23 bp, Homo sapiens
  source 1..23: mol_type = other DNA
ctatgagcggaagcttgagcggg

SEQ ID NO: 1123 - DNA, 23 bp, Homo sapiens
  source 1..23: mol_type = other DNA
catttctagctgcagcatgatgg

SEQ ID NO: 1124 - DNA, 23 bp, Homo sapiens
  source 1..23: mol_type = other DNA
tttctctgacaggcatgcgctgg

SEQ ID NO: 1125 - DNA, 23 bp, Homo sapiens
  source 1..23: mol_type = other DNA
gatggatgataggacgaacaggg

SEQ ID NO: 1126 - DNA, 23 bp, Homo sapiens
  source 1..23: mol_type = other DNA
ctgcaggcgtgagcaagcagtgg

SEQ ID NO: 1127 - DNA, 23 bp, Homo sapiens
  source 1..23: mol_type = other DNA
tatcatccatcccaatgccatgg

SEQ ID NO: 1128 - DNA, 23 bp, Homo sapiens
  source 1..23: mol_type = other DNA
gtttctccatggcattgggatgg

SEQ ID NO: 1129 - DNA, 23 bp, Homo sapiens
  source 1..23: mol_type = other DNA
gagaaactcatgaaaaggaaagg

SEQ ID NO: 1130 - DNA, 23 bp, Homo sapiens
  source 1..23: mol_type = other DNA
aatctgggatgcagaccaaacgg

SEQ ID NO: 1131 - DNA, 23 bp, Homo sapiens
  source 1..23: mol_type = other DNA
ccatggagaaactcatgaaaagg

SEQ ID NO: 1132 - DNA, 23 bp, Homo sapiens
  source 1..23: mol_type = other DNA
ggatggatgataggacgaacagg

SEQ ID NO: 1133 - DNA, 23 bp, Homo sapiens
  source 1..23: mol_type = other DNA
ccttttcatgagtttctccatgg

SEQ ID NO: 1134 - DNA, 23 bp, Homo sapiens
  source 1..23: mol_type = other DNA
tgctcacgcctgcagaataaagg

SEQ ID NO: 1135 - DNA, 23 bp, Homo sapiens
  source 1..23: mol_type = other DNA
cagacttgttgagatcagaaggg

SEQ ID NO: 1136 - DNA, 23 bp, Homo sapiens
  source 1..23: mol_type = other DNA
acggaagaaaatgaattcagcgg

SEQ ID NO: 1137 - DNA, 23 bp, Homo sapiens
  source 1..23: mol_type = other DNA
caacctgcgccaacaacctgagg

SEQ ID NO: 1138 - DNA, 23 bp, Homo sapiens
  source 1..23: mol_type = other DNA
catcccagactcaatatgcacgg

SEQ ID NO: 1139 - DNA, 23 bp, Homo sapiens
  source 1..23: mol_type = other DNA
agaagttctgagcctgacaaggg

SEQ ID NO: 1140 - DNA, 23 bp, Homo sapiens
  source 1..23: mol_type = other DNA
tttgggacttccggacaaagggg

SEQ ID NO: 1141 - DNA, 23 bp, Homo sapiens
  source 1..23: mol_type = other DNA
tgtaggtctgagtcatccctcgg

SEQ ID NO: 1142 - DNA, 23 bp, Homo sapiens
  source 1..23: mol_type = other DNA
tgcctctgctattccctcggcgg

SEQ ID NO: 1143 - DNA, 23 bp, Homo sapiens
  source 1..23: mol_type = other DNA
ataccctctgctgagccagtggg

SEQ ID NO: 1144 - DNA, 23 bp, Homo sapiens
  source 1..23: mol_type = other DNA
caccgccgagggaatagcagagg

SEQ ID NO: 1145 - DNA, 23 bp, Homo sapiens
  source 1..23: mol_type = other DNA
cagcgctacctcgaaaaacaagg

SEQ ID NO: 1146 - DNA, 23 bp, Homo sapiens
  source 1..23: mol_type = other DNA
tgtccgtgcatattgagtctggg

SEQ ID NO: 1147 - DNA, 23 bp, Homo sapiens
  source 1..23: mol_type = other DNA
agtgcaggccagaacagtatggg

SEQ ID NO: 1148 - DNA, 23 bp, Homo sapiens
  source 1..23: mol_type = other DNA
catgctcagagacagctgcccgg

SEQ ID NO: 1149 - DNA, 23 bp, Homo sapiens
  source 1..23: mol_type = other DNA
agcaaaccacgccaccgccgagg

SEQ ID NO: 1150 - DNA, 23 bp, Homo sapiens
  source 1..23: mol_type = other DNA
ctctgagcatggttgctgagtgg

SEQ ID NO: 1151 - DNA, 23 bp, Homo sapiens
  source 1..23: mol_type = other DNA
aattatggtgatgaagagaaggg

SEQ ID NO: 1152 - DNA, 23 bp, Homo sapiens
  source 1..23: mol_type = other DNA
tggaggggtgccgagatgagagg

SEQ ID NO: 1153 - DNA, 23 bp, Homo sapiens
  source 1..23: mol_type = other DNA
gttgctgagtgggctccgcaggg

SEQ ID NO: 1154 - DNA, 23 bp, Homo sapiens
  source 1..23: mol_type = other DNA
gcaaaccacgccaccgccgaggg

SEQ ID NO: 1155 - DNA, 23 bp, Homo sapiens
  source 1..23: mol_type = other DNA
tgccgagatgagaggtgccgagg

SEQ ID NO: 1156 - DNA, 23 bp, Homo sapiens
  source 1..23: mol_type = other DNA
gggatggggtgaattttcctggg

SEQ ID NO: 1157 - DNA, 23 bp, Homo sapiens
  source 1..23: mol_type = other DNA
gccgagatgagaggtgccgaggg

SEQ ID NO: 1158 - DNA, 23 bp, Homo sapiens
  source 1..23: mol_type = other DNA
ccagacttgttgagatcagaagg

SEQ ID NO: 1159 - DNA, 23 bp, Homo sapiens
  source 1..23: mol_type = other DNA
ctattccctcggcggtggcgtgg

SEQ ID NO: 1160 - DNA, 23 bp, Homo sapiens
  source 1..23: mol_type = other DNA
gatgaggtcagggctcgagccgg

SEQ ID NO: 1161 - DNA, 23 bp, Homo sapiens
  source 1..23: mol_type = other DNA
ctgctgggccgaaatacctcagg

SEQ ID NO: 1162 - DNA, 23 bp, Homo sapiens
  source 1..23: mol_type = other DNA
tctccagcagtctgcagccatgg

SEQ ID NO: 1163 - DNA, 23 bp, Homo sapiens
  source 1..23: mol_type = other DNA
ggctgcctctgctattccctcgg

SEQ ID NO: 1164 - DNA, 23 bp, Homo sapiens
  source 1..23: mol_type = other DNA
agattgcggcaaagtcactatgg

SEQ ID NO: 1165 - DNA, 23 bp, Homo sapiens
  source 1..23: mol_type = other DNA
gggatggtgactctgggacatgg

SEQ ID NO: 1166 - DNA, 23 bp, Homo sapiens
  source 1..23: mol_type = other DNA
attgggattgagcttggtgcagg

SEQ ID NO: 1167 - DNA, 23 bp, Homo sapiens
  source 1..23: mol_type = other DNA
tgacctcatctccacagccatgg

SEQ ID NO: 1168 - DNA, 23 bp, Homo sapiens
  source 1..23: mol_type = other DNA
gatctcaacaagtctggcctggg

SEQ ID NO: 1169 - DNA, 23 bp, Homo sapiens
  source 1..23: mol_type = other DNA
tttttcgaggtagcgctggaggg

SEQ ID NO: 1170 - DNA, 23 bp, Homo sapiens
  source 1..23: mol_type = other DNA
gccgaaatacctcaggttgttgg

SEQ ID NO: 1171 - DNA, 23 bp, Homo sapiens
  source 1..23: mol_type = other DNA
ttttgggacttccggacaaaggg

SEQ ID NO: 1172 - DNA, 23 bp, Homo sapiens
  source 1..23: mol_type = other DNA
atacctcaggttgttggcgcagg

SEQ ID NO: 1173 - DNA, 23 bp, Homo sapiens
  source 1..23: mol_type = other DNA
tctggcctgcactgaaggcaggg

SEQ ID NO: 1174 - DNA, 23 bp, Homo sapiens
  source 1..23: mol_type = other DNA
ttgaatttccacgaagacagagg

SEQ ID NO: 1175 - DNA, 23 bp, Homo sapiens
  source 1..23: mol_type = other DNA
tggagatgactcctcagaagagg

SEQ ID NO: 1176 - DNA, 23 bp, Homo sapiens
  source 1..23: mol_type = other DNA
tctccactctgaggagaacaagg

SEQ ID NO: 1177 - DNA, 23 bp, Homo sapiens
  source 1..23: mol_type = other DNA
ttccccttcttcctcttctgagg

SEQ ID NO: 1178 - DNA, 23 bp, Homo sapiens
  source 1..23: mol_type = other DNA
gagacatctgagtcttcacacgg

SEQ ID NO: 1179 - DNA, 23 bp, Homo sapiens
  source 1..23: mol_type = other DNA
cgtctagcttctctgccaagcgg

SEQ ID NO: 1180 - DNA, 23 bp, Homo sapiens
  source 1..23: mol_type = other DNA
cagctgctgatacagcgatgggg

SEQ ID NO: 1181 - DNA, 23 bp, Homo sapiens
  source 1..23: mol_type = other DNA
actcatcaggactgtttgagtgg

SEQ ID NO: 1182 - DNA, 23 bp, Homo sapiens
  source 1..23: mol_type = other DNA
gcctagaaccaagaagaaccagg

SEQ ID NO: 1183 - DNA, 23 bp, Homo sapiens
  source 1..23: mol_type = other DNA
cattgacatatcctcacactcgg

SEQ ID NO: 1184 - DNA, 23 bp, Homo sapiens
  source 1..23: mol_type = other DNA
ctgagtcttcacacggcgcacgg

SEQ ID NO: 1185 - DNA, 23 bp, Homo sapiens
  source 1..23: mol_type = other DNA
tctgtgtctgatggagaagaggg

SEQ ID NO: 1186 - DNA, 23 bp, Homo sapiens
  source 1..23: mol_type = other DNA
acatatcctcacactcggatggg

SEQ ID NO: 1187 - DNA, 23 bp, Homo sapiens
  source 1..23: mol_type = other DNA
tgataaacttgaagaccgcttgg

SEQ ID NO: 1188 - DNA, 23 bp, Homo sapiens
  source 1..23: mol_type = other DNA
ggaggaacgtggctatgaggtgg

SEQ ID NO: 1189 - DNA, 23 bp, Homo sapiens
  source 1..23: mol_type = other DNA
gagtgtgaggatatgtcaatggg

SEQ ID NO: 1190 - DNA, 23 bp, Homo sapiens
  source 1..23: mol_type = other DNA
gacatatcctcacactcggatgg

SEQ ID NO: 1191 - DNA, 23 bp, Homo sapiens
  source 1..23: mol_type = other DNA
ggcagctgctgatacagcgatgg

SEQ ID NO: 1192 - DNA, 23 bp, Homo sapiens
  source 1..23: mol_type = other DNA
cgagtgtgaggatatgtcaatgg

SEQ ID NO: 1193 - DNA, 23 bp, Homo sapiens
  source 1..23: mol_type = other DNA
gcagctgctgatacagcgatggg

SEQ ID NO: 1194 - DNA, 23 bp, Homo sapiens
  source 1..23: mol_type = other DNA
atgcaatcagtaaaacccagagg

SEQ ID NO: 1195 - DNA, 23 bp, Homo sapiens
  source 1..23: mol_type = other DNA
gatcactcagttcagctacgagg

SEQ ID NO: 1196 - DNA, 23 bp, Homo sapiens
  source 1..23: mol_type = other DNA
ataaaggtgggagaacaaagggg

SEQ ID NO: 1197 - DNA, 23 bp, Homo sapiens
  source 1..23: mol_type = other DNA
aggatgccaatgttccacagtgg

SEQ ID NO: 1198 - DNA, 23 bp, Homo sapiens
  source 1..23: mol_type = other DNA
aaaaactgatcaaaattacgagg

SEQ ID NO: 1199 - DNA, 23 bp, Homo sapiens
  source 1..23: mol_type = other DNA
gagaggaagaacatctacagtgg

SEQ ID NO: 1200 - DNA, 23 bp, Homo sapiens
  source 1..23: mol_type = other DNA
aaatgctccaggttccacaaggg

SEQ ID NO: 1201 - DNA, 23 bp, Homo sapiens
  source 1..23: mol_type = other DNA
acgaatccatgagccctgtgcgg

SEQ ID NO: 1202 - DNA, 23 bp, Homo sapiens
  source 1..23: mol_type = other DNA
tccactatttgacatcctggggg

SEQ ID NO: 1203 - DNA, 23 bp, Homo sapiens
  source 1..23: mol_type = other DNA
gacagtacaggagaccagagggg

SEQ ID NO: 1204 - DNA, 23 bp, Homo sapiens
  source 1..23: mol_type = other DNA
aacctggctccagagaggggtgg

SEQ ID NO: 1205 - DNA, 23 bp, Homo sapiens
  source 1..23: mol_type = other DNA
ctgacagtacaggagaccagagg

SEQ ID NO: 1206 - DNA, 23 bp, Homo sapiens
  source 1..23: mol_type = other DNA
gactgaactaggctaagcagagg

SEQ ID NO: 1207 - DNA, 23 bp, Homo sapiens
  source 1..23: mol_type = other DNA
tcaaaattacgaggtatcagggg

SEQ ID NO: 1208 - DNA, 23 bp, Homo sapiens
  source 1..23: mol_type = other DNA
cctgtaggaacaaaggttggggg

SEQ ID NO: 1209 - DNA, 23 bp, Homo sapiens
  source 1..23: mol_type = other DNA
cacagctaagatcaataccaggg

SEQ ID NO: 1210 - DNA, 23 bp, Homo sapiens
  source 1..23: mol_type = other DNA
gtggcaacagagaaacatgaggg

SEQ ID NO: 1211 - DNA, 23 bp, Homo sapiens
  source 1..23: mol_type = other DNA
ctgaaggtgaagagccactgggg

SEQ ID NO: 1212 - DNA, 23 bp, Homo sapiens
  source 1..23: mol_type = other DNA
cattgccgttcaatacagagagg

SEQ ID NO: 1213 - DNA, 23 bp, Homo sapiens
  source 1..23: mol_type = other DNA
caacactcttcagatgactgtgg

SEQ ID NO: 1214 - DNA, 23 bp, Homo sapiens
  source 1..23: mol_type = other DNA
tcttgcccatatcaccccagtgg

SEQ ID NO: 1215 - DNA, 23 bp, Homo sapiens
  source 1..23: mol_type = other DNA
agtggcaacagagaaacatgagg

SEQ ID NO: 1216 - DNA, 23 bp, Homo sapiens
  source 1..23: mol_type = other DNA
agaaggtgtgatttatgatgtgg

SEQ ID NO: 1217 - DNA, 23 bp, Homo sapiens
  source 1..23: mol_type = other DNA
attgccgttcaatacagagaggg

SEQ ID NO: 1218 - DNA, 23 bp, Homo sapiens
  source 1..23: mol_type = other DNA
acaggtctaaacttaagtggtgg

SEQ ID NO: 1219 - DNA, 23 bp, Homo sapiens
  source 1..23: mol_type = other DNA
tggtgttaggaaaacgaacgtgg

SEQ ID NO: 1220 - DNA, 23 bp, Homo sapiens
  source 1..23: mol_type = other DNA
agtctaagaagcaagtaactggg

SEQ ID NO: 1221 - DNA, 23 bp, Homo sapiens
  source 1..23: mol_type = other DNA
cagttccaggaagttcaagtggg

SEQ ID NO: 1222 - DNA, 23 bp, Homo sapiens
  source 1..23: mol_type = other DNA
tctcctagtggagagtagcaggg

SEQ ID NO: 1223 - DNA, 23 bp, Homo sapiens
  source 1..23: mol_type = other DNA
agtatgaaagatcaacacgttgg

SEQ ID NO: 1224 - DNA, 23 bp, Homo sapiens
  source 1..23: mol_type = other DNA
ctgcaaaagcctattcaagtcgg

SEQ ID NO: 1225 - DNA, 23 bp, Homo sapiens
  source 1..23: mol_type = other DNA
gcctgctgaccacttcctgtagg

SEQ ID NO: 1226 - DNA, 23 bp, Homo sapiens
  source 1..23: mol_type = other DNA
tattgcagctcatttctaggggg

SEQ ID NO: 1227 - DNA, 23 bp, Homo sapiens
  source 1..23: mol_type = other DNA
catttcaatcaagaatggcaggg

SEQ ID NO: 1228 - DNA, 23 bp, Homo sapiens
  source 1..23: mol_type = other DNA
agagtccagtgaatctagagagg

SEQ ID NO: 1229 - DNA, 23 bp, Homo sapiens
  source 1..23: mol_type = other DNA
ctgtgtacaacaatcatctgagg

SEQ ID NO: 1230 - DNA, 23 bp, Homo sapiens
  source 1..23: mol_type = other DNA
tgatttatgatgtggcacaatgg

SEQ ID NO: 1231 - DNA, 23 bp, Homo sapiens
  source 1..23: mol_type = other DNA
tcaaggtagcacatttgccaagg

SEQ ID NO: 1232 - DNA, 23 bp, Homo sapiens
  source 1..23: mol_type = other DNA
actgtaataaagcgtcaaagagg

SEQ ID NO: 1233 - DNA, 23 bp, Homo sapiens
  source 1..23: mol_type = other DNA
acaaaatgctgggagagagtggg

SEQ ID NO: 1234 - DNA, 23 bp, Homo sapiens
  source 1..23: mol_type = other DNA
gggctctgaaaagcattccaagg

SEQ ID NO: 1235 - DNA, 23 bp, Homo sapiens
  source 1..23: mol_type = other DNA
ctgtaataaagcgtcaaagaggg

SEQ ID NO: 1236 - DNA, 23 bp, Homo sapiens
  source 1..23: mol_type = other DNA
cttgcacccaatgtcttcaaggg

SEQ ID NO: 1237 - DNA, 23 bp, Homo sapiens
  source 1..23: mol_type = other DNA
gtcatgtggccagggtacgatgg

SEQ ID NO: 1238 - DNA, 23 bp, Homo sapiens
  source 1..23: mol_type = other DNA
acttcccagactctggaagtggg

SEQ ID NO: 1239 - DNA, 23 bp, Homo sapiens
  source 1..23: mol_type = other DNA
tgccgggagcttccttacagtgg

SEQ ID NO: 1240 - DNA, 23 bp, Homo sapiens
  source 1..23: mol_type = other DNA
ctgtaggaacaaaggttgggggg

SEQ ID NO: 1241 - DNA, 23 bp, Homo sapiens
  source 1..23: mol_type = other DNA
ttcattcgctccaagtcgatagg

SEQ ID NO: 1242 - DNA, 23 bp, Homo sapiens
  source 1..23: mol_type = other DNA
catggacattcatcccaagatgg

SEQ ID NO: 1243 - DNA, 23 bp, Homo sapiens
  source 1..23: mol_type = other DNA
tttacattgccaaaagcatgagg

SEQ ID NO: 1244 - DNA, 23 bp, Homo sapiens
  source 1..23: mol_type = other DNA
agattttcccggttatcacaagg

SEQ ID NO: 1245 - DNA, 23 bp, Homo sapiens
  source 1..23: mol_type = other DNA
tctccctgctactctccactagg

SEQ ID NO: 1246 - DNA, 23 bp, Homo sapiens
  source 1..23: mol_type = other DNA
cccggccccacaaattcccaagg

SEQ ID NO: 1247 - DNA, 23 bp, Homo sapiens
  source 1..23: mol_type = other DNA
accacttcctgtaggaacaaagg

SEQ ID NO: 1248 - DNA, 23 bp, Homo sapiens
  source 1..23: mol_type = other DNA
taaaaggaactgttatccagagg

SEQ ID NO: 1249 - DNA, 23 bp, Homo sapiens
  source 1..23: mol_type = other DNA
tgatcatgaaagctcctgggtgg

SEQ ID NO: 1250 - DNA, 23 bp, Homo sapiens
  source 1..23: mol_type = other DNA
tctaatgtgccagtctgtgaggg

SEQ ID NO: 1251 - DNA, 23 bp, Homo sapiens
  source 1..23: mol_type = other DNA
ccgattcttcaaactgcatgggg

SEQ ID NO: 1252 - DNA, 23 bp, Homo sapiens
  source 1..23: mol_type = other DNA
gcacactctactgcagagcaggg

SEQ ID NO: 1253 - DNA, 23 bp, Homo sapiens
  source 1..23: mol_type = other DNA
gatcatgaaagctcctgggtggg

SEQ ID NO: 1254 - DNA, 23 bp, Homo sapiens
  source 1..23: mol_type = other DNA
agagccctaagcagatggcaagg

SEQ ID NO: 1255 - DNA, 23 bp, Homo sapiens
  source 1..23: mol_type = other DNA
aacatccttgggaatttgtgggg

SEQ ID NO: 1256 - DNA, 23 bp, Homo sapiens
  source 1..23: mol_type = other DNA
atcagtaaaacccagaggagcgg

SEQ ID NO: 1257 - DNA, 23 bp, Homo sapiens
  source 1..23: mol_type = other DNA
acacagctaagatcaataccagg

SEQ ID NO: 1258 - DNA, 23 bp, Homo sapiens
  source 1..23: mol_type = other DNA
gagcatttctcagggacaggtgg

SEQ ID NO: 1259 - DNA, 23 bp, Homo sapiens
  source 1..23: mol_type = other DNA
ggctccctctctgtattgaacgg

SEQ ID NO: 1260 - DNA, 23 bp, Homo sapiens
  source 1..23: mol_type = other DNA
cttgaaatcacagtcttgtgagg

SEQ ID NO: 1261 - DNA, 23 bp, Homo sapiens
  source 1..23: mol_type = other DNA
gaaaacaacactgggagctgggg

SEQ ID NO: 1262 - DNA, 23 bp, Homo sapiens
  source 1..23: mol_type = other DNA
ggatgccaatgttccacagtggg

SEQ ID NO: 1263 - DNA, 23 bp, Homo sapiens
  source 1..23: mol_type = other DNA
ccggcatcagaaaacaacactgg

SEQ ID NO: 1264 - DNA, 23 bp, Homo sapiens
  source 1..23: mol_type = other DNA
tattattgctgcttgaacagggg

SEQ ID NO: 1265 - DNA, 23 bp, Homo sapiens
  source 1..23: mol_type = other DNA
actaaagaactttaagaaggagg

SEQ ID NO: 1266 - DNA, 23 bp, Homo sapiens
  source 1..23: mol_type = other DNA
ctcgcactagtgtccacctgtgg

SEQ ID NO: 1267 - DNA, 23 bp, Homo sapiens
  source 1..23: mol_type = other DNA
ggcatgtgcttcctctaggaggg

SEQ ID NO: 1268 - DNA, 23 bp, Homo sapiens
  source 1..23: mol_type = other DNA
gagaataaggtccttagtggagg

SEQ ID NO: 1269 - DNA, 23 bp, Homo sapiens
  source 1..23: mol_type = other DNA
tgggaatttgtggggccgggagg

SEQ ID NO: 1270 - DNA, 23 bp, Homo sapiens
  source 1..23: mol_type = other DNA
tctgaaggtgaagagccactggg

SEQ ID NO: 1271 - DNA, 23 bp, Homo sapiens
  source 1..23: mol_type = other DNA
tttaaaattgctttgcagagggg

SEQ ID NO: 1272 - DNA, 23 bp, Homo sapiens
  source 1..23: mol_type = other DNA
gggcatgtgcttcctctaggagg

SEQ ID NO: 1273 - DNA, 23 bp, Homo sapiens
  source 1..23: mol_type = other DNA
gttcaatacagagagggagccgg

SEQ ID NO: 1274 - DNA, 23 bp, Homo sapiens
  source 1..23: mol_type = other DNA
tctctgttgccactaaaggtggg

SEQ ID NO: 1275 - DNA, 23 bp, Homo sapiens
  source 1..23: mol_type = other DNA
agagcagagccctaagcagatgg

SEQ ID NO: 1276 - DNA, 23 bp, Homo sapiens
  source 1..23: mol_type = other DNA
tgttctgccctgaagcccaatgg

SEQ ID NO: 1277 - DNA, 23 bp, Homo sapiens
  source 1..23: mol_type = other DNA
caaggcaccatgaagtaccttgg

SEQ ID NO: 1278 - DNA, 23 bp, Homo sapiens
  source 1..23: mol_type = other DNA
caatgttccacagtgggagaggg

SEQ ID NO: 1279 - DNA, 23 bp, Homo sapiens
  source 1..23: mol_type = other DNA
cttttaaaattgctttgcagagg

SEQ ID NO: 1280 - DNA, 23 bp, Homo sapiens
  source 1..23: mol_type = other DNA
ccacctgtggctgtcaggggcgg

SEQ ID NO: 1281 - DNA, 23 bp, Homo sapiens
  source 1..23: mol_type = other DNA
gatccctgagtgctgtgaagtgg

SEQ ID NO: 1282 - DNA, 23 bp, Homo sapiens
  source 1..23: mol_type = other DNA
ataccagggagctcccaaatggg

SEQ ID NO: 1283 - DNA, 23 bp, Homo sapiens
  source 1..23: mol_type = other DNA
tcagttccaggaagttcaagtgg

SEQ ID NO: 1284 - DNA, 23 bp, Homo sapiens
  source 1..23: mol_type = other DNA
acttgatcatgaaagctcctggg

SEQ ID NO: 1285 - DNA, 23 bp, Homo sapiens
  source 1..23: mol_type = other DNA
ccaggatgtcaaatagtggatgg

SEQ ID NO: 1286 - DNA, 23 bp, Homo sapiens
  source 1..23: mol_type = other DNA
aattacgaggtatcaggggtagg

SEQ ID NO: 1287 - DNA, 23 bp, Homo sapiens
  source 1..23: mol_type = other DNA
aatttgagctcctatcgacttgg

SEQ ID NO: 1288 - DNA, 23 bp, Homo sapiens
  source 1..23: mol_type = other DNA
gaagtcctgccatcgtaccctgg

SEQ ID NO: 1289 - DNA, 23 bp, Homo sapiens
  source 1..23: mol_type = other DNA
gagttattgaataaaaacaaagg

SEQ ID NO: 1290 - DNA, 23 bp, Homo sapiens
  source 1..23: mol_type = other DNA
aagactaaagaactttaagaagg

SEQ ID NO: 1291 - DNA, 23 bp, Homo sapiens
  source 1..23: mol_type = other DNA
ttattgcagctcatttctagggg

SEQ ID NO: 1292 - DNA, 23 bp, Homo sapiens
  source 1..23: mol_type = other DNA
cggcatcagaaaacaacactggg

SEQ ID NO: 1293 - DNA, 23 bp, Homo sapiens
  source 1..23: mol_type = other DNA
ggtcactggacagcagcctaggg

SEQ ID NO: 1294 - DNA, 23 bp, Homo sapiens
  source 1..23: mol_type = other DNA
ggcttttgttaactgtcatgtgg

SEQ ID NO: 1295 - DNA, 23 bp, Homo sapiens
  source 1..23: mol_type = other DNA
aacatttgtggaaagaacaaggg

SEQ ID NO: 1296 - DNA, 23 bp, Homo sapiens
  source 1..23: mol_type = other DNA
gatgctgagcagggataggaagg

SEQ ID NO: 1297 - DNA, 23 bp, Homo sapiens
  source 1..23: mol_type = other DNA
cagtgagatctgaagtcaagagg

SEQ ID NO: 1298 - DNA, 23 bp, Homo sapiens
  source 1..23: mol_type = other DNA
actggtggtgtgagtatgctagg

SEQ ID NO: 1299 - DNA, 23 bp, Homo sapiens
  source 1..23: mol_type = other DNA
tttcccggttatcacaaggcagg

SEQ ID NO: 1300 - DNA, 23 bp, Homo sapiens
  source 1..23: mol_type = other DNA
ctccactaggagaataaaggtgg

SEQ ID NO: 1301 - DNA, 23 bp, Homo sapiens
  source 1..23: mol_type = other DNA
attgttgtccaaaagggcagtgg

SEQ ID NO: 1302 - DNA, 23 bp, Homo sapiens
  source 1..23: mol_type = other DNA
aagcaacctggctccagagaggg

SEQ ID NO: 1303 - DNA, 23 bp, Homo sapiens
  source 1..23: mol_type = other DNA
tatcaaagcaaacaaatgcatgg

SEQ ID NO: 1304 - DNA, 23 bp, Homo sapiens
  source 1..23: mol_type = other DNA
gaagtggtcagcaggcagaaagg

SEQ ID NO: 1305 - DNA, 23 bp, Homo sapiens
  source 1..23: mol_type = other DNA
tctttgacactgcatcatgttgg

SEQ ID NO: 1306 - DNA, 23 bp, Homo sapiens
  source 1..23: mol_type = other DNA
gctctgctacctggtaatgaagg

SEQ ID NO: 1307 - DNA, 23 bp, Homo sapiens
  source 1..23: mol_type = other DNA
gtatgaaagatcaacacgttggg

SEQ ID NO: 1308 - DNA, 23 bp, Homo sapiens
  source 1..23: mol_type = other DNA
ataccttctctagcccagcctgg

SEQ ID NO: 1309 - DNA, 23 bp, Homo sapiens
  source 1..23: mol_type = other DNA
actcaagtagacaagcaacctgg

SEQ ID NO: 1310 - DNA, 23 bp, Homo sapiens
  source 1..23: mol_type = other DNA
gaaggaacaccaatcactgatgg

SEQ ID NO: 1311 - skipped

SEQ ID NO: 1312 - DNA, 23 bp, Homo sapiens
  source 1..23: mol_type = other DNA
tcacttagaggaccaacgactgg

SEQ ID NO: 1313 - DNA, 23 bp, Homo sapiens
  source 1..23: mol_type = other DNA
agagccacttcacagcactcagg

SEQ ID NO: 1314 - DNA, 23 bp, Homo sapiens
  source 1..23: mol_type = other DNA
tgtccacctgtggctgtcagggg

SEQ ID NO: 1315 - DNA, 23 bp, Homo sapiens
  source 1..23: mol_type = other DNA
aactgcatggggttctgcaaagg

SEQ ID NO: 1316 - DNA, 23 bp, Homo sapiens
  source 1..23: mol_type = other DNA
ttctatccaacataaaacactgg

SEQ ID NO: 1317 - DNA, 23 bp, Homo sapiens
  source 1..23: mol_type = other DNA
gtattattgctgcttgaacaggg

SEQ ID NO: 1318 - DNA, 23 bp, Homo sapiens
  source 1..23: mol_type = other DNA
gaactgagtgatcctgagaatgg

SEQ ID NO: 1319 - DNA, 23 bp, Homo sapiens
  source 1..23: mol_type = other DNA
cccccccccaaaaaaagtctggg

SEQ ID NO: 1320 - DNA, 23 bp, Homo sapiens
  source 1..23: mol_type = other DNA
cggactgtgactcttcagatggg

SEQ ID NO: 1321 - DNA, 23 bp, Homo sapiens
  source 1..23: mol_type = other DNA
gtaccgcccctgacagccacagg

SEQ ID NO: 1322 - DNA, 23 bp, Homo sapiens
  source 1..23: mol_type = other DNA
tgagtcatattgattcaagcagg

SEQ ID NO: 1323 - DNA, 23 bp, Homo sapiens
  source 1..23: mol_type = other DNA
agtggcctttcccctttctgtgg

SEQ ID NO: 1324 - DNA, 23 bp, Homo sapiens
  source 1..23: mol_type = other DNA
aatctagagaggttgtttgacgg

SEQ ID NO: 1325 - DNA, 23 bp, Homo sapiens
  source 1..23: mol_type = other DNA
tgaggtcctgcctgtctccaggg

SEQ ID NO: 1326 - DNA, 23 bp, Homo sapiens
  source 1..23: mol_type = other DNA
tgacagtacaggagaccagaggg

SEQ ID NO: 1327 - DNA, 23 bp, Homo sapiens
  source 1..23: mol_type = other DNA
tctgtcttggagacagggacagg

SEQ ID NO: 1328 - DNA, 23 bp, Homo sapiens
  source 1..23: mol_type = other DNA
gcacatgcccttgaagacattgg

SEQ ID NO: 1329 - DNA, 23 bp, Homo sapiens
  source 1..23: mol_type = other DNA
gcttgggtttgggatttacctgg

SEQ ID NO: 1330 - DNA, 23 bp, Homo sapiens
  source 1..23: mol_type = other DNA
ggtcatttccgtaagccacctgg

SEQ ID NO: 1331 - DNA, 23 bp, Homo sapiens
  source 1..23: mol_type = other DNA
agaactttaagaaggaggcctgg

SEQ ID NO: 1332 - DNA, 23 bp, Homo sapiens
  source 1..23: mol_type = other DNA
agtgtcccaccctcacagactgg

SEQ ID NO: 1333 - DNA, 23 bp, Homo sapiens
  source 1..23: mol_type = other DNA
gttcagctacgaggagaagatgg

SEQ ID NO: 1334 - DNA, 23 bp, Homo sapiens
  source 1..23: mol_type = other DNA
ccaatgttccacagtgggagagg

SEQ ID NO: 1335 - DNA, 23 bp, Homo sapiens
  source 1..23: mol_type = other DNA
ttttaaaattgctttgcagaggg

SEQ ID NO: 1336 - DNA, 23 bp, Homo sapiens
  source 1..23: mol_type = other DNA
cctctcccactgtggaacattgg

SEQ ID NO: 1337 - DNA, 23 bp, Homo sapiens
  source 1..23: mol_type = other DNA
actgttgtaacacacttgtcagg

SEQ ID NO: 1338 - DNA, 23 bp, Homo sapiens
  source 1..23: mol_type = other DNA
agcaggagaacagactttgaagg

SEQ ID NO: 1339 - DNA, 23 bp, Homo sapiens
  source 1..23: mol_type = other DNA
tcggactgtgactcttcagatgg

SEQ ID NO: 1340 - DNA, 23 bp, Homo sapiens
  source 1..23: mol_type = other DNA
ctcttcccagacaaaatgctggg

SEQ ID NO: 1341 - DNA, 23 bp, Homo sapiens
  source 1..23: mol_type = other DNA
tccttagtggaggttaaaaaggg

SEQ ID NO: 1342 - DNA, 23 bp, Homo sapiens
  source 1..23: mol_type = other DNA
cttcaaccagcttgtccaggtgg

SEQ ID NO: 1343 - DNA, 23 bp, Homo sapiens
  source 1..23: mol_type = other DNA
attcccatttgggagctccctgg

SEQ ID NO: 1344 - DNA, 23 bp, Homo sapiens
  source 1..23: mol_type = other DNA
gtccgattcttcaaactgcatgg

SEQ ID NO: 1345 - DNA, 23 bp, Homo sapiens
  source 1..23: mol_type = other DNA
tagcttgcacatgctggctgggg

SEQ ID NO: 1346 - DNA, 23 bp, Homo sapiens
  source 1..23: mol_type = other DNA
ccttggcctgcagtcactggagg

SEQ ID NO: 1347 - DNA, 23 bp, Homo sapiens
  source 1..23: mol_type = other DNA
caaacaggtctaaacttaagtgg

SEQ ID NO: 1348 - DNA, 23 bp, Homo sapiens
  source 1..23: mol_type = other DNA
agacctgtttgcaagatggtggg

SEQ ID NO: 1349 - DNA, 23 bp, Homo sapiens
  source 1..23: mol_type = other DNA
tcccacctttattctcctagtgg

SEQ ID NO: 1350 - DNA, 23 bp, Homo sapiens
  source 1..23: mol_type = other DNA
cttttcagagcccatgaccctgg

SEQ ID NO: 1351 - DNA, 23 bp, Homo sapiens
  source 1..23: mol_type = other DNA
taactgttatgagtatgttaggg

SEQ ID NO: 1352 - DNA, 23 bp, Homo sapiens
  source 1..23: mol_type = other DNA
tttgacatcctgggggtcatcgg

SEQ ID NO: 1353 - DNA, 23 bp, Homo sapiens
  source 1..23: mol_type = other DNA
aaatggatcaaacaagcaaaagg

SEQ ID NO: 1354 - DNA, 23 bp, Homo sapiens
  source 1..23: mol_type = other DNA
ctgaacatttcaatcaagaatgg

SEQ ID NO: 1355 - DNA, 23 bp, Homo sapiens
  source 1..23: mol_type = other DNA
tttctgtttataaaaggtgaggg

SEQ ID NO: 1356 - DNA, 23 bp, Homo sapiens
  source 1..23: mol_type = other DNA
cagggacaggtggctaaagaggg

SEQ ID NO: 1357 - DNA, 23 bp, Homo sapiens
  source 1..23: mol_type = other DNA
attggaattgttgtccaaaaggg

SEQ ID NO: 1358 - DNA, 23 bp, Homo sapiens
  source 1..23: mol_type = other DNA
gtttagacctgtttgcaagatgg

SEQ ID NO: 1359 - DNA, 23 bp, Homo sapiens
  source 1..23: mol_type = other DNA
gaatgggatccatcagtgattgg

SEQ ID NO: 1360 - DNA, 23 bp, Homo sapiens
  source 1..23: mol_type = other DNA
cttcctgtaggaacaaaggttgg

SEQ ID NO: 1361 - DNA, 23 bp, Homo sapiens
  source 1..23: mol_type = other DNA
agtgttctgatgccacagttagg

SEQ ID NO: 1362 - DNA, 23 bp, Homo sapiens
  source 1..23: mol_type = other DNA
cagtactgaggaggagaaaaggg

SEQ ID NO: 1363 - DNA, 23 bp, Homo sapiens
  source 1..23: mol_type = other DNA
tctaggatgacatcacagttagg

SEQ ID NO: 1364 - DNA, 23 bp, Homo sapiens
  source 1..23: mol_type = other DNA
tctctgtattgaacggcaatggg

SEQ ID NO: 1365 - DNA, 23 bp, Homo sapiens
  source 1..23: mol_type = other DNA
aataccagggagctcccaaatgg

SEQ ID NO: 1366 - DNA, 23 bp, Homo sapiens
  source 1..23: mol_type = other DNA
tttcgagactttgacttgtacgg

SEQ ID NO: 1367 - DNA, 23 bp, Homo sapiens
  source 1..23: mol_type = other DNA
ctcattttctgtcgatcagaagg

SEQ ID NO: 1368 - DNA, 23 bp, Homo sapiens
  source 1..23: mol_type = other DNA
ggaaatgagctacaaggaaatgg

SEQ ID NO: 1369 - DNA, 23 bp, Homo sapiens
  source 1..23: mol_type = other DNA
tcaggggcggtaccacagaaagg

SEQ ID NO: 1370 - DNA, 23 bp, Homo sapiens
  source 1..23: mol_type = other DNA
ctctctgtattgaacggcaatgg

SEQ ID NO: 1371 - DNA, 23 bp, Homo sapiens
  source 1..23: mol_type = other DNA
tattcaagtcggactgaactagg

SEQ ID NO: 1372 - DNA, 23 bp, Homo sapiens
  source 1..23: mol_type = other DNA
gacagtgacagcactaaaaaggg

SEQ ID NO: 1373 - DNA, 23 bp, Homo sapiens
  source 1..23: mol_type = other DNA
tgcagctcatttctagggggtgg

SEQ ID NO: 1374 - DNA, 23 bp, Homo sapiens
  source 1..23: mol_type = other DNA
gcacatctattatttagaaaagg

SEQ ID NO: 1375 - DNA, 23 bp, Homo sapiens
  source 1..23: mol_type = other DNA
actctccactaggagaataaagg

SEQ ID NO: 1376 - DNA, 23 bp, Homo sapiens
  source 1..23: mol_type = other DNA
gcagtactgaggaggagaaaagg

SEQ ID NO: 1377 - DNA, 23 bp, Homo sapiens
  source 1..23: mol_type = other DNA
tatcttacatctttcagtgatgg

SEQ ID NO: 1378 - DNA, 23 bp, Homo sapiens
  source 1..23: mol_type = other DNA
aataaaggtgggagaacaaaggg

SEQ ID NO: 1379 - DNA, 23 bp, Homo sapiens
  source 1..23: mol_type = other DNA
agagagtgggatgagtctggggg

SEQ ID NO: 1380 - DNA, 23 bp, Homo sapiens
  source 1..23: mol_type = other DNA
ccagtgttgttttctgatgccgg

SEQ ID NO: 1381 - DNA, 23 bp, Homo sapiens
  source 1..23: mol_type = other DNA
cccccaacctttgttcctacagg

SEQ ID NO: 1382 - DNA, 23 bp, Homo sapiens
  source 1..23: mol_type = other DNA
gacaaaatgctgggagagagtgg

SEQ ID NO: 1383 - DNA, 23 bp, Homo sapiens
  source 1..23: mol_type = other DNA
gttattgcagctcatttctaggg

SEQ ID NO: 1384 - DNA, 23 bp, Homo sapiens
  source 1..23: mol_type = other DNA
agtgggactgttttaagaaaagg

SEQ ID NO: 1385 - DNA, 23 bp, Homo sapiens
  source 1..23: mol_type = other DNA
tctaaataatagatgtgcttggg

SEQ ID NO: 1386 - DNA, 23 bp, Homo sapiens
  source 1..23: mol_type = other DNA
aaggaggcctggtcacttagagg

SEQ ID NO: 1387 - DNA, 23 bp, Homo sapiens
  source 1..23: mol_type = other DNA
gccagcatgtgcaagctattagg

SEQ ID NO: 1388 - DNA, 23 bp, Homo sapiens
  source 1..23: mol_type = other DNA
agaggtttcagaggagaataagg

SEQ ID NO: 1389 - DNA, 23 bp, Homo sapiens
  source 1..23: mol_type = other DNA
aaaatgtctttacttctagaagg

SEQ ID NO: 1390 - DNA, 23 bp, Homo sapiens
  source 1..23: mol_type = other DNA
gctgcctgccttgtgataaccgg

SEQ ID NO: 1391 - DNA, 23 bp, Homo sapiens
  source 1..23: mol_type = other DNA
aatagcttgcacatgctggctgg

SEQ ID NO: 1392 - DNA, 23 bp, Homo sapiens
  source 1..23: mol_type = other DNA
gagcctgtcgttctgccattggg

SEQ ID NO: 1393 - DNA, 23 bp, Homo sapiens
  source 1..23: mol_type = other DNA
gtgattggtgttccttcaattgg

SEQ ID NO: 1394 - DNA, 23 bp, Homo sapiens
  source 1..23: mol_type = other DNA
ctttttttgggggggggtgaggg

SEQ ID NO: 1395 - DNA, 23 bp, Homo sapiens
  source 1..23: mol_type = other DNA
agtaaatcatttgatcaaaaagg

SEQ ID NO: 1396 - DNA, 23 bp, Homo sapiens
  source 1..23: mol_type = other DNA
gagtgggatgagtctgggggtgg

SEQ ID NO: 1397 - DNA, 23 bp, Homo sapiens
  source 1..23: mol_type = other DNA
actgattgcatcagccaaaaagg

SEQ ID NO: 1398 - DNA, 4662 bp, synthetic construct
  misc_feature 1..4662: note = Description of Artificial Sequence: Synthetic polynucleotide
  source 1..4662: mol_type = other DNA
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctagggcggccgctaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagatttacttatgtagctcgtccatgccgccggtggagtggcggccctcggcgcgttcgtactgttccacgatggtgtagtcctcgttgtgggaggtgatgtccaacttgatgttgacgttgtaggcgccgggcagctgcacgggcttcttggccttgtaggtggtcttgacctcagcgtcgtagtggccgccgtccttcagcttcagcctctgcttgatctcgcccttcagggcgccgtcctcggggtacatccgctcggaggaggcctcccagcccatggtcttcttctgcattacggggccgtcggaggggaagttggtgccgcgcagcttcaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcaccatggtggcgaccggtggatccttagagctagtgtacttggtaactgccttagtgccctcggacacagcatgcttagccagctccccaggcagcagcaggcgcacagccgtctgaatctccctggaggtgatggtcgagcgcttattgtagtgagccaggcgagaagcctcgcccgcgatgcgctcgaagatgtcgttgacgaaggagttcatgatccccatggccttggatgagatgccggtgtcggggtggacctgcttcagaaccttgtacacatagatagaatagctctccttgcggctgcgcttacgcttcttaccatccttcttctgcgccttagtgatagccttcttagaaccctttttaggggctggagcagacttagagggttcaggcatggtggcggccgctcttgaaggacgacgtcatcatcccttgcccggatgcgcgggcttcttgtctagcacaggagcctggggtagagcgcatgcaaattacgcgctgtgctttgtgggaaatcaccctaaacgaaaaatttattcctctttcgagccttatagtggcggccggtctacatcctgaagaagaaggttcgagatctcagttttagagctagaaatagcaagttaaaataaggctagtccgttatcaacttgaaaaagtggcaccgagtcggtgctttttttctagacccagctttcttgtacaaagttggcggccgcactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 1399 - DNA, 4671 bp, synthetic construct
  misc_feature 1..4671: note = Description of Artificial Sequence: Synthetic polynucleotide
  source 1..4671: mol_type = other DNA
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctagggcggccgctaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagatttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtggatccttagagctagtgtacttggtaactgccttagtgccctcggacacagcatgcttagccagctccccaggcagcagcaggcgcacagccgtctgaatctccctggaggtgatggtcgagcgcttattgtagtgagccaggcgagaagcctcgcccgcgatgcgctcgaagatgtcgttgacgaaggagttcatgatccccatggccttggatgagatgccggtgtcggggtggacctgcttcagaaccttgtacacatagatagaatagctctccttgcggctgcgcttacgcttcttaccatccttcttctgcgccttagtgatagccttcttagaaccctttttaggggctggagcagacttagagggttcaggcatggtggcggccgctcttgaaggacgacgtcatcatcccttgcccggatgcgcgggcttcttgtctagcacaggagcctggggtagagcgcatgcaaattacgcgctgtgctttgtgggaaatcaccctaaacgaaaaatttattcctctttcgagccttatagtggcggccggtctacatcctgaagccgacaccccaaatgatgggttttagagctagaaatagcaagttaaaataaggctagtccgttatcaacttgaaaaagtggcaccgagtcggtgctttttttctagacccagctttcttgtacaaagttggcggccgcactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 1400 - DNA, 4868 bp, synthetic construct
  misc_feature 1..4868: note = Description of Artificial Sequence: Synthetic polynucleotide
  source 1..4868: mol_type = other DNA
aaagcttcccggggggatctgggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacctagggcggccgcctgcaatatttgcatgtcgctatgtgttctgggaaatcaccataaacgtgaaatgtctttggatttgggagtcttataagttctgtatgagaccactttttcccgccgacaccccaaatgatgggttttagagctagaaatagcaagttaaaataaggctagtccgttatcaacttgaaaaagtggcaccgagtcggtgctttttttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatctagatttacttatgtagctcgtccatgccgccggtggagtggcggccctcggcgcgttcgtactgttccacgatggtgtagtcctcgttgtgggaggtgatgtccaacttgatgttgacgttgtaggcgccgggcagctgcacgggcttcttggccttgtaggtggtcttgacctcagcgtcgtagtggccgccgtccttcagcttcagcctctgcttgatctcgcccttcagggcgccgtcctcggggtacatccgctcggaggaggcctcccagcccatggtcttcttctgcattacggggccgtcggaggggaagttggtgccgcgcagcttcaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcaccatggtggcgaccggtggatccttagagctagtgtacttggtaactgccttagtgccctcggacacagcatgcttagccagctccccaggcagcagcaggcgcacagccgtctgaatctccctggaggtgatggtcgagcgcttattgtagtgagccaggcgagaagcctcgcccgcgatgcgctcgaagatgtcgttgacgaaggagttcatgatccccatggccttggatgagatgccggtgtcggggtggacctgcttcagaaccttgtacacatagatagaatagctctccttgcggctgcgcttacgcttcttaccatccttcttctgcgccttagtgatagccttcttagaaccctttttaggggctggagcagacttagagggttcaggcatggtggcggccgctcttgaaggacgacgtcatcatcccttgcccggatgcgcgggcttcttgtctagcacaggagcctggggtagagcgcatgcaaattacgcgctgtgctttgtgggaaatcaccctaaacgaaaaatttattcctctttcgagccttatagtggcggccggtctacatcctgaagaagaaggttcgagatctcagttttagagctagaaatagcaagttaaaataaggctagtccgttatcaacttgaaaaagtggcaccgagtcggtgctttttttctagacccagctttcttgtacaaagttggcggccgcactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctag

SEQ ID NO: 1401 - DNA, 201 bp, Tupaia belangeri
  source 1..201: mol_type = other DNA
gggggaagctgggtccactgagttcttataaggtttccagtcctagagcgattttaccattgcggtgatttcccagcatccgtagctacatgcaaatagcgcggggcgcgtctctcaggtccctccccgccctctcactgtacgtacccgcgtcctagggacgccgcgcccggggttcccggacgtcagcgttccgacgca

SEQ ID NO: 1402 - DNA, 76 bp, Homo sapiens
  source 1..76: mol_type = other DNA
gtgaaattgccctgtggtccttggtggtcctggccatcgagcggtacgtggtggtgtgtaagcccatgagcaactt

SEQ ID NO: 1403 - protein, 25 aa, Homo sapiens
EIALWSLVVLAIERYVVVCKPMSNF

SEQ ID NO: 1404 - RNA, 146 nt, synthetic construct
  misc_feature 1..146: note = Description of Artificial Sequence: Synthetic polynucleotide
  source 1..146: mol_type = other RNA
gggnnnnnnnnnnctgatgagtccgtgaggacgaaacgaggaaactcgtcnnnnnnnnnnnnnnnnnnnngttttagagctagaaatagcaagttaaaataaggctagtccgttatcaacttgaaaaagtggcaccgagtcggtgc

SEQ ID NO: 1405 - DNA, 482 bp, Mus musculus
  source 1..482: mol_type = other DNA
  CDS 1..135: protein_id = 1458; transl_table = 1; translation = LPAVACVLWSRLCQSLSRQGAGSQKDLQGSHCHSGTADVPHQLE L
  CDS 139..369: protein_id = 1460; transl_table = 1; translation = MLDSQVTQWLPLAHLYPAHVAGGHYWQKALGEALHLGRGWVLLH PAHREAQHLEVHIGWASGLSRGCSLKCLDPAFA
  CDS 373..387: protein_id = 1459; transl_table = 1; translation = KPHLE
  CDS 391..480: protein_id = 1461; transl_table = 1; translation = TQSPQRKGRVFGFHGGRPWPLEPGVLSSGP
cttcctgctgttgcttgtgttctgtggagtaggctttgccaatcattgtccagacagggcgcaggcagccaaaaagaccttcaaggaagccactgccactctggcactgcagacgtacctcatcagctcgaactctagatgctcgactctcaggtgacccagtggctccccctggcccacctgtatcctgctcatgtagctggaggacactattggcaaaaggctctgggggaggctctccacctggggaggggctgggtcctcctccaccctgcccaccgagaggcacaacatctcgaagtacacattgggtgggcgtcaggtctttctcgggggtgcagtctgaagtgtctggatccagctttcgcatagaagcctcacttagagtagacacaaagcccccaaaggaaggggagggtgttcgggtttcatggaggcaggccatggcctctggagccaggtgtgctgagcagcgggccctg

SEQ ID NO: 1406 - protein, 45 aa, Mus musculus
LPAVACVLWSRLCQSLSRQGAGSQKDLQGSHCHSGTADVPHQLEL

SEQ ID NO: 1407 - protein, 77 aa, Mus musculus
MLDSQVTQWLPLAHLYPAHVAGGHYWQKALGEALHLGRGWVLLHPAHREAQHLEVHIGWASGLSRGCSLKCLDPAFA

SEQ ID NO: 1408 - protein, 5 aa, Mus musculus
KPHLE

SEQ ID NO: 1409 - protein, 30 aa, Mus musculus
TQSPQRKGRVFGFHGGRPWPLEPGVLSSGP

SEQ ID NO: 1410 - skipped

SEQ ID NO: 1411 - DNA, 39 bp, Mus musculus
  source 1..39: mol_type = other DNA
cttgatgtcagtctccttgagatctcgaaccttcttcac

SEQ ID NO: 1412 - DNA, 39 bp, Mus musculus; identical to SEQ ID NO: 1411
  source 1..39: mol_type = other DNA

SEQ ID NO: 1413 - DNA, 39 bp, Mus musculus; identical to SEQ ID NO: 1411
  source 1..39: mol_type = other DNA

SEQ ID NO: 1414 - DNA, 37 bp, synthetic construct
  misc_feature 1..37: note = Description of Artificial Sequence: Synthetic oligonucleotide
  source 1..37: mol_type = other DNA
cttgatgtcagtctccttgatctcgaaccttcttcac

SEQ ID NO: 1415 - DNA, 39 bp, synthetic construct
  misc_feature 1..39: note = Description of Artificial Sequence: Synthetic oligonucleotide
  source 1..39: mol_type = other DNA
cttgatgtcagtctccttgaggtctcgaaccttcttcac

SEQ ID NO: 1416 - DNA, 39 bp, Mus musculus; identical to SEQ ID NO: 1411
  source 1..39: mol_type = other DNA

SEQ ID NO: 1417 - DNA, 37 bp, synthetic construct; identical to SEQ ID NO: 1414
  misc_feature 1..37: note = Description of Artificial Sequence: Synthetic oligonucleotide
  source 1..37: mol_type = other DNA

SEQ ID NO: 1418 - DNA, 39 bp, Mus musculus; identical to SEQ ID NO: 1411
  source 1..39: mol_type = other DNA

SEQ ID NO: 1419 - DNA, 39 bp, Mus musculus; identical to SEQ ID NO: 1411
  source 1..39: mol_type = other DNA

SEQ ID NO: 1420 - DNA, 39 bp, Mus musculus; identical to SEQ ID NO: 1411
  source 1..39: mol_type = other DNA

SEQ ID NO: 1421 - DNA, 37 bp, synthetic construct; identical to SEQ ID NO: 1414
  misc_feature 1..37: note = Description of Artificial Sequence: Synthetic oligonucleotide
  source 1..37: mol_type = other DNA

SEQ ID NO: 1422 - DNA, 37 bp, synthetic construct; identical to SEQ ID NO: 1414
  misc_feature 1..37: note = Description of Artificial Sequence: Synthetic oligonucleotide
  source 1..37: mol_type = other DNA

SEQ ID NO: 1423 - DNA, 39 bp, Mus musculus; identical to SEQ ID NO: 1411
  source 1..39: mol_type = other DNA

SEQ ID NO: 1424 - DNA, 39 bp, Mus musculus; identical to SEQ ID NO: 1411
  source 1..39: mol_type = other DNA

SEQ ID NO: 1425 - DNA, 39 bp, Mus musculus; identical to SEQ ID NO: 1411
  source 1..39: mol_type = other DNA

SEQ ID NO: 1426 - DNA, 39 bp, Mus musculus; identical to SEQ ID NO: 1411
  source 1..39: mol_type = other DNA

SEQ ID NO: 1427 - DNA, 37 bp, synthetic construct; identical to SEQ ID NO: 1414
  misc_feature 1..37: note = Description of Artificial Sequence: Synthetic oligonucleotide
  source 1..37: mol_type = other DNA

SEQ ID NO: 1428 - DNA, 39 bp, Mus musculus; identical to SEQ ID NO: 1411
  source 1..39: mol_type = other DNA

SEQ ID NO: 1429 - DNA, 38 bp, Homo sapiens
  source 1..38: mol_type = other DNA
gcacctggccccagttgtaattgtgaatatctcatacc

SEQ ID NO: 1430 - DNA, 43 bp, Homo sapiens
  source 1..43: mol_type = other DNA
gcacctggccccagttgtaattgtgaatatctcatacctatcc

SEQ ID NO: 1431 - DNA, 340 bp, Homo sapiens
  source 1..340: mol_type = other DNA
agtagagatggggtttcaccttgttagccaggatggtgtcgatctcctgaactcgtgatccacccgcctcggcctcctaaagtgctgggattacagatgtgagccaccgcacctggccccagttgtaattgtgaatatctcatacctatccctattggcagtgtcttagttttattttttattatctttattgtggcagccattattcctgtctctatctccagtcttacatcctccttactgccacaagaatgatcattctaaacatgaatcctaccctgtgactcccatgtgactccccgccttaaaaactgtcaaaagctaccggttacctgaaggg

SEQ ID NO: 1432 - DNA, 48 bp, Mus musculus
  source 1..48: mol_type = other DNA
  CDS 1..48: protein_id = 1462; transl_table = 1; translation = TGVVRSPFEQPQYYLA
acaggcgtggtgcggagccccttcgagcagccgcagtactacctggcg

SEQ ID NO: 1433 - protein, 16 aa, Mus musculus
TGVVRSPFEQPQYYLA

SEQ ID NO: 1434 - DNA, 48 bp, Mus musculus
  source 1..48: mol_type = other DNA
  CDS 1..48: protein_id = 1463; transl_table = 1; translation = TGVVRSPFEQPQYYLA
acaggcgtggtgcggagccctttcgagcagccgcagtactacctggcg

SEQ ID NO: 1435 - protein, 16 aa, Mus musculus
TGVVRSPFEQPQYYLA

SEQ ID NO: 1436 - DNA, 27 bp, Mus musculus
  source 1..27: mol_type = other DNA
  CDS 1..27: protein_id = 1464; transl_table = 1; translation = SPFEQPQYY
agccccttcgagcagccgcagtactac

SEQ ID NO: 1437 - protein, 9 aa, Mus musculus
SPFEQPQYY

SEQ ID NO: 1438 - DNA, 27 bp, Mus musculus
  source 1..27: mol_type = other DNA
  CDS 1..27: protein_id = 1465; transl_table = 1; translation = SHFEQPQYY
agccacttcgagcagccgcagtactac

SEQ ID NO: 1439 - protein, 9 aa, Mus musculus
SHFEQPQYY

SEQ ID NO: 1440 - DNA, 27 bp, Mus musculus
  source 1..27: mol_type = other DNA
  CDS 1..27: protein_id = 1466; transl_table = 1; translation = SPFEQPQYY
agccctttcgagcagccgcagtactac

SEQ ID NO: 1441 - protein, 9 aa, Mus musculus
SPFEQPQYY

SEQ ID NO: 1442 - DNA, 23 bp, Mus musculus
  source 1..23: mol_type = other DNA
gaaacagtttcgagatctcaggg

SEQ ID NO: 1443 - DNA, 23 bp, Mus musculus
  source 1..23: mol_type = other DNA
caagcaggttccagatctcaaag

SEQ ID NO: 1444 - DNA, 23 bp, Mus musculus
  source 1..23: mol_type = other DNA
gaagtagtttggagatctcatag

SEQ ID NO: 1445 - DNA, 23 bp, Mus musculus
  source 1..23: mol_type = other DNA
caggaaggttccagatctcacag

SEQ ID NO: 1446 - DNA, 23 bp, Mus musculus
  source 1..23: mol_type = other DNA
gagaaagttgcgagatctcagag

SEQ ID NO: 1447 - DNA, 23 bp, Mus musculus
  source 1..23: mol_type = other DNA
taagaaggatagagatctcaaag

SEQ ID NO: 1448 - DNA, 23 bp, Mus musculus
  source 1..23: mol_type = other DNA
agagaggtttcgagatctcagag

SEQ ID NO: 1449 - DNA, 23 bp, Mus musculus
  source 1..23: mol_type = other DNA
gattgagggtcgagatctcaaag

SEQ ID NO: 1450 - DNA, 23 bp, Mus musculus
  source 1..23: mol_type = other DNA
tagtaaggttggagatctcagag

SEQ ID NO: 1451 - DNA, 23 bp, Mus musculus
  source 1..23: mol_type = other DNA
acagaagattcaagatctcatgg

SEQ ID NO: 1452 - DNA, 23 bp, Mus musculus
  source 1..23: mol_type = other DNA
caggtaggttccagatctcaaag

SEQ ID NO: 1453 - DNA, 23 bp, Mus musculus
  source 1..23: mol_type = other DNA
gaagcaggtttaagatctcacag

SEQ ID NO: 1454 - DNA, 23 bp, Mus musculus
  source 1..23: mol_type = other DNA
ccagaagcttcgagatctcccag

SEQ ID NO: 1455 - DNA, 23 bp, Mus musculus
  source 1..23: mol_type = other DNA
atagaaggtcctagatctcacag

SEQ ID NO: 1456 - RNA, 93 nt, synthetic construct
  misc_feature 1..93: note = Description of Artificial Sequence: Synthetic oligonucleotide
  source 1..93: mol_type = other RNA
nnnnnnnnnnnnnnnnnnnngttttagagctagaaatagcttaaaataaggctaccccgttatcaacttgaagaattggcaccgagtcggtgc

SEQ ID NO: 1457 - skipped

SEQ ID NO: 1458 - protein, 45 aa, Mus musculus; identical to SEQ ID NO: 1406

SEQ ID NO: 1459 - protein, 5 aa, Mus musculus
KPHLE

SEQ ID NO: 1460 - protein, 77 aa, Mus musculus; identical to SEQ ID NO: 1407

SEQ ID NO: 1461 - protein, 30 aa, Mus musculus; identical to SEQ ID NO: 1409

SEQ ID NO: 1462 - protein, 16 aa, Mus musculus
TGVVRSPFEQPQYYLA

SEQ ID NO: 1463 - protein, 16 aa, Mus musculus
TGVVRSPFEQPQYYLA

SEQ ID NO: 1464 - protein, 9 aa, Mus musculus
SPFEQPQYY

SEQ ID NO: 1465 - protein, 9 aa, Mus musculus
SHFEQPQYY

SEQ ID NO: 1466 - protein, 9 aa, Mus musculus
SPFEQPQYY

(19) United States
(12) Patent Application Publication
(10) Pub. No.: US 2024/0336934 A1
(43) Pub. Date: Oct. 10, 2024

(54) CRISPR/CAS9-BASED COMPOSITIONS AND METHODS FOR TREATING RETINAL DEGENERATIONS

(71) Applicant: The Johns Hopkins University, Baltimore, MD (US)

(72) Inventors: Vinod Jaskula-Ranga, Cambridge, MA (US); Donald Zack, Baltimore, MD (US); Fred Bunz, Baltimore, MD (US); Derek Welsbie, Rancho Santa Fe, CA (US)

(21) Appl. No.: 18/380,920

(22) Filed: Oct. 17, 2023

Related U.S. Application Data
(63) Continuation of application No. 16/315,462, filed on Jan. 4, 2019, now abandoned.
(60) Provisional application No. 62/358,337, filed on Jul. 5, 2016.

(51) Int. Cl. C12N 15/86; A61K 38/46; A61K 48/00; C12N 9/22; C12N 15/113; C12N 15/90

(52) U.S. Cl. CPC C12N 15/86; A61K 38/465; A61K 48/005; A61K 48/0075; C12N 9/22; C12N 15/113; C12N 15/907; C12N 2310/121; C12N 2310/20; C12N 2310/3519; C12N 2320/35; C12N 2330/51; C12N 2750/14343; C12N 2830/205

(57) ABSTRACT

Described herein are methods for treating a retinal degeneration in a subject, such as Leber's congenital amaurosis (LCA), retinitis pigmentosa (RP), and glaucoma. Also provided herein are methods of altering expression of one or more gene products in a cell, such as a retinal ganglion cell. Such methods may comprise utilizing a modified nuclease system, such as Clustered Regularly Interspaced Short Palindromic Repeats (CRISPR) system comprising a bidirectional HI promoter and gRNAs directed to retinal degeneration related genes, packaged in a single, compact adeno-associated virus (AAV) particle.

### CROSS-REFERENCE

This application claims the benefit of U.S. Provisional Application No. 62/358,337, filed Jul. 5, 2016, the entirety of which is hereby incorporated by reference.

### BACKGROUND

Retinal degenerations are a group of disorders which include Leber's congenital amaurosis (LCA), retinitis pigmentosa (RP), and glaucoma, among others. LCA is a heritable form of retinal degeneration characterized by severe retinal dysfunction and severe visual impairment during the first months of life. LCA is an orphan disease (one that affects fewer than 200,000 Americans), but the 18 subtypes of LCA are together the most common cause of inherited blindness. The subtype designated LCA10, which is the most common subtype, accounting for >20% of all LCA cases. Some forms of LCA are amenable to treatment by recombinant adeno-associated viruses (AAVs) engineered to deliver a functional copy of the defective cellular gene. In 2008, a transgene that complemented the mutation in RPE65 was successfully delivered by AAV to LCA2 patients in a Phase I Clinical trial (Maguire A M et al. N Engl J Med. 2008; 358(21): 2240-2248). Some responses were noted, but these were not durable because transgene expression was eventually lost (Schimmer J et al. Hum Gene Ther Clin Dev. 2015; 26(4): 208-210; Azvolinsky A. Nat Biotechnol. 2015; 33(7): 678-678). Furthermore, some of the genes that cause the different LCA subtypes are simply too large for AAV delivery. These subtypes of LCA therefore remain untreatable.

The ADRP constitutes approximately 30-40% of all cases of RP, and among ADRP patients the most commonly mutated RP associated gene is the one that encodes the rod visual pigment rhodopsin (Dryja, T. P. et al. The New England journal of medicine 323, 1302-1307 (1990); Dryja, T. P. et al. Nature 343, 364-366 (1990)). At the moment, there are no FDA approved treatments for ADRP patients; however, a number of approaches are being developed. Most of these approaches are variations on the theme of “suppression and replacement.” In this approach, one knocks down expression of the gene responsible for degeneration, for example knocking down levels of rhodopsin RNA with a ribozyme or via RNA interference (RNAi) (both shRNAs and siRNA methodologies are being explored), and then replaces expression of the endogenous alleles with a “hardened” gene that is not susceptible to knock down by the ribozyme or RNAi agent. The variant of this theme that is perhaps closest to the clinic is the RhoNova agent being developed by Genable Technologies Limited. RhoNova employs an siRNA to knock down endogenous rhodopsin expression (both mutant and wild-type) combined with an AAV-delivered cDNA that encodes a modified but functional rhodopsin that is not susceptible to siRNA knock down (http://www.genable.net).

Glaucoma, the leading cause of irreversible blindness worldwide (Levkovitch-Verbin H et al. iovsorg 44, 3388-3393 (2003)), is an optic neuropathy in which progressive damage of retinal ganglion cell (RGC) axons at the lamina cribosa of the optic nerve head leads to axon degeneration and cell death (Howell G R et al. J Cell Biol 179, 1523-1537 (2007)). Currently, the only treatment, whether by eye drops, lasers or incisional surgery, is to lower intraocular pressure (IOP) and reduce the injury at the optic nerve head. Unfortunately, this is difficult in some patients while in others, the disease can continue to worsens despite aggressive IOP-lowering. The field has long needed an alternative therapeutic strategy that could complement IOP-lowering by mitigating the RGC response to residual axon injury. Moreover, the NEI has listed optic nerve regeneration amongst its Audacious Goals, and any regenerative therapy necessarily needs to tackle the issue of axotomized RGC survival. To this end, there is a great need to develop a neuroprotective that might directly interfere with the active genetic programs of RGC axon degeneration and/or axon injury-related cell death (Adalbert R et al. Science (2012), doi:10.1126/science.1223899; Yang J et al. Cell 160, 161-176 (2015); Welsbie D S et al. Proc Nat Acad Sci USA 110, 4045-4050 (2013); Watkins T A et al. Proc Nat Acad Sci USA 110, 4039-4044 (2013)).

Thus there is a great need for novel and improved therapies for treating retinal degenerations, like LCA, ADRP, and glaucoma.

### SUMMARY

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning. A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange 10^{th}ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, available on the World Wide Web: http://www.ncbi.nlm.nih.gov/omim/ and in Online Mendelian Inheritance in Animals (OMIA), a database of genes, inherited disorders and traits in animal species (other than human and mouse), available on the World Wide Web: http://omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

Described herein are methods for treating retinal degenerations, such as optic neuropathies including Leper's congenital amaurosis (e.g., Leber's congenital amaurosis 10 CEP290 mutation (LCA)), retinitis pigmentosa (e.g., Rhodopsin R135 mutations), or glaucoma. The methods use a modified nuclease system, such as Clustered Regularly Interspaced Short Palindromic Repeats (CRISPR) (e.g. CRISPR associated (Cas) 9 (CRISPR-Cas9, non-Cas9 CRISPR systems, CRISPR-Cpf-1 system, and the like), to cut and/or repair genomic DNA or RNA (e.g., Cas13a/C2c2 system). The CRISPR-system-based gene editing can be used to inactivate or correct gene mutations causing optic neuropathies and retinal degenerations (e.g., LCA and rhodopsin mutations), thereby providing a gene therapy approach for these groups of diseases. In some embodiments, the CRISPR system is used to introduce a mutation that will inactivate a normal gene (e.g. DLK and/or LZK) causing retinal degeneration (e.g. glaucoma). Because these genes play roles in damage-sensing and cell-survival, the resulting effect is cell survival. The “neuroprotective” approach is not a mutually exclusive approach as there are genetic mutations that could lead to glaucoma as well, and these would be the same as the retinal degenerations. In some embodiments, the mutation targets of glaucoma include, but not limited to, OPTN, TBK1, TMCO1, PMM2, GMDS, GAS7, FNDC3B, TXNRD2, ATXN2, CAV1/CAV2, p16INK4a, SIX6, ABCA1, AFAP1 and CDKN2B-AS.

Thus, one aspect of the invention relates to a method for treating a disorder (e.g., retinal degenerations) affecting a retina area of a subject, the method comprising administering to the retina area of the subject a therapeutically effective amount of a nuclease system comprising a genome targeted nuclease and a guide DNA comprising at least one targeted genomic sequence.

Another aspect of the invention provides methods for treating retinal degenerations utilize a composition comprising a modification of a non-naturally occurring CRISPR system previously described in WO2015/195621 (herein incorporated by reference in its entirety). Such a modification uses certain gRNAs that target retinal degeneration-related genes, such as, but not limited, to LCA10 CEP290 gene, rhodopsin, Dual Leucine Zipper Kinase (DLK), Leucine Zipper Kinase (LZK), JNK1-3, MKK4, MKK7, ATF2, JUN, MEF2A, SOX11, or PUMA. In some embodiments, the composition comprises (a) a non-naturally occurring nuclease system (e.g., CRISPR) comprising one or more vectors comprising: i) a promoter (e.g., bidirectional H1 promoter) operably linked to at least one nucleotide sequence encoding a nuclease system guide RNA (gRNA), wherein the gRNA hybridizes with a target sequence of a DNA molecule in a cell of the subject, and wherein the DNA molecule encodes one or more gene products expressed in the cell; and ii) a regulatory element operable in a cell operably linked to a nucleotide sequence encoding a genome-targeted nuclease (e.g., Cas9 protein), wherein components (i) and (ii) are located on the same or different vectors of the system, wherein the gRNA targets and hybridizes with the target sequence and the nuclease cleaves the DNA molecule to alter expression of the one or more gene products. In some embodiments, the system is packaged into a single adeno-associated virus (AAV) particle. In some embodiments, the promoter comprises: a) control elements that provide for transcription in one direction of at least one nucleotide sequence encoding a gRNA; and b) control elements that provide for transcription in the opposite direction of a nucleotide sequence encoding a genome-targeted nuclease.

Another aspect of the invention provides methods of altering expression of one or more gene products in a eukaryotic cell, wherein the cell comprises a DNA molecule encoding the one or more gene products, the method comprising introducing into the cell a modified non-naturally occurring CRISPR system previously described in WO2015/195621 (herein incorporated by reference in its entirety). Such a modification uses certain gRNAs that target retinal degeneration-related genes, such as, but not limited, to LCA10 CEP290 gene, rhodopsin, Dual Leucine Zipper Kinase (DLK), Leucine Zipper Kinase (LZK), JNK1-3, MKK4, MKK7, ATF2, JUN, MEF2A, SOX11, or PUMA. In some embodiments, the method comprising introducing into the cell a composition comprising (a) a non-naturally occurring nuclease system (e.g., CRISPR) comprising one or more vectors comprising: i) a promoter (e.g., bidirectional H1 promoter) operably linked to at least one nucleotide sequence encoding a nuclease system guide RNA (gRNA), wherein the gRNA hybridizes with a target sequence of a DNA molecule in a cell of the subject, and wherein the DNA molecule encodes one or more gene products expressed in the cell; and ii) a regulatory element operable in a cell operably linked to a nucleotide sequence encoding a genome-targeted nuclease (e.g., Cas9 protein), wherein components (i) and (ii) are located on the same or different vectors of the system, wherein the gRNA targets and hybridizes with the target sequence and the nuclease cleaves the DNA molecule to alter expression of the one or more gene products. In some embodiments, the system is packaged into a single adeno-associated virus (AAV) particle. In some embodiments, the promoter comprises: a) control elements that provide for transcription in one direction of at least one nucleotide sequence encoding a gRNA; and b) control elements that provide for transcription in the opposite direction of a nucleotide sequence encoding a genome-targeted nuclease.

One aspect of the invention, relates to a method for treating a retinal degeneration in a subject in need thereof, the method comprising: (a) providing a non-naturally occurring nuclease system comprising one or more vectors comprising: i) a promoter operably linked to at least one nucleotide sequence encoding a nuclease system guide RNA (gRNA), wherein the gRNA hybridizes with a target sequence of a DNA molecule in a cell of the subject, and wherein the DNA molecule encodes one or more gene products expressed in the cell; and ii) a regulatory element operable in a cell operably linked to a nucleotide sequence encoding a genome-targeted nuclease, wherein components (i) and (ii) are located on the same or different vectors of the system, wherein the gRNA targets and hybridizes with the target sequence and the nuclease cleaves the DNA molecule to alter expression of the one or more gene products; and (b) administering to the retinal area of the subject a therapeutically effective amount of the system.

In some embodiments, the system is CRISPR.

In some embodiments, the system is packaged into a single adeno-associated virus (AAV) particle.

In some embodiments, the system inactivates one or more gene products.

In some embodiments, the nuclease system excises at least one gene mutation.

In some embodiments, the promoter comprises a bidirectional promoter. In some embodiments, the promoter comprises a nucleotide sequence having at least 80%, 85%, 90%, 95%, 98%, 99%, or 100% identity to the nucleotide sequence selected from the group consisting of SEQ ID NOs: 739-787. In some embodiments, the promoter comprises a nucleotide sequence selected from the group consisting of SEQ ID NOs: 739-787.

In some embodiments, the bidirectional promoter is H1 (SEQ ID NO: 787). The H1 promoter is both a pol II and pol III promoter. In some embodiments, the promoter is orthologous to the H1 promoter.

In some embodiments, the orthologous H1 promoter is derived from eutherian mammals.

In some embodiments, the orthologous H1 promoter is derived from Ailuropoda melanoleuca, Bos taurus, Callithrix jacchus, Canis familiaris, Cavia porcellus, Chlorocebus sabaeus, Choloepus hofmanni, Dasypus novemcinctus, Dipodomys ordii, Equus caballus, Erinaceus europaeus, Felis catus, Gorilla gorilla, Homo sapiens, Ictidomys tridecemlineatus, Loxodonta africana, Macaca mulatta, Mus musculus, Mustela putorius furo, Myotis lucifugus, Nomascus leucogenys, Ochotona princeps, Oryctolagus cuniculus, Otolemur garnettii, Ovis aries, Pan troglodytes, Papio anubis, Pongo abelii, Procavia capensis, Pteropus vampyrus, Rattus norvegicus, Sus scrofa, Tarsius syrichta, Tupaia belangeri, Tursiops truncatus, Vicugna pacos.

In some embodiments, the orthologous H1 promoter is derived from mouse or rat.

In some embodiments, the orthologous H1 promoter comprises a nucleotide sequence having at least 80%, 85%, 90%, 95%, 98%, 99%, or 100% identity to the nucleotide sequence set forth in SEQ ID NOs: 752-786.

In some embodiments, the orthologous H1 promoter comprises a nucleotide sequences set forth in the group consisting of SEQ ID NOs: 752-786.

In some embodiments, the H1 promoter comprises: a) control elements that provide for transcription in one direction of at least one nucleotide sequence encoding a gRNA; and b) control elements that provide for transcription in the opposite direction of a nucleotide sequence encoding a genome-targeted nuclease.

In some embodiments, the genome-targeted nuclease is Cas9 protein.

In some embodiments, the Cas9 protein is codon optimized for expression in the cell.

In some embodiments, the promoter is operably linked to at least one, two, three, four, five, six, seven, eight, nine, or ten gRNA.

In some embodiments, the retinal area is the retina.

In some embodiments, the cell is a retinal photoreceptor cell.

In some embodiments, the cell is a retinal ganglion cell.

In some embodiments, the retinal degeneration is selected from the group consisting of Leber's congenital amaurosis (LCA), retinitis pigmentosa (RP), and glaucoma.

In some embodiments, the retinal degeneration is LCA1, 2, 3, 4, 5, 6, 7, 8, 9, 10, 11, 12, 13, 14, 15, 16, 17, or 18.

In some embodiments, the retinal degeneration is LCA10.

In some embodiments, the target sequence is in the LCA10 CEP290 gene.

In some embodiments, the target sequence is a mutation in the CEP290 gene.

In some embodiments, the target sequence is selected from the group consisting of the nucleotide sequences set forth in SEQ ID NO: 1-109, 164-356, 735-738, or combinations thereof.

In some embodiments, the target sequence comprises SEQ ID NOs: 1, 2, 3, and 4 operably linked.

In some embodiments, the vector comprises the nucleotide sequence set forth in SEQ ID NO: 110.

In some embodiments, the retinal degeneration is an autosomal dominant form of retinitis pigmentosa (ADRP).

In some embodiments, the one or more gene products are rhodopsin.

In some embodiments, the target sequence is a mutation in the rhodopsin gene.

In some embodiments, the target sequence is a mutation at R135 of the rhodopsin gene.

In some embodiments, the mutation at R135 is selected from the group consisting of R135G, R135W, R135L.

In some embodiments, the target sequence is selected from the group consisting of the nucleotide sequences set forth in SEQ ID NO: 111-126, or combinations thereof.

In some embodiments, the gRNA sequence is selected from the group consisting of the nucleotide sequences set forth in SEQ ID NO: 127-142, or combinations thereof.

In some embodiments, the retinal degeneration is glaucoma.

In some embodiments, the one or more gene products are Dual Leucine Zipper Kinase (DLK), Leucine Zipper Kinase (LZK), ATF2, JUN, sex determining region Y (SRY)-box 11 (SOX11), myocyte enhancer factor 2A (MEF2A), JNK1-3, MKK4, MKK7, SOX11, or PUMA, or combinations thereof.

In some embodiments, the one or more gene product are members of the DLK/LZK, MKK4/7, JNK1/2/3 or SOX11/ATF2/JUN/MEF2A pathway.

In some embodiments, the target sequence is selected from the group consisting of the nucleotide sequences set forth in SEQ ID NO: 143-163, or combinations thereof.

In some embodiments, administering to the subject occurs by implantation, injection, or virally.

In some embodiments, administering to the subject occurs by subretinal injection.

In some embodiments, the subject is human.

Another aspect of the invention relates to a method of altering expression of one or more gene products in a cell, wherein the cell comprises a DNA molecule encoding the one or more gene products, the method comprising introducing into the cell a non-naturally occurring nuclease system comprising one or more vectors comprising: a) a promoter operably linked to at least one nucleotide sequence encoding a nuclease system guide RNA (gRNA), wherein the gRNA hybridizes with a target sequence of the DNA molecule; and
b) a regulatory element operable in the cell operably linked to a nucleotide sequence encoding a genome-targeted nuclease,
   wherein components (a) and (b) are located on the same or different vectors of the system, wherein the gRNA targets and hybridizes with the target sequence and the nuclease cleaves the DNA molecule to alter expression of the one or more gene products.

In some embodiments, the system is CRISPR.

In some embodiments, the system is packaged into a single adeno-associated virus (AAV) particle.

In some embodiments, the system inactivates one or more gene products.

In some embodiments, the nuclease system excises at least one gene mutation.

In some embodiments, the promoter is a bidirectional promoter.

In some embodiments, the bidirectional promoter is H1. The H1 promoter is both a pol II and pol III promoter.

In some embodiments, the H1 promoter comprises: a) control elements that provide for transcription in one direction of at least one nucleotide sequence encoding a gRNA; and b) control elements that provide for transcription in the opposite direction of a nucleotide sequence encoding a genome-targeted nuclease.

In some embodiments, the genome-targeted nuclease is Cas9.

In some embodiments, the Cas9 protein is codon optimized for expression in the cell.

In some embodiments, the promoter is operably linked to at least one, two, three, four, five, six, seven, eight, nine, or ten gRNA.

In some embodiments, the cell is a eukaryotic or non-eukaryotic cell.

In some embodiments, the eukaryotic cell is a mammalian or human cell.

In some embodiments, the cell is a retinal photoreceptor cell.

In some embodiments, the cell is a retinal ganglion cell.

In some embodiments, the one or more gene products are LCA10 CEP290.

In some embodiments, the target sequence is selected from the group consisting of the nucleotide sequences set forth in SEQ ID NO: 1-109, 164-356, 735-738, or combinations thereof.

In some embodiments, the target sequence comprises SEQ ID NOs: 1, 2, 3, and 4 operably linked.

In some embodiments, the vector comprises the nucleotide sequence set forth in SEQ ID NO: 110.

In some embodiments, the one or more gene products are rhodopsin.

In some embodiments, the target sequence is a mutation in the rhodopsin gene.

In some embodiments, the target sequence is a mutation at R135 of the rhodopsin gene.

In some embodiments, the mutation at R135 is selected from the group consisting of R135G, R135W, R135L.

In some embodiments, the target sequence is selected from the group consisting of the nucleotide sequences set forth in SEQ ID NO: 111-126, or combinations thereof.

In some embodiments, the gRNA sequence is selected from the group consisting of the nucleotide sequences set forth in SEQ ID NO: 127-142, or combinations thereof.

In some embodiments, the one or more gene products are Dual Leucine Zipper Kinase (DLK), Leucine Zipper Kinase (LZK), ATF2, JUN, sex determining region Y (SRY)-box 11 (SOX11), myocyte enhancer factor 2A (MEF2A), JNK1-3, MKK4, MKK7, SOX11, or PUMA, or combinations thereof.

In some embodiments, the one or more gene product are members of the DLK/LZK, MKK4/7, JNK1/2/3 or SOX11/ATF2/JUN/MEF2A pathway.

In some embodiments, the target sequence is selected from the group consisting of the nucleotide sequences set forth in SEQ ID NO: 143-163, or combinations thereof.

In some embodiments, the expression of the one or more gene products is decreased.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 shows the platform technology. The genomic sequence of the H1 bidirectional promoter with a pol II transcript shown in blue and a pol III transcript shown in orange (left). Packaging of the spCas9 and a gRNA within a single AAV vector (right).

FIG. 2 shows delivery of AAV-H1-CRISPR to the mouse retina. Virus engineered to express GFP in place of Cas9 demonstrates both efficient and specific transduction of mouse photoreceptors in the outer nuclear layer (ONL) after sub-retinal injection. These are the cells that are affected by LCA mutations.

FIG. 3 shows an illustration of the Cas9 nickase approach, which require two closely opposed target sites (L and R) on opposite strands.

FIG. 4 shows the LCA10 mutation. The four identified gRNA sites that would result in ˜1 kb deletion, removing the cryptic Exon X from CEP290.

FIG. 5 shows the current SaCas9 approach delivering 2 gRNAs at 4550 bp (left) versus compact H1 system delivering 4 gRNAs using 4335 bp (right). The AAV packaging capacity is indicated by the dotted line.

FIG. 6 shows all SaCas9 sites (1 kb upstream and 1 kb downstream of the CEP290 mutation).

FIG. 7 shows the SaCas9 sites available for targeting (All start with 5′G).

FIG. 8 shows a much safer SaCas9 nickase approach.

FIG. 9 shows a SaCas9 nickase deletion (1078 bp).

FIG. 10 shows potential SpCas9 sites.

FIG. 11 shows the number of CRISPR sites for SaCas9 or SpCas9 by 5′ nucleotide in the CEP290 (LCA10) targeting region.

FIG. 12 shows cloned ˜4.2 kb SaCas9 construct with four gRNAs.

FIG. 13 shows the rhodopsin gene structure.

FIG. 14 shows mutation spectrum of the RHO gene worldwide (from http://www.hindawi.com/journals/bmri/2014/302487/)

FIG. 15 shows the rhodopsin Arg135 mutation.

FIG. 16 shows the R135W Pedigree of 2 French Families (from Audo I et al. Invest Ophthalmol Vis Sci. (2010) July; 51(7):3687-700).

FIG. 17 shows the R135W from 5-generation Sicilian Pedigree (from Pannarale M R et al. Ophthalmology. (1996) September; 103(9):1443-52).

FIG. 18 shows the Six generation Swedish Family with R135L (from Andréasson S et al. Ophthalmic Paediatr Genet. (1992) September; 13(3):145-53).

FIG. 19 shows sensitized kinome screen identifies LZK as cooperating with DLK to promote RGC cell death.

FIG. 20 shows whole genome siRNA screens identify ATF2, SOX11, and MEF2A as mediators of RGC cell death.

FIG. 21 shows downstream mediators of LZK/DLK-dependent RGC cell death.

FIG. 22 shows calcium-sensing motif in LZK is dispensable for toxicity.

FIG. 23 shows hammerhead ribozyme-sgRNA fusions to increase the number of targetable spCas9 sites.

FIG. 24 shows network-based siRNA and siPOOL screening has improved sensitivity and specificity.

FIG. 25 shows H1 promoter allows for bidirectional expression of Pol II and Pol III transcripts.

FIG. 26 shows flow cytometry-based quantification of RGCs.

FIG. 27 shows CRISPR targeting of DLK exon 1 (A) and exon 2 (B) in vitro. Exon 1 and Exon 2 of DLK with target sites shown in blue, and T7E1 primers shown in green.

FIG. 28 comprises two panels, A and B, showing CRISPR targeting of DLK in vitro. FIG. 28A shows screening gRNAs for their ability to target the DLK gene from mouse. Target site nomenclature is according to http://crispr.technology. FIG. 28B shows in vitro cleavage using a bidirectional promoter to express Cas9 and a gRNA demonstrates efficient targeting of the DLK locus. The control is a standard 2-plasmid transfection for Cas9 and a gRNA. FIG. 28B shows experiments testing the ability to drive both Cas9 and a gRNA from the H1 bidirectional promoter. Cells in culture were transfected with either the standard two plasmids (Cas9 and gRNA) or a single plasmid using the H1 bidirectional promoter. T7EI assay indicates comparable levels of cutting using either system.

FIG. 29 shows DLK targeting by AAV in vitro.

FIG. 30 comprises three panels, A, B, and C, showing bidirectional expression in RGCs in vivo. FIG. 30A shows construct that was packaged into AAV. FIG. 30B shows the cell-type expression of GFP in vivo is affected by the AAV serotype used. Top shows preferential expression in photoreceptors and the lower panel shows preferential expression in RGCs. The H1 promoter clearly expresses GFP in either photoreceptor cells delivered by AAV5, or in retinal ganglion cells by AAV2 (control). Both viruses were delivered by subretinal injection to P0.5 day mice. Both were delivered by sub-retinal injection of the reporter indicated in FIG. 30A. FIG. 30C shows GFP expression by flatmount following 15 days of AAV2 intravitreal delivery of the reporter construct.

FIG. 31 comprises three panels, A, B, and C, showing bidirectional targeting using self-complementary AAV viruses. FIG. 31A shows self-complementary AAV construct that expresses a nuclear mCherry and a gRNA from a bidirectional promoter. This figure further shows experiments testing the ability to use self-complementary AAV (scAAV) to delivery a gRNA and a fluorescent reporter protein (H2B-mCherry). Cells were harvested from the Cas9 mouse, and transduced in vitro. The benefits of scAAV is the ability to test constructs much faster as expression occurs in days and not weeks. FIG. 31A shows a construct was generated using the H1 promoter to express a gRNA (shown in black) and mCherry simultaneously. The gRNA targets the DLK mouse gene, a gene that when inactivated, results in enhanced retinal ganglion cell survival. The construct was packaged to produce a self-complementary AAV (scAAV). Retinal ganglion cells were harvested from the Cas9 transgenic mouse (which co-expresses GFP), and transduced with the scAAV virus; mCherry expression was apparent in all cells expressing GFP, indicating highly efficient transduction and expression from the construct. FIG. 31A also shows testing the ability to use self-complementary AAV (scAAV) to delivery a gRNA and a fluorescent reporter protein (H2B-mCherry). Cells were harvested from the Cas9 mouse, and transduced in vitro. The benefits of scAAV is the ability to test constructs much faster as expression occurs in days and not weeks. FIG. 31B shows in vitro expression of the scAAV reporter transducing RGCs in vitro; GFP expression is from the Cas9 mouse. FIG. 31C shows highly-efficient targeting (essentially) 100% as detected by a BglII assay. The gRNA (mm079) was delivered by ssAAV and Cas9 was present from the mouse

FIG. 32 comprises five panels, A-E, showing bidirectional targeting using self-complementary AAV viruses. Titration of scAAV virus transducing either WT RGC or RGCs derived from the Cas9 mouse. FIG. 32C shows that genome-editing occurs in RGCs when Cas9 is present. These in vitro experiments demonstrate very high levels (˜100%) cutting. In this assay, we are assaying for cutting by loss of a restriction enzyme site. In the WT animals, the PCR product is fully digested by BglII, however, in Cas9 mice transduced with AAV, there is essentially undetectable BglII cutting at the highest concentration, indicating ˜100% CRISPR cutting. FIGS. 32D and 32E show in vivo rescue of retinal ganglion cells following optic nerve crush in treated eyes. A construct was generated using the H1 promoter to express a gRNA (shown in black) and mCherry simultaneously. The gRNA targets the DLK mouse gene, a gene that when inactivated, results in enhanced retinal ganglion cell survival. The construct was packaged to produce a self-complementary AAV (scAAV). The virus was administered intravitreally into the Cas9 transgenic, or a WT mouse as control. Retinal ganglion cell survival was quantitated 14 days following optic nerve crush, indicating that CRISPR delivery resulted in RGC survival in the treated mouse, as compared to the control. (Both mice receive the CRISPR gRNA, but the difference between the mice is the presence of Cas9, which is required for genome-editing.) FIG. 33 comprises three panels, A, B, and C, showing CRISPR targeting of DLK results in RGC survival. Transduction of RGCs from the Cas9 mouse by lentivirus results in ˜100% cutting as measured by BglII assay. Disruption of DLK results in a significant increase in RGC survival, demonstrating the potential for DLK targeting as therapeutical target in optic neuropathies.

FIG. 34 comprises two panels, A and B, showing CRISPR targeting of LZK in vitro. FIG. 34A shows exon 1 of LZK with target sites shown in blue, and T7E1 primers shown in green. FIG. 34B shows exon 2 of LZK with target sites shown in blue, and T7E1 primers shown in green.

FIG. 35 comprises seven panels, A-H, showing sensitized siRNA screening of the kinome identifies LZK as a mediator of RGC cell death in vitro. FIG. 35A shows survival of Dlk^{fl/fl} RGCs transduced with Cre-expressing or control adenovirus and cultured in the presence of tozasertib (1 μM) or a vehicle control. FIG. 35B depicts a histogram showing the normalized survival for all 1,869 siRNAs in the kinome library (transfected in the presence of Dlk siRNA). Arrows show the survival for each of the three siRNAs for Lzk. FIG. 35C shows survival of WT RGCs transfected with control or Dlk siRNA, in combination with one of four independent Lzk siRNAs or the nontargeting control. FIG. 35D shows capillary-based immunoassay of WT RGCs after transfection with control, Dlk, Lzk or both Dlk and Lzk siPOOLs. FIG. 35E shows survival of WT RGCs transfected with increasing amounts control, Dlk, Lzk or both Dlk and Lzk siPOOLs. FIG. 35F shows survival of WT RGCs transfected with Dlk siRNA and either control siRNAs or one of four independent siRNAs targeting the other members of the mixed-lineage kinase family of kinases. FIG. 35G shows survival of WT RGCs transfected with control, Dlk, Lzk or both Dlk and Lzk siPOOLs and cultured in the presence of increasing doses of tozasertib. FIG. 35H depicts Survival (±SD) of WT RGCs transfected with siPOOLs, in the presence or absence of neurotrophins (NTs, 50 ng/mL BDNF, 5 ng/mL GDNF, 5 ng/mL CNTF), two days after colchicine (1 μM) addition. *P<0.05, Mann-Whitney U test. D/L, Dlk/Lzk.

FIG. 36 comprises six panels, A and F, showing RGCs with a targeted deletion of Dlk and Lzk are highly resistant to axon injury-induced cell death in vitro and in vivo. FIG. 36A shows a diagram of the approach used to generate constitutive and conditional Lzk knockout mice. Inset shows a Southern blot confirming the presence of a single targeting construct in the heterozygous animals. FIG. 36B shows a capillary-based immunoassay (top) and quantification (bottom) of RGCs isolated from WT vs. Lzk^{-/-} mice, 0 or 24 hours after the immunopanning injury. FIG. 36C shows a flow cytometry-based quantification of the number of surviving RGCs, normalized to the uninjured control, two weeks after optic nerve crush or a sham control. NS, nonsignificant, Mann-Whitney U test. FIG. 36D shows a capillary-based immunoassay (top) and quantification (bottom) of RGCs isolated from WT or Lzk^{fl/fl} mice and transduced with Cre-expressing or control adenovirus. FIG. 36E shows survival WT, Dlk^{fl/fl}, Lzk^{fl/fl} or Dlk^{fl/fl}Lzk^{fl/fl} RGCs, transduced with increasing amounts of either Cre-expressing or control adenovirus. FIG. 36F shows flow cytometry-based quantification of the number of surviving RGCs, normalized to the uninjured control, two weeks after optic nerve crush or a sham control. All eyes were injected with 10⁹ vg AAV2-Cre two weeks prior to the surgery. *P<0.05, Mann-Whitney U test.

FIG. 37 comprises five panels, A-E, showing LZK kinase signaling triggers RGC cell death via the MKK4/7 and JNK1-3 kinase cascade. FIG. 37A shows survival of WT RGCs transfected with Dlk/Lzk siPOOL and then reconstituted with LZK signaling by transducing with adenovirus expressing WT or mutant, siPOOL-resistant, human LZK cDNA. FIG. 37B-C show survival of WT (B-C), Jnk1^{fl/fl}Jnk2−/−Jnk3^{-/-} (B) or Mkk4^{fl/fl}Mkk7^{fl/fl} (C) RGCs transduced with increasing amounts of Cre-expressing or control adenovirus. FIG. 37D-E show survival of WT (D-E), Jnk1^{fl/fl}Jnk2−/−Jnk3^{-/-} (D) or Mkk4^{fl/fl}Mkk7^{fl/fl} (E) RGCs transfected with Dlk/Lzk siPOOL, transduced with Cre-expressing or control adenovirus, and then, two days later, with reconstitution of LZK signaling by transducing with human LZK cDNA-expressing or control adenovirus.

FIG. 38 comprises three panels, A-C, showing whole-genome siRNA screen identifies ATF2, PUMA and MEF2A as mediators of RGC cell death. FIG. 38A depicts a histogram showing the normalized, seed-adjusted survival for the median survival-promoting siRNA targeting each of the 17,575 genes in the whole-genome library. Arrows show the survival for the median survival-promoting siRNAs targeting Atf2, Puma and Mef2a. FIG. 38B depicts survival of WT RGCs transfected with one of four independent siRNAs targeting Atf2, Puma or Mef2a or the nontargeting control. Dashed line shows the threshold of survival greater than 3SD from the negative control. FIG. 38C shows survival of WT RGCs transfected with increasing amounts of control and either Atf2 (left), Puma (middle) or Mef2a (right) siPOOL.

FIG. 39 comprises seven panels, A-G, showing RGCs with a targeted disruption of the transcriptional regulatory domains of ATF2 and MEF2A are partially resistant to axon injury-induced cell death in vivo. FIG. 39A shows survival of WT RGCs transfected with Dlk/Lzk or Puma siRNA and transduced with WT or KD human LZK. FIG. 39B shows capillary-based immunoassay of Mef2a^{fl/fl} RGCs transduced with Cre-expressing or control adenovirus. FIG. 39C shows survival of WT or Mef2a^{fl/fl} RGCs transduced with increasing amounts of Cre-expressing or control adenovirus. FIG. 39D shows fold-change in survival with the transduction of Mef2a^{fl/fl} versus Mef2a^{fl/fl}Mef2c^{fl/fl}Mef2d^{fl/fl} RGCs with Cre-expressing or control adenovirus. FIG. 39E shows flow cytometry-based quantification of the number of surviving RGCs, normalized to the uninjured control, two weeks after optic nerve crush or a sham control. All eyes were injected with 10⁹ vg AAV2-Cre two weeks prior to the surgery. *P<0.05, Mann-Whitney U test. FIG. 39F shows flow cytometry-based quantification of the number of TUBB3/P-S408 MEF2A, expressed as a percentage of total, two days after an optic nerve crush or the sham control. FIG. 39G shows survival of WT or Atf2^{fl/fl} RGCs transduced with increasing amounts of Cre-expressing or control adenovirus.

FIG. 40 comprises six panels, A-G, showing sensitized whole-genome siRNA screen identifies JUN and SOX11 as mediators of RGC cell death. FIG. 40A depicts a histogram showing the normalized survival for the siRNA minipool targeting each of the genes in the whole-genome library (transfected in the presence of Lzk siPOOL). Arrow shows the survival for the top siRNA minipool, targeting Dlk. FIG. 40B shows quantitative PCR (qPCR) assay for Sox11 mRNA, normalized to GAPDH levels, in WT RGCs at the indicated time following immunopanning injury and transfected with either control siPOOLs or siPOOLs against Dlk/Lzk or Sox11. FIG. 40C shows survival of WT RGCs transfected with increasing amounts of Lzk and/or Sox11 siPOOL. FIG. 40D shows survival of WT RGCs transfected with Lzk or Lzk/Sox11 siPOOLs and reconstituted for SOX11 signaling by transducing with control or human SOX11 cDNA-expressing adenovirus. FIG. 40E shows survival of WT or Sox11^{fl/fl} RGCs transduced with increasing amounts of Cre-expressing or control adenovirus. FIG. 40F shows flow cytometry-based quantification of the number of surviving RGCs, normalized to the uninjured control, two weeks after optic nerve crush or a sham control. All eyes were injected with 10⁹ vg AAV2-Cre two weeks prior to the surgery. *P<0.05, Mann-Whitney U test. FIG. 40G depicts QPCR assay of Sox11 mRNA, normalized to GAPDH levels, in Sox11fl/fl RGCs transduced with adenovirus.

FIG. 41 comprises seven panels, A-H, showing DLK/LZK-dependent cell death is mediated by a set of four transcription factors: JUN, ATF2, SOX11 and MEF2A. FIG. 41A shows survival of WT RGCs transfected with the indicated siPOOLs. FIG. 41B shows survival of WT RGCs transfected with Dlk/Lzk siPOOLs and either control or Jun/Atf2/Sox11/Mef2a siPOOLs, and then reconstituted with LZK signaling by transducing with siPOOL-resistant, human LZK cDNA-expressing or control adenovirus. FIG. 41C shows survival of WT or SpCas9 knockin RGCs transfected with Lzk siPOOL and either tracrRNA or sgRNAs targeting Dlk. FIG. 41D shows survival of WT or SpCas9 knockin RGCs transfected with Dlk siPOOL and either tracrRNA or sgRNAs targeting Lzk. FIG. 41E shows survival of WT or SpCas9 knockin RGCs transfected with individual sgRNAs or pools of sgRNA targeting Dlk and/or Lzk. FIG. 41F shows normalized survival (SpCas9-WT) conferred by transfecting increasing amounts of sgRNA targeting each of the four transcription factors (Jun, Atf2, Sox11, Mef2a) alone or in combination and compared to transfection with negative control tracrRNA or positive control sgRNAs targeting Dlk/Lzk. FIG. 41H depicts survival (±SD) of SpCas9 RGCs, transfected with sgRNA, two days after adenoviral transduction to activate LZK signaling. *P<0.05 Mann-Whitney U test comparing Dlk/Lzk and transcription factor sgRNAs. FIG. 41G depicts a diagram showing the proposed pathway for RGC cell death following axon injury.

FIG. 42 (related to FIG. 61) contains 6 panels, A-F, showing that LZK is a mediator of cell death in primary RGCs.

FIG. 43 (related to FIG. 62) contains 5 panels, A-E, showing development of a flow cytometry-based assay to quantify RGC survival.

FIG. 44 (related to FIG. 63) contains 4 panels, A-D, showing whole-genome siRNA screen results.

FIG. 45 (related to FIGS. 64 and 65) contains 8 panels, A-H, showing knockdown of Mef2a, Puma and Atf2 and optic nerve injury leads to DLK-dependent MEF2A phosphorylation.

FIG. 46 (related to FIG. 66) contains 5 panels, A-E, showing whole-genome, sensitized siRNA screen results.

FIG. 47 (related to FIG. 68) contains 2 panels, A-B, showing CRISPR knockout of Dlk in primary RGCs.

FIG. 48 contains two panels, A-B, depicting survival graphs.

FIG. 49 depicts a table of tracer RNA, mm190, mm204, mm094, mm079, mm936, mm926, mm375, mm775, mm878, and mm942.

FIG. 50 shows the AAV2 construct size for targeting of CEP20. The construct size is 4,781 bp. The promoter is the H1 bidirectional (mouse). The Pol II terminator is SPA and the AAV serotype is AAV2.

FIG. 51 shows that LCA10 is caused by an intronic mutation in CEP290. The CEP290 gene is depicted. The IVS26 c.2991+1655 A>G mutation results in aberrant splicing and inclusion of an 128 bp cryptic Exon X (bottom).

FIG. 52 depicts the genomic organization of the H1RNA and PARP-2 locus. Shown above is a depiction of the PARP-2 gene (blue) transcribed toward the right and the H1RNA gene (orange) transcribed to the left drawn to scale. Below is an enlarged region of the promoter region for both genes.

FIG. 53 depicts the SpCas9 target site and the LCA10 mutation. The location of the A>G mutation is indicated in context with the 3′ end of Exon X (orange), and the SpCas9 target site (blue). The SpCas9 cutsite is depicted by the two arrows and critical nucleotides for splicing are boxed.

FIG. 54 depicts the High-fidelity/High-specificity SpCas9 Variants. The eSpCas9 point mutations are indicated in blue, and the spCas9-HF 1 point mutations are indicated in orange.

FIG. 55 depicts the Cas9 nickase approach. The nickase requires two closely opposed target sites (L and R; top) on opposite strands to generate a double-strand DNA break (below).

FIG. 56 comprises three panels, A-C, depicting the LCA10 CRISPR/AAV Therapeutics. FIG. 56A depicts a cartoon of the AAV virus and the packaging capacity.

FIG. 56B depicts a diagram for the SpCas9 targeting constructs, which include eSpCas9 and SpCas9-HF variants from the SA1. FIG. 56C depicts a diagram of the SaCas9 nickase with four gRNAs as described in SA2.

FIG. 57 contains eight panels, A-H. FIGS. 57A and 57B depict the genomic locus of CEP290 with the location of the deep intronic LCA10 mutation (A->G) indicated. This mutation causes the inclusion of a cryptic exon (Exon X) into the mRNA, resulting in a truncated protein. The A->G mutation can be targeted by a CRISPR-Cas9 site that falls over the mutation sequence. Targeting by this gRNA is expected to result in correct splicing, as indels formed in an around the splice junction can impair splicing of this pseudo exon. Bottom show the general strategy of targeting a dsDNA break to intronic region to restore normal CEP290 expression. FIG. 57C depicts the normal CEP290 gene (exons 25-28). FIG. 57D depicts the point mutation, which then results in the inclusion of the cryptic exon into CEP290. FIGS. 57E and 57F depict a reported strategy of trying to remove the point mutation sequence using the SaCas9 (because it is small enough to fit into AAV). This strategy seeks to remove a large section of the intron to remove the point mutation sequence. This is most likely because there are no SaCas9 sites near the point mutation. This strategy for using SaCas9 is likely to suffer two consequences: (1) lower efficiency, as two DNA cuts are required instead of one; (2) safety issues such as chromosomal translocations or rearrangements, and the potential for insertional mutagenesis to occur, and (3) the potential for more off targets. Conversely, using the bidirectional strategy, Cas9 (or Cas9 variants, or Cpf1, or Cpf1 variants) can be delivered by AAV, opening up many more targeting sites, and strategies to restore normal splicing for CEP290. FIGS. 57G and 57H depict cleavage of the CEP290 targeting site as measured by resistance to BmrI cleavage following transfection of a plasmid, or transduction by an AAV virus.

FIG. 58 depicts shows the AAV5 construct size for targeting of Rhodopsin in vivo. The construct size is 4,996 bp. The promoter is the H1 bidirectional (human). The Pol II terminator is SV40. The AAV serotype is AAV5.

FIG. 59 contains five panels, A-E, showing bidirectional expression in vivo following intravitreal injection. FIG. 59A and depict validation of the H1-bidirectional promoter construct in vivo. As shown on in FIG. 59A, a construct was generated using the H1 promoter was used to express an RNA (gRNA shown in black) and GFP simultaneously. GFP was used to provide a visual readout of pol II expression from the H1 promoter. The construct which was flanked by AAV ITR sequences, was then packaged into virus using the AAV2* (Y444F+Y500F+Y730F triple capsid mutation, indicated by the *), which has a preference for retinal ganglion cell transduction. The virus was delivered by intravitreal injection; a control injection using vehicle alone was delivered to control eye. 14 days post-injection, the mouse was sedated and visualized for GFP expression using a Micro III retinal imaging microscope. Diffuse GFP expression can be detected from the living mouse, as shown on the left panel. FIG. 59C shows no GPF expression. The mouse was euthanized and GFP expression was visualized from a retinal flat mount, indicating GFP expression in the Retinal ganglion cell layer, as would be expected using the AAV2* serotype. FIG. 59C shows the H1 bidirectional promoter was used to express GFP (and an empty gRNA), and packaged into either AAV2 or AAV5 virus. This experiment is to test H1 expression in different cell types and to validate cell-type tropism. FIG. 59D shows a cartoon illustration depicting the meaning of serotypes and tropism. Serotypes refer to the different “strains” of AAV, and tropism refers to the types of cells that a given strain can infect. This property provides an additional layer of safety as specific serotypes can be used to target the desired cell types. For example, in the retina, AAV serotypes are well-characterized and can be used to preferentially infect certain cells, even though they are surrounded by many different cell-types. In particular, AAV5 demonstrates photoreceptor specificity.

FIG. 60 contains two panels, A-B, showing retinal genome-editing by CRISPR in vivo and a strategy to target dominant alleles using SNPs. This example is for targeting the P23H mutation in rhodopsin. FIG. 60B shows CRISPR targeting of a dominant mutation in vivo (RHO P23H) depicting the use of SNPs for allelic specificity and inactivation in cis., and the use of engineered Cas9 variants to target P23H. On the left shows the breeding scheme using the P23H homozygous mouse on the C57BL strain, crossed with the wild-type inbred mouse strain CAST/EiJ. The CAST strain carries a non-synonymous SNP not carried in other strains, as shown by sequencing. This SNP does not change the WT Rhodopsin protein sequence, and can be used to discriminate between the WT rhodopsin sequence and the P23H sequence. The P23H point mutation (C->A) falls on the N of the NGG from the Cas9 PAM sequence, and thus the gRNA will target both the WT and P23H sequences equally. The CAST sequence carries an additional bas change that allows the gRNA to target the mutation and not the WT sequence.

FIG. 61 (related to FIG. 42) contains 11 panels, A-K, showing that LZK is a mediator of cell death in primary RGCs. FIG. 61A shows a comparison of CellTiter-Glo (“Survival (RLU)”) and Cellomics-based (“Viable RGCs”) quantification of RGCs at the time of plating. FIG. 61B-C shows capillary-based immunoassay (top) and quantification (bottom) of LZK in RGCs after transfection with siRNAs (B) or siPOOLs (C). FIG. 61D shows comparison of CellTiter-Glo (“Survival (RLU)”) and Cellomics-based (“Viable RGCs”) quantification of RGCs, transfected with Lzk siPOOL±Dlk siPOOL, 48 hours after colchicine. NS, non-significant by Mann-Whitney U test. FIG. 61E shows viable RGCs (calcein-AM-staining/ethidium homodimer-excluding; ±SD) quantified by automated fluorescent microscopy. FIG. 61F shows capillary-based immunoassay (top) and quantification (bottom) of LZK in RGCs one day after transfection with control or Lzk siPOOL and transduction with adenovirus expressing mouse siRNA resistant, human LZK cDNA or a GFP control. FIG. 61G shows survival (±SD) of WT RGCs transfected with Dlk/Lzk siPOOL, two days after reconstitution of LZK signaling with adenovirus expressing mouse siRNA-resistant, human LZK cDNA or a GFP control (“LZK reconstitution assay”). FIG. 61H-I shows representative photomicrographs (H) and quantification (I) of neurite length (average per neuron) in RGCs transfected with siPOOL, and stained with calcein-AM three days after immunopanning injury. FIG. 61J shows survival (±SD) of WT RGCs, transduced with adenovirus, two days after colchicine challenge. FIG. 61K shows co-immunoprecipitation assay from WT RGCs one day after immunopanning injury.

FIG. 62 (related to FIG. 43) contains 6 panels, A-F, showing development of a flow cytometry-based assay to quantify RGC survival. FIG. 62A depicts immunofluorescent staining of a representative retinal flatmount from an uninjured, wildtype C57BL/6 mouse. FIG. 62B depicts 2D plot of immunopanned, P0-3 mouse RGCs analyzed by FC immediately after immunopanning. FIG. 62C-D depicts representative 2D plots of uninjured retinas (C) or two weeks after ONC (D), analyzed by FC. Gates in (C) were used to show the percentage of TUBB3/SNCG double-positive cells that are doublepositive for Thy1.2/NeuN, or vice versa, in order to approximate the specificity and sensitivity, respectively, of the technique. The averages are shown below. FIG. 62E depicts FC-based quantification of surviving RGCs (±SD) at various timepoints after ONC or sham (n in parentheses). FIG. 62F depicts comparison of FC-based quantification of surviving RGCs (±SD) versus manual counting of immunostained flatmounts. In both cases, RGCs are identified by SNCG/TUBB3 double-staining. NS, non-significant Mann-Whitney U test.

FIG. 63 (related to FIG. 44) contains four panels, A-D. Whole-genome siRNA screen results. FIG. 63A-B show the rank order of the top 48 genes, arranged by Z-score, after correcting for the contribution by the seed sequence (A) or the top 14 gene as determined by Haystack analysis (B). Validated genes are shown in red. FIG. 63C-D depict the results of the secondary screening in which RGCs were transfected with four independent siRNAs targeting the top genes nominated by the seed-corrected (C) or Haystack (D) analyses. Dashed line shows the threshold of survival greater than 3SD from the negative control.

FIG. 64 (related to FIG. 45) contains five panels, A-E, showing knockdown of Mef2a, Puma and Atf2. FIG. 64A-B show capillary-based immunoassay with associated quantification (top, middle) or qPCR (bottom) performed on RGCs transfected with individual siRNAs (A) or siPOOLs (B). FIG. 64C shows capillary-based immunoassay of ATF2 from Atf2fl/fl RGCs transduced with adenovirus for three days. FIG. 64D shows survival (±SD) of RGCs, transduced with adenovirus, two days after a colchicine challenge. FIG. 64E shows a FC-based quantification of surviving RGCs, normalized to the uninjured control (±SD), two weeks after ONC or sham surgery. All eyes were injected with 109 vg AAV2-Cre two weeks prior to the surgery. *P<0.05, Mann-Whitney U test.

FIG. 65 (related to FIG. 45) contains two panels, A and B, showing optic nerve injury leads to DLK-dependent MEF2A phosphorylation. FIG. 65A shows merged immunofluorescent staining of representative retinal sections two days after ONC or sham surgery. FIG. 65B shows a 2D plot of representative WT or Dlkfl/fl retinas analyzed by FC two days after ONC or sham surgery.

FIG. 66 (related to FIG. 46), contains five panels, A-E, showing whole-genome, sensitized siRNA screen results. FIG. 66A shows a rank order of the top 48 candidate genes, arranged by Z-score, after correcting for the contribution by the seed sequence. Previously-validated genes are shown in red. FIG. 66B shows a scatter plot showing the seed-corrected activity for each minipool in the whole-genome library. Previously-validated genes are shown in red. FIG. 66C shows results of the secondary screening in which RGCs were transfected with four independent siRNAs targeting the top genes nominated by the seed-corrected analysis. Dashed line shows the threshold of survival greater than 3SD from the negative control. FIG. 66D shows a rank order of the top genes as determined by Haystack analysis. FIG. 66E shows results of secondary screening in which RGCs were transfected with four independent siRNAs targeting the top genes nominated by Haystack analysis. Dashed line shows the threshold of survival greater than 3SD from the negative control.

FIG. 67 shows the effect of transcription factors on neurite outgrowth. Quantification of neurite length (average per neuron; ±SD) in RGCs transfected with siPOOLs, and stained with calcein-AM three days after immunopanning injury. *P<0.05 Mann-Whitney U test.

FIG. 68 (related to FIG. 47) contains four panels, A-D, showing CRISPR knockout of Dlk in primary RGCs. FIG. 68A shows a BglII digest of PCR products, amplified from SpCas9 RGC genomic DNA, three days after transfection with tracrRNA or Dlk gRNA #4, and maintained in the presence of DLK/LZK inhibitor to avoid selection. The PCR regions included the D/k gRNA #4 target site or the top 14 off-targets predicted by http://crispr.mit.edu (all of which maintain the BglII site). FIG. 68B shows sequencing results of the Dlk #4 PCR product after subcloning. FIG. 68C shows survival of WT vs. SpCas9-expressing RGCs (±SD), transfected with tracrRNA or sgRNAs targeting Dlk and Lzk, in the absence or presence of neurotrophins (NTs, 50 ng/mL BDNF, 5 ng/mL GDNF, 5 ng/mL CNTF), two days after colchicine challenge. *P<0.05 Mann-Whitney U test. FIG. 68D shows difference in survival (SpCas9-WT; +SD) conferred by transfecting a second set of sgRNAs, targeting each of the four transcription factors, alone or in combination and compared to negative control tracrRNA.

FIG. 69 depicts pharmacologic inhibition of DLK and LZK, including by sunitinib, an FDA-approved small molecule inhibitor, promotes the survival of human ESC-derived RGCs. FIG. 69A depicts survival (±SD) of hESC-derived RGCs two days after a challenge with vehicle or colchicine (1 μM) in the presence of the DLK/LZK inhibitors tozasertib (1 μM), Genentech inhibitor 123 (0.1 μM) or a vehicle control. FIG. 69B depicts survival (±SD) of hESC-derived RGCs two days after a challenge with colchicine (1 μM) in the presence of increasing doses of sunitinib.

FIG. 70 depicts in vivo genome editing using one embodiment of a construct targeting rhodopsin.

FIG. 71 depicts in vivo genome editing using one embodiment of a construct targeting rhodospin. Cells from total retina were used to assay for cutting (not purified photoreceptors), and slight levels of cutting are detectable at 14 days, which increases at 28 days.

FIG. 72 depicts fusion constructs that employ the nuclease-dead version of Cas9 for the modulation of transcriptional regulation. These constructs can be delivered by AAV using the H1 bidirectional promoter.

FIG. 73 shows a schematic of the rhodopsin promoter and protein binding sites in the promoter region. By disrupting these interactions, one could repress transcription of that allele in cis, and by exploiting SNPs in the promoter region, one could delivery non-cutting versions of Cas9 to treat ADRP.

FIG. 74 depicts a schematic of the rhodopin promoter and RER region (top). Middle panel shows the PCR products from three different mouse cells/strains, which were Sanger sequenced. The identified SNPS are indicated but the lines on the bottom diagram. We have identified several regions that would allow us to exploit these sequence variations to modulate expression of rhodopsin in cis.

FIG. 75 depicts scan of a ˜2 kb region in the rhodopsin promoter region (from RER to the transcriptional start) for CRISPR targeting sites. We choose 6 sites near the RER and 6 sites near the proximal promoter region and assayed them for cutting (we used cutting efficiency as a proxy for identifying accessible regions allowing for high binding efficiency). We have identified several candidate sequences that could allow for modulation by dCas9 either through simple disruption of chromatin looping between the RER/proximal-promoter region, or through the use of dCas9 fusions (activator/repressor domains).

FIG. 76 depicts the Rho-GFP mouse to test cutting and non-cutting methods on partial human sequences. Below, an in vitro assay using purified photoreceptors from the Rho-GFP mouse and looking for the loss of GFP by CRISPR targeting.

FIG. 77 depicts reporter assay to test for nuclease-dead Cas9 constructs fused to either activator or repressor domains.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

Genome-editing technologies such as zinc fingers nucleases (ZFN) (Porteus, and Baltimore (2003) Science 300: 763; Miller et al. (2007) Nat. Biotechnol. 25:778-785; Sander et al. (2011) Nature Methods 8:67-69; Wood et al. (2011) Science 333:307) and transcription activator-like effectors nucleases (TALEN) (Wood et al. (2011) Science 333:307; Boch et al. (2009) Science 326:1509-1512; Moscou and Bogdanove (2009) Science 326:1501; Christian et al. (2010) Genetics 186:757-761; Miller et al. (2011) Nat. Biotechnol. 29:143-148; Zhang et al. (2011) Nat. Biotechnol. 29:149-153; Reyon et al. (2012) Nat. Biotechnol. 30:460-465) have empowered the ability to generate targeted genome modifications and offer the potential to correct disease mutations with precision. While effective, these technologies are encumbered by practical limitations as both ZFN and TALEN pairs require synthesizing large and unique recognition proteins for a given DNA target site. Several groups have recently reported high-efficiency genome editing through the use of an engineered type II CRISPR/Cas9 system that circumvents these key limitations (Cong et al. (2013) Science 339:819-823; Jinek et al. (2013) eLife 2:e00471; Mali et al. (2013) Science 339:823-826; Cho et al. (2013) Nat. Biotechnol. 31:230-232; Hwang et al. (2013) Nat. Biotechnol. 31:227-229). Unlike ZFNs and TALENs, which are relatively time consuming and arduous to make, the CRISPR constructs, which rely upon the nuclease activity of the Cas9 protein coupled with a synthetic guide RNA (gRNA), are simple and fast to synthesize and can be multiplexed. However, despite the relative ease of their synthesis, CRISPRs have technological restrictions related to their access to targetable genome space, which is a function of both the properties of Cas9 itself and the synthesis of its gRNA.

Cleavage by the CRISPR system requires complementary base pairing of the gRNA to a 20-nucleotide DNA sequence and the requisite protospacer-adjacent motif (PAM), a short nucleotide motif found 3 to the target site (Jinek et al. (2012) Science 337: 816-821). One can, theoretically, target any unique N₂₀-PAM sequence in the genome using CRISPR technology. The DNA binding specificity of the PAM sequence, which varies depending upon the species of origin of the specific Cas9 employed, provides one constraint. Currently, the least restrictive and most commonly used Cas9 protein is from S. pyogenes, which recognizes the sequence NGG, and thus, any unique 21-nucleotide sequence in the genome followed by two guanosine nucleotides (N₂₀NGG) can be targeted. Expansion of the available targeting space imposed by the protein component is limited to the discovery and use of novel Cas9 proteins with altered PAM requirements (Cong et al. (2013) Science 339: 819-823; Hou et al. (2013) Proc. Natl. Acad. Sci. U.S.A., 110(39):15644-9), or pending the generation of novel Cas9 variants via mutagenesis or directed evolution. The second technological constraint of the CRISPR system arises from gRNA expression initiating at a 5′ guanosine nucleotide. Use of the type III class of RNA polymerase III promoters has been particularly amenable for gRNA expression because these short non-coding transcripts have well-defined ends, and all the necessary elements for transcription, with the exclusion of the 1+nucleotide, are contained in the upstream promoter region. However, since the commonly used U6 promoter requires a guanosine nucleotide to initiate transcription, use of the U6 promoter has further constrained genomic targeting sites to GN₁₉NGG (Mali et al. (2013) Science 339:823-826; Ding et al. (2013) Cell Stem Cell 12:393-394). Alternative approaches, such as in vitro transcription by T7, T3, or SP6 promoters, would also require initiating guanosine nucleotide(s) (Adhya et al. (1981) Proc. Natl. Acad. Sci. U.S.A. 78:147-151; Melton et al. (1984) Nucleic Acids Res. 12:7035-7056; Pleiss et al. (1998) RNA 4:1313-1317).

The presently disclosed subject matter relates to the modification of a CRISPR/Cas9 system to target retinal degenerations, which uses the H1 promoter to express guide-RNAs (gRNA or sgRNA) (WO2015/19561, herein incorporated by reference in its entirety) that target retinal degeneration-related genes, such as, but not limited, to LCA10 CEP290 gene, rhodopsin, Dual Leucine Zipper Kinase (DLK), Leucine Zipper Kinase (LZK), JNK1-3, MKK4, MKK7, ATF2, JUN, MEF2A, SOX11, or PUMA. Such a modified CRISPR/Cas9 system can precisely target the pathogenic mutations in these retinal degenerations with greater efficacy, safety, and precision. Moreover, this modification comprising gRNAs retain the compact nature of the CRISPR/Cas9 H1 promoter system that allows for higher-resolution targeting of retinal degenerations over existing CRISPR, TALEN, or Zinc-finger technologies.

### I. Expression of Crispr Guide RNAs Targeting Retinal Degenerations Using the H1 Promoter

### A. Compositions

In some embodiments, the presently disclosed methods for treating retinal degenerations utilize a composition comprising a modification of a non-naturally occurring CRISPR system previously described in WO2015/195621 (herein incorporated by reference in its entirety). Such a modification uses certain gRNAs that target retinal degeneration-related genes, such as, but not limited, to LCA10 CEP290 gene, rhodopsin, Dual Leucine Zipper Kinase (DLK), Leucine Zipper Kinase (LZK), JNK1-3, MKK4, MKK7, ATF2, JUN, MEF2A, SOX11, or PUMA. In some embodiments, the composition comprises (a) a non-naturally occurring nuclease system (e.g., CRISPR) comprising one or more vectors comprising: i) a promoter (e.g., bidirectional H1 promoter) operably linked to at least one nucleotide sequence encoding a nuclease system guide RNA (gRNA), wherein the gRNA hybridizes with a target sequence of a DNA molecule in a cell of the subject, and wherein the DNA molecule encodes one or more gene products expressed in the cell; and ii) a regulatory element operable in a cell operably linked to a nucleotide sequence encoding a genome-targeted nuclease (e.g., Cas9 protein), wherein components (i) and (ii) are located on the same or different vectors of the system, wherein the gRNA targets and hybridizes with the target sequence and the nuclease cleaves the DNA molecule to alter expression of the one or more gene products. In some embodiments, the system is packaged into a single adeno-associated virus (AAV) particle. In some embodiments, the adeno-associated virus (AAV) may comprise any of the 51 human adenovirus serotypes (e.g., serotypes 2, 5, or 35). In some embodiments, the system inactivates one or more gene products. In some embodiments, the nuclease system excises at least one gene mutation. In some embodiments, the promoter comprises: a) control elements that provide for transcription in one direction of at least one nucleotide sequence encoding a gRNA; and b) control elements that provide for transcription in the opposite direction of a nucleotide sequence encoding a genome-targeted nuclease. In some embodiments, the Cas9 protein is codon optimized for expression in the cell. In some embodiments, the promoter is operably linked to at least one, two, three, four, five, six, seven, eight, nine, or ten gRNA. In some embodiments, the target sequence is a mutation in the CEP290 gene (e.g., LCA10 CEP290 gene). In some embodiments, the target sequence for CEP290 is selected from the group consisting of the nucleotide sequences set forth in SEQ ID NO: 1-109, 164-356, 735-738, or combinations thereof. In some embodiments, the target sequence comprises SEQ ID NOs: 1, 2, 3, and 4 operably linked. In some embodiments, the vector comprises the nucleotide sequence set forth in SEQ ID NO: 110. In some embodiments, the one or more gene products are rhodopsin. In some embodiments, the target sequence is a mutation in the rhodopsin gene. In some embodiments, the target sequence is a mutation at R135 of the rhodopsin gene (e.g., R135G, R135W, R135L). In some embodiments, the target sequence for rhodopsin R135 is selected from the group consisting of the nucleotide sequences set forth in SEQ ID NO: 111-126, or combinations thereof. In some embodiments, the gRNA sequence for rhodopsin R135 is selected from the group consisting of the nucleotide sequences set forth in SEQ ID NO: 127-142, or combinations thereof. In some embodiments, the one or more gene products are Dual Leucine Zipper Kinase (DLK), Leucine Zipper Kinase (LZK), JNK1-3, MKK4, MKK7, ATF2, JUN, MEF2A, SOX11, or PUMA or combinations thereof. In some embodiments, the mutation targets of glaucoma include, but not limited to, OPTN, TBK1, TMCO1, PMM2, GMDS, GAS7, FNDC3B, TXNRD2, ATXN2, CAV1/CAV2, p16INK4a, SIX6, ABCA1, AFAP1 and CDKN2B-AS. In some embodiments, the target sequence for glaucoma is selected from the group consisting of the nucleotide sequences set forth in SEQ ID NO: 143-163, or combinations thereof.

In some embodiments, the presently disclosed methods for treating retinal degenerations utilize a composition comprising a non-naturally occurring CRISPR system comprising one or more vectors comprising: a) an H1 promoter operably linked to at least one nucleotide sequence encoding a CRISPR system guide RNA (gRNA), wherein the gRNA hybridizes with a target sequence of a DNA molecule in a cell, and wherein the DNA molecule encodes one or more gene products expressed in the cell; and b) a regulatory element operable in a cell operably linked to a nucleotide sequence encoding a Cas9 protein, wherein components (a) and (b) are located on the same or different vectors of the system, wherein the gRNA targets and hybridizes with the target sequence and the Cas9 protein cleaves the DNA molecule to alter expression of the one or more gene products.

In some embodiments, the presently disclosed methods for treating retinal degenerations utilizes a composition comprising a non-naturally occurring CRISPR system comprising one or more vectors comprising: a) an H1 promoter operably linked to at least one nucleotide sequence encoding a CRISPR system guide RNA (gRNA), wherein the gRNA hybridizes with a target sequence of a DNA molecule in a eukaryotic cell, and wherein the DNA molecule encodes one or more gene products expressed in the eukaryotic cell; and b) a regulatory element operable in a eukaryotic cell operably linked to a nucleotide sequence encoding a Type-II Cas9 protein, wherein components (a) and (b) are located on the same or different vectors of the system, whereby the gRNA targets and hybridizes with the target sequence and the Cas9 protein cleaves the DNA molecule, and whereby expression of the one or more gene products is altered. In one aspect, the target sequence can be a target sequence that starts with any nucleotide, for example, N₂₀NGG. In some embodiments, the target sequence comprises the nucleotide sequence AN₁₉NGG. In some embodiments, the target sequence comprises the nucleotide sequence GN₁₉NGG. In some embodiments, the target sequence comprises the nucleotide sequence CN₁₉NGG. In some embodiments, the target sequence comprises the nucleotide sequence TN₁₉NGG. In some embodiments, the target sequence comprises the nucleotide sequence AN₁₉NGG or GN₁₉NGG. In another aspect, the Cas9 protein is codon optimized for expression in the cell. In another aspect, the Cas9 protein is codon optimized for expression in the eukaryotic cell. In a further aspect, the eukaryotic cell is a mammalian or human cell. In yet another aspect, the expression of the one or more gene products is decreased.

In some embodiments, the presently disclosed methods for treating retinal degenerations utilizes a composition comprising a non-naturally occurring CRISPR system comprising a vector comprising a bidirectional H1 promoter, wherein the bidirectional H1 promoter comprises: a) control elements that provide for transcription in one direction of at least one nucleotide sequence encoding a CRISPR system guide RNA (gRNA), wherein the gRNA hybridizes with a target sequence of a DNA molecule in a eukaryotic cell, and wherein the DNA molecule encodes one or more gene products expressed in the eukaryotic cell; and b) control elements that provide for transcription in the opposite direction of a nucleotide sequence encoding a Type-II Cas9 protein, whereby the gRNA targets and hybridizes with the target sequence and the Cas9 protein cleaves the DNA molecule, and whereby expression of the one or more gene products is altered. In one aspect, the target sequence can be a target sequence that starts with any nucleotide, for example, N₂₀NGG. In some embodiments, the target sequence comprises the nucleotide sequence AN₁₉NGG. In some embodiments, the target sequence comprises the nucleotide sequence GN₁₉NGG. In some embodiments, the target sequence comprises the nucleotide sequence CN₁₉NGG. In some embodiments, the target sequence comprises the nucleotide sequence TN₁₉NGG. In some embodiments, the target sequence comprises the nucleotide sequence AN₁₉NGG or GN₁₉NGG. In another aspect, the Cas9 protein is codon optimized for expression in the cell. In another aspect, the Cas9 protein is codon optimized for expression in the eukaryotic cell. In a further aspect, the eukaryotic cell is a mammalian or human cell. In yet another aspect, the expression of the one or more gene products is decreased.

In some embodiments, the CRISPR complex comprises one or more nuclear localization sequences of sufficient strength to drive accumulation of the CRISPR complex in a detectable amount in the nucleus of a cell (e.g., eukaryotic cell). Without wishing to be bound by theory, it is believed that a nuclear localization sequence is not necessary for CRISPR complex activity in eukaryotes, but that including such sequences enhances activity of the system, especially as to targeting nucleic acid molecules in the nucleus. In some embodiments, the CRISPR enzyme is a type II CRISPR system enzyme. In some embodiments, the CRISPR enzyme is a Cas9 enzyme. In some embodiments, the Cas9 enzyme is S. pneumoniae, S. pyogenes, or S. thermophilus Cas9, and may include mutated Cas9 derived from these organisms. The enzyme may be a Cas9 homolog or ortholog.

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses). Viral vectors also include polynucleotides carried by a virus for transfection into a host cell.

Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the presently disclosed subject matter in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed.

Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel (1990) Gene Expression Technology: Methods in Enzymology 185, Academic Press, San Diego, Calif. Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific.

In some embodiments, a vector comprises one or more pol III promoters, one or more pol 11 promoters, one or more pol I promoters, or combinations thereof. Examples of pol ITT promoters include, but are not limited to, U6 and H1 promoters. Examples of pol IT promoters include, but are not limited to, the retroviral Rous sarcoma virus (RSV) LTR promoter (optionally with the RSV enhancer), the cytomegalovirus (CMV) promoter (optionally with the CMV enhancer) (e.g., Boshart et al. (1985) Cell 41:521-530), the SV40 promoter, the dihydrofolate reductase promoter, the β-actin promoter, the phosphoglycerol kinase (PGK) promoter, and the EF1α promoter.

Also encompassed by the term “regulatory element” are enhancer elements, such as WPRE; CMV enhancers; the R-U5′ segment in LTR of HTLV-I (Takebe et al. (1988) Mol. Cell. Biol. 8:466-472); SV40 enhancer; and the intron sequence between exons 2 and 3 of rabbit β-globin (O'Hare et al. (1981) Proc. Natl. Acad. Sci. USA. 78(3):1527-31). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.). Advantageous vectors include lentiviruses and adeno-associated viruses, and types of such vectors can also be selected for targeting particular types of cells.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

In aspects of the presently disclosed subject matter the terms “chimeric RNA”, “chimeric guide RNA”, “guide RNA”, “single guide RNA” and “synthetic guide RNA” are used interchangeably and refer to the polynucleotide sequence comprising the guide sequence. The term “guide sequence” refers to the about 20 bp sequence within the guide RNA that specifies the target site and may be used interchangeably with the terms “guide” or “spacer”.

As used herein the term “wild type” is a term of the art understood by skilled persons and means the typical form of an organism, strain, gene or characteristic as it occurs in nature as distinguished from mutant or variant forms.

As used herein the term “variant” should be taken to mean the exhibition of qualities that have a pattern that deviates from what occurs in nature.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 700%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part 1, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non amino acids. The terms also encompass an amino acid polymer that has been modified; for example, disulfide bond formation, glycosylation, lipidation, acetylation, phosphorylation, or any other manipulation, such as conjugation with a labeling component.

As used herein the term “amino acid” includes natural and/or unnatural or synthetic amino acids, including glycine and both the D or L optical isomers, and amino acid analogs and peptidomimetics.

The practice of the present presently disclosed subject matter employs, unless otherwise indicated, conventional techniques of immunology, biochemistry, chemistry, molecular biology, microbiology, cell biology, genomics and recombinant DNA, which are within the skill of the art (Sambrook, Fritsch and Maniatis (1989) Molecular Cloning: A Laboratory Manual, 2nd edition; Ausubel et al., eds. (1987) Current Protocols in Molecular Biology); MacPherson et al., eds. (1995) Methods in Enzymology (Academic Press, Inc.): PCR 2: A Practical Approach); Harlow and Lane, eds. (1988) Antibodies, A Laboratory Manual; Freshney, ed. (1987) Animal Cell Culture).

Several aspects of the presently disclosed subject matter relate to vector systems comprising one or more vectors, or vectors as such. Vectors can be designed for expression of CRISPR transcripts (e.g. nucleic acid transcripts, proteins, or enzymes) in prokaryotic or eukaryotic cells. For example, CRISPR transcripts can be expressed in bacterial cells such as Escherichia coli, insect cells (using baculovirus expression vectors), yeast cells, or mammalian cells. Suitable host cells are discussed further in Goeddel (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Vectors may be introduced and propagated in a prokaryote. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins.

Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson (1988) Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A. respectively, to the target recombinant protein.

Examples of suitable inducible non-fusion E. coli expression vectors include pTrc (Amrann et al. (1988) Gene 69:301-315) and pET 11d (Studier et al. (1990) Gene Expression Technology: Methods in Enzymology 185, Academic Press, San Diego, Calif.).

In some embodiments, a vector is a yeast expression vector. Examples of vectors for expression in yeast Saccharomyces cerevisae include pYepSec1 (Baldari, et al. (1987) EMBO J. 6: 229-234), pMFa (Kuijan and Herskowitz (1982) Cell 30: 933-943), pJRY88 (Schultz et al. (1987) Gene 54: 113-123), pYES2 (Invitrogen Corporation, San Diego, Calif.), and picZ (InVitrogen Corp, San Diego, Calif.).

In some embodiments, a vector is capable of driving expression of one or more sequences in mammalian cells using a mammalian expression vector. Examples of mammalian expression vectors include pCDM8 (Seed (1987) Nature 329: 840) and pMT2PC (Kaufman et al. (1987) EMBO J. 6; 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook et al. (1989) Molecular Cloning: A Laboratory Manual 2nd ed., Cold Spring Harbor Laboratory, Cold Spring Harbor Laboratory Press, Cold Spring Harbor, N.Y..

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1: 268-277), lymphoid-specific promoters (Calame and Eaton (1988) Adv. Immunol. 43: 235-275), in particular promoters of T cell receptors (Winoto and Baltimore (1989) EMBO J. 8: 729-733) and immunoglobulins (Baneiji et al. (1983) Cell 33: 729-740: Queen and Baltimore (1983) Cell 33: 741-748), neuron-specific promoters (e.g., the neurofilament promoter; Byrne and Ruddle (1989) Proc. Natl. Acad. Sci. USA 86: 5473-5477), pancreas-specific promoters (Edlund et al. (1985) Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine box promoters (Kessel and Gruss (1990) Science 249: 374-379) and the α-fetoprotein promoter (Campes and Tilghman (1989) Genes Dev. 3: 537-546).

In some embodiments, a regulatory element is operably linked to one or more elements of a CRISPR system so as to drive expression of the one or more elements of the CRISPR system. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al. (1987) J. Bacteriol., 169:5429-5433; and Nakata et al. (1989) J. Bacteriol., 171:3553-3556), and associated genes. Similar interspersed SSRs have been identified in Haloferax mediterranei, Streptococcus pyogenes, Anabaena, and Mycobacterium tuberculosis (Groenen et al. (1993) Mol. Microbiol., 10:1057-1065; Hoe et al. (1999) Emerg. Infect. Dis., 5:254-263; Masepohl et al. (1996) Biochim. Biophys. Acta 1307:26-30; and Mojica et al. (1995) Mol. Microbiol., 17:85-93). The CRISPR loci typically differ from other SSRs by the structure of the repeats, which have been termed short regularly spaced repeats (SRSRs) (Janssen et al. (2002) OMICS. J. Integ. Biol., 6:23-33; and Mojica et al. (2000) Mol. Microbiol., 36:244-246) In general, the repeats are short elements that occur in clusters that are regularly spaced by unique intervening sequences with a substantially constant length (Mojica et al. (2000) Mol. Microbiol., 36:244-246). Although the repeat sequences are highly conserved between strains, the number of interspersed repeats and the sequences of the spacer regions typically differ from strain to strain (van Embden et al. (2000) J. Bacteriol., 182.2393-2401). CRISPR loci have been identified in more than 40 prokaryotes (e.g., Jansen et al. (2002) Mol. Microbiol., 43:1565-1575, and Mojica et al. (2005) J. Mol. Evol. 60:174-82) including, but not limited to Aeropyrum, Pyrobaculum, Sulfolobus, Archaeoglobus, Halocarcula, Methanobacterium, Methanococcus, Methanosarcina, Methanopyrus, Pyrococcus, Picrophilus, Thermoplasma, Corynebacterium, Mycobacterium, Streptomyces, Aquifex, Porphyromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Ascherichia, Legionella, Alethylococcus, Pasteurella, Photobacterium, Salmonella, Xanthomonas, Yersinia, Treponema, and Thermotoga.

In general, “CRISPR system” refers collectively to transcripts and other elements involved in the expression of or directing the activity of CRISPR-associated (“Cas”) genes, including sequences encoding a Cas gene, a guide sequence (also referred to as a “spacer” in the context of an endogenous CRISPR system), or other sequences and transcripts from a CRISPR locus. In some embodiments, one or more elements of a CRISPR system is derived from a type 1, type II, or type Ill CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system).

In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the presently disclosed subject matter, an exogenous template polynucleotide may be referred to as an editing template. In an aspect of the presently disclosed subject matter the recombination is homologous recombination.

In some embodiments, a vector comprises one or more insertion sites, such as a restriction endonuclease recognition sequence (also referred to as a “cloning site”). In some embodiments, one or more insertion sites (e.g. about or more than about 1, 2, 3, 4, 5, 6, 7, 8, 9, 10, or more insertion sites) are located upstream and/or downstream of one or more sequence elements of one or more vectors. When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to a cell.

In some embodiments, a vector comprises a regulatory element operably linked to an enzyme-coding sequence encoding a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, Cas13a, C2c2, C2c3, homologs thereof, or modified versions thereof. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes or S. pneumoniae.

In some embodiments, the CRISPR enzyme directs cleavage of one or both strands at the location of a target sequence, such as within the target sequence and/or within the complement of the target sequence In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence.

In some embodiments, an enzyme coding sequence encoding a CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura et al. (2000) Nucl. Acids Res. 28:292. Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length.

The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. For example, in some embodiments, the target sequence of LCA is selected from the group consisting of SEQ ID NO: 1, 2, 3, 4, 5, and 6; or combinations thereof. SEQ ID NO: 1, 2, 3, 4, 5, and 6; or combinations thereof can result in ˜1 kb deletion removing the cryptic Exon D from CEP290. Combining SEQ ID NO: 1, 2, 3, 4, 5, and 6 with a modified form of Cas9, such as D10A nickase, may provide a safe and effective therapeutic approach. An exemplary saCas9 construct with four gRNAs is set forth in SEQ ID NO: 110. Additional target sequences of LCA may be selected from the nucleotide sequences set forth in SEQ ID NOs: 7-109. In some embodiments, the target sequences of ADRP is selected from the group consisting of SEQ ID NO: 111-126, or combinations thereof. In some embodiments, the gRNA sequences of ADRP is selected from the group consisting of SEQ ID NO: 127-142, or combinations thereof. In some embodiments, the mutation targets of glaucoma include, but not limited to, OPTN, TBK1, TMCO1, PMM2, GMDS, GAS7, FNDC3B, TXNRD2, ATXN2, CAV1/CAV2, p16INK4a, SIX6, ABCA1, AFAP1 and CDKN2B-AS. In some embodiments, the target sequences of glaucoma is selected from the group consisting of SEQ ID NO: 143-163, or combinations thereof. In some embodiments for the methods to treat glaucoma, the non-naturally occurring CRISPR system comprises H1 promoter to express an mCherry-histone 2b fusion in the Pol II direction in combination with at least one gRNA, e.g., directed to the Dlk, Lzk, or other upstream or downstream components of RGC survival pathway identified using the screening methods provided here.

In some embodiments, the target sequence may be 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% homologous to the nucleotide sequences set forth in SEQ ID NO: 1-738, or 788-1397.

The term “homologous” refers to the “% homology” and is used interchangeably herein with the term “% identity” herein, and relates to the level of nucleic acid sequence identity when aligned using a sequence alignment program.

For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to about, 80%, 81%, 82%, 83%, 84%, 85%, 86%, 87%, 88%, 89%, 90%, 91%, 92%, 93%, 94%, 95%, 96%, 97%, 98%, 99.1%, 99.2%, 99.3%, 99.4%, 99.5%, 99.6%, 99.7%, 99.8% or more sequence identity to the nucleotide sequences set forth in SEQ ID NO: 1-1400.

In some embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, VS tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US20110059502, incorporated herein by reference. In some embodiments, a tagged CRISPR enzyme is used to identify the location of a target sequence.

In an aspect of the presently disclosed subject matter, a reporter gene which includes but is not limited to glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into a cell to encode a gene product which serves as a marker by which to measure the alteration or modification of expression of the gene product. In a further embodiment of the presently disclosed subject matter, the DNA molecule encoding the gene product may be introduced into the cell via a vector. In a preferred embodiment of the presently disclosed subject matter the gene product is luciferase. In a further embodiment of the presently disclosed subject matter the expression of the gene product is decreased.

Generally, promoter embodiments of the present presently disclosed subject matter comprise: 1) a complete Pol III promoter, which includes a TATA box, a Proximal Sequence Element (PSE), and a Distal Sequence Element (DSE); and 2) a second basic Pol III promoter that includes a PSE and TATA box fused to the 5′ terminus of the DSE in reverse orientation. The TATA box, which is named for its nucleotide sequence, is a major determinant of Pol III specificity. It is usually located at a position between nt. −23 and −30 relative to the transcribed sequence, and is a primary determinant of the beginning of the transcribed sequence. The PSE is usually located between nt. −45 and −66. The DSE enhances the activity of the basic Pol III promoter. In the H1 promoter, there is no gap between the PSE and the DSE.

Bidirectional promoters consists of: 1) a complete, conventional, unidirectional Pol III promoter that contains 3 external control elements: a DSE, a PSE, and a TATA box; and 2) a second basic Pol III promoter that includes a PSE and a TATA box fused to the 5′ terminus of the DSE in reverse orientation. The TATA box, which is recognized by the TATA binding protein, is essential for recruiting Pol III to the promoter region. Binding of the TATA binding protein to the TATA box is stabilized by the interaction of SNAPc with the PSE. Together, these elements position Pol III correctly so that it can transcribe the expressed sequence. The DSE is also essential for full activity of the Pol Ill promoter (Murphy et al. (1992) Mol. Cell Biol. 12:3247-3261; Mittal et al. (1996) Mol. Cell Biol. 16:1955-1965; Ford and Hernandez (1997) J. Biol. Chem., 272:16048-16055; Ford et al. (1998) Genes, Dev., 12:3528-3540; Hovde et al. (2002) Genes Dev. 16:2772-2777). Transcription is enhanced up to 100-fold by interaction of the transcription factors Oct-1 and/or SBF/Staf with their motifs within the DSE (Kunkel and Hixon (1998) Nucl. Acid Res., 26:1536-1543). Since the forward and reverse oriented basic promoters direct transcription of sequences on opposing strands of the double-stranded DNA templates, the positive strand of the reverse oriented basic promoter is appended to the 5′ end of the negative strand of the DSE. Transcripts expressed under the control of the H1 promoter are terminated by an unbroken sequence of 4 or 5 T's.

In the H1 promoter, the DSE is adjacent to the PSE and the TATA box (Myslinski et al. (2001) Nucl. Acid Res. 29:2502-2509). To minimize sequence repetition, this promoter was rendered bidirectional by creating a hybrid promoter, in which transcription in the reverse direction is controlled by appending a PSE and TATA box derived from the U6 promoter. To facilitate construction of the bidirectional H1 promoter, a small spacer sequence may also inserted between the reverse oriented basic promoter and the DSE.

In some embodiments, bidirectional promoters include, but not limited to, H1, RPPH1-PARP2 (Human), SRP-RPS29, 7sk1-GSTA4, SNAR-G-1-CGB1, SNAR-CGB2, RMRP-CCDC 107, tRNA(Lys)-ALOXE3, RNU6-9-MED 16: tRNA (Gly)-DPP9, RNU6-2-THEM259, or SNORD13-C8orf41.

In some embodiments, the H1 promoter comprise the nucleotide sequence set forth in SEQ ID NO: 787.

H1_wt:
(SEQ ID NO: 787)
GGAATTCGAACGCTGACGTCATCAACCCGCTCCAAGGAATCGCGGGCCCA
GTGTCACTAGGCGGGAACACCCAGCGCGCGTGCGCCCTGGCAGGAAGATG
GCTGTGAGGGACAGGGGAGTGGCGCCCTGCAATATTTGCATGTCGCTATG
TGTTCTGGGAAATCACCATAAACGTGAAATGTCTTTGGATTTGGGAATCT
TATAAGTTCTGTATGAGACCACTTTTTCCC

In some embodiments, orthologous bidirectional promoters include, but not limited to, RPPH1-PARP2 (Mouse) or RPPH1-PARP2 (Rat), or those derived from Ailuropoda melanoleuca, Bos taurus, Callithrix jacchus, Canis familiaris, Cavia porcellus, Chlorocebus sabaeus, Choloepus hoffmanni, Dasypus novemcinctus, Dipodomys ordii, Equus caballus, Erinaceus europaeus, Felis catus, Gorilla gorilla, Homo sapiens, Ictidomys tridecemlineatus, Loxodonta africana, Macaca mulatta, Mus musculus, Mustela putorius furo, Myotis lucifugus, Nomascus leucogenys, Ochotona princeps, Oryctolagus cuniculus, Otolemur garnettii, Ovis aries, Pan troglodytes, Papio anubis, Pongo abelii, Procavia capensis, Pteropus vampyrus, Rattus norvegicus, Sus scrofa, Tarsius syrichta, Tupaia belangeri, Tursiops truncatus, Vicugna pacos.

TABLE 1
Examples of compact bidirectional promoters

| ncRNA gene | Protein gene | Distance |
|---|---|---|
| RPPH1 | PARP2 | 230 bp |
| SRP | RPS29 | 233 bp |
| 7sk1 | GSTA4 | 239 bp |
| SNAR-G1 | CGB1 | 308 bp |
| SNAR | CGB2 | 308 bp |
| RMRP | CCDC107 | 361 bp |
| tRNA(Lys) | ALOXE3 | 376 bp |
| RNU6-9 | MED16 | 412 bp |
| tRNA (Gly) | DPP9 | 484 bp |
| SNORD13 | C8orf41 | 847 bp |
| RNU6-2 | THEM259 | 525 bp |

RPPH1-PARP2 (Human):
(SEQ ID NO: 739)
GGAATTCGAACGCTGACGTCATCAACCCGCTCCAAGGAATCGCGGGCCCA
GTGTCACTAGGCGGGAACACCCAGCGCGCGTGCGCCCTGGCAGGAAGATG
GCTGTGAGGGACAGGGGAGTGGCGCCCTGCAATATTTGCATGTCGCTATG
TGTTCTGGGAAATCACCATAAACGTGAAATGTCTTTGGATTTGGGAATCT
TATAAGTTCTGTATGAGACCACTTTTTCCC
SRP-RPS29:
(SEQ ID NO: 740)
CTTGCTCTCAGCAGTGCAACGAGGTAAAAGGAAGAAGCTGGCCCACGCAT
GCGCTCTTCAAATTTTTGAGACAGTTTACCCAGAATGCAGTGCTCAAAGG
AAACGCGTGCGCAGTGTGGTCAGGTTGTTTCGCTGGGTGAGTAAAATGAA
ATCTTAGAGGCGTTGTGGGCTGGCCCAGTTGATGACGTCACCATACCACA
GCTTCTAGTGCTATTCTGCGCCGGTATCCGACC
7sk1-GSTA4:
(SEQ ID NO: 741)
AGTATTTAGCATGCCCCACCCATCTGCAAGGCATTCTGGATAGTGTCAAA
ACAGCCGGAAATCAAGTCCGTTTATCTCAAACTTTAGCATTTTGGGAATA
AATGATATTTGCTATGCTGGTTAAATTAGATTTTAGTTAAATTTCCTGCT
GAAGCTCTAGTACGATAAGCAACTTGACCTAAGTGTAAAGTTGAGACTTC
CTTCAGGTTTATATAGCTTGTGCGCCGCTTGGGTACCTC
SNAR-G-1-CGB1:
(SEQ ID NO: 742)
GTCTCTCTCTTAGCGGGATATCTTCCGCAAGCACTGGGAATGTGGACATG
GAAAGTAAATTGAGTCTCCGTGGGGGAGTGAGACAGGGAGTGAGGGGTGT
TGGACGCGGCACGGGAACCTGGCCAGAGTCAGCGGACCCAATTGGCTGCT
CTCTCTCAGATGCAGTTCCCCTTCCTCCCTCCAGGGGGCGCCACGGAACG
CAGGGCCCTCACTGGCCCTGGGGACTGGGTGACGTCAGGGATGAGCCTCT
TGTGATTGGCTCCATCACCCTGCGTAAGATCAAAGGGAAGAAAGGATGGG
CCCGACAA
SNAR-CGB2:
(SEQ ID NO: 743)
GTCTCTCTCTTAGCGGGATATCTTCCGCAAGCACTGGGGATGTGGACATG
GAAAGTAAATTGAGTCTCCGTGGGGGAGTGAGACAGGGAGTGAGGGGTGT
TGGACGCGGCACGGGAACCCGGCCGGAGTCAGCGGACCCAATTGGCTGCT
CTCTCTCAGATACAGTTCCCCTTCCTCCCTCCAGGGGGCGCCACGGAACG
CAGGGCCCTCACTGGCCCTGGGGACTGGGTGACGTCAGGGGTGAGCCTCT
CCTGATTGGCTCCATCACCCTGCGTAAGGTCAAAAGGAAGAAAGGAGATC
CCCGACAC
RMRP-CCDC107:
(SEQ ID NO: 744)
TGCCGGCCCACGGGTGGAGGGATCGGGCGGGCGGTGCCGAAGCGGTCCGG
CATTGGCCGGCCGCCCCAACGCGCACGCGCACGCGAGCAGGCCGGCCGGC
TCCGGGGAGGCCACGCCCACTCCCCGTAGGGCGGGGCCAGACCATATTTG
CATAAGATAGTGTCATTCTAGCTTTCCTGTATTTGTTCATTTCGTGTCTA
TTAGCTATTCTGCTAGCCACAATGCCTCTGAAAGCCTATAGTCTTAGAAA
GTTATGCCCGAAAACGGTTTTTTTAATCTCACGCCACCAACTTTCTCACC
CTAATCATAAAACACAATTTCTTTAGGGCTATAAAATACTACTCTGTGAA
GCTGAGGACGT
tRNA(Lys)-ALOXE3:
(SEQ ID NO: 745)
TCTTTCCGCTCCAGGACCGCCCTGGGCCTGCAGGATCCTGGGCGGGAGCC
CAGGTGTCCGGGATCTGGGCCACTAGGGACTGGGGAGGAACCTCTCAGAG
AAGCCCATAGCCCGCAGCGGCCCCGCGCGGCCGGTTCCGGCGCCGCACTG
TTCCAGCCTCTACTATGGTACAGTCCCTGCGTCGCAGCCTCGGCGGGGGC
TCTAAGAACGGGAGGCAGAAAAAGCTCAATCAGCAGCAGGCGAGCTTCAC
CCGCTGCTTCCAAATCTGTGCCAAAATATTCTATGCTGCACAGATAAAAT
CCTCTGTCGGTTCTACAAGCCTGGCTTTTCCTATAGAGAACCCTCTTATA
AGCAAAAAGTAAAGCTCTCGTGAAGA
RNU6-9-MED16:
(SEQ ID NO: 746)
GAGGGCAGTCACCAGCTCCTGGCCCGTGCGCCAAGCTCAGCGGGCGTCCG
CGGTGCGATCTTCCCTAGCGCCTCGGGTCTGGCGCCGCCATCTTCCTCGG
TAACAACCAGTCGCCTGAGGCGTGGGGCCGCCTCCCAAAGACTTCTGGGA
GGGCGGTGCGGCTCAGGCTCTGCCCCGCCTCCGGGGCTATTTGCATACGA
CCATTTCCAGTAATTCCCAGCAGCCACCGTAGCTATATTTGGTAGAACAA
CGAGCACTTTCTCAACTCCAGTCAATAACTACGTTAGTTGCATTACACAT
TGGGCTAATATAAATAGAGGTTAAATCTCTAGGTCATTTAAGAGAAGTCG
GCCTATGTGTACAGACATTTGTTCCAGGGGCTTTAAATAGCTGGTGGTGG
AACTCAATATTC
tRNA (Gly)-DPP9:
(SEQ ID NO: 747)
TAACCGCTCAGCTGACCTCAGGAGGGCAGGGGTGCCTTCTAAAGGGTCCA
GAGAGCCTCCATTCCAGCTGCAGGCGTGGGACACAGACCGGGACGTGGGG
CGGCGGCCGGACTGGGCAGGTCGTCCCGGGTCCAGCGGCGCCTCACGGTC
GCGGCTCCATGCCCGGGACTGCGACCCCGGAAGTGGCGGGAGCGGGGGAC
GACAGCCGCGGCGGACACAGGGGACCCGCCGGCTCAGGCACCTTTGACCC
GGAAGTTGAGCGACCCAGGCGGCGGCCTGGGATTGGACACCACCAGGCAC
GTACCAAGGCGTCCGCGGCGCTTGGGGGGGAGCCCGCGGCGCGGCGGCCT
AAGGTGCGTAACGCCCCATGAACGACATCTTCCGGTGGGTTAGGGAGAGA
CACCCCCCTGTGACTTGGTATCACTCAGTCAAACCCATGATCCCCCACTA
TTAAGGATATCCGGAGAGGATGCTACCTATCAGG
SNORD13-C8orf41:
(SEQ ID NO: 748)
TCCTGACTGCAGCACCAGAAGGCTGGTCTCTCCCACAGAACGAGGATGGA
GGCGGGGAGGGATCCGTTGAAGAGGGAAGGAGCGATCACCCAAAGAGAAC
TAAAATCAAATAAAATAAAACAGAGAGATGTCTTGGAGGAGGGGGCGAGT
CTGACCGGGATAAGAATAAAGAGAAAGGGTGAACCCGGGAGGCGGAGTTT
GCAGTGAGCCGAGATCGCGCCACTGCACTCCAGCCTGGGCGACAGAGTGA
GACTCCGTCTCAGTAAAAAAAAAAAAAAAAAAAAGAATAAAGAGGAAAGG
ACGCAAGAAAGGGAAAGGGGACTCTCAGGGAGTAAAAGAGTCTTACACTT
TTAACAGTGACGTTAAAAGACTACTGTTGCCTTTCTGAAGACTAAAAAGA
AAAAAAACTTAAAAATTTAAAGAAATAAACTTCTGAGCCATGTCACCAAC
TTAACCACCCCCAGGTACCTGCAACGGCTCGCGCCCGCCGGTGTCTAACA
GGATCCGGACCTAGCTCATATTGCTGCCGCAAAACGCAAGGCTAGCTTCC
GCCAGTACTGCCGCAACACCTTCTTATTTCACGACGTATGGTCGTAAAGC
AATAAAGATCCAGGCTCGGGAAAATGACGGAGAGGTGGAACTATAGAGAA
TAAATTTGCATATATAATAATCCGCTCGCTAATTGTGTTTCTGTTTTCCT
TTGCTAAGGTAGAAACAAAAGAATAATCACAGAATCTCAGTGGGACTTTG
AAAATATCCAGGATTTTATACGTGAAGAATGGATGTATCGCATTACGGTA
GTCACCCTATGTGTAAATTAGTGGCACATACTTGGCACTCCTTAATGTCA
ACTATAAGATG
RNU6-2-THEM259;
(SEQ ID NO: 749)
GCCTCCCAGCGTCGCGCCCTAACGACCCGCAAGTGTCCGAGGGCGCCTCC
CGGCCGCCATCGGCCGCCCTCGCAGCCGCCGCTCTCCTCACGGCCTCCCG
GCCGCCGCCGCCATCTTCCGCTTTCTCGTCCGGCTGCGGCGCTGCTGACG
CTAGCGAGTCGCCACGCCGGGCAAGAGCGGCCCCCCTGCGCCCGCAGAGA
ACGCTGGGATGCCAGCGGCGCCCGCGGAGGCCTCACCCCCTACCTCGGCC
GCTCCAGGGGGCGGGCCTGCATCTGGGCCACCTCTTTTGCATATTGGCAC
CCACAATCCACCGCGGCTATGAGGCCAGTATAAGGCGGTAAAATTACGAT
AAGATATGGGATTTTACGTGATCGAAGACATCAAAGTAAGCGTAAGCACG
AAAGTTGTTCTGCAACATACCACTGTAGGAAATTATGCTAAATATGAAAC
CGACCATAAGTTATCCTAACCAAAAGATGATTTGATTGAAGGGCTTAAAA
TAGGTGTGACAGTAACCCTTGAGTC

TABLE 6
Examples of orthologous bidirectional promoters

| ncRNA gene | Protein gene | Distance | Organism |
|---|---|---|---|
| RPPH1 | PARP2 | 230 bp | Human |
| RPPH1 | PARP2 | 172 bp | Mouse |
| RPPH1 | PARP2 | 201 bp | Rat |

RPPH1-PARP2 (Mouse):
(SEQ ID NO: 750)
CGCTCTTGAAGGACGACGTCATCATCCCTTGCCCGGATGCGCGGGCTTCT
TGTCTAGCACAGGAGCCTGGGGTAGAGCGCATGCAAATTACGCGCTGTGC
TTTGTGGGAAATCACCCTAAACGAAAAATTTATTCCTCTTTCGAGCCTTA
TAGTGGCGGCCGGTCTACATCC
RPPH1-PARP2 (Rat):
(SEQ ID NO: 751)
GGCTGATGAGCTTCCCCCGCCCACTAGGAGTGTGAAGACCTGCCGCCATA
ATAAGACTCCAAAAGACAGTGAATTTAACACTTACGGTGACTTCCCACAA
AGCACAGCGTGTAATTTGCATGCGCTCTAGCCCAGGCTCCAGCTCCGGAC
CAGAAGCCCGCGCATCCCGGCAAAGGGTGATGACGTCGTCCTTCAAGCGC
T

TABLE 7
Examples of Orthologous H1 sequences

mus_musculus
TTCAGGATGTAGACCGGCCGCCACTATAAGGCTCGAAAGAGGAATAAATT
TTTCGTTTAGGGTGATTTCCCACAAAGCACAGCGCGTAATTTGCATGCGC
TCTACCCCAGGCTCCTGTGCTAGACAAGAAGCCCGCGCATCCGGGCAAGG
GATGATGACGTCGTCCTTCAAGAGCG (SEQ ID NO: 752)
rattus_norvegicus
AGGAGTGTGAAGACCTGCCGCCATAATAAGACTCCAAAAGACAGTGAATT
TAACACTTACGGTGACTTCCCACAAAGCACAGCGTGTAATTTGCATGCGC
TCTAGCCCAGGCTCCAGCTCCGGACCAGAAGCCCGCGCATCCCGGCAAAG
GGTGATGACGTCGTCCTTCAAGCGCT (SEQ ID NO: 753)
dipodomys_ordii
AGGAAAGACTTCGCTGAGGCAGACTTTATAAGGCTCCCGCGCAGAAAGAA
ACTTTATAGTTATGGTGATTTCCCACAAGCCACTGCGTCATGCAAATAAA
GCAGGGTACGGCTTCCATGTACCTTAAGGTTTTTTTCTAGGCCGCGTACG
CTCTGCGTATTCAGCCACGTGACCCTGAGCCAGTGGTTGTTGGGAGCACG
TTGTGGACCTCTGCGTTTGGATTCC (SEQ ID NO: 754)
ictidomys_tridecemlineatus
GAAAGGGACTCCGCACAAGCAGAGTTTATAAGGCTCCCATCTGTACAGCC
ATTTCTCGGTCATGGTAACTACCCACAACACACAGCGATATGCAAATATA
GCAGAGCGTGTCTTCCCGCGCGCGCCTGGTCGTCTCGGCGCCGGCGCGCT
GCGTGGGGCGGAACTGTGACAGAGACCCTGCGATTCCTGGGAGCTGGCTG
ATGACATCAGTGTCTAACCTCC (SEQ ID NO: 755)
cavia_porcellus
GAGAAAGAAAGGCTCAAACCTAGCCTTATAAGGCTCCCAAATGTCGGTAT
ATTTTTTGGTTATGGTGACTTCCCACAATGCATAGCGATATGTAGATATT
GCCAGGAGTACCTCCCACTTCTGGTCCTGTCAGCTCTTTTCTAGGACGCG
CGCGCTGCAGGTTTCCAGCCTGTGATTGGGCCAGCAATTCCGGGAATGAA
TTGATGACGTCAGCGTTTGAATTCC (SEQ ID NO: 756)
ochotona_princeps
GGGGGAAGCTGGGCTCGATCAGCCTTTATAAAGCTCCAAAAACTCAAGAC
ATTTTTCTGTTACGGTGGCTTCCCACAGTACACAGCGACATGCAAATAGC
TTGCCAATGAATTCGCGGACCGCTTCCCGCCCCGGCGCAGGCGCGCGGAC
GCTGTCTCCCCTGGACGCGCGCTCGCGGTTCCCGGGAGCTGGCTGATGAC
GTTCGGTCTCC (SEQ ID NO: 757)
oryctolagus_cuniculus
GGGGAGAGGTGGATCCGAACAGACTTTATAAAGCTCCGAAAGCCCAAGGC
ATCTTTCCCTTACGGTAGCTTCCCACAAGACATAGCGACATGCAAATTTC
TTGAAGTATGCTTCAGACGCGCTTCTCGCCACAGCGCAAGCGCGCTGTGT
GCTGACGCGGGAACGGGCCAGGGCGCGGTTCCCGGGAGCGGGTTGATGAC
GTTAGATCTCC (SEQ ID NO: 758)
callithrix_jacchus
GAGGAAAAGTAGTCCCACAGACAACTTATAAGATTCCCATACCCTAAGAC
ATTTCACGATTATGGTGACTTCCCAGAAGACACAGCGACATGCAAATATT
GCAGGTCGTGTTTCGCCTGTCCCTCACAGTCGTCTTCCTGCCAGGGCGCA
CGCGCGCTGGGTTTCCCGCCAACTGACGCTGGGCTCGCGATTCCTTGGAG
CGGGTTGATGACGTCAGCGTTTGAATTCC (SEQ ID NO: 759)
chlorocebus_sabaeus
GGGGAAGGGTGGTCCCTTACAGAACTTATAAGATTCCCAAACTCAAAGAC
ATTTCACGTTTATGGTGACTTCCCAGAAGACATAGCGACATGCAAATATT
GCAGGGCGTCACACCCCTCTCCCTCACAGTCATCTTCCTGCCAGGGCGCA
CGCGCGCTGGGTGTTCTCGCGTAGTGACACTGGGCCCGCGATTCCTTGGA
GCGGGTTGATGACGTCAGCGTTCGAATTCC (SEQ ID NO: 760)
macaca_mulatta
GGGGAAGGGTGGTCCCACACAGAACTTATAAGATTCCCATACTCAAAGAC
ATTTCTCGTTTATGGTGACTTCCCAGAAGACACAGCGACATGCAAATATT
GTAGGGCGTCACACCCCTGTCCCTCACAGTCATCTTCCTGCCAGGGCGCA
CGCGCGCTGGGTGTTCCCGCGTAGTGACACTGGGCCCGCGATTCCTTGGA
GCGGGTTGATGACGTCAGCGTTCGAATTCC (SEQ ID NO: 761)
papio_anubis
GGGGAAAGGTGGTACCATACAGAACTTATAAGATTCCCATACTCAAAGAC
ATTTCACGATTATGGTGACTTCCCAGAAGACACAGCGACATGCAAATATT
GTAGGGCGTCACACCCCCTGTCCCTCACAGTCATCTTCCTGCCAGGGCGC
ACGCGCGCTGGGTGTTCCCGCGTAGTGACACTGGGCCCGCGATTCCTTGG
AGCGGGTTGATGACGTCAGCGTTCGAATTCC (SEQ ID NO: 762)
gorilla_gorilla
GGGAAAGGGTGGTCCCACACAGAACTTATAAGACTCCCATATCCAAAGAC
ATTTCACGGTTATGGTGATTTCCCAGAACACATAGCGACATGTAAATATT
GCAGGGCGCCACTCCCCAGTCCCTCACAGCCATCTTCCTGCCAGGGCGCA
CGCGCGCTGGGTGTTCCCGCCTAGTGACACTGGGCCCGCGATTCCTTGGA
GCGGGTTGATGACGTCAGCGTTCGAATTCC (SEQ ID NO: 763)
homo_sapiens
GGGAAAAAGTGGTCTCATACAGAACTTATAAGATTCCCAAATCCAAAGAC
ATTTCACGTTTATGGTGATTTCCCAGAACACATAGCGACATGCAAATATT
GCAGGGCGCCACTCCCCTGTCCCTCACAGCCATCTTCCTGCCAGGGCGCA
CGCGCGCTGGGTGTTCCCGCCTAGTGACACTGGGCCCGCGATTCCTTGGA
GCGGGTTGATGACGTCAGCGTTCGAATTCC (SEQ ID NO: 764)
pan_troglodytes
GGGAAAGGGTGGTGCCACACAGAACTTATAAGATTCCCATATGCAAAGAC
ATTTCACGTTTATGGTGATTTCCCAGAACACATAGCGACATGCAAATATT
GCAGGGCGCCACTCCCCTGTCCCTCACTGCCATCTTCCTGCCAGGGCGCA
CGCGCGCTGGGTGTTCCCGCCTAGTGACACTGGGCCCGCGATTCCTTGGA
GCGGGTTGATGACGTCAGCGTTCGAATTCC (SEQ ID NO: 765)
pongo_abelii
GAGAAAGGGTGGTCCCGTCCAGAACTTATAAGATTCCCATACCCAAAGAC
ATTTCACGTTTATGGTGACTTCCCAGAATGCATAGCGACATGCAAATATT
GCAGGGCGTCACTCCCCTGTCCCTCACAGCCATCTTCCTGCCAGGGCGCC
CGCGCGCTGGTGTTCCCGCCTAGTGACACTGGGCCCACGATTCCTTGGAG
CGGGTTGATGACGTCAGCGCTCGTATTCC (SEQ ID NO: 766)
nomascus_leucogenys
GGGGAAAAGTAGTAGACCTTATAAGATTCCCAAACCCAAAGACATTTCTC
GTTTATGGTGACTTCCCAGAAGACATAGCGACATGCAAATATTGCAGGGC
GCCACTCCCCTGTCCCTCACAGCCATCTTCCTGCCAGGGCGCACGCGCGC
TGGGTGTTCCCGCCTAGTGACACTCGGCCCGCGATTCCTTGGAGCGGGTT
GATGACGTCAGCGTTCGAATTCC (SEQ ID NO: 767)
tarsius_syrichta
GCGAGAGGGTGGGTCCACACAGAGCTTATAAGGTTCACAAGTAAAGATAT
TTCACGGTGACGGTGACTTCCCACAATACACTGCGACATGCAAATATAGC
CGGGCGTGCCTCCCCGATCCCGGAAGAGCGACTCCTAGCCAGTGCGCACG
CGCGCTGCGTGTTCGCGTCCTAGGTCGCTGGGCCCGCGGTTCCTGGGAGC
GGGTGGTGACGTCAGCGGCCCAGCTTC (SEQ ID NO: 768)
otolemur_garnettii
GCCTAAAAGGGCGCTTGCACAGAATTTATAAGGTTCCCAAACAGAGACAC
ATTTCATTATTATGGTGACTTCCCACAATGCACAGCGCCATGCAAATATG
CTAGGACGCCTCCCCCCGCTACCTTAAGGTCGTCAACTAACCAGTGCGCG
CGCGCACTGCGCGTTTCCCGCCGGTGACTCAATGCCCGCGTTTGGTGGGA
GCTAGTTGGTGACCTCAGTTCTGGAGGCTC (SEQ ID NO: 769)
tupaia_belangeri
GGGGGAAGCTGGGTCCACTGAGTTCTTATAAGGTTTCCAGTCCTAGAGCG
ATTTTACCATTGCGGTGATTTCCCAGCATCCGTAGCTACATGCAAATAGC
GCGGGGCGCGTCTCTCAGGTCCCTCCCCGCCCTCTCACTGTACGTACCCG
CGTCCTAGGGACGCCGCGCCCGGGGTTCCCGGACGTCAGCGTTCCGACGC
A (SEQ ID NO: 102)
ailuropoda_melanoleuca
AGGGAAAGCCGCGCCTGGGGCGGATTTATAAGGCTTCCATATCTAAAGGC
ATTTCACAGTCATGGTGACTTCCCACAATACATAGCAACATGCAAATATC
GCGGGGAGAACCTCCCCTGTCCCTTGTACGCGGCTTCTAAAGACGCACGC
GCGCTCTGTGTTCCCGCCCTGTGACTCTAGGCGGGCAATTCCTGGGACAG
TGTTCTGACGGGAACGTTCAGGCTCC (SEQ ID NO: 770)
mustela_putorius_furo
GGGAAAGGGTGGACCCACCGAGCATTTATAAGGCTCCCGCATCTAAAGAC
ATTTTACAGTTATGGTGACTTCCCACAACGCGTAGCAACATGCAAATATC
GTGGAGAGTACCGCCCCTGTCCCATGCACGCGTCTTCTCAGCAGCACGCA
CGCGCGCTGTGTTCCCGCCCTGTGACTCCAGGCGGGTATTTCCAGGGGCG
GGTTGCTGACAGGAACGTTCAGGCTTC (SEQ ID NO: 771)
canis_familiaris
GCAGCGCAGCCCTCTCGCCGCTTATAAAGTGCCGCCCGCACGGCCCTTCT
CGCTCACGGCGACTTCCCATAACACACAGCAGCATGCAAATACCGCGGGG
AGCCCCGCCCCGCCCCGGCCCCCGCACCGCCTCGGGACGCATGCGCCGGC
TCTCCGTTCCCGCCTTGGGCCGGCGGCGGGCGGGCGGGCGAGCGGGCGGG
AGCGGCTCCGGCGGGGACGAGCGGGCGCC (SEQ ID NO: 772)
felis_catus
GGGAAAGGGTGGCCCCGCCGAGCATTTATAAGACTCCCATACCTAAAGAC
ATTTCTCAGTTATGGTGATTTCCCACAACACACAGCAACATGCAAATATC
GAGGGGTGTACCGCCCCTGTCCTTTGTAGACGTCTTCTCTCCAGGACGCA
CGCGCGCTGTATTCCCGCCTTGTGACTCTAGGCGGGCGATTCCTGGGAGA
GGGTTGATGACGTCCAAGTTCTGGCTTC (SEQ ID NO: 773)
equus_caballus
GGGGGAAAACAGCCCATGGCTGCATTTATAAGACTCACAGATCTAAAGCC
ATTTCACGAATAGGGTGACTTCCCACAATACACAGCGACATGCAAACATA
GCGGGGCGTGCCTTTCCTGTACCCTGTGGGCATCTCTCCTGGACGCACGC
GCGCCGGGTGTTCCCGCGCTGTGACTCTAGGCAAGCGCTTCCTGGGAGAG
AGTTGATGACGGCAGCATTCGGGCTCC (SEQ ID NO: 774)
myotis_lucifugus
GGGAGAAGGAGGCGTAGAGGATATATAAGGCCCCCTTATGTGTAGTCCTT
TTACGGTTAGGGTGACTTCCCACAACGCATAGCGACATGCAAATTTGACG
GGCGTGCCTCCTCTGTCCCTGCGGGCAACTTCTCTCCTGGACGCGCGCGC
GCTGCGTGTTCCCGCCTTTTGACTCCAGCCGAGCGAATCCTGGGAGAGGG
CAGGTGACGTCAACAGTCAGGCTCG (SEQ ID NO: 775)
pteropus_vampyrus
GCGAGAAAAATTCTTCACGCAGAATATATAAGGATCCCATATCTGAAGAC
ATTTTACGATTACGGCGATTTCCCACAACACATAGCGACATGTAAATGTA
GTGGGGCATGCCTCCCCTGTCCCTTGTGGGCAGCTTCTCGCCAGAACGCA
CGCGCGGTGCGTGTTCCCGCCTTGTGACTAAGTTGGCGAGTCAGGGAGGA
GATTGATGACGTCAGCTCACCCGCTCC (SEQ ID NO: 776)
bos_taurus
GGCAAACACCGCACGCAAATAGCACTTATAATGTGCTCATACCTAGAGCC
ACTTTTCGGTTACGGTGACTTCTCAAAAAGACAGTGGAACATGCAAATAT
TACAGTGCGTCCCGCCCCTGGTAGGTCTACGCTAGGACGCACGCGCACTA
CGGTTCCCGCCTATAGACTGCGCTGGCGATTCCTGGGAGCGGACTGATGA
CGTCAGCGTTCGGGATCC (SEQ ID NO: 777)
ovis_aries
GGCGAACAATGCGCGCAAACAGCATTTATAATGAGCTCATACCTAAAGCC
ACTTTACGGTTACGGTGACTTCCCACAAGACATTGCGGCATGCAAATATT
TTAGTGCGTCCCGCCCCTGGTAGTTCCACGCTAGGACGCACACGCACTAC
GGTTCCCGCCTTTAGACTGCGCTGGCGATTCCAGGAGCGGACTGATGACG
TCAGCGTTGGGGCTCC (SEQ ID NO: 778)
tursiops_truncatus
GCCGAAAACCAGGCTCAAGCCACATTTATAAGGCTCCCAAATCTAAGTAC
ATTTGTCGGTTATGGTGACTTCCCGCACCACATTGCGACATGCAAATACT
GCGGAGCGTCCCTCCCCTGGCAACTCCTCGCTGGGACGCACGCGCGCTAC
GTGCTCCCGCCTTTTGACTGCGCCGGCGATACTTGGGAGAGGGTTGATGA
CGTCAGCGTTCTGGCTCC (SEQ ID NO: 779)
vicugna_pacos
GGGAAAGGGTGGGCTCACGCAGCCTTTATAAGACTCCCAAACTTAAAGAC
ATTTCTCGGTTATGGCGACTTCCCACAAGACATAGCGACATGCAAATACT
GCAGGGCGCCGACCCGGTCCTGTGCAGCCATCTTTCGGCTGGGACGCACG
CGCGCTGCGTGTTCCCGCCCTGTGACTGCGCCGGCGATTACTGGGAGAGG
ATTGATGACGTCAACGTTCGGGTTCC (SEQ ID NO: 780)
sus_scrofa
GTAGGAAAACTGCTTCTGTGAGCACTTATAAAACTCCCATAAGTAGAGAG
ATTTCATAGTTATGGTGATTTCCCATAAGACATTGCGACATGCAAATATT
GTGGCGCGTTCGTCCCCGTCCGGTGCAGGCAGCTTCGCTCCAGGACGCAC
GCGCAATACATGTTCCCGCCTTGAGACTGCGCCGGCAGATTCCTAGGAAG
TGGTTGATGACGTCGATGTTAGGGATCC (SEQ ID NO: 781)
erinaceus_europaeus
GCCTAAACCGGCTCTTTCGACAGACTTATAAGGACCTCTTATCTTAGGAC
ATTTTTTTGTTAGGGTAACTTCCCACGATGCATAGCGATATGTAAATATG
GCGCCGCGAGTCTCTCCTAGGCGTCTCCCCAGGACGCAGGCGCACTGCTT
GTTCCCGCGTTAACATTGCTGATTCTGGGAGACTGCTGATGACGTCAGCG
TCCAGTCTAC (SEQ ID NO: 782)
choloepus_hoffmanni
AGAAAAAAATAGTTTATGCTGGATTTATAAGATTCCCAAATCTAAAGCCA
TTTCACAGTTACGGTGATTCCCCACTACACACGGCGATATGCAAATATAG
CGGAAGTGTTCCTGAGGCGTGGTAAAGCGCGCGCGCGCTGAGAGTTCCCG
CCCTGTGGTGCTGGGCTGGAGATGCCTGAGAACTGGCTGATGACGGCAAC
GTTCGGGCTCC (SEQ ID NO: 783)
dasypus_novemcinctus
AAAGCGATAGTTTTTTAAACTGGACTTATAAGGCACCCATATCTACGTAT
ATTTCATGGTTAGGGTGATTTCCCACAACACATAGCGAAATGCAAATATT
GGAGGGCGCTGAGGCGTGGTCGGGCGCAAGCGCGCTGCGACTTCCCGCCT
TTCGGCCCTAGGCCCCAGATTCCTGGGAGCTGGATGATGACGTTGACGTT
CGGATACC (SEQ ID NO: 784)
loxodonta_africana
GGGAAGGAACAAATTCGTCAGGATTTATAAGACTCTCAGAGCTGTAGACA
TTTCACAGTTAGGGCGATGTCCCACAATACATAGCAACATGCAAATATTC
TAGGAGGCCAGCCTCCCCGTCCGCGTGGTCATCTTCTCGCTAGGGCGCAC
GCCCGCTGCGTGTTCCCGCTCTGTGACCAGGCAGGCGATTCCTGAGAACC
GCTTGGTGACGTCAGTGTTCTGGCTCC (SEQ ID NO: 785)
procavia_capensis
AGGGTAAATCGGCGCTGCTCAGCATTTAAAAGAATCCCAAATGTGTCGC
CATTTTACGCTTAGGGTGATATCCCACAAGACACAGCGACATGCAAATAT
CGTGAGTCTCTGTTTCCCTGTCCACGAGGGCGTCCTCTCGCTGGGGCGCA
CGCGCGGTGTGTGTGCCCCCGTTGTGTGTTCCCGCGATTCCAAAGAACTG
GTTGATAACGTTAGACTTCCGGCTGC (SEQ ID NO: 786)

### B. Methods

In some embodiments, the presently disclosed subject matter also provides a method of altering expression of one or more gene products in a eukaryotic cell, wherein the cell comprises a DNA molecule encoding the one or more gene products, the method comprising introducing into the cell a modified non-naturally occurring CRISPR system previously described in WO2015/195621 (herein incorporated by reference in its entirety). Such a modification uses certain gRNAs that target retinal degeneration-related genes, such as, but not limited, to LCA10 CEP290 gene, rhodopsin, Dual Leucine Zipper Kinase (DLK), Leucine Zipper Kinase (LZK), JNK1-3, MKK4, MKK7, ATF2, JUN, MEF2A, SOX11, or PUMA. In some embodiments, the method comprising introducing into the cell a composition comprising (a) a non-naturally occurring nuclease system (e.g., CRISPR) comprising one or more vectors comprising: i) a promoter (e.g., bidirectional H1 promoter) operably linked to at least one nucleotide sequence encoding a nuclease system guide RNA (gRNA), wherein the gRNA hybridizes with a target sequence of a DNA molecule in a cell of the subject, and wherein the DNA molecule encodes one or more gene products expressed in the cell; and ii) a regulatory element operable in a cell operably linked to a nucleotide sequence encoding a genome-targeted nuclease (e.g., Cas9 protein), wherein components (i) and (ii) are located on the same or different vectors of the system, wherein the gRNA targets and hybridizes with the target sequence and the nuclease cleaves the DNA molecule to alter expression of the one or more gene products. In some embodiments, the system is packaged into a single adeno-associated virus (AAV) particle. In some embodiments, the adeno-associated virus (AAV) may comprise any of the 51 human adenovirus serotypes (e.g., serotypes 2, 5, or 35). In some embodiments, the system inactivates one or more gene products. In some embodiments, the nuclease system excises at least one gene mutation. In some embodiments, the promoter comprises: a) control elements that provide for transcription in one direction of at least one nucleotide sequence encoding a gRNA; and b) control elements that provide for transcription in the opposite direction of a nucleotide sequence encoding a genome-targeted nuclease. In some embodiments, the Cas9 protein is codon optimized for expression in the cell. In some embodiments, the promoter is operably linked to at least one, two, three, four, five, six, seven, eight, nine, or ten gRNA. In some embodiments, the target sequence is a mutation in the CEP290 gene (e.g., LCA10 CEP290 gene). In some embodiments, the target sequence for CEP290 is selected from the group consisting of the nucleotide sequences set forth in SEQ ID NO: 1-109, 164-356, 735-738, or combinations thereof. In some embodiments, the target sequence comprises SEQ ID NOs: 1, 2, 3, and 4 operably linked. In some embodiments, the vector comprises the nucleotide sequence set forth in SEQ ID NO: 110. In some embodiments, the one or more gene products are rhodopsin. In some embodiments, the target sequence is a mutation in the rhodopsin gene. In some embodiments, the target sequence is a mutation at R135 of the rhodopsin gene (e.g., R135G, R135W, R135L). In some embodiments, the target sequence for rhodopsin R135 is selected from the group consisting of the nucleotide sequences set forth in SEQ ID NO: 111-126, or combinations thereof. In some embodiments, the gRNA sequence for rhodopsin R135 is selected from the group consisting of the nucleotide sequences set forth in SEQ ID NO: 127-142, or combinations thereof. In some embodiments, the one or more gene products are Dual Leucine Zipper Kinase (DLK), Leucine Zipper Kinase (LZK), JNK1-3, MKK4, MKK7, ATF2, JUN, MEF2A, SOX11, or PUMA, or combinations thereof. In some embodiments, the mutation targets of glaucoma include, but not limited to, OPTN, TBK1, TMCO1, PMM2, GMDS, GAS7, FNDC3B, TXNRD2, ATXN2, CAV1/CAV2, p16INK4a, SIX6, ABCA1, AFAP1 and CDKN2B-AS. In some embodiments, the target sequence for glaucoma is selected from the group consisting of the nucleotide sequences set forth in SEQ ID NO: 143-163, or combinations thereof.

In some embodiments, the presently disclosed subject matter also provides a method of altering expression of one or more gene products in a cell, wherein the cell comprises a DNA molecule encoding the one or more gene products, the method comprising introducing into the cell a non-naturally occurring CRISPR system comprising one or more vectors comprising: a) an H1 promoter operably linked to at least one nucleotide sequence encoding a CRISPR system guide RNA (gRNA), wherein the gRNA hybridizes with a target sequence of the DNA molecule; and b) a regulatory element operable in the cell operably linked to a nucleotide sequence encoding a Cas9 protein, wherein components (a) and (b) are located on the same or different vectors of the system, wherein the gRNA targets and hybridizes with the target sequence and the Cas9 protein cleaves the DNA molecule to alter expression of the one or more gene products.

In some embodiments, the presently disclosed subject matter also provides a method of altering expression of one or more gene products in a eukaryotic cell, wherein the cell comprises a DNA molecule encoding the one or more gene products, the method comprising introducing into the cell a non-naturally occurring CRISPR system comprising one or more vectors comprising: a) an H1 promoter operably linked to at least one nucleotide sequence encoding a CRISPR system guide RNA (gRNA), wherein the gRNA hybridizes with a target sequence of the DNA molecule; and b) a regulatory element operable in the eukaryotic cell operably linked to a nucleotide sequence encoding a Type-II Cas9 protein, wherein components (a) and (b) are located on the same or different vectors of the system, whereby the gRNA targets and hybridizes with the target sequence and the Cas9 protein cleaves the DNA molecule, and whereby expression of the one or more gene products is altered. In one aspect, the target sequence can be a target sequence that starts with any nucleotide, for example, N₂₀NGG. In some embodiments, the target sequence comprises the nucleotide sequence AN₁₉NGG. In some embodiments, the target sequence comprises the nucleotide sequence GN₁₉NGG. In some embodiments, the target sequence comprises the nucleotide sequence CN₁₉NGG. In some embodiments, the target sequence comprises the nucleotide sequence TN₁₉NGG. In some embodiments, the target sequence comprises the nucleotide sequence AN₁₉NGG or GN₁₉NGG. In another aspect, the Cas9 protein is codon optimized for expression in the cell. In yet another aspect, the Cas9 protein is codon optimized for expression in the eukaryotic cell. In a further aspect, the eukaryotic cell is a mammalian or human cell. In another aspect, the expression of the one or more gene products is decreased.

The presently disclosed subject matter also provides a method of altering expression of one or more gene products in a eukaryotic cell, wherein the cell comprises a DNA molecule encoding the one or more gene products, the method comprising introducing into the cell a non-naturally occurring CRISPR system comprising a vector comprising a bidirectional H1 promoter, wherein the bidirectional H1 promoter comprises: a) control elements that provide for transcription in one direction of at least one nucleotide sequence encoding a CRISPR system guide RNA (gRNA), wherein the gRNA hybridizes with a target sequence of the DNA molecule; and b) control elements that provide for transcription in the opposite direction of a nucleotide sequence encoding a Type-II Cas9 protein, whereby the gRNA targets and hybridizes with the target sequence and the Cas9 protein cleaves the DNA molecule, and whereby expression of the one or more gene products is altered. In one aspect, the target sequence can be a target sequence that starts with any nucleotide, for example, N₂₀NGG. In some embodiments, the target sequence comprises the nucleotide sequence AN₁₉NGG. In some embodiments, the target sequence comprises the nucleotide sequence GN₁₉NGG. In some embodiments, the target sequence comprises the nucleotide sequence CN₁₉NGG. In some embodiments, the target sequence comprises the nucleotide sequence TN₁₉NGG. In another aspect, the target sequence comprises the nucleotide sequence AN₁₉NGG or GN₁₉NGG. In another aspect, the Cas9 protein is codon optimized for expression in the cell. In yet another aspect, the Cas9 protein is codon optimized for expression in the eukaryotic cell. In a further aspect, the eukaryotic cell is a mammalian or human cell. In another aspect, the expression of the one or more gene products is decreased.

In some aspects, the presently disclosed subject matter provides methods comprising delivering one or more polynucleotides, such as or one or more vectors as described herein, one or more transcripts thereof, and/or one or proteins transcribed therefrom, to a host cell. In some aspects, the presently disclosed subject matter further provides cells produced by such methods, and organisms (such as animals, plants, or fungi) comprising or produced from such cells. In some embodiments, a CRISPR enzyme in combination with (and optionally complexed with) a guide sequence is delivered to a cell. Conventional viral and non-viral based gene transfer methods can be used to introduce nucleic acids in mammalian cells or target tissues Such methods can be used to administer nucleic acids encoding components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson (1992) Science 256:808-813; Nabel and Felgner (1993) TIBTECH 11:211-217; Mitani and Caskey (1993) TIBTECH 11:162-166; Dillon (1993) TIBTECH 11:167-175; Miller (1992) Nature 357.455-460; Van Brunt (1998) Biotechnology 6(10): 1149-1154; Vigne (1995) Restorative Neurology and Neuroscience 8:35-36; Kremer and Perricaudet (1995) British Medical Bulletin 51(1l):31-44; Haddada et al. (1995) Current Topics in Microbiology and Immunology. Doerfler and Bohm (eds); and Yu et al. (1994) Gene Therapy 1:13-26.

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (e.g., Crystal (1995) Science 270:404-410; Blaese et al. (1995) Cancer Gene Ther. 2:291-297: Behr et al. (1994) Bioconjugate Chem. 5:382-389; Remy et al. (1994) Bioconjugate Chem. 5:647-654; Gao et al. (1995) Gene Therapy 2:710-722; Ahmad et al. (1992) Cancer Res. 52:4817-4820; U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (e.g., Buchscher et al. (1992) J. Virol. 66:2731-2739; Johann et al. (1992) J. Virol. 66:1635-1640; Sommnerfelt et al. (1990) J. Virol. 176:58-59; Wilson et al. (1989) J. Virol. 63:2374-2378; Miller et al. (1991) J. Virol. 65:2220-2224; PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (e.g., West et al. (1987) Virology 160:38-47; U.S. Pat. No. 4,797,368; WO 93/24641; Kotin (1994) Human Gene Therapy 5:793-801; Muzyczka (1994) J. Clin. Invest. 94:1351. Construction of recombinant AAV vectors are described in a number of publications, including U.S. Pat. No. 5,173,414; Tratschin et al. (1985) Mol. Cell. Biol. 5:3251-3260; Tratschin et al. (1984) Mol. Cell. Biol. 4:2072-2081; Hermonat and Muzyczka (1984) Proc. Natl. Acad. Sci. U.S.A. 81:6466-6470; and Samulski et al. (1989) J. Virol. 63:03822-3828.

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panel, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CIO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-MeI 1-48, MC-38, MCF-7, MCF-10A, MDA-MB-231, MDA-MB-468, MDA-MB-435, MDCK II, MDCK II, MOR/0.2R, MONO-MAC 6, MTD-1 A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

In some embodiments, one or more vectors described herein are used to produce a non-human transgenic animal. In some embodiments, the transgenic animal is a mammal, such as a mouse, rat, or rabbit. In certain embodiments, the organism or subject is a plant. Methods for producing transgenic animals are known in the art, and generally begin with a method of cell transfection, such as described herein.

In one aspect, the presently disclosed subject matter provides for methods of modifying a target polynucleotide in a eukaryotic cell, which may be in vivo, ex vivo or in vitro. In some embodiments, the method comprises sampling a cell or population of cells from a human or non-human animal, and modifying the cell or cells. Culturing may occur at any stage ex vivo. The cell or cells may even be re-introduced into the non-human animal.

In one aspect, the presently disclosed subject matter provides for methods of modifying a target polynucleotide in a eukaryotic cell. In some embodiments, the method comprises allowing a CRISPR complex to bind to the target polynucleotide to effect cleavage of the target polynucleotide thereby modifying the target polynucleotide, wherein the CRISPR complex comprises a CRISPR enzyme complexed with a guide sequence hybridized to a target sequence within the target polynucleotide.

In one aspect, the presently disclosed subject matter provides a method of modifying expression of a polynucleotide in a eukaryotic cell. In some embodiments, the method comprises allowing a CRISPR complex to bind to the polynucleotide such that the binding results in increased or decreased expression of the polynucleotide; wherein the CRISPR complex comprises a CRISPR enzyme complexed with a guide sequence hybridized to a target sequence within the polynucleotide.

In one aspect, the presently disclosed subject matter provides methods for using one or more elements of a CRISPR system. The CRISPR complex of the presently disclosed subject matter provides an effective means for modifying a target polynucleotide. The CRISPR complex of the presently disclosed subject matter has a wide variety of utility including modifying (e.g., deleting, inserting, translocating, inactivating, activating) a target polynucleotide in a multiplicity of cell types. As such the CRISPR complex of the presently disclosed subject matter has a broad spectrum of applications in, e.g., gene therapy, drug screening, disease diagnosis, and prognosis. An exemplary CRISPR complex comprises a CRISPR enzyme complexed with a guide sequence hybridized to a target sequence within the target polynucleotide.

The target polynucleotide of a CRISPR complex can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA). Without wishing to be bound by theory, it is believed that the target sequence should be associated with a PAM (protospacer adjacent motif); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence). Examples of PAM sequences are given in the examples section below, and the skilled person will be able to identify further PAM sequences for use with a given CRISPR enzyme.

Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

Embodiments of the presently disclosed subject matter also relate to methods and compositions related to knocking out genes, amplifying genes and repairing particular mutations associated with retinal disorders (Robert D. Wells, Tetsuo Ashizawa, Genetic Instabilities and Neurological Diseases, Second Edition, Academic Press, Oct. 13, 2011-Medical). Specific aspects of tandem repeat sequences have been found to be responsible for more than twenty human diseases (McIvor et al. (2010) RNA Biol. 7(5):551-8). The CRISPR system may be harnessed to correct these defects of genomic instability.

In yet another aspect of the presently disclosed subject matter, the CRISPR system may be used to correct retinal defects that arise from several genetic mutations further described in Traboulsi, ed. (2012) Genetic Diseases of the Eye, Second Edition, Oxford University Press.

### II. Methods for Treating Retinal Degenerations

The presently disclosed subject matter also provides methods for treating retinal degenerations, such as LCA, ADRP, or glaucoma. In some embodiments, the presently disclosed subject matter provides method for treating a retinal degeneration in a subject (e.g., human) in need thereof. The method comprises the steps of: (a) providing a non-naturally occurring nuclease system (e.g., CRISPR) comprising one or more vectors comprising: i) a promoter (e.g., bidirectional H1 promoter) operably linked to at least one nucleotide sequence encoding a nuclease system guide RNA (gRNA), wherein the gRNA hybridizes with a target sequence of a DNA molecule in a cell (e.g., retinal photoreceptor or ganglion cell) of the subject, and wherein the DNA molecule encodes one or more gene products expressed in the cell; and ii) a regulatory element operable in a cell operably linked to a nucleotide sequence encoding a genome-targeted nuclease (e.g., Cas9), wherein components (i) and (ii) are located on the same or different vectors of the system, wherein the gRNA targets and hybridizes with the target sequence and the nuclease cleaves the DNA molecule to alter expression or inactivates of the one or more gene products; and (b) administering to the retinal area of the subject a therapeutically effective amount of the system. In some embodiments, the system is packaged into a single adeno-associated virus (AAV) particle (e.g., AAV, AAV2, AAV9, and the like). In some embodiments, the nuclease system excises at least one gene mutation. In some embodiments, the H1 promoter comprises a) control elements that provide for transcription in one direction of at least one nucleotide sequence encoding a gRNA; and b) control elements that provide for transcription in the opposite direction of a nucleotide sequence encoding a genome-targeted nuclease. In some embodiments, the promoter is operably linked to at least one, two, three, four, five, six, seven, eight, nine, or ten gRNA. In some embodiments, the retinal degeneration is selected from the group consisting of LCA1, 2, 3, 4, 5, 6, 7, 8, 9, 10, 11, 12, 13, 14, 15, 16, 17, and 18. In some embodiments, the retinal degeneration is LCA10. In some embodiments of treating LCA, the target sequence is selected from LCA10 CEP290 gene. In some embodiments of treating LCA, the target sequences is located in the LCA10 CEP290 gene and selected from the group consisting of the nucleotide sequences set forth in SEQ ID NO: 1-109, 164-356, 735-738, or combinations thereof (e.g., SEQ ID NOs: 1, 2, 3, and 4 operably linked). In some embodiments of treating LCA, the vector comprises the nucleotide sequence set forth in SEQ ID NO: 110. In some embodiments, the retinal degeneration is an ADRP. In some embodiments of treating ADRP, the target sequence is a mutation in the rhodopsin gene. In some embodiments of treating ADRP, the target sequence is a mutation at R135 of the rhodopsin gene. In some embodiments, the mutation at R135 is selected from the group consisting of R135G, R135W, R135L. In some embodiments of treating ADRP, the target sequence is selected from the group consisting of the nucleotide sequences set forth in SEQ ID NO: 111-126, or combinations thereof. In some embodiments of treating ADRP, the gRNA sequence is selected from the group consisting of the nucleotide sequences set forth in SEQ ID NO: 127-142, or combinations thereof. In some embodiments, the retinal degeneration is glaucoma. In some embodiments of treating glaucoma, the one or more gene products are Dual Leucine Zipper Kinase (DLK), Leucine Zipper Kinase (LZK), JNK1-3, MKK4 and MKK7, ATF2, JUN, MEF2A, SOX11, or PUMA, or combinations thereof. In some embodiments of treating glaucoma, the one or more gene products are identified using the RNA-based screens described in Example 4 infra. In some embodiments, the mutation targets of glaucoma include, but not limited to, OPTN, TBK1, TMCO1, PMM2, GMDS, GAS7, FNDC3B, TXNRD2, ATXN2, CAV1/CAV2, p16INK4a, SIX6, ABCA1, AFAP1 and CDKN2B-AS. In some embodiments of treating glaucoma, the target sequence is selected from the group consisting of the nucleotide sequences set forth in SEQ ID NO: 143-163, or combinations thereof. In some embodiments, administering to the subject occurs by implantation, injection (e.g., subretinal), or virally.

The CRISPR system may be used facilitate targeted genome editing in eukaryotic cells, including mammalian cells, such as human cells. To facilitate genome editing, the cell to be modified is co-transfected with an expression vector encoding Cas9 or the Cas9 protein, DNA, or RNA itself, along with a guide-RNA molecule itself, or an expression vector comprising a nucleic acid molecule encoding the guide-RNA molecule. For example, in certain embodiments, the introduction of Cas9 can be done by transfecting in Cas9 as a protein, RNA, DNA, or expression vector comprising a nucleic acid that encodes Cas9. In certain embodiments, the guide DNA can itself be administered directly as an RNA molecule (gRNA), DNA molecule, or as expression vector comprising a nucleic acid that encodes the gRNA.

By “retinal degeneration” is meant a disease, disorder, or condition (including an optic neuropathy) associated with degeneration or dysfunction of neurons or other neural cells, such as retinal ganglion or photoreceptor cells. A retinal degeneration can be any disease, disorder, or condition in which decreased function or dysfunction of neurons, or loss or neurons or other neural cells, can occur.

Such diseases, disorders, or conditions include, but are not limited to, glaucoma, amyotrophic lateral sclerosis (ALS), trigeminal neuralgia, glossopharyngeal neuralgia, Bell's Palsy, myasthenia gravis, muscular dystrophy, progressive muscular atrophy, primary lateral sclerosis (PLS), pseudobulbar palsy, progressive bulbar palsy, spinal muscular atrophy, inherited muscular atrophy, invertebrate disk syndromes, cervical spondylosis, plexus disorders, thoracic outlet destruction syndromes, peripheral neuropathies, porphyria, Alzheimer's disease, Huntington's disease, Parkinson's disease, Parkinson's-plus diseases, multiple system atrophy, progressive supranuclear palsy, corticobasal degeneration, dementia with Lewy bodies, frontotemporal dementia, demyelinating diseases, Guillain-Barre syndrome, multiple sclerosis, Charcot-Marie-Tooth disease, prion diseases, Creutzfeldt-Jakob disease, Gerstmann-Straussler-Scheinker syndrome (GSS), fatal familial insomnia (FFI), bovine spongiform encephalopathy (BSE), Pick's disease, epilepsy, and AIDS demential complex.

Other diseases, disorders, or conditions include, but not limited to, Alexander's disease, Alper's disease, ataxia telangiectasia, Batten disease (also known as Spielmeyer-Vogt-Sjogren-Batten disease), Canavan disease, Cockayne syndrome, diabetic neuropathy, frontotemporal lobar degeneration, HIV-associated dementia, Kennedy's disease, Krabbe's disease, neuroborreliosis, Machado-Joseph disease (Spinocerebellar ataxia type 3), wet or dry macular degeneration, Niemann Pick disease, Pelizaeus-Merzbacher Disease, photoreceptor degenerative diseases, such as retinitis pigmentosa and associated diseases, Refsum's disease, Sandhoffs disease, Schilder's disease, subacute combined degeneration of spinal cord secondary to pernicious anemia, Spielmeyer-Vogt-Sjogren-Batten disease (also known as Batten disease), spinocerebellar ataxia (multiple types with varying characteristics), Steele-Richardson-Olszewski disease, tabes dorsalis, lattice dystrophy, retinitis pigmentosa, age-related macular degeneration (AMD), photoreceptor degeneration associated with wet or dry AMD, other retinal degeneration such as retinitis pigmentosa (RP), optic nerve drusen, optic neuropathy, and optic neuritis, such as optic neuritis resulting from multiple sclerosis.

Non-limiting examples of different types of glaucoma that can be prevented or treated according to the presently disclosed subject matter include primary glaucoma (also known as primary open-angle glaucoma, chronic open-angle glaucoma, chronic simple glaucoma, and glaucoma simplex), low-tension glaucoma, primary angle-closure glaucoma (also known as primary closed-angle glaucoma, narrow-angle glaucoma, pupil-block glaucoma, and acute congestive glaucoma), acute angle-closure glaucoma, chronic angle-closure glaucoma, intermittent angle-closure glaucoma, chronic open-angle closure glaucoma, pigmentary glaucoma, exfoliation glaucoma (also known as pseudoexfoliative glaucoma or glaucoma capsulare), developmental glaucoma (e.g., primary congenital glaucoma and infantile glaucoma), secondary glaucoma (e.g., inflammatory glaucoma (e.g., uveitis and Fuchs heterochromic iridocyclitis)), phacogenic glaucoma (e.g., angle-closure glaucoma with mature cataract, phacoanaphylactic glaucoma secondary to rupture of lens capsule, phacolytic glaucoma due to phacotoxic meshwork blockage, and subluxation of lens), glaucoma secondary to intraocular hemorrhage (e.g., hyphema and hemolytic glaucoma, also known as erythroclastic glaucoma), traumatic glaucoma (e.g., angle recession glaucoma, traumatic recession on anterior chamber angle, postsurgical glaucoma, aphakic pupillary block, and ciliary block glaucoma), neovascular glaucoma, drug-induced glaucoma (e.g., corticosteroid induced glaucoma and alpha-chymotrypsin glaucoma), toxic glaucoma, and glaucoma associated with intraocular tumors, retinal detachments, severe chemical burns of the eye, and iris atrophy. In certain embodiments, the neurodegenerative disease, disorder, or condition is a disease, disorder, or condition that is not associated with excessive angiogenesis, for example, a glaucoma that is not neovascular glaucoma. In some embodiments, the mutation targets of glaucoma include, but not limited to, OPTN, TBK1, TMCO1, PMM2, GMDS, GAS7, FNDC3B, TXNRD2, ATXN2, CAV1/CAV2, p16INK4a, SIX6, ABCA1, AFAP1 and CDKN2B-AS.

As used herein, the term “disorder” in general refers to any condition that would benefit from treatment with a compound against one of the identified targets, or pathways, including any disease, disorder, or condition that can be treated by an effective amount of a compound against one of the identified targets, or pathways, or a pharmaceutically acceptable salt thereof.

As used herein, the term “treating” can include reversing, alleviating, inhibiting the progression of, preventing or reducing the likelihood of the disease, disorder, or condition to which such term applies, or one or more symptoms or manifestations of such disease, disorder or condition (e.g., a disease or disorder that causes dysfunction and/or death of retinal ganglion or photoreceptor cells). In some embodiments, the treatment reduces the dysfunction and/or death of retinal ganglion or photoreceptor cells. For example, the treatment can reduce the dysfunction and/or death of retinal ganglion or photoreceptor cells by at least 5%, 10%, 15%, 20%, 25%, 30%, 33%, 35%, 40%, 45%, 50%, 55%, 60%, 66%, 70%, 75%, 80%, 85%, 90%, 91%, 92%, 93%, 94%, 95%, 96%, 97%, 98%, 99% or more as compared to the dysfunction and/or death of retinal ganglion or photoreceptor cells in a subject before undergoing treatment or in a subject who does not undergo treatment. In some embodiments, the treatment completely inhibits dysfunction and/or death of retinal photoreceptor or ganglion cells in the subject. As used herein, a “retinal ganglion or photoreceptor cell” is a specialized type of neuron found in the retina that is capable of phototransduction. In some embodiments, at least one gene product is rhodopsin.

In some embodiments, the system is packaged into a single adeno-associated virus (AAV) particle before administering to the subject. In some embodiments, administering to the subject occurs by subretinal injection. The treatment, administration, or therapy can be consecutive or intermittent. Consecutive treatment, administration, or therapy refers to treatment on at least a daily basis without interruption in treatment by one or more days. Intermittent treatment or administration, or treatment or administration in an intermittent fashion, refers to treatment that is not consecutive, but rather cyclic in nature. Treatment according to the presently disclosed methods can result in complete relief or cure from a disease, disorder, or condition, or partial amelioration of one or more symptoms of the disease, disease, or condition, and can be temporary or permanent. The term “treatment” also is intended to encompass prophylaxis, therapy and cure.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

The term “inhibit” or “inhibits” means to decrease, suppress, attenuate, diminish, arrest, or stabilize the development or progression of a disease, disorder, or condition, the activity of a biological pathway, or a biological activity, e.g., by at least 10%, 20%, 30%, 40%, 50%, 60%, 70%, 80%, 90%, 95%, 98%, 99%, or even 100% compared to an untreated control subject, cell, biological pathway, or biological activity or compared to the target, in a subject before the subject is treated. By the term “decrease” is meant to inhibit, suppress, attenuate, diminish, arrest, or stabilize a symptom of a retinal disease, disorder, or condition. It will be appreciated that, although not precluded, treating a disease, disorder or condition does not require that the disease, disorder, condition or symptoms associated therewith be completely eliminated.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

“Pharmaceutically-acceptable salts” refers to the relatively non-toxic, inorganic and organic acid addition salts of compounds.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The terms “subject” and “patient” are used interchangeably herein. The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease.

The term “therapeutic effect” refers to a local or systemic effect in animals, particularly mammals, and more particularly humans, caused by a pharmacologically active substance.

The terms “therapeutically-effective amount” and “effective amount” as used herein means that amount of a composition of the present invention which is effective for producing some desired therapeutic effect in at least a sub-population of cells in an animal at a reasonable benefit/risk ratio applicable to any medical treatment.

### III. General Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXEMPLIFICATIONS

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Methods

Human embryonic kidney (HEK) cell line 293T (Life Technologies, Grand Island, NY) was maintained at 37° C. with 5% CO₂/20% O₂ in Dulbecco's modified Eagle's Medium (DMEM) (Invitrogen) supplemented with 10% fetal bovine serum (Gibco, Life Technologies, Grand Island, NY) and 2 mM GlutaMAX (Invitrogen).

gRNAs targeting Rhodopsin (see Ranganathan, V and Zack, D J. grID: A CRISPR guide RNA Database and Resource for Gene-Editing. Submitted (2015)) was generated by overlapping oligos that were annealed and amplified by PCR using two-step amplification Phusion Flash DNA polymerase (Thermo Fisher Scientific, Rockford, IL), and subsequently purified using Zymo DNA clean and concentrator columns. The purified PCR products were then resuspended in H₂O and quantitated using a NanoDrop 1000 (Thermo Fisher Scientific). The gRNA-expressing constructs were generated using the Gibson Assembly (New England Biolabs, Ipswich, MA) (Gibson et al. Nature Methods 6:343-345(2009)) with slight modifications. The total reaction volume was reduced from 20 μl to 2 μl. Clones were verified by Sanger sequencing.

HEK293 cells were co-transfected with Cas9 (unmodified, or cell-cycle regulated versions) and the gRNA construct targeting rhodopsin. 48 hrs post transfection, genomic DNA was harvested and the sequence surrounding the target cut sites were amplified according to the primers listed in the Appendix. The PCR products were then purified and quantitated before performing the T7 Endo I assay. Briefly, 200 ng of PCR product was denatured and then slowly re-annealed to allow for the formation of heteroduplexes, T7 Endonuclease I was added to the PCR products and incubated at 37° C. for 25 minutes to cleave heteroduplexes, the reaction was quenched in loading dye, and finally, the reaction was run on a 6% TBE PAGE gel to resolve the products. The gel was stained with SYBR-Gold, visualized, and quantitated using ImageJ. NHEJ frequencies were calculated using the binomial-derived equation:

$$\% gene modification = 1 - \sqrt{1 - \frac{( a + b )}{( a + b + c )}} \times 100 ;$$

where the values of “a” and “b” are equal to the integrated area of the cleaved fragments after background subtraction and “c” is equal to the integrated area of the un-cleaved PCR product after background subtraction.

### Example 1

While still in its infancy, the CRISPR system has revolutionized genome-editing technology, transformed biological research, and ushered in a new era for genetic medicine. Directed against sequences in the human genome, CRISPR-based editing systems can be customized to disrupt any gene or regulatory element, or to delete and replace genomic DNA sequences in a highly-specific fashion. And although numerous studies across the world have demonstrated that disease mutations can be efficiently targeted in vitro, the development of CRISPR-based therapeutics for in vivo use has been significantly hampered by delivery constraints.

Adeno-associated virus (AAV) vectors are the most frequently used viral vectors in gene therapy with several attractive features: the virus is non-pathogenic, it infects both dividing and non-dividing cells, expression can persist for long periods of time, as well as a noteworthy history of safety, efficacy, and a general lack of toxicity in clinical trials. Additionally, combinations of variant AAV serotypes are effective at targeting specific cell types. While AAV vectors provide a safe means of delivering therapeutic CRISPR components, there is one major technical obstacle that limits their utility—their size. Wild type AAV genomes are ˜4.7 kb in length and recombinant viruses can package up to 5.0 kb. This packaging capacity defines the upper limit of the DNA that can be used for a single viral vector.

The CRISPR/Cas9 system is composed of the nuclease (Cas9) and a guide RNA (gRNA), which serves to direct the nuclease to a specific region in a genome. The most commonly used Cas9 protein is from S. pyogenes (SpCas9), which is encoded by a 4104 bp open reading frame. It has been assumed that due to the large size of SpCas9, delivery of both CRISPR components, including promoters and terminator sequences necessary for expression, is limited by the AAV packaging capacity. With standard promoter elements in place, the SpCas9 and gRNA cassettes exceed the packaging capacity of AAV by a significant margin.

A solution to the packaging capacity problem was disclosed in WO2015/195621 (herein incorporated by reference in its entirety) that greatly expands the potential range of applications for therapeutic CRISPRs. At the core of this new approach to CRISPR delivery is a compact bidirectional promoter that is highly active in human cells. The H1 promoter is a remarkably unique genetic element that is recognized by RNA polymerases Pol II and Pol III. Introduced into an AAV vector, the H1 promoter can efficiently express both Cas9 and gRNA. Because the optimized H1 promoter is only ˜230 bp in length, the use of this cassette allows the packaging of SpCas9 and multiple hybrid gRNAs in a single recombinant AAV.

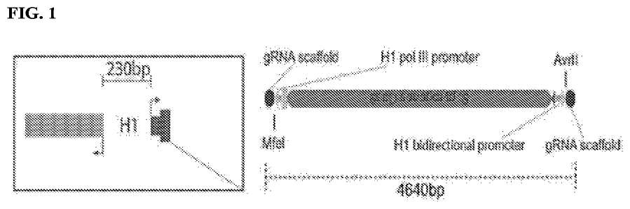

A complete “all-in-one” AAV vector that includes SpCas9, a short poly-A (SPA) sequence, two separately customizable gRNA scaffolds and the H1 promoter element is ˜4640 kb. This is nearly the size of the wild type AAV genome, and still well below the maximum packaging capacity of 5.2 kb. Any of the Cas9 genes can be incorporated into this basic structure. This platform thus provides the capability to target many more genomic sites and mutations, in vivo, than any existing technology (FIG. 1).

Another viable approach for clinical delivery, highlighted by the Cas9 from S. aureus (SaCas9), is the use of smaller Cas9 orthologs that can be packaged into AAV vectors. While, alternative Cas9 proteins have alternative targeting specificities, and are likely to be more restrictive than the SpCas9 protein, the use of the smaller saCas9 protein in combination with the H1 system offers significant advantages over the existing approach.

The CRISPR-Cas9 system functions by inducing a dsDNA break, however, a single point mutation in Cas9 can generate a ssDNA “nickase”. While use of a nickase requires twice as many gRNAs to generate a dsDNA break it is universally accepted as being safer. Importantly, the AAV-H1-CRISPR platform can accommodate SaCas9 and over four gRNAs, and can thus safely generate DNA breaks without off-target mutations. Current delivery approaches lack this capability.

Leber's congenital amaurosis (LCA) is comprised of a group of early-onset childhood retinal degenerations that are characterized by severe retinal dysfunction and severe visual impairment, or blindness during the first months of life. LCA, an orphan disease, is the most common cause of inherited blindness constituting as much as 5% of all known hereditary retinal degenerative diseases. Specifically, LCA10—a disease for which no FDA approved therapy exists—is caused by an intronic mutation in the CEP290 gene. This A-to-G mutation results in the creation of a de novo strong splice-donor site and the inclusion of a cryptic exon (Exon X) into the CEP290 mRNA and subsequent photoreceptor or ganglion degeneration. This mutation is a particularly attractive as a therapeutic target.

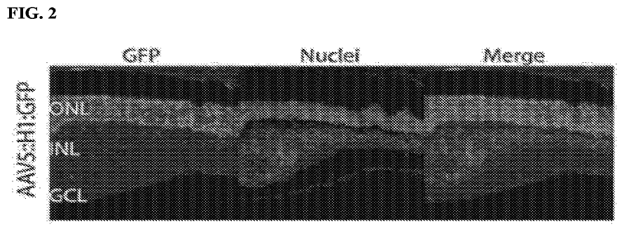

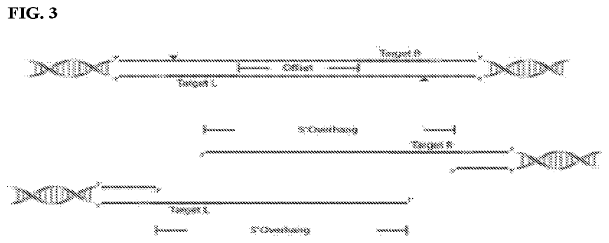

It was successfully demonstrated that prototypic AAV-H1-CRISPRs robustly express both Cas9 and a gRNA in human cells, and can thereby efficiently direct gene targeting in vitro. Using a mouse model of retinal disease, it was demonstrated that H1-AAV-CRISPR can be employed to precisely target pathogenic mutations in vivo, by subretinal delivery (FIG. 2).

An AAV-based strategy to treat the underlying cause of LCA10 with CRISPR is currently being developed for clinical use by Editas Medicine. Their approach to solving the AAV packaging capacity problem is to employ a smaller Cas9 ortholog from S. aureus (SaCas9), which is encoded by a ˜3.2 kb transcript. The compact size of the SaCas9 gene allows it to be packaged into a single AAV vector along with two gRNA cassettes, however our AAV technology provides additional room, which can be used for additional gRNAs.

In terms of safety concerns for CRISPR-based therapeutics, the most significant is undoubtedly off-target mutagenesis; this can occur if Cas9 cleaves DNA at an unintended location. Fortunately, this risk can be nearly eliminated by employing a modified form of Cas9 (known as the D10A nickase) that only cleaves one DNA strand. By separately engaging two gRNAs to generate two closely opposed nicks on opposite strands, the Cas9 nickase can efficiently generate a double strand break. However, an off-target break can only be generated by the nickase if the two gRNAs recognize off-targets that occur in close proximity and on opposite strands elsewhere in the genome, an occurrence that is statistically improbable (Church G. Nature 2015 Dec. 3; 528(7580):S7) and consequently not observed; nicked DNA is efficiently repaired but normal cellular proteins. For this reason, introducing a Cas9(D10A) with four gRNAs (a pair on each side to delete the targeted mutation) for correction of the CEP290 mutation would be viewed universally and unequivocally as the safest approach—including among regulatory agencies. (Shen et al. Nature Methods 11, 399-402 (2014)). The H1 element uniquely endows the space to use this capability.

However, due to the size limitations with the current system (i.e. Editas: saCas9 with the non-H1 system), correction of the CEP290 mutation, which involves dropping out approximately 1 kb of intronic DNA, cannot be done through the safer nickase approach. Four gRNA have been identified that would allow us to delivery a safer therapeutic to the clinic:

hs07571799[Sa]:

(SEQ ID NO: 1)

GATCTTATTCTACTCCTGTGAAAGGAT
hs07571796[Sa]:

(SEQ ID NO: 2)

AACGTTGTTCTGAGTAGCTTTCAGGAT
hs07571783[Sa]:

(SEQ ID NO: 3)

GATCATTCTTGTGGCAGTAAGGAGGAT
hs07571778[Sa]:

(SEQ ID NO: 4)

TCAAAAGCTACCGGTTACCTGAAGGGT

However, Editas has constrained their target sites to 5′ G targeting sites due to the U6 promoter. (Friedland et al. Genome Biology 16:257 (2015))

There is an spCas9 targeting site that overlaps with the A-to-G mutation. Using a previously disclosed H1 system and the spCas9, the CEP290 mutation can be directly targeted without need for generating a large deletion. This method would also be expected to be safer than the current Editas system.

WT
hs028893108;

(SEQ ID NO: 5)

GAGATATTCACAATTACAACTGG
LCA10
hs028893108*;

(SEQ ID NO: 6)

GAGATACTCACAATTACAACTGG

The reduced sites from the 5′G initiation requirement illustrates the utility in using the H1 to express the gRNAs over U6. Additionally, the total number of targeting sites shows that spCas9 outnumbers saCas9, as computationally predicted (Ranganathan et al. Nat Commun 2014 Aug. 8; 5:4516).

TABLE 2
saCas9 sites (1kb upstream and 1kb downstream CEP290 A-to-G mutation)

| chrom | Start | End | grid | strand | sequence |
|---|---|---|---|---|---|
| chr12 | 88494109 | 88494083 | hs07571772 [Sa] | − | AGAAATAGATGTAGATTGAGGTAGAAT (SEQ ID NO: 7) |
| chr12 | 88494180 | 88494154 | hs07571773 [Sa] | − | ATAAGGAAATACAAAAACTGGATGGGT (SEQ ID NO: 8) |
| chr12 | 88494184 | 88494158 | hs07571774 [Sa] | − | GATAATAAGGAAATACAAAAACTGGAT (SEQ TD NO: 9) |
| chr12 | 88494212 | 88494186 | hs07571775 [Sa] | − | TCAGATTTCATGTGTGAAGAATGGAAT (SEQ ID NO: 10) |
| chr12 | 88494217 | 88494191 | hs07571776 [Sa] | − | GAAAATCAGATTTCATGTGTGAAGAAT (SEQ ID NO: 11) |
| chr12 | 88494774 | 88494800 | hs07571777 [Sa] | + | CTTTTGACAGTTTTTAAGGCGGGGAGT (SEQ ID NO: 12) |
| chr12 | 88494780 | 88494754 | hs07571778 [Sa] | − | TCAAAAGCTACCGGTTACCTGAAGGGT (SEQ ID NO: 13) |
| chr12 | 88494785 | 88494811 | hs07571779 [Sal | + | TTTTAAGGCGGGGAGTCACATGGGAGT (SEQ ID NO: 14) |
| chr12 | 88494793 | 88494819 | hs07571780 [Sa] | + | CGGGGAGTCACATGGGAGTCACAGGGT (SEQ ID NO: 15) |
| chr12 | 88494798 | 88494824 | hs07571781 [Sa] | + | AGTCACATGGGAGTCACAGGGTAGGAT (SEQ ID NO: 16) |
| chr12 | 88494811 | 88494837 | hs07571782 [Sa] | + | TCACAGGGTAGGATTCATGTTTAGAAT (SEQ ID NO: 17) |
| chr12 | 88494838 | 88494864 | hs07571783 [Sa] | + | GATCATTCTTGTGGCAGTAAGGAGGAT (SEQ ID NO: 18) |
| chr12 | 88494849 | 88494823 | hs07571784 [Sa] | − | ACAAGAATGATCATTCTAAACATGAAT (SEQ ID NO: 19) |
| chr12 | 88494864 | 88494890 | hs07571785 [Sa] | + | TGTAAGACTGGAGATAGAGACAGGAAT (SEQ ID NO: 20) |
| chr12 | 88494868 | 88494842 | hs07571786 [Sa] | − | TTACATCCTCCTTACTGCCACAAGAAT (SEQ ID NO: 21) |
| chr12 | 88494921 | 88494947 | hs07571787 [Sa] | + | TAAAACTAAGACACTGCCAATAGGGAT (SEQ ID NO: 22) |
| chr12 | 88495501 | 88495475 | hs07571788 [Sa] | − | TTTAACGTTATCATTTTCCCAAAGAGT (SEQ ID NO: 23) |
| chr12 | 88495532 | 88495506 | hs07571789 [Sa] | − | CATCTTGTGGATAATGTATCAATGAGT (SEQ ID NO: 24) |
| chr12 | 88495541 | 88495567 | hs07571790 [Sa] | + | CTAGGACTTTCTAATGCTGGAGAGGAT (SEQ ID NO: 25) |
| chr12 | 88495547 | 88495521 | hs07571791 [Sa] | − | GTCCTAGGCAAGAGACATCTTGTGGAT (SEQ ID NO: 26) |
| chr12 | 88495562 | 88495588 | hs07571792 [Sa] | + | GAGGATAGGACAGAGGACATGGAGAAT (SEQ ID NO: 27) |
| chr12 | 88495714 | 88495740 | hs07571793 [Sa] | + | AAACATGACTCATAATTTAGTAGGAAT (SEQ ID NO: 28) |
| chr12 | 88495747 | 88495721 | hs07571794 [Sa] | − | TTTCAGGATTCCTACTAAATTATGAGT (SEQ ID NO: 29) |
| chr12 | 88495753 | 88495779 | hs07571795 [Sa] | + | TCAGAACAACGTTTTCATTTACTGAAT (SEQ ID NO: 30) |
| chr12 | 88495765 | 88495739 | hs07571796 [Sa] | − | AACGTTGTTCTGAGTAGCTTTCAGGAT (SEQ ID NO: 31) |
| chr12 | 88495777 | 88495751 | hs07571797 [Sa] | − | TCAGTAAATGAAAACGTTGTTCTGAGT (SEQ ID NO: 32) |
| chr12 | 88495794 | 88495820 | hs075/1/98 [Sa] | + | CAGCAAAAGCTTTTGAGCTAATAGAAT (SEQ ID NO: 33) |
| chr12 | 88495823 | 88495849 | hs07571799 [Sa] | + | GATCTTATTCTACTCCTGTGAAAGGAT (SEQ ID NO: 34) |
| chr12 | 88495836 | 88495862 | hs07571800 [Sa] | + | TCCTGTGAAAGGATCTTAGATAAGAAT (SEQ ID NO: 35) |
| chr12 | 88495855 | 88495829 | hs07571801 [Sa] | − | TCTAAGATCCTTTCACAGGAGTAGAAT (SEQ ID NO: 36) |
| chr12 | 88495860 | 88495834 | hs07571802 [Sa] | − | TCTTATCTAAGATCCTTTCACAGGAGT (SEQ ID NO: 37) |
| chr12 | 88495890 | 88495864 | hs07571803 [Sa] | − | AGAAATAGAGGCTTATGGATTTTGGAT (SEQ ID NO: 38) |
| chr12 | 88495897 | 88495871 | hs075/1804 [Sa] | − | CCTCATCAGAAATAGAGGCTTATGGAT (SEQ ID NO: 39) |
| chr12 | 88495921 | 88495947 | hs07571805 [Sa] | + | TTCTGTCCTCAGTAAAAGGTATAGAGT (SEQ ID NO: 40) |

TABLE 3
spCas9 sites (1kb upstream and 1kb downstream CEP290 A-to-G mutation)

| chrom | Start | End | grid | strand | sequence |
|---|---|---|---|---|---|
| chr12 | 88494095 | 88494073 | hs028893072 | − | ATTCAGCTAGAATCAACAAGAGG (SEQ ID NO: 41) |
| chr12 | 88494111 | 88494089 | hs028893073 | − | AAAGAAATAGATCTAGATTGACC (SEQ ID NO: 42) |
| chr12 | 88494177 | 88494155 | hs028893074 | − | AGGAAATACAAAAACTGGATGGG (SEQ ID NO: 43) |
| chr12 | 88494178 | 88494156 | hs028893075 | − | AAGGAAATACAAAAACTGGATGG (SEQ ID NO: 44) |
| chr12 | 88494182 | 88494160 | hs028893076 | − | TAATAAGGAAATACAAAAACTGG (SEQ ID NO: 45) |
| chr12 | 88494197 | 88494175 | hs028893077 | − | GAAGAATGGAATAGATAATAAGG (SEQ ID NO: 46) |
| chr12 | 88494211 | 88494189 | hs028893078 | − | CAGATTTCATGTGTGAAGAATGG (SEQ ID NO: 47) |
| chr12 | 88494244 | 88494222 | hs028893079 | − | GTAAAGGTTCATGAGACTAGAGG (SEQ ID NO: 48) |
| chr12 | 88494260 | 88494238 | hs028893080 | − | TGTTGCTTTTTGAGAGGTAAAGG (SEQ ID NO: 49) |
| chr12 | 88494266 | 88494244 | hs028893081 | − | AATTATTGTTGCTTTTTGAGAGG (SEQ ID NO: 50) |
| chr12 | 88494280 | 88494302 | hs028893082 | + | ATTTGACACCACATGCACTGTGG (SEQ ID NO: 51) |
| chr12 | 88494290 | 88494268 | hs028893083 | − | TGGTGTCAAATATGGTGCTTAGG (SEQ ID NO: 52) |
| chr12 | 88494298 | 88494276 | hs028893084 | − | AGTGCATGTGGTGTCAAATATGG (SEQ ID NO: 53) |
| chr12 | 88494310 | 88494288 | hs028893085 | − | GTGGAGAGCCACAGTGCATGTGG (SEQ ID NO: 54) |
| chr12 | 88494770 | 88494792 | hs028893086 | + | GTAGCTTTTGACAGTTTTTAAGG (SEQ ID NO: 55) |
| chr12 | 88494773 | 88494795 | hs028893087 | + | GCTTTTGACAGTTTTTAAGGCGG (SEQ ID NO: 56) |
| chr12 | 88494774 | 88494796 | hs028893088 | + | CTTTTGACAGTTTTTAAGGCGGG (SEQ ID NO: 57) |
| chr12 | 88494775 | 88494797 | hs028893089 | + | TTTTGACAGTTTTTAAGGCGGGG (SEQ ID NO: 58) |
| chr12 | 88494777 | 88494755 | hs028893090 | − | AAAGCTACCGGTTACCTGAAGGG (SEQ ID NO: 59) |
| chr12 | 88494778 | 88494756 | hs028893091 | − | AAAAGCTACCGGTTACCTGAAGG (SEQ ID NO: 60) |
| chr12 | 88494785 | 88494807 | hs028893092 | + | TTTTAAGGCGGGGAGTCACATGG (SEQ ID NO: 61) |
| chr12 | 88494786 | 88494808 | hs028893093 | + | TTTAAGGCGGGGAGTCACATGGG (SEQ ID NO: 62) |
| chr12 | 88494789 | 88494767 | hs028893094 | − | TAAAAACTGTCAAAAGCTACCGG (SEQ ID NO: 63) |
| chr12 | 88494795 | 88494817 | hs028893095 | + | GGGAGTCACATGGGAGTCACAGG (SEQ ID NO: 64) |
| chr12 | 88494796 | 88494818 | hs028893096 | + | GGAGTCACATGGGAGTCACAGGG (SEQ ID NO: 65) |
| chr12 | 88494800 | 88494822 | hs028893097 | + | TCACATGGGAGTCACAGGGTAGG (SEQ ID NO: 66) |
| chr12 | 88494829 | 88494851 | hs028893098 | + | GTTTAGAATGATCATTCTTGTGG (SFQ ID NO: 67) |
| chr12 | 88494837 | 88494859 | hs028893099 | + | TGATCATTCTTGTGGCAGTAAGG (SEQ ID NO: 68) |
| chr12 | 88494840 | 88494862 | hs028893100 | + | TCATTCTTGTGGCAGTAAGGAGG (SEQ ID NO: 69) |
| chr12 | 88494852 | 88494874 | hs028893101 | + | CAGTAAGGAGGATGTAAGACTGG (SEQ ID NO: 70) |
| chr12 | 88494865 | 88494887 | hs028893102 | + | GTAAGACTGGAGATAGAGACAGG (SEQ ID NO: 71) |
| chr12 | 88494873 | 88494895 | hs028893103 | + | GGAGATAGAGACAGGAATAATGG (SEQ ID NO: 72) |
| chr12 | 88494921 | 88494899 | hs028893104 | − | ATTTTTTATTATCTTTATTGTGG (SEQ ID NO: 73) |
| chr12 | 88494922 | 88494944 | hs028893105 | + | AAAACTAAGACACTGCCAATAGG (SEQ ID NO: 74) |
| chr12 | 88494923 | 88494945 | hs028893106 | + | AAACTAAGACACTGCCAATAGGG (SEQ ID NO: 75) |
| chr12 | 88494928 | 88494950 | hs028893107 | + | AAGACACTGCCAATAGGGATAGG (SEQ ID NO: 76) |
| chr12 | 88494954 | 88494976 | hs028893108 | + | GAGATATTCACAATTACAACTGG (SEQ ID NO: 77) |
| chr12 | 88494959 | 88494937 | hs028893109 | − | TATCTCATACCTATCCCTATTGG (SEQ ID NO: 78) |
| chr12 | 88495461 | 88495483 | hs028893110 | + | GAWAGATGAAAAATACTCTTTGG (SEQ ID NO: 79) |
| chr12 | 88495462 | 88495484 | hs028893111 | + | WWWGATGAAAAATACTCTTTGGG (SEQ ID NO: 80) |
| chr12 | 88495523 | 88495545 | hs028893112 | + | CCACAAGATGTCTCTTGCCTACC (SEQ ID NO: 81) |
| chr12 | 88495538 | 88495560 | hs028893113 | + | TCCCTAGCACTTTCTAATCCTCC (SEQ ID NO: 82) |
| chr12 | 88495543 | 88495565 | hs028893114 | + | AGGACTTTCTAATGCTGGAGAGG (SEQ ID NO: 83) |
| chr12 | 88495545 | 88495523 | hs028893115 | - | CCTAGCCAAGAGACATCTTCTCC (SEQ ID NO: 84) |
| chr12 | 88495548 | 88495570 | hs028893116 | + | TTTCTAATGCTGGAGAGGATAGG (SEQ ID NO: 85) |
| chr12 | 88495555 | 88495577 | hs028893117 | + | TGCTGGAGAGGATAGGACAGAGG (SEQ ID NO: 86) |
| chr12 | 88495561 | 88495583 | hs028893118 | + | AGAGGATAGGACAGAGGACATGG (SEQ ID NO: 87) |
| chr12 | 88495562 | 88495540 | hs028893119 | − | CTCCAGCATTAGAAAGTCCTAGG (SEQ ID NO: 88) |
| chr12 | 88495568 | 88495590 | hs028893120 | + | AGGACAGAGGACATGGAGAATGG (SEQ ID NO: 89) |
| chr12 | 88495599 | 88495621 | hs028893121 | + | AAATTATCACCACACTAAATAGG (SEQ ID NO: 90) |
| chr12 | 88495630 | 88495608 | hs028893122 | − | AAAATTATGCCTATTTAGTGTGG (SEQ ID NO: 91) |
| chr12 | 88495640 | 88495662 | hs028893123 | + | GTAACATAATCACCTCTCTTTGG (SEQ ID NO: 92) |
| chr12 | 88495674 | 88495652 | hs028893124 | − | CTGCTGCTTTTGCCAAAGAGAGG (SEQ ID NO: 93) |
| chr12 | 88495715 | 88495737 | hs028893125 | + | AACATGACTCATAATTTAGTAGG (SEQ ID NO: 94) |
| chr12 | 88495763 | 88495741 | hs028893126 | - | CGTTGTTCTGAGTAGCTTTCAGG (SEQ ID NO: 95) |
| chr12 | 88495815 | 88495793 | hs028893127 | − | ATTAGCTCAAAAGCTTTTGCTGG (SEQ ID NO: 96) |
| chr12 | 88495825 | 88495847 | hs028893128 | + | TCTTATTCTACTCCTGTGAAAGG (SEQ ID NO: 97) |
| chr12 | 88495859 | 88495837 | hs028893129 | − | CTTATCTAAGATCCTTTCACAGG (SEQ ID NO: 98) |
| chr12 | 88495875 | 88495897 | hs028893130 | + | ATAAGCCTCTATTTCTGATGAGG (SEQ ID NO: 99) |
| chr12 | 88495888 | 88495866 | hs028893131 | − | AAATAGAGGCTTATGGATTTTGG (SEQ ID NO: 100) |
| chr12 | 88495892 | 88495914 | hs028893132 | + | ATGAGGAAGATGAACAAATCAGG (SEQ ID NO: 101) |
| chr12 | 88495895 | 88495873 | hs028893133 | − | TCATCAGAAATAGAGGCTTATGG (SEQ ID NO: 102) |
| chr12 | 88495902 | 88495880 | hs028893134 | − | ATCTTCCTCATCAGAAATAGAGG (SEQ ID NO: 103) |
| chr12 | 88495917 | 88495939 | hs028893135 | + | CTTGTTCTGTCCTCAGTAAAAGG (SEQ ID NO: 104) |
| chr12 | 88495933 | 88495955 | hs028893136 | + | TAAAAGGTATAGAGTTCAAGTGG (SFQ ID NO: 105) |
| chr12 | 88495949 | 88495927 | hs028893137 | - | GAACTCTATACCTTTTACTGAGG (SFQ ID NO: 106) |
| chr12 | 88495953 | 88495975 | hs028893138 | + | TGGCTGTAAGATAACTACAAAGG (SEQ ID NO: 107) |
| chr12 | 88495954 | 88495976 | hs028893139 | + | GGCTGTAAGATAACTACAAAGGG (SEQ ID NO: 108) |
| chr12 | 88495959 | 88495981 | hs028893140 | + | TAAGATAACTACAAAGGGTCTGG (SEQ ID NO: 109) |

TABLE 4
CEP290 intronic targets

| | |
|---|---|
| ID | sequence |

SpCas9

| | |
|---|---|
| hs028892997 | ACAATGTCATTTTGTGGTATTGG (SEQ ID NO: 164) |
| hs028892998 | GTTAAGACAATGTCATTTTGTGG (SEQ ID NO: 165) |
| hs028892999 | AAACTGGATTGTGAGTTTTAAGG (SEQ ID NO: 166) |
| hs028893000 | ATTCTAATTTTGTAAAAAACTGG (SEQ ID NO: 167) |
| hs028893001 | ATCCTGTGTTATCCAATATACGG (SEQ ID NO: 168) |
| hs028893002 | ATATTGGATAACACAGGATTTGG (SEQ ID NO: 169) |
| hs028893003 | GGCCGTATATTGGATAACACAGG (SEQ ID NO: 170) |
| hs028893004 | CAGAAATTGAGGCCGTATATTGG (SEQ ID NO: 171) |
| hs028893005 | AAGCTAATACTCAGAAATTGAGG (SEQ ID NO: 172) |
| hs028893006 | CTAAAGCTGTACATTCATTTTGG (SEQ ID NO: 173) |
| hs028893007 | AAATGAATGTACAGCTTTAGTGG (SEQ ID NO: 174) |
| hs028893008 | CTTGAGTATTCTAAGAGTTTTGG (SEQ ID NO: 175) |
| hs028893009 | AAGTAAGCACTAGTTTGTGAAGG (SEQ ID NO: 176) |
| hs028893010 | CTCAGCAACTGTGTGCATGAAGG (SEQ ID NO: 177) |
| hs028893011 | TCTGTCACTGATGGCACACTAGG (SEQ ID NO: 178) |
| hs028893012 | ACATCATCATCTGTCACTGATGG (SEQ ID NO: 179) |
| hs028893013 | TGAAAAGAAATATAAAATTCTGG (SEQ ID NO: 180) |
| hs028893017 | CTGAATGGGCAGAACTCTGAAGG (SEQ ID NO: 181) |
| hs028893014 | ATTCAGTGCCTATGTGTGTGTGG (SEQ ID NO: 182) |
| hs028893015 | TTCAGTGCCTATGTGTGTGTGGG (SEQ ID NO: 183) |
| hs028893016 | AGTGCCTATGTGTGTGTGGGTGG (SEQ ID NO: 184) |
| hs028893018 | GTGCCTATGTGTGTGTGGGTGGG (SEQ ID NO: 185) |
| hs028893019 | CCTATGTGTGTGTGGGTGGGTGG (SEQ ID NO: 186) |
| hs028893021 | CACACACATAGGCACTGAATGGG (SEQ ID NO: 187) |
| hs028893022 | ACACACACATAGGCACTGAATGG (SEQ ID NO: 188) |
| hs028893020 | TGTGTGTGTGGGTGGGTGGCAGG (SEQ ID NO: 189) |
| hs028893024 | CCACCCACCCACACACACATAGG (SEQ ID NO: 190) |
| hs028893023 | GGCAGGATTTCGCCAGAGTGTGG (SEQ ID NO: 191) |
| hs028893025 | GGATTTCGCCAGAGTGTGGAAGG (SEQ ID NO: 192) |
| hs028893026 | ATCTGCTGCCTTCCACACTCTGG (SEQ ID NO: 193) |
| hs028893027 | TGAGCCAGTGAATAATAGAAAGG (SEQ ID NO: 194) |
| hs028893030 | TGTTCCTTTCTATTATTCACTGG (SEQ ID NO: 195) |
| hs028893028 | AAAGGAACATTTAGATAGAGCGG (SEQ ID NO: 196) |
| hs028893029 | CATTTAGATAGAGCGGTTGAAGG (SEQ ID NO: 197) |
| hs028893032 | CTTAAATATCAATTATTTGGTGG (SEQ ID NO: 198) |
| hs028893031 | GATATTTAAGCTGAACTGTGAGG (SEQ ID NO: 199) |
| hs028893033 | CAGCTTAAATATCAATTATTTGG (SEQ ID NO: 200) |
| hs028893034 | AGTAAACCCAGATGCTCCAAAGG (SEQ ID NO: 201) |
| hs028893035 | CTCCAAAGGAAGAAAGCATTTGG (SEQ ID NO: 202) |
| hs028893036 | TTTCTTCCTTTGGAGCATCTGGG (SEQ ID NO: 203) |
| hs028893037 | CTTTCTTCCTTTGGAGCATCTGG (SEQ ID NO: 204) |
| hs028893039 | TTCCAAATGCTTTCTTCCTTTGG (SEQ ID NO: 205) |
| hs028893038 | GGAATACTATAAGATCTGAGAGG (SEQ ID NO: 206) |
| hs028893040 | GAAAAAATGATTGAAATGATTGG (SEQ ID NO: 207) |
| hs028893041 | ATGATTGAAATGATTGGAGCAGG (SEQ ID NO: 208) |
| hs028893042 | GATTGGAGCAGGAAATGATTTGG (SEQ ID NO: 209) |
| hs028893043 | TGGAGCAGGAAATGATTTGGAGG (SEQ ID NO: 210) |
| hs028893044 | CAGGAAATGATTTGGAGGACTGG (SEQ ID NO: 211) |
| hs028893045 | TTTATATACTATGAATACACAGG (SEQ ID NO: 212) |
| hs028893046 | ATATACTATGAATACACAGGAGG (SEQ ID NO: 213) |
| hs028893047 | TATACTATGAATACACAGGAGGG (SEQ ID NO: 214) |
| hs028893048 | GAATACACAGGAGGGAAAACAGG (SEQ ID NO: 215) |
| hs028893049 | TACACAGGAGGGAAAACAGGAGG (SEQ ID NO: 216) |
| hs028893050 | GGAGGGAAAACAGGAGGCTGAGG (SEQ ID NO: 217) |
| hs028893051 | GAGGGAAAACAGGAGGCTGAGGG (SEQ ID NO: 218) |
| hs028893052 | TTCCTTTCATCCTTTTTCACAGG (SEQ ID NO: 219) |
| hs028893053 | GGATGAAAGGAAGAAAAAAATGG (SEQ ID NO: 220) |
| hs028893054 | ATCCTGTGAAAAAGGATGAAAGG (SEQ ID NO: 221) |
| hs028893055 | ATAGGATTATCCTGTGAAAAAGG (SEQ ID NO: 222) |
| hs028893056 | AGAGATTTGTAACAAACAATAGG (SEQ ID NO: 223) |
| hs028893057 | AGCAAAATATCAGGGTAGGTGGG (SEQ ID NO: 224) |
| hs028893058 | TAGCAAAATATCAGGGTAGGTGG (SEQ ID NO: 225) |
| hs028893059 | AAATAGCAAAATATCAGGGTAGG (SEQ ID NO: 226) |
| hs028893060 | AGGGAAATAGCAAAATATCAGGG (SEQ ID NO: 227) |
| hs028893061 | AAGGGAAATAGCAAAATATCAGG (SEQ ID NO: 228) |
| hs028893062 | AGAGATGAAAGTGGTAATAAGGG (SEQ ID NO: 229) |
| hs028893063 | AAGAGATGAAAGTGGTAATAAGG (SEQ ID NO: 230) |
| hs028893064 | ATGAAGATAAGAGATGAAAGTGG (SEQ ID NO: 231) |
| hs028893065 | AAGAAAATTTAGAGTTACTAGGG (SEQ ID NO: 232) |
| hs028893066 | AAAGAAAATTTAGAGTTACTAGG (SEQ ID NO: 233) |
| hs028893067 | CCCTCCTCCTGAAACTTCACTGG (SEQ ID NO: 234) |
| hs028893068 | CCAGTGAAGTTTCAGGAGGAGGG (SEQ ID NO: 235) |
| hs028893069 | ACCAGTGAAGTTTCAGGAGGAGG (SEQ ID NO: 236) |
| hs028893070 | GAGACCAGTGAAGTTTCAGGAGG (SEQ ID NO: 237) |
| hs028893071 | AAGGAGACCAGTGAAGTTTCAGG (SEQ ID NO: 238) |
| hs028893072 | ATTGAGGTAGAATCAAGAAGAGG (SEQ ID NO: 239) |
| hs028893073 | AAAGAAATAGATGTAGATTGAGG (SEQ ID NO: 240) |
| hs028893074 | AGGAAATACAAAAACTGGATGGG (SEQ ID NO: 241) |
| hs028893075 | AAGGAAATACAAAAACTGGATGG (SEQ ID NO: 242) |
| hs028893076 | TAATAAGGAAATACAAAAACTGG (SEQ ID NO: 243) |
| hs028893077 | GAAGAATGGAATAGATAATAAGG (SEQ ID NO: 244) |
| hs028893078 | CAGATTTCATGTGTGAAGAATGG (SEQ ID NO: 245) |
| hs028893079 | GTAAAGGTTCATGAGACTAGAGG (SEQ ID NO: 246) |
| hs028893080 | TGTTGCTTTTTGAGAGGTAAAGG (SEQ ID NO: 247) |
| hs028893081 | AATTATTGTTGCTTTTTGAGAGG (SEQ ID NO: 248) |
| hs028893082 | ATTTGACACCACATGCACTGTGG (SEQ ID NO: 249) |
| hs028893083 | TGGTGTCAAATATGGTGCTTAGG (SEQ ID NO: 250) |
| hs028893084 | AGTGCATGTGGTGTCAAATATGG (SEQ ID NO: 251) |
| hs028893085 | GTGGAGAGCCACAGTGCATGTGG (SEQ ID NO: 252) |
| hs028893090 | AAAGCTACCGGTTACCTGAAGGG (SEQ ID NO: 253) |
| hs028893091 | AAAAGCTACCGGTTACCTGAAGG (SEQ ID NO: 254) |
| hs028893086 | GTAGCTTTTGACAGTTTTTAAGG (SEQ ID NO: 255) |
| hs028893087 | GCTTTTGACAGTTTTTAAGGCGG (SEQ ID NO: 256) |
| hs028893088 | CTTTTGACAGTTTTTAAGGCGGG (SEQ ID NO: 257) |
| hs028893089 | TTTTGACAGTTTTTAAGGCGGGG (SEQ ID NO: 258) |
| hs028893094 | TAAAAACTGTCAAAAGCTACCGG (SEQ ID NO: 259) |
| hs028893092 | TTTTAAGGCGGGGAGTCACATGG (SEQ ID NO: 260) |
| hs028893093 | TTTAAGGCGGGGAGTCACATGGG (SEQ ID NO: 261) |
| hs028893095 | GGGAGTCACATGGGAGTCACAGG (SEQ ID NO: 262) |
| hs028893096 | GGAGTCACATGGGAGTCACAGGG (SEQ ID NO: 263) |
| hs028893097 | TCACATGGGAGTCACAGGGTAGG (SEQ ID NO: 264) |
| hs028893098 | GTTTAGAATGATCATTCTTGTGG (SEQ ID NO: 265) |
| hs028893099 | TGATCATTCTTGTGGCAGTAAGG (SEQ ID NO: 266) |
| hs028893100 | TCATTCTTGTGGCAGTAAGGAGG (SEQ ID NO: 267) |
| hs028893101 | CAGTAAGGAGGATGTAAGACTGG (SEQ ID NO: 268) |
| hs028893102 | GTAAGACTGGAGATAGAGACAGG (SEQ ID NO: 269) |
| hs028893103 | GGAGATAGAGACAGGAATAATGG (SEQ ID NO: 270) |
| hs028893104 | ATTTTTTATTATCTTTATTGTGG (SEQ ID NO: 271) |
| hs028893105 | AAAACTAAGACACTGCCAATAGG (SEQ ID NO: 272) |
| hs028893106 | AAACTAAGACACTGCCAATAGGG (SEQ ID NO: 273) |
| hs028893107 | AAGACACTGCCAATAGGGATAGG (SEQ ID NO: 274) |
| hs028893109 | TATCTCATACCTATCCCTATTGG (SEQ ID NO: 275) |
| hs028893108 | GAGATATTCACAATTACAACTGG (SEQ ID NO: 276) |
| hs028893110 | GAAAGATGAAAAATACTCTTTGG (SEQ ID NO: 277) |
| hs028893111 | AAAGATGAAAAATACTCTTTGGG (SEQ ID NO: 278) |
| hs028893112 | CCACAAGATGTCTCTTGCCTAGG (SEQ ID NO: 279) |
| hs028893115 | CCTAGGCAAGAGACATCTTGTGG (SEQ ID NO: 280) |
| hs028893113 | TGCCTAGGACTTTCTAATGCTGG (SEQ ID NO: 281) |
| hs028893114 | AGGACTTTCTAATGCTGGAGAGG (SEQ ID NO: 282) |
| hs028893116 | TTTCTAATGCTGGAGAGGATAGG (SEQ ID NO: 283) |
| hs028893119 | CTCCAGCATTAGAAAGTCCTAGG (SEQ ID NO: 284) |
| hs028893117 | TGCTGGAGAGGATAGGACAGAGG (SEQ ID NO: 285) |
| hs028893118 | AGAGGATAGGACAGAGGACATGG (SEQ ID NO: 286) |
| hs028893120 | AGGACAGAGGACATGGAGAATGG (SEQ ID NO: 287) |
| hs028893121 | AAATTATCACCACACTAAATAGG (SEQ ID NO: 288) |
| hs028893122 | AAAATTATGCCTATTTAGTGTGG (SEQ ID NO: 289) |
| hs028893123 | GTAACATAATCACCTCTCTTTGG (SEQ ID NO: 290) |
| hs028893124 | CTGCTGCTTTTGCCAAAGAGAGG (SEQ ID NO: 291) |
| hs028893125 | AACATGACTCATAATTTAGTAGG (SEQ ID NO: 292) |
| hs028893126 | CGTTGTTCTGAGTAGCTTTCAGG (SEQ ID NO: 293) |
| hs028893127 | ATTAGCTCAAAAGCTTTTGCTGG (SEQ ID NO: 294) |
| hs028893128 | TCTTATTCTACTCCTGTGAAAGG (SEQ ID NO: 295) |
| hs028893129 | CTTATCTAAGATCCTTTCACAGG (SEQ ID NO: 296) |
| hs028893130 | ATAAGCCTCTATTTCTGATGAGG (SEQ ID NO: 297) |
| hs028893131 | AAATAGAGGCTTATGGATTTTGG (SEQ ID NO: 298) |
| hs028893133 | TCATCAGAAATAGAGGCTTATGG (SEQ ID NO: 299) |
| hs028893132 | ATGAGGAAGATGAACAAATCAGG (SEQ ID NO: 300) |
| hs028893134 | ATCTTCCTCATCAGAAATAGAGG (SEQ ID NO: 301) |
| hs028893135 | CTTGTTCTGTCCTCAGTAAAAGG (SEQ ID NO: 302) |
| hs028893136 | TAAAAGGTATAGAGTTCAAGTGG (SEQ ID NO: 303) |
| hs028893137 | GAACTCTATACCTTTTACTGAGG (SEQ ID NO: 304) |
| hs028893138 | TGGCTGTAAGATAACTACAAAGG (SEQ ID NO: 305) |
| hs028893139 | GGCTGTAAGATAACTACAAAGGG (SEQ ID NO: 306) |
| hs028893140 | TAAGATAACTACAAAGGGTCTGG (SEQ ID NO: 307) |
| hs028893141 | TAAGTTACAATCTGTGAATATGG (SEQ ID NO: 308) |
| hs028893142 | GAATTTACAGAGTGCATCCATGG (SEQ ID NO: 309) |
| hs028893143 | ACAGAGTGCATCCATGGTCCAGG (SEQ ID NO: 310) |
| hs028893145 | TAAGGGCTCTTCCTGGACCATGG (SEQ ID NO: 311) |
| hs028893144 | CTTAATATTTCAGCTACTGTTGG (SEQ ID NO: 312) |
| hs028893146 | GAAATATTAAGGGCTCTTCCTGG (SEQ ID NO: 313) |
| hs028893148 | AACAGTAGCTGAAATATTAAGGG (SEQ ID NO: 314) |
| hs028893149 | CAACAGTAGCTGAAATATTAAGG (SEQ ID NO: 315) |
| hs028893147 | TGTTGGCTACATCCATTCCAAGG (SEQ ID NO: 316) |
| hs028893150 | CATTCCAAGGAACAAAAGCCAGG (SEQ ID NO: 317) |
| hs028893151 | ATTCCAAGGAACAAAAGCCAGGG (SEQ ID NO: 318) |
| hs028893152 | GGAACAAAAGCCAGGGACCATGG (SEQ ID NO: 319) |
| hs028893153 | GAACAAAAGCCAGGGACCATGGG (SEQ ID NO: 320) |
| hs028893154 | CTGGCTTTTGTTCCTTGGAATGG (SEQ ID NO: 321) |
| hs028893155 | GGTCCCTGGCTTTTGTTCCTTGG (SEQ ID NO: 322) |
| hs028893156 | CATGGGAGAATAGTTTGTTCTGG (SEQ ID NO: 323) |
| hs028893157 | ATGGGAGAATAGTTTGTTCTGGG (SEQ ID NO: 324) |
| hs028893161 | AACTATTCTCCCATGGTCCCTGG (SEQ ID NO: 325) |
| hs028893158 | GAATAGTTTGTTCTGGGTACAGG (SEQ ID NO: 326) |
| hs028893159 | AATAGTTTGTTCTGGGTACAGGG (SEQ ID NO: 327) |
| hs028893160 | ATAGTTTGTTCTGGGTACAGGGG (SEQ ID NO: 328) |
| hs028893163 | CAGAACAAACTATTCTCCCATGG (SEQ ID NO: 329) |
| hs028893162 | TGGGTACAGGGGTAAGAGAAAGG (SEQ ID NO: 330) |
| hs028893164 | GGGTACAGGGGTAAGAGAAAGGG (SEQ ID NO: 331) |
| hs028893165 | ACAGGGGTAAGAGAAAGGGATGG (SEQ ID NO: 332) |
| hs028893166 | CAGGGGTAAGAGAAAGGGATGGG (SEQ ID NO: 333) |
| hs028893167 | AGAATATTGTAATCAAAGGAGGG (SEQ ID NO: 334) |
| hs028893168 | TAGAATATTGTAATCAAAGGAGG (SEQ ID NO: 335) |
| hs028893169 | AGGTAGAATATTGTAATCAAAGG (SEQ ID NO: 336) |
| hs028893170 | TCCTTTTTCTCTTAGATGTCTGG (SEQ ID NO: 337) |
| hs028893171 | GAGAAAAAGGAGCATGAAACAGG (SEQ ID NO: 38) |
| hs028893172 | CTTAGATGTCTGGTTAAAAGAGG (SEQ ID NO: 339) |
| hs028893173 | ACCAGACATCTAAGAGAAAAAGG (SEQ ID NO: 340) |
| hs028893174 | TAAATCATGCAAGTGACCTAAGG (SEQ ID NO: 341) |
| hs028893175 | TTATCTAGATCATTCTCCTTAGG (SEQ ID NO: 342) |
| hs028893176 | AATATATTATCTATTTATAGTGG (SEQ ID NO: 343) |
| hs028893177 | GTGTGTGTGTGTGTGTTATGTGG (SEQ ID NO: 344) |
| hs028893178 | TGTGTGTGTGTGTGTTATGTGGG (SEQ ID NO: 345) |
| hs028893179 | ACTAAACTGAATGAATAATATGG (SEQ ID NO: 346) |
| hs028893180 | TGTTAAATTTATATAAATGCAGG (SEQ ID NO: 347) |
| hs028893182 | TTTTTGTGTTTGGGGGGATTTGG (SEQ ID NO: 348) |
| hs028893181 | ACAAAAATATTGAAGAAAAATGG (SEQ ID NO: 349) |
| hs028893183 | TCAATATTTTTGTGTTTGGGGGG (SEQ ID NO: 350) |
| hs028893184 | TTCAATATTTTTGTGTTTGGGGG (SEQ ID NO: 351) |
| hs028893185 | CTTCAATATTTTTGTGTTTGGGG (SEQ ID NO: 352) |
| hs028893187 | TCTTCAATATTTTTGTGTTTGGG (SEQ ID NO: 353) |
| hs028893188 | TTCTTCAATATTTTTGTGTTTGG (SEQ ID NO: 354) |
| hs028893186 | GAAAAATGGCTAATTTCACAAGG (SEQ ID NO: 355) |
| hs028893189 | AATCACACAAACTTACCTCCAGG (SEQ ID NO: 356) |

SaCas9

| | |
|---|---|
| hs07571737[Sa] | ATGTCATTTTGTGGTATTGGTCTGAAT (SEQ ID NO: 357) |
| hs07571738[Sa] | CAATCCAGTTTTTTACAAAATTAGAAT (SEQ ID NO: 358) |
| hs07571739[Sa] | AATTTTGTAAAAAACTGGATTGTGAGT (SEQ ID NO: 359) |
| hs07571740[Sa] | AAATTCTAATTTTGTAAAAAACTGGAT (SEQ ID NO: 360) |
| hs07571741[Sa] | TCCAATATACGGCCTCAATTTCTGAGT (SEQ ID NO: 361) |
| hs07571742[Sa] | GTATATTGGATAACACAGGATTTGGAT (SEQ ID NO: 362) |
| hs07571743[Sa] | GAGGCCGTATATTGGATAACACAGGAT (SEQ ID NO: 363) |
| hs07571744[Sa] | CTCAGAAATTGAGGCCGTATATTGGAT (SEQ ID NO: 364) |
| hs07571745[Sa] | ATTCATTTTGGCCAAAACTCTTAGAAT (SEQ ID NO: 365) |
| hs07571746[Sa] | GCCAAAACTCTTAGAATACTCAAGAGT (SEQ ID NO: 366) |
| hs07571747[Sa] | ATTCTAAGAGTTTTGGCCAAAATGAAT (SEQ ID NO: 367) |
| hs07571748[Sa] | TGCAAAACTCTTGAGTATTCTAAGAGT (SEQ ID NO: 368) |
| hs07571749[Sa] | TTTGTGAAGGCTGCAAAACTCTTGAGT (SEQ ID NO: 369) |
| hs07571750[Sa] | CATCTGTCACTGATGGCACACTAGGAT (SEQ ID NO: 370) |
| hs07571751[Sa] | GAAATATAAAATTCTGGCCTTCAGAGT (SEQ ID NO: 371) |
| hs07571752[Sa] | CCATTCAGTGCCTATGTGTGTGTGGGT (SEQ ID NO: 372) |
| hs07571753[Sa] | AATGGGCAGAACTCTGAAGGCCAGAAT (SEQ ID NO: 373) |
| hs07571754[Sa] | TCAGTGCCTATGTGTGTGTGGGTGGGT (SEQ ID NO: 374) |
| hs07571755[Sa] | TATGTGTGTGTGGGTGGGTGGCAGGAT (SEQ ID NO: 375) |
| hs07571756[Sa] | GGGTGGGTGGCAGGATTTCGCCAGAGT (SEQ ID NO: 376) |
| hs07571757[Sa] | CCACCCACACACACATAGGCACTGAAT (SEQ ID NO: 377) |
| hs07571758[Sa] | CATTTAGAAAGAAAATAAAAACAGAGT (SEQ ID NO: 378) |
| hs07571759[Sa] | TAAAAACAGAGTACTGAGCCAGTGAAT (SEQ ID NO: 379) |
| hs07571760[Sa] | AACATTTAGATAGAGCGGTTGAAGGAT (SEQ ID NO: 380) |
| hs07571761[Sa] | GCTCCAAAGGAAGAAAGCATTTGGAAT (SEQ ID NO: 381) |
| hs07571762[Sa] | TGCTTTCTTCCTTTGGAGCATCTGGGT (SEQ ID NO: 382) |
| hs07571763[Sa] | ATAATTATAGTTTTATATACTATGAAT (SEQ ID NO: 382) |
| hs07571764[Sa] | CAGGAGGGAAAACAGGAGGCTGAGGGT (SEQ ID NO: 384) |
| hs07571765[Sa] | TCTTCCTTTCATCCTTTTTCACAGGAT (SEQ ID NO: 385) |
| hs07571766[Sa] | AAGGATGAAAGGAAGAAAAAAATGGAT (SEQ ID NO: 386) |
| hs07571767[Sa] | CAATAGGATTATCCTGTGAAAAAGGAT (SEQ ID NO: 387) |
| hs07571768[Sa] | AAAGAGATTTGTAACAAACAATAGGAT (SEQ ID NO: 388) |
| hs07571769[Sa] | ATAAGGGAAATAGCAAAATATCAGGGT (SEQ ID NO: 389) |
| hs07571770[Sa] | AAAATTTAGAGTTACTAGGGAGAGAGT (SEQ ID NO: 390) |
| hs07571771[Sa] | CAGGAGGAGGGAAAGAAAATTTAGAGT (SEQ ID NO: 391) |
| hs07571772[Sa] | AGAAATAGATGTAGATTGAGGTAGAAT (SEQ ID NO: 392) |
| hs07571773[Sa] | ATAAGGAAATACAAAAACTGGATGGGT (SEQ ID NO: 393) |
| hs07571774[Sa] | GATAATAAGGAAATACAAAAACTGGAT (SEQ ID NO: 394) |
| hs07571775[Sa] | TCAGATTTCATGTGTGAAGAATGGAAT (SEQ ID NO: 395) |
| hs07571776[Sa] | GAAAATCAGATTTCATGTGTGAAGAAT (SEQ ID NO: 396) |
| hs07571777[Sa] | CTTTTGACAGTTTTTAAGGCGGGGAGT (SEQ ID NO: 397) |
| hs07571778[Sa] | TCAAAAGCTACCGGTTACCTGAAGGGT (SEQ ID NO: 398) |
| hs07571779[Sa] | TTTTAAGGCGGGGAGTCACATGGGAGT (SEQ ID NO: 399) |
| hs07571780[Sa] | CGGGGAGTCACATGGGAGTCACAGGGT (SEQ ID NO: 400) |
| hs07571781[Sa] | AGTCACATGGGAGTCACAGGGTAGGAT (SEQ ID NO: 401) |
| hs07571782[Sa] | TCACAGGGTAGGATTCATGTTTAGAAT (SEQ ID NO: 402) |
| hs07571783[Sa] | GATCATTCTTGTGGCAGTAAGGAGGAT (SEQ ID NO: 403) |
| hs07571784[Sa] | ACAAGAATGATCATTCTAAACATGAAT (SEQ ID NO: 404) |
| hs07571785[Sa] | TGTAAGACTGGAGATAGAGACAGGAAT (SEQ ID NO: 405) |
| hs07571786[Sa] | TTACATCCTCCTTACTGCCACAAGAAT (SEQ ID NO: 406) |
| hs07571787[Sa] | TAAAACTAAGACACTGCCAATAGGGAT (SEQ ID NO: 407) |
| hs07571788[Sa] | TTTAACGTTATCATTTTCCCAAAGAGT (SEQ ID NO: 408) |
| hs07571789[Sa] | CATCTTGTGGATAATGTATCAATGAGT (SEQ ID NO: 409) |
| hs07571790[Sa] | CTAGGACTTTCTAATGCTGGAGAGGAT (SEQ ID NO: 410) |
| hs07571791[Sa] | GTCCTAGGCAAGAGACATCTTGTGGAT (SEQ ID NO: 411) |
| hs07571792[Sa] | GAGGATAGGACAGAGGACATGGAGAAT (SEQ ID NO: 412) |
| hs07571793[Sa] | AAACATGACTCATAATTTAGTAGGAAT (SEQ ID NO: 413) |
| hs07571794[Sa] | TTTCAGGATTCCTACTAAATTATGAGT (SEQ ID NO: 414) |
| hs07571795[Sa] | TCAGAACAACGTTTTCATTTACTGAAT (SEQ ID NO: 415) |
| hs07571796[Sa] | AACGTTGTTCTGAGTAGCTTTCAGGAT (SEQ ID NO: 416) |
| hs07571797[Sa] | TCAGTAAATGAAAACGTTGTTCTGAGT (SEQ ID NO: 417) |
| hs07571798[Sa] | CAGCAAAAGCTTTTGAGCTAATAGAAT (SEQ ID NO: 418) |
| hs07571799[Sa] | GATCTTATTCTACTCCTGTGAAAGGAT (SEQ ID NO: 419) |
| hs07571800[Sa] | TCCTGTGAAAGGATCTTAGATAAGAAT (SEQ ID NO: 420) |
| hs07571801[Sa] | TCTAAGATCCTTTCACAGGAGTAGAAT (SEQ ID NO: 421) |
| hs07571802[Sa] | TCTTATCTAAGATCCTTTCACAGGAGT (SEQ ID NO: 422) |
| hs07571803[Sa] | AGAAATAGAGGCTTATGGATTTTGGAT (SEQ ID NO: 423) |
| hs07571804[Sa] | CCTCATCAGAAATAGAGGCTTATGGAT (SEQ ID NO: 424) |
| hs07571805[Sa] | TTCTGTCCTCAGTAAAAGGTATAGAGT (SEQ ID NO: 425) |
| hs07571806[Sa] | AGTGGCTGTAAGATAACTACAAAGGGT (SEQ ID NO: 426) |
| hs07571807[Sa] | GAATATCATAAGTTACAATCTGTGAAT (SEQ ID NO: 427) |
| hs07571808[Sa] | TATTCTACAATAAAAAATGATGAGAAT (SEQ ID NO: 428) |
| hs07571809[Sa] | ATAAAAAATGATGAGAATTTACAGAGT (SEQ ID NO: 429) |
| hs07571810[Sa] | TTTTTTATTGTAGAATAAATGTAGAAT (SEQ ID NO: 430) |
| hs07571811[Sa] | AATTCTCATCATTTTTTATTGTAGAAT (SEQ ID NO: 431) |
| hs07571812[Sa] | ATTAAGGGCTCTTCCTGGACCATGGAT (SEQ ID NO: 432) |
| hs07571813[Sa] | AACAAAAGCCAGGGACCATGGGAGAAT (SEQ ID NO: 433) |
| hs07571814[Sa] | CCCTGGCTTTTGTTCCTTGGAATGGAT (SEQ ID NO: 434) |
| hs07571815[Sa] | ACCATGGGAGAATAGTTTGTTCTGGGT (SEQ ID NO: 435) |
| hs07571816[Sa] | TGGTCCCTGGCTTTTGTTCCTTGGAAT (SEQ ID NO: 436) |
| hs07571817[Sa] | AGAATAGTTTGTTCTGGGTACAGGGGT (SEQ ID NO: 437) |
| hs07571818[Sa] | CTGGGTACAGGGGTAAGAGAAAGGGAT (SEQ ID NO: 438) |
| hs07571819[Sa] | AGAGAAAGGGATGGGCACTTAATGAGT (SEQ ID NO: 439) |
| hs07571820[Sa] | GAAAAAGGAGCATGAAACAGGTAGAAT (SEQ ID NO: 440) |
| hs07571821[Sa] | AAATCATGCAAGTGACCTAAGGAGAAT (SEQ ID NO: 441) |
| hs07571822[Sa] | TAGGTCACTTGCATGATTTAGCTGAAT (SEQ ID NO: 442) |
| hs07571823[Sa] | TTTATAGTGGCTGAATGACTTCTGAAT (SEQ ID NO: 443) |
| hs07571824[Sa] | TATATTATCTATTTATAGTGGCTGAAT (SEQ ID NO: 444) |
| hs07571825[Sa] | AACAAAATATTTATAACTAAACTGAAT (SEQ ID NO: 445) |
| hs07571826[Sa] | AAATATTTATAACTAAACTGAATGAAT (SEQ ID NO: 446) |
| hs07571827[Sa] | TAACTAAACTGAATGAATAATATGGAT (SEQ ID NO: 447) |
| hs07571828[Sa] | CTTCAATATTTTTGTGTTTGGGGGGAT (SEQ ID NO: 448) |
| hs07571829[Sa] | ATGGCTAATTTCACAAGGTTCAAGAAT (SEQ ID NO: 449) |

StCas9

| | |
|---|---|
| hs02772544[St] | ATCCAGTTTTTTACAAAATTAGAAT (SEQ ID NO: 450) |
| hs02772545[St] | AGTGGCAGAAGCTAATACTCAGAAA (SEQ ID NO: 451) |
| hs02772546[St] | TCATTTTGGCCAAAACTCTTAGAAT (SEQ ID NO: 452) |
| hs02772547[St] | TGATGATGTTTACAGTGAAAAGAAA (SEQ ID NO: 453) |
| hs02772548[St] | TGGGCAGAACTCTGAAGGCCAGAAT (SEQ ID NO: 454) |
| hs02772549[St] | GTACTGAGCCAGTGAATAATAGAAA (SEQ ID NO: 455) |
| hs02772550[St] | AACCCAGATGCTCCAAAGGAAGAAA (SEQ ID NO: 456) |
| hs02772551[St] | AAAGGAAGAAAAAAATGGATAGAAA (SEQ ID NO: 457) |
| hs02772552[St] | TGTGAAAAAGGATGAAAGGAAGAAA (SEQ ID NO: 458) |
| hs02772553[St] | AAAATATCAGGGTAGGTGGGAGAAA (SEQ ID NO: 459) |
| hs02772554[St] | GAAGTTTCAGGAGGAGGGAAAGAAA (SEQ ID NO: 460) |
| hs02772555[St] | AAATAGATGTAGATTGAGGTAGAAT (SEQ ID NO: 461) |
| hs02772556[St] | ATTTGATAGTAGAAGAAAAAAGAAA (SEQ ID NO: 462) |
| hs02772557[St] | ACTTGAAATTTGATAGTAGAAGAAA (SEQ ID NO: 463) |
| hs02772558[St] | TGGATGGGTAATAAAGCAAAAGAAA (SEQ ID NO: 464) |
| hs02772559[St] | AAATCAGATTTCATGTGTGAAGAAT (SEQ ID NO: 465) |
| hs02772560[St] | ACAGGGTAGGATTCATGTTTAGAAT (SEQ ID NO: 466) |
| hs02772561[St] | ACATCCTCCTTACTGCCACAAGAAT (SEQ ID NO: 467) |
| hs02772562[St] | GGATAGGACAGAGGACATGGAGAAT (SEQ ID NO: 468) |
| hs02772563[St] | GTCCTATCCTCTCCAGCATTAGAAA (SEQ ID NO: 469) |
| hs02772564[St] | GTGATTATGTTACTTTTTATAGAAA (SEQ ID NO: 470) |
| hs02772565[St] | CTCTCTTTGGCAAAAGCAGCAGAAA (SEQ ID NO: 471) |
| hs02772566[St] | GCAAAAGCTTTTGAGCTAATAGAAT (SEQ ID NO: 472) |
| hs02772567[St] | CTGTGAAAGGATCTTAGATAAGAAT (SEQ ID NO: 473) |
| hs02772568[St] | TAAGATCCTTTCACAGGAGTAGAAT (SEQ ID NO: 474) |
| hs02772569[St] | ATTTGTTCATCTTCCTCATCAGAAA (SEQ ID NO: 475) |
| hs02772570[St] | TTCTACAATAAAAAATGATGAGAAT (SEQ ID NO: 476) |
| hs02772571[St] | TTTTATTGTAGAATAAATGTAGAAT (SEQ ID NO: 477) |
| hs02772572[St] | TTCTCATCATTTTTTATTGTAGAAT (SEQ ID NO: 478) |
| hs02772573[St] | CAAAAGCCAGGGACCATGGGAGAAT (SEQ ID NO: 479) |
| hs02772574[St] | GTTCTGGGTACAGGGGTAAGAGAAA (SEQ ID NO: 480) |
| hs02772575[St] | AAAAGGAGCATGAAACAGGTAGAAT (SEQ ID NO: 481) |
| hs02772576[St] | CTTTTAACCAGACATCTAAGAGAAA (SEQ ID NO: 482) |
| hs02772577[St] | ATCATGCAAGTGACCTAAGGAGAAT (SEQ ID NO: 483) |
| hs02772578[St] | CCCAAACACAAAAATATTGAAGAAA (SEQ ID NO: 484) |
| hs02772579[St] | GGCTAATTTCACAAGGTTCAAGAAT (SEQ ID NO: 485) |

NmCas9

| | |
|---|---|
| hs02070240[Nm] | GAGGAAATTCTAATTTTGTAAAAAACTGGATT (SEQ ID NO: 486) |
| hs02070241[Nm] | AATTGAGGCCGTATATTGGATAACACAGGATT (SEQ ID NO: 487) |
| hs02070242[Nm] | TGCCTATGTGTGTGTGGGTGGGTGGCAGGATT (SEQ ID NO: 488) |
| hs02070243[Nm] | AATACTATAAGATCTGAGAGGAAAAAATGATT (SEQ ID NO: 489) |
| hs02070244[Nm] | AGATCTGAGAGGAAAAAATGATTGAAATGATT (SEQ ID NO: 490) |
| hs02070245[Nm] | AATGATTGAAATGATTGGAGCAGGAAATGATT (SEQ ID NO: 491) |
| hs02070246[Nm] | AGAAAAAGAGATTTGTAACAAACAATAGGATT (SEQ ID NO: 492) |
| hs02070247[Nm] | AAATATCAGGGTAGGTGGGAGAAAAAGAGATT (SEQ ID NO: 493) |
| hs02070248[Nm] | ATAGTAGAAGAAAAAAGAAATAGATGTAGATT (SEQ ID NO: 494) |
| hs02070249[Nm] | TCTATTCCATTCTTCACACATGAAATCTGATT (SEQ ID NO: 495) |
| hs02070250[Nm] | TTCATGAGACTAGAGGTCACGAAAATCAGATT (SEQ ID NO: 496) |
| hs02070251[Nm] | GGGGAGTCACATGGGAGTCACAGGGTAGGATT (SEQ ID NO: 497) |
| hs02070252[Nm] | CTTTGGCAAAAGCAGCAGAAAGCAAACTGATT (SEQ ID NO: 498) |
| hs02070253[Nm] | CAAAAGCAGCAGAAAGCAAACTGATTATGATT (SEQ ID NO: 499) |
| hs02070254[Nm] | CTTTCTGCTGCTTTTGCCAAAGAGAGGTGATT (SEQ ID NO: 500) |
| hs02070255[Nm] | TGAAAACGTTGTTCTGAGTAGCTTTCAGGATT (SEQ ID NO: 501) |
| hs02070256[Nm] | CATCAGAAATAGAGGCTTATGGATTTTGGATT (SEQ ID NO: 502) |
| hs02070257[Nm] | TCTTCCTCATCAGAAATAGAGGCTTATGGATT (SEQ ID NO: 503) |
| hs02070258[Nm] | CCTTTTACTGAGGACAGAACAAGCTCCTGATT (SEQ ID NO: 504) |
| hs02070259[Nm] | ACTACAAAGGGTCTGGTCCATATTCACAGATT (SEQ ID NO: 505) |
| hs02070260[Nm] | GGGCACTTAATGAGTGCTTCCCTCCTTTGATT (SEQ ID NO: 506) |
| hs02070261[Nm] | CTCTTAGATGTCTGGTTAAAAGAGGTTTGATT (SEQ ID NO: 507) |
| hs02070262[Nm] | TAGATCATTCTCCTTAGGTCACTTGCATGATT (SEQ ID NO: 508) |
| hs02070263[Nm] | ATTTATAGTGGCTGAATGACTTCTGAATGATT (SEQ ID NO: 509) |
| hs02070264[Nm] | ATATTTTTGTGTTTGGGGGGATTTGGCAGATT (SEQ ID NO: 510) |
| hs02070265[Nm] | TTTTCTTCAATATTTTTGTGTTTGGGGGGATT (SEQ ID NO: 511) |

SpCas9 (VRER)

| | |
|---|---|
| hg1223846[SpNGCG] | TCTGCTGCCTTCCACACTCTGGCG (SEQ ID NO: 512) |
| hg1223847[SpNGCG] | AGAAAGGAACATTTAGATAGAGCG (SEQ ID NO: 513) |
| hg1223848[SpNGCG] | TAGCTTTTGACAGTTTTTAAGGCG (SEQ ID NO: 514) |

Cpf1

| | |
|---|---|
| hs19346487[Cpf1] | TTTCTTAGTGATATTTTTCCTTTATTC (SEQ ID NO: 516) |
| hs19346488[Cpf1] | TTTGTGGTATTGGTCTGAATTACAATG (SEQ ID NO: 517) |
| hs19346489[Cpf1] | TTTTGTGGTATTGGTCTGAATTACAAT (SEQ ID NO: 518) |
| hs19346490[Cpf1] | TTTATATAACATCTCCTTAAAACTCAC (SEQ ID NO: 519) |
| hs19346491[Cpf1] | TTTAAGGAGATGTTATATAAAGTTAAG (SEQ ID NO: 520) |
| hs19346492[Cpf1] | TTTTAAGGAGATGTTATATAAAGTTAA (SEQ ID NO: 521) |
| hs19346493[Cpf1] | TTTGTAAAAAACTGGATTGTGAGTTTT (SEQ ID NO: 522) |
| hs19346494[Cpf1] | TTTTGTAAAAAACTGGATTGTGAGTTT (SEQ ID NO: 523) |
| hs19346495[Cpf1] | TTTCTGAGTATTAGCTTCTGCCACTAA (SEQ ID NO: 524) |
| hs19346496[Cpf1] | TTTAGTGGCAGAAGCTAATACTCAGAA (SEQ ID NO: 525) |
| hs19346497[Cpf1] | TTTTGGCCAAAACTCTTAGAATACTCA (SEQ ID NO: 526) |
| hs19346498[Cpf1] | TTTGGCCAAAACTCTTAGAATACTCAA (SEQ ID NO: 527) |
| hs19346499[Cpf1] | TTTGGCCAAAATGAATGTACAGCTTTA (SEQ ID NO: 528) |
| hs19346500[Cpf1] | TTTTGGCCAAAATGAATGTACAGCTTT (SEQ ID NO: 529) |
| hs19346501[Cpf1] | TTTTGCAGCCTTCACAAACTAGTGCTT (SEQ ID NO: 530) |
| hs19346502[Cpf1] | TTTGCAGCCTTCACAAACTAGTGCTTA (SEQ ID NO: 531) |
| hs19346503[Cpf1] | TTTGTGAAGGCTGCAAAACTCTTGAGT (SEQ ID NO: 532) |
| hs19346504[Cpf1] | TTTACAGTGAAAAGAAATATAAAATTC (SEQ ID NO: 533) |
| hs19346505[Cpf1] | TTTCACTGTAAACATCATCATCTGTCA (SEQ ID NO: 534) |
| hs19346506[Cpf1] | TTTTCACTGTAAACATCATCATCTGTC (SEQ ID NO: 535) |
| hs19346507[Cpf1] | TTTCTTTTCACTGTAAACATCATCATC (SEQ ID NO: 536) |
| hs19346508[Cpf1] | TTTATATTTCTTTTCACTGTAAACATC (SEQ ID NO: 537) |
| hs19346509[Cpf1] | TTTTATATTTCTTTTCACTGTAAACAT (SEQ ID NO: 538) |
| hs19346510[Cpf1] | TTTCGCCAGAGTGTGGAAGGCAGCAGA (SEQ ID NO: 539) |
| hs19346511[Cpf1] | TTTACTTGCTTATTATCTGCTGCCTTC (SEQ ID NO: 540) |
| hs19346512[Cpf1] | TTTGTTTACTTGCTTATTATCTGCTGC (SEQ ID NO: 541) |
| hs19346513[Cpf1] | TTTGTTTGTTTACTTGCTTATTATCTG (SEQ ID NO: 542) |
| hs19346514[Cpf1] | TTTTGTTTGTTTACTTGCTTATTATCT (SEQ ID NO: 543) |
| hs19346515[Cpf1] | TTTTTGTTTGTTTACTTGCTTATTATC (SEQ ID NO: 544) |
| hs19346516[Cpf1] | TTTAGTAGACTTTTTGTTTGTTTACTT (SEQ ID NO: 545) |
| hs19346517[Cpf1] | TTTTAGTAGACTTTTTGTTTGTTTACT (SEQ ID NO: 546 |
| hs19346518[Cpf1] | TTTTTAGTAGACTTTTTGTTTGTTTAC (SEQ ID NO: 547) |
| hs19346519[Cpf1] | TTTAGAAAGAAAATAAAAACAGAGTAC (SEQ ID NO: 548) |
| hs19346520[Cpf1] | TTTCTATTATTCACTGGCTCAGTACTC (SEQ ID NO: 549) |
| hs19346521[Cpf1] | TTTAGATAGAGCGGTTGAAGGATGTCT (SEQ ID NO: 550) |
| hs19346522[Cpf1] | TTTGGTGGTTAAGACATCCTTCAACCG (SEQ ID NO: 551) |
| hs19346523[Cpf1] | TTTAAGCTGAACTGTGAGGTACGAAGT (SEQ ID NO: 552) |
| hs19346524[Cpf1] | TTTATTAGTAAACCCAGATGCTCCAAA (SEQ ID NO: 553) |
| hs19346525[Cpf1] | TTTACTAATAAATACTTCGTACCTCAC (SEQ ID NO: 554) |
| hs19346526[Cpf1] | TTTGGAGCATCTGGGTTTACTAATAAA (SEQ ID NO: 555) |
| hs19346527[Cpf1] | TTTCTTCCTTTGGAGCATCTGGGTTTA (SEQ ID NO: 556) |
| hs19346528[Cpf1] | TTTGGAATACTATAAGATCTGAGAGGA (SEQ ID NO: 557) |
| hs19346529[Cpf1] | TTTCCTCTCAGATCTTATAGTATTCCA (SEQ ID NO: 558) |
| hs19346530[Cpf1] | TTTTCCTCTCAGATCTTATAGTATTCC (SEQ ID NO: 559) |
| hs19346531[Cpf1] | TTTTTCCTCTCAGATCTTATAGTATTC (SEQ ID NO: 560) |
| hs19346532[Cpf1] | TTTTTTCCTCTCAGATCTTATAGTATT (SEQ ID NO: 561) |
| hs19346533[Cpf1] | TTTCAATCATTTTTTCCTCTCAGATCT (SEQ ID NO: 562) |
| hs19346534[Cpf1] | TTTCCTGCTCCAATCATTTCAATCATT (SEQ ID NO: 563) |
| hs19346535[Cpf1] | TTTTTTAAGTAAAGTACATAATTATAG (SEQ ID NO: 564) |
| hs19346536[Cpf1] | TTTTTAAGTAAAGTACATAATTATAGT (SEQ ID NO: 565) |
| hs19346537[Cpf1] | TTTTAAGTAAAGTACATAATTATAGTT (SEQ ID NO: 566) |
| hs19346538[Cpf1] | TTTAAGTAAAGTACATAATTATAGTTT (SEQ ID NO: 567) |
| hs19346539[Cpf1] | TTTTATATACTATGAATACACAGGAGG (SEQ ID NO: 568) |
| hs19346540[Cpf1] | TTTATATACTATGAATACACAGGAGGG (SEQ ID NO: 569) |
| hs19346541[Cpf1] | TTTCCCTCCTGTGTATTCATAGTATAT (SEQ ID NO: 570) |
| hs19346542[Cpf1] | TTTTCCCTCCTGTGTATTCATAGTATA (SEQ ID NO: 571) |
| hs19346543[Cpf1] | TTTTAAATATCTTTCTATCCATTTTTT (SEQ ID NO: 572) |
| hs19346544[Cpf1] | TTTAAATATCTTTCTATCCATTTTTTT (SEQ ID NO: 573) |
| hs19346545[Cpf1] | TTTAAAACACCCTCAGCCTCCTGTTTT (SEQ ID NO: 574) |
| hs19346546[Cpf1] | TTTCTATCCATTTTTTTCTTCCTTTCA (SEQ ID NO: 575) |
| hs19346547[Cpf1] | TTTTTTTCTTCCTTTCATCCTTTTTCA (SEQ ID NO: 576) |
| hs19346548[Cpf1] | TTTTTTCTTCCTTTCATCCTTTTTCAC (SEQ ID NO: 577) |
| hs19346549[Cpf1] | TTTTTCTTCCTTTCATCCTTTTTCACA (SEQ ID NO: 578) |
| hs19346550[Cpf1] | TTTTCTTCCTTTCATCCTTTTTCACAG (SEQ ID NO: 579) |
| hs19346551[Cpf1] | TTTCTTCCTTTCATCCTTTTTCACAGG (SEQ ID NO: 580) |
| hs19346552[Cpf1] | TTTCATCCTTTTTCACAGGATAATCCT (SEQ ID NO: 581) |
| hs19346553[Cpf1] | TTTTTCACAGGATAATCCTATTGTTTG (SEQ ID NO: 582) |
| hs19346554[Cpf1] | TTTTCACAGGATAATCCTATTGTTTGT (SEQ ID NO: 583) |
| hs19346555[Cpf1] | TTTCACAGGATAATCCTATTGTTTGTT (SEQ ID NO: 584) |
| hs19346556[Cpf1] | TTTGTTACAAATCTCTTTTTCTCCCAC (SEQ ID NO: 585) |
| hs19346557[Cpf1] | TTTGTAACAAACAATAGGATTATCCTG (SEQ ID NO: 586) |
| hs19346558[Cpf1] | TTTTTCTCCCACCTACCCTGATATTTT (SEQ ID NO: 587) |
| hs19346559[Cpf1] | TTTTCTCCCACCTACCCTGATATTTTG (SEQ ID NO: 588) |
| hs19346560[Cpf1] | TTTCTCCCACCTACCCTGATATTTTGC (SEQ ID NO: 589) |
| hs19346561[Cpf1] | TTTTGCTATTTCCCTTATTACCACTTT (SEQ ID NO: 590) |
| hs19346562[Cpf1] | TTTGCTATTTCCCTTATTACCACTTTC (SEQ ID NO: 591) |
| hs19346563[Cpf1] | TTTCCCTTATTACCACTTTCATCTCTT (SEQ ID NO: 592) |
| hs19346564[Cpf1] | TTTCATCTCTTATCTTCATCACTCTCT (SEQ ID NO: 593) |
| hs19346565[Cpf1] | TTTAGAGTTACTAGGGAGAGAGTGATG (SEQ ID NO: 594) |
| hs19346566[Cpf1] | TTTTCTTTCCCTCCTCCTGAAACTTCA (SEQ ID NO: 595) |
| hs19346567[Cpf1] | TTTCTTTCCCTCCTCCTGAAACTTCAC (SEQ ID NO: 596) |
| hs19346568[Cpf1] | TTTCCCTCCTCCTGAAACTTCACTGGT (SEQ ID NO: 597) |
| hs19346569[Cpf1] | TTTCAGGAGGAGGGAAAGAAAATTTAG (SEQ ID NO: 598) |
| hs19346570[Cpf1] | TTTAAAGGAGACCAGTGAAGTTTCAGG (SEQ ID NO: 599) |
| hs19346571[Cpf1] | TTTTAAAGGAGACCAGTGAAGTTTCAG (SEQ ID NO: 600) |
| hs19346572[Cpf1] | TTTTTAAAGGAGACCAGTGAAGTTTCA (SEQ ID NO: 601) |
| hs19346573[Cpf1] | TTTTTTAAAGGAGACCAGTGAAGTTTC (SEQ ID NO: 602) |
| hs19346574[Cpf1] | TTTTTTTAAAGGAGACCAGTGAAGTTT (SEQ ID NO: 603) |
| hs19346575[Cpf1] | TTTTTTTTAAAGGAGACCAGTGAAGTT (SEQ ID NO: 604) |
| hs19346576[Cpf1] | TTTTTTTTTAAAGGAGACCAGTGAAGT (SEQ ID NO: 605) |
| hs19346577[Cpf1] | TTTTTTTTTTAAAGGAGACCAGTGAAG (SEQ ID NO: 606) |
| hs19346578[Cpf1] | TTTTTTTTTTTAAAGGAGACCAGTGAA (SEQ ID NO: 607) |
| hs19346579[Cpf1] | TTTTTTTTTTTTAAAGGAGACCAGTGA (SEQ ID NO: 608) |
| hs19346580[Cpf1] | TTTTTTTTTTTTTAAAGGAGACCAGTG (SEQ ID NO: 609) |
| hs19346581[Cpf1] | TTTCTTTTTTCTTCTACTATCAAATTT (SEQ ID NO: 610) |
| hs19346582[Cpf1] | TTTTTTCTTCTACTATCAAATTTCAAG (SEQ ID NO: 611) |
| hs19346583[Cpf1] | TTTTTCTTCTACTATCAAATTTCAAGT (SEQ ID NO: 612) |
| hs19346584[Cpf1] | TTTTCTTCTACTATCAAATTTCAAGTT (SEQ ID NO: 613) |
| hs19346585[Cpf1] | TTTCTTCTACTATCAAATTTCAAGTTT (SEQ ID NO: 614) |
| hs19346586[Cpf1] | TTTGATAGTAGAAGAAAAAAGAAATAG (SEQ ID NO: 615) |
| hs19346587[Cpf1] | TTTCAAGTTTTTCTTTTGCTTTATTAC (SEQ ID NO: 616) |
| hs19346588[Cpf1] | TTTTTCTTTTGCTTTATTACCCATCCA (SEQ ID NO: 617) |
| hs19346589[Cpf1] | TTTTCTTTTGCTTTATTACCCATCCAG (SEQ ID NO: 618) |
| hs19346590[Cpf1] | TTTCTTTTGCTTTATTACCCATCCAGT (SEQ ID NO: 619) |
| hs19346591[Cpf1] | TTTTGCTTTATTACCCATCCAGTTTTT (SEQ ID NO: 620) |
| hs19346592[Cpf1] | TTTGCTTTATTACCCATCCAGTTTTTG (SEQ ID NO: 621) |
| hs19346593[Cpf1] | TTTATTACCCATCCAGTTTTTGTATTT (SEQ ID NO: 622) |
| hs19346594[Cpf1] | TTTTTGTATTTCCTTATTATCTATTCC (SEQ ID NO: 623) |
| hs19346595[Cpf1] | TTTTGTATTTCCTTATTATCTATTCCA (SEQ ID NO: 624) |
| hs19346596[Cpf1] | TTTGTATTTCCTTATTATCTATTCCAT (SEQ ID NO: 625) |
| hs19346597[Cpf1] | TTTCCTTATTATCTATTCCATTCTTCA (SEQ ID NO: 626) |
| hs19346598[Cpf1] | TTTCATGTGTGAAGAATGGAATAGATA (SEQ ID NO: 627) |
| hs19346599[Cpf1] | TTTTCGTGACCTCTAGTCTCATGAACC (SEQ ID NO: 628) |
| hs19346600[Cpf1] | TTTCGTGACCTCTAGTCTCATGAACCT (SEQ ID NO: 629) |
| hs19346601[Cpf1] | TTTACCTCTCAAAAAGCAACAATAATT (SEQ ID NO: 630) |
| hs19346602[Cpf1] | TTTGAGAGGTAAAGGTTCATGAGACTA (SEQ ID NO: 631) |
| hs19346603[Cpf1] | TTTTGAGAGGTAAAGGTTCATGAGACT (SEQ ID NO: 632) |
| hs19346604[Cpf1] | TTTTTGAGAGGTAAAGGTTCATGAGAC (SEQ ID NO: 633) |
| hs19346605[Cpf1] | TTTCCTAAGCACCATATTTGACACCAC (SEQ ID NO: 634) |
| hs19346606[Cpf1] | TTTGACACCACATGCACTGTGGCTCTC (SEQ ID NO: 635) |
| hs19346607[Cpf1] | TTTTACCCTTCAGGTAACCGGTAGCTT (SEQ ID NO: 636) |
| hs19346608[Cpf1] | TTTACCCTTCAGGTAACCGGTAGCTTT (SEQ ID NO: 637) |
| hs19346609[Cpf1] | TTTTGACAGTTTTTAAGGCGGGGAGTC (SEQ ID NO: 638) |
| hs19346610[Cpf1] | TTTGACAGTTTTTAAGGCGGGGAGTCA (SEQ ID NO: 639) |
| hs19346611[Cpf1] | TTTTTAAGGCGGGGAGTCACATGGGAG (SEQ ID NO: 640) |
| hs19346612[Cpf1] | TTTTAAGGCGGGGAGTCACATGGGAGT (SEQ ID NO: 641) |
| hs19346613[Cpf1] | TTTAAGGCGGGGAGTCACATGGGAGTC (SEQ ID NO: 642) |
| hs19346614[Cpf1] | TTTAGAATGATCATTCTTGTGGCAGTA (SEQ ID NO: 643) |
| hs19346615[Cpf1] | TTTATTGTGGCAGCCATTATTCCTGTC (SEQ ID NO: 644) |
| hs19346616[Cpf1] | TTTATTATCTTTATTGTGGCAGCCATT (SEQ ID NO: 645) |
| hs19346617[Cpf1] | TTTTATTATCTTTATTGTGGCAGCCAT (SEQ ID NO: 646) |
| hs19346618[Cpf1] | TTTTTATTATCTTTATTGTGGCAGCCA (SEQ ID NO: 647) |
| hs19346619[Cpf1] | TTTTTTATTATCTTTATTGTGGCAGCC (SEQ ID NO: 648) |
| hs19346620[Cpf1] | TTTATTTTTTATTATCTTTATTGTGGC (SEQ ID NO: 649) |
| hs19346621[Cpf1] | TTTTATTTTTTATTATCTTTATTGTGG (SEQ ID NO: 650) |
| hs19346622[Cpf1] | TTTAGGAAAGATGAAAAATACTCTTTG (SEQ ID NO: 651) |
| hs19346623[Cpf1] | TTTGGGAAAATGATAACGTTAAACATC (SEQ ID NO: 652) |
| hs19346624[Cpf1] | TTTCCCAAAGAGTATTTTTCATCTTTC (SEQ ID NO: 653) |
| hs19346625[Cpf1] | TTTTCCCAAAGAGTATTTTTCATCTTT (SEQ ID NO: 654) |
| hs19346626[Cpf1] | TTTAACGTTATCATTTTCCCAAAGAGT (SEQ ID NO: 655) |
| hs19346627[Cpf1] | TTTCTAATGCTGGAGAGGATAGGACAG (SEQ ID NO: 656) |
| hs19346628[Cpf1] | TTTCCATTCTCCATGTCCTCTGTCCTA (SEQ ID NO: 657) |
| hs19346629[Cpf1] | TTTTCCATTCTCCATGTCCTCTGTCCT (SEQ ID NO: 658) |
| hs19346630[Cpf1] | TTTTTCCATTCTCCATGTCCTCTGTCC (SEQ ID NO: 659) |
| hs19346631[Cpf1] | TTTTTTCCATTCTCCATGTCCTCTGTC (SEQ ID NO: 660) |
| hs19346632[Cpf1] | TTTTTTTCCATTCTCCATGTCCTCTGT (SEQ ID NO: 661) |
| hs19346633[Cpf1] | TTTATTTTTTTCCATTCTCCATGTCCT (SEQ ID NO: 662) |
| hs19346634[Cpf1] | TTTAGTGTGGTGATAATTTATTTTTTT (SEQ ID NO: 663) |
| hs19346635[Cpf1] | TTTTCTATAAAAAGTAACATAATCACC (SEQ ID NO: 664) |
| hs19346636[Cpf1] | TTTCTATAAAAAGTAACATAATCACCT (SEQ ID NO: 665) |
| hs19346637[Cpf1] | TTTATAGAAAATTATGCCTATTTAGTG (SEQ ID NO: 666) |
| hs19346638[Cpf1] | TTTTATAGAAAATTATGCCTATTTAGT (SEQ ID NO: 667) |
| hs19346639[Cpf1] | TTTTTATAGAAAATTATGCCTATTTAG (SEQ ID NO: 668) |
| hs19346640[Cpf1] | TTTGGCAAAAGCAGCAGAAAGCAAACT (SEQ ID NO: 669) |
| hs19346641[Cpf1] | TTTGCCAAAGAGAGGTGATTATGTTAC (SEQ ID NO: 670) |
| hs19346642[Cpf1] | TTTTGCCAAAGAGAGGTGATTATGTTA (SEQ ID NO: 671) |
| hs19346643[Cpf1] | TTTCTGCTGCTTTTGCCAAAGAGAGGT (SEQ ID NO: 672) |
| hs19346644[Cpf1] | TTTGCTTTCTGCTGCTTTTGCCAAAGA (SEQ ID NO: 673) |
| hs19346645[Cpf1] | TTTAGAAGTAATCATAATCAGTTTGCT (SEQ ID NO: 674) |
| hs19346646[Cpf1] | TTTATCAATATTATTTAGAAGTAATCA (SEQ ID NO: 675) |
| hs19346647[Cpf1] | TTTAGTAGGAATCCTGAAAGCTACTCA (SEQ ID NO: 676) |
| hs19346648[Cpf1] | TTTCAGGATTCCTACTAAATTATGAGT (SEQ ID NO: 677) |
| hs19346649[Cpf1] | TTTTCATTTACTGAATGTGTCTCTATG (SEQ ID NO: 678) |
| hs19346650[Cpf1] | TTTCATTTACTGAATGTGTCTCTATGA (SEQ ID NO: 679) |
| hs19346651[Cpf1] | TTTACTGAATGTGTCTCTATGAGCCAG (SEQ ID NO: 680) |
| hs19346652[Cpf1] | TTTGCTGGCTCATAGAGACACATTCAG (SEQ ID NO: 681) |
| hs19346653[Cpf1] | TTTTGCTGGCTCATAGAGACACATTCA (SEQ ID NO: 682) |
| hs19346654[Cpf1] | TTTTGAGCTAATAGAATTAGATCTTAT (SEQ ID NO: 683) |
| hs19346655[Cpf1] | TTTGAGCTAATAGAATTAGATCTTATT (SEQ ID NO: 684) |
| hs19346656[Cpf1] | TTTCACAGGAGTAGAATAAGATCTAAT (SEQ ID NO: 685) |
| hs19346657[Cpf1] | TTTGGATTATTCTTATCTAAGATCCTT (SEQ ID NO: 686) |
| hs19346658[Cpf1] | TTTTGGATTATTCTTATCTAAGATCCT (SEQ ID NO: 687) |
| hs19346659[Cpf1] | TTTCTGATGAGGAAGATGAACAAATCA (SEQ ID NO: 688) |
| hs19346660[Cpf1] | TTTGTTCATCTTCCTCATCAGAAATAG (SEQ ID NO: 689) |
| hs19346661[Cpf1] | TTTACTGAGGACAGAACAAGCTCCTGA (SEQ ID NO: 690) |
| hs19346662[Cpf1] | TTTTACTGAGGACAGAACAAGCTCCTG (SEQ ID NO: 691) |
| hs19346663[Cpf1] | TTTGTAGTTATCTTACAGCCACTTGAA (SEQ ID NO: 692) |
| hs19346664[Cpf1] | TTTATTCTACAATAAAAAATGATGAGA (SEQ ID NO: 693) |
| hs19346665[Cpf1] | TTTATTGTAGAATAAATGTAGAATTTT (SEQ ID NO: 694) |
| hs19346666[Cpf1] | TTTTATTGTAGAATAAATGTAGAATTT (SEQ ID NO: 695) |
| hs19346667[Cpf1] | TTTTTATTGTAGAATAAATGTAGAATT (SEQ ID NO: 696) |
| hs19346668[Cpf1] | TTTTTTATTGTAGAATAAATGTAGAAT (SEQ ID NO: 697) |
| hs19346669[Cpf1] | TTTACAGAGTGCATCCATGGTCCAGGA (SEQ ID NO: 698) |
| hs19346670[Cpf1] | TTTCAGCTACTGTTGGCTACATCCATT (SEQ ID NO: 699) |
| hs19346671[Cpf1] | TTTGTTCCTTGGAATGGATGTAGCCAA (SEQ ID NO: 700) |
| hs19346672[Cpf1] | TTTTGTTCCTTGGAATGGATGTAGCCA (SEQ ID NO: 701) |
| hs19346673[Cpf1] | TTTGTTCTGGGTACAGGGGTAAGAGAA (SEQ ID NO: 702) |
| hs19346674[Cpf1] | TTTCTCTTACCCCTGTACCCAGAACAA (SEQ ID NO: 703) |
| hs19346675[Cpf1] | TTTGATTACAATATTCTACCTGTTTCA (SEQ ID NO: 704) |
| hs19346676[Cpf1] | TTTCATGCTCCTTTTTCTCTTAGATGT (SEQ ID NO: 705) |
| hs19346677[Cpf1] | TTTTTCTCTTAGATGTCTGGTTAAAAG (SEQ ID NO: 706) |
| hs19346678[Cpf1] | TTTTCTCTTAGATGTCTGGTTAAAAGA (SEQ ID NO: 707) |
| hs19346679[Cpf1] | TTTCTCTTAGATGTCTGGTTAAAAGAG (SEQ ID NO: 708) |
| hs19346680[Cpf1] | TTTAACCAGACATCTAAGAGAAAAAGG (SEQ ID NO: 709) |
| hs19346681[Cpf1] | TTTTAACCAGACATCTAAGAGAAAAAG (SEQ ID NO: 710) |
| hs19346682[Cpf1] | TTTGATTCAGCTAAATCATGCAAGTGA (SEQ ID NO: 711) |
| hs19346683[Cpf1] | TTTAGCTGAATCAAACCTCTTTTAACC (SEQ ID NO: 712) |
| hs19346684[Cpf1] | TTTATAGTGGCTGAATGACTTCTGAAT (SEQ ID NO: 713) |
| hs19346685[Cpf1] | TTTGTTCCCACATAACACACACACACA (SEQ ID NO: 714) |
| hs19346686[Cpf1] | TTTTGTTCCCACATAACACACACACAC (SEQ ID NO: 715) |
| hs19346687[Cpf1] | TTTATAACTAAACTGAATGAATAATAT (SEQ ID NO: 716) |
| hs19346688[Cpf1] | TTTAGTTATAAATATTTTGTTCCCACA (SEQ ID NO: 717) |
| hs19346689[Cpf1] | TTTAACAGAGATATAATTATCCATATT (SEQ ID NO: 718) |
| hs19346690[Cpf1] | TTTATATAAATGCAGGCAAACTTTAAT (SEQ ID NO: 719) |
| hs19346691[Cpf1] | TTTATATAAATTTAACAGAGATATAAT (SEQ ID NO: 720) |
| hs19346692[Cpf1] | TTTGCCTGCATTTATATAAATTTAACA (SEQ ID NO: 721) |
| hs19346693[Cpf1] | TTTAATTAAAATCTGCCAAATCCCCCC (SEQ ID NO: 722) |
| hs19346694[Cpf1] | TTTAATTAAAGTTTGCCTGCATTTATA (SEQ ID NO: 723) |
| hs19346695[Cpf1] | TTTTAATTAAAGTTTGCCTGCATTTAT (SEQ ID NO: 724) |
| hs19346696[Cpf1] | TTTGGCAGATTTTAATTAAAGTTTGCC (SEQ ID NO: 725) |
| hs19346697[Cpf1] | TTTGGGGGGATTTGGCAGATTTTAATT (SEQ ID NO: 726) |
| hs19346698[Cpf1] | TTTGTGTTTGGGGGGATTTGGCAGATT (SEQ ID NO: 727) |
| hs19346699[Cpf1] | TTTTGTGTTTGGGGGGATTTGGCAGAT (SEQ ID NO: 728) |
| hs19346700[Cpf1] | TTTTTGTGTTTGGGGGGATTTGGCAGA (SEQ ID NO: 729) |
| hs19346701[Cpf1] | TTTCTTCAATATTTTTGTGTTTGGGGG (SEQ ID NO: 730 |
| hs19346702[Cpf1] | TTTTCTTCAATATTTTTGTGTTTGGGG (SEQ ID NO: 731) |
| hs19346703[Cpf1] | TTTTTCTTCAATATTTTTGTGTTTGGG (SEQ ID NO: 732) |
| hs19346704[Cpf1] | TTTCACAAGGTTCAAGAATCACACAAA (SEQ ID NO: 733) |
| hs19346705[Cpf1] | TTTGTGTGATTCTTGAACCTTGTGAAA (SEQ ID NO: 734) |

TABLE 5
LCA10 targets

| ID | sequence |
|---|---|
| SaCas9 | CACCTGGCCCCAGTTGTAATTGTGAGT (SEQ ID |
| | NO: 735) |
| SpCas9(VQR) | CCTGGCCCCAGTTGTAATTGTGA (SEQ ID NO: |
| | 736) |
| SpCas9(EQR) | CCTGGCCCCAGTTGTAATTGTGAG (SEQ ID NO: |
| | 737) |
| hs028893108* | GAGATACTCACAATTACAACTGG (SEQ ID NO: |
| | 738) |

TABLE 8
MAP3K12 (DLK) exonic target sites
SpCas9

| grID | sequence | exon |
|---|---|---|
| hs027563580 | CGGCCAGGCTCTCACCTCGGGGG | 1 |
| | (SEQ ID NO: 788) | |
| hs027563586 | CTCTCACCTCGGGGGCTCCGCGG | 1 |
| | (SEQ ID NO: 789) | |
| hs027563601 | GCTGCCCTCCACCGGACCCGGGG | 1 |
| | (SEQ ID NO: 790) | |
| hs027563599 | CAGCGCCGCCTTTTGTGCTGCGG | 1 |
| | (SEQ ID NO: 791) | |
| hs027563587 | AGCCTGGCCGAGTTGTGGGGGGG | 1 |
| | (SEQ ID NO: 792) | |
| hs027563588 | GAGCCTGGCCGAGTTGTGGGGGG | 1 |
| | (SEQ ID NO: 793) | |
| hs027563610 | CATCGCCTGTGACAGCGCCCCGG | 1 |
| | (SEQ ID NO: 794) | |
| hs027563611 | TCGTGCGGTGTCCGGATGCACGG | 1 |
| | (SEQ ID NO: 795) | |
| hs027563595 | CGCGGCCGCAGCACAAAAGGCGG | 1 |
| | (SEQ ID NO: 796) | |
| hs027563596 | CTGCGGCCGCGGAGCCCCCGAGG | 1 |
| | (SEQ ID NO: 797) | |
| hs027563623 | GCAGGGACGGGGCTAGGGGTCGG | 1 |
| | (SEQ ID NO: 798) | |
| hs027563598 | GCGCTGCTGCAGCTCCGCTCCGG | 1 |
| | (SEQ ID NO: 799) | |
| hs027563597 | CGCCTTTTGTGCTGCGGCCGCGG | 1 |
| | (SEQ ID NO: 801) | |
| hs027562294 | AGGGTGTTCGGGTCTCATGGAGG | 2 |
| | (SEQ ID NO: 802) | |
| hs027562271 | TCTCGAAGTACACACTGGGTAGG | 2 |
| | (SEQ ID NO: 803) | |
| hs027562307 | ACCATCATACCAGGGGCCAGAGG | 2 |
| | (SEQ ID NO: 804) | |
| hs027562245 | ACTGTTGGCAAAAGGCTCAGGGG | 2 |
| | (SEQ ID NO: 8058) | |
| hs027562237 | CTACATGAGCAGGATGCAGGGGG | 2 |
| | (SEQ ID NO: 806) | |
| hs027562290 | CTTTGTGTCTACCCTAAGTGAGG | 2 |
| | (SEQ ID NO: 807) | |
| hs027562267 | CCACATCTCGAAGTACACACTGG | 2 |
| | (SEQ ID NO: 808) | |
| hs027562268 | CACATCTCGAAGTACACACTGGG | 2 |
| | (SEQ ID NO: 809) | |
| hs027562210 | TGCCAATCATGGTCCAGACAGGG | 2 |
| | (SEQ ID NO: 810) | |
| hs027562274 | CTTCGAGATGTGGTACCCCTTGG | 2 |
| | (SEQ ID NO: 811) | |
| hs027562224 | ATCCAGAGTTCGAGCTGACGAGG | 2 |
| | (SEQ ID NO: 812) | |
| hs027562246 | CTGTTGGCAAAAGGCTCAGGGGG | 2 |
| | (SEQ ID NO: 813) | |
| hs027562211 | TCATGGTCCAGACAGGGCGCAGG | 2 |
| | (SEQ ID NO: 814) | |
| hs027562259 | CCCCCACCCTGCCCACCAAGGGG | 2 |
| | (SEQ ID NO: 815) | |
| hs027562280 | GAGTGCAGTCAGAAGTGTCTGGG | 2 |
| | (SEQ ID NO: 816) | |
| hs027562282 | GTCTGGGTCCAGCTTGCGCATGG | 2 |
| | (SEQ ID NO: 817) | |
| hs027562278 | GTAGGCGTCAGGTCCTTCTCGGG | 2 |
| | (SEQ ID NO: 818) | |
| hs027562238 | GCTACATGAGCAGGATGCAGGGG | 2 |
| | (SEQ ID NO: 819) | |
| hs027562203 | CTGCTGCTGCTTGTGCTCAGTGG | 2 |
| | (SEQ ID NO: 820) | |
| hs027562302 | CCTGGTATGATGGTGAACACTGG | 2 |
| | (SEQ ID NO: 821) | |
| hs027562256 | GGGCCCAGCCCCTCCCCAGGTGG | 2 |
| | (SEQ ID NO: 822) | |
| hs027562295 | ACACCCTCTCCTTCCTTTGGGGG | 2 |
| | (SEQ ID NO: 823) | |
| hs027562239 | AGCTACATGAGCAGGATGCAGGG | 2 |
| | (SEQ ID NO: 824) | |
| hs027562247 | TTGGCAAAAGGCTCAGGGGGCGG | 2 |
| | (SEQ ID NO: 825) | |
| hs027562205 | ACAAGCAGCAGCAGGAAGGTAGG | 2 |
| | (SEQ ID NO: 826) | |
| hs027562209 | TTGCCAATCATGGTCCAGACAGG | 2 |
| | (SEQ ID NO: 827) | |
| hs027562284 | GCGCATGGATGCCTCACTTAGGG | 2 |
| | (SEQ ID NO: 828) | |
| hs027562220 | CTGCAGTGCCAGAGTGGCAGTGG | 2 |
| | (SEQ ID NO: 829) | |
| hs027562258 | TCCCCCACCCTGCCCACCAAGGG | 2 |
| | (SEQ ID NO: 830) | |
| hs027562218 | AGTGGCAGTGGCTTCCTTGAGGG | 2 |
| | (SEQ ID NO: 831) | |
| hs027562221 | CACCTCGTCAGCTCGAACTCTGG | 2 |
| | (SEQ ID NO: 832) | |
| hs027562253 | GCGGCTCTCCACCTGGGGAGGGG | 2 |
| | (SEQ ID NO: 833) | |
| hs027562124 | GGTCCAGGATTTCCTCAAAGGGG | 3 |
| | (SEQ ID NO: 834) | |
| hs027562099 | TCACTTTCAAGTGAGGTCCAGGG | 3 |
| | (SEQ ID NO: 835) | |
| hs027562131 | GGAAATCCTGGACCTGCAGTGGG | 3 |
| | (SEQ ID NO: 836) | |
| hs027562104 | TCACAGCCACCTCCTCCCCGTGG | 3 |
| | (SEQ ID NO: 837) | |
| hs027562101 | AACATCATCACTTTCAAGTGAGG | 3 |
| | (SEQ ID NO: 838) | |
| hs027562130 | CCTCAAAGGGGACCTCCCAAAGG | 3 |
| | (SEQ ID NO: 839) | |
| hs027562112 | CTTCCTGGGGCGCTTCCACGGGG | 3 |
| | (SEQ ID NO: 840) | |
| hs027562111 | CCTGGGGCGCTTCCACGGGGAGG | 3 |
| | (SEQ ID NO: 841) | |
| hs027562102 | CTTTCGCAAGTGCTTGATGTCGG | 3 |
| | (SEQ ID NO: 842) | |
| hs027562132 | AGGAAATCCTGGACCTGCAGTGG | 3 |
| | (SEQ ID NO: 843) | |
| hs027562096 | TCAAGTGAGGTCCAGGGCAGGGG | 3 |
| | (SEQ ID NO: 844) | |
| hs027562103 | GCTTGATGTCGGTTTCTTTGAGG | 3 |
| | (SEQ ID NO: 845) | |
| hs027562108 | GCGCTTCCACGGGGAGGAGGTGG | 3 |
| | (SEQ ID NO: 846) | |
| hs027562006 | TGGGTGACTTGAGATCCCTGTGG | 4 |
| | (SEQ ID NO: 847) | |
| hs027562034 | CCTGGGCGCAGAACTCCATGAGG | 4 |
| | (SEQ ID NO: 848) | |
| hs027562011 | CCTGCACAAGATTATCCACAGGG | 4 |
| | (SEQ ID NO: 849) | |
| hs027562023 | GTAAGGAGGGGGTGACAGGGCGG | 4 |
| | (SEQ ID NO: 850) | |
| hs027562017 | ACCAGTCAACCAGTAAGGAGGGG | 4 |
| | (SEQ ID NO: 851) | |
| hs027562018 | CCAGTCAACCAGTAAGGAGGGGG | 4 |
| | (SEQ ID NO: 852) | |
| hs027562015 | GGACCAGTCAACCAGTAAGGAGG | 4 |
| | (SEQ ID NO: 853) | |
| hs027562037 | TCCATGAGGATGCAGTAGCAGGG | 4 |
| | (SEQ ID NO: 854) | |
| hs027562012 | ACCTGCACAAGATTATCCACAGG | 4 |
| | (SEQ ID NO: 855) | |
| hs027562016 | GACCAGTCAACCAGTAAGGAGGG | 4 |
| | (SEQ ID NO: 856) | |
| hs027562010 | GGATAATCTTGTGCAGGTGCAGG | 4 |
| | (SEQ ID NO: 857) | |
| hs027562042 | TCCCTGCTACTGCATCCTCATGG | 4 |
| | (SEQ ID NO: 858) | |
| hs027562033 | GCCAGCTGTATGAGGTACTGCGG | 4 |
| | (SEQ ID NO: 859) | |
| hs027562007 | CCCAAGTGAGTAAGCAAGCAGGG | 4 |
| | (SEQ ID NO: 860) | |
| hs027562032 | CCAGCTGTATGAGGTACTGCGGG | 4 |
| | (SEQ ID NO: 861) | |
| hs027562041 | GATGCAGTAGCAGGGAGCCTGGG | 4 |
| | (SEQ ID NO: 862) | |
| hs027562030 | TACCTCATACAGCTGGCCCTGGG | 4 |
| | (SEQ ID NO: 863) | |
| hs027562024 | TTACTGGTTGACTGGTCCATGGG | 4 |
| | (SEQ ID NO: 864) | |
| hs027562029 | GTACCTCATACAGCTGGCCCTGG | 4 |
| | (SEQ ID NO: 865) | |
| hs027562044 | GCCTGGGTGCACACACCCCTGGG | 4 |
| | (SEQ ID NO: 866) | |
| hs027561949 | TTCATTGCGGATCACCTCAGGGG | 5 |
| | (SEQ ID NO: 867) | |
| hs027561945 | TCTCAGACACAGGTTCATTGCGG | 5 |
| | (SEQ ID NO: 868) | |
| hs027561953 | AGTAGCCTGGATGGCCCCTGAGG | 5 |
| | (SEQ ID NO: 869) | |
| hs027561948 | GTTCATTGCGGATCACCTCAGGG | 5 |
| | (SEQ ID NO: 870) | |
| hs027561943 | ATGTCGACCTTCTCAGACACAGG | 5 |
| | (SEQ ID NO: 871) | |
| hs027561963 | GTCGTAGGTGATTAGCATGCTGG | 5 |
| | (SEQ ID NO: 872) | |
| hs027561946 | TGTCTGAGAAGGTCGACATCTGG | 5 |
| | (SEQ ID NO: 873) | |
| hs027561956 | CCTTTGCAGGGACAGTAGCCTGG | 5 |
| | (SEQ ID NO: 874) | |
| hs027561957 | GCTCTTGTCACTCAGCTCCTTGG | 5 |
| | (SEQ ID NO: 875) | |
| hs027561911 | AGGAATCTACGTCTTTGTAGGGG | 6 |
| | (SEQ ID NO: 876) | |
| hs027561918 | GCACCACGCCAAAGGACCTAGGG | 6 |
| | (SEQ ID NO: 877) | |
| hs027561900 | GAAACCATCTGGGCAACTGGAGG | 6 |
| | (SEQ ID NO: 878) | |
| hs027561903 | TCTGGGCAACTGGAGGGCACGGG | 6 |
| | (SEQ ID NO: 879) | |
| hs027561821 | TGTGGAGAGTACATCAGCTGAGG | 7 |
| | (SEQ ID NO: 880) | |
| hs027561815 | GACTTAAAGTAAGTCTCCTGGGG | 7 |
| | (SEQ ID NO: 881) | |
| hs027561824 | CCAGATGCAGCAGGATCTGTCGG | 7 |
| | (SEQ ID NO: 882) | |
| hs027561823 | AGGCAATGTCCAGATGCAGCAGG | 7 |
| | (SEQ ID NO: 883) | |
| hs027561818 | TTAAGTCCCAGGTGTGCTGTGGG | 7 |
| | (SEQ ID NO: 884) | |
| hs027561822 | TGATGTACTCTCCACACCCCAGG | 7 |
| | (SEQ ID NO: 885) | |
| hs027561826 | GCAGGATCTGTCGGAATGATGGG | 7 |
| | (SEQ ID NO: 886) | |
| hs027561762 | TCACCAGTTCCTCTTCTAGGCGG | 8 |
| | (SEQ ID NO: 887) | |
| hs027561770 | CTGTCTGCACCGCCTAGAAGAGG | 8 |
| | (SEQ ID NO: 888) | |
| hs027561771 | TTGAAAAGATTAAGTCAGAAGGG | 8 |
| | (SEQ ID NO: 889) | |
| hs027561756 | GTGAGTTCCCGTGCTTGGGGAGG | 8 |
| | (SEQ ID NO: 890) | |
| hs027561769 | GCACCGCCTAGAAGAGGAACTGG | 8 |
| | (SEQ ID NO: 891) | |
| hs027561707 | GAGTTCCAGCTGCAACATGAGGG | 9 |
| | (SEQ ID NO: 892) | |
| hs027561718 | GGAGCACTATGAAAGGAAGCTGG | 9 |
| | (SEQ ID NO: 893) | |
| hs027561656 | AAGGTGGGACATGAGTACTGTGG | 10 |
| | (SEQ ID NO: 894) | |
| hs027561669 | CCTCCTGCATGGAAACACAATGG | 10 |
| | (SEQ ID NO: 895) | |
| hs027561659 | TGTCACCCCATAGCAAAAGGTGG | 10 |
| | (SEQ ID NO: 896) | |
| hs027561658 | GTCACCCCATAGCAAAAGGTGGG | 10 |
| | (SEQ ID NO: 897) | |
| hs027561674 | CTGAAGCCACACCCTTCCCGGGG | 10 |
| | (SEQ ID NO: 898) | |
| hs027561663 | TGGAGAAGCTTATCAAGAAGAGG | 10 |
| | (SEQ ID NO: 899) | |
| hs027561655 | ATGTCCCACCTTTTGCTATGGGG | 10 |
| | (SEQ ID NO: 900) | |
| hs027561657 | CTATGGGGTGACAGCTTCTGTGG | 10 |
| | (SEQ ID NO: 901) | |
| hs027561660 | AGCTGTCACCCCATAGCAAAAGG | 10 |
| | (SEQ ID NO: 902) | |
| hs027561665 | GTTTCCATGCAGGAGGCCCCGGG | 10 |
| | (SEQ ID NO: 903) | |
| hs027561662 | CCATTGTGTTTCCATGCAGGAGG | 10 |
| | (SEQ ID NO: 904) | |
| hs027561667 | CATGCAGGAGGCCCCGGGAAGGG | 10 |
| | (SEQ ID NO: 905) | |
| hs027561675 | GCTGAAGCCACACCCTTCCCGGG | 10 |
| | (SEQ ID NO: 906) | |
| hs027561653 | CATGTCCCACCTTTTGCTATGGG | 10 |
| | (SEQ ID NO: 907) | |
| hs027561589 | GTGCCACCCCATGAACCTGGAGG | 11 |
| | (SEQ ID NO: 908) | |
| hs027561554 | GGTCATGATGAAGCCCACGGAGG | 11 |
| | (SEQ ID NO: 909) | |
| hs027561480 | CCTTCACCAGGCCCTACTGGAGG | 11 |
| | (SEQ ID NO: 910) | |
| hs027561505 | ACCAGCCCAGATTCACCTGGGGG | 11 |
| | (SEQ ID NO: 911) | |
| hs027561584 | TGGAGGACCAGGAAGCCCAGGGG | 11 |
| | (SEQ ID NO: 912) | |
| hs027561553 | GGAGGTCATGATGAAGCCCACGG | 11 |
| | (SEQ ID NO: 913) | |
| hs027561615 | TCACCAGGACGGAGTCGCCGTGG | 11 |
| | (SEQ ID NO: 914) | |
| hs027561630 | ACTAGATGCAGCCCTGAGTGGGG | 11 |
| | (SEQ ID NO: 915) | |
| hs027561451 | AAGTCAGGTAAAGTATGGGAAGG | 11 |
| | (SEQ ID NO: 916) | |
| hs027561545 | TCAGCAGCACTAGGGTCCCGGGG | 11 |
| | (SEQ ID NO: 917) | |
| hs027561617 | AGGGCCCCCCCTCACCAGGACGG | 11 |
| | (SEQ ID NO: 918) | |
| hs027561583 | GGAGGACCAGGAAGCCCAGGGGG | 11 |
| | (SEQ ID NO: 919) | |
| hs027561552 | AAGACATTTTGCGGAGCAGGAGG | 11 |
| | (SEQ ID NO: 920) | |
| hs027561493 | GGGAACCACCTCCTCCAGTAGGG | 11 |
| | (SEQ ID NO: 921) | |
| hs027561548 | ACCTGCTGTCAGCAGCACTAGGG | 11 |
| | (SEQ ID NO: 922) | |
| hs027561452 | CCCGAAGTCAGGTAAAGTATGGG | 11 |
| | (SEQ ID NO: 923) | |
| hs027561475 | TTCTGGGAACTGGAAGGGAAGGG | 11 |
| | (SEQ ID NO: 924) | |
| hs027561607 | TGGCAAGACCCGTCACCGCAAGG | 11 |
| | (SEQ ID NO: 925) | |
| hs027561555 | GTCATGATGAAGCCCACGGAGGG | 11 |
| | (SEQ ID NO: 926) | |
| hs027561450 | CTTTACCTGACTTCGGGTGACGG | 11 |
| | (SEQ ID NO: 927) | |
| hs027561507 | CCACCAGCCCAGATTCACCTGGG | 11 |
| | (SEQ ID NO: 928) | |
| hs027561613 | GACTCCGTCCTGGTGAGGGGGGG | 11 |
| | (SEQ ID NO: 929) | |
| hs027561620 | AGGAAGCCCCACCCCACTCAGGG | 11 |
| | (SEQ ID NO: 930) | |
| hs027561581 | GGTCCTCCAGGTTCATGGGGTGG | 11 |
| | (SEQ ID NO: 931) | |
| hs027561522 | GGCTCACCACCTCCGGCCCGGGG | 11 |
| | (SEQ ID NO: 932) | |
| hs027561490 | CCTCCAGTAGGGCCTGGTGAAGG | 11 |
| | (SEQ ID NO: 933) | |
| hs027561605 | TCACCGCAAGGCCAGCGCCAAGG | 11 |
| | (SEQ ID NO: 934) | |
| hs027561591 | GCTGTGCCACCCCATGAACCTGG | 11 |
| | (SEQ ID NO: 935) | |
| hs027561550 | GGGACGATGAAGACATTTTGCGG | 11 |
| | (SEQ ID NO: 936) | |
| hs027561602 | TGCGGTGACGGGTCTTGCCACGG | 11 |
| | (SEQ ID NO: 937) | |
| hs027561588 | GGCACAGCTGTACGAAGCCCAGG | 11 |
| | (SEQ ID NO: 938) | |
| hs027561460 | CCCAGCTGCACTGCTGTACAGGG | 11 |
| | (SEQ ID NO: 939) | |
| hs027561503 | ATTCACCTGGGGGAGCCAAAGGG | 11 |
| | (SEQ ID NO: 940) | |
| hs027561483 | CCAGGCCCTACTGGAGGAGGTGG | 11 |
| | (SEQ ID NO: 941) | |
| hs027561462 | TCAAGTGCTGGGACCCAGCCCGG | 11 |
| | (SEQ ID NO: 942) | |
| hs027561504 | GATTCACCTGGGGGAGCCAAAGG | 11 |
| | (SEQ ID NO: 943) | |
| hs027561539 | ACTAGGGTCCCGGGGCCGGGGGG | 11 |
| | (SEQ ID NO: 944) | |
| hs027561621 | GGGCTTCCTGGGTGTCCTAAGGG | 11 |
| | (SEQ ID NO: 945) | |
| hs027561491 | GGTTCCCCTTTGGCTCCCCCAGG | 11 |
| | (SEQ ID NO: 946) | |
| hs027561448 | TCCCATACTTTACCTGACTTCGG | 11 |
| | (SEQ ID NO: 947) | |
| hs027561568 | GTCCCCCTCCTAGGCCCCCTGGG | 11 |
| | (SEQ ID NO: 948) | |
| hs027561609 | CGGCGACTCCGTCCTGGTGAGGG | 11 |
| | (SEQ ID NO: 949) | |
| hs027561624 | TCAGGGCTGCATCTAGTTTAGGG | 11 |
| | (SEQ ID NO: 950) | |
| hs027561487 | GTAGGGCCTGGTGAAGGTGTGGG | 11 |
| | (SEQ ID NO: 951) | |
| hs027561403 | TAGAGCTGACATCAAGCCAGAGG | 12 |
| | (SEQ ID NO: 952) | |
| hs027561406 | ATCTCATCGGAAGAGGAGGAAGG | 12 |
| | (SEQ ID NO: 953) | |
| hs027561400 | GTAATGCCATCACTTACCTCTGG | 12 |
| | (SEQ ID NO: 954) | |
| hs027561401 | GATGGCATTACAAGAATTAATGG | 12 |
| | (SEQ ID NO: 955) | |
| hs027561379 | GCTAGTGAACCTTCCCCCAGTGG | 13 |
| | (SEQ ID NO: 956) | |
| hs027561373 | GCAGCACCAACACTGATGAGCGG | 13 |
| | (SEQ ID NO: 957) | |
| hs027561370 | CTGATGAGCGGCCAGATGAGCGG | 13 |
| | (SEQ ID NO: 958) | |
| hs027561383 | AGAGAATCCATCAGATGGGGAGG | 13 |
| | (SEQ ID NO: 959) | |
| hs027561358 | GGATGACCTCTGAAGGAGGTGGG | 13 |
| | (SEQ ID NO: 960) | |
| hs027561348 | ACCAGGAACTTCTCAGAGAGCGG | 13 |
| | (SEQ ID NO: 961) | |
| hs027561345 | TTCCTGGTGTGGAATGGGCAGGG | 13 |
| | (SEQ ID NO: 962) | |
| hs027561346 | CCAGGAACTTCTCAGAGAGCGGG | 13 |
| | (SEQ ID NO: 963) | |
| hs027561356 | CCAGGGATGACCTCTGAAGGAGG | 13 |
| | (SEQ ID NO: 964) | |
| hs027561365 | CCAGGGCTCAGAAATCCCACTGG | 13 |
| | (SEQ ID NO: 965) | |
| hs027561359 | TCTGAAGGAGGTGGGTCCAGTGG | 13 |
| | (SEQ ID NO: 966) | |
| hs027561343 | CTTCTCAGAGAGCGGGTGAGTGG | 13 |
| | (SEQ ID NO: 967) | |
| hs027561364 | ACTGGACCCACCTCCTTCAGAGG | 13 |
| | (SEQ ID NO: 968) | |
| hs027561357 | GGGATGACCTCTGAAGGAGGTGG | 13 |
| | (SEQ ID NO: 969) | |
| hs027561360 | CTGAAGGAGGTGGGTCCAGTGGG | 13 |
| | (SEQ ID NO: 970) | |
| hs027561367 | TCTGATGACATGTGCTCCCAGGG | 13 |
| | (SEQ ID NO: 971) | |
| hs027561369 | ATCTGGCCGCTCATCAGTGTTGG | 13 |
| | (SEQ ID NO: 972) | |
| hs027561341 | GAGAAGTTCCTGGTGTGGAATGG | 13 |
| | (SEQ ID NO: 973) | |
| hs027561378 | CCCAGTGGCACACCTGAAGTTGG | 13 |
| | (SEQ ID NO: 974) | |
| hs027561380 | GTGCCTTCCTCCCCATCTGATGG | 13 |
| | (SEQ ID NO: 975) | |
| hs027561374 | CCAACTTCAGGTGTGCCACTGGG | 13 |
| | (SEQ ID NO: 976) | |
| hs027561182 | CAGGGACTTAAACAGCACCCCGG | 14 |
| | (SEQ ID NO: 977) | |
| hs027561281 | ACATAATCAACAGAAAGATGGGG | 14 |
| | (SEQ ID NO: 978) | |
| hs027561199 | AGCCCTGTCTTAAGGCCCAGGGG | 14 |
| | (SEQ ID NO: 979) | |
| hs027561184 | TGGCTCAGGCTGAAGAACCGGGG | 14 |
| | (SEQ ID NO: 980) | |
| hs027561284 | CTATGTACAAGGAATACGAGTGG | 14 |
| | (SEQ ID NO: 981) | |
| hs027561175 | CAGGCACCAGGATAAAAGCAGGG | 14 |
| | (SEQ ID NO: 982) | |
| hs027561270 | TTATCAGGTGAATTGGTCAGGGG | 14 |
| | (SEQ ID NO: 983) | |
| hs027561169 | TATTTCAAGGTTTTTCACAGGGG | 14 |
| | (SEQ ID NO: 984) | |
| hs027561299 | TCTGAGTCCTCAGAATTGGGAGG | 14 |
| | (SEQ ID NO: 985) | |
| hs027561247 | ACAAGGCAATAGAAAAGCCAGGG | 14 |
| | (SEQ ID NO: 986) | |
| hs027561264 | GGCAGCTGTGGAAATGAATGAGG | 14 |
| | (SEQ ID NO: 987) | |
| hs027561198 | GCCCTGTCTTAAGGCCCAGGGGG | 14 |
| | (SEQ ID NO: 988) | |
| hs027561296 | CCAACAGCGTTGATGCCTTGCGG | 14 |
| | (SEQ ID NO: 989) | |
| hs027561286 | AATACGAGTGGCTTTCATGGAGG | 14 |
| | (SEQ ID NO: 990) | |
| hs027561289 | GAGTGGCTTTCATGGAGGGAGGG | 14 |
| | (SEQ ID NO: 991) | |
| hs027561242 | CTTTATCCCCAAATAATAGGGGG | 14 |
| | (SEQ ID NO: 992) | |
| hs027561252 | GCTGAATATAAGGAATGGGGTGG | 14 |
| | (SEQ ID NO: 993) | |
| hs027561257 | CTTATATTCAGCAAGCAACAAGG | 14 |
| | (SEQ ID NO: 994) | |
| hs027561300 | CTGAGTCCTCAGAATTGGGAGGG | 14 |
| | (SEQ ID NO: 995) | |
| hs027561272 | AATTCACCTGATAAACTCTAGGG | 14 |
| | (SEQ ID NO: 996) | |
| hs027561222 | ACAGAAAAGTGCAGTCTAAGTGG | 14 |
| | (SEQ ID NO: 997) | |
| hs027561168 | ATATTTCAAGGTTTTTCACAGGG | 14 |
| | (SEQ ID NO: 998) | |
| hs027561170 | TGAAATATAACACTCCATGCAGG | 14 |
| | (SEQ ID NO: 999) | |
| hs027561277 | CAGCCGTAAGTCAGGCTCGAGGG | 14 |
| | (SEQ ID NO: 1000) | |
| hs027561243 | TCTTTATCCCCAAATAATAGGGG | 14 |
| | (SEQ ID NO: 1001) | |
| hs027561219 | TTACACATTTTGCACTTGGGAGG | 14 |
| | (SEQ ID NO: 1002) | |
| hs027561287 | ATACGAGTGGCTTTCATGGAGGG | 14 |
| | (SEQ ID NO: 1003) | |
| hs027561171 | TTTTTCACAGGGGTTACAGTAGG | 14 |
| | (SEQ ID NO: 1004) | |
| hs027561253 | CTGAATATAAGGAATGGGGTGGG | 14 |
| | (SEQ ID NO: 1005) | |
| hs027561274 | TCTCCCTCGAGCCTGACTTACGG | 14 |
| | (SEQ ID NO: 1006) | |
| hs027561200 | AAGCCCTGTCTTAAGGCCCAGGG | 14 |
| | (SEQ ID NO: 1007) | |
| hs027561240 | TATCCCCAAATAATAGGGGGTGG | 14 |
| | (SEQ ID NO: 1008) | |
| hs027561188 | CAGTCTCCTAACCTCCCCCTGGG | 14 |
| | (SEQ ID NO: 1009) | |
| hs027561174 | TCAGGCACCAGGATAAAAGCAGG | 14 |
| | (SEQ ID NO: 1010) | |
| hs027561254 | TGAATATAAGGAATGGGGTGGGG | 14 |
| | (SEQ ID NO: 1011) | |
| hs027561209 | CCCCTCTGAGGTTTCTCAGGTGG | 14 |
| | (SEQ ID NO: 1012) | |
| hs027561276 | CTCGAGCCTGACTTACGGCTGGG | 14 |
| | (SEQ ID NO: 1013) | |
| hs027561294 | GAGGGAAGCTGGGGGCCGCAAGG | 14 |
| | (SEQ ID NO: 1014) | |
| hs027561250 | GCTTGCTGAATATAAGGAATGGG | 14 |
| | (SEQ ID NO: 1015) | |
| hs027561260 | TGCCCTTAGCCACAGCTCTACGG | 14 |
| | (SEQ ID NO: 1016) | |
| hs027561187 | TCAGTCTCCTAACCTCCCCCTGG | 14 |
| | (SEQ ID NO: 1017) | |
| hs027561165 | CATGCAGGCCCAGCTGTTGAGGG | 14 |
| | (SEQ ID NO: 1018) | |
| hs027561194 | TGGGCCTTAAGACAGGGCTTGGG | 14 |
| | (SEQ ID NO: 1019) | |
| hs027561164 | GCATGGAGTGTTATATTTCAAGG | 14 |
| | (SEQ ID NO: 1020) | |
| hs027561275 | CCTCGAGCCTGACTTACGGCTGG | 14 |
| | (SEQ ID NO: 1021) | |
| hs027561197 | GCTTGGGCAGAGAAGATAAATGG | 14 |
| | (SEQ ID NO: 1022) | |
| hs027561244 | TTCTTTATCCCCAAATAATAGGG | 14 |
| | (SEQ ID NO: 1023) | |

TABLE 9
Map3K13 (LZK) exonic target sites
SpCas9

| grID | sequence | exon |
|---|---|---|
| hs088398521 | GGGAGCCTAGGATCCCCGACAGG | 1 |
| | (SEQ ID NO: 1024) | |
| hs088398501 | AGGAAGTTACGCACGGGAGGCGG | 1 |
| | (SEQ ID NO: 1025) | |
| hs088398507 | AGGCGGTACCTGGTTGTGGAGGG | 1 |
| | (SEQ ID NO: 1026) | |
| hs088398520 | CAGACGCTCCGCCCCACCCATGG | 1 |
| | (SEQ ID NO: 1027) | |
| hs088398506 | GAGGCGGTACCTGGTTGTGGAGG | 1 |
| | (SEQ ID NO: 1028) | |
| hs088398508 | GGTTGTGGAGGGTGACGCCATGG | 1 |
| | (SEQ ID NO: 1029) | |
| hs088398518 | CGGAGCGTCTGGGATGCGCTGGG | 1 |
| | (SEQ ID NO: 1030) | |
| hs088398513 | GGGTGACGCCATGGGTGGGGCGG | 1 |
| | (SEQ ID NO: 1031) | |
| hs088398512 | GGAGGGTGACGCCATGGGTGGGG | 1 |
| | (SEQ ID NO: 1032) | |
| hs088398712 | ACTTCATTTAAAGAAACCCACGG | 2 |
| | (SEQ ID NO: 1033) | |
| hs088398720 | AAATCCTAGCACTCTGGGAGAGG | 2 |
| | (SEQ ID NO: 1034) | |
| hs088398716 | ACATGAGTCAATATGTATTGTGG | 2 |
| | (SEQ ID NO: 1035) | |
| hs088403650 | TCAGGGGAACAGCAACACGGTGG | 3 |
| | (SEQ ID NO: 1036) | |
| hs088403645 | ACACGATGAATCAGAGACGGCGG | 3 |
| | (SEQ ID NO: 1037) | |
| hs088403619 | AAGATGAGCTCACAGCTATGGGG | 3 |
| | (SEQ ID NO: 1038) | |
| hs088403598 | AAAACCTGAGAAAAACAGGAGGG | 3 |
| | (SEQ ID NO: 1039) | |
| hs088403644 | GGAACACGATGAATCAGAGACGG | 3 |
| | (SEQ ID NO: 1040) | |
| hs088403599 | CAAAACCTGAGAAAAACAGGAGG | 3 |
| | (SEQ ID NO: 1041) | |
| hs088403649 | GTCTCAGGGGAACAGCAACACGG | 3 |
| | (SEQ ID NO: 1042) | |
| hs088403626 | CGAGCAGCTTGGGAGAAGGGTGG | 3 |
| | (SEQ ID NO: 1043) | |
| hs088403601 | CAGAGTTCTGACTTAAGAGAGGG | 3 |
| | (SEQ ID NO: 1044) | |
| hs088403625 | TCGAGGACCAGCAGGAAAAGGGG | 3 |
| | (SEQ ID NO: 1045) | |
| hs088403667 | GATTACAAATTGCAGCAGCAAGG | 3 |
| | (SEQ ID NO: 1046) | |
| hs088403608 | TGGTTATACTCATGGCACGATGG | 3 |
| | (SEQ ID NO: 1047) | |
| hs088403602 | ACAGAGTTCTGACTTAAGAGAGG | 3 |
| | (SEQ ID NO: 1048) | |
| hs088403634 | TCAACACTGTTGTGGTGACGGGG | 3 |
| | (SEQ ID NO: 1049) | |
| hs088403633 | AGTGTTGACGAGCGTAAGTGAGG | 3 |
| | (SEQ ID NO: 1050) | |
| hs088403652 | GTGGACGGAGAGAGCACAAGCGG | 3 |
| | (SEQ ID NO: 1051) | |
| hs088403639 | TACGCTCGTCAACACTGTTGTGG | 3 |
| | (SEQ ID NO: 1052) | |
| hs088403638 | GAGCGTAAGTGAGGATTCCAGGG | 3 |
| | (SEQ ID NO: 1053) | |
| hs088403641 | GAACAGCGTTCTTCAGCTAAGGG | 3 |
| | (SEQ ID NO: 1054) | |
| hs088403614 | CTTTCACTGAAGGGTAAGTGTGG | 3 |
| | (SEQ ID NO: 1055) | |
| hs088403651 | GGGAACAGCAACACGGTGGACGG | 3 |
| | (SEQ ID NO: 1056) | |
| hs088403655 | CAGTTCAGCAGGTCAGGCAGTGG | 3 |
| | (SEQ ID NO: 1057) | |
| hs088403610 | GCTCAGGTGCTCCTGAAAGTTGG | 3 |
| | (SEQ ID NO: 1058) | |
| hs088403622 | CAAGCTGCTCGAGGACCAGCAGG | 3 |
| | (SEQ ID NO: 1059) | |
| hs088403642 | GCTGTTCTCAAACTGGTCCCTGG | 3 |
| | (SEQ ID NO: 1060) | |
| hs088403664 | GGCCTGTATGGAATATCATTGGG | 3 |
| | (SEQ ID NO: 1061) | |
| hs088403613 | AGTGAAAGCAAAACCTTCAATGG | 3 |
| | (SEQ ID NO: 1062) | |
| hs088403623 | GCTCGAGGACCAGCAGGAAAAGG | 3 |
| | (SEQ ID NO: 1063) | |
| hs088403663 | AGGCCTGTATGGAATATCATTGG | 3 |
| | (SEQ ID NO: 1064) | |
| hs088403632 | GTACCATCCCCTTTTCCTGCTGG | 3 |
| | (SEQ ID NO: 1065) | |
| hs088403659 | GGCAGTGGTGGGTTTCTTGAAGG | 3 |
| | (SEQ ID NO: 1066) | |
| hs088404068 | CTTCTTGGGCAAGTTCCGGGCGG | 4 |
| | (SEQ ID NO: 1067) | |
| hs088404063 | GGGTAGTGGAGCCCAAGGAGCGG | 4 |
| | (SEQ ID NO: 1068) | |
| hs088404074 | AGTGAGAGAACAGAATGAGACGG | 4 |
| | (SEQ ID NO: 1069) | |
| hs088404077 | TAACATCATCGCATTCAAGTAGG | 4 |
| | (SEQ ID NO: 1070) | |
| hs088404054 | GCTTGTGTGCCTAGATACTTGGG | 4 |
| | (SEQ ID NO: 1071) | |
| hs088404073 | TGATGGCCACCTCTTCCGCCCGG | 4 |
| | (SEQ ID NO: 1072) | |
| hs088404060 | GAGCTGCAGTGGCTGGGTAGTGG | 4 |
| | (SEQ ID NO: 1073) | |
| hs088404075 | CTGTTCTCTCACTTTCTTGATGG | 4 |
| | (SEQ ID NO: 1074) | |
| hs088404379 | TGCCCATGGACAACTCTACGAGG | 5 |
| | (SEQ ID NO: 1075) | |
| hs088404376 | CATGATAATACAATAACATGGGG | 5 |
| | (SEQ ID NO: 1076) | |
| hs088404382 | GACCTCGTAGAGTTGTCCATGGG | 5 |
| | (SEQ ID NO: 1077) | |
| hs088404386 | TGGTCCACAGGAATTGCAAGTGG | 5 |
| | (SEQ ID NO: 1078) | |
| hs088404377 | CCATGATAATACAATAACATGGG | 5 |
| | (SEQ ID NO: 1079) | |
| hs088404378 | TCCATGATAATACAATAACATGG | 5 |
| | (SEQ ID NO: 1080) | |
| hs088404385 | TTGCTAGTAGACTGGTCCACAGG | 5 |
| | (SEQ ID NO: 1081) | |
| hs088404388 | CATTCCACTTGCAATTCCTGTGG | 5 |
| | (SEQ ID NO: 1082) | |
| hs088404381 | ACGAGGTCTTACGAGCTGGCAGG | 5 |
| | (SEQ ID NO: 1083) | |
| hs088404390 | GATCACGATGAATAATTTTATGG | 5 |
| | (SEQ ID NO: 1084) | |
| hs088404569 | TTTAGTGACCCACACAGATGCGG | 6 |
| | (SEQ ID NO: 1085) | |
| hs088404573 | AAATTTTTACCGCATCTGTGTGG | 6 |
| | (SEQ ID NO: 1086) | |
| hs088404579 | GGTCGCATGGATGGCGCCAGAGG | 6 |
| | (SEQ ID NO: 1087) | |
| hs088404576 | CATTTGCTGGCACGGTCGCATGG | 6 |
| | (SEQ ID NO: 1088) | |
| hs088404580 | GGATGGCGCCAGAGGTGATACGG | 6 |
| | (SEQ ID NO: 1089) | |
| hs088404581 | GGTTCATTCCGTATCACCTCTGG | 6 |
| | (SEQ ID NO: 1090) | |
| hs088404659 | AAATCCTTATGAAACAGACGTGG | 7 |
| | (SEQ ID NO: 1091) | |
| hs088404655 | AAGTGGAAGGAACTGGAAGGTGG | 7 |
| | (SEQ ID NO: 1092) | |
| hs088404649 | AAGAATCTACATCTTTGTAAGGG | 7 |
| | (SEQ ID NO: 1093) | |
| hs088404658 | AATCCATCAGGGCAAGTGGAAGG | 7 |
| | (SEQ ID NO: 1094) | |
| hs088404643 | AAAAGACCTACAGTAGCGCCGGG | 7 |
| | (SEQ ID NO: 1095) | |
| hs088404648 | GATTCTTCAGCCATTATCTGGGG | 7 |
| | (SEQ ID NO: 1096) | |
| hs088404639 | CTACTGTAGGTCTTTTGGAGTGG | 7 |
| | (SEQ ID NO: 1097) | |
| hs088404644 | CAAAAGACCTACAGTAGCGCCGG | 7 |
| | (SEQ ID NO: 1098) | |
| hs088404657 | TCAGGGCAAGTGGAAGGAACTGG | 7 |
| | (SEQ ID NO: 1099) | |
| hs088404662 | ATAAGGATTTTGAATCCATCAGG | 7 |
| | (SEQ ID NO: 1100) | |
| hs088404653 | GTTCCTTCCACTTGCCCTGATGG | 7 |
| | (SEQ ID NO: 1101) | |
| hs088404652 | GCTTCCAACACCCCAGATAATGG | 7 |
| | (SEQ ID NO: 1102) | |
| hs088404684 | GTGTCTGCCGAAAAGAAGGTCGG | 8 |
| | (SEQ ID NO: 1103) | |
| hs088404686 | GGTGGCAAGTACATCTGCAGAGG | 8 |
| | (SEQ ID NO: 1104) | |
| hs088404691 | CTTGAAGTAAGTTTCTTGTGGGG | 8 |
| | (SEQ ID NO: 1105) | |
| hs088404690 | GAAGTAAGTTTCTTGTGGGGTGG | 8 |
| | (SEQ ID NO: 1106) | |
| hs088404685 | ATGAGTGTCTGCCGAAAAGAAGG | 8 |
| | (SEQ ID NO: 1107) | |
| hs088404687 | AGAAACTTACTTCAAGTCTCAGG | 8 |
| | (SEQ ID NO: 1108) | |
| hs088404683 | CGAAAAGAAGGTCGGTTTCGAGG | 8 |
| | (SEQ ID NO: 1109) | |
| hs088404682 | CTCGAAACCGACCTTCTTTTCGG | 8 |
| | (SEQ ID NO: 1110) | |
| hs088405101 | GTGAAGGAACTTGTATACACCGG | 9 |
| | (SEQ ID NO: 1111) | |
| hs088405100 | TTTGAGAAGATCAAAAGTGAAGG | 9 |
| | (SEQ ID NO: 1112) | |
| hs088405104 | GAAGGCGCAGAGAAGAGCTCAGG | 9 |
| | (SEQ ID NO: 1113) | |
| hs088405103 | GAATCAGTTCTTCATCTAACCGG | 9 |
| | (SEQ ID NO: 1114) | |
| hs088405096 | TTGTCCTTACCCAGGCTGAATGG | 9 |
| | (SEQ ID NO: 1115) | |
| hs088405099 | CTTCTTCTCTCCATTCAGCCTGG | 9 |
| | (SEQ ID NO: 1116) | |
| hs088405182 | TGCCTGTCAGAGAAAACACAGGG | 10 |
| | (SEQ ID NO: 1117) | |
| hs088405184 | ATATTCGTGAACACTATGAGCGG | 10 |
| | (SEQ ID NO: 1118) | |
| hs088405185 | ACTATGAGCGGAAGCTTGAGCGG | 10 |
| | (SEQ ID NO: 1119) | |
| hs088405188 | TCATGCTGCAGCTAGAAATGCGG | 10 |
| | (SEQ ID NO: 1120) | |
| hs088405187 | GCGGGCGAATAATTTATACATGG | 10 |
| | (SEQ ID NO: 1121) | |
| hs088405186 | CTATGAGCGGAAGCTTGAGCGGG | 10 |
| | (SEQ ID NO: 1122) | |
| hs088405191 | CATTTCTAGCTGCAGCATGATGG | 10 |
| | (SEQ ID NO: 1123) | |
| hs088405181 | TTTCTCTGACAGGCATGCGCTGG | 10 |
| | (SEQ ID NO: 1124) | |
| hs088405272 | GATGGATGATAGGACGAACAGGG | 11 |
| | (SEQ ID NO: 1125) | |
| hs088405265 | CTGCAGGCGTGAGCAAGCAGTGG | 11 |
| | (SEQ ID NO: 1126) | |
| hs088405271 | TATCATCCATCCCAATGCCATGG | 11 |
| | (SEQ ID NO: 1127) | |
| hs088405277 | GTTTCTCCATGGCATTGGGATGG | 11 |
| | (SEQ ID NO: 1128) | |
| hs088405276 | GAGAAACTCATGAAAAGGAAAGG | 11 |
| | (SEQ ID NO: 1129) | |
| hs088405283 | AATCTGGGATGCAGACCAAACGG | 11 |
| | (SEQ ID NO: 1130) | |
| hs088405274 | CCATGGAGAAACTCATGAAAAGG | 11 |
| | (SEQ ID NO: 1131) | |
| hs088405273 | GGATGGATGATAGGACGAACAGG | 11 |
| | (SEQ ID NO: 1132) | |
| hs088405280 | CCTTTTCATGAGTTTCTCCATGG | 11 |
| | (SEQ ID NO: 1133) | |
| hs088405266 | TGCTCACGCCTGCAGAATAAAGG | 11 |
| | (SEQ ID NO: 1134) | |
| hs088405558 | CAGACTTGTTGAGATCAGAAGGG | 12 |
| | (SEQ ID NO: 1135) | |
| hs088405679 | ACGGAAGAAAATGAATTCAGCGG | 12 |
| | (SEQ ID NO: 1136) | |
| hs088405620 | CAACCTGCGCCAACAACCTGAGG | 12 |
| | (SEQ ID NO: 1137) | |
| hs088405611 | CATCCCAGACTCAATATGCACGG | 12 |
| | (SEQ ID NO: 1138) | |
| hs088405643 | AGAAGTTCTGAGCCTGACAAGGG | 12 |
| | (SEQ ID NO: 1139) | |
| hs088405572 | TTTGGGACTTCCGGACAAAGGGG | 12 |
| | (SEQ ID NO: 1140) | |
| hs088405683 | TGTAGGTCTGAGTCATCCCTCGG | 12 |
| | (SEQ ID NO: 1141) | |
| hs088405585 | TGCCTCTGCTATTCCCTCGGCGG | 12 |
| | (SEQ ID NO: 1142) | |
| hs088405664 | ATACCCTCTGCTGAGCCAGTGGG | 12 |
| | (SEQ ID NO: 1143) | |
| hs088405582 | CACCGCCGAGGGAATAGCAGAGG | 12 |
| | (SEQ ID NO: 1144) | |
| hs088405687 | CAGCGCTACCTCGAAAAACAAGG | 12 |
| | (SEQ ID NO: 1145) | |
| hs088405618 | TGTCCGTGCATATTGAGTCTGGG | 12 |
| | (SEQ ID NO: 1146) | |
| hs088405657 | AGTGCAGGCCAGAACAGTATGGG | 12 |
| | (SEQ ID NO: 1147) | |
| hs088405630 | CATGCTCAGAGACAGCTGCCCGG | 12 |
| | (SEQ ID NO: 1148) | |
| hs088405579 | AGCAAACCACGCCACCGCCGAGG | 12 |
| | (SEQ ID NO: 1149) | |
| hs088405632 | CTCTGAGCATGGTTGCTGAGTGG | 12 |
| | (SEQ ID NO: 1150) | |
| hs088405603 | AATTATGGTGATGAAGAGAAGGG | 12 |
| | (SEQ ID NO: 1151) | |
| hs088405688 | TGGAGGGGTGCCGAGATGAGAGG | 12 |
| | (SEQ ID NO: 1152) | |
| hs088405628 | GTTGCTGAGTGGGCTCCGCAGGG | 12 |
| | (SEQ ID NO: 1153) | |
| hs088405580 | GCAAACCACGCCACCGCCGAGGG | 12 |
| | (SEQ ID NO: 1154) | |
| hs088405686 | TGCCGAGATGAGAGGTGCCGAGG | 12 |
| | (SEQ ID NO: 1155) | |
| hs088405592 | GGGATGGGGTGAATTTTCCTGGG | 12 |
| | (SEQ ID NO: 1156) | |
| hs088405685 | GCCGAGATGAGAGGTGCCGAGGG | 12 |
| | (SEQ ID NO: 1157) | |
| hs088405557 | CCAGACTTGTTGAGATCAGAAGG | 12 |
| | (SEQ ID NO: 1158) | |
| hs088405583 | CTATTCCCTCGGCGGTGGCGTGG | 12 |
| | (SEQ ID NO: 1159) | |
| hs088405636 | GATGAGGTCAGGGCTCGAGCCGG | 12 |
| | (SEQ ID NO: 1160) | |
| hs088405625 | CTGCTGGGCCGAAATACCTCAGG | 12 |
| | (SEQ ID NO: 1161) | |
| hs088405644 | TCTCCAGCAGTCTGCAGCCATGG | 12 |
| | (SEQ ID NO: 1162) | |
| hs088405586 | GGCTGCCTCTGCTATTCCCTCGG | 12 |
| | (SEQ ID NO: 1163) | |
| hs088405588 | AGATTGCGGCAAAGTCACTATGG | 12 |
| | (SEQ ID NO: 1164) | |
| hs088405613 | GGGATGGTGACTCTGGGACATGG | 12 |
| | (SEQ ID NO: 1165) | |
| hs088405599 | ATTGGGATTGAGCTTGGTGCAGG | 12 |
| | (SEQ ID NO: 1166) | |
| hs088405634 | TGACCTCATCTCCACAGCCATGG | 12 |
| | (SEQ ID NO: 1167) | |
| hs088405561 | GATCTCAACAAGTCTGGCCTGGG | 12 |
| | (SEQ ID NO: 1168) | |
| hs088405691 | TTTTTCGAGGTAGCGCTGGAGGG | 12 |
| | (SEQ ID NO: 1169) | |
| hs088405624 | GCCGAAATACCTCAGGTTGTTGG | 12 |
| | (SEQ ID NO: 1170) | |
| hs088405573 | TTTTGGGACTTCCGGACAAAGGG | 12 |
| | (SEQ ID NO: 1171) | |
| hs088405622 | ATACCTCAGGTTGTTGGCGCAGG | 12 |
| | (SEQ ID NO: 1172) | |
| hs088405659 | TCTGGCCTGCACTGAAGGCAGGG | 12 |
| | (SEQ ID NO: 1173) | |
| hs088405876 | TTGAATTTCCACGAAGACAGAGG | 13 |
| | (SEQ ID NO: 1174) | |
| hs088405869 | TGGAGATGACTCCTCAGAAGAGG | 13 |
| | (SEQ ID NO: 1175) | |
| hs088405870 | TCTCCACTCTGAGGAGAACAAGG | 13 |
| | (SEQ ID NO: 1176) | |
| hs088405875 | TTCCCCTTCTTCCTCTTCTGAGG | 13 |
| | (SEQ ID NO: 1177) | |
| hs088406081 | GAGACATCTGAGTCTTCACACGG | 14 |
| | (SEQ ID NO: 1178) | |
| hs088406064 | CGTCTAGCTTCTCTGCCAAGCGG | 14 |
| | (SEQ ID NO: 1179) | |
| hs088406052 | CAGCTGCTGATACAGCGATGGGG | 14 |
| | (SEQ ID NO: 1180) | |
| hs088406061 | ACTCATCAGGACTGTTTGAGTGG | 14 |
| | (SEQ ID NO: 1181) | |
| hs088406051 | GCCTAGAACCAAGAAGAACCAGG | 14 |
| | (SEQ ID NO: 1182) | |
| hs088406066 | CATTGACATATCCTCACACTCGG | 14 |
| | (SEQ ID NO: 1183) | |
| hs088406078 | CTGAGTCTTCACACGGCGCACGG | 14 |
| | (SEQ ID NO: 1184) | |
| hs088406058 | TCTGTGTCTGATGGAGAAGAGGG | 14 |
| | (SEQ ID NO: 1185) | |
| hs088406070 | ACATATCCTCACACTCGGATGGG | 14 |
| | (SEQ ID NO: 1186) | |
| hs088406062 | TGATAAACTTGAAGACCGCTTGG | 14 |
| | (SEQ ID NO: 1187) | |
| hs088406084 | GGAGGAACGTGGCTATGAGGTGG | 14 |
| | (SEQ ID NO: 1188) | |
| hs088406073 | GAGTGTGAGGATATGTCAATGGG | 14 |
| | (SEQ ID NO: 1189) | |
| hs088406069 | GACATATCCTCACACTCGGATGG | 14 |
| | (SEQ ID NO: 1190) | |
| hs088406054 | GGCAGCTGCTGATACAGCGATGG | 14 |
| | (SEQ ID NO: 1191) | |
| hs088406074 | CGAGTGTGAGGATATGTCAATGG | 14 |
| | (SEQ ID NO: 1192) | |
| hs088406053 | GCAGCTGCTGATACAGCGATGGG | 14 |
| | (SEQ ID NO: 1193) | |
| hs088406317 | ATGCAATCAGTAAAACCCAGAGG | 15 |
| | (SEQ ID NO: 1194) | |
| hs088406249 | GATCACTCAGTTCAGCTACGAGG | 15 |
| | (SEQ ID NO: 1195) | |
| hs088406380 | ATAAAGGTGGGAGAACAAAGGGG | 15 |
| | (SEQ ID NO: 1196) | |
| hs088406514 | AGGATGCCAATGTTCCACAGTGG | 15 |
| | (SEQ ID NO: 1197) | |
| hs088406420 | AAAAACTGATCAAAATTACGAGG | 15 |
| | (SEQ ID NO: 1198) | |
| hs088406245 | GAGAGGAAGAACATCTACAGTGG | 15 |
| | (SEQ ID NO: 1199) | |
| hs088406591 | AAATGCTCCAGGTTCCACAAGGG | 15 |
| | (SEQ ID NO: 1200) | |
| hs088406375 | ACGAATCCATGAGCCCTGTGCGG | 15 |
| | (SEQ ID NO: 1201) | |
| hs088406360 | TCCACTATTTGACATCCTGGGGG | 15 |
| | (SEQ ID NO: 1202) | |
| hs088406675 | GACAGTACAGGAGACCAGAGGGG | 15 |
| | (SEQ ID NO: 1203) | |
| hs088406254 | AACCTGGCTCCAGAGAGGGGTGG | 15 |
| | (SEQ ID NO: 1204) | |
| hs088406677 | CTGACAGTACAGGAGACCAGAGG | 15 |
| | (SEQ ID NO: 1205) | |
| hs088406583 | GACTGAACTAGGCTAAGCAGAGG | 15 |
| | (SEQ ID NO: 1206) | |
| hs088406417 | TCAAAATTACGAGGTATCAGGGG | 15 |
| | (SEQ ID NO: 1207) | |
| hs088406235 | CCTGTAGGAACAAAGGTTGGGGG | 15 |
| | (SEQ ID NO: 1208) | |
| hs088406341 | CACAGCTAAGATCAATACCAGGG | 15 |
| | (SEQ ID NO: 1209) | |
| hs088406409 | GTGGCAACAGAGAAACATGAGGG | 15 |
| | (SEQ ID NO: 1210) | |
| hs088406653 | CTGAAGGTGAAGAGCCACTGGGG | 15 |
| | (SEQ ID NO: 1211) | |
| hs088406690 | CATTGCCGTTCAATACAGAGAGG | 15 |
| | (SEQ ID NO: 1212) | |
| hs088406448 | CAACACTCTTCAGATGACTGTGG | 15 |
| | (SEQ ID NO: 1213) | |
| hs088406648 | TCTTGCCCATATCACCCCAGTGG | 15 |
| | (SEQ ID NO: 1214) | |
| hs088406408 | AGTGGCAACAGAGAAACATGAGG | 15 |
| | (SEQ ID NO: 1215) | |
| hs088406329 | AGAAGGTGTGATTTATGATGTGG | 15 |
| | (SEQ ID NO: 1216) | |
| hs088406689 | ATTGCCGTTCAATACAGAGAGGG | 15 |
| | (SEQ ID NO: 1217) | |
| hs088406475 | ACAGGTCTAAACTTAAGTGGTGG | 15 |
| | (SEQ ID NO: 1218) | |
| hs088406247 | TGGTGTTAGGAAAACGAACGTGG | 15 |
| | (SEQ ID NO: 1219) | |
| hs088406565 | AGTCTAAGAAGCAAGTAACTGGG | 15 |
| | (SEQ ID NO: 1220) | |
| hs088406520 | CAGTTCCAGGAAGTTCAAGTGGG | 15 |
| | (SEQ ID NO: 1221) | |
| hs088406383 | TCTCCTAGTGGAGAGTAGCAGGG | 15 |
| | (SEQ ID NO: 1222) | |
| hs088406226 | AGTATGAAAGATCAACACGTTGG | 15 |
| | (SEQ ID NO: 1223) | |
| hs088406581 | CTGCAAAAGCCTATTCAAGTCGG | 15 |
| | (SEQ ID NO: 1224) | |
| hs088406229 | GCCTGCTGACCACTTCCTGTAGG | 15 |
| | (SEQ ID NO: 1225) | |
| hs088406172 | TATTGCAGCTCATTTCTAGGGGG | 15 |
| | (SEQ ID NO: 1226) | |
| hs088406180 | CATTTCAATCAAGAATGGCAGGG | 15 |
| | (SEQ ID NO: 1227) | |
| hs088406307 | AGAGTCCAGTGAATCTAGAGAGG | 15 |
| | (SEQ ID NO: 1228) | |
| hs088406343 | CTGTGTACAACAATCATCTGAGG | 15 |
| | (SEQ ID NO: 1229) | |
| hs088406330 | TGATTTATGATGTGGCACAATGG | 15 |
| | (SEQ ID NO: 1230) | |
| hs088406223 | TCAAGGTAGCACATTTGCCAAGG | 15 |
| | (SEQ ID NO: 1231) | |
| hs088406664 | ACTGTAATAAAGCGTCAAAGAGG | 15 |
| | (SEQ ID NO: 1232) | |
| hs088406163 | ACAAAATGCTGGGAGAGAGTGGG | 15 |
| | (SEQ ID NO: 1233) | |
| hs088406288 | GGGCTCTGAAAAGCATTCCAAGG | 15 |
| | (SEQ ID NO: 1234) | |
| hs088406663 | CTGTAATAAAGCGTCAAAGAGGG | 15 |
| | (SEQ ID NO: 1235) | |
| hs088406210 | CTTGCACCCAATGTCTTCAAGGG | 15 |
| | (SEQ ID NO: 1236) | |
| hs088406605 | GTCATGTGGCCAGGGTACGATGG | 15 |
| | (SEQ ID NO: 1237) | |
| hs088406658 | ACTTCCCAGACTCTGGAAGTGGG | 15 |
| | (SEQ ID NO: 1238) | |
| hs088406615 | TGCCGGGAGCTTCCTTACAGTGG | 15 |
| | (SEQ ID NO: 1239) | |
| hs088406236 | CTGTAGGAACAAAGGTTGGGGGG | 15 |
| | (SEQ ID NO: 1240) | |
| hs088406668 | TTCATTCGCTCCAAGTCGATAGG | 15 |
| | (SEQ ID NO: 1241) | |
| hs088406456 | CATGGACATTCATCCCAAGATGG | 15 |
| | (SEQ ID NO: 1242) | |
| hs088406282 | TTTACATTGCCAAAAGCATGAGG | 15 |
| | (SEQ ID NO: 1243) | |
| hs088406444 | AGATTTTCCCGGTTATCACAAGG | 15 |
| | (SEQ ID NO: 1244) | |
| hs088406389 | TCTCCCTGCTACTCTCCACTAGG | 15 |
| | (SEQ ID NO: 1245) | |
| hs088406189 | CCCGGCCCCACAAATTCCCAAGG | 15 |
| | (SEQ ID NO: 1246) | |
| hs088406230 | ACCACTTCCTGTAGGAACAAAGG | 15 |
| | (SEQ ID NO: 1247) | |
| hs088406466 | TAAAAGGAACTGTTATCCAGAGG | 15 |
| | (SEQ ID NO: 1248) | |
| hs088406353 | TGATCATGAAAGCTCCTGGGTGG | 15 |
| | (SEQ ID NO: 1249) | |
| hs088406579 | TCTAATGTGCCAGTCTGTGAGGG | 15 |
| | (SEQ ID NO: 1250 | |
| hs088406142 | CCGATTCTTCAAACTGCATGGGG | 15 |
| | (SEQ ID NO: 1251) | |
| hs088406526 | GCACACTCTACTGCAGAGCAGGG | 15 |
| | (SEQ ID NO: 1252) | |
| hs088406354 | GATCATGAAAGCTCCTGGGTGGG | 15 |
| | (SEQ ID NO: 1253) | |
| hs088406609 | AGAGCCCTAAGCAGATGGCAAGG | 15 |
| | (SEQ ID NO: 1254) | |
| hs088406185 | AACATCCTTGGGAATTTGTGGGG | 15 |
| | (SEQ ID NO: 1255) | |
| hs088406314 | ATCAGTAAAACCCAGAGGAGCGG | 15 |
| | (SEQ ID NO: 1256) | |
| hs088406342 | ACACAGCTAAGATCAATACCAGG | 15 |
| | (SEQ ID NO: 1257) | |
| hs088406590 | GAGCATTTCTCAGGGACAGGTGG | 15 |
| | (SEQ ID NO: 1258) | |
| hs088406684 | GGCTCCCTCTCTGTATTGAACGG | 15 |
| | (SEQ ID NO: 1259) | |
| hs088406453 | CTTGAAATCACAGTCTTGTGAGG | 15 |
| | (SEQ ID NO: 1260) | |
| hs088406680 | GAAAACAACACTGGGAGCTGGGG | 15 |
| | (SEQ ID NO: 1261) | |
| hs088406513 | GGATGCCAATGTTCCACAGTGGG | 15 |
| | (SEQ ID NO: 1262) | |
| hs088406685 | CCGGCATCAGAAAACAACACTGG | 15 |
| | (SEQ ID NO: 1263) | |
| hs088406192 | TATTATTGCTGCTTGAACAGGGG | 15 |
| | (SEQ ID NO: 1264) | |
| hs088406392 | ACTAAAGAACTTTAAGAAGGAGG | 15 |
| | (SEQ ID NO: 1265) | |
| hs088406532 | CTCGCACTAGTGTCCACCTGTGG | 15 |
| | (SEQ ID NO: 1266) | |
| hs088406214 | GGCATGTGCTTCCTCTAGGAGGG | 15 |
| | (SEQ ID NO: 1267) | |
| hs088406431 | GAGAATAAGGTCCTTAGTGGAGG | 15 |
| | (SEQ ID NO: 1268) | |
| hs088406188 | TGGGAATTTGTGGGGCCGGGAGG | 15 |
| | (SEQ ID NO: 1269) | |
| hs088406654 | TCTGAAGGTGAAGAGCCACTGGG | 15 |
| | (SEQ ID NO: 1270) | |
| hs088406465 | TTTAAAATTGCTTTGCAGAGGGG | 15 |
| | (SEQ ID NO: 1271) | |
| hs088406213 | GGGCATGTGCTTCCTCTAGGAGG | 15 |
| | (SEQ ID NO: 1272) | |
| hs088406688 | GTTCAATACAGAGAGGGAGCCGG | 15 |
| | (SEQ ID NO: 1273) | |
| hs088406411 | TCTCTGTTGCCACTAAAGGTGGG | 15 |
| | (SEQ ID NO: 1274) | |
| hs088406608 | AGAGCAGAGCCCTAAGCAGATGG | 15 |
| | (SEQ ID NO: 1275) | |
| hs088406346 | TGTTCTGCCCTGAAGCCCAATGG | 15 |
| | (SEQ ID NO: 1276) | |
| hs088406295 | CAAGGCACCATGAAGTACCTTGG | 15 |
| | (SEQ ID NO: 1277) | |
| hs088406511 | CAATGTTCCACAGTGGGAGAGGG | 15 |
| | (SEQ ID NO: 1278) | |
| hs088406463 | CTTTTAAAATTGCTTTGCAGAGG | 15 |
| | (SEQ ID NO: 1279) | |
| hs088406539 | CCACCTGTGGCTGTCAGGGGCGG | 15 |
| | (SEQ ID NO: 1280) | |
| hs088406626 | GATCCCTGAGTGCTGTGAAGTGG | 15 |
| | (SEQ ID NO: 1281) | |
| hs088406339 | ATACCAGGGAGCTCCCAAATGGG | 15 |
| | (SEQ ID NO: 1282) | |
| hs088406521 | TCAGTTCCAGGAAGTTCAAGTGG | 15 |
| | (SEQ ID NO: 1283) | |
| hs088406352 | ACTTGATCATGAAAGCTCCTGGG | 15 |
| | (SEQ ID NO: 1284) | |
| hs088406357 | CCAGGATGTCAAATAGTGGATGG | 15 |
| | (SEQ ID NO: 1285) | |
| hs088406416 | AATTACGAGGTATCAGGGGTAGG | 15 |
| | (SEQ ID NO: 1286) | |
| hs088406665 | AATTTGAGCTCCTATCGACTTGG | 15 |
| | (SEQ ID NO: 1287) | |
| hs088406607 | GAAGTCCTGCCATCGTACCCTGG | 15 |
| | (SEQ ID NO: 1288) | |
| hs088406266 | GAGTTATTGAATAAAAACAAAGG | 15 |
| | (SEQ ID NO: 1289) | |
| hs088406391 | AAGACTAAAGAACTTTAAGAAGG | 15 |
| | (SEQ ID NO: 1290) | |
| hs088406173 | TTATTGCAGCTCATTTCTAGGGG | 15 |
| | (SEQ ID NO: 1291) | |
| hs088406683 | CGGCATCAGAAAACAACACTGGG | 15 |
| | (SEQ ID NO: 1292) | |
| hs088406633 | GGTCACTGGACAGCAGCCTAGGG | 15 |
| | (SEQ ID NO: 1293) | |
| hs088406602 | GGCTTTTGTTAACTGTCATGTGG | 15 |
| | (SEQ ID NO: 1294) | |
| hs088406277 | AACATTTGTGGAAAGAACAAGGG | 15 |
| | (SEQ ID NO: 1295) | |
| hs088406281 | GATGCTGAGCAGGGATAGGAAGG | 15 |
| | (SEQ ID NO: 1296) | |
| hs088406402 | CAGTGAGATCTGAAGTCAAGAGG | 15 |
| | (SEQ ID NO: 1297) | |
| hs088406574 | ACTGGTGGTGTGAGTATGCTAGG | 15 |
| | (SEQ ID NO: 1298) | |
| hs088406443 | TTTCCCGGTTATCACAAGGCAGG | 15 |
| | (SEQ ID NO: 1299) | |
| hs088406386 | CTCCACTAGGAGAATAAAGGTGG | 15 |
| | (SEQ ID NO: 1300) | |
| hs088406552 | ATTGTTGTCCAAAAGGGCAGTGG | 15 |
| | (SEQ ID NO: 1301) | |
| hs088406256 | AAGCAACCTGGCTCCAGAGAGGG | 15 |
| | (SEQ ID NO: 1302) | |
| hs088406691 | TATCAAAGCAAACAAATGCATGG | 15 |
| | (SEQ ID NO: 1303) | |
| hs088406234 | GAAGTGGTCAGCAGGCAGAAAGG | 15 |
| | (SEQ ID NO: 1304) | |
| hs088406306 | TCTTTGACACTGCATCATGTTGG | 15 |
| | (SEQ ID NO: 1305) | |
| hs088406148 | GCTCTGCTACCTGGTAATGAAGG | 15 |
| | (SEQ ID NO: 1306) | |
| hs088406225 | GTATGAAAGATCAACACGTTGGG | 15 |
| | (SEQ ID NO: 1307) | |
| hs088406435 | ATACCTTCTCTAGCCCAGCCTGG | 15 |
| | (SEQ ID NO: 1308) | |
| hs088406259 | ACTCAAGTAGACAAGCAACCTGG | 15 |
| | (SEQ ID NO: 1309) | |
| hs088406555 | GAAGGAACACCAATCACTGATGG | 15 |
| | (SEQ ID NO: 1310) | |
| hs088406396 | TCACTTAGAGGACCAACGACTGG | 15 |
| | (SEQ ID NO: 1312) | |
| hs088406618 | AGAGCCACTTCACAGCACTCAGG | 15 |
| | (SEQ ID NO: 1313) | |
| hs088406538 | TGTCCACCTGTGGCTGTCAGGGG | 15 |
| | (SEQ ID NO: 1314) | |
| hs088406138 | AACTGCATGGGGTTCTGCAAAGG | 15 |
| | (SEQ ID NO: 1315) | |
| hs088406405 | TTCTATCCAACATAAAACACTGG | 15 |
| | (SEQ ID NO: 1316) | |
| hs088406191 | GTATTATTGCTGCTTGAACAGGG | 15 |
| | (SEQ ID NO: 1317) | |
| hs088406251 | GAACTGAGTGATCCTGAGAATGG | 15 |
| | (SEQ ID NO: 1318) | |
| hs088406506 | CCCCCCCCCAAAAAAAGTCTGGG | 15 |
| | (SEQ ID NO: 1319) | |
| hs088406140 | CGGACTGTGACTCTTCAGATGGG | 15 |
| | (SEQ ID NO: 1320) | |
| hs088406545 | GTACCGCCCCTGACAGCCACAGG | 15 |
| | (SEQ ID NO: 1321) | |
| hs088406258 | TGAGTCATATTGATTCAAGCAGG | 15 |
| | (SEQ ID NO: 1322) | |
| hs088406546 | AGTGGCCTTTCCCCTTTCTGTGG | 15 |
| | (SEQ ID NO: 1323) | |
| hs088406305 | AATCTAGAGAGGTTGTTTGACGG | 15 |
| | (SEQ ID NO: 1324) | |
| hs088406284 | TGAGGTCCTGCCTGTCTCCAGGG | 15 |
| | (SEQ ID NO: 1325) | |
| hs088406676 | TGACAGTACAGGAGACCAGAGGG | 15 |
| | (SEQ ID NO: 1326) | |
| hs088406300 | TCTGTCTTGGAGACAGGGACAGG | 15 |
| | (SEQ ID NO: 1327) | |
| hs088406216 | GCACATGCCCTTGAAGACATTGG | 15 |
| | (SEQ ID NO: 1328) | |
| hs088406492 | GCTTGGGTTTGGGATTTACCTGG | 15 |
| | (SEQ ID NO: 1329) | |
| hs088406207 | GGTCATTTCCGTAAGCCACCTGG | 15 |
| | (SEQ ID NO: 1330) | |
| hs088406393 | AGAACTTTAAGAAGGAGGCCTGG | 15 |
| | (SEQ ID NO: 1331) | |
| hs088406573 | AGTGTCCCACCCTCACAGACTGG | 15 |
| | (SEQ ID NO: 1332) | |
| hs088406250 | GTTCAGCTACGAGGAGAAGATGG | 15 |
| | (SEQ ID NO: 1333) | |
| hs088406512 | CCAATGTTCCACAGTGGGAGAGG | 15 |
| | (SEQ ID NO: 1334) | |
| hs088406464 | TTTTAAAATTGCTTTGCAGAGGG | 15 |
| | (SEQ ID NO: 1335) | |
| hs088406509 | CCTCTCCCACTGTGGAACATTGG | 15 |
| | (SEQ ID NO: 1336) | |
| hs088406450 | ACTGTTGTAACACACTTGTCAGG | 15 |
| | (SEQ ID NO: 1337) | |
| hs088406260 | AGCAGGAGAACAGACTTTGAAGG | 15 |
| | (SEQ ID NO: 1338) | |
| hs088406139 | TCGGACTGTGACTCTTCAGATGG | 15 |
| | (SEQ ID NO: 1339) | |
| hs088406165 | CTCTTCCCAGACAAAATGCTGGG | 15 |
| | (SEQ ID NO: 1340) | |
| hs088406433 | TCCTTAGTGGAGGTTAAAAAGGG | 15 |
| | (SEQ ID NO: 1341) | |
| hs088406202 | CTTCAACCAGCTTGTCCAGGTGG | 15 |
| | (SEQ ID NO: 1342) | |
| hs088406338 | ATTCCCATTTGGGAGCTCCCTGG | 15 |
| | (SEQ ID NO: 1343) | |
| hs088406144 | GTCCGATTCTTCAAACTGCATGG | 15 |
| | (SEQ ID NO: 1344) | |
| hs088406644 | TAGCTTGCACATGCTGGCTGGGG | 15 |
| | (SEQ ID NO: 1345) | |
| hs088406640 | CCTTGGCCTGCAGTCACTGGAGG | 15 |
| | (SEQ ID NO: 1346) | |
| hs088406476 | CAAACAGGTCTAAACTTAAGTGG | 15 |
| | (SEQ ID NO: 1347) | |
| hs088406472 | AGACCTGTTTGCAAGATGGTGGG | 15 |
| | (SEQ ID NO: 1348) | |
| hs088406377 | TCCCACCTTTATTCTCCTAGTGG | 15 |
| | (SEQ ID NO: 1349) | |
| hs088406292 | CTTTTCAGAGCCCATGACCCTGG | 15 |
| | (SEQ ID NO: 1350) | |
| hs088406423 | TAACTGTTATGAGTATGTTAGGG | 15 |
| | (SEQ ID NO: 1351) | |
| hs088406359 | TTTGACATCCTGGGGGTCATCGG | 15 |
| | (SEQ ID NO: 1352) | |
| hs088406321 | AAATGGATCAAACAAGCAAAAGG | 15 |
| | (SEQ ID NO: 1353) | |
| hs088406177 | CTGAACATTTCAATCAAGAATGG | 15 |
| | (SEQ ID NO: 1354) | |
| hs088406562 | TTTCTGTTTATAAAAGGTGAGGG | 15 |
| | (SEQ ID NO: 1355) | |
| hs088406594 | CAGGGACAGGTGGCTAAAGAGGG | 15 |
| | (SEQ ID NO: 1356) | |
| hs088406553 | ATTGGAATTGTTGTCCAAAAGGG | 15 |
| | (SEQ ID NO: 1357) | |
| hs088406470 | GTTTAGACCTGTTTGCAAGATGG | 15 |
| | (SEQ ID NO: 1358) | |
| hs088406557 | GAATGGGATCCATCAGTGATTGG | 15 |
| | (SEQ ID NO: 1359) | |
| hs088406231 | CTTCCTGTAGGAACAAAGGTTGG | 15 |
| | (SEQ ID NO: 1360) | |
| hs088406145 | AGTGTTCTGATGCCACAGTTAGG | 15 |
| | (SEQ ID NO: 1361) | |
| hs088406243 | CAGTACTGAGGAGGAGAAAAGGG | 15 |
| | (SEQ ID NO: 1362) | |
| hs088406390 | TCTAGGATGACATCACAGTTAGG | 15 |
| | (SEQ ID NO: 1363) | |
| hs088406687 | TCTCTGTATTGAACGGCAATGGG | 15 |
| | (SEQ ID NO: 1364) | |
| hs088406340 | AATACCAGGGAGCTCCCAAATGG | 15 |
| | (SEQ ID NO: 1365) | |
| hs088406567 | TTTCGAGACTTTGACTTGTACGG | 15 |
| | (SEQ ID NO: 1366) | |
| hs088406328 | CTCATTTTCTGTCGATCAGAAGG | 15 |
| | (SEQ ID NO: 1367) | |
| hs088406227 | GGAAATGAGCTACAAGGAAATGG | 15 |
| | (SEQ ID NO: 1368) | |
| hs088406540 | TCAGGGGCGGTACCACAGAAAGG | 15 |
| | (SEQ ID NO: 1369) | |
| hs088406686 | CTCTCTGTATTGAACGGCAATGG | 15 |
| | (SEQ ID NO: 1370) | |
| hs088406582 | TATTCAAGTCGGACTGAACTAGG | 15 |
| | (SEQ ID NO: 1371) | |
| hs088406221 | GACAGTGACAGCACTAAAAAGGG | 15 |
| | (SEQ ID NO: 1372) | |
| hs088406171 | TGCAGCTCATTTCTAGGGGGTGG | 15 |
| | (SEQ ID NO: 1373) | |
| hs088406493 | GCACATCTATTATTTAGAAAAGG | 15 |
| | (SEQ ID NO: 1374) | |
| hs088406387 | ACTCTCCACTAGGAGAATAAAGG | 15 |
| | (SEQ ID NO: 1375) | |
| hs088406242 | GCAGTACTGAGGAGGAGAAAAGG | 15 |
| | (SEQ ID NO: 1376) | |
| hs088406414 | TATCTTACATCTTTCAGTGATGG | 15 |
| | (SEQ ID NO: 1377) | |
| hs088406382 | AATAAAGGTGGGAGAACAAAGGG | 15 |
| | (SEQ ID NO: 1378) | |
| hs088406159 | AGAGAGTGGGATGAGTCTGGGGG | 15 |
| | (SEQ ID NO: 1379) | |
| hs088406679 | CCAGTGTTGTTTTCTGATGCCGG | 15 |
| | (SEQ ID NO: 1380) | |
| hs088406241 | CCCCCAACCTTTGTTCCTACAGG | 15 |
| | (SEQ ID NO: 1381) | |
| hs088406164 | GACAAAATGCTGGGAGAGAGTGG | 15 |
| | (SEQ ID NO: 1382) | |
| hs088406174 | GTTATTGCAGCTCATTTCTAGGG | 15 |
| | (SEQ ID NO: 1383) | |
| hs088406519 | AGTGGGACTGTTTTAAGAAAAGG | 15 |
| | (SEQ ID NO: 1384) | |
| hs088406486 | TCTAAATAATAGATGTGCTTGGG | 15 |
| | (SEQ ID NO: 1385) | |
| hs088406395 | AAGGAGGCCTGGTCACTTAGAGG | 15 |
| | (SEQ ID NO: 1386) | |
| hs088406642 | GCCAGCATGTGCAAGCTATTAGG | 15 |
| | (SEQ ID NO: 1387) | |
| hs088406429 | AGAGGTTTCAGAGGAGAATAAGG | 15 |
| | (SEQ ID NO: 1388) | |
| hs088406482 | AAAATGTCTTTACTTCTAGAAGG | 15 |
| | (SEQ ID NO: 1389) | |
| hs088406437 | GCTGCCTGCCTTGTGATAACCGG | 15 |
| | (SEQ ID NO: 1390) | |
| hs088406646 | AATAGCTTGCACATGCTGGCTGG | 15 |
| | (SEQ ID NO: 1391) | |
| hs088406534 | GAGCCTGTCGTTCTGCCATTGGG | 15 |
| | (SEQ ID NO: 1392) | |
| hs088406556 | GTGATTGGTGTTCCTTCAATTGG | 15 |
| | (SEQ ID NO: 1393) | |
| hs088406503 | CTTTTTTTGGGGGGGGGTGAGGG | 15 |
| | (SEQ ID NO: 1394) | |
| hs088406516 | AGTAAATCATTTGATCAAAAAGG | 15 |
| | (SEQ ID NO: 1395) | |
| hs088406158 | GAGTGGGATGAGTCTGGGGGTGG | 15 |
| | (SEQ ID NO: 1396) | |
| hs088406313 | ACTGATTGCATCAGCCAAAAAGG | 15 |
| | (SEQ ID NO: 1397) | |

Construct Sequence

saCas9_4x-gRNA(hs07571799[Sa]; hs07571796[Sa]; hs07571783[Sa];
hs07571778[Sa])
CCATGGGCGCATGCAAATTACGCGCTGTGCTTTGTGGGAAATCACCCTAAACGA
AAAATTTATTCCTCTTTCGAGCCTTATAGTGGCGGCCGGTCTACATCCTGAAGA
TCATTCTTGTGGCAGTAAGGTTTTAGTACTCTGGAAACAGAATCTACTAAAACA
AGGCAAAATGCCGTGTTTATCTCGTCAACTTGTTGGCGAGATTTTTTGTTAACG
CGCATGCAAATTACGCGCTGTGCTTTGTGGGAAATCACCCTAAACGAAAAATTT
ATTCCTCTTTCGAGCCTTATAGTGGCGGCCGGTCTACATCCTGAATCAAAAGCT
ACCGGTTACCTGGTTTTAGTACTCTGGAAACAGAATCTACTAAAACAAGGCAA
AATGCCGTGTTTATCTCGTCAACTTGTTGGCGAGATTTTTTTCTAGACACACAAA
AAACCAACACACAGATGTAATGAAAATAAAGATATTTTATTAGGCTGATCAGC
GAGCTCTAGGAATTCTTAGGATCCCTTTTTCTTTTTTGCCTGGCCGGCCTTTTTC
GTGGCCGCCGGCCTTTTGCCCTTTTTGATGATCTGAGGGTGCTTCTTAGATTTCA
CTTCATACAGGTTGCCCAGAATGTCTGTGCTGTACTTCTTAATGCTCTGGGTCTT
GGAGGCGATTGTCTTAATGATCCTGGGGGGCCTCTTGTCGTTCATGTTTTCCAG
GTACTCGCGGTAGGTGATGTCGATCATGTTCACTTCGATCCGGTTCAGCAGGTC
GTTGTTCACGCCGATCACTCTATACAGCTCGCCGTTGATCTTGATCAGATCGTTG
TTGTAGAAGGAGGCGATAAACTCGGCCTGGTTGCTGATCTTCTTCAGCTTCTTA
GCTTCCTCATAGCACTTGCTATTCACTTCGTAGTAGTTTTCTTTTTTGATCACATC
CAGATTCTTCACGGTCACGAACTTGTACACGCCATTGTCCAGGTACACGTCGAA
TCTGTAGGGCTTCAGGGACAGCTTCACGACCTTGTTTCTGCTGTTGGGGTAGTC
GTCGGTGATGTCCAGATGGGCGTTCAGTTTGTTGCCGTAATACTTAATCTTCTTG
ATCACGGGGCCGTTGTCCTTTTTGGAGTACTTGGTCAGGTAGTTCCCGGTTTCCT
CGTAGTACTTGTACAGGGGATTCTTCTCGTCGCCGTACTGTTCCATAATCAGCTT
CAGTTTCTGGTAGGTCTGGGGGTCGTGGTGGTACATCAGCAGCTTTTCGGGGCT
CTTGTTGATCAGCTTTTTCAGCTTGTCATTGTCCTTGTCGTACAGGCCGTTCAGA
TTGTTCACGATCAGGGTGTTGCCCTTGTCGTCCTTCCGGGTGGAGTACAGGGTG
TCGTTAATCAGCTCTCTATTAGGCTTCTTGTCCACCCGGTGGCTGTACTTGTAGT
CCTTGAAGTCCTTAATGTGCTTGATCTGGTGGGGGGTGATGAAGATCTCTTTGT
ACTCCTGCTCGGTTTCGATCTCGGGCATGCTCTCGGCCTGCTTTTCCTCGAACAT
CTGGTTTTCCATCACTTTTTTGGCCTTGTCCAGTTTCTTCCACTCTTTGAAGATGA
AATCGGCGTTGGCAATGATCAGGGCGTCCTCGGCGTGGTGCTTGTACCCCTTGT
TCCGCTCTTTCTTAAACTTCCACTTCCGCCGCAGAAAGCTGGTGAAGCCGCCAT
TGATGGACTTCACTTTCACGTCCAGGTTGTTCACTCTGAAGTAGCTCCGCAGCA
GGTTCATCAGGCCTCTGGTGGCGTATCTGGTATCCACCAGGTTCCGGTTGATGA
AGTCTTTCTGCACGGAGAACCTGTTGATGTCCCGTTCTTCCAGCAGATACTCTTT
CTTGGTCTTGCTGATTCTGCCCTTGCCCTTGGCCAGATTCAGGATGTGCTTCTTG
AAGGTTTCGTAGCTGATCTTGCTGTCGCTGCTGCTCAGGTACTGGAATGGGGTC
CGGTTGCCCTTCTTGCTGTTTTCTTCCTGCTTCACGAGCACCTTGTTGTTGAAGC
TGTTGTCGAAGGACACGCTTCTGGGGATGATGTGGTCCACCTCATAGTTGAAGG
GGTTGTTCAGCAGATCTTCCAGAGGGATGGCTTCCAGGCTGTACAGGCACTTGC
CTTCCTGCATGTCGTGCAGCTTGATCTTCTCGATCAGGTACTTGGCGTTCTCTTT
GCCGGTGGTCCGGATGATTTCCTCGATCCGCTCGTTGGTCTGCCGGTTCCGCTTC
TGCATCTCGTTGATCATTTTCTGGGCGTCCTTGGAGTTCTTCTCGCGGGCCAGCT
CGATAATGATGTCGTTGGGCAGGCCGTACTTCTTGATGATGGCGTTGATCACTT
TGATGCTCTGGATGAAGCTTCTCTTCACGACGGGGCTCAGGATGAAGTCGTCCA
CCAGGGTGGTGGGGATCTCTTTCTGCTGGGACAGGTCCACCTTCTTGGGCACCA
GCTTCAGCCGGTTGAAGATAGCGATCTGGTTGTCGTTGGTGTGCCACAGCTCGT
CCAGGATCAGGTTGATGGCCTTCAGGCTCAGGTTGTGGGTGCCGGTATAGCCCT
TCAGATTAGAGATCTGCTCGATCTCTTCCTGGGTCAGCTCGGAGTTCAGATTGG
TCAGTTCTTCCTGGATGTCCTCGCTGCTCTGGTAGATGGTCAGGATCTTGGCAAT
CTGATCCAGCAGCTCGGCGTTCTCAATAATCTCTTTCCGGGCGGTAATGTCCTT
GATGTCGTGGTACACCTTCAGGTTGGTGAACTCGGGCTTGCCGGTGCTGGTCAC
TCTGTAGCCCTTAATATCCTCTTCGTTCACGAGGATTTCTTTGGCGATCTGCTTC
AGGGTGGGCTTCTTCTTCTGCTTGAACACGTTCTCGATGATCTGGAACTTCTCGT
AATATTCCAGCTTCTCGTTCTCGTCCCTGGTGATCACGAGATTGTTCAGGTCGTT
CAGGGCGTTGTACAGGTCGGCGTTGTAGGCGTACTTCACGCTCCGCAGTTCCTC
GGGGAAGTAGGTGCAGTGGCCCATCAGCATCTCGTACCATTCTTTGATGTCCTT
CCAGCCGAAGGGGCTGCCCTCGCCAGGTCCCTCATAGTAGGTCCGCCGGGTTTC
CAGCAGGTCGATGTAGGTGTCGATGAAGCTCTGGTCCAGCTGGTGGTAGGCCTT
CTGCACCTTCAGCAGCTGTTTGGCTTCTTTCACGTAGTCGCTGGTCTTGAATCTG
TTGATGCTGCCCCGCACTTCGCCGTCTTTCTTCAGCCGTTCCAGCTGCAGTTCGG
CCACGTATTTCTCTTCCAGGGCCTTGCTGTTCCGGCTGATCTGCTCTTTGGTGGA
CAGCTCGTTGCCGGTGTCCTCTTCCACCTCGTTCACGTTGTGCACGCCTCTTCTC
TTGGCCAGGTGCAGCAGGGCGGCAGAGAACTCTTCCTCGCTCAGCTTCTGGCTC
AGGCCCTTCACTCTGGCCTCGTAGGGGTTGATGCCGCTCAGCTCGCTGTGGTCG
GTCAGCAGGTTGTAGTCGAACAGCAGCTTCTTCACTCTCTGGATTCTATGCCGC
CTCCGCCGCTTCAGCCTTCTGGCGCCTCTCTTGCTCCGCCTGCCCTCGTTGTTTT
CCACGTTGGCCTCTTTGAACAGCCGCACGCCGGCATCGATCACGTCCCGTGTCT
CGTAGTCGATGATGCCGTAGCCCACGCTGGTGATGCCGATGTCCAGGCCCAGG
ATGTAGTTCCGCTTGGCTGCTGGGACTCCGTGGATACCGACCTTCCGCTTCTTCT
TTGGGGCCATGTGGCGGCTCTTGAAGGACGACGTCATCATCCCTTGCCCGGATG
CGCGGGCTTCTTGTCTAGCACAGGAGCCTGGGGTAGAGCGCATGCAAATTACG
CGCTGTGCTTTGTGGGAAATCACCCTAAACGAAAAATTTATTCCTCTTTCGAGC
CTTATAGTGGCGGCCGGTCTACATCCTGAAGATCTTATTCTACTCCTGTGAGTTT
TAGTACTCTGGAAACAGAATCTACTAAAACAAGGCAAAATGCCGTGTTTATCTC
GTCAACTTGTTGGCGAGATTTTTTGATATCGCGCATGCAAATTACGCGCTGTGC
TTTGTGGGAAATCACCCTAAACGAAAAATTTATTCCTCTTTCGAGCCTTATAGT
GGCGGCCGGTCTACATCCTGAAAACGTTGTTCTGAGTAGCTTTGTTTTAGTACT
CTGGAAACAGAATCTACTAAAACAAGGCAAAATGCCGTGTTTATCTCGTCAACT
TGTTGGCGAGATTTTTTCTCGAGGCGGCCGC (SEQ ID NO: 110)

Self-complementary AAV Vectors

pscAAV_DLK(mm079)
AAAGCTTCCCGGGGGGATCTGGGCCACTCCCTCTCTGCGCGCTCGCTCGCTCAC
TGAGGCCGGGCGACCAAAGGTCGCCCGACGCCCGGGCTTTGCCCGGGCGGCCT
CAGTGAGCGAGCGAGCGCGCAGAGAGGGAGTGGCCAACTCCATCACTAGGGGT
TCCTGGAGGGGTGGAGTCGTGACCTAGGGCGGCCGCTAAGATACATTGATGAG
TTTGGACAAACCACAACTAGAATGCAGTGAAAAAAATGCTTTATTTGTGAAATT
TGTGATGCTATTGCTTTATTTGTAACCATTATAAGCTGCAATAAACAAGTTAAC
AACAACAATTGCATTCATTTTATGTTTCAGGTTCAGGGGGAGGTGTGGGAGGTT
TTTTAAAGCAAGTAAAACCTCTACAAATGTGGTATGGCTGATTATGATCAGTTA
TCTAGATTTACTTATGTAGCTCGTCCATGCCGCCGGTGGAGTGGCGGCCCTCGG
CGCGTTCGTACTGTTCCACGATGGTGTAGTCCTCGTTGTGGGAGGTGATGTCCA
ACTTGATGTTGACGTTGTAGGCGCCGGGCAGCTGCACGGGCTTCTTGGCCTTGT
AGGTGGTCTTGACCTCAGCGTCGTAGTGGCCGCCGTCCTTCAGCTTCAGCCTCT
GCTTGATCTCGCCCTTCAGGGCGCCGTCCTCGGGGTACATCCGCTCGGAGGAGG
CCTCCCAGCCCATGGTCTTCTTCTGCATTACGGGGCCGTCGGAGGGGAAGTTGG
TGCCGCGCAGCTTCACCTTGTAGATGAACTCGCCGTCCTGCAGGGAGGAGTCCT
GGGTCACGGTCACCACGCCGCCGTCCTCGAAGTTCATCACGCGCTCCCACTTGA
AGCCCTCGGGGAAGGACAGCTTCAAGTAGTCGGGGATGTCGGCGGGGTGCTTC
ACGTAGGCCTTGGAGCCGTACATGAACTGAGGGGACAGGATGTCCCAGGCGAA
GGGCAGGGGGCCACCCTTGGTCACCTTCAGCTTGGCGGTCTGGGTGCCCTCGTA
GGGGCGGCCCTCGCCCTCGCCCTCGATCTCGAACTCGTGGCCGTTCACGGAGCC
CTCCATGTGCACCTTGAAGCGCATGAACTCCTTGATGATGGCCATGTTATCCTC
CTCGCCCTTGCTCACCATGGTGGCGACCGGTGGATCCTTAGAGCTAGTGTACTT
GGTAACTGCCTTAGTGCCCTCGGACACAGCATGCTTAGCCAGCTCCCCAGGCAG
CAGCAGGCGCACAGCCGTCTGAATCTCCCTGGAGGTGATGGTCGAGCGCTTATT
GTAGTGAGCCAGGCGAGAAGCCTCGCCCGCGATGCGCTCGAAGATGTCGTTGA
CGAAGGAGTTCATGATCCCCATGGCCTTGGATGAGATGCCGGTGTCGGGGTGG
ACCTGCTTCAGAACCTTGTACACATAGATAGAATAGCTCTCCTTGCGGCTGCGC
TTACGCTTCTTACCATCCTTCTTCTGCGCCTTAGTGATAGCCTTCTTAGAACCCT
TTTTAGGGGCTGGAGCAGACTTAGAGGGTTCAGGCATGGTGGCGGCCGCTCTTG
AAGGACGACGTCATCATCCCTTGCCCGGATGCGCGGGCTTCTTGTCTAGCACAG
GAGCCTGGGGTAGAGCGCATGCAAATTACGCGCTGTGCTTTGTGGGAAATCAC
CCTAAACGAAAAATTTATTCCTCTTTCGAGCCTTATAGTGGCGGCCGGTCTACA
TCCTGAAGAAGAAGGTTCGAGATCTCAGTTTTAGAGCTAGAAATAGCAAGTTA
AAATAAGGCTAGTCCGTTATCAACTTGAAAAAGTGGCACCGAGTCGGTGCTTTT
TTTCTAGACCCAGCTTTCTTGTACAAAGTTGGCGGCCGCACTAGTCCACTCCCTC
TCTGCGCGCTCGCTCGCTCACTGAGGCCGGGCGACCAAAGGTCGCCCGACGCC
CGGGCTTTGCCCGGGCGGCCTCAGTGAGCGAGCGAGCGCGCAGAGAGGGACAG
ATCCGGGCCCGCATGCGTCGACAATTCACTGGCCGTCGTTTTACAACGTCGTGA
CTGGGAAAACCCTGGCGTTACCCAACTTAATCGCCTTGCAGCACATCCCCCTTT
CGCCAGCTGGCGTAATAGCGAAGAGGCCCGCACCGATCGCCCTTCCCAACAGT
TGCGCAGCCTGAATGGCGAATGGCGCCTGATGCGGTATTTTCTCCTTACGCATC
TGTGCGGTATTTCACACCGCATATGGTGCACTCTCAGTACAATCTGCTCTGATG
CCGCATAGTTAAGCCAGCCCCGACACCCGCCAACACCCGCTGACGCGCCCTGA
CGGGCTTGTCTGCTCCCGGCATCCGCTTACAGACAAGCTGTGACCGTCTCCGGG
AGCTGCATGTGTCAGAGGTTTTCACCGTCATCACCGAAACGCGCGAGACGAAA
GGGCCTCGTGATACGCCTATTTTTATAGGTTAATGTCATGATAATAATGGTTTCT
TAGACGTCAGGTGGCACTTTTCGGGGAAATGTGCGCGGAACCCCTATTTGTTTA
TTTTTCTAAATACATTCAAATATGTATCCGCTCATGAGACAATAACCCTGATAA
ATGCTTCAATAATATTGAAAAAGGAAGAGTATGAGTATTCAACATTTCCGTGTC
GCCCTTATTCCCTTTTTTGCGGCATTTTGCCTTCCTGTTTTTGCTCACCCAGAAAC
GCTGGTGAAAGTAAAAGATGCTGAAGATCAGTTGGGTGCACGAGTGGGTTACA
TCGAACTGGATCTCAACAGCGGTAAGATCCTTGAGAGTTTTCGCCCCGAAGAAC
GTTTTCCAATGATGAGCACTTTTAAAGTTCTGCTATGTGGCGCGGTATTATCCCG
TATTGACGCCGGGCAAGAGCAACTCGGTCGCCGCATACACTATTCTCAGAATG
ACTTGGTTGAGTACTCACCAGTCACAGAAAAGCATCTTACGGATGGCATGACA
GTAAGAGAATTATGCAGTGCTGCCATAACCATGAGTGATAACACTGCGGCCAA
CTTACTTCTGACAACGATCGGAGGACCGAAGGAGCTAACCGCTTTTTTGCACAA
CATGGGGGATCATGTAACTCGCCTTGATCGTTGGGAACCGGAGCTGAATGAAG
CCATACCAAACGACGAGCGTGACACCACGATGCCTGTAGCAATGGCAACAACG
TTGCGCAAACTATTAACTGGCGAACTACTTACTCTAGCTTCCCGGCAACAATTA
ATAGACTGGATGGAGGCGGATAAAGTTGCAGGACCACTTCTGCGCTCGGCCCT
TCCGGCTGGCTGGTTTATTGCTGATAAATCTGGAGCCGGTGAGCGTGGGTCTCG
CGGTATCATTGCAGCACTGGGGCCAGATGGTAAGCCCTCCCGTATCGTAGTTAT
CTACACGACGGGGAGTCAGGCAACTATGGATGAACGAAATAGACAGATCGCTG
AGATAGGTGCCTCACTGATTAAGCATTGGTAACTGTCAGACCAAGTTTACTCAT
ATATACTTTAGATTGATTTAAAACTTCATTTTTAATTTAAAAGGATCTAGGTGA
AGATCCTTTTTGATAATCTCATGACCAAAATCCCTTAACGTGAGTTTTCGTTCCA
CTGAGCGTCAGACCCCGTAGAAAAGATCAAAGGATCTTCTTGAGATCCTTTTTT
TCTGCGCGTAATCTGCTGCTTGCAAACAAAAAAACCACCGCTACCAGCGGTGGT
TTGTTTGCCGGATCAAGAGCTACCAACTCTTTTTCCGAAGGTAACTGGCTTCAG
CAGAGCGCAGATACCAAATACTGTTCTTCTAGTGTAGCCGTAGTTAGGCCACCA
CTTCAAGAACTCTGTAGCACCGCCTACATACCTCGCTCTGCTAATCCTGTTACC
AGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACAC
AGCCCAGCTTGGAGCGAACGACCTACACCGAACTGAGATACCTACAGCGTGAG
CTATGAGAAAGCGCCACGCTTCCCGAAGGGAGAAAGGCGGACAGGTATCCGGT
AAGCGGCAGGGTCGGAACAGGAGAGCGCACGAGGGAGCTTCCAGGGGGAAAC
GCCTGGTATCTTTATAGTCCTGTCGGGTTTCGCCACCTCTGACTTGAGCGTCGAT
TTTTGTGATGCTCGTCAGGGGGGCGGAGCCTATGGAAAAACGCCAGCAACGCG
GCCTTTTTACGGTTCCTGGCCTTTTGCTGGCCTTTTGCTCACATGTTCTTTCCTGC
GTTATCCCCTGATTCTGTGGATAACCGTATTACCGCCTTTGAGTGAGCTGATAC
CGCTCGCCGCAGCCGAACGACCGAGCGCAGCGAGTCAGTGAGCGAGGAAGCG
GAAGAGCGCCCAATACGCAAACCGCCTCTCCCCGCGCGTTGGCCGATTCATTAA
TGCAGCTGGCACGACAGGTTTCCCGACTGGAAAGCGGGCAGTGAGCGCAACGC
AATTAATGTGAGTTAGCTCACTCATTAGGCACCCCAGGCTTTACACTTTATGCTT
CCGGCTCGTATGTTGTGTGGAATTGTGAGCGGATAACAATTTCACACAGGAAAC
AGCTATGACCATGATTACGCCAAGCTCTCGAGATCTAG (SEQ ID NO: 1398)
pscAAV_LZK(GFP)
AAAGCTTCCCGGGGGGATCTGGGCCACTCCCTCTCTGCGCGCTCGCTCGCTCAC
TGAGGCCGGGCGACCAAAGGTCGCCCGACGCCCGGGCTTTGCCCGGGCGGCCT
CAGTGAGCGAGCGAGCGCGCAGAGAGGGAGTGGCCAACTCCATCACTAGGGGT
TCCTGGAGGGGTGGAGTCGTGACCTAGGGCGGCCGCTAAGATACATTGATGAG
TTTGGACAAACCACAACTAGAATGCAGTGAAAAAAATGCTTTATTTGTGAAATT
TGTGATGCTATTGCTTTATTTGTAACCATTATAAGCTGCAATAAACAAGTTAAC
AACAACAATTGCATTCATTTTATGTTTCAGGTTCAGGGGGAGGTGTGGGAGGTT
TTTTAAAGCAAGTAAAACCTCTACAAATGTGGTATGGCTGATTATGATCAGTTA
TCTAGATTTACTTGTACAGCTCGTCCATGCCGAGAGTGATCCCGGCGGCGGTCA
CGAACTCCAGCAGGACCATGTGATCGCGCTTCTCGTTGGGGTCTTTGCTCAGGG
CGGACTGGGTGCTCAGGTAGTGGTTGTCGGGCAGCAGCACGGGGCCGTCGCCG
ATGGGGGTGTTCTGCTGGTAGTGGTCGGCGAGCTGCACGCTGCCGTCCTCGATG
TTGTGGCGGATCTTGAAGTTCACCTTGATGCCGTTCTTCTGCTTGTCGGCCATGA
TATAGACGTTGTGGCTGTTGTAGTTGTACTCCAGCTTGTGCCCCAGGATGTTGC
CGTCCTCCTTGAAGTCGATGCCCTTCAGCTCGATGCGGTTCACCAGGGTGTCGC
CCTCGAACTTCACCTCGGCGCGGGTCTTGTAGTTGCCGTCGTCCTTGAAGAAGA
TGGTGCGCTCCTGGACGTAGCCTTCGGGCATGGCGGACTTGAAGAAGTCGTGCT
GCTTCATGTGGTCGGGGTAGCGGCTGAAGCACTGCACGCCGTAGGTCAGGGTG
GTCACGAGGGTGGGCCAGGGCACGGGCAGCTTGCCGGTGGTGCAGATGAACTT
CAGGGTCAGCTTGCCGTAGGTGGCATCGCCCTCGCCCTCGCCGGACACGCTGAA
CTTGTGGCCGTTTACGTCGCCGTCCAGCTCGACCAGGATGGGCACCACCCCGGT
GAACAGCTCCTCGCCCTTGCTCACCATGGTGGCGACCGGTGGATCCTTAGAGCT
AGTGTACTTGGTAACTGCCTTAGTGCCCTCGGACACAGCATGCTTAGCCAGCTC
CCCAGGCAGCAGCAGGCGCACAGCCGTCTGAATCTCCCTGGAGGTGATGGTCG
AGCGCTTATTGTAGTGAGCCAGGCGAGAAGCCTCGCCCGCGATGCGCTCGAAG
ATGTCGTTGACGAAGGAGTTCATGATCCCCATGGCCTTGGATGAGATGCCGGTG
TCGGGGTGGACCTGCTTCAGAACCTTGTACACATAGATAGAATAGCTCTCCTTG
CGGCTGCGCTTACGCTTCTTACCATCCTTCTTCTGCGCCTTAGTGATAGCCTTCT
TAGAACCCTTTTTAGGGGCTGGAGCAGACTTAGAGGGTTCAGGCATGGTGGCG
GCCGCTCTTGAAGGACGACGTCATCATCCCTTGCCCGGATGCGCGGGCTTCTTG
TCTAGCACAGGAGCCTGGGGTAGAGCGCATGCAAATTACGCGCTGTGCTTTGTG
GGAAATCACCCTAAACGAAAAATTTATTCCTCTTTCGAGCCTTATAGTGGCGGC
CGGTCTACATCCTGAAGCCGACACCCCAAATGATGGGTTTTAGAGCTAGAAAT
AGCAAGTTAAAATAAGGCTAGTCCGTTATCAACTTGAAAAAGTGGCACCGAGT
CGGTGCTTTTTTTCTAGACCCAGCTTTCTTGTACAAAGTTGGCGGCCGCACTAGT
CCACTCCCTCTCTGCGCGCTCGCTCGCTCACTGAGGCCGGGCGACCAAAGGTCG
CCCGACGCCCGGGCTTTGCCCGGGCGGCCTCAGTGAGCGAGCGAGCGCGCAGA
GAGGGACAGATCCGGGCCCGCATGCGTCGACAATTCACTGGCCGTCGTTTTACA
ACGTCGTGACTGGGAAAACCCTGGCGTTACCCAACTTAATCGCCTTGCAGCACA
TCCCCCTTTCGCCAGCTGGCGTAATAGCGAAGAGGCCCGCACCGATCGCCCTTC
CCAACAGTTGCGCAGCCTGAATGGCGAATGGCGCCTGATGCGGTATTTTCTCCT
TACGCATCTGTGCGGTATTTCACACCGCATATGGTGCACTCTCAGTACAATCTG
CTCTGATGCCGCATAGTTAAGCCAGCCCCGACACCCGCCAACACCCGCTGACG
CGCCCTGACGGGCTTGTCTGCTCCCGGCATCCGCTTACAGACAAGCTGTGACCG
TCTCCGGGAGCTGCATGTGTCAGAGGTTTTCACCGTCATCACCGAAACGCGCGA
GACGAAAGGGCCTCGTGATACGCCTATTTTTATAGGTTAATGTCATGATAATAA
TGGTTTCTTAGACGTCAGGTGGCACTTTTCGGGGAAATGTGCGCGGAACCCCTA
TTTGTTTATTTTTCTAAATACATTCAAATATGTATCCGCTCATGAGACAATAACC
CTGATAAATGCTTCAATAATATTGAAAAAGGAAGAGTATGAGTATTCAACATTT
CCGTGTCGCCCTTATTCCCTTTTTTGCGGCATTTTGCCTTCCTGTTTTTGCTCACC
CAGAAACGCTGGTGAAAGTAAAAGATGCTGAAGATCAGTTGGGTGCACGAGTG
GGTTACATCGAACTGGATCTCAACAGCGGTAAGATCCTTGAGAGTTTTCGCCCC
GAAGAACGTTTTCCAATGATGAGCACTTTTAAAGTTCTGCTATGTGGCGCGGTA
TTATCCCGTATTGACGCCGGGCAAGAGCAACTCGGTCGCCGCATACACTATTCT
CAGAATGACTTGGTTGAGTACTCACCAGTCACAGAAAAGCATCTTACGGATGG
CATGACAGTAAGAGAATTATGCAGTGCTGCCATAACCATGAGTGATAACACTG
CGGCCAACTTACTTCTGACAACGATCGGAGGACCGAAGGAGCTAACCGCTTTTT
TGCACAACATGGGGGATCATGTAACTCGCCTTGATCGTTGGGAACCGGAGCTG
AATGAAGCCATACCAAACGACGAGCGTGACACCACGATGCCTGTAGCAATGGC
AACAACGTTGCGCAAACTATTAACTGGCGAACTACTTACTCTAGCTTCCCGGCA
ACAATTAATAGACTGGATGGAGGCGGATAAAGTTGCAGGACCACTTCTGCGCT
CGGCCCTTCCGGCTGGCTGGTTTATTGCTGATAAATCTGGAGCCGGTGAGCGTG
GGTCTCGCGGTATCATTGCAGCACTGGGGCCAGATGGTAAGCCCTCCCGTATCG
TAGTTATCTACACGACGGGGAGTCAGGCAACTATGGATGAACGAAATAGACAG
ATCGCTGAGATAGGTGCCTCACTGATTAAGCATTGGTAACTGTCAGACCAAGTT
TACTCATATATACTTTAGATTGATTTAAAACTTCATTTTTAATTTAAAAGGATCT
AGGTGAAGATCCTTTTTGATAATCTCATGACCAAAATCCCTTAACGTGAGTTTT
CGTTCCACTGAGCGTCAGACCCCGTAGAAAAGATCAAAGGATCTTCTTGAGATC
CTTTTTTTCTGCGCGTAATCTGCTGCTTGCAAACAAAAAAACCACCGCTACCAG
CGGTGGTTTGTTTGCCGGATCAAGAGCTACCAACTCTTTTTCCGAAGGTAACTG
GCTTCAGCAGAGCGCAGATACCAAATACTGTTCTTCTAGTGTAGCCGTAGTTAG
GCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACT
CAAGACGATAGTTACCGGATAAGGCGCAGCGGTCGGGCTGAACGGGGGGTTCG
TGCACACAGCCCAGCTTGGAGCGAACGACCTACACCGAACTGAGATACCTACA
GCGTGAGCTATGAGAAAGCGCCACGCTTCCCGAAGGGAGAAAGGCGGACAGG
TATCCGGTAAGCGGCAGGGTCGGAACAGGAGAGCGCACGAGGGAGCTTCCAG
GGGGAAACGCCTGGTATCTTTATAGTCCTGTCGGGTTTCGCCACCTCTGACTTG
AGCGTCGATTTTTGTGATGCTCGTCAGGGGGGCGGAGCCTATGGAAAAACGCC
AGCAACGCGGCCTTTTTACGGTTCCTGGCCTTTTGCTGGCCTTTTGCTCACATGT
TCTTTCCTGCGTTATCCCCTGATTCTGTGGATAACCGTATTACCGCCTTTGAGTG
AGCTGATACCGCTCGCCGCAGCCGAACGACCGAGCGCAGCGAGTCAGTGAGCG
AGGAAGCGGAAGAGCGCCCAATACGCAAACCGCCTCTCCCCGCGCGTTGGCCG
ATTCATTAATGCAGCTGGCACGACAGGTTTCCCGACTGGAAAGCGGGCAGTGA
GCGCAACGCAATTAATGTGAGTTAGCTCACTCATTAGGCACCCCAGGCTTTACA
CTTTATGCTTCCGGCTCGTATGTTGTGTGGAATTGTGAGCGGATAACAATTTCA
CACAGGAAACAGCTATGACCATGATTACGCCAAGCTCTCGAGATCTAG (SEQ ID
NO: 1399)
pscAAV_dual(DLK + LZK)
AAAGCTTCCCGGGGGGATCTGGGCCACTCCCTCTCTGCGCGCTCGCTCGCTCAC
TGAGGCCGGGCGACCAAAGGTCGCCCGACGCCCGGGCTTTGCCCGGGCGGCCT
CAGTGAGCGAGCGAGCGCGCAGAGAGGGAGTGGCCAACTCCATCACTAGGGGT
TCCTGGAGGGGTGGAGTCGTGACCTAGGGCGGCCGCCTGCAATATTTGCATGTC
GCTATGTGTTCTGGGAAATCACCATAAACGTGAAATGTCTTTGGATTTGGGAGT
CTTATAAGTTCTGTATGAGACCACTTTTTCCCGCCGACACCCCAAATGATGGGT
TTTAGAGCTAGAAATAGCAAGTTAAAATAAGGCTAGTCCGTTATCAACTTGAA
AAAGTGGCACCGAGTCGGTGCTTTTTTTAAGATACATTGATGAGTTTGGACAAA
CCACAACTAGAATGCAGTGAAAAAAATGCTTTATTTGTGAAATTTGTGATGCTA
TTGCTTTATTTGTAACCATTATAAGCTGCAATAAACAAGTTAACAACAACAATT
GCATTCATTTTATGTTTCAGGTTCAGGGGGAGGTGTGGGAGGTTTTTTAAAGCA
AGTAAAACCTCTACAAATGTGGTATGGCTGATTATGATCAGTTATCTAGATTTA
CTTATGTAGCTCGTCCATGCCGCCGGTGGAGTGGCGGCCCTCGGCGCGTTCGTA
CTGTTCCACGATGGTGTAGTCCTCGTTGTGGGAGGTGATGTCCAACTTGATGTT
GACGTTGTAGGCGCCGGGCAGCTGCACGGGCTTCTTGGCCTTGTAGGTGGTCTT
GACCTCAGCGTCGTAGTGGCCGCCGTCCTTCAGCTTCAGCCTCTGCTTGATCTC
GCCCTTCAGGGCGCCGTCCTCGGGGTACATCCGCTCGGAGGAGGCCTCCCAGC
CCATGGTCTTCTTCTGCATTACGGGGCCGTCGGAGGGGAAGTTGGTGCCGCGCA
GCTTCACCTTGTAGATGAACTCGCCGTCCTGCAGGGAGGAGTCCTGGGTCACGG
TCACCACGCCGCCGTCCTCGAAGTTCATCACGCGCTCCCACTTGAAGCCCTCGG
GGAAGGACAGCTTCAAGTAGTCGGGGATGTCGGCGGGGTGCTTCACGTAGGCC
TTGGAGCCGTACATGAACTGAGGGGACAGGATGTCCCAGGCGAAGGGCAGGG
GGCCACCCTTGGTCACCTTCAGCTTGGCGGTCTGGGTGCCCTCGTAGGGGCGGC
CCTCGCCCTCGCCCTCGATCTCGAACTCGTGGCCGTTCACGGAGCCCTCCATGT
GCACCTTGAAGCGCATGAACTCCTTGATGATGGCCATGTTATCCTCCTCGCCCT
TGCTCACCATGGTGGCGACCGGTGGATCCTTAGAGCTAGTGTACTTGGTAACTG
CCTTAGTGCCCTCGGACACAGCATGCTTAGCCAGCTCCCCAGGCAGCAGCAGG
CGCACAGCCGTCTGAATCTCCCTGGAGGTGATGGTCGAGCGCTTATTGTAGTGA
GCCAGGCGAGAAGCCTCGCCCGCGATGCGCTCGAAGATGTCGTTGACGAAGGA
GTTCATGATCCCCATGGCCTTGGATGAGATGCCGGTGTCGGGGTGGACCTGCTT
CAGAACCTTGTACACATAGATAGAATAGCTCTCCTTGCGGCTGCGCTTACGCTT
CTTACCATCCTTCTTCTGCGCCTTAGTGATAGCCTTCTTAGAACCCTTTTTAGGG
GCTGGAGCAGACTTAGAGGGTTCAGGCATGGTGGCGGCCGCTCTTGAAGGACG
ACGTCATCATCCCTTGCCCGGATGCGCGGGCTTCTTGTCTAGCACAGGAGCCTG
GGGTAGAGCGCATGCAAATTACGCGCTGTGCTTTGTGGGAAATCACCCTAAAC
GAAAAATTTATTCCTCTTTCGAGCCTTATAGTGGCGGCCGGTCTACATCCTGAA
GAAGAAGGTTCGAGATCTCAGTTTTAGAGCTAGAAATAGCAAGTTAAAATAAG
GCTAGTCCGTTATCAACTTGAAAAAGTGGCACCGAGTCGGTGCTTTTTTTCTAG
ACCCAGCTTTCTTGTACAAAGTTGGCGGCCGCACTAGTCCACTCCCTCTCTGCG
CGCTCGCTCGCTCACTGAGGCCGGGCGACCAAAGGTCGCCCGACGCCCGGGCT
TTGCCCGGGCGGCCTCAGTGAGCGAGCGAGCGCGCAGAGAGGGACAGATCCGG
GCCCGCATGCGTCGACAATTCACTGGCCGTCGTTTTACAACGTCGTGACTGGGA
AAACCCTGGCGTTACCCAACTTAATCGCCTTGCAGCACATCCCCCTTTCGCCAG
CTGGCGTAATAGCGAAGAGGCCCGCACCGATCGCCCTTCCCAACAGTTGCGCA
GCCTGAATGGCGAATGGCGCCTGATGCGGTATTTTCTCCTTACGCATCTGTGCG
GTATTTCACACCGCATATGGTGCACTCTCAGTACAATCTGCTCTGATGCCGCAT
AGTTAAGCCAGCCCCGACACCCGCCAACACCCGCTGACGCGCCCTGACGGGCT
TGTCTGCTCCCGGCATCCGCTTACAGACAAGCTGTGACCGTCTCCGGGAGCTGC
ATGTGTCAGAGGTTTTCACCGTCATCACCGAAACGCGCGAGACGAAAGGGCCT
CGTGATACGCCTATTTTTATAGGTTAATGTCATGATAATAATGGTTTCTTAGACG
TCAGGTGGCACTTTTCGGGGAAATGTGCGCGGAACCCCTATTTGTTTATTTTTCT
AAATACATTCAAATATGTATCCGCTCATGAGACAATAACCCTGATAAATGCTTC
AATAATATTGAAAAAGGAAGAGTATGAGTATTCAACATTTCCGTGTCGCCCTTA
TTCCCTTTTTTGCGGCATTTTGCCTTCCTGTTTTTGCTCACCCAGAAACGCTGGT
GAAAGTAAAAGATGCTGAAGATCAGTTGGGTGCACGAGTGGGTTACATCGAAC
TGGATCTCAACAGCGGTAAGATCCTTGAGAGTTTTCGCCCCGAAGAACGTTTTC
CAATGATGAGCACTTTTAAAGTTCTGCTATGTGGCGCGGTATTATCCCGTATTG
ACGCCGGGCAAGAGCAACTCGGTCGCCGCATACACTATTCTCAGAATGACTTG
GTTGAGTACTCACCAGTCACAGAAAAGCATCTTACGGATGGCATGACAGTAAG
AGAATTATGCAGTGCTGCCATAACCATGAGTGATAACACTGCGGCCAACTTACT
TCTGACAACGATCGGAGGACCGAAGGAGCTAACCGCTTTTTTGCACAACATGG
GGGATCATGTAACTCGCCTTGATCGTTGGGAACCGGAGCTGAATGAAGCCATA
CCAAACGACGAGCGTGACACCACGATGCCTGTAGCAATGGCAACAACGTTGCG
CAAACTATTAACTGGCGAACTACTTACTCTAGCTTCCCGGCAACAATTAATAGA
CTGGATGGAGGCGGATAAAGTTGCAGGACCACTTCTGCGCTCGGCCCTTCCGGC
TGGCTGGTTTATTGCTGATAAATCTGGAGCCGGTGAGCGTGGGTCTCGCGGTAT
CATTGCAGCACTGGGGCCAGATGGTAAGCCCTCCCGTATCGTAGTTATCTACAC
GACGGGGAGTCAGGCAACTATGGATGAACGAAATAGACAGATCGCTGAGATA
GGTGCCTCACTGATTAAGCATTGGTAACTGTCAGACCAAGTTTACTCATATATA
CTTTAGATTGATTTAAAACTTCATTTTTAATTTAAAAGGATCTAGGTGAAGATC
CTTTTTGATAATCTCATGACCAAAATCCCTTAACGTGAGTTTTCGTTCCACTGAG
CGTCAGACCCCGTAGAAAAGATCAAAGGATCTTCTTGAGATCCTTTTTTTCTGC
GCGTAATCTGCTGCTTGCAAACAAAAAAACCACCGCTACCAGCGGTGGTTTGTT
TGCCGGATCAAGAGCTACCAACTCTTTTTCCGAAGGTAACTGGCTTCAGCAGAG
CGCAGATACCAAATACTGTTCTTCTAGTGTAGCCGTAGTTAGGCCACCACTTCA
AGAACTCTGTAGCACCGCCTACATACCTCGCTCTGCTAATCCTGTTACCAGTGG
CTGCTGCCAGTGGCGATAAGTCGTGTCTTACCGGGTTGGACTCAAGACGATAGT
TACCGGATAAGGCGCAGCGGTCGGGCTGAACGGGGGGTTCGTGCACACAGCCC
AGCTTGGAGCGAACGACCTACACCGAACTGAGATACCTACAGCGTGAGCTATG
AGAAAGCGCCACGCTTCCCGAAGGGAGAAAGGCGGACAGGTATCCGGTAAGC
GGCAGGGTCGGAACAGGAGAGCGCACGAGGGAGCTTCCAGGGGGAAACGCCT
GGTATCTTTATAGTCCTGTCGGGTTTCGCCACCTCTGACTTGAGCGTCGATTTTT
GTGATGCTCGTCAGGGGGGCGGAGCCTATGGAAAAACGCCAGCAACGCGGCCT
TTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCT
CGCCGCAGCCGAACGACCGAGCGCAGCGAGTCAGTGAGCGAGGAAGCGGAAG
AGCGCCCAATACGCAAACCGCCTCTCCCCGCGCGTTGGCCGATTCATTAATGCA
GCTGGCACGACAGGTTTCCCGACTGGAAAGCGGGCAGTGAGCGCAACGCAATT
AATGTGAGTTAGCTCACTCATTAGGCACCCCAGGCTTTACACTTTATGCTTCCG
GCTCGTATGTTGTGTGGAATTGTGAGCGGATAACAATTTCACACAGGAAACAG
CTATGACCATGATTACGCCAAGCTCTCGAGATCTAG (SEQ ID NO: 1400)

### Example 2

Provided herein is a AAV-based approach to the treatment of LCA. In contrast to treatments that involve gene transfer, the approach uses CRISPR genome editing technology. CRISPR has been developed in recent years and has very rapidly revolutionized biological research and ushered in a new era for genetic medicine (4, 5). CRISPRs are composed of a bacterial endonuclease and a short RNA that guides this nuclease to a specific cleavage site in the genome. With a customized guide RNA (gRNA), a CRISPR can be programmed to disrupt any human gene or regulatory element, or to delete and replace genomic DNA sequences in a highly-specific fashion. A CRISPR approach to LCA will facilitate the permanent removal of the mutation from the cells at risk for degeneration, and thus cure the disease.

There is one major technical obstacle to employing AAV for CRISPR applications—their size. AAV are small viruses that can package up to 5.2 kb of DNA. A standard CRISPR exceeds this packaging capacity by a significant margin. CRISPRs are composed of the bacterial endonuclease Cas9 and at least one gRNA. The most commonly used Cas9 protein, from S. pyogenes is alone encoded by a 4.1 kb gene. It is impossible to package both CRISPR components—with standard promoters and terminator sequences necessary for expression—into a single virus.

WO2015195621, herein incorporated by reference, discloses a solution to the packaging capacity problem. At the core of this new approach to CRISPR delivery is a compact bidirectional promoter known as H1. A single H1 promoter can efficiently express both Cas9 and gRNA. This unique genetic element allows an assembly composed of any Cas9 gene and multiple gRNAs, optimally four, to be packaged in a single recombinant AAV (FIG. 1), called an AAV-H1-CRISPR. The capacity for added gRNA allows AAV-H1-CRISPR to generate double strand breaks with unmatched site-specificity, thus minimizing the well-known risk of off-target mutagenesis (6, 7).

Provided herein is a safe and durable treatment for LCA10 by a singly-administered AAV-H1-CRISPR therapeutic. LCA10 is caused by mutations in the gene CEP290. This subtype of LCA affects about 2,000 individuals in the western world. This therapeutic contrasts with the current technology being developed by Editas Medicine. Their solution to the packaging capacity problem is to employ a smaller Cas9 gene. However, their vector configuration can engage fewer genomic targets than ours, and lacks a critical feature: the use of four gRNA to provide exquisite targeting sensitivity (as mentioned above), that has been shown to prevent off-target mutagenesis, a significant safety issue (6, 7). The AAV-H1-CRISPR retains this critical feature. The rate of off-target mutations caused by our LCA10 vector is expected to be negligible.
1. Assembly of viral construct. To facilitate the rapid assembly of viral vectors from modular components in a commercial lab setting, synthesize modular cassettes will be synthesized that will be reusable for subsequent targets. The vector for LCA10 will be assembled from these generic modules and four CEP290-specific H1-gRNA modules. The final assembly will be validated by restriction mapping and then by complete sequencing. The final product will be provided as an assembled virus construct of 1 mg of transfection-grade CEP290-specific AAV shuttle plasmid DNA, free of endotoxins and sequence errors.
2. Preparation and analysis of AAV-H1-CRISPR stock. Packaging of the viral construct, purification of virus particles and molecular assessment of virus titer purity and infectivity will be performed by a cGMP-certified core facility, in accordance with industry standards (10). Production of a viral stock that is qualitatively and quantitatively suitable for preclinical testing. A cGMP-prepared and purified stock of sterile, endotoxin-free AAV, with a minimum infectivity of 0.9 IU/viral genome a minimal titer of 10¹² viral genomes/ml and a minimal yield of 10¹⁴ infectious units.
3. Quantitative characterization of AAV-H1-CRISPR genomic targets and off-target sites. Functional validation will be conducted using the immortalized retinal pigment epithelial cell line hTERT-RPE1, which harbors the LCA10 target sites. On-target and predicted off-target sites in the infected cell population will be deeply sequenced. The modified/unmodified allele ratio will provide a quantitative measure of efficiency; the on-target/off-target modification ratio will be a definitive measure of specificity. The viruses will be tested in vivo, using engineered mice and human biopsy samples. A quantitative analysis of target engagement. A virus that can create a deletion in >5% of CEP290 alleles in culture (a threshold of 5% is believed to lead to visual improvements in vivo), with <0.1% off-target effects.
1. Maguire A M et al. N Engl J Med. 2008; 358(21): 2240-2248.
2. Schimmer J et al. Hum Gene Ther Clin Dev. 2015; 26(4): 208-210.
3. Azvolinsky A. Nat Biotechnol. 2015; 33(7): 678-678.
4. Barrangou R. Science. 2014; 344(6185): 707-708.
5. Barrangou R et al. Expert Opin Biol Ther. 2015; 15(3): 311-314.
6. Shen B et al. Nat Methods. 2014; 11(4): 399-402.
7. Church G. Nature. 2015; 528(7580): S7.
8. Philippidis A. Hum Gene Ther Clin Dev. 2015; 26(2): 1-4. Summarized in a recent press report: http://www.reuters.com/article/us-sparktherapeutics-study-idUKKCN0RZ0WX20151005
9. Touchot N, Flume M. Nat Biotechnol. 2015; 33(9): 902-904.
10. Wright J F. Gene Ther. 2008; 15(11): 840-848.

### Example 3

Provided herein is an alternative approach to treating ADRP through the development of a CRISPR/Cas9 gene editing technology (Doudna, J. A. et al. Science 346, 1258096 (2014); Hsu, P D., et al. Cell 157, 1262-1278 (2014)), in which the RNA guided endonuclease is used in conjunction with customizable small guide RNAs (gRNAs) to target and cleave the mutant rhodopsin allele, which through error-prone non-homologous end joining (NHEJ) will specifically knock out expression of the mutant allele, without affecting the normal allele. Although this approach may result in expression of only 50% of the wild-type level of rhodopsin, animal data suggests that this should be sufficient to provide clinically useful rod function (Liang, Y. et al. The Journal of biological chemistry 279: 48189-48196 (2004)).

R135 mutations in rhodopsin result in the most aggressive and rapidly progressing form of retinitis pigmentosa (RP). Affected individuals, have night blindness during childhood with visual field losses before the second decade of life. Disease progression is unusually high, with an average 50% loss per year of baseline ERG amplitude and visual field area. By the fourth decade of life macular function is severely compromised (OMIM: http://www.omim.org/entry/180380).

By some estimates, R135 mutations account for the second most common rhodopsin mutations worldwide. The R135 mutations are particularly amenable to correction through NHEJ, as premature stop codons will likely result in degraded transcripts through non-sense mediated decay, relieving the dominant negative effect of this mutation. The most prevalent mutation, P347, occurs in exon 5 of rhodopsin, which presents additional challenges for correction by CRISPR genome-editing. Premature stop codons in the last exon of a gene are not susceptible to non-sense mediated decay.
1. Audo I et al. Invest Ophthalmol Vis Sci. (2010) July; 51(7):3687-700.
2. Pannarale M R et al. Ophthalmology. (1996) September; 103(9):1443-52.
3. Andréasson S et al. Ophthalmic Paediatr Genet. (1992) September; 13(3):145-53.

CRISPR Target Sites
WT sequence targeting:
hs086172474:
(SEQ ID NO: 111)
CCTGGCCATCGAGCGGTACGTGG
hs086172476:
(SEQ ID NO: 112)
GGCCATCGAGCGGTACGTGGTGG
hs086172479:
(SEQ ID NO: 113)
CCACGTACCGCTCGATGGCCAGG
hs086172480:
(SEQ ID NO: 114)
CACCACCACGTACCGCTCGATGG
R135G targeting:
hs086172474:
(SEQ ID NO: 115)
CCTGGCCATCGAGGGGTACGTGG
hs086172476:
(SEQ ID NO: 116)
GGCCATCGAGGGGTACGTGGTGG
hs086172479:
(SEQ ID NO: 117)
CCACGTACCCCTCGATGGCCAGG
hs086172480:
(SEQ ID NO: 118)
CACCACCACGTACCCCTCGATGG
R135W targeting:
hs086172474:
(SEQ ID NO: 119)
CCTGGCCATCGAGTGGTACGTGG
hs086172476:
(SEQ ID NO: 120)
GGCCATCGAGTGGTACGTGGTGG
hs086172479:
(SEQ ID NO: 121)
CCACGTACCACTCGATGGCCAGG
hs086172480:
(SEQ ID NO: 122)
CACCACCACGTACCACTCGATGG
R135L targeting:
hs086172474:
(SEQ ID NO: 123)
CCTGGCCATCGAGCTGTACGTGG
hs086172476:
(SEQ ID NO: 124)
GGCCATCGAGCTGTACGTGGTGG
hs086172479:
(SEQ ID NO: 125)
CCACGTACAGCTCGATGGCCAGG
hs086172480:
(SEQ ID NO: 126)
CACCACCACGTACAGCTCGATGG
gRNA sequences:
WT
hs086172474:
(SEQ ID NO: 127)
CCTGGCCATCGAGCGGTACGGTTTTAGAGCTAGAAATAGCAAGTTAAAA
TAAGGCTAGTCCGTTATCAACTTGAAAAAGTGGCACCGAGTCGGTGCTT
TT
hs086172476:
(SEQ ID NO: 128)
GGCCATCGAGCGGTACGTGGGTTTTAGAGCTAGAAATAGCAAGTTAAAA
TAAGGCTAGTCCGTTATCAACTTGAAAAAGTGGCACCGAGTCGGTGCTT
TT
hs086172479:
(SEQ ID NO: 129)
CCACGTACCGCTCGATGGCCGTTTTAGAGCTAGAAATAGCAAGTTAAAA
TAAGGCTAGTCCGTTATCAACTTGAAAAAGTGGCACCGAGTCGGTGCTT
TT
hs086172480:
(SEQ ID NO: 130)
CACCACCACGTACCGCTCGAGTTTTAGAGCTAGAAATAGCAAGTTAAAA
TAAGGCTAGTCCGTTATCAACTTGAAAAAGTGGCACCGAGTCGGTGCTT
TT
R135G
hs086172474:
(SEQ ID NO: 131)
CCTGGCCATCGAGGGGTACGGTTTTAGAGCTAGAAATAGCAAGTTAAA
ATAAGGCTAGTCCGTTATCAACTTGAAAAAGTGGCACCGAGTCGGTGC
TTTT
hs086172476:
(SEQ ID NO: 132)
GGCCATCGAGGGGTACGTGGGTTTTAGAGCTAGAAATAGCAAGTTAAA
ATAAGGCTAGTCCGTTATCAACTTGAAAAAGTGGCACCGAGTCGGTGC
TTTT
hs086172479:
(SEQ ID NO: 133)
CCACGTACCCCTCGATGGCCGTTTTAGAGCTAGAAATAGCAAGTTAAA
ATAAGGCTAGTCCGTTATCAACTTGAAAAAGTGGCACCGAGTCGGTGC
TTTT
hs086172480:
(SEQ ID NO: 134)
CACCACCACGTACCCCTCGAGTTTTAGAGCTAGAAATAGCAAGTTAAAA
TAAGGCTAGTCCGTTATCAACTTGAAAAAGTGGCACCGAGTCGGTGCTT
TT
R135W
hs086172474:
(SEQ ID NO: 135)
CCTGGCCATCGAGTGGTACGGTTTTAGAGCTAGAAATAGCAAGTTAAA
ATAAGGCTAGTCCGTTATCAACTTGAAAAAGTGGCACCGAGTCGGTGC
TTTT
hs086172476:
(SEQ ID NO: 136)
GGCCATCGAGTGGTACGTGGGTTTTAGAGCTAGAAATAGCAAGTTAAA
ATAAGGCTAGTCCGTTATCAACTTGAAAAAGTGGCACCGAGTCGGTGC
TTTT
hs086172479:
(SEQ ID NO: 137)
CCACGTACCACTCGATGGCCGTTTTAGAGCTAGAAATAGCAAGTTAAA
ATAAGGCTAGTCCGTTATCAACTTGAAAAAGTGGCACCGAGTCGGTGC
TTTT
hs086172480:
(SEQ ID NO: 138)
CACCACCACGTACCACTCGAGTTTTAGAGCTAGAAATAGCAAGTTAAAA
TAAGGCTAGTCCGTTATCAACTTGAAAAAGTGGCACCGAGTCGGTGCTT
TT
R135L
hs086172474:
(SEQ ID NO: 139)
CCTGGCCATCGAGCTGTACGGTTTTAGAGCTAGAAATAGCAAGTTAAA
ATAAGGCTAGTCCGTTATCAACTTGAAAAAGTGGCACCGAGTCGGTGC
TTTT
hs086172476:
(SEQ ID NO: 140)
GGCCATCGAGCTGTACGTGGGTTTTAGAGCTAGAAATAGCAAGTTAAA
ATAAGGCTAGTCCGTTATCAACTTGAAAAAGTGGCACCGAGTCGGTGC
TTTT
hs086172479:
(SEQ ID NO: 141)
CCACGTACAGCTCGATGGCCGTTTTAGAGCTAGAAATAGCAAGTTAAA
ATAAGGCTAGTCCGTTATCAACTTGAAAAAGTGGCACCGAGTCGGTGC
TTTT
hs086172480:
(SEQ ID NO: 142)
CACCACCACGTACAGCTCGAGTTTTAGAGCTAGAAATAGCAAGTTAAAA
TAAGGCTAGTCCGTTATCAACTTGAAAAAGTGGCACCGAGTCGGTGCTT
TT

### Example 4

Glaucoma, the leading cause of irreversible blindness worldwide (1), is an optic neuropathy in which progressive damage of retinal ganglion cell (RGC) axons at the lamina cribosa of the optic nerve head leads to axon degeneration and cell death (2). Currently, the only treatment, whether by eye drops, lasers or incisional surgery, is to lower intraocular pressure (IOP) and reduce the injury at the optic nerve head. Unfortunately, this is difficult in some patients while in others, the disease can continue to worsens despite aggressive IOP-lowering. The field has long needed an alternative therapeutic strategy that could complement IOP-lowering by mitigating the RGC response to residual axon injury. Moreover, the NEI has listed optic nerve regeneration amongst its Audacious Goals, and any regenerative therapy necessarily needs to tackle the issue of axotomized RGC survival. To this end, there is a great need to develop a neuroprotective that might directly interfere with the active genetic programs of RGC axon degeneration and/or axon injury-related cell death (3-6).

In order to identify, in a comprehensive and unbiased manner, genes that could serve as novel drug targets for neuroprotective glaucoma therapy, the entire mouse kinome (a druggable subset of the genome) was screened for kinases whose inhibition promotes RGC survival. For this screen, a high-throughput method was developed for knocking down gene expression in primary RGCs, from male and female C57Bl/6 mice (7), with small interfering RNA oligonucleotides (siRNAs) and coupled it with a quantitative assay of RGC survival (CellTiter-Glo, Promega) (5). Using this approach, an arrayed library of 1869 siRNAs was screened, targeting 623 kinases,

for the ability to increase the survival of RGCs three days after immunopanning (which necessarily axotomizes the cells). The top two hits were dual-leucine zipper kinase (DLK/MAP3K12) and its only known substrate, mitogen-activated protein kinase kinase 7 (MKK7). Using male and female floxed Dlk mice (8) and intravitreal injections of a Cre-expressing, capsid-modified (9, 10), adeno-associated virus (AAV2), it was shown that targeted disruption of Dlk rendered RGCs highly resistant to axon injury-induced cell death in the mouse optic nerve crush model (5). Moreover, since JUN N-terminal kinases (JNK) 1-3, typically activated by one or more upstream MAP3Ks, have been shown to play a central role in RGC cell death (11, 12), it was tested whether DLK (MAP3K12) was a relevant upstream trigger. Indeed, whereas wildtype mice intravitreally injected with AAV2-Cre responded to optic nerve injury with a robust upregulation of JNK signaling in the RGC soma and axon, floxed Dlk mice had a reduced activation of the pathway (5). Finally, using published kinase inhibitor profiling data (13, 14), the protein kinase inhibitor tozasertib was identified as an inhibitor of DLK and showed that it protected RGCs in both the rat optic nerve transection and glaucoma models (5). In some embodiments, target sequences for DLK/MAP3K12 may comprise a nucleotide sequence selected from the group consisting of SEQ ID NOs: 788-1023.

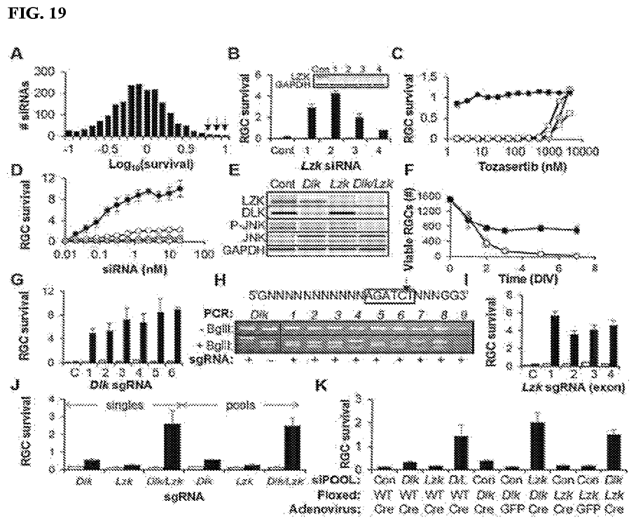

It was noticed that tozasertib consistently improved RGC survival in vitro more than DLK knockdown or knockout alone (5). This suggested that one or more additional kinase targets of tozasertib (of which there are more than 150) might cooperate with DLK to promote cell death and that simultaneous inhibition of both was to promote maximal RGC survival. In order to identify these other targets, the kinome screen was modified to include Dlk siRNA in every well and sensitize it to those library siRNAs that synergized with DLK knockdown to further increase RGC survival (FIG. 19A). The other modification was to allow 48 hours for extant protein turnover, before exacerbating the immunopanning injury with colchicine, a microtubule destabilizer that has been used to model axon injury in vitro and in vivo (15, 16). As expected, since Dlk siRNA was already present everywhere, none of the Dlk siRNA oligonucleotides from the library (which were the most efficacious in the initial kinome screen) improved survival above baseline. Instead, the top gene in this modified “synergy” protocol was a closely-related mixed lineage kinase, leucine zipper kinase (LZK/MAP3K13). Four additional Lzk siRNAs, with sequences not used in the initial screen, confirmed that LZK knockdown (FIG. 19B, inset) had little activity by itself, but was highly synergistic with DLK knockdown in promoting primary RGC survival (FIG. 1B). Moreover, this synergy was specific to LZK as none of the other members of the mixed lineage kinase family (i.e. MLK1-3) had any effect on survival (data not shown). Since tozasertib does inhibit LZK, in addition to DLK, (13, 14), it was considered whether LZK was indeed the “other” key target. Consistent with this hypothesis, individual knockdown of either LZK or DLK was partially neuroprotective and sensitized the cells to low doses of tozasertib (as one would expect if a key drug target was already genetically disrupted). Moreover,

combined knockdown of both DLK and LZK recapitulated the survival produced by the drug and further addition of tozasertib had no effect (as one would expect if all key drug targets were already genetically disrupted) (FIG. 19C). In some embodiments, target sequences for DLK/MAP3K13 may comprise a nucleotide sequences selected from the group consisting of SEQ ID NOs: 1024-1397.

While the evidence for LZK as a key mediator of RGC cell death was substantial, it was entirely based on individual siRNAs. It is important to note that the phenotype produced by any given siRNA is the biological sum of the canonical “on-target” silencing mediated by all 21 nucleotides and the promiscuous “off-target” silencing mediated by a six-to-seven nucleotide seed sequence (17, 18). The latter can silence hundreds of targets, unfortunately causing it to dominate phenotypes and siRNA screen results (19). Thus, two complementary approaches were chosen to validate the LZK finding, siPOOLs and clustered regularly interspaced short palindromic repeats (CRISPR)-mediated gene knockouts. siPOOLs are pools of 30 siRNAs targeting a common gene. Because each of the 30 component siRNAs has a different set of off-targets but shares a common on-target, the siPOOL provides a 30-fold enrichment for on-target versus off-target silencing (20). As expected, while the Lzk siPOOL only minimally improved primary RGC survival, the combination of Dlk and Lzk siPOOLs was highly synergistic in terms of both survival (FIG. 19D) and suppression of JNK signaling (FIG. 19E). The phenotype was not an artifact of the

CellTiter-Glo assay as similar results were obtained with conventional stains of viability (FIG. 19F). For the knockout approach with CRISPRs, the fact that the primary RGC platform was built around the delivery of small RNA (i.e. siRNAs) was leveraged. By isolating RGCs from mice expressing S. pyogenes Cas9 (spCas9) at the Rosa26 locus (21) and instead transfecting with in vitro-transcribed synthetic guide RNAs (sgRNAs), Cas9 could be targeted to cut in a given target gene. The cells then repair this cut site with nonhomologous end joining, giving rise to small (˜1-20 bp) insertion/deletions (indels) which can frameshift the downstream protein. To validate the approach, a series of sgRNAs against Dlk were synthesized and showed that they synergized with Lzk siPOOLs to increase survival (FIG. 19G) and that they produced target-specific indels with no detectable off-target effects (FIG. 19H). The reciprocal approach, i.e. Dlk siPOOLs and Lzk sgRNA, produced a similar result (FIG. 191) as did the use of only sgRNAs, i.e. Dlk sgRNA and Lzk sgRNA (FIG. 19J). The floxed Lzk mouse were recently generated and validated the knockout in vitro (FIG. 19K). Homozygous Dlkfl/flLzkfl/fl mice were bred and tested to see if combined targeted disruption of Lzk and Dlk in vivo leads to enhanced RGC survival and JNK pathway suppression, following optic nerve injury. At least in primary RGCs, however, it is clear that LZK can mediate injury signaling when DLK is nonfunctional, requiring simultaneous inhibition of both DLK and LZK for maximal neuroprotection.

It was next sought to identify non-kinase members of the injury signaling pathway and thus we expanded our platform to screen a whole-genome library of 16,877 siRNA minipools (four per gene). In order to improve the signal-to-noise of the assay and facilitate screening at the whole-genome scale, the fact that LZK inhibition does little to improve baseline survival but highly sensitizes cells to further DLK inhibition was leveraged. Thus, in addition to the standard library siRNA minipool, each well also received the Lzk siPOOL. To analyze the results, the fact that genome-scale screens sample siRNAs with every possible seed combination multiple times was taken advantaged, thus permitting two distinct bioinformatics approaches to address pervasive off-target effects. First, the survival effect of a given seed can be tracked as it appears dozens of times throughout the library. This can then be used to generate a correction factor which helps to subtract out the off-target contribution to the phenotype and reveal the component mediated by the on-target (19). Secondly, in addition to knowing the survival/toxicity produced by each seed, one can also predict all its possible off-targets. Then, using a Haystack analysis to search for commonly-targeted genes by similarly-behaving seeds, one can actually try to deconvolute the off-target effects and identify the genes whose off-target silencing affects survival (22).

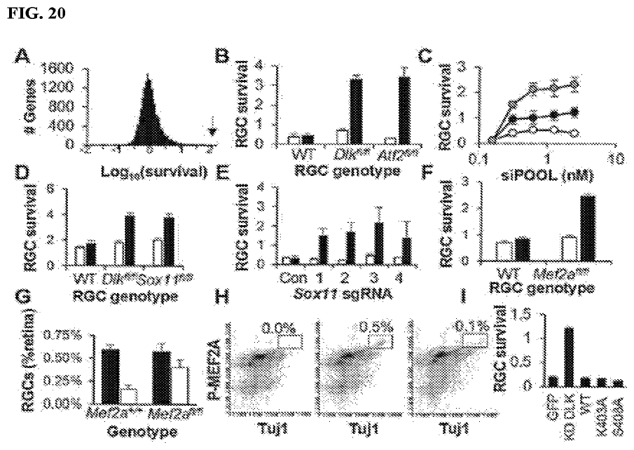

When the results using the first method (seed-corrected, on-target) were analyzed, it was found that DLK was the top gene amongst all 16,877 genes (FIG. 20A). Moreover, MKK4, MKK7, JNK1, JNK2, JNK3 all ranked in the top 1% (data not shown). The screen also identified two known JNK-dependent transcription factors, JUN and ATF2. The former had been previously validated as a key mediator of RGC cell death in a rodent model of optic nerve injury (23). To test the role of the latter, primary RGCs were isolated from wildtype versus floxed Atf2 mice (24) and transduced them with Cre- versus GFP-expressing adenovirus. As expected, targeted deletion of Atf2 increased survival, comparable to that produced by Dlk deletion (FIG. 20B). Together, these results provided confidence that the screen was able to identify known DLK/JNK pathway members. Next it was attempted to validate the novel genes, which had no prior association with DLK or JNK. The top hit, using the Haystack analysis, was the sex determining region Y (SRY)-box 11 (SOX11) transcription factor. Along with SOX4, SOX11 had been previously shown to be critical for RGC survival and differentiation during development (25). In order to validate that SOX11 played a paradoxical role in RGC cell death, the Sox11 siPOOL was first tested and found that it indeed cooperated with either Lzk (FIG. 20C) or Dlk (data not shown) siPOOLs to increase primary RGC survival. Two knockout models: transducing Sox11fl/fl RGCs (26) with Cre- versus GFP-expressing adenovirus (FIG. 20D) were examined and transfected with spCas9-knockin RGCs with sgRNAs targeting the single Sox11 exon (FIG. 20E). In both cases, targeted disruption of Sox11 improved RGC survival. The fact that this developmental survival gene could play a role in injury signaling in the adult is supported by microarray studies which have shown that SOX11 is amongst the most highly upregulated genes in response to optic nerve crush and in a manner that is nearly entirely DLK-dependent (6). The Sox11fl/fl mouse is currently being used to validate the survival phenotype in vivo. To ensure that potential RGC cell death pathway members were not missing because the screen was overly-sensitized to respond to DLK pathway inhibition, a different whole-genome siRNA library was screened in the absence of the Lzk siPOOL. Primary RGCs were transfected with an arrayed library of 52,725 siRNAs, targeting 17,575 genes. Again, the top hits included known genes like DLK, JUN, ATF2, MKK4 and MKK7. However, this time we also identified another transcription factor, myocyte enhancer factor 2A (MEF2A), which has a prominent role in neuronal survival and differentiation (27-29). As with other candidates, MEF2A was validated with an siPOOL (data not shown), sgRNAs (data not shown) and by isolated RGCs from conditional knockout (Mef2afl/f) mice (30) and transducing with adeno-Cre versus -GFP (FIG. 20F). Moreover, knockout of MEF2A in vivo was demonstrated to protect RGCs in the mouse optic nerve crush model (FIG. 20G). Finally, using Dlkfl/fl mice, it was shown that MEF2A is phosphorylated in response to axon injury, in a DLK-dependent manner (FIG. 20H).

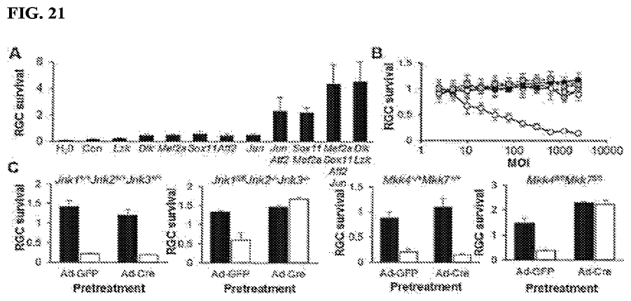

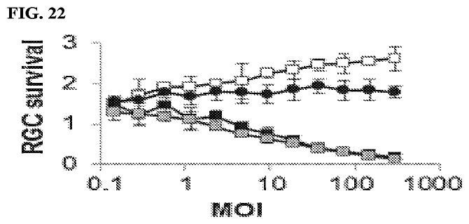

By this point, four different transcription factors (i.e. ATF2, JUN, SOX11, MEF2A) were identified, each which had a partially protective effect when disrupted. Given the lesson of redundancy from DLK and LZK, it was considered the possibility that these four transcription factors might need to be simultaneously inhibited for maximal neuroprotection. Thus, wildtype RGCs were transfected with Mef2a, Jun, Sox11 or Atf2 siPOOLs, alone or in combination, and survival was measured 96 hours later. The results showed that inhibition of the four transcription factors is highly synergistic, increasing survival as much as simultaneous DLK/LZK inhibition (FIG. 21A). To determine if these four transcription factors were genetically downstream of DLK, wildtype RGCs were transfected with Dlk/Lzk siPOOLs, with or without the siPOOLs targeting the four transcription factors. After two days, DLK signaling was reconstituted with adenovirus (expressing mouse siRNAinsensitive, rat DLK) and survival was measured an additional two days later. Whereas 80-90% of RGCs were killed by DLK overexpression in the presence of the four transcription factors, simultaneous knockdown of MEF2A, SOX11, JUN and ATF2 completely abolished cell death (FIG. 21B). Finally, using a combination of JNK1-3 and MKK4/7 knockouts and DLK (data not shown) or LZK overexpression (FIG. 21C), it was demonstrated that DLK/LZK have no toxic effects in the absence of an intact MKK/JNK pathway. Taken together, the functional genomic screening can be summarized by a model in which axon injury triggers a kinase signaling cascade of DLK/LZK, MKK4/7 and JNK1-3, leading to the activation of four transcription factors, MEF2A, SOX11, JUN and ATF2, and ultimately cell death.

Targeting DLK as a neuroprotective strategy for glaucoma has several attractive features. As mentioned above, it has a robust, evolutionarily-conserved phenotype (5, 31), it was identified using an unbiased method and it is readily druggable with small molecule inhibitors. In addition, it has been validated

independently and DLK inhibition, rather than simply delaying cell death, seems to prevent it altogether (6). Moreover, the cells kept alive with DLK inhibition remain electrophysiologically active in vitro (5) and, in vivo, retain a relatively healthy gene expression pattern, despite their previous injury (6). Finally, some (4, 6, 8) but not all (32) data suggest that DLK plays a role in the axon degeneration program triggered by axon injury. On the other hand, DLK inhibition retards axon regeneration (6), a potential limitation with respect to the generation of neuroregenerative strategies (33-35). This underscores the need to dissect out the pathway members responsible for cell death decisions from those that are responsible for axon regeneration. Furthermore, despite their central role in cell death (and regeneration), the mechanisms by which DLK and LZK are activated in response to axonal injury and by which they activate the downstream transcription factors has yet to be elucidated.

As disclosed herein, functional genomic screening was integrated with a CRISPR/AAV therapeutic platform in order to further probe the DLK/LZK cell death pathway and seamlessly interdict the activity of the various candidate neuroprotective targets with a viable gene therapy vector.

In some embodiments, an enhanced set of RNA-based screening paradigms, using sgRNAs and networks of siRNAs, will be used in order to identify novel DLK/LZK pathway members, including the as-yet-unidentified upstream activator(s) and targets that might dissociate cell death and regeneration. In some embodiments, a CRISPR/AAV vector will be developed that allows for rapid validation of hits from SA1 in rodent models of optic neuropathy. Variants will be created of these neuroprotective viruses which are suitable for gene therapy applications, thereby expanding the space of potential targets to include non-druggable gene products and even networks of genes. In some embodiments, a combination of proteomics and loss-of-function/gain-of-function experiments will be used to probe the mechanism by which DLK regulates the downstream mediator, MEF2A and determine if MEF2A inhibition can serve as a therapeutic strategy to promote RGC survival without preventing regeneration.

From a mechanism standpoint, the screening platform uniquely combines a disease-relevant primary neuron (i.e. RGCs) with arrayed, whole genome-scale, RNA-based screens that can be completed in a few weeks, to give us an unbiased and comprehensive tool to better understand RGC cell death signaling. Indeed, SOX11, critical for RGC development, is a perfect example of how such an approach can make novel and unexpected discoveries about genes involved in cell death. The modifications proposed below (i.e. to screen gene networks and to use CRISPR technology) will open up the space of potential neuroprotective targets that can be identified.

From a therapeutic standpoint, CRISPR editing in RGCs will be utilized to develop AAV/CRISPR therapeutics in vivo. In some embodiments, a, AAV/CRISPR packaging of two separate cistrons (i.e. one expressing the gRNA and the other expressing spCas9) into a single AAV particle, overcomes a major limitation in the use of CRISPRs for gene therapy. This creates the potential for a gene therapy-based approach to both validate hits from our screens with dramatically improved throughput and to be able to target these pathway members therapeutically, including those that would not be readily druggable with small molecules. Overall, both the DLK target pathway itself and the approaches constitute an innovative strategy for the development of novel neuroprotective therapies for glaucoma and other forms of optic nerve disease.

Use enhanced RNA-based screens in primary RGCs to identify the novel DLK/LZK pathway members, including the as-yet-unidentified upstream activators.

The mechanism by which DLK and LZK are activated in response to optic nerve injury remains to be determined. In D. melanogaster and C. elegans, an E3 ligase (called highwire and RPM-1, respectively), suppresses the basal protein levels of the DLK homolog (called Wallenda and DLK-1, respectively). In response

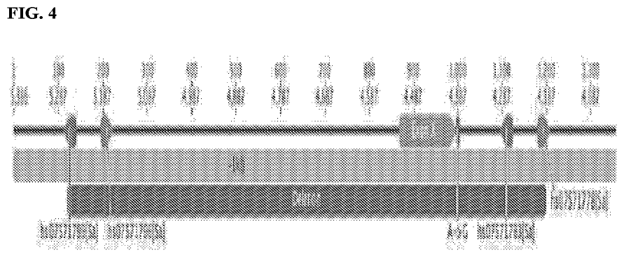

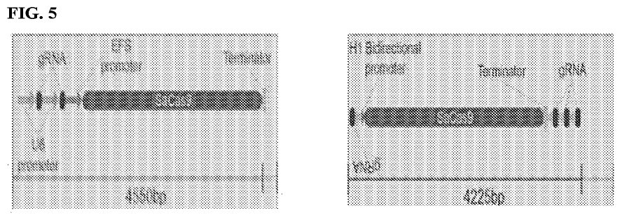

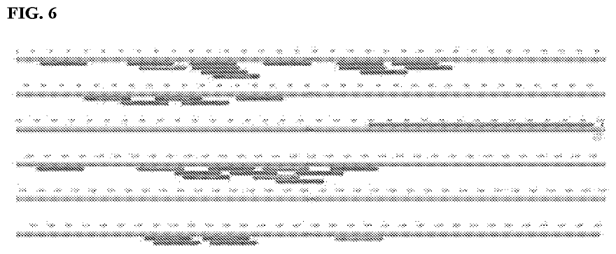

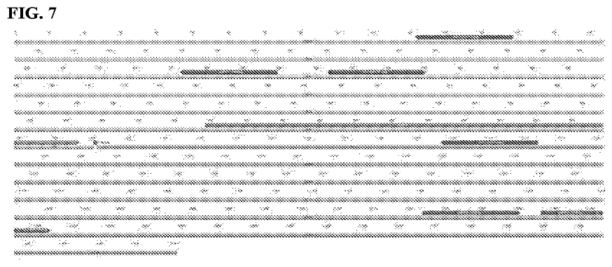

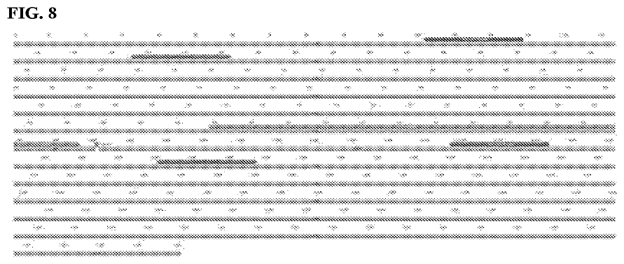

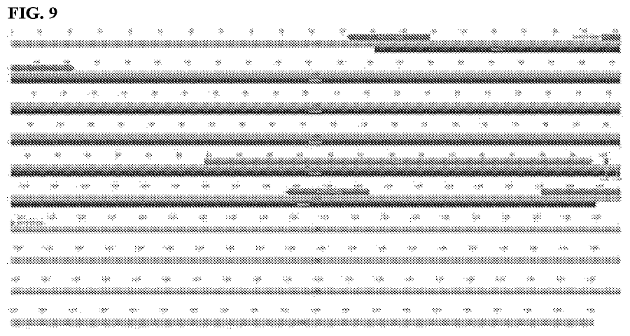

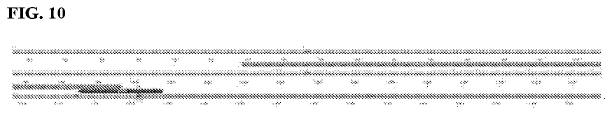

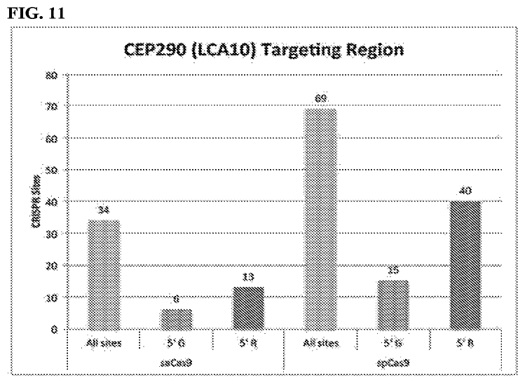

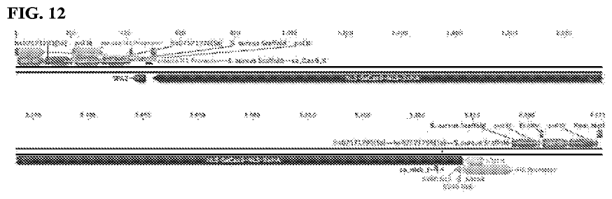

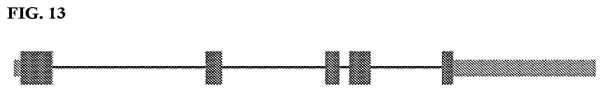

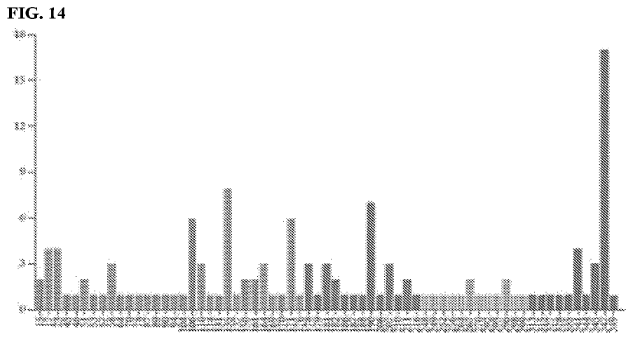

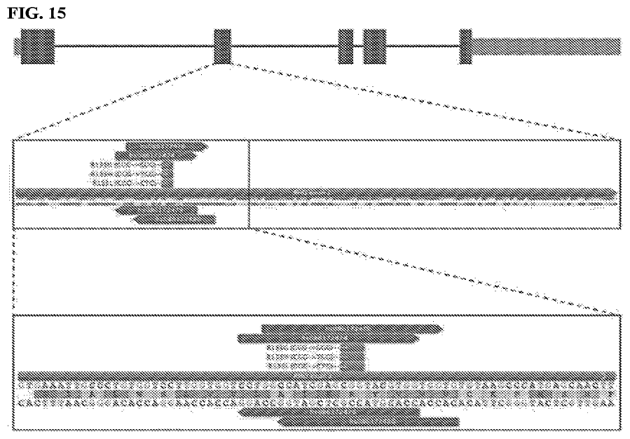

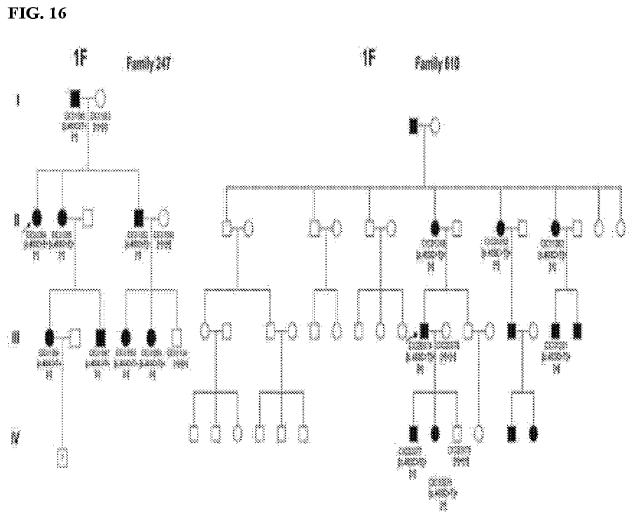

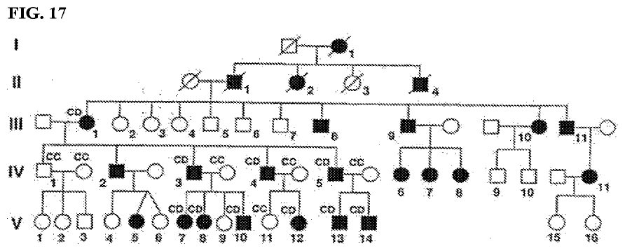

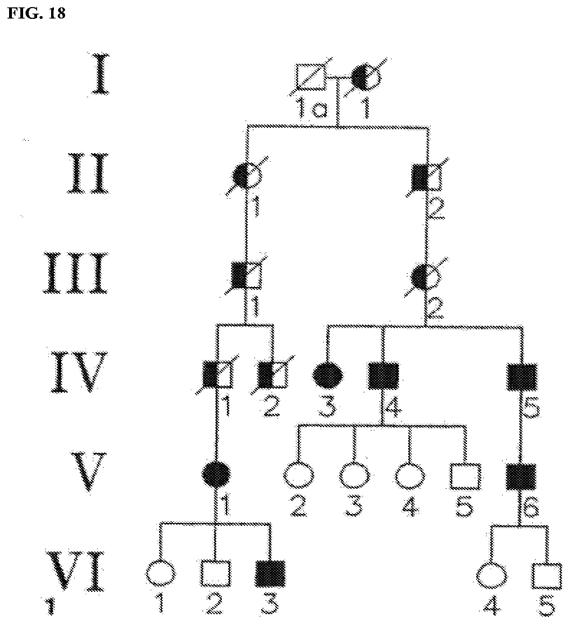

to axon injury, this suppression is relaxed, leading to DLK accumulation and cell death. Interestingly, the role for the Highwire/RPM-1 homolog in mouse, PHR1, is less clear. As would be expected, disruption of Phr1 in dorsal root ganglion cells increases the level of DLK (36, 37), but in RGCs, knockdown of PHR1 has no effect on DLK levels or survival (data not shown) and the conditional Phr1 knockout mouse has normal RGC survival (38, 39). The intrinsic calcium-sensing motif described in C. elegans DLK-1 (40) does not appear to be functionally conserved as replacement of endogenous mouse LZK (the only mouse DLK-1 homolog which contains the motif) with either wildtype human LZK or a mutant lacking the hexapeptide motif leads to equivalent cell death (FIG. 4). Finally, in vertebrates, there are several DLK phosphorylation sites, including S643, S302, S295, S302, T306 and S643 which do not appear to be regulated by the known DLK kinases (i.e. JNK and DLK), suggesting the possibility of novel, upstream regulatory kinases (37). While the kinome and genome screens described above were sensitized to look for DLK pathway members, any genes upstream of DLK/LZK have not been identified. Of note, these screens had two inherent limitations. First, knockdown rather than knockout, may be insufficient to yield a phenotype for some genes. Supporting this idea, it has been shown that knockdown-based screens can yield distinct hits when directly compared to knockout-based screens (41). Second, as was the case with DLK/LZK, MKK4/7, JNK1/2/3 and SOX11/ATF2/JUN/MEF2A, multiple members of a given tier may need to be simultaneously inhibited in order to produce a robust phenotype. To address these potential limitations, two key changes to our screening strategy will be made. For our initial rescreens, the kinome will be focused on given its relatively small size, bioactivity and druggability.
1. Screen an Arrayed sgRNA Library Targeting the Mouse Kinome to Identify Genes Whose Targeted Disruption Improves RGC Survival.

To address the first limitation mentioned above, CRISPR-based screening will be performed, somewhat analogous to the iCRISPR system (42). A colony of spCas9-expressing mice was scaled and robust survival was demonstrated when we isolate RGCs from these mice and transfect them with sgRNAs instead of siRNAs (FIG. 19G-J). Indeed, the Z′ was between 0.3-0.5, with 0.5, indicating that cells in a screen transfected with positive control sgRNA (i.e. targeting Dlk) have a survival that is more than 12 standard deviations higher than cells transfected with a control tracrRNA (which is missing the 20 nucleotide protospacer sequence that directs it to a particular gene). As shown in FIGS. 19G and J, the variability amongst different sgRNAs targeting the same gene is rather small and that three sgRNAs per gene should be sufficient coverage. As was done with DLK and LZK, target sites will be selected which have the sequence GN₁₉NGG in order to be compatible with T7-transcription and the spCas9, protospacer-adjacent motif (PAM). Priority will be given to those sites that are bioinformatically predicted to have greater on-target and lower off-target cutting (43-45). Moreover, target sites will be selected in the 5′ half of the gene and, when possible, in critical domains. The former ensures that frameshift mutations produced by indels affect more than half the remaining protein while the latter allows the one-third of indels which stay in frame to still disrupt protein function. To allow for a direct comparison to past work, the library will target the same 623 kinases present in the Sigma kinome siRNA library. For high-throughput synthesis, the template for each sgRNA's transcription is a gene-specific 60-mer oligonucleotide annealed to a common 80-mer that contains the tracrRNA sequence (46).

For the screen, RGCs will be immunopanned from spCas9-knockin mice and seeded at 1,000 cells per well in 384-well plates. The library of sgRNAs will be reverse transfected with NeuroMag (Oz Biosciences) at 30 ng per well, in duplicates. Since 1 nM Lzk siPOOL worked well to sensitize a whole-genome siRNA library to DLK inhibition, the same reagent will be added to all wells of this screen. Each plate will contain negative (tracrRNA) and positive (Dlk) control sgRNAs that serve as quality controls and normalization standards (to allow for plate-to-plate comparisons). After 48 hours, each well will be treated with 1 μM colchicine to promote DLK/LKZàJNK signaling and lower background survival (15, 47). Finally, after an

additional 48 hours, survival will be measured using CellTiter-Glo and normalized to the median negative control wells on each plate. Genes will be scored and ranked based on the mean and median activity for the three sgRNAs. Active sgRNAs will be retested in spCas9 and wildtype RGCs to ensure that the activity is spCas9-dependent. As with siRNAs, secondary screening will be performed by synthesizing new sgRNAs with sequences not tested in the initial screen.

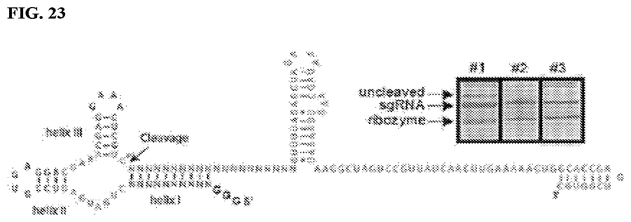

Given that the platform of transfecting primary RGCs with RNA and measuring survival has been extensively tested, any issues with the screen itself are not anticipated. Moreover, given the results from the whole-genome siRNA screen which used Lzk siPOOL to sensitize the system to DLK inhibition and yielded the Dlk siRNA minipool as the top hit amongst 16,877 genes, the strategy will be able to identify DLK pathway members. The main issue will be the labor involved in synthesizing the sgRNA library. Pooling templates and in vitro transcribing pools of three sgRNAs are further contemplated. Multiple sgRNAs targeting the same gene should lessen the chance that a given locus escapes targeting by having all the indels fortuitously end up as a multiple-of-three nucleotides. However, at least with LZK and DLK, increased efficacy with the pooling strategy have not been observed (FIG. 19J), possibly because of the already high indel frequency (FIG. 19H). To circumvent some smaller kinases having too few acceptable spCas9 targets, siTOOLS (Munich, Germany) will be utilized to test sgRNAs with a 5′ fusion to a hammerhead ribozyme (FIG. 23) thereby eliminating the need for the sgRNA to begin with a guanine (since T7 transcription would start with the ribozyme) and increasing the number of targets by a factor of four. It has been shown that this system produces the correct cleavage products and are currently testing the efficacy of sgRNAs made in this fashion (FIG. 23). Finally, mouse sgRNA libraries are now commercially available from Dharmacon if needed. Dharmacon, involving the co-transfection of a gene-specific crRNA and a common tracrRNA, had only 50% of the activity of the chimeric sgRNAs used above (data not shown). If successful, the next step would be to design and screen whole-genome sgRNA libraries.

2. Screen an siRNA Library, Grouped to Target Kinase Networks, in Order to Identify Genes Whose Knockdown Improves RGC Survival.

Even if a kinase plays a key role in RGC cell death, its phenotype when inhibited could be masked by redundant/compensatory kinases. The sensitized screens were one approach to circumvent this limitation, but relied on already knowing one member of the redundant pair (e.g. LZK in the genome-wide screen). As an alternative, more unbiased approach, an siRNA library can be screened in which every well simultaneously targets multiple kinases, grouped because they may be part of a redundant signaling network. The Sigma kinome siRNA library will be initially utilized, condensed into 623 minipools (three siRNAs per gene), and then combining the minipools in novel combinations. As it is not feasible to try all pairwise kinase combinations (6232), three distinct bioinformatics approaches will be utilized to

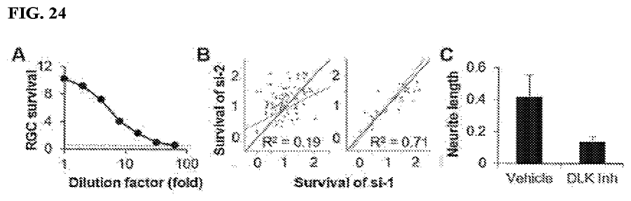

create the pools-of-minipools. First, and conceptually the simplest, siRNAs targeting kinases with the highest degrees of homology (e.g. JNK1, 2 and 3) will be grouped together. Based on the ENSEMBL database, 488 genes in the Sigma kinome siRNA library shared significant homology with another member, leading to 1626 pairs (as a given gene could be homologous to more than one other member). To make the numbers more manageable, these pairs were further clustered into 111 groups with a median of four members per group. Second, kinases with overlapping spectra of substrates might be redundant. Phosphorylation reactions with 289 human kinases were performed on protein microarrays composed of 4,191 unique, full-length human proteins and identified 3,656 kinase-substrate relationships (48). Of the 198 genes in the Sigma kinome siRNA library which were included in the analysis, there were 2,089 pairs with significant overlap of their substrate spectra. As kinases can have overlapping spectra with more than one other kinase, this led to 3,386 potential groups with a median of 11 genes per group. Finally, the fact that all 121 kinases, and all of their pairwise combinations, in S. cerevisiae have been assayed for genetic interactions in order to produce a comprehensive epistasis map (E-MAP) was taken advantage (49). It was reasoned that some of these pairwise genetic interactions might be conserved in mouse and, in some cases, indicate redundant/compensatory pathway members (the genetic substrate of the synthetic lethality interaction). 1,674 pairwise kinase interactions were identified amongst the 223 kinases in the Sigma library which have a yeast homolog, giving rise to 1,003 groups with a median size of two genes. In total, 4,500 siRNAs groups, with the largest group containing 19 kinases. Finally, a pilot experiment was performed to ensure that the pools with the most targets (e.g. 19 kinases), most of which are likely to be inactive, would not dilute out an active pair. It was reassuring to note that RGCs transfected with Dlk and Lzk siRNA minipools produced a detectable phenotype even when diluted 64-fold with an inactive siRNA (FIG. 24A). The screen itself will be done in duplicate, with each plate containing negative (Lzk/Lzk) and positive controls (Dlk/Lzk) for synergy and as normalization standards. The survival data will be adjusted with the seed-correction factors that we have already generated and mined to look for combinations which appear in multiple survival-promoting wells. These pools will then be deconvoluted to identify the active mediators. Finally, secondary confirmation will involve the use of independent siRNAs with sequences not tested in the Sigma library.

We have confirmed that our three approaches to generating kinase combinations yielded lists that included at least one known interacting kinases (e.g. phospho-networks—INK1/2/3 and MKK4/7; homologs—JNK1/2/3, MKK4/7, and DLK/LZK; yeast E-MAP—DLK/LZK). Also, because screens can be done in a matter of weeks, the process can be iterative, with the results of the screen leading to modifications in the grouping algorithms. The actual construction of the library should be relatively easy given access to a Labcyte Echo 550 acoustic liquid

handler which can assemble all 4,500 groups in a matter of hours, without the use of pipet tips. One obstacle may be the combination of so many siRNAs in a single well could lead to numerous off-target interactions. As a complementary or alternative strategy, siTOOLS (Munich, Germany) may be utilized to build a mouse kinome library using siPOOLs. This would have the advantage of enriching for on-target effects by a factor of 30. Indeed, when A439 cells were transfected with siRNAs versus siPOOLs targeting kinases and assayed for survival, siRNAs targeting the same gene had an R2 of only 0.19 (FIG. 24B). In contrast, independent siPOOLs targeting the same gene had an R2 of 0.71.
3. Determine if Validated Hits from Section 1 or 2 Above Affect DLK/LZK Signaling.

As was done with DLK and LZK (FIG. 19E), RGCs will be transfected with siRNAs, siPOOLs or sgRNAs (from Section 1 and 2 above) targeting validated kinases, alone or in combination with Lzk siPOOL (to sensitize the cells to pathway inhibition) and immunoblotted for phosphorylated MKK4, MKK7 (the direct substrates of DLK/LZK), INK and JUN at 24-48 hours. Kinases whose knockdown or knockout suppresses JNK pathway signaling will be considered potential

upstream activators of the DLK/LZK. Those that promote survival without affecting MKK/JNK phosphorylation must be reducing the sensitivity to DLK, as it is sufficient to trigger RGC cell death.

While the effects of siRNA are easy to detect on unselected, whole populations, sgRNAs are more difficult because knockouts do not occur in 100% of the cells. To address this issue, RGCs will be transfected with the sgRNAs and subjected to the standard 96-hour protocol with colchicine in order to provide a strong selection pressure and enrich for knockout cells. Then, depending on the number of cells remaining, JNK pathway status will be measured by immunoblotting for JNK phosphorylation or immunofluorescence using antibodies against phosphorylated-JUN.

4. Determine if Validated Hits from Section 1 or 2 Genetically Function Upstream or Downstream of DLK and LZK.

RGCs will be transfected as in Section 3 and then, after sufficient time for selection, the remaining cells will be challenged with adenovirus overexpressing wildtype rat DLK or human LZK. Genes that are upstream of DLK/LZK should have their knockdown/knockout phenotype reversed with DLK/LZK overexpression. As a complementary approach, adenoviruses will be produced overexpressing the cDNA for the candidate kinases (including any known mutants which render the kinase constitutively active) and tested whether their overexpression promotes RGC cell death and whether it can be blocked with DLK/LZK inhibitors and/or Dlk/Lzk siRNA (as would be expected if they function upstream of DLK/LZK).

Reversing the survival phenotype with our readily-available, wildtype rat DLKexpressing adenovirus and not a true constitutively active mutant may be performed. If the survival phenotype can be reversed, a DLK mutant (S584A/T659A) will be subcloned and expressed which is thought to have increased activity (50). Finally, although the main purpose of Section 1 above is to identify upstream activators of DLK and LZK, kinases will be pursued that appear to be functioning downstream/independent of DLK/LZK.

5. Determine if Validated Hits from Section 1 or 2 Promotes Survival without Affecting Axon Regeneration.

RGCs will be transfected with siRNAs, siPOOLs or sgRNAs (from Section 1 and 2) targeting validated kinases, alone or in combination with Lzk siPOOL (to sensitize the cells to pathway inhibition) and, after 72 hours, stained with calcein AM. A Cellomics automated microscope will be used to image the cells and calculate the overall neurite length. Ideally, genes will be identified whose inhibition promotes survival without affecting neurite outgrowth.

To confirm that the neurite-outgrowth assay might be predictive of regeneration in vivo, we have previously shown that primary RGCs treated with DLK/LZK inhibitors and/or siRNAs have dramatically-reduced neurite length compared to control-treated cells (FIG. 24C). Moreover, complementary experiments in Section 2 may be used to validate the in vivo relevance of our findings.

6. Develop a CRISPR System, Compatible with AAV Delivery, in Order to Knockout Target Gene Function in RGCs In Vivo.

The screening work has previously identified multiple europrotective targets in RGCs and the approaches outlined in Sections 1 and 2 will further expand the list of candidates. Additional steps include: the validation of these hits in vivo in rodent models of optic neuropathy, the prioritization of the validated hits, and the subsequent development of specific inhibitors of the prioritized targets. A potentially ideal approach would be a gene therapy-based strategy that could both quickly validate hits (i.e. knockout genes in vivo without having to create/obtain knockout mice) and later serve as the therapeutic itself. As demonstrated in FIG. 19G-J, CRISPR-based systems can be used in RGCs to robustly disrupt gene function in vitro. The CRISPR technology will be used for in vivo genome editing of RGCs, taking advantage of the ability of AAV2 to effectively transduce RGCs (9, 10). Unfortunately, AAV capsids have a packaging capacity of ˜4.8 kb that has prevented the delivery of both the spCas9-expression (˜4.8 kb) and gRNAexpression cassettes (˜0.4 kb) in a single virus. AAV/CRISPR systems have been used in vivo but have had to rely on either: 1) the use of two different AAVs (51), which is suboptimal for gene therapy, or; 2) the use of the smaller S. aureus Cas9 (saCas9) (52), which has a more restrictive PAM and reduces the number of potential target sites by a factor of four. This problem is compounded by the fact that the gRNAs are typically expressed off the U6 promoter, which initiates at a guanine, limiting target sites to GN₁₉NGG (spCas9) or GN19NNGRRT (saCas9). Recently, it has been showed that the H1 promoter, which can initiate at either adenine or guanine, doubles the number of potential Cas9-targets (53). Naturally, the H1 promoter drives the transcription of its canonical Pol III transcript, H1RNA, in one direction, and, in the other direction, a ubiquitously-expressed Pol II transcript called Parp2 (54, 55) (FIG. 28A). This compact (˜0.2 kb), bidirectional H1 architecture allows spCas9 and gRNA to be expressed from a single promoter and packaged into a single AAV vector.

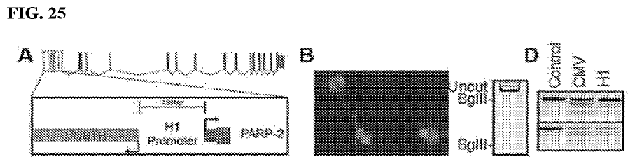

To test this system, an AAV construct was generated that used the H1 promoter to express an mCherry-histone 2B fusion in the Pol II direction and the Dlk #4 gRNA (5′GNNNNNNNNNNNAGATCTNNNGG 3′ (SEQ ID NO: 163)), which conveniently targets a BglII site (FIGS. 19G and 1H), in the Pol III direction (Dlk gRNA:H1:H2B-mCherry). Because the total size is under 2.4 kb, one of the inverted terminal repeats was mutated to generate self-complementary (sc) AAV, known to increase and expedite gene expression (56). Viral particles were produced and used to transduce primary RGCs isolated from the spCas9-P2A-GFP knockin mice. After just five days in the presence of tozasertib (to prevent cell death and avoid selection), robust reporter expression with nearly 100% transduction (FIG. 25B, left) and greater than 90% loss of the BglII site, i.e. gene editing (FIG. 25B, right), was observed. Separately, NIH3T3 cells were transfected with a plasmid (non-viral) that expresses both spCas9 and gRNA from a single H1 promoter and showed that it leads to robust cutting, comparable to the traditional two vector system (FIG. 25C). Together, these studies validate the scAAV system (to be utilized in Sections 8, 9, and 15) and the Pol II/III-competence of the bidirectional H1 promoter. As described below, a single AAV delivery system was generated that expressed both spCas9 and gRNA. In some embodiments, sequences for self-complementary (sc) AAV may comprise a nucleotide sequence selected from the group consisting of SEQ ID NOs: 1398-1400.

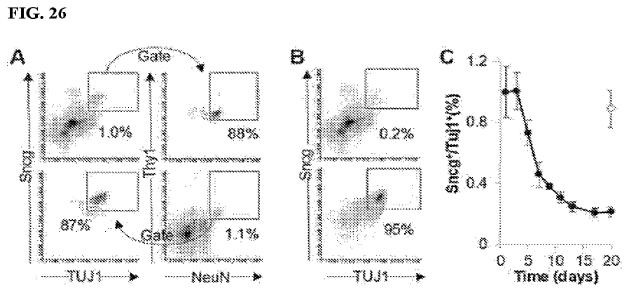

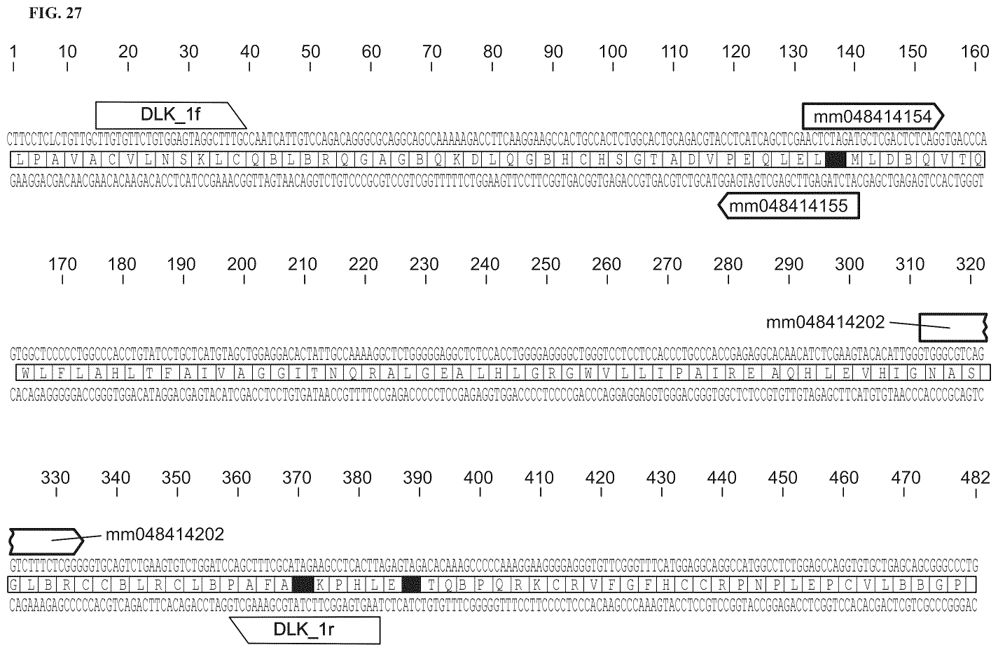

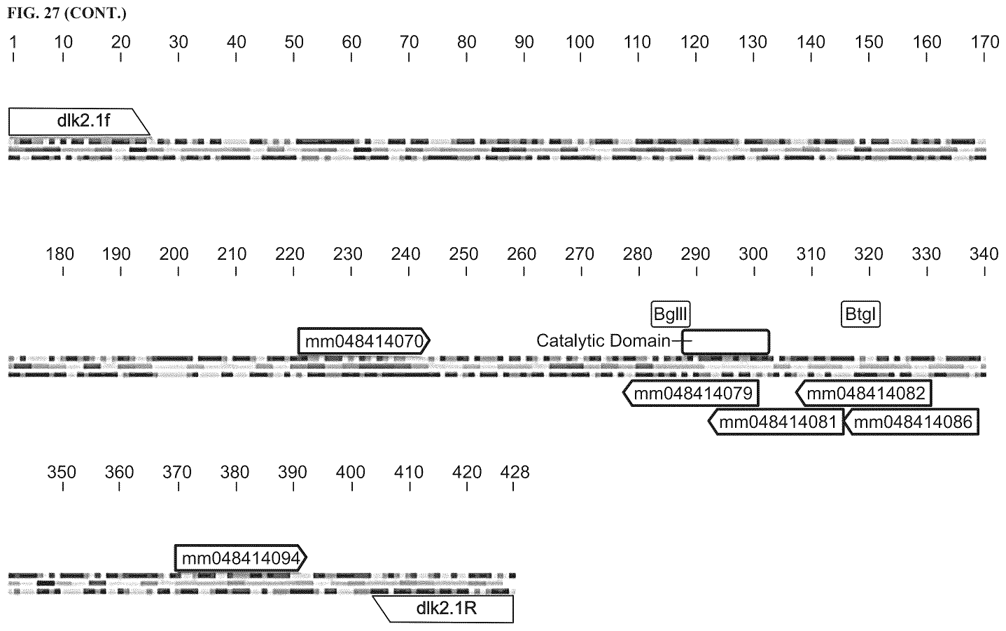

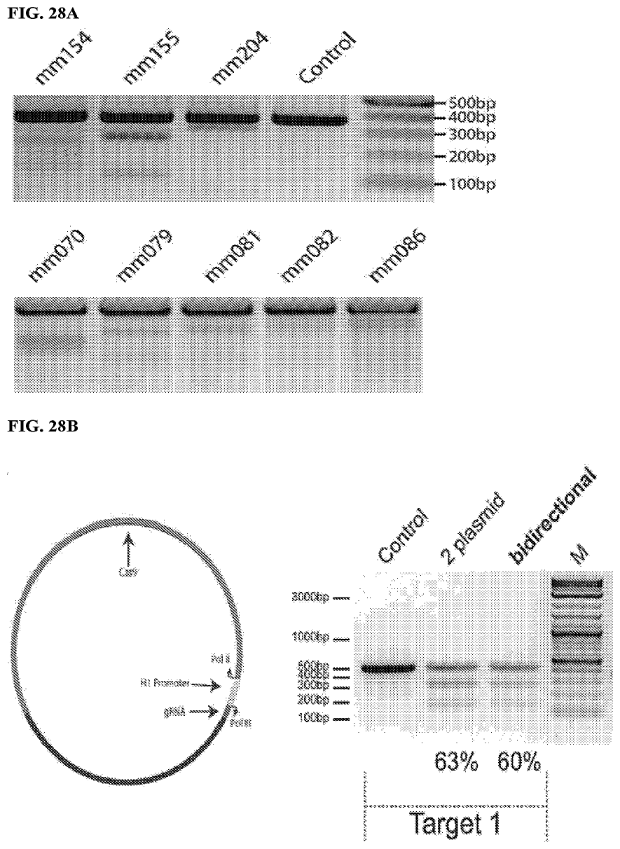

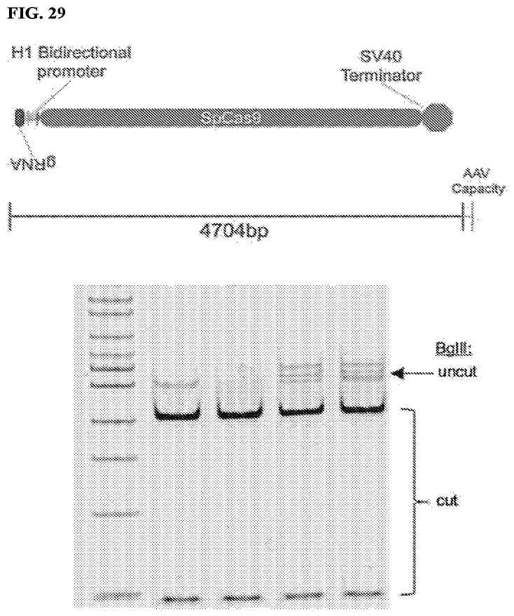

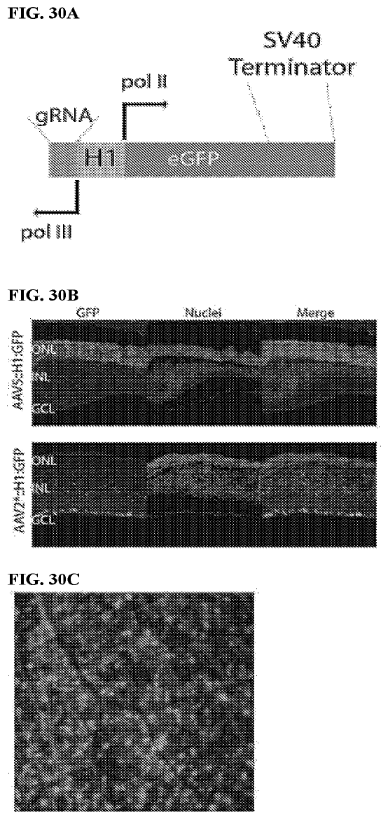

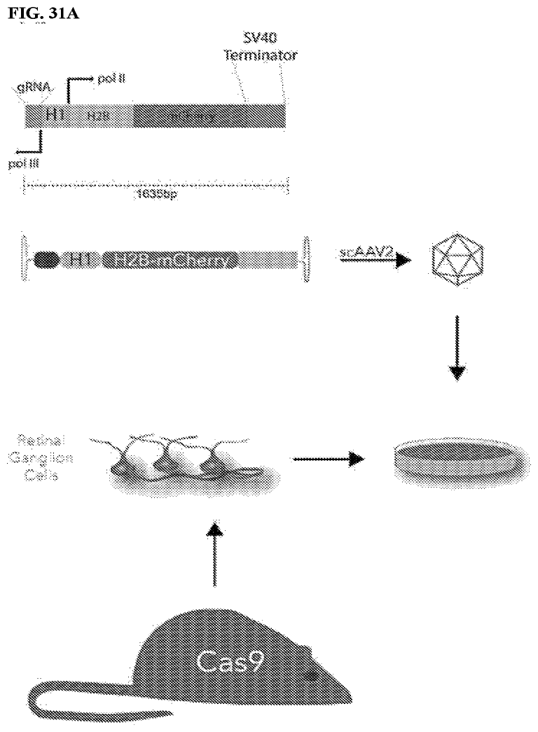

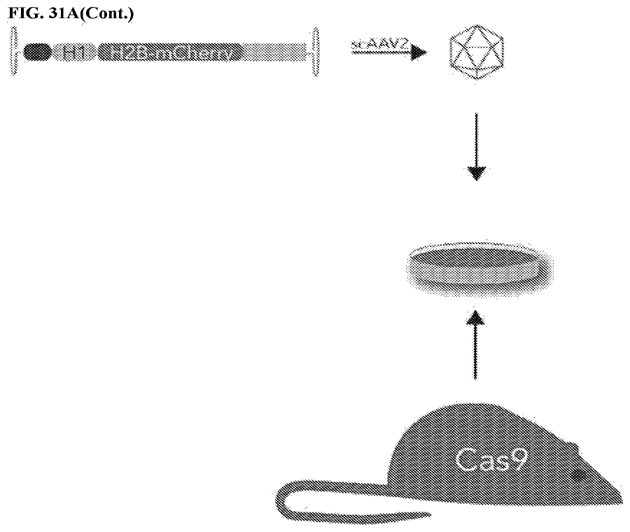

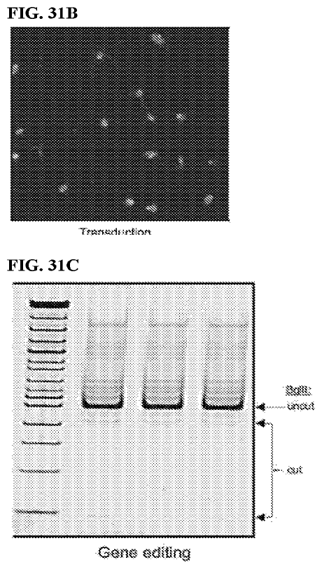

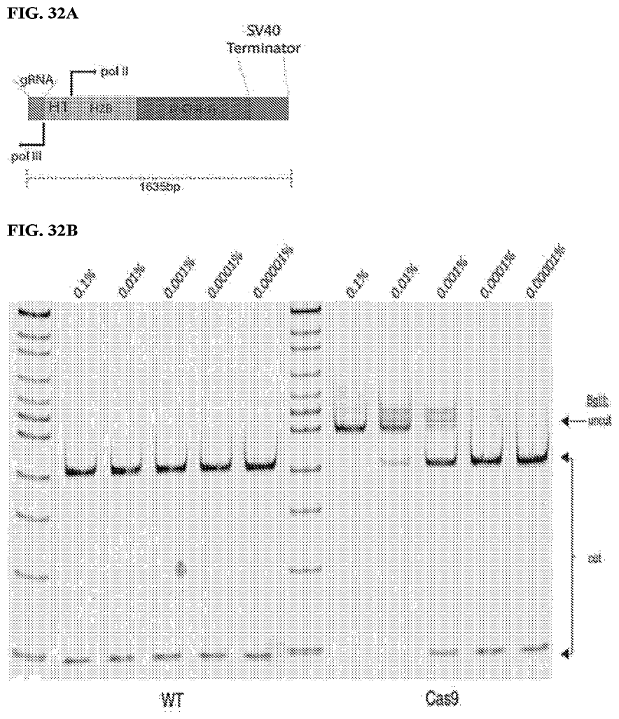

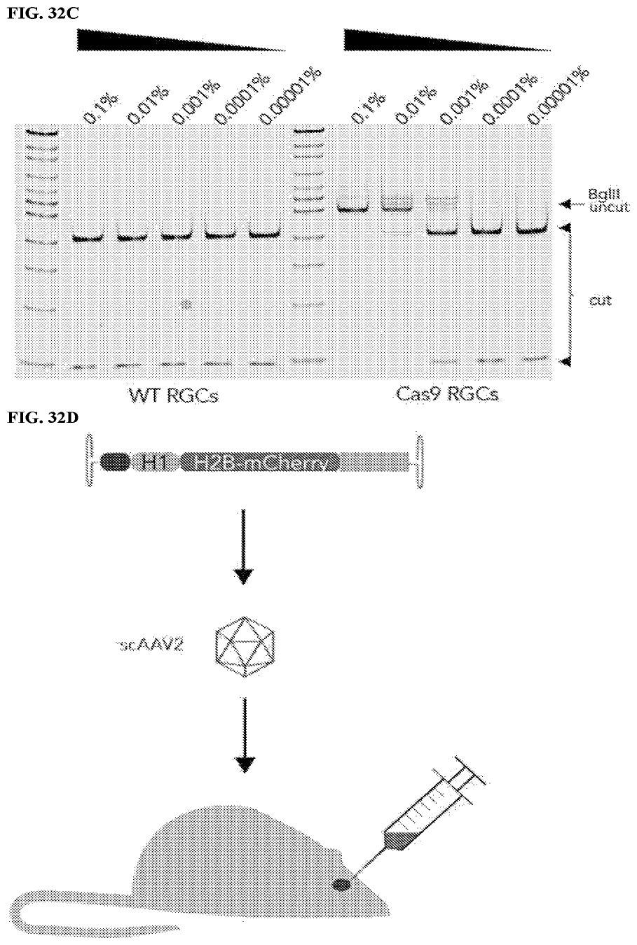

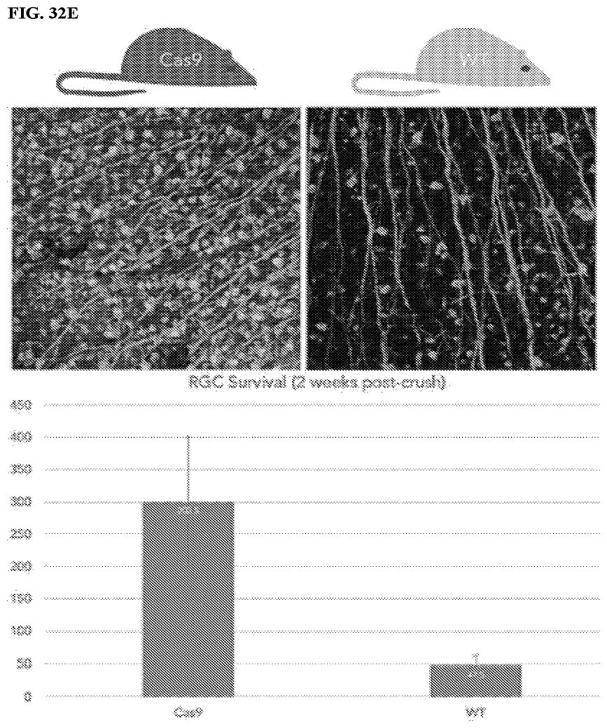

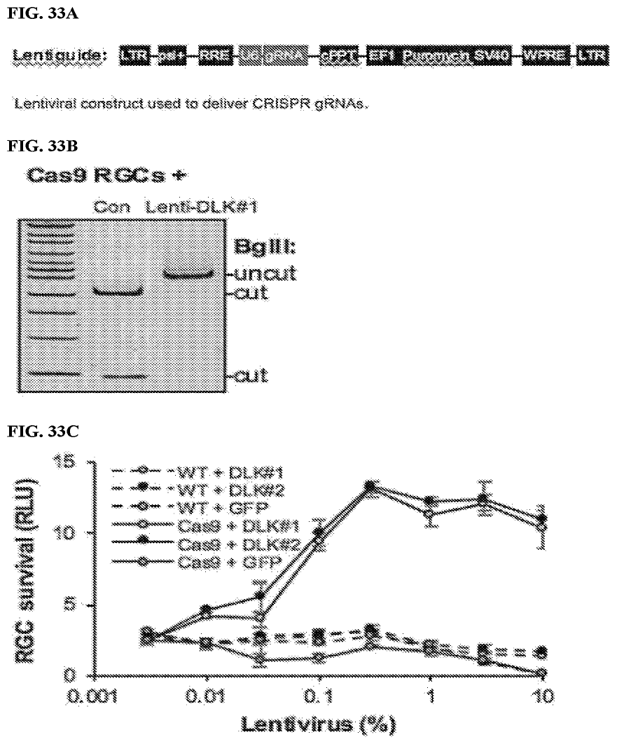

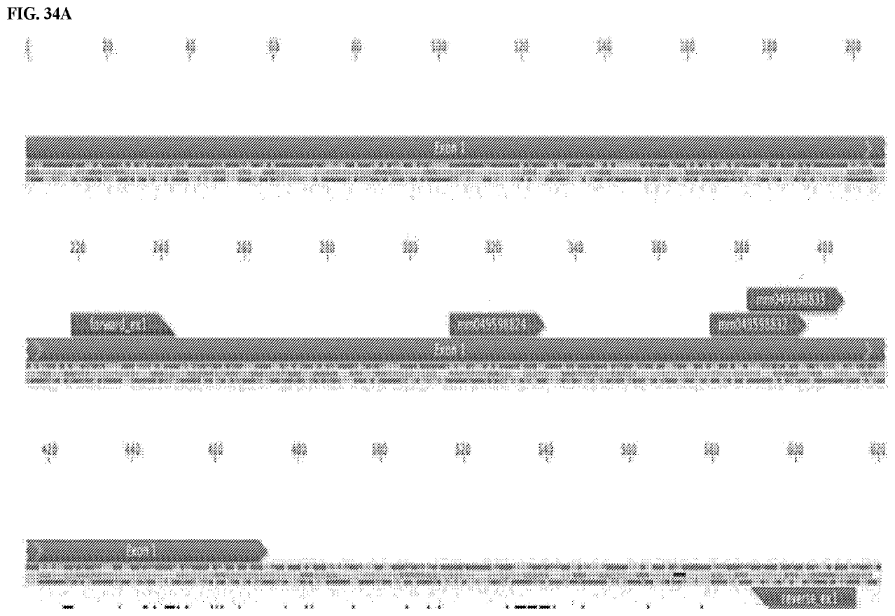

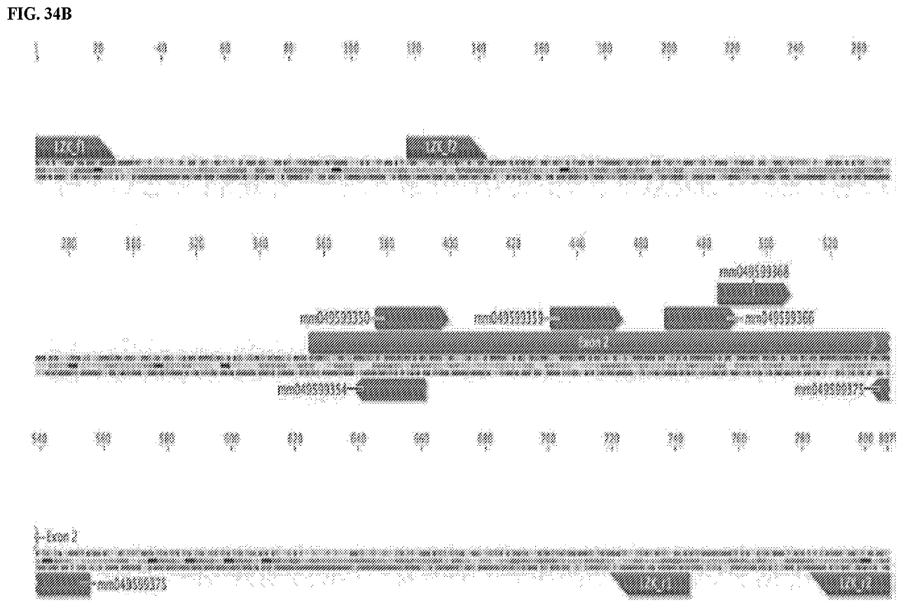

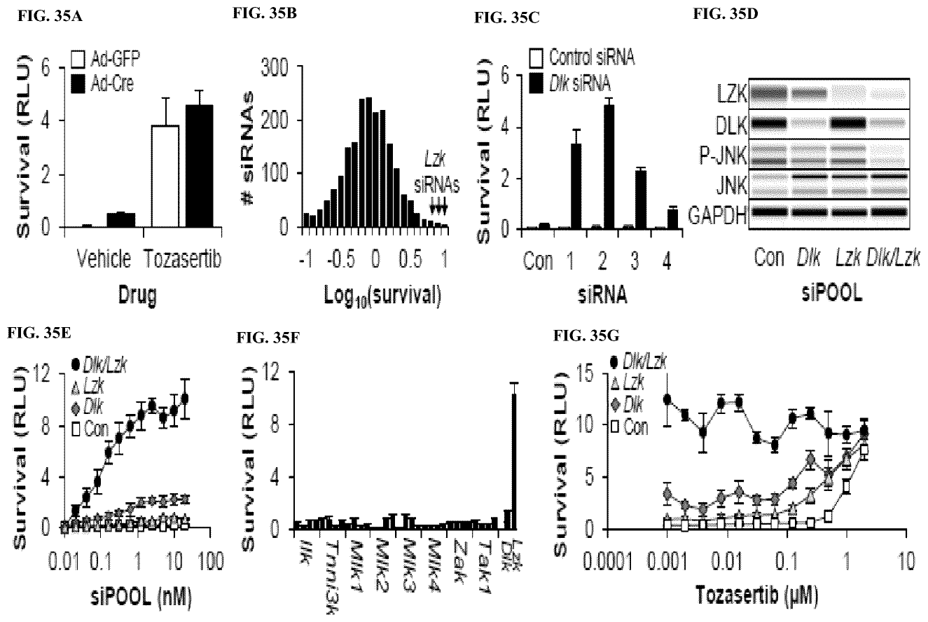

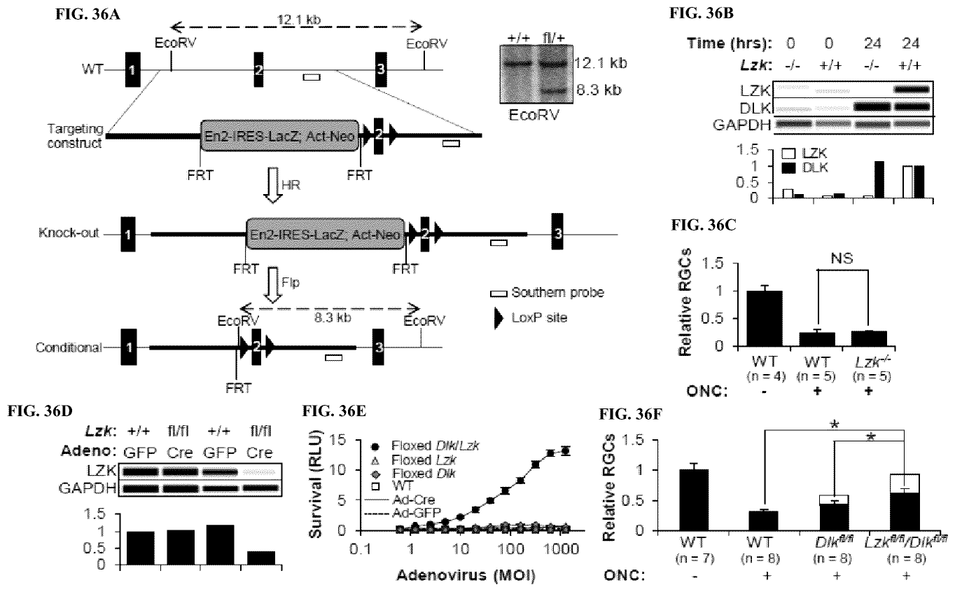

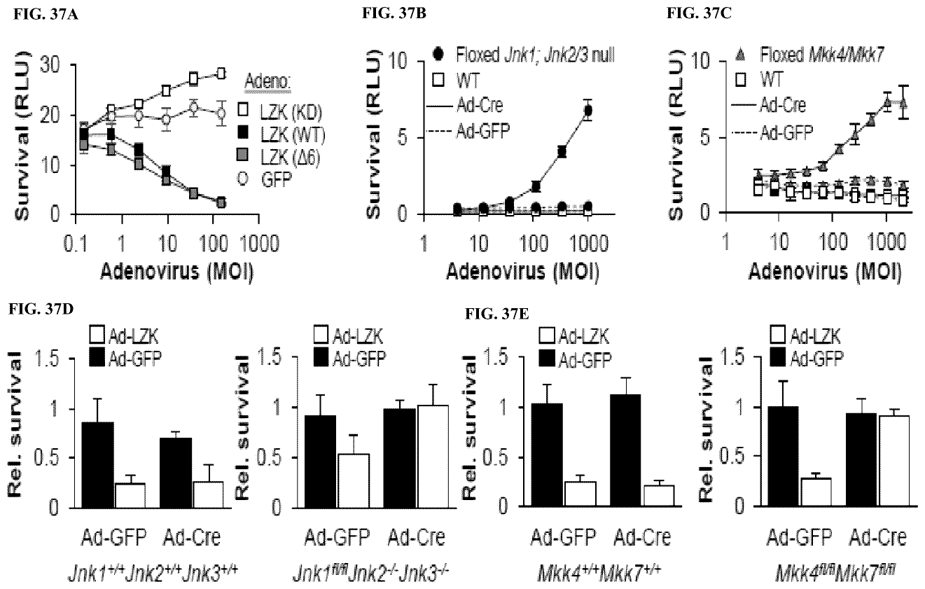

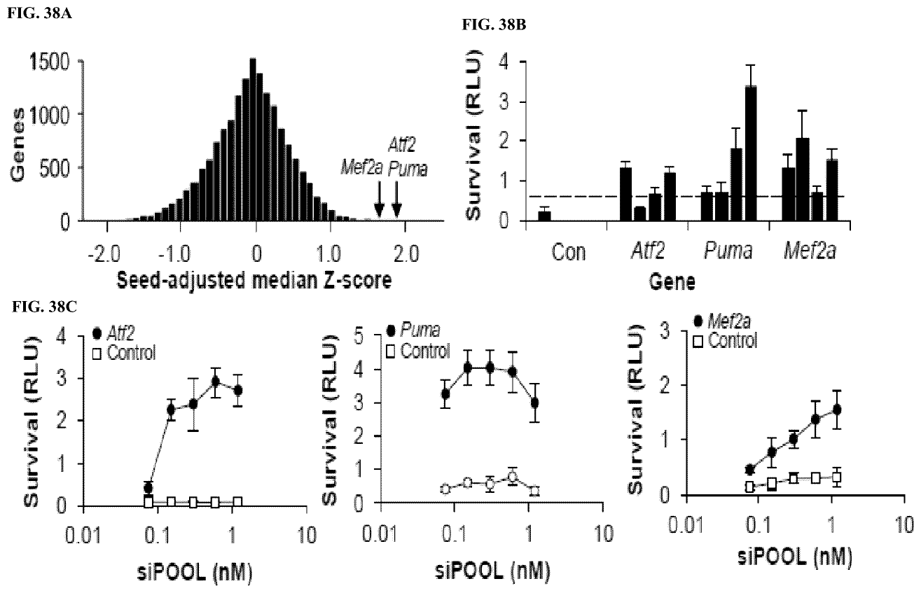

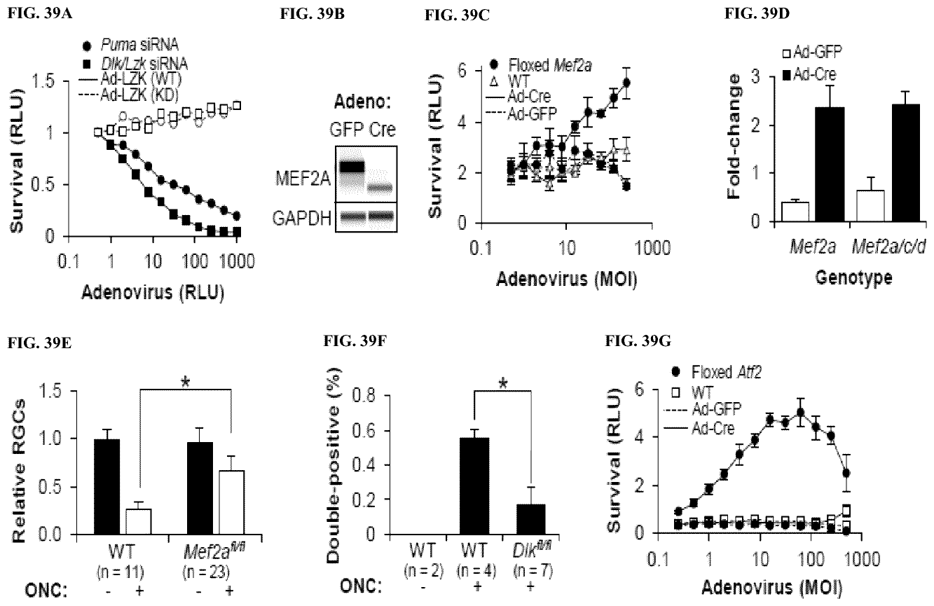

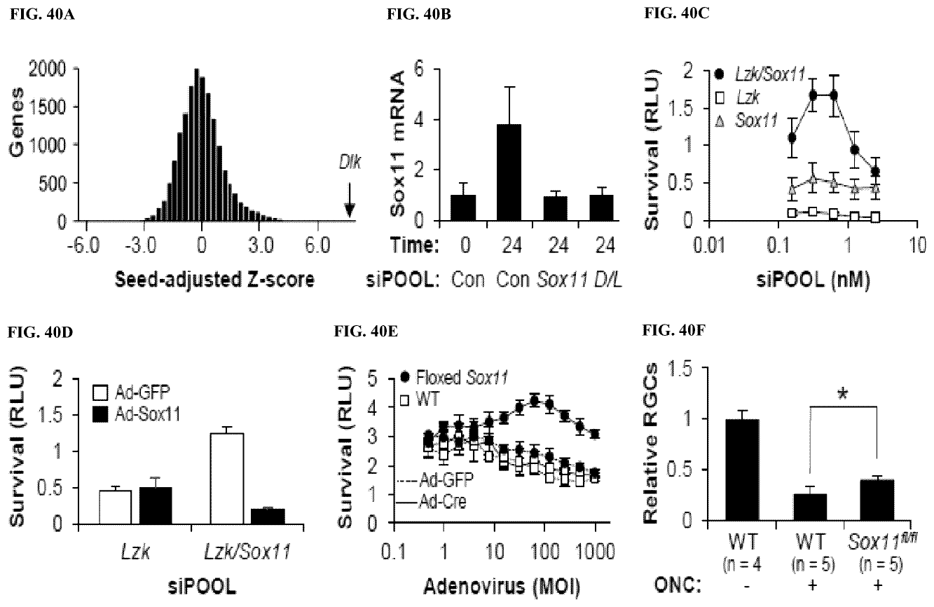

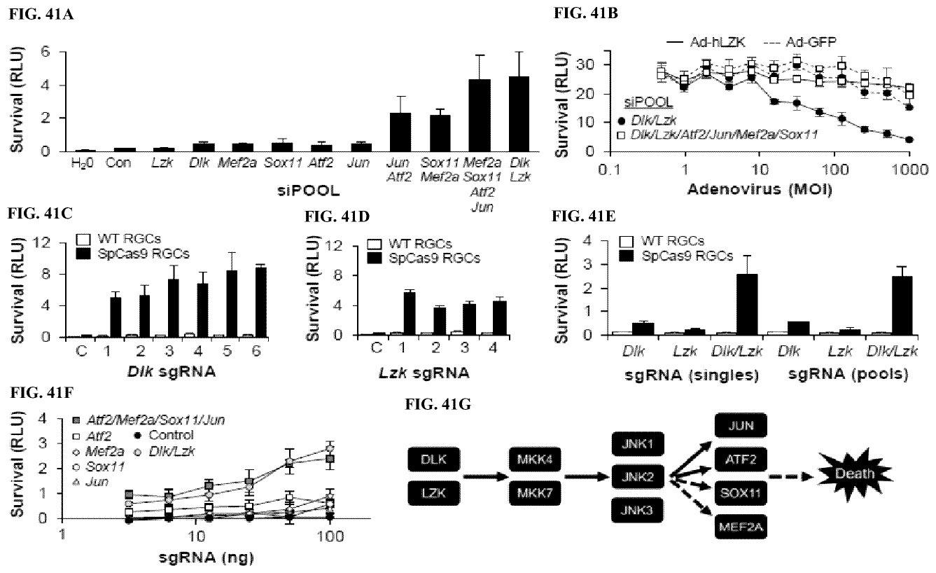

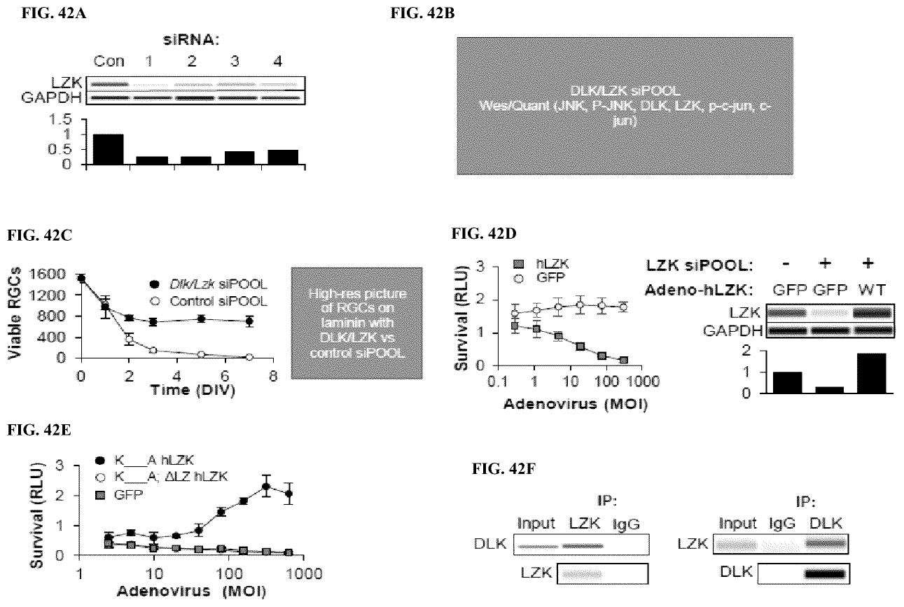

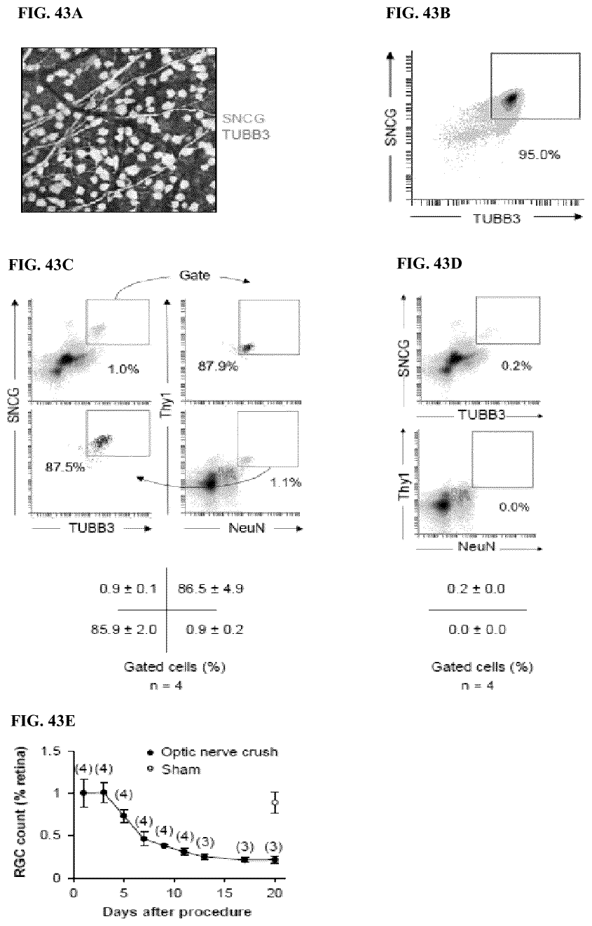

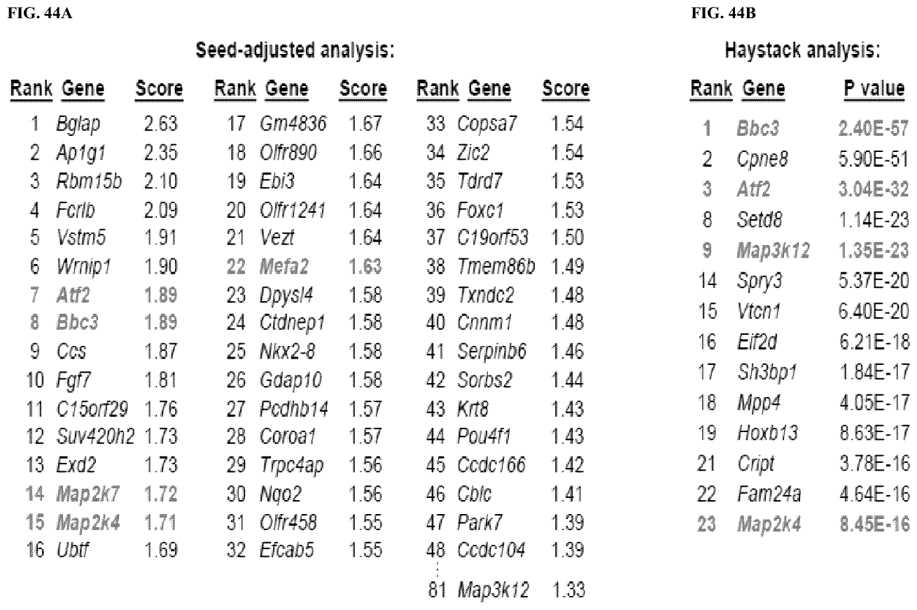

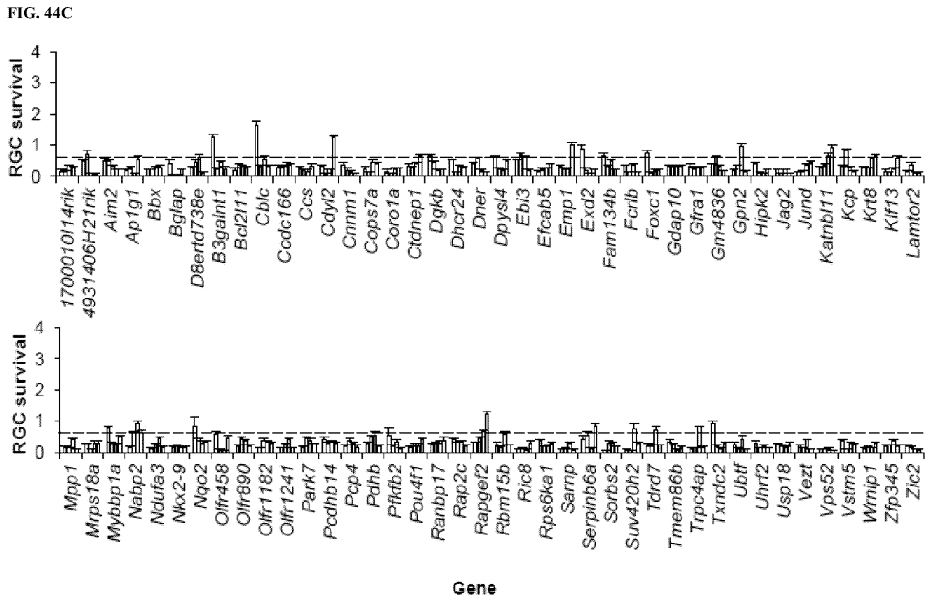

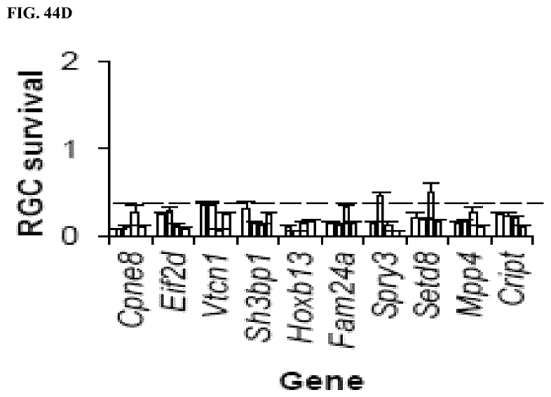

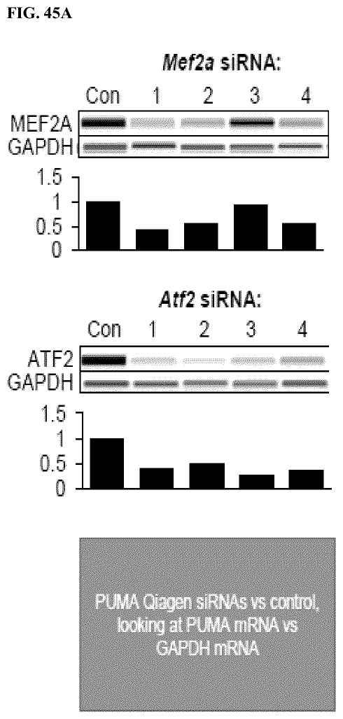

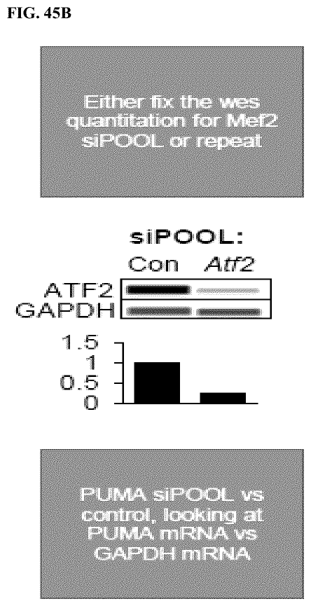

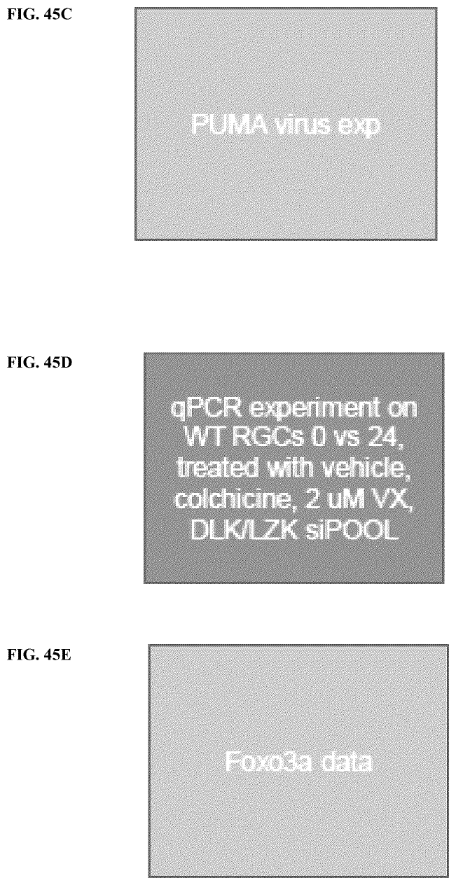

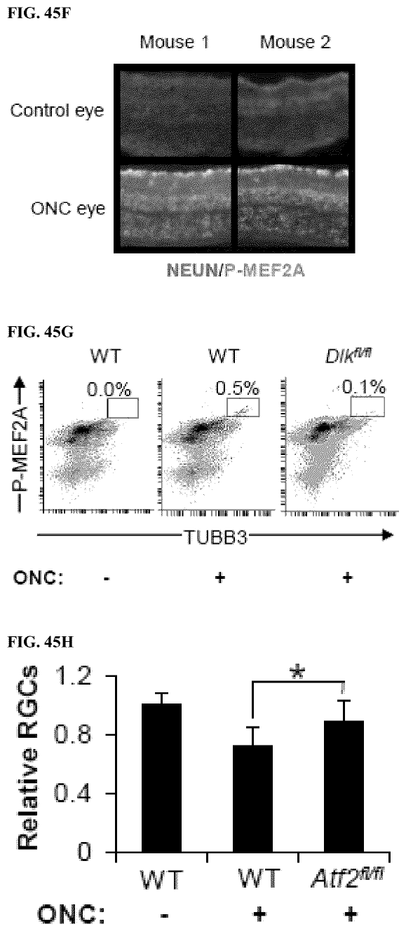

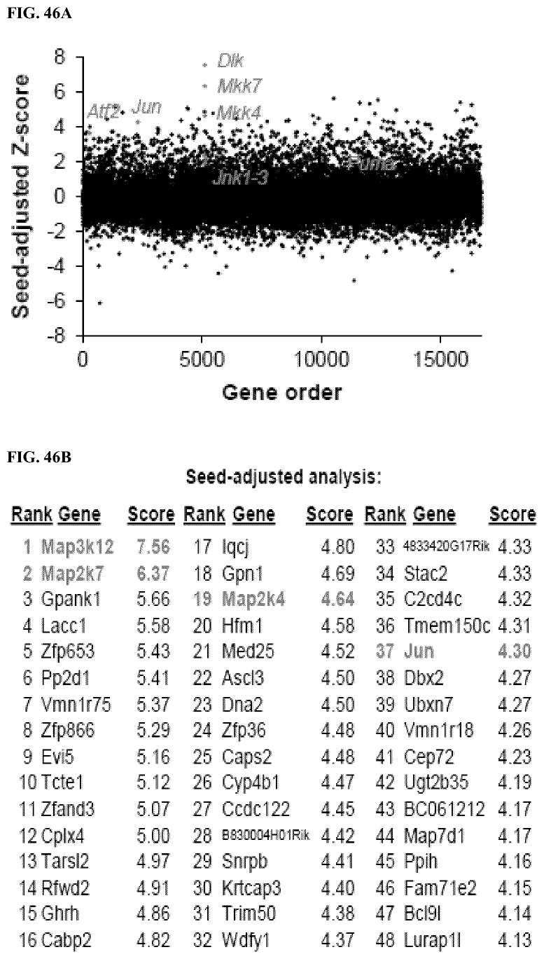

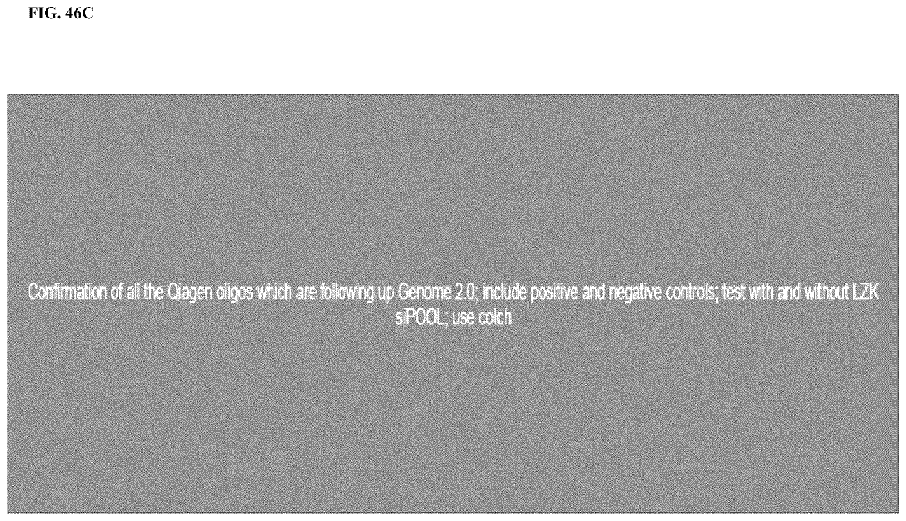

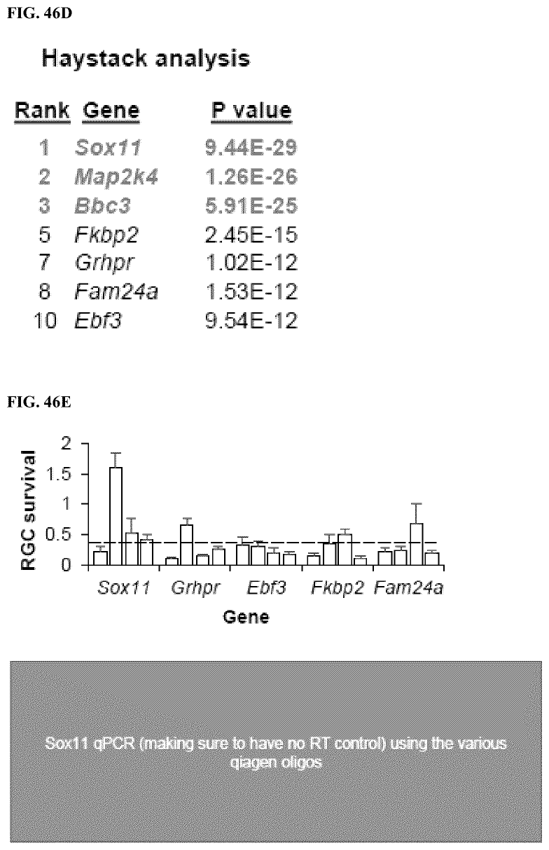

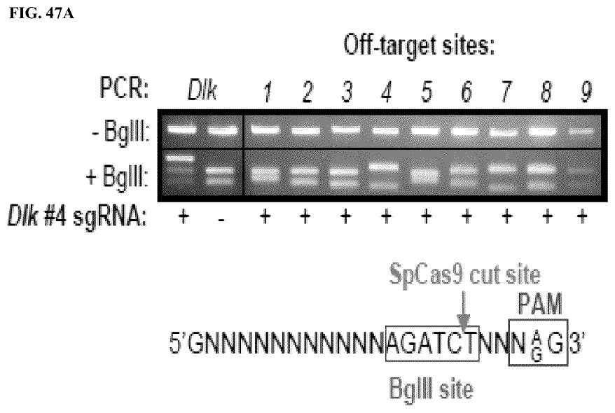

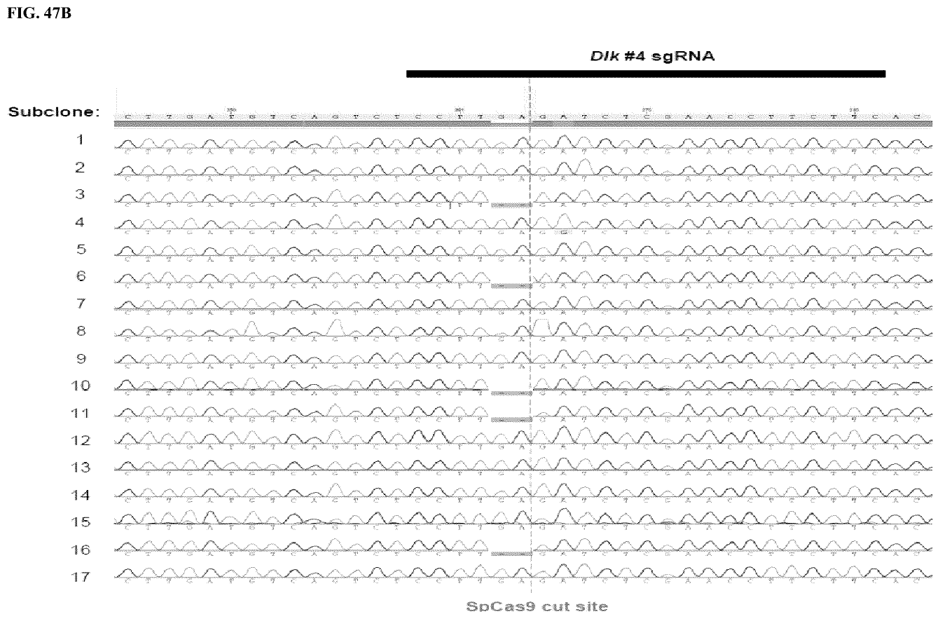

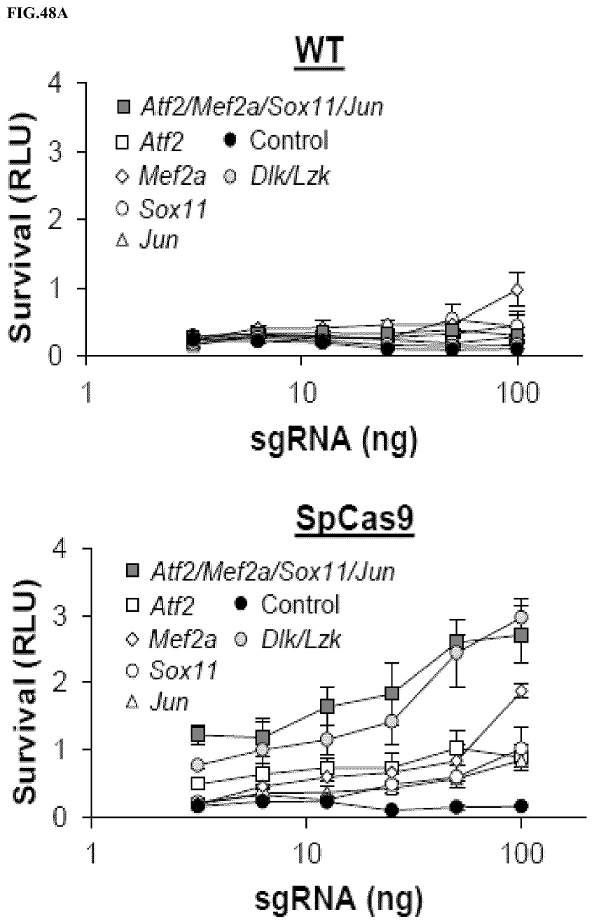

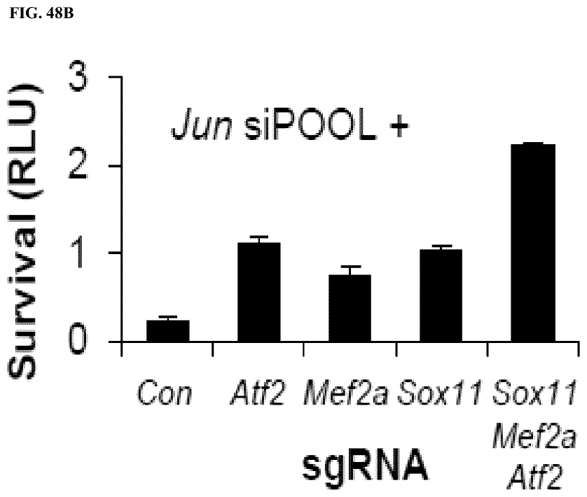

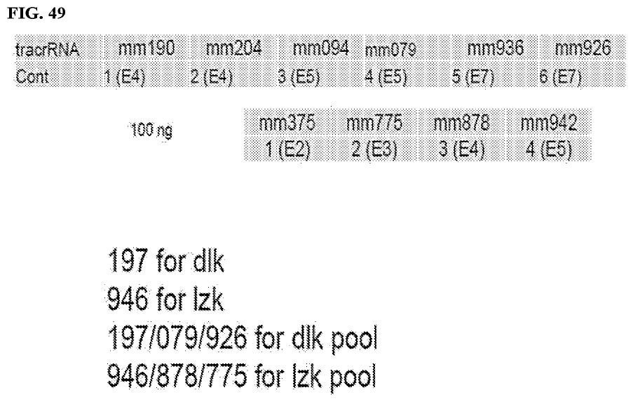

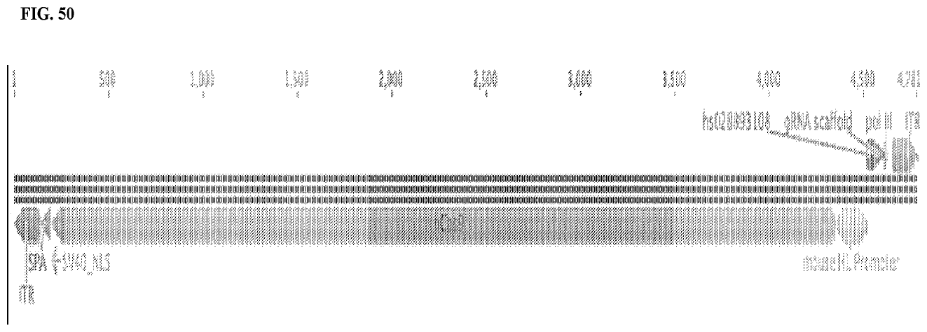

To help with the analysis of RGCs after transduction, a flow cytometry was developed based method for quantifying and characterizing mouse RGCs. Retinas are isolated, dissociated and fixed with acetone (to preserve nucleic acid quality) and then stained with antibodies against the RGC antigens γ-synuclein (SNCG) and β-TH-tubulin (TUJ1). The SNCG+/TUJ1+ double-positive cells represent the RGCs, as confirmed by the findings that they also stain for the additional RGC markers, Thy1.2 and NeuN (FIG. 26A), their survival is reduced after optic nerve crush (FIGS. 26B and 25C), and purified primary RGCs have a similar profile (FIG. 26B). Unlike Thy1.2 and NeuN, however, the expression of SNCG and TUJ1 is not downregulated by axonal injury (data not shown), and thus they remain useful markers in models of optic neuropathy. Finally, the method is compatible with cell sorting and it was shown that the sorted cells have a proteomic profile very similar to immunopanned RGCs, and intact RNA/DNA (data not shown).

7. Knockout Dlk as Proof-of-Principle that a scAAV/CRISPR-Based Approach can be Used to Target Genes In Vivo in the spCas9-Knockin Mouse.

Using the virus that we have validated in vitro (FIG. 25B), we will optimize the viral titer in vivo. Mice expressing spCas9-2A-GFP will receive unilateral intravitreal injections (˜1 μL) of the virus at 1010, 109, 108 or 107 viral particles/μL (four mice per group). After two weeks, which gives sufficient time for AAV life cycle completion, RGCs will be quantified and sorted as described above.

Finally, genomic DNA will be purified and assayed for Dlk disruption, as measured by the loss of BglII digestion, in order to determine the optimal titer (i.e. greatest amount of cutting without causing RGC toxicity). In the final step, mice expressing spCas9 will be intravitreally injected in one eye with the optimal dose of scAAV2-Dlk gRNA:H1:H2B-mCherry or a control virus that expresses a nontargeting gRNA in place of the Dlk gRNA. After two weeks, the eyes will undergo a three second optic nerve crush (so as not to compromise the vasculature) or

the sham control procedure and survival in the four groups (n=10 retinas per group) will be measured by SNCG/TUJ1 flow cytometry after an additional two weeks. In the future, whether this system is compatible will be tested with intravenous administration of AAV9 vectors, as this approach has been shown to effectively transduce RGCs (57, 58).

Based upon the already-described floxed Dlk mouse experiments, it is known that targeted disruption of Dlk improves RGC survival (5). Moreover, it is known that CRISPR-based knockout works in vitro. Thus, it seems highly likely that an increase in survival will be detected in those RGCs transduced with the Dlk gRNA. If >50% cutting or a survival phenotype is not seen, mCherry-expressing, AAV-transduced cells will be analyzed. This should be readily achievable because the flow cytometry technique currently works with as many as four channels (FIG. 26A).

8. Use the scAAV/CRISPR System to Validate that the Hits Identified in SA1 are Mediators of RGC Cell Death in the Mouse Optic Nerve Crush Model.

Identified genes whose knockdown or knockout improves RGC survival in vitro will be targeted in vitro with gRNAs and tested as described for DLK in Section 7. Priority will be given to those genes that appear to function upstream of DLK or that do not seem to affect regeneration. As was done with DLK, multiple gRNAs for each target gene will be tested in primary RGCs to identify the most efficacious ones to move forward into the AAV system. Finally, targets hypothesized to be upstream of DLK will be validated to determine if they suppress the pathway in vivo by immunostaining retinal flatmounts for phosphorylated JUN and DLK (5).

Genes arising from Section 1 should be straightforward to test in vivo as the screen identifies the active gRNAs. However, since the screen was sensitized with Lzk siPOOL, it is conceivable that the phenotype in vivo will require LZK or DLK inhibition. Fortunately, when the Lzk conditional knockout was made, a strategy was used that first produced a null allele. Moreover, unlike the situation with the Dlk-null mice (59), Lzk-null mice were viable and fertile. So, the Lzk-null mice were crossed with the spCas9-knockin line to produce Rosa26Cas9/Cas9Lzk−/− mice, the more appropriate line to confirm Section 1 hits.

Genes identified in Section 2 may be more challenging to validate in vivo as they will likely require simultaneous disruption of multiple genes. For those, the small size of the construct will be utilized, and the fact that an additional H1 driving expression of another gRNA can be accommodated, and still keep the same scAAV design. This will allow knockout of two genes with one viral vector (discussed more in Section 11). Future effort, could attempt to use more than two gRNAs (in place of mCherry or in a single-stranded (ss) AAV design) in order to target networks of genes.

9. Use a Modified scAAV2/CRISPR Approach to Validate that Hits Identified in Section 5 do not Affect Regeneration.

Basal levels of RGC regeneration after optic nerve crush are exceedingly low but can be enhanced by inhibition of PTEN, although not when PTEN and DLK are simultaneously inhibited (6). As one approach to test if inhibition of the candidates identified in Section 5 promotes RGC survival without affecting regeneration (unlike DLK), knockout mice were obtained for each candidate gene and breed them onto the Ptenfl/fl background—a prohibitive amount of time and effort. Instead, the in vivo knockout approach will be used to generate scAAV that express a gRNA targeting the gene-of-interest from the standard bidirectional H1 promoter in the Pol III direction and mCherry without the H2B fusion (in order to visualize axons) in the Pol II direction. The second H1 promoter will express a Pten gRNA. Mice expressing spCas9 will be intravitreally injected in one eye with the optimal dose of scAAV2-X gRNA:H1:mCherry; H1:Pten gRNA where X is either a nontargeting gRNA (negative control for preventing regeneration), the Dlk #4 gRNA (positive control for preventing regeneration) or a gRNA targeting a candidate from Section 5. After two weeks, the eyes will undergo a three second optic nerve crush (so as not to compromise the vasculature). Finally, after an additional two weeks, optic nerves from the three groups (n=10 optic nerves per group) will be harvested, longitudinally sectioned and the number of mCherry-positive axons extending beyond the crush site will be quantified.

Should the regeneration phenotype of Pten deletion be insufficient, the effect with Socs3 can be potentiated (33, 35, 60). These experiments are proposed as alternatives because the seminal work on the role of DLK in regeneration used Pten (and not combined Pten/Socs3) deletion (6). Multiallelic disruption is ambitious and may occur at a low frequency. Fortunately, the compact H1 promoters leave nearly 1 kb of packaging space, even with the two H1 promoters and a scAAV design, allowing one to engineer in fluorescent protein-based reporters for CRISPR editing (61).

10. Knockout Dlk as the Proof-of-Principle that a Therapeutically-Suitable, ssAAV/CRISPR Based Approach can be Used to Target Genes in a Wildtype Mouse.

The experiments outlined in Sections 7 and 8 take advantage of the robustness of scAAV and allow for quick validation of biology, but are not therapeutically useful as they rely on the spCas9 knockin mouse. As a complementary approach, the bidirectional feature of the H1 promoter will be used to drive the expression of Cas9 and gRNA, allowing both cassettes to be packaged into a single viral vector. Although the strategy allows for an insert that is comparable in size to wildtype AAV, it is too large for scAAV. Thus, ssAAV2-Dlk gRNA:H1:spCas9 particles will be prepared and tested as described in Section 7, except that the animals will not express spCas9 (since it is now being provided by the virus). The virus will be tested in a glaucoma model and include functional measures of vision. Importantly, while Dlk and RGCs have been the focus, these data would have an even more far-reaching implication—the use of a single AAV/CRISPR virus to modify the genome and increase the resistance to a disease state.

The percentage of cells undergoing biallelic disruption may be insufficient to reliably detect a survival increase in the optic nerve crush model. To date, nearly 20 Dlk target sites have been sampled using the in vitro model and have not found sites that work much better than #4 (used for Section 6). Even monoallelic disruption of Dlk can increase survival, albeit less than the robust protection conferred by biallelic disruption (37). To sensitize the cells to this monoallelic Dlk disruption, the experiment can be repeated using the Lzk-null mice. A complementary strategy would be to extend the time between optic nerve crush to RGC quantification. Although, the experiment is typically stopped at two weeks (when ˜75-80% of RGCs have died), ongoing loss occurs over the next 1-2 weeks, eventually reaching a plateau of ˜90-95% cell death. Even if only 5% of the cells have biallelic disruption of Dlk, that could produce a 50-100% relative increase in the number of surviving cells at one month (compared to the 20-25% increase at two weeks). Finally, although the Dlk #4 target site is preferred (because of the BglII site), the experiment could be repeated with #5 which has the exact same sequence in mice and humans and thus allows for the animal model to test a human therapeutic.

11. Measure the Off-Target Produced by the AAV/CRISPR Therapeutic Developed in Section 10. It is Well-Established that spCas9 Tolerates Mismatches Better in the Portion of the Protospacer that is Distal to the PAM (62, 63), Thus the Most Likely Off-Targets have a Conserved 3′ Sequence and 1-2 Mismatches in the 5′ End.

Since the BglII site is located near the cut site (3′), nearly all likely off-targets for the #4 gRNA retain the BglII site. The genomic DNA obtained in Section 10 can be used and the BglII assay described in FIG. 19H to quantify the cutting at the most likely off-target sites.

While the specificity in vitro (FIG. 19H) is encouraging, off-target cutting could be an issue. Should that be the case, one of two recently described sets of mutations (spCas9-HF1—N497A, R661A, Q695A, Q926A; eSpCas9—K810A K103A, R1060A) can be used in spCas9 which improve specificity (64, 65). An alternative would be to test another gRNA sequence, although measuring on-target and off-target cutting would have to be done with more laborious methods like the T7 endonuclease assays and sequencing. A variant of this idea would be to truncate 1-3 nucleotides off the 5′ end of the #4 gRNA, a technique that has been shown to improve specificity (66). Fortunately, the #4 gRNA starts with GAAG so any of these truncations are still compatible with the H1 promoter (which initiates at A or G). Finally, it is worth mentioning that H1-expressed gRNAs may produce lower rates of off-target cutting (53).

12. Develop an AAV/CRISPR Therapeutic that Delivers Cas9 and Two gRNAs in a Single Vector.

By using the H1 promoter, the Dlk gRNA:H1:spCas9 has a size of 4.5 kb, well under the 4.7-4.8 kb size of the wildtype virus and the 5.2 kb AAV packaging limit. This affords an opportunity to modify the construct proposed in Section 10 with an additional H1-gRNA cassette (˜0.2 kb). Expressing two gRNAs from a single virus (in addition to spCas9) opens up several possibilities, including the use of the more specific “nickase” mutant spCas9 (67), the generation of large deletions in a single gene and, relevant to the theme of our work, the simultaneous targeting of two compensatory genes. To test this, the H1:Lzk gRNA #1 will be added to expression cassette to the construct described in Section 10 and generate ssAAV2-Dlk gRNA4:H1:spCas9;H1:Lzk gRNA5 viral particles. These will be tested as described in Section 10, this time comparing viruses that target Dlk alone or in combination with Lzk (n=10 retinas per group).

The size of this construct pushes the limit of the packaging capacity of AAV (although remains less than 0.1 kb larger than wildtype virus). If this becomes an issue, the smaller mouse H1 promoter (which also functions bidirectionally) could be used or saCas9, which is nearly 1 kb smaller than spCas9 (52). The latter approach would require different target sites as neither the #4 nor #5 site is compatible with the saCas9 PAM requirement.

13. Explore the Mechanism by which DLK Regulates MEF2A to Promote RGC Cell Death.

MEF2A has a well-studied role in muscle differentiation and cardiovascular physiology (68). In neurons, MEF2A has been shown to play a critical role in promoting survival (27) and, although the brain-specific conditional knockout of Mef2a does not affect gross neuronal survival, the combined knockout of Mef2a/c/d shows a clear increase in neuronal apoptosis leading to early post-natal lethality (30). In response to excitotoxic stimuli, cortical neurons utilize two mechanisms to inhibit MEF2A signaling and promote apoptosis: a caspase catalyzed cleavage of MEF2A that results in dominant-negative activity (69) and a cyclin-dependent kinase 5 (CDK5) phosphorylation of S408 of MEF2A that leads to inactivation of the transactivation domain (70) or, especially in the case of dendritic/synaptic physiology, the subsequent sumoylation at K403 and the formation of a transcriptionally-repressive form of MEF2A (29, 71). In each of these cases, MEF2A promotes neuronal survival and inhibition of MEF2A leads to cell death. In contrast, MEF2A was identified as a mediator of RGC cell death in vitro and in vivo (FIGS. 20F and 20G). When the survival of floxed Mef2a RGCs was compared with floxed Mef2a/c/d RGCs (30), no difference was seen (data not shown) suggesting that MEF2A was not interfering with MEF2C/D to promote RGC cell death. Finally, although robust, DLK-dependent, MEF2A S408 phosphorylation were detected in response to optic nerve injury in vivo or immunopanning in vitro, a nonphosphorylatable S408A mutant does not interfere with activity (FIG. 20I). Together, these results suggest that DLK activation leads to novel MEF2A alterations that are responsible for cell death.

### 14. Use a Proteomic Approach to Identify the DLK-Dependent Post-Translational Modifications in MEF2A.

In order to identify the mechanism by which DLK/LZK regulates MEF2A, MEF2A was purified from floxed Dlk/Lzk primary RGCs transduced with adeno-Cre (in order to ablate DLK/LZK signaling), adeno-GFP (which have active DLK/LZK signaling), wildtype rat DLK (which superactivates the pathway) or kinase-dead rat DLK (which functions as a dominant-negative). Phosphorylated residues and the relative abundance of phosphorylation at each of these sites will be determined by tandem mass tag (TMT, Thermo Scientific) quantification and liquid chromatography tandem mass spectrometry (LC-MS/MS) analysis on one of several Orbitrap mass spectrometers (Thermo Scientific) available to our group. This approach also permits an analysis of other post-translational modifications like sumoylation and acetylation, already known to play a role in MEF2A signaling (29, 71). Changes that appear dependent on the presence of DLK/LZK and especially those that increase with superactivation of the pathway will be followed up in Section 15.

The biggest challenge may be the amount of starting material, given that MEF2A post-translational modifications will be assayed in the context of our disease relevant, primary cell system. To circumvent the problem, 3-5,000,000 RGCs can be routinely isolated, which should be more than enough given the low-complexity of the IP (i.e. only looking at one protein). If not enough MEF2A protein can be obtained, overexpression of an epitope-tagged MEF2A may be used. Second, the sensitivity and accuracy of MS analysis can be increased via multiple reaction monitoring (MRM) approaches targeting potential MEF2A phosphorylation sites—both known and unknown—after first optimizing an MRM assay for these species (72, 73). If phosphorylation at multiple simultaneous sites is critical for MEF2A regulation by DLK/LZK, a top-down proteomics approach can be employed on intact non-trypsinized MEF2A to determine multiplicity of phosphorylation.

### 15. Use a Combination of Mutations and Gain-of-Function/Loss-of-Function Experiments to Validate the Proteomic Ally-Identified Post-Translational Modifications.

Posttranslational modifications identified in Section 14 will be tested in the primary RGC system. Mutants for each of the residues (e.g. S408A) will be engineered into the mouse Mef2a cDNA and shuttled into an adenoviral vector as described multiple times above. RGCs will then be isolated from the floxed Mef2a mouse and transduced with adenovirus-Cre (MOI 1000) to promote survival and ablate endogenous MEF2A. After 48 hours, adenovirus will be used to reintroduce wildtype MEF2A or the unmodifiable mutants. If a post-translational modification is necessary for MEF2A-dependent killing, the mutant is expected to be a loss-of-function. Moreover, overexpression of wildtype DLK (which superactivates the pathway and accelerates RGC cell death) will be used in combination with the mutants to see if they possess dominant-negative activity (i.e. as would be expected if the modification were part of the mechanism by which DLK signaling activates MEF2A). Finally, at least in the case of phosphosites, phosphomimetic mutations (e.g. S408E) will be engineered and tested whether these gain constitutive activity and promote cell death even in the setting of combined DLK/LZK inhibition (using siRNA).

16. Use a CRISPR/AAV Strategy to Test Whether Targeted Disruption of Mef2a In Vivo Promotes RGC Survival and without Affecting Axonal Regeneration.

Although DLK plays a key role in both cell death and axon regeneration, four downstream transcription factors were identified that mediate the cell death signal. While at least two of them (i.e. SOX11 and JUN) have known roles in regeneration, the role of MEF2A is unknown. Thus, MEF2A could potentially represent a neuroprotective target that dissociates survival and regeneration. Indeed, in vitro, MEF2A knockdown does not appear to grossly affect neurite outgrowth (data not shown). Normally, this line of investigation would be of limited utility since MEF2A, being a transcription factor, is not readily druggable. However, the strategy outlined in Section 10 allows us to therapeutically target genes like MEF2A. Moreover, the approach described in Section 9, in which multiple genes were knocked out, and even

selectively label the cells that have active CRISPR editing, provides an opportunity to easily test MEF2A biology in vivo. Thus, spCas9-2A-GFP knockin mice will be transduced with the virus described in Section 9, modified to target Mef2a, Dlk (positive control for preventing regeneration) or a non-targeting control (negative control for preventing regeneration), and assayed as described in Section 9.

### Conclusion

In summary, described herein are sets of experiments aimed at further exploring the upstream and downstream mechanism by which DLK and LZK promote RGC cell death. Moreover, using CRISPR/AAV therapeutics, a platform has been developed that allows for rapid validation in vivo and, most importantly, a seamless transition to a highly-specific gene therapy-based therapeutic. Indeed, while there is tremendous excitement about CRISPR-based therapeutics for genome-editing, the field is limited by the fact that spCas9 and the gRNA cannot be simultaneously packaged into a single viral vector. Using the H1 promoter, this obstacle has been overcome. Using DLK as an example, genome modification can be used therapeutically in optic neuropathies.

Targets:
DLK
exon1:
mm048414154:
(SEQ ID NO: 143)
AGGTCCAGGATTTCCTCAAAGGG
mm048414155:
(SEQ ID NO: 144)
ATCTAGAGTTCGAGCTGATGAGG
mm048414202:
(SEQ ID NO: 145)
GTGGGCGTCAGGTCTTTCTCGGG
exon2
mm048414070:
(SEQ ID NO: 146)
ACTTGAAAGTGATGATGTTGGGG
mm048414079:
(SEQ ID NO: 147)
GAAGAAGGTTCGAGATCTCAAGG
mm048414081:
(SEQ ID NO: 148)
GGAGGAGGTAGCTGTGAAGAAGG
mm048414082:
(SEQ ID NO: 149)
GGGACGCTTCCACGGGGAGGAGG
mm048414086:
(SEQ ID NO: 150)
GTTTTCCTGGGACGCTTCCACGG
mm048414094:
(SEQ ID NO: 151)
AGGTCCAGGATTTCCTCAAAGGG
LZK
exon1:
mm049598783:
(SEQ ID NO: 152)
ACGAAATGAGCTTGCAGCTATGG
mm049598824:
(SEQ ID NO: 153)
GTGGATGGAGAGAACACGAACGG
mm049598832:
(SEQ ID NO: 154)
GGCAGCGGCGGGTTTCTCGAAGG
mm049598833:
(SEQ ID NO: 155)
GGGTTTCTCGAAGGACTGTTTGG
exon2
mm049599350:
(SEQ ID NO: 156)
AGGAAATCTCAGAGCTGCAATGG
mm049599375:
(SEQ ID NO: 157)
GCAAGTGCCGACCTACTTGAAGG
mm049599764:
(SEQ ID NO: 158)
GACCTCGTACAGCTGTCCGTGGG
mm049599772:
(SEQ ID NO: 159)
GCAGCCGGGGCGTGATCTTCCGG
mm049599878:
(SEQ ID NO: 160)
GGATGGCACCAGAGGTGATCCGG
mm049599881:
(SEQ ID NO: 161)
GATCCGGAATGAGCCTGTCTCGG
mm049599884:
(SEQ ID NO: 162)
GGCTCATTCCGGATCACCTCTGG

### Example 5

### Protocols

HEK293 cells were seeded in T25 flasks and grown to semi-confluence in DMEM supplemented with 10% fetal bovine serum and antibiotics. Two days after plating, cells in one flask were transfected with the plasmid pAAV-CEP290. A transfection mixture containing 4 ug plasmid DNA, 20 ul Lipofectamine 3000 (ThermoFisher/Invitrogen) and 10 ul of the P3000 reagent (ThermoFisher/Invitrogen) in a total of 500 ul additive-free OptiMEM medium was added to the cell culture medium. Cells in a second flask were infected with 100 ul of a packaged and purified stock of AAV2-CEP290 (2.06e10 viral genomes). A third T25 flask was untreated and served as a control. Cells were harvested in trypsin-EDTA 48 h following transfection or infection, and pelleted by centrifugation. For each sample, the cell pellet was resuspended in 200 ul PBS and processed with the Qiagen DNA mini kit as per manufacturer's protocol. Genomic DNA was eluted in 200 ul water.

For T7 EndoI analysis, genomic DNA was extracted by resuspending cells in QuickExtract solution (Epicentre, Madison, WI), incubating at 65° C. for 20 min, and then at 98° C. for 20 min. The extract solution was used directly or cleaned using DNA Clean and Concentrator (Zymo Research, Irvine, CA), and quantitated by NanoDrop (Thermo 30 Fisher Scientific). The genomic region surrounding the CRISPR target site was amplified from ˜100 ng of genomic DNA using Phusion DNA polymerase (New England Biolabs). Multiple independent PCR reactions were pooled and purified using DNA Clean and Concentrator (Zymo Research, Irvine, CA). A 25 μl volume containing 150 ng of the PCR product in 10 mM Tris-HCl, 50 mM NaCl, 10 mM MgCl₂ and 100 μg/ml BSA was denatured and slowly reannealed to allow for the formation of heteroduplexes: 95° C. for 10 min, 95° C. to 85° C. ramped at −1.0° C./sec, 85° C. for 1 sec, 85° C. to 75° C. ramped at −1.0° C./sec, 75° C. for 1 sec, 75° C. to 65° C. ramped 5 at −1.0° C./sec, 65° C. for 1 sec, 65° C. to 55° C. ramped at −1.0° C./sec, 55° C. for 1 sec, 55° C. to 45° C. ramped at −1.0° C./sec, 45° C. for 1 sec, 45° C. to 35° C. ramped at −1.0° C./sec, 35° C. for 1 sec, 35° C. to 25° C. ramped at −1.0° C./sec, and then held at 4° C. 1 μl of T7 EndoI (New England Biolabs) were added to each reaction, incubated at 37° C. for 30 min, and then immediately placed on ice. For gel analysis, 3 μl of the reaction was mixed with 3 μl 2× Loading dye (New England Biolabs), loaded onto a 6% TBE-PAGE gel, and stained with SYBR Gold (1:10,000) for ˜15 minutes prior to visualization. Gels were visualized on a Gel Logic 200 Imaging System (Kodak, 15 Rochester, NY), and quantitated using ImageJ v. 1.46. NHEJ frequencies were calculated using the binomial-derived equation:

$$\% gene modification = 100 * ( 1 - ( SQRT ( 1 - ( ( a + b ) / ( a + b + c ) ) ) ) )$$

where the values of “a” and “b” are equal to the integrated area of the cleaved fragments after background subtraction and “c” is equal to the integrated area of the un-cleaved PCR product after background subtraction.

For restriction analysis, genomic DNA was extracted by resuspending cells in QuickExtract solution (Epicentre, Madison, WI), incubating at 65° C. for 20 min, and then at 98° C. for 20 min. The extract solution was used directly or cleaned using DNA Clean and Concentrator (Zymo Research, Irvine, CA), and quantitated by NanoDrop (Thermo 30 Fisher Scientific). The genomic region surrounding the CRISPR target site was amplified from ˜100 ng of genomic DNA using Phusion DNA polymerase (New England Biolabs). Multiple independent PCR reactions were pooled and purified using DNA Clean and Concentrator (Zymo Research, Irvine, CA). A 25 μl volume containing 150 ng of the PCR product in 10 mM Tris-HCl, 50 mM NaCl, 10 mM MgCl₂ and 100 μg/ml BSA was digested at 37° C. for 1 hr. For gel analysis, 3 μl of the reaction was mixed with 3 μl 2× Loading dye (New England Biolabs), loaded onto a 6% TBE-PAGE gel, and stained with SYBR Gold (1:10,000) for ˜15 minutes prior to visualization. Gels were visualized on a Gel Logic 200 Imaging System (Kodak, 15 Rochester, NY), and quantitated using ImageJ v. 1.46. NHEJ frequencies were calculated using the equation:

$$\% gene modification = ( ( a + b ) / ( a + b + c ) ) * 100$$

where the values of “a” and “b” are equal to the integrated area of the cleaved fragments after background subtraction and “c” is equal to the integrated area of the un-cleaved PCR product after background subtraction.

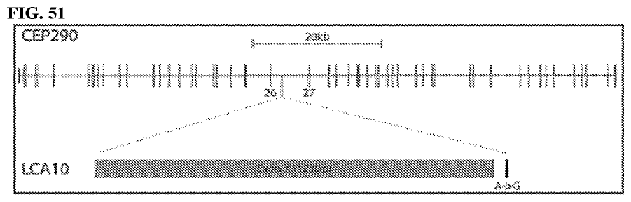

Leber's congenital amaurosis (LCA) is comprised of a group of early-onset childhood retinal dystrophies that are characterized by severe retinal dysfunction and severe visual impairment, or blindness during the first months of life. LCA, an orphan disease, is the most common cause of inherited blindness constituting as much as 5% of all known hereditary retinal degenerative diseases. The most common cause for LCA10—a disease for which no FDA approved therapy exists—is a deep intronic mutation in the CEP290 gene (den Hollander, A. I. et al. (2006) American journal of human genetics 79, 556-561) (FIG. 51). The IVS26 c.2991+1655 A>G mutation results in a de novo strong splicedonor site and the subsequent inclusion of a cryptic exon (Exon X) into the CEP290 mRNA. CEP290, centrosomal protein located in the connecting cilium of photoreceptors, plays a role in ciliogenesis and ciliary trafficking (Drivas, T. G. et al. (2013) The Journal of clinical investigation 123, 45254539; Craige, B. et al. (2010) The Journal of cell biology 190, 927-940; Tsang, W. Y. et al. (2008) Developmental cell 15, 187-197). Inclusion of Exon X, which contains a premature stop codon, results in a truncated form of CEP290 and eventual photoreceptor degeneration.

Some forms of LCA are potentially amenable to treatment by recombinant adeno-associated viruses (AAV) engineered to deliver a functional copy of the defective cellular gene. In 2008, a transgene that complemented the mutation in RPE65 was successfully delivered by AAV to LCA2 patients in a Phase I Clinical trial (Maguire, A. M. et al. (2008) The New England journal of medicine 358, 2240-2248). Some responses were noted, but unfortunately, the effects were not durable, potentially due to losing transgene expression (Azvolinsky, A. (2015) Nat Biotechnol 33, 678; Schimmer, J. et al. (2015) Human gene therapy. Clinical development 26, 208-210). Furthermore, mutations in some genes that cause the different LCA subtypes, like CEP290, are simply too large for AAV delivery, and therefore remain untreatable by gene therapy approaches. Moreover, photoreceptors are highly sensitive to protein levels (Olsson, J. E. et al. (1992) Neuron 9, 815-830; Tan, E. et al. (2001) Investigative ophthalmology & visual science 42, 589-600; Seo, S. et al. (2013) Investigative ophthalmology & visual science 54), and transgenic experiments have demonstrated photoreceptor toxicity from CEP290 overexpression (Burnight, E. R. et al. (2014) Gene therapy 21, 662-672). Given the following properties, we believe that the CEP290 mutation is a particularly attractive as a therapeutic target for CRISPR-Cas9 technology.

The development of CRISPR-Cas9 technology has revolutionized the field of gene-editing and offers a profoundly new approach to treating genetic diseases. The CRISPR-Cas9 system is composed of a guide RNA (gRNA) that targets the Cas9 nuclease in a sequence-specific fashion. Cleavage by the CRISPR system requires complementary base pairing of the gRNA to a DNA sequence and the requisite

protospacer-adjacent motif (PAM), a short nucleotide motif found 3′ to the target site (Doudna, J. A. et al. (2014) Science 346, 1258096; Hsu, P. D. et al. (2014) Cell 157, 1262-1278). Currently, the least restrictive and most commonly used Cas9 protein is from S. pyogenes, which recognizes the sequence NGG, and thus, the CRISPR targeting sequence is N20NGG. While numerous studies have shown that disease mutations can be efficiently targeted in vitro, the development of CRISPR-Cas9-based therapeutics for in vivo use is been hampered by safety concerns and delivery constraints.

While CRISPR targeting of disease mutations has been shown to be effective in numerous in vitro settings, and as well in vivo through mouse and other animal studies, all current approaches are still far from clinical use due in large part to delivery constraints. AAV vectors are the most frequently and successfully used viral vectors in ocular gene therapy injection (Dalkara, D. et al. (2014) Comptes rendus biologies 337, 185-192; Day, T. P. et al. (2014) Advances in experimental medicine and biology 801, 687-693; Willett, K. et al. (2013) Frontiers in immunology 4, 261; Dinculescu, A. et al. (2005) Human gene therapy 16, 649-663). Several features make AAV the most attractive choice: the virus is nonpathogenic, it infects both dividing and non-dividing cells, expression can persist for long periods of time, and it is particularly noteworthy for its history of safety, efficacy and a general lack of toxicity in clinical trials. Additionally, specific AAV serotypes are effective in targeting photoreceptor cells after subretinal injection. While AAV vectors provide a safe means of delivering therapeutic CRISPR components, there is one major technical obstacle that limits their utility—their size. Wild type AAV genomes are ˜4.7 kb in length and recombinant viruses can package up to ˜5.0 kb (Dong, B. et al. (2010) Molecular therapy: the journal of the American Society of Gene Therapy 18, 87-92; Wu, Z. et al. (2010) Molecular therapy: the journal of the American Society of Gene Therapy 18, 80-86). This packaging capacity defines the upper limit of the DNA that can be used for a single viral vector.

The DNA required to express Cas9 and the gRNA, by conventional methods, exceeds 5.2 kb: Pol II promoter (˜0.5 kb), SpCas9 (˜4.1 kb), Pol II terminator (˜0.2 kb), U6 promoter (˜0.3 kb), and the gRNA (˜0.1 kb). One approach to AAV delivery challenge is a two-vector approach: one AAV vector to deliver the Cas9, and another AAV vector for the gRNA (Swiech, L. et al. (2015) Nat Biotechnol 33, 102-106). However, the double AAV approach utilizes the small mouse Mecp2 promoter, a gene that has been found to be expressed in retinal cells—with the critical exception of rods (Song, C. et al. (2014) Epigenetics & chromatin 7, 17; Jain, D. et al. (2010) Pediatric neurology 43, 35-40)—suggesting that, aside from the potential toxicity due to increased viral delivery load, the co-delivery approach would likely fail to target the vast majority of LCA mutations apriori. While this is a potentially viable approach for other gene therapy-mediated genomic editing, we are instead proposing a single vector approach for retinal gene editing that should increase efficiency, target photoreceptors specifically, and reduce potential toxicity from viral load delivery.

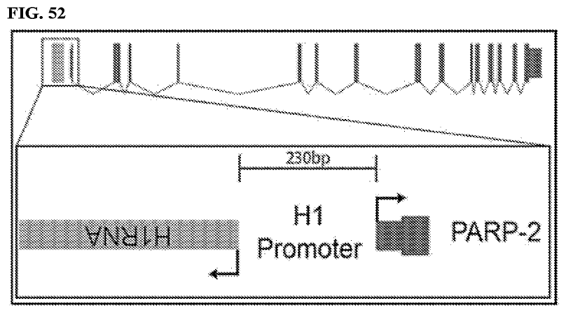

We recently reported that use of the H1 promoter, rather than the more traditionally used U6 promoter, to direct gRNA transcription allows an approximate doubling of the available CRISPR gene targeting space (Ranganathan, V. et al. (2014) Nature communications 5, 4516). Notably, we also detected a lower propensity for off-target cutting suggesting that the H1 promoter would be more favorable for therapeutic approaches. During these studies, we noticed the presence of a protein-coding gene (PARP-2) in close genomic proximity to the endogenous H1RNA gene (Myslinski, E. et al. (2001) Nucleic Acids Res 29, 2502-2509; Baer, M. et al. (1990) Nucleic Acids Res 18, 97-103). The sequence between the start of the H1RNA (a pol III RNA transcript) and the PARP-2 gene (a pol II transcript) is 230 bp (FIG. 52), indicating that this relatively small sequence can function as a compact bidirectional promoter—this is, to our knowledge, the only bidirectional promoter sequence in mammalian genomes that can direct both a pol 11 and a pol III transcript. We hypothesized that these fortuitous properties of the H1 promoter might allow us to overcome the size hurdles of packaging both CRISPR components into a single AAV.

We will develop two CRISPR/AAV therapeutics in parallel based on orthogonal Cas9 systems. First is the development of an LCA10 therapeutic mediated by the co-delivery of the SpCas9 and a guide RNA through a single AAV vector. Second is the development of an LCA10 therapeutic mediated by the co-delivery of the SaCas9 nickase and four guide RNAs through a single AAV vector. Both strategies are being developed for eventual clinical use and thus safety is paramount. Lastly, we will generate isogenic human stem cells lines containing the LCA10 for the characterization and development of novel therapeutics.

Development of an LCA10 Therapeutic Mediated by the Co-Delivery of the S. pyogenes Cas9 (SpCas9) and a Guide RNA Through a Single AAV Vector.

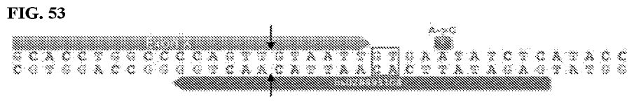

Background and Rationale. Although numerous studies have shown that disease mutations can be efficiently targeted in vitro, the development of SpCas9-based therapeutics for in vivo use has been hampered by delivery constraints. Using the compact bidirectional promoter system, we have demonstrated a clinically viable platform for the co-delivery of SpCas9 and a gRNA through a single AAV vector. SpCas9 offers several advantages: it is the most commonly used, most versatile, and best understood CRISPR system. Its PAM requirement (NGG) is considerably less stringent than other Cas9 proteins, which in turn means that more genes and more mutations can be directly targeted. Importantly for clinical therapeutic approaches, recent advances in protein engineering of SpCas9 have developed multiple high-specificity/high-fidelity variants with as little as zero detectable genome-wide off-target effects (Kleinstiver, B. P. et al. (2016) Nature doi:10.1038/nature16526; Slaymaker, I. M. et al. (2016) Science 351, 84-88). Indeed, unlike other Cas9 ortholog targeting sites, the intronic CEP290 mutation falls within an SpCas9 site (FIG. 53), which makes the LCA10 mutation a particularly attractive target for SpCas9.

By customizing the gRNA sequence, we can direct SpCas9 (or SpCas9 variant) to the CEP290 mutation, causing a double strand-DNA break near the splice-donor site. Cellular response to DNA breaks occurs primarily through one of two competing pathways: nonhomologous end-joining (NHEJ), or Homology Directed Repair (HDR). NHEJ, the more dominant pathway for DNA repair, is an error-prone pathway that results in deletions and insertions near the break point, commonly +/−˜15 nt (Mali, P. et al. (2013) Science 339, 823-826). Thus, by using the cells normal DNA repair machinery, we can disrupt the sequences near the splice-donor site, prevent inclusion of the exon X, and restore normal CEP290 splicing and function. Importantly, many mutations in this intronic region would be expected to restore in normal splicing.

Our approach will involve modifying and optimizing CRISPR-Cas9 methodology so all the needed components can be delivered to photoreceptors by a single AAV5, an AAV serotype with documented performance in mammalian rods. By swapping Cas9 for GFP in using our H1-AAV system, we have been able to demonstrate efficient delivery of GFP to photoreceptors using AAV5 (FIG. 2). Because LCA10 is recessive, a 50% rescue of normal CEP290 splicing should be sufficient photoreceptor function.

### Experimental Design and Methods.

1. Computational selection and analysis of gRNAs. Since the constructs are being developed with the goal of eventual clinical use, it is essential to carefully monitor them for potential off-target activity (Wu, X. et al. (2014) Quantitative biology 2, 59-70). For this purpose, we will pursue several complementary approaches. Taking a bioinformatics approach, we determined all the potential CRISPR sites in the human genome using a custom Perl script written to search both strands and overlapping occurrences of the SpCas9 targeting site; for example, in the human genome there are 137,409,562 CRISPR sites after filtering out repetitive sequences. We have computationally determined the propensity for each site to exhibit off-target effects using Bowtie (Langmead, B. et al. (2009) Genome biology 10, R25) to realign each CRISPR site back onto the genome allowing up to 3 base mismatches throughout the targeting sequence. Our analysis of the LCA10 SpCas9 site identifies 13 potential off-target loci that we will test for spurious targeting: 1 site with 2 mismatches, and 12 sites with 3 mismatches (computational data is available at http://crispr.technology). PCR primers flanking the on-target and predicted off-target sites will be used with a high-fidelity polymerase (NEB, Phusion) to amplify the genomic sequence that will then be tested by the T7EI assay. This will allow us to monitor the targeting accuracy for our optimization experiments both in vitro and in vivo.

2. In vitro evaluation in human cell lines. We have developed several SpCas9 targeting plasmids that contain a unique restriction site for simple target gRNA insertion, and flanking NotI sites to allow for easy subcloning into the ITR containing vector required for AAV production. In addition, we will generate constructs containing the two recently reported high-fidelity Cas9 variants SpCas9-HF and

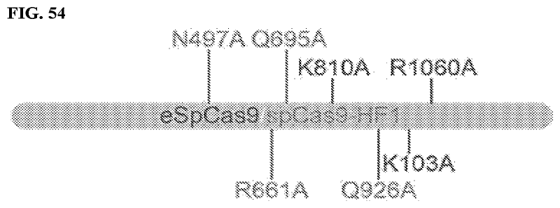

eSpCas926,27 (FIG. 54). We are not aware of any comparisons between these variants and minimizing off-target mutagenesis is paramount to developing safe therapeutics. To ensure that we have efficient targeting at each site, we will first assay the SpCas9 target site for cleavage in HEK293 cells. Using PCR primers flanking the target site we use a high-fidelity polymerase (NEB, Phusion) to amplify the genomic sequence that will then be tested for cleavage activity by the T7EI assay (detailed protocols available at http://crispr.technology).

3. High-throughput sequencing for on-target/off-target mutagenesis. We aim to perform site-specific deep sequencing analysis of on-target and off-target sites. Genomic sequences flanking the CRISPR target site and predicted off-target sites will be amplified using high-fidelity polymerase (NEB, Phusion) for 15 cycles, and then purified (Zymo, DNA Clean & Concentrator-5). Purified PCR products will be amplified for 5 cycles to attach Illumina P5 adapters and sample-specific barcodes, purified again, and then quantitated by SYBR green fluorescence, analyzed on a Bioanalyzer, and finally pooled in an equimolar ratio prior to sequencing with a MiSeq Personal Sequencer. To analyze the sequencing data, 300 bp paired-end MiSeq reads will be de-multiplexed using Illumina MiSeq Reporter software, followed by

adapter and quality trimming of raw reads. Alignments will be performed on all reads to the wild-type sequence and NHEJ frequency will be calculated by: 100*(number of indel reads/number of indel reads+number of WT reads).

4. AAV Virus Production. High titer GMP-like preclinical AAV5 vector will be generated by our collaborator (Dr. William Hauswirth, University of Florida) in their independent vector production facility using the helper-free, plasmid transfection method developed by their lab. Vectors are purified by iodixanol gradient centrifugation followed by Q-column FPLC chromatography, and to establish the

GLP-like purity of the AAV vector stocks, each vector will be subjected to a standardized battery of physical and biological assays including assessment of purity, bioburden, sterility, DNA containing particle titer, infectious titer, particle-to-infectivity ratio and potential contamination by replication competent AAV, each a critical element for the clinical trial IND CMC section.
Development of an LCA10 Therapeutic Mediated by the Co-Delivery of the S. aureus Cas9 (SaCas9) Nickase and Four Guide RNAs Through a Single AAV Vector.

Background and Rationale. Another promising approach that recently emerged to deliver CRISPR by AAV is the use of smaller orthogonal Cas9 proteins.

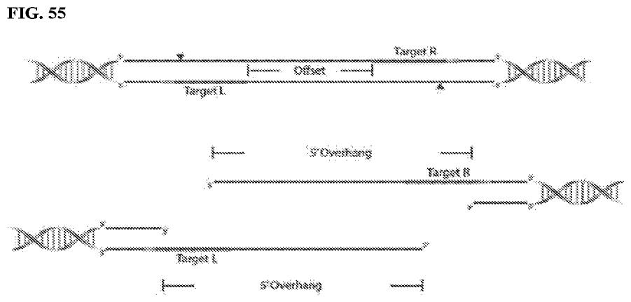

Highlighted by the S. aureus Cas9 (SaCas9), which is encoded by a ˜3.2 kb transcript (Ran, F. A. et al. (2015) Nature 520, 186-191). However, one limitation in using the SaCas9 is due to its PAM requirement (NNGRRT). The number of unique genomic targeting sites is ˜4-fold less than SpCas9 due to the longer the PAM sequence, and there are no SaCas9 sites that fall on the LCA10 A>G mutation. The specific mutation cannot be targeted by SpCas9 (as described above), thus our approach is to employ a deletion strategy to remove a small region surrounding the cryptic exon. The compact size of the SaCas9 gene allows it to be packaged into a single AAV vector along with one, two, or potentially three, gRNA cassettes using standard promoter and terminator elements32. In terms of safety concerns for CRISPR-based therapeutics, the most significant is undoubtedly off-target mutagenesis; this can occur if Cas9 cleaves DNA at an unintended location. Fortunately, this risk can be reduced by several orders of magnitude by employing a point mutation of Cas9, known as a nickase, which only cleaves one DNA strand. By separately engaging two gRNAs to generate two closely opposed nicks on opposite strands, the Cas9 nickase approach can efficiently generate a double-strand break (FIG. 55). Off-target effects can only occur if the two gRNAs recognize off-targets that occur in close proximity and on opposite strands elsewhere in the genome, an occurrence that is statistically very improbable. For this reason, using the nickase to generate a DNA break is the safest approach. With respect to generating a deletion, this approach requires four gRNAs (a pair on each side to delete the targeted mutation)33. However, using the H1 bidirectional system, which provides additional room, we can easily accommodate four gRNAs.

1. Computational selection of gRNAs and construct generation. Similar to our bioinformatics described above, we have identified every SaCas9 targeting site in the genome, and databased the information (data available at http://crispr.technology). In order to target a deletion using the SaCas9 nickase system, we identified four candidate gRNA sites from our computational analysis with favorable properties for generating a deletion using the nickase (Friedland, A. E. et al. (2015) Genome biology 16, 257; Mali, P. et al. (2013) Nat Biotechnol, doi:10.1038/nbt.2675; Ran, F. A. et al. (2013) Cell, doi:10.1016/j.cell.2013.08.021) (FIG. 4).

2. In vitro evaluation in human cell lines. We have constructed SaCas9 targeting plasmids that contain flanking NotI sites to allow for easy subcloning into the ITR containing vector required for AAV production. Like the SpCas9 targeting plasmids, these contain unique restriction sites for rapid cloning of specific gRNA sequences. To ensure that we have efficient targeting at each site, we will first assay each site for dsDNA cutting in HEK293 cells. Using PCR primers flanking the target site we use a high-fidelity polymerase (NEB, Phusion) to amplify the genomic sequence that will then be tested for cleavage activity by the T7EI assay; we routinely identify targeting efficiencies between 30-75%. After these sites have been verified for their ability to induce cleavage, they will be cloned into the SaCas9 nickase targeting plasmid we constructed that contains four H1 promoter cassettes for the expression of four gRNAs. Subsequently, deletion analysis by PCR will be performed in HEK293 cells to assess the efficiency of our targeting constructs. Off-target loci predicted from our bioinformatics will be assessed for spurious mutagenesis.

3. High-throughput sequencing for on-target/off-target mutagenesis. As described above, we will also perform site-specific deep sequencing analysis for our SaCas9 targeting experiments. Given that we are using the nickase approach, we do not expect to detect off-target mutagenesis, however, we have determined alternative SaCas9 targeting sites that are potential alternatives for generating an intronic deletion of the cryptic exon.

4. AAV Virus Production. This will be performed as described above.

### Generation of LCA10 Stem Cell Lines

4 Background and Rationale. While mice can serve as excellent models for retinal degenerations, the rd16 mouse, which is caused by a mutation in CEP290, is not an apt model for testing the CRISPR/AAV therapeutics. Unlike the human point mutation, the mouse degeneration phenotype is caused by a large (897 bp) homozygous in-frame deletion36. Thus, in order to better reflect the human disease, we are currently using two approaches to generate the LCA10 cells: 1) engineering the IVS26 c.2991+1655 A>G mutation into lines from the H7 human embryonic stem cells, and 2) isogenic iPS cell lines derived from the LCA10 patient fibroblasts.

### Experimental Design and Methods.

1. Gene edited hESC lines. We have had success using CRISPR-Cas9 to engineer point mutations using ˜150 base oligo donors (˜75 bases of flanking homology). Plasmids encoding Cas9, the gRNA sequence describe above, and a donor oligo will be introduced by electroporation (Ranganathan, V. et al. (2014) Nature communications 5, 4516). Transfection efficiency will be monitored by fluorescence 24-hrs post-electroporation, and bulk gene targeting will be assessed by T7EI assay. Finally, recombinant clones will be screened for the desired insertion by PCR, and then verified by Sanger sequencing. Multiple homozygous and heterozygous lines carrying the A>G mutation will be isolated and assessed for off-target mutagenesis using the bioinformatics described above.

2. Patient-derived iPSC lines. In collaboration with the Johns Hopkins Wilmer Retinal Degeneration Clinic, we are currently searching for patient fibroblasts harboring the CEP290 mutation. Upon identification, skin biopsies will be collected after informed consent under an existing IRB approved protocol with the JHMI-SOM Institutional Review Board. LCA10 patient-derived iPSCs will be made using established protocols (Galluzzi, L. et al. (2012) Cell death and differentiation 19, 107-120; Yu, J. et al. (2007) Science 318, 1917-1920; Takahashi, K. et al. (2006) Cell 126, 663-676; Ludwig, T. E. et al. (2006) Nat Biotechnol 24, 185-187). Using the techniques described above, we will generate mutation corrected isogenic cell lines in parallel, and the predicted off-target loci will be Sanger sequenced to verify the absence of extraneous mutations.

Bioethics. We will be using the human Embryonic Stem Cell line H7 (WiCell); use of these cell lines for the proposed experiments is approved by JHU ISCRO committee (application ISCRO00000023). In addition, we will be using hiPSC generated in our lab under informed consent under the auspices of the JHU Institutional review board (IRB #NA_00047271). Although not considered an ES cell line, we still follow all policies and regulations dictated by the JHU Embryonic Stem Cell Research Oversight committee.

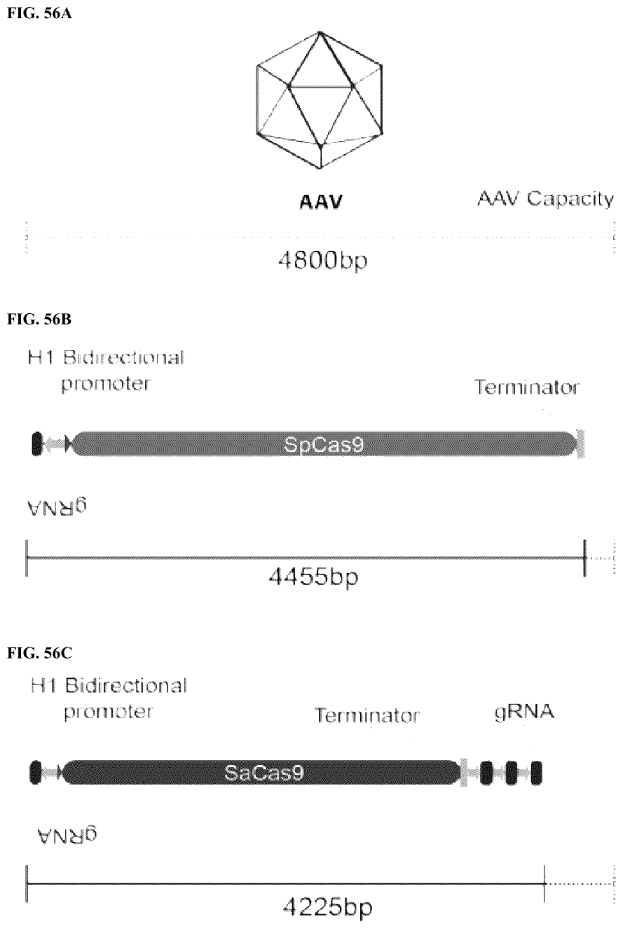

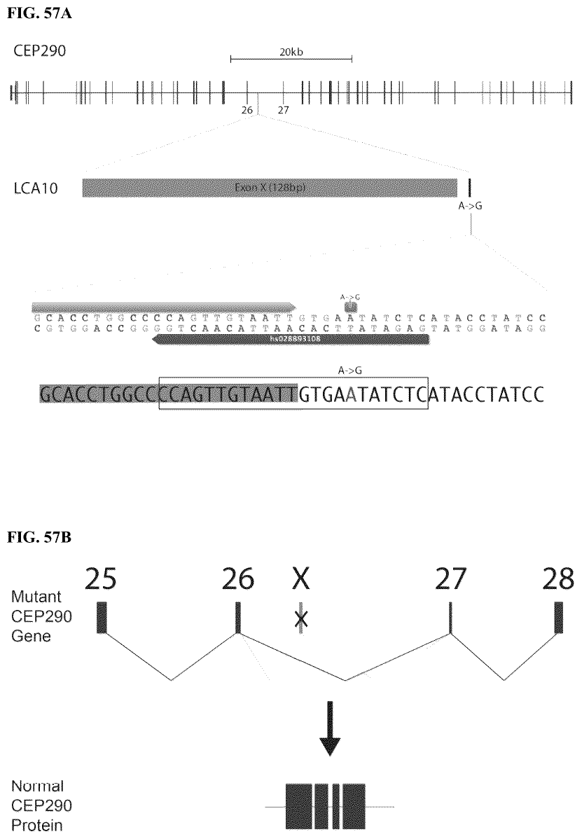

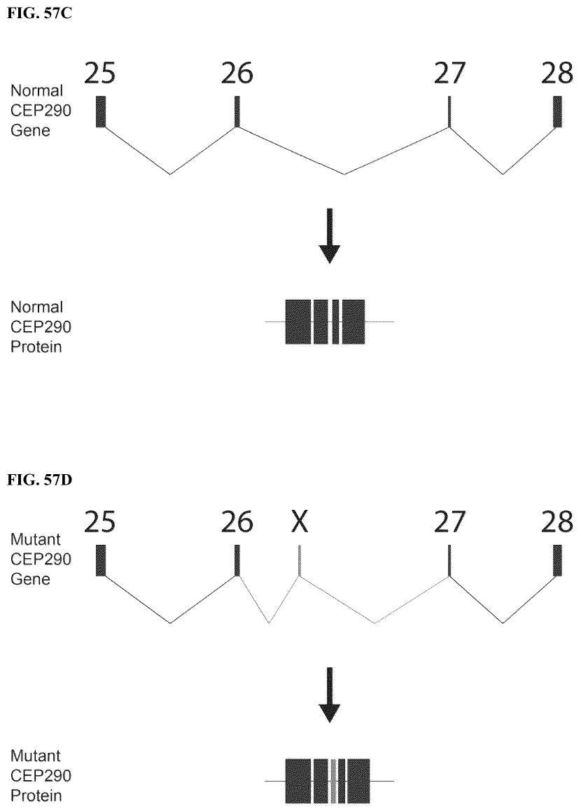

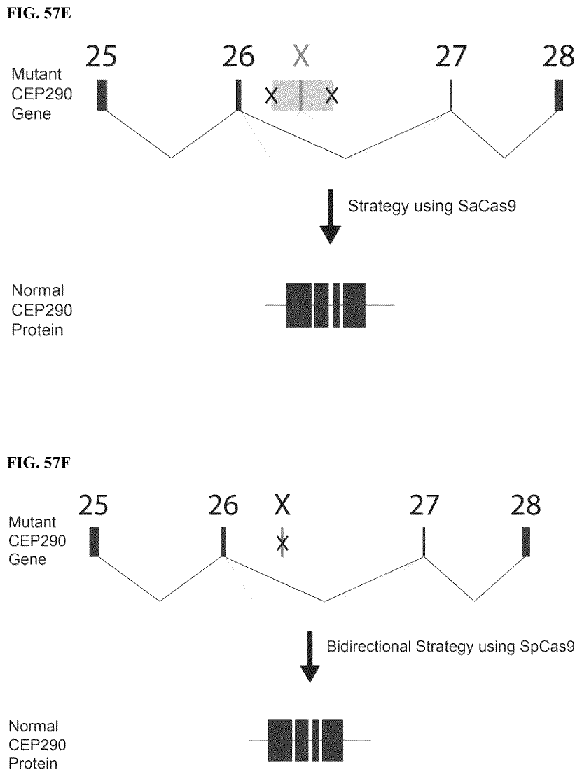

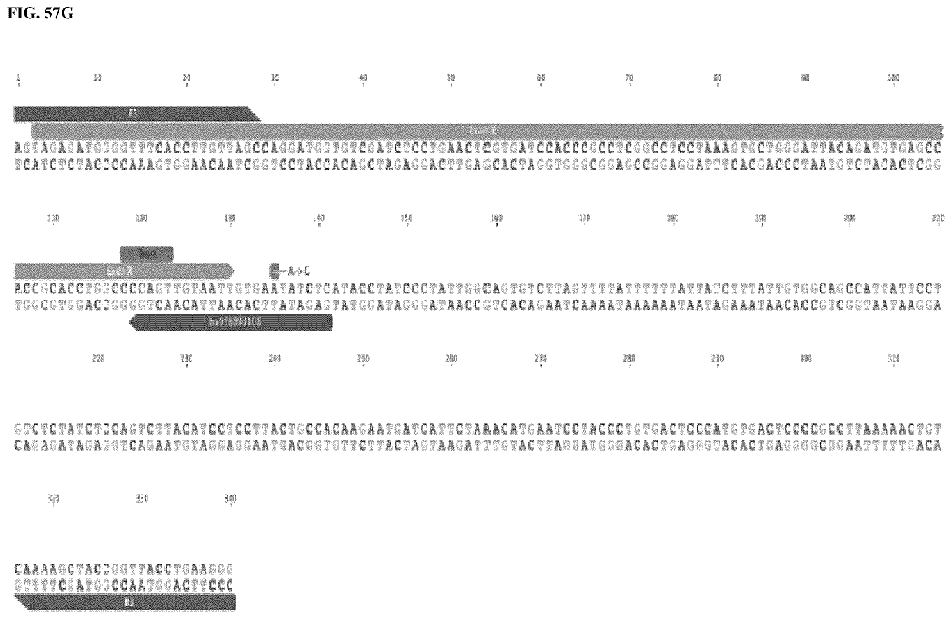

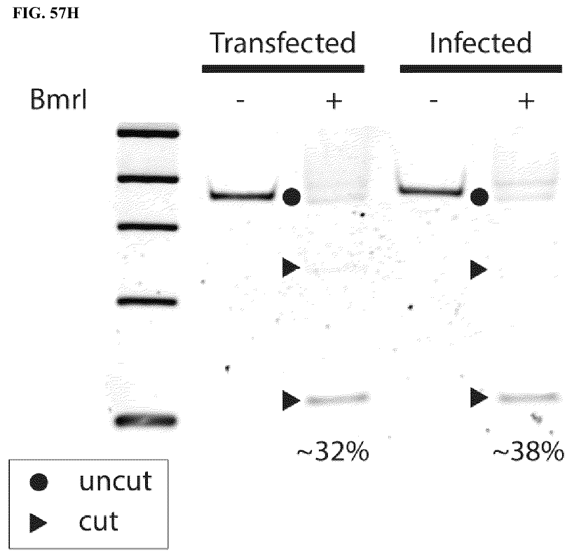

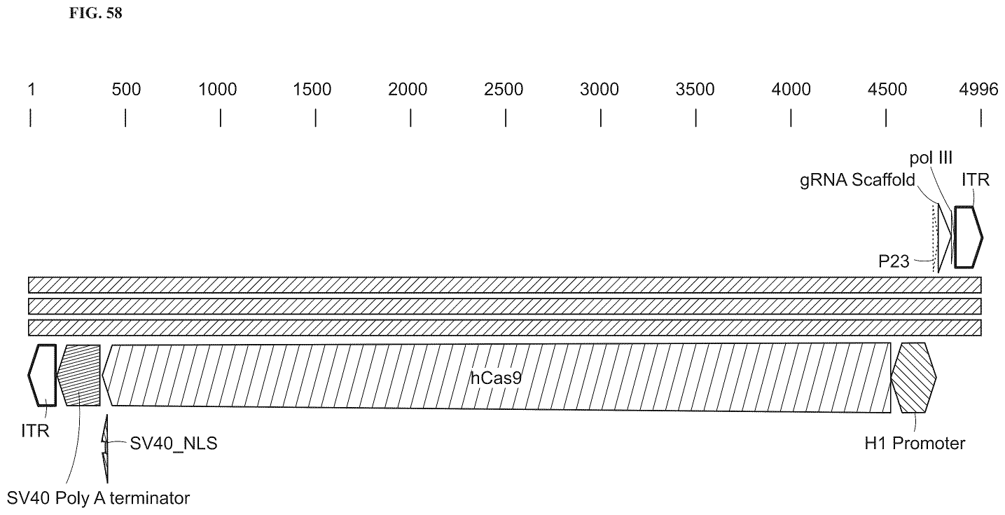

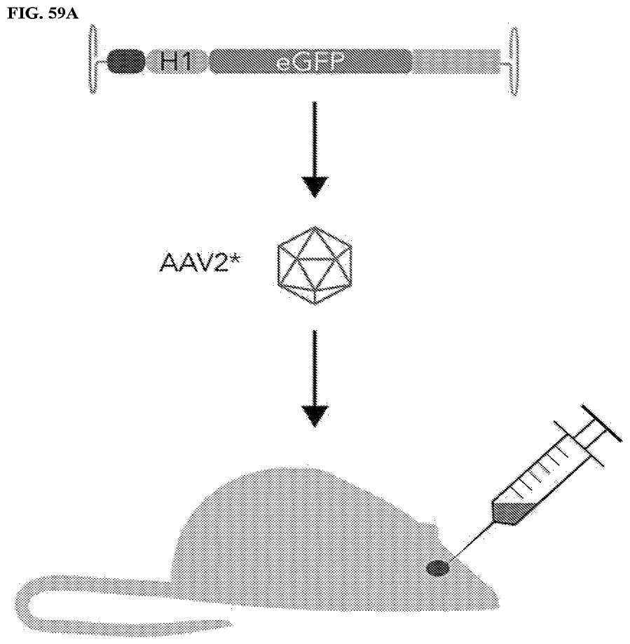

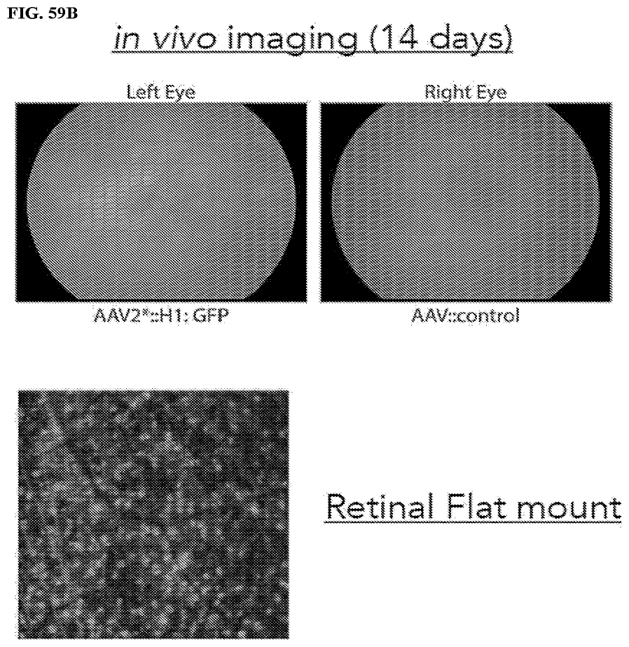

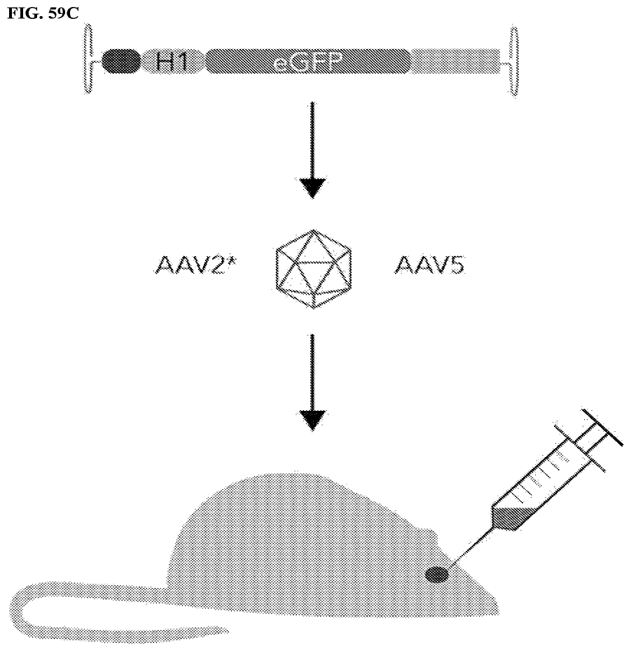

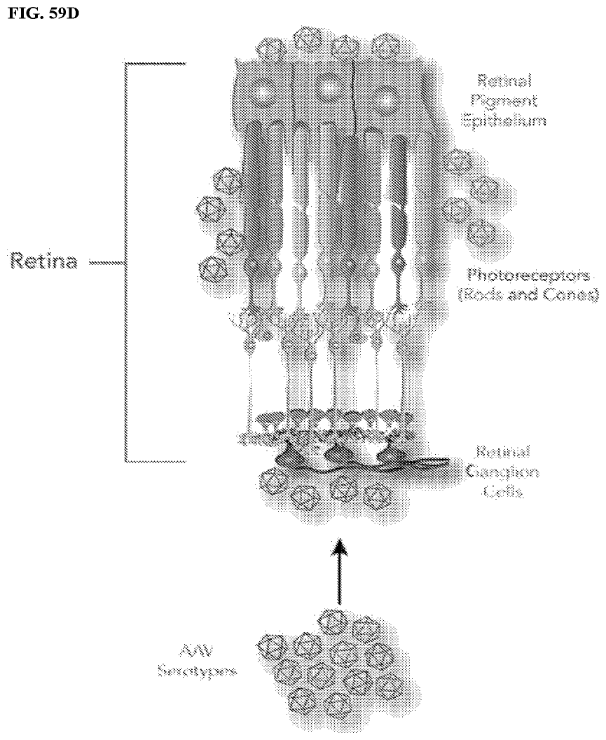

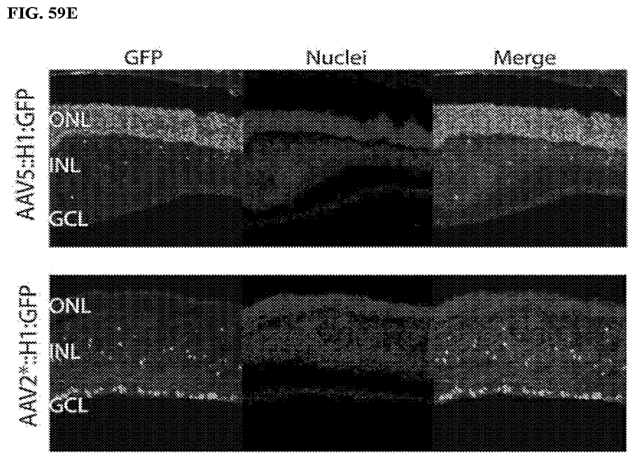

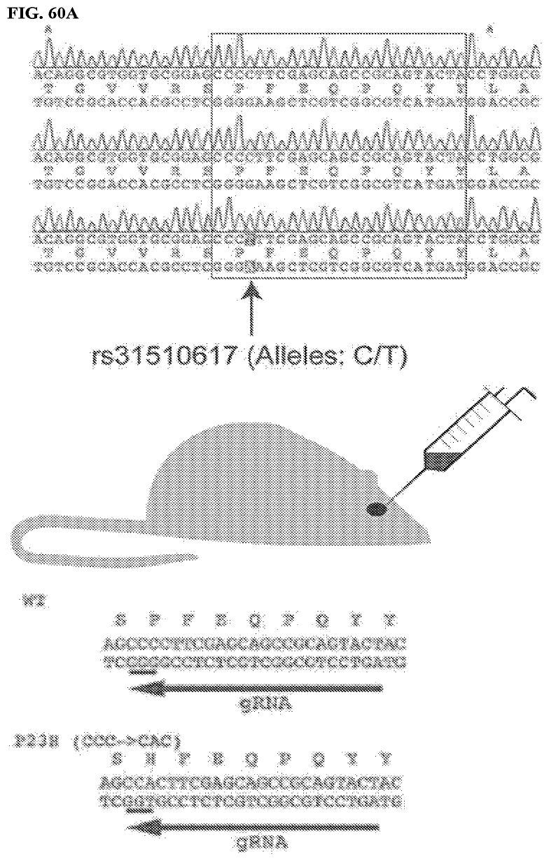

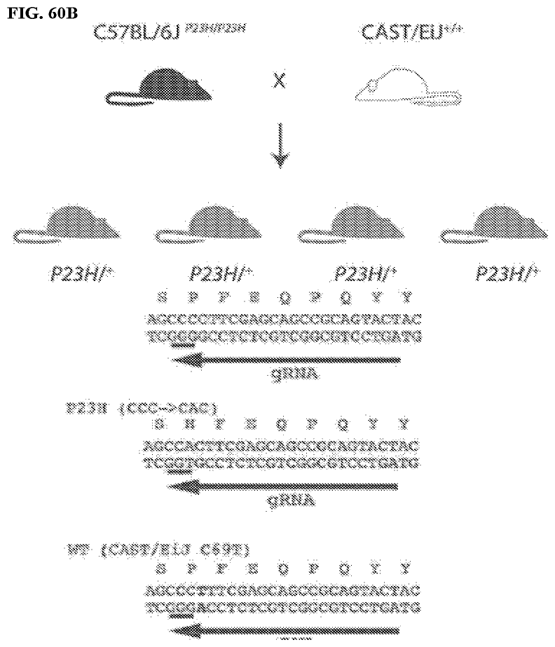

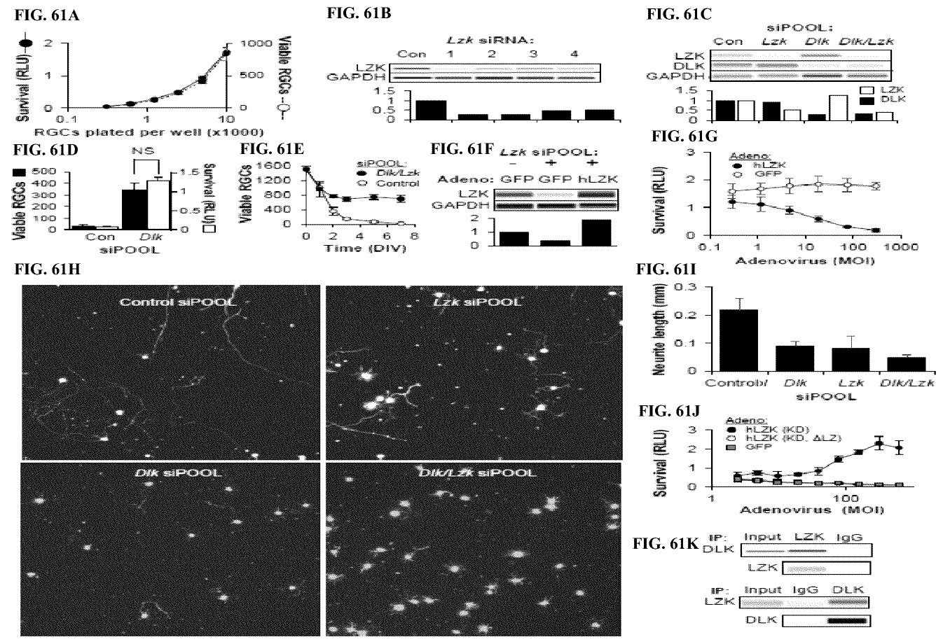

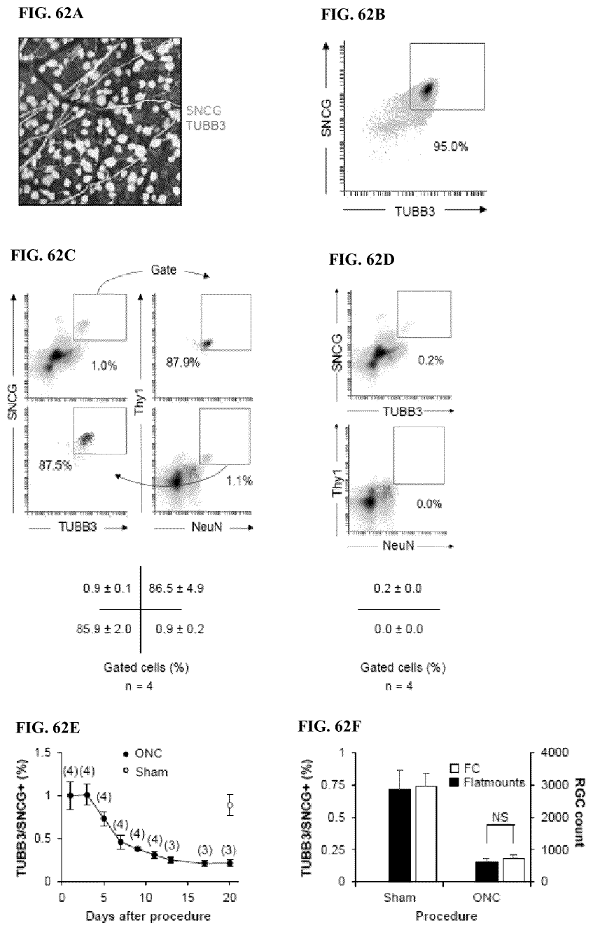

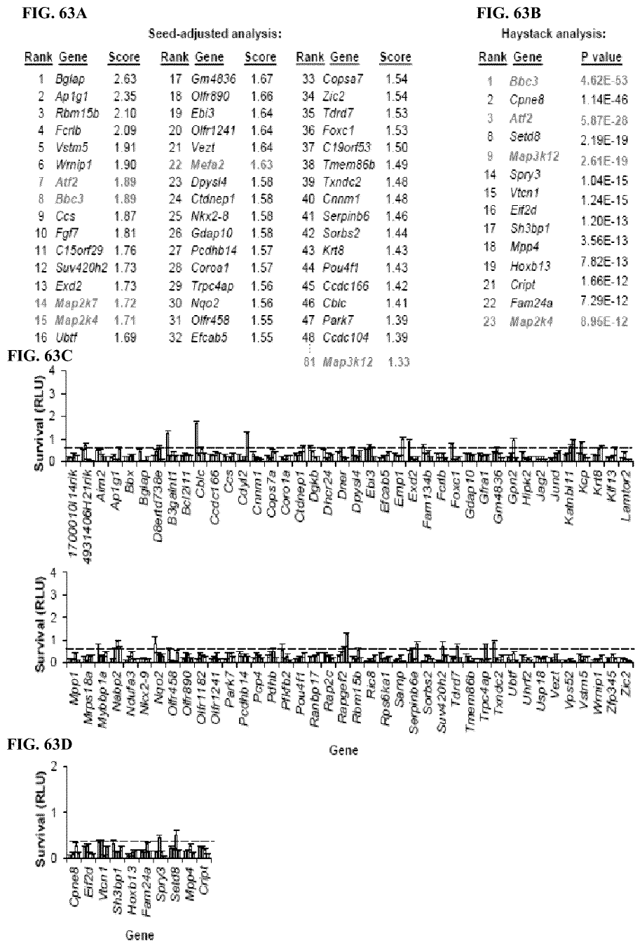

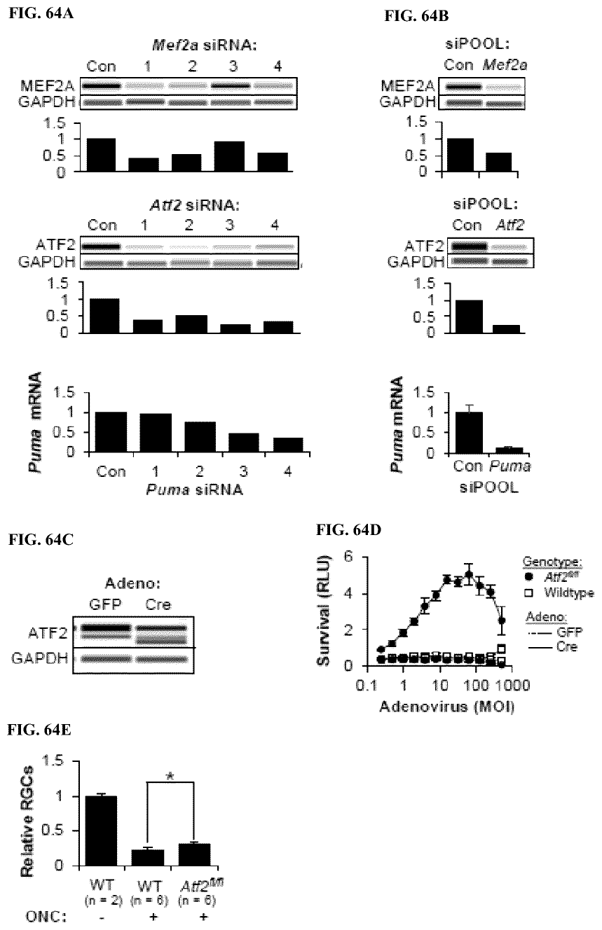

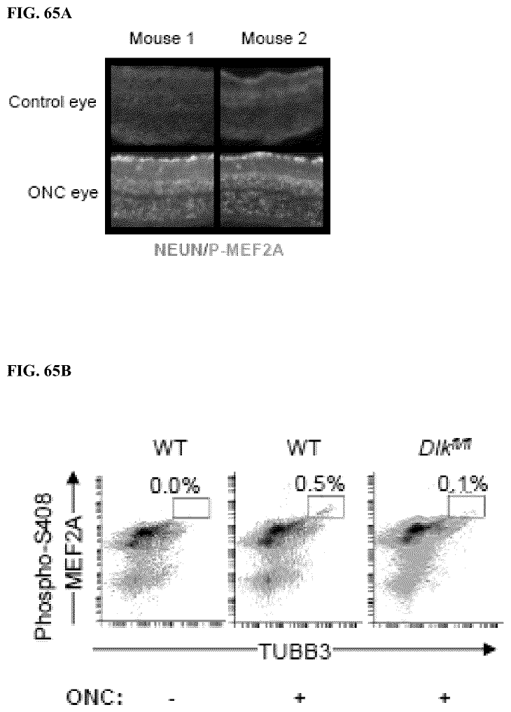

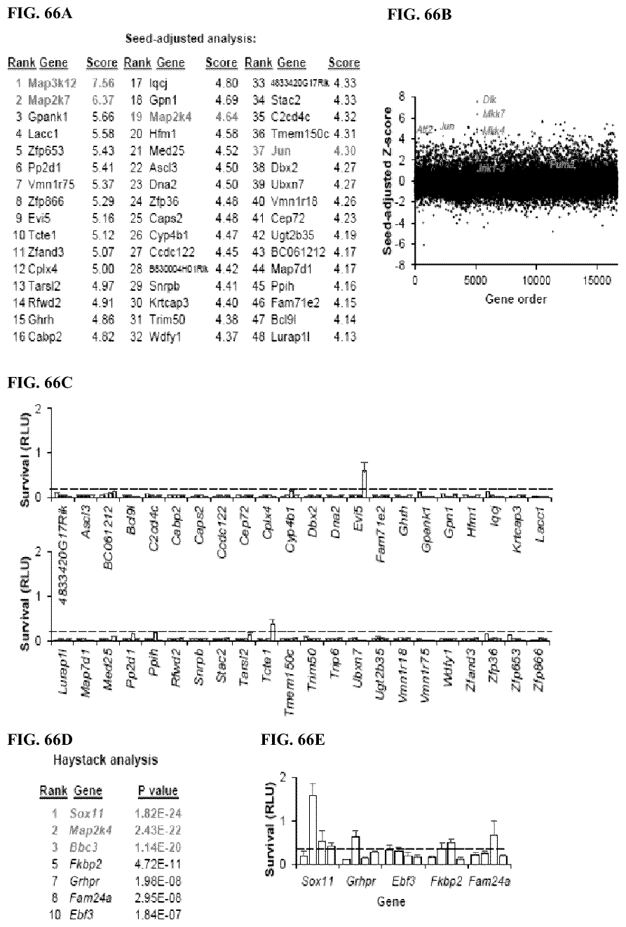

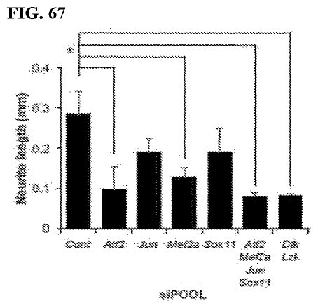

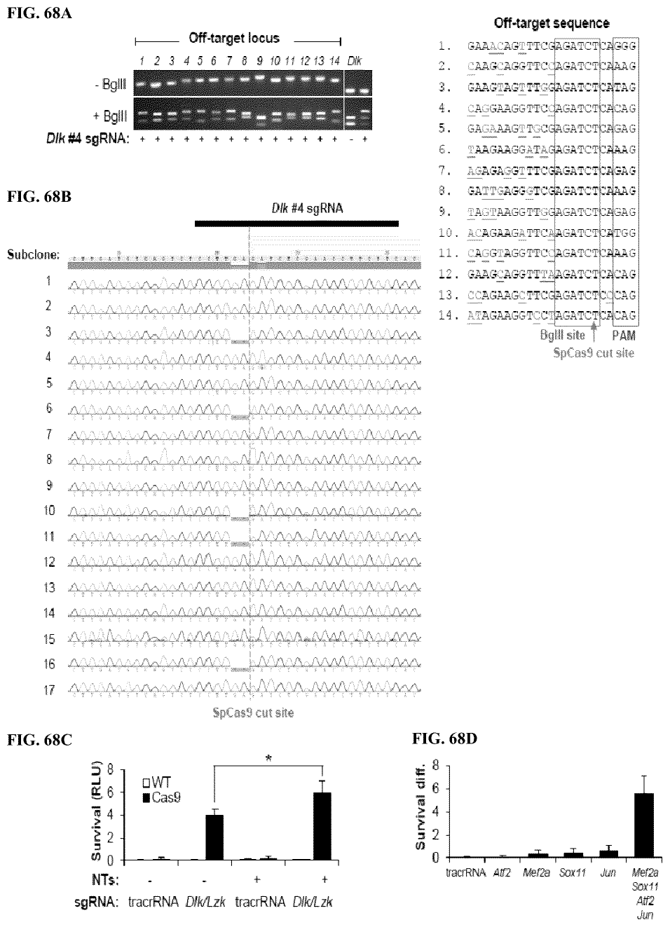

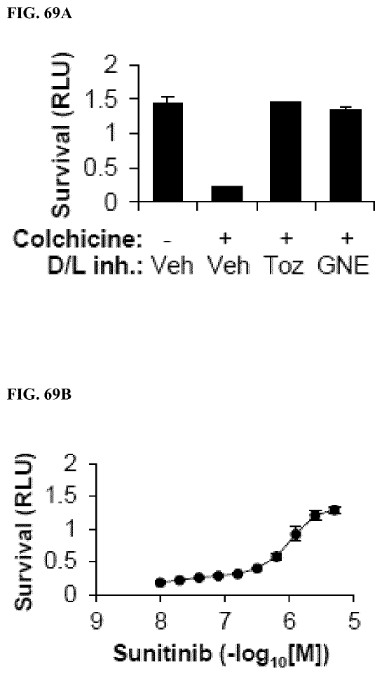

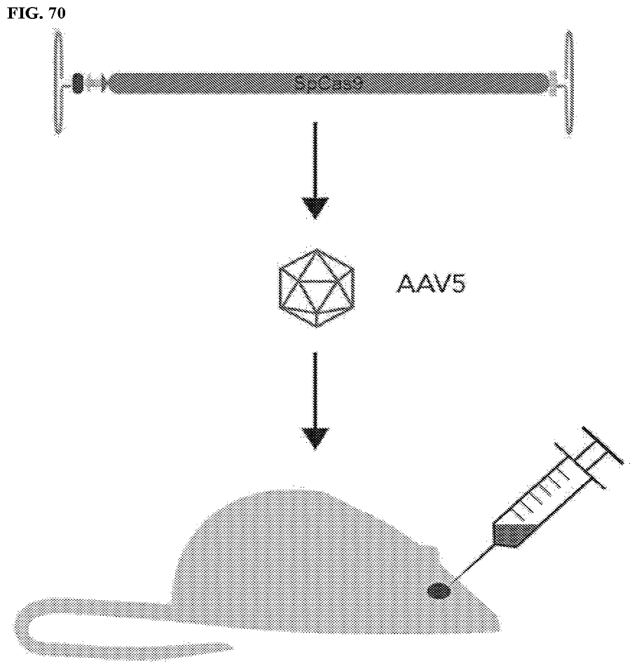

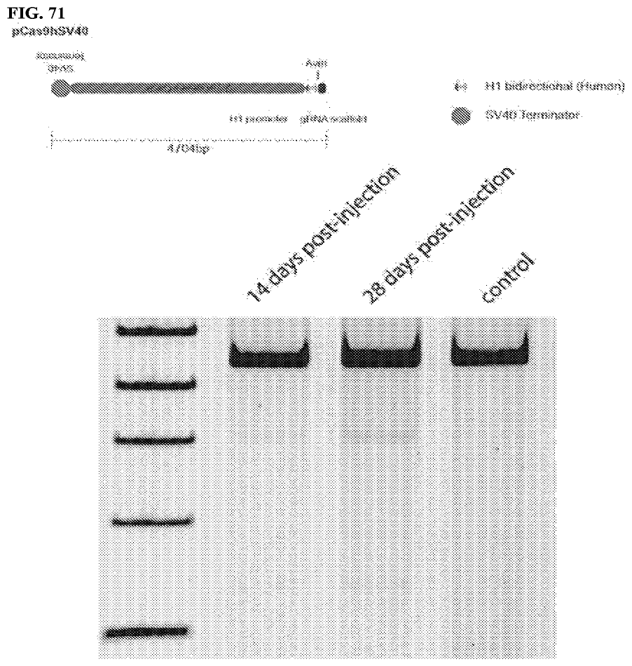

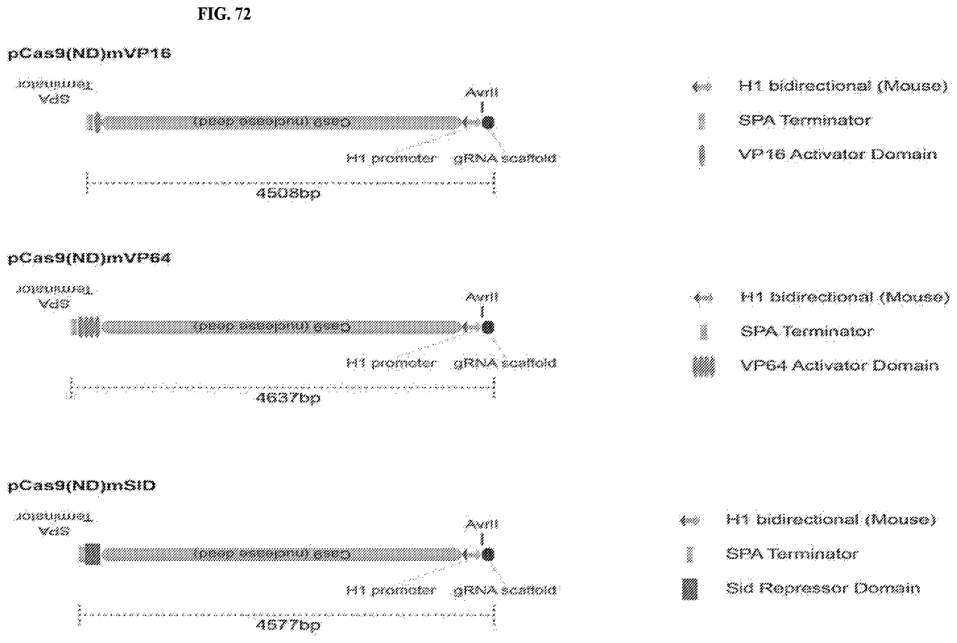

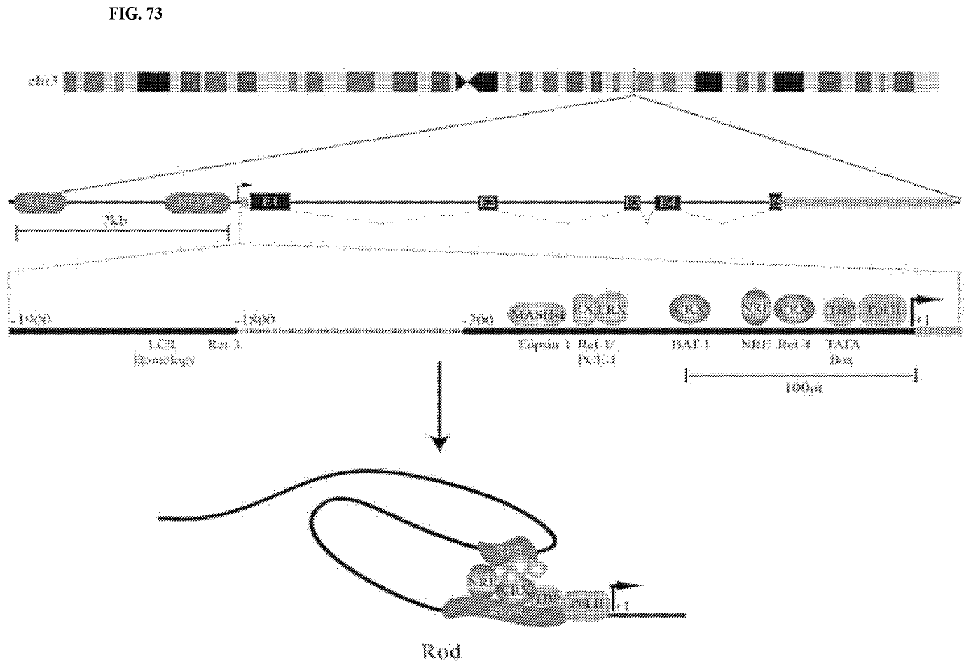

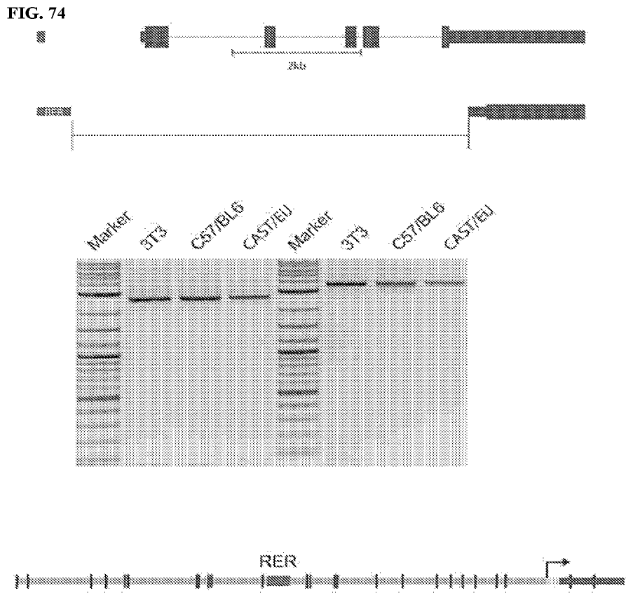

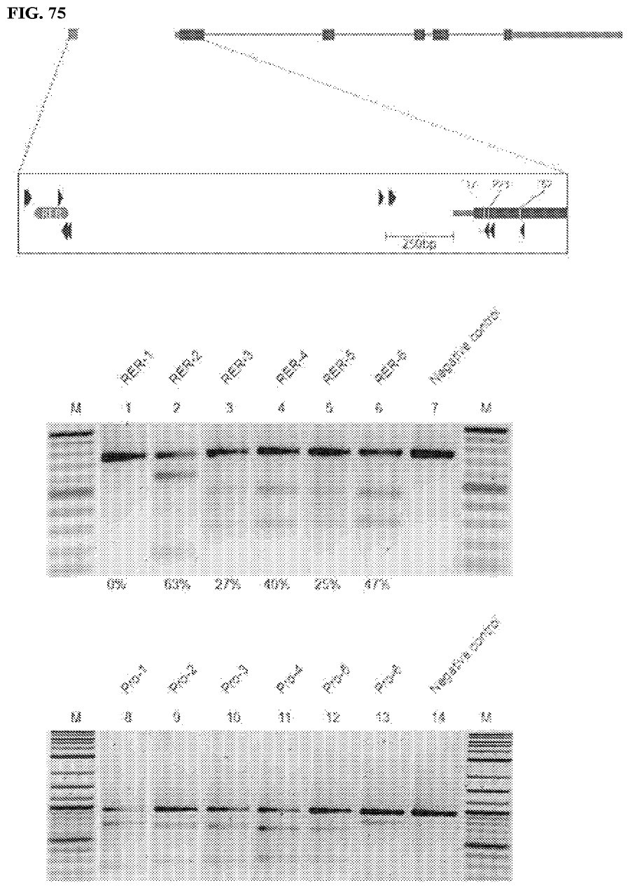

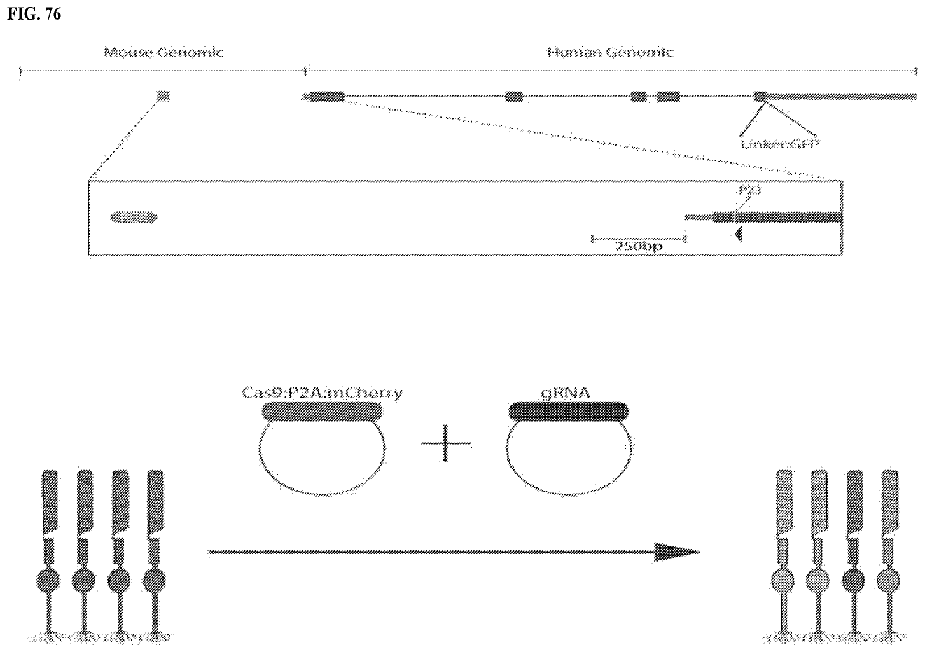

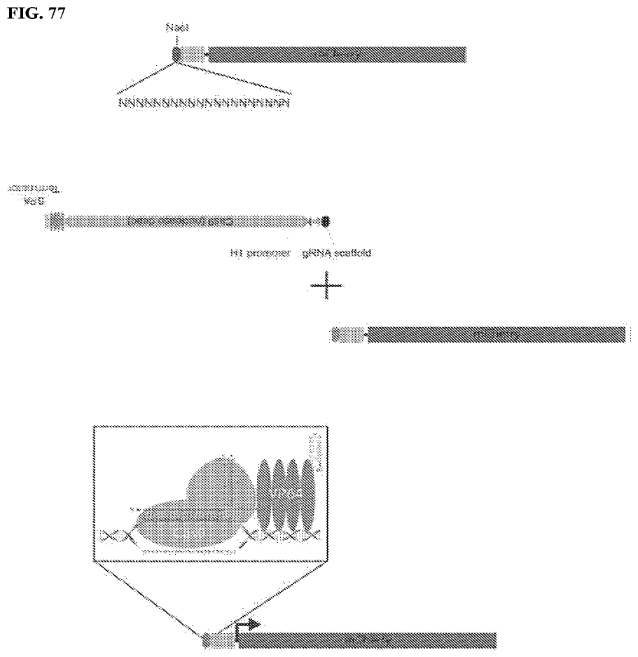

3. in vitro editing of gene edited hESC lines and/or editing of patient derived iPSC lines. In order to thoroughly assess the various lead viral vectors developed in above, cells from either patient derived iPSC lines or gene edited hESC lines will be tested; the constructs that target the LCA10 mutation directly with WT SpCas9, eSpCas9, or the SpCas9-HF, and the construct that uses the SaCas9 nickase approach with four gRNA (FIG. 56). First, on-target cutting efficiency will be determined by the T7EI assay. For high-resolution analysis, MiSeq will be used to quantitate the on-target and off-target mutagenesis. Since SpCas9 targeting will rely on error-prone NHEJ to eliminate critical nucleotides for splicing, this analysis will provide a good indication of the therapeutic potential. In addition to cutting efficiency and off-target mutagenesis, qRTPCR will be used to quantitate the levels of CEP290 expression from both mutant and wild-type alleles. This will provide a first step in validating our constructs. With our existing capabilities in the lab, quantitative ciliogenesis assays (Kim, J. et al. (2010) Nature 464, 1048-1051) can be performed using our high content machine to assess the potential of our therapeutic constructs. Additionally, we have developed protocols for the differentiation of stem cells into photoreceptors allowing us to assess the phenotypic rescue in the relevant cell type. Given the lack of good animal models for assessing genome editing for LCA10, we believe these assays will more closely approximate the true therapeutic potential of our viral constructs.

### Example 6

AAV5 was delivered by sub-retinal injection to P0.5 mice. Following either 14 days of 28 days, the retina was harvested and a T7 Endo I assay was performed. (Note: AAV5 targets rod photoreceptors, and the assay was performed on total retina).

For T7 EndoI analysis, genomic DNA was extracted by resuspending cells in QuickExtract solution (Epicentre, Madison, WI), incubating at 65° C. for 20 min, and then at 98° C. for 20 min. The extract solution was used directly or cleaned using DNA Clean and Concentrator (Zymo Research, Irvine, CA), and quantitated by NanoDrop (Thermo 30 Fisher Scientific). The genomic region surrounding the CRISPR target site was amplified from ˜100 ng of genomic DNA using Phusion DNA polymerase (New England Biolabs). Multiple independent PCR reactions were pooled and purified using DNA Clean and Concentrator (Zymo Research, Irvine, CA). A 25 μl volume containing 150 ng of the PCR product in 10 mM Tris-HCl, 50 mM NaCl, 10 mM MgCl₂ and 100 μg/ml BSA was denatured and slowly reannealed to allow for the formation of heteroduplexes: 95° C. for 10 min, 95° C. to 85° C. ramped at −1.0° C./sec, 85° C. for 1 sec, 85° C. to 75° C. ramped at −1.0° C./sec, 75° C. for 1 sec, 75° C. to 65° C. ramped 5 at −1.0° C./sec, 65° C. for 1 sec, 65° C. to 55° C. ramped at −1.0° C./sec, 55° C. for 1 sec, 55° C. to 45° C. ramped at −1.0° C./sec, 45° C. for 1 sec, 45° C. to 35° C. ramped at −1.0° C./sec, 35° C. for 1 sec, 35° C. to 25° C. ramped at −1.0° C./sec, and then held at 4° C. 1 μl of T7 EndoI (New England Biolabs) were added to each reaction, incubated at 37° C. for 30 min, and then immediately placed on ice. For gel analysis, 3 μl of the reaction was mixed with 3 μl 2× Loading dye (New England Biolabs), loaded onto a 6% TBE-PAGE gel, and stained with SYBR Gold (1:10,000) for ˜15 minutes prior to visualization. Gels were visualized on a Gel Logic 200 Imaging System (Kodak, 15 Rochester, NY), and quantitated using ImageJ v. 1.46. NHEJ frequencies were calculated using the binomial-derived equation:

% gene modification=100*(1−(SQRT(1−((a+b)/(a+b+c)))))

where the values of “a” and “b” are equal to the integrated area of the cleaved fragments after background subtraction and “c” is equal to the integrated area of the un-cleaved PCR product after background subtraction.
1. Levkovitch-Verbin H et al. iovsorg 44, 3388-3393 (2003).
2. Howell G R et al. J Cell Biol 179, 1523-1537 (2007).
3. Adalbert R et al. Science (2012), doi:10.1126/science.1223899.
4. Yang J et al. Cell 160, 161-176 (2015).
5. Welsbie D S et al. Proc Nat Acad Sci USA 110, 4045-4050 (2013).
6. Watkins T A et al. Proc Nat Acad Sci USA 110, 4039-4044 (2013).
7. Barres B A et al. Neuron 1, 791-803 (1988).
8. Miller B R et al. Nat Neurosci 12, 387-389 (2009).
9. Petrs-Silva H et al. Mol Ther 19, 293-301 (2011).
10. Petrs-Silva H et al. Mol Ther 17, 463-471 (2009).
11. Sun H et al. Mol Vis 17, 864-875 (2011).
12. Ribas V T et al. Neuroscience 180, 64-74 (2011).
13. Davis M I et al. Nat Biotechnol 29, 1046-1051 (2011).
14. Karaman M W et al. Nat Biotechnol 26, 127-132 (2008).
15. Bounoutas A et al. Proc Nat Acad Sci USA 108, 3982-3987 (2011).
16. Takihara Y et al. Invest Ophthalmol Vis Sci 52, 3039-3045 (2011).
17. Jackson A L et al. RNA 12, 1179-1187 (2006).
18. Jackson A L et al. Nat Biotechnol 21, 635-637 (2003).
19. Marine S Journal of Biomolecular Screening 17, 370 (2011).
20. Hannus M et al. Nucleic Acids Res 42, 8049-8061 (2014).
21. Chen S et al. Cell 159, 440 (2014).
22. Buehler E et al. Sci Rep 2, 428 (2012).
23. Yoshida K et al. Invest Ophthalmol Vis Sci 43, 1631-1635 (2002).
24. Bhoumik A et al. Proc Nat Acad Sci USA 101, 4222-4227 (2004).
25. Jiang Y et al. J Biol Chem 288, 18429-18438 (2013).
26. Lin L et al. Dev Dyn 240, 52-64 (2010).
27. Okamoto S et al. Proc Nat Acad Sci USA 97, 7561-7566 (2000).
28. Ornatsky O I et al. Journal of Biological Chemistry 272, 33271-33278 (1997).
29. Shalizi A et al. Science 311, 1012-1017 (2006).
30. Akhtar M W et al. PLoS ONE 7, e34863 (2012).
31. Xiong X et al. J Cell Biol 191, 211-223 (2010).
32. Fernandes K A et al. Neurobiol Dis 69, 108-116 (2014).
33. de Lima S et al. Proc Nat Acad Sci USA 109, 9149-9154 (2012).
34. Smith P D et al. Neuron 64, 617-623 (2009).
35. Sun F et al. Nature 480, 372-375 (2011).
36. Babetto E et al. Cell Rep 3, 1422-1429 (2013).
37. Huntwork-Rodriguez S et al. J Cell Biol 202, 747-763 (2013).
38. Culican S M et al. Mol Cell Neurosci 41, 304-312 (2009).
39. Vo B Q et al. Vis Neurosci 28, 175-181 (2011).
40. Yan D et al. Neuron 76, 534-548 (2012).
41. Kampmann M et al. Proc Nat Acad Sci USA 112, E3384-91 (2015).
42. González F et al. Cell Stem Cell 15, 215-226 (2014).
43. Wong N et al. Genome Biol 16, 218 (2015).
44. Doench J G et al. Nat Biotechnol 32, 1262-1267 (2014).
45. Yang L et al. Nat Commun 5, 5507 (2014).
46. Varshney G K et al. Genome Res 25, 1030-1042 (2015).
47. Wang T H et al. J Biol Chem 273, 4928-4936 (1998).
48. Hu J et al. Biochimica et Biophysica Acta (BBA)—Proteins and Proteomics 1844, 224-231 (2014).
49. Fiedler D et al. Cell 136, 952-963 (2009).
50. Wu C C et al. Cell Cycle 14, 1207 (2015).
51. Swiech L et al. Nat Biotechnol 33, 102-106 (2015).
52. Ran F A et al. Nature 520, 186-191 (2015).
53. Ranganathan V et al. Nat Commun 5, 4516 (2014).
54. Baer M et al. Nucleic Acids Res 18, 97-103 (1990).
55. Myslinski E Nucleic Acids Res 29, 2502 (2001).
56. Koilkonda R et al. Mol Vis 15, 2796 (2009).
57. Bemelmans A P et al. PLoS ONE 8, e61618 (2013).
58. Byrne L C et al. Mol Ther 23, 290 (2014).
59. Hirai S I et al. J Neurosci 31, 6468-6480 (2011).
60. Bei F et al. Cell 164, 219-232 (2016).
61. Ramakrishna S et al. Nat Commun 5, 3378 (2014).
62. Tsai S Q et al. Nat Biotechnol 33, 187-197 (2015).
63. Parsons B D et al. PLoS ONE 4, e8471 (2009).
64. Slaymaker I M et al. 351, 84-88 (2015).
65. Kleinstiver B P et al. Nature (2016).
66. Fu Y et al. Nat Biotechnol 32, 279-284 (2014).
67. Ran F A et al. Cell 154, 1380-1389 (2013).
68. Potthoff M J Development 134, 4131-4140 (2007).
69. Okamoto S I et al. Proc Nat Acad Sci USA 99, 3974-3979 (2002).
70. Gong X et al. Neuron 38, 33-46 (2003).
71. Flavell S W et al. Science 311, 1008-1012 (2006).
72. Thomas S N et al. Anal Chem 87, 10830-10838 (2015).
73. Aiyetan P et al. BMC Bioinformatics 16, 411 (2015).

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

1. A method for treating a retinal degeneration in a subject in need thereof, the method comprising:
(a) providing a non-naturally occurring nuclease system comprising one or more vectors comprising:
i) a promoter operably linked to at least one nucleotide sequence encoding a nuclease system guide RNA (gRNA), wherein the gRNA hybridizes with a target sequence of a DNA molecule in a cell of the subject, and wherein the DNA molecule encodes one or more gene products expressed in the cell, wherein the one or more gene products comprise Dual Leucine Zipper Kinase (DLK) and Leucine Zipper Kinase (LZK); and
ii) a regulatory element operable in a cell operably linked to a nucleotide sequence encoding a genome-targeted nuclease,
wherein components (i) and (ii) are located on the same or different vectors of the system, wherein the gRNA targets and hybridizes with the target sequence and the nuclease cleaves one or two strands of the DNA molecule to alter expression of the one or more gene products; and
(b) administering to a retinal area of the subject a therapeutically effective amount of the system.

2. The method of claim 1, wherein the system is CRISPR.

3. The method of claim 1, wherein the system is packaged into a single adeno-associated virus (AAV) particle.

4. The method of claim 1, wherein the system inactivates the one or more gene products.

5. (canceled)

6. The method of claim 1, wherein the promoter is a bidirectional promoter.

7. The method of claim 6, wherein the bidirectional promoter is H1.

8. The method of claim 7, wherein the H1 promoter comprises:
a) control elements that provide for transcription in one direction of the at least one nucleotide sequence encoding the gRNA; and
b) control elements that provide for transcription in the opposite direction of the nucleotide sequence encoding the genome-targeted nuclease.

9. The method of claim 1, wherein the genome-targeted nuclease is Cas9 protein.

10. The method of claim 9, wherein the Cas9 protein is codon optimized for expression in the cell.

11. The method of claim 6, wherein the promoter is operably linked to at least one, two, three, four, five, six, seven, eight, nine, or ten gRNA.

12. The method of claim 1, wherein the retinal area is the retina.

13. (canceled)

14. The method of claim 1, wherein the cell is a retinal ganglion cell.

15. The method of claim 1, wherein the retinal degeneration is glaucoma.

16-29. (canceled)

30. The method of claim 15, wherein the retinal degeneration is glaucoma.

31-32. (canceled)

33. The method of claim 1, wherein the target sequence is selected from the group consisting of the nucleotide sequences set forth in any one of SEQ ID NOs: 143-163 or combinations thereof.

34-36. (canceled)

37. A method of altering expression of one or more gene products in a cell, wherein the cell comprises a DNA molecule encoding the one or more gene products, wherein the one or more gene products comprise Dual Leucine Zipper Kinase (DLK) and Leucine Zipper Kinase (LZK), the method comprising introducing into the cell a non-naturally occurring nuclease system comprising one or more vectors comprising:
a) a promoter operably linked to at least one nucleotide sequence encoding a nuclease system guide RNA (gRNA), wherein the gRNA hybridizes with a target sequence of the DNA molecule; and
b) a regulatory element operable in the cell operably linked to a nucleotide sequence encoding a genome-targeted nuclease,
wherein components (a) and (b) are located on the same or different vectors of the system, wherein the gRNA targets and hybridizes with the target sequence and the nuclease cleaves the DNA molecule to alter expression of the one or more gene products.

38-47. (canceled)

48. The method of claim 37, wherein:
(a) the cell is a eukaryotic or non-eukaryotic cell;
(b) the system is CRISPR;
(c) the system is packaged into a single adeno-associated virus (AAV) particle;
(d) the system inactivates the one or more gene products;
(e) the genome-targeted nuclease is Cas9 protein; and/or
(f) the promoter is operably linked to at least one, two, three, four, five, six, seven, eight, nine, or ten gRNA.

49. The method of claim 48, wherein:
(a) the eukaryotic cell is a mammalian or human cell; and/or
(b) the Cas9 protein is codon optimized for expression in the cell.

50-65. (canceled)

66. The method of claim 37, wherein the promoter is a bidirectional promoter.

67. The method of claim 66, wherein the bidirectional promoter is H1.
